(19) United States
(12) Patent Application Publication
(10) Pub. No.: US 2024/0116870 A1
(43) Pub. Date: Apr. 11, 2024

(54) N,N-DIMETHYLTRYPTAMINE AND RELATED PSYCHEDELICS AND USES THEREOF

(71) Applicant: Terran Biosciences Inc., New York, NY (US)

(72) Inventors: Matthew Alexander James DUNCTON, San Bruno, CA (US); Sam CLARK, New York, NY (US)

(21) Appl. No.: 18/484,211

(22) Filed: Oct. 10, 2023

Related U.S. Application Data
(63) Continuation of application No. 18/173,717, filed on Feb. 23, 2023.
Application No. 18/173,717 is a continuation of application No. PCT/US2022/036396, filed on Jul. 7, 2022.
(60) Provisional application No. 63/276,516, filed on Nov. 5, 2021.
(60) Provisional application No. 63/219,312, filed on Jul. 7, 2021.

(51) Int. Cl. C07D 209/32; C07D 209/12; C07D 209/14

(52) U.S. Cl. CPC C07D 209/32; C07D 209/12; C07D 209/14

(57) ABSTRACT

Described herein are compounds that are derivatives of DMT or 5-MeO-DMT and can be metabolically converted to N,N-dimethyltryptamine or analogs thereof upon administration to a subject. In certain embodiments, the compounds described herein are useful for the treatment of conditions associated with a neurological disease.

### CROSS REFERENCE TO RELATED APPLICATIONS

This application is a continuation of U.S. patent application Ser. No. 18/173,717, filed Feb. 23, 2023, which is a continuation of international patent application no. PCT/US2022/036396 filed on Jul. 7, 2022, which claims the benefit of U.S. Provisional Patent Application No. 63/219,312, filed Jul. 7, 2021, and U.S. Provisional Patent Application No. 63/276,516, filed on Nov. 5, 2021, the contents of each is incorporated by reference herein in their entireties.

### BACKGROUND OF THE INVENTION

Nearly 1 in 5 adults in the United States suffer from mental illness, and over 50% of Americans will be diagnosed with a psychiatric disorder at some point in their lifetime. 1 in 25 Americans is afflicted with severe mental illness, such as major depression, schizophrenia, or bipolar disorder.

### SUMMARY OF THE INVENTION

In one aspect, the present disclosure provides a compound of Formula (I), or a pharmaceutically acceptable salt thereof:

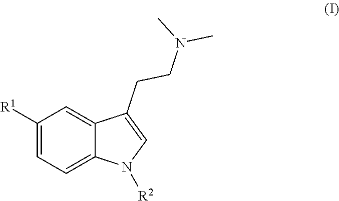

wherein:
R¹ is methoxy or hydrogen;
R² is —C(O)OR³, —C(O)R⁴, —CH(R⁵)OR⁶, —C(O)OCH(R⁵)OC(O)R⁶, —C(O)OCH(R⁵)OC(O)OR⁶, —CH(R⁵)C(O)R⁶, —CH(R⁵)OC(O)R⁶, —CH(R⁵)OC(O)OR⁶, —S(O)₂OR⁷, —P(O)OR⁹[N(R⁹)R¹⁰], —P(O)[N(R⁹)R¹⁰][N(R¹¹)R¹²], —C(O)N(R⁹)R¹⁰, —P(O)OR¹¹(OR¹²), —CH(R⁵)OP(O)OR⁹[N(R⁹)R¹⁰], or —CH(R⁵)OP(O)OR¹¹(OR¹²); each of R³, R⁴, R⁵, R⁶, R⁷, and R⁸ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently unsubstituted or substituted with one or more R^{A};
each of R⁹ and R¹⁰ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R⁹ and R¹⁰ together with the atom to which they are attached form a heterocyclylalkyl ring that is unsubstituted or substituted with one or more R^{A};
each of R¹¹ and R¹² is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R¹¹ and R¹² together with the atoms to which they are attached form a heterocyclylalkyl ring that is unsubstituted or substituted with one or more R^{A};
each R^{A} is independently alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹]—C(O)N(R¹⁸)R¹⁹, —OC(O)N(R¹⁸)R¹⁹, or —OP(O)OR²⁰(OR²¹), wherein each alkyl, heteroalkyl, cycloalkyl, heterocyclylalkyl, amino acid side chain, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B};
each of R¹⁸ and R¹⁹ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B}; or
R¹⁸ and R¹⁹ together with the atom to which they are attached form a heterocyclylalkyl ring that is unsubstituted or substituted with one or more R^{B};
each of R²⁰ and R²¹ is independently alkyl, alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B}, or R²⁰ and R²¹ together with the atoms to which they are attached form a heterocyclylalkyl ring that is unsubstituted or substituted with one or more R^{B}; and
each R^{B} is independently halogen, amino, cyano, hydroxyl, alkyl, heteroalkyl, cycloalkyl, heterocyclylalkyl, aryl, heteroaryl, arylalkyl, —C(O)CH₃, —C(O)Ph, or heteroarylalkyl, wherein each cycloalkyl, heterocyclylalkyl, aryl, and heteroaryl is independently unsubstituted or substituted with one or more halogen, amino, cyano, hydroxyl, alkyl, acetyl, or benzoyl,
provided that when R¹ is hydrogen, then R³ is not tert-butyl.

In some embodiments, the compound of Formula (I) having the structure of Formula (Ia), or a pharmaceutically acceptable salt thereof:

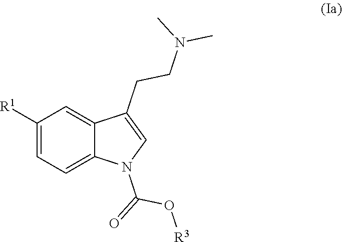

wherein R¹ is methoxy or hydrogen, and R³ is alkyl, cycloalkyl, aryl, heteroaryl, heteroalkyl, or heterocyclylalkyl, each of which is independently unsubstituted or substituted with one or more R^{A}.

In some embodiments, the compound of Formula (I) has the structure of Formula (Ib), or a pharmaceutically acceptable salt thereof:

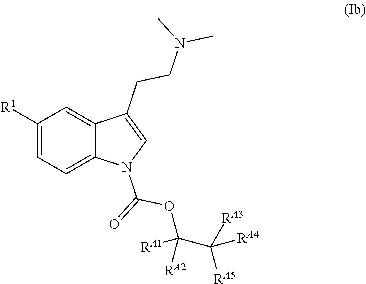

wherein:
R¹ is methoxy or hydrogen;
each of R^{A1}, R^{A2}, R^{A3}, and R^{A4} is independently hydrogen or alkyl that is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹, and R^{A5} is heteroalkyl, heterocyclylalkyl, heteroaryl, or —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each heteroalkyl, heterocyclylalkyl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (I) having the structure of Formula (Ib-1), or a pharmaceutically acceptable salt thereof:

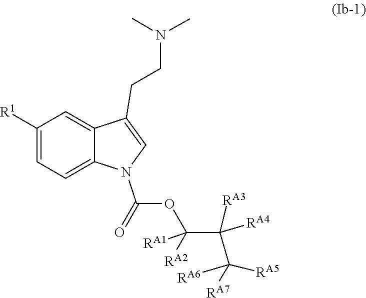

wherein:
R¹ is methoxy or hydrogen;
each of R^{A1}, R^{A2}, R^{A3}, R^{A4}, R^{A6}, and R^{A7} is independently hydrogen or alkyl that is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹, and R^{A5} is heteroalkyl, heterocyclylalkyl, heteroaryl, or —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each heteroalkyl, heterocyclylalkyl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) or (Ib) has the structure of Formula (Ib1), or a pharmaceutically acceptable salt thereof:

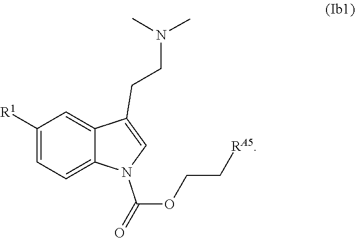

In some embodiments, the compound of Formula (I) having the structure of Formula (Ic), or a pharmaceutically acceptable salt thereof:

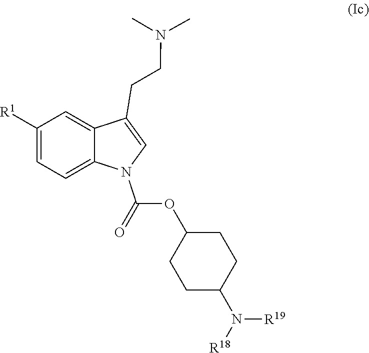

wherein R¹ is hydrogen or methoxy, and each of R¹⁸ and R¹⁹ is independently hydrogen, alkyl, cycloalkyl, or heteroalkyl, wherein each alkyl, cycloalkyl, and heteroalkyl is independently unsubstituted or substituted with one or more R^{B}; or R¹⁸ and R¹⁹ together with the atom to which they are attached form a heterocyclylalkyl ring that is unsubstituted or substituted with one or more R^{B}.

In some embodiments, the compound of Formula (I) having the structure of Formula (Id), or a pharmaceutically acceptable salt thereof:

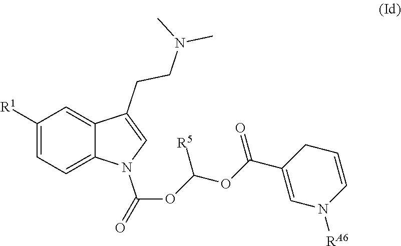

wherein R¹ is hydrogen or methoxy; R⁵ is alkyl or cycloalkyl, each of which is independently unsubstituted or substituted with one or more R^{A}, or hydrogen; and R^{A6} is hydrogen or alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) having the structure of Formula (Ie), or a pharmaceutically acceptable salt thereof:

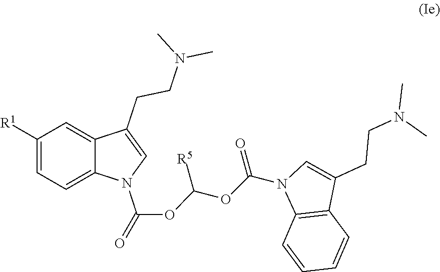

wherein R¹ is hydrogen or methoxy, and R⁵ is hydrogen, alkyl or cycloalkyl, wherein each alkyl and cycloalkyl is independently unsubstituted or substituted with one or more R^{A}.

In some embodiments, the compound of Formula (I) having the structure of Formula (If), or a pharmaceutically acceptable salt thereof:

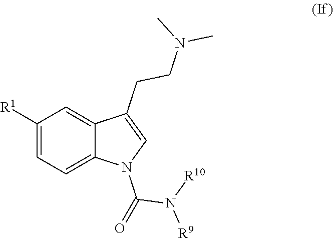

wherein R¹ is methoxy or hydrogen, and each of R⁹ and R¹⁰ is independently hydrogen, alkyl, cycloalkyl, aryl, heteroaryl, heteroalkyl, or heterocyclylalkyl, wherein each alkyl, cycloalkyl, aryl, heteroaryl, heteroalkyl, and heterocyclylalkyl is independently unsubstituted or substituted with one or more R^{A}, or R⁹ and R¹⁰ together with the atom to which they are attached form a heterocyclylalkyl ring that is unsubstituted or substituted with one or more R^{A}.

In some embodiments, the compound of Formula (I) or (If) having the structure of Formula (If1), or a pharmaceutically acceptable salt thereof:

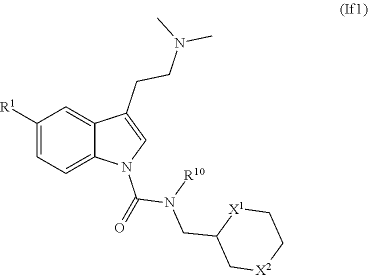

wherein:
R¹ is methoxy or hydrogen;
R¹⁰ is hydrogen, alkyl, heteroalkyl, cycloalkyl, or heterocyclylalkyl, wherein each of alkyl, heteroalkyl, cycloalkyl, and heterocyclylalkyl is unsubstituted or substituted with one or more R^{A}; and
each of X¹ and X² are independently selected from —CH₂—, —O—, —NH—, —S—, —S(O)—, —S(O)₂—, or —N(Y¹)—, wherein each Y¹ is independently hydrogen, cycloalkyl, heteroalkyl, or alkyl.

In some embodiments, the compound of Formula (I) having the structure of Formula (Ig), or a pharmaceutically acceptable salt thereof:

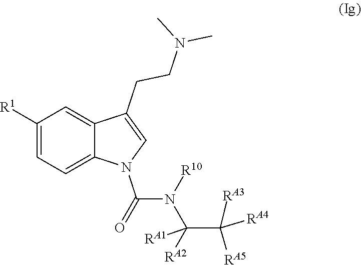

wherein:
R¹ is methoxy or hydrogen;
each of R^{A1}, R^{A2}, R^{A3}, and R^{A4} is independently hydrogen or alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R¹⁰ is hydrogen, alkyl, heteroalkyl, or cycloalkyl, wherein each of alkyl, heteroalkyl, and cycloalkyl is unsubstituted or substituted with one or more R^{A}; and R^{A5} is heteroalkyl, heterocyclylalkyl, heteroaryl, or —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each of heteroalkyl, heterocyclylalkyl, heteroaryl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) having the structure of Formula (Ih), or a pharmaceutically acceptable salt thereof:

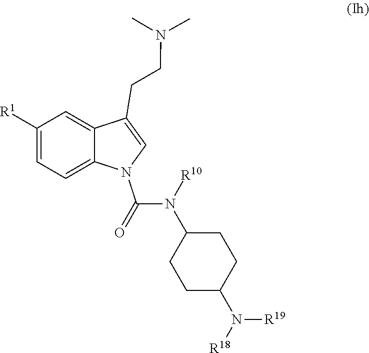

wherein:
R¹ is hydrogen or methoxy;
R¹⁰ is hydrogen, alkyl, heteroalkyl, or cycloalkyl, wherein each of alkyl, heteroalkyl, and cycloalkyl is unsubstituted or substituted with one or more R^{A}; and each of R¹⁸ and R¹⁹ is independently hydrogen, alkyl, cycloalkyl, or heteroalkyl, wherein each alkyl, cycloalkyl, or heterocyclylalkyl is independently unsubstituted or substituted with one or more R^{B}; or R¹⁸ and R¹⁹ together with the atom to which they are attached form a heterocyclylalkyl ring that is unsubstituted or substituted with one or more R^{B}.

In some embodiments, the compound of Formula (I) having the structure of Formula (Ii), or a pharmaceutically acceptable salt thereof:

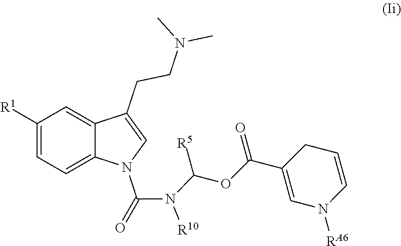

wherein:

R¹ is hydrogen or methoxy; and
each of R⁵ and R¹⁰ is independently hydrogen, alkyl, heteroalkyl, or cycloalkyl, wherein each alkyl, heteroalkyl, and cycloalkyl is independently unsubstituted or substituted with one or more R^{A}; and
R^{A6} is independently hydrogen, alkyl, heteroalkyl, or cycloalkyl, wherein each of alkyl, heteroalkyl, or cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) having the structure of Formula (Ij), or a pharmaceutically acceptable salt thereof:

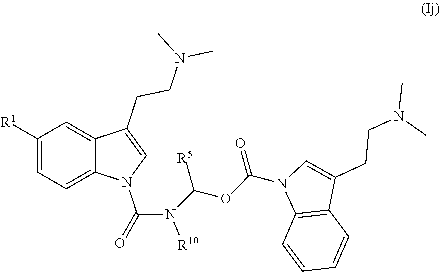

wherein R¹ is hydrogen or methoxy, and each of R⁵ and R¹⁰ is hydrogen, alkyl, or heteroalkyl, wherein each of alkyl and heteroalkyl is independently unsubstituted or substituted with one or more R^{A}.

In some embodiments, the compound of Formula (I) having the structure of Formula (Ik), or a pharmaceutically acceptable salt thereof:

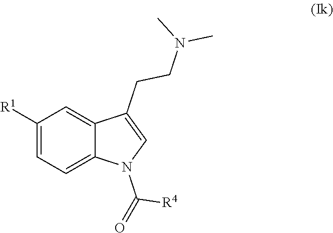

wherein R¹ is hydrogen or methoxy, and R⁴ is alkyl, heterocyclylalkyl, aryl, heteroaryl, or heteroalkyl, each of which is unsubstituted or substituted with one or more R^{A}.

In some embodiments, the compound of Formula (I) or (Ik) having the structure of Formula (Ik1), or a pharmaceutically acceptable salt thereof:

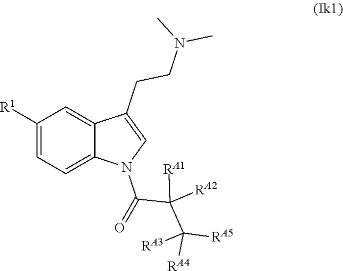

R¹ is methoxy or hydrogen;
each of R^{A1}, R^{A2}, R^{A3}, and R^{A4} is independently hydrogen, alkyl, or an amino acid side chain, wherein each alkyl or amino acid side chain is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R¹⁰ is hydrogen, alkyl, heteroalkyl, or cycloalkyl, wherein each of alkyl, heteroalkyl, and cycloalkyl is unsubstituted or substituted with one or more R^{A}; and
R^{A5} is heteroalkyl, heterocyclylalkyl, heteroaryl, —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each of heteroalkyl, heterocyclylalkyl, heteroaryl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) or (Ik) having the structure of Formula (Ik2), or a pharmaceutically acceptable salt thereof:

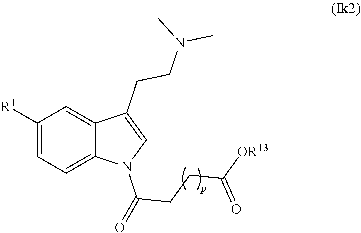

wherein:
R¹ is methoxy or hydrogen;
R¹³ is hydrogen, alkyl, cycloalkyl, aryl, heteroaryl, heteroalkyl, or heterocyclylalkyl, wherein each of alkyl, cycloalkyl, aryl, heteroaryl, heteroalkyl, and heterocyclylalkyl is unsubstituted or substituted with one or more R^{B}; and
p is 0, 1, 2, 3, 4, 5, 6, 7, 8, 9, or 10.

In some embodiments, the compound of Formula (I) or (Ik) having the structure of Formula (Ik3), or a pharmaceutically acceptable salt thereof:

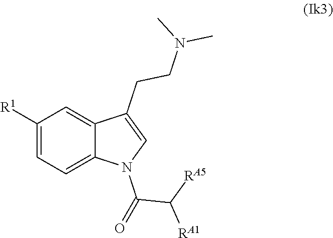

wherein:
R¹ is methoxy or hydrogen;
R^{A1} is alkyl or an amino acid side chain, each of which is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
R^{A5} is —N(R¹⁸)R¹⁹ or —N(R¹³)C(O)R¹⁴.

In some embodiments, the compound of Formula (I) having the structure of Formula (Il), or a pharmaceutically acceptable salt thereof:

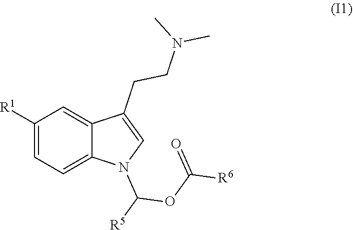

wherein:
R¹ is methoxy or hydrogen;
R⁵ is hydrogen, alkyl, or cycloalkyl, wherein each of alkyl or cycloalkyl is unsubstituted or substituted with one or more R^{A}; and
R⁶ is alkyl, cycloalkyl, heterocyclylalkyl, or heteroalkyl, wherein each of alkyl, cycloalkyl, heterocyclylalkyl, or heteroalkyl is unsubstituted or substituted with one or more R^{A}.

In some embodiments, the compound of Formula (I) having the structure of Formula (Im), or a pharmaceutically acceptable salt thereof:

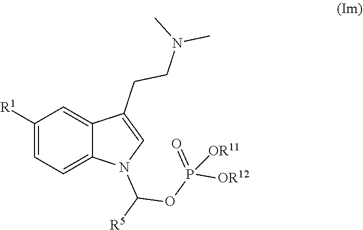

wherein:
R¹ is methoxy or hydrogen;
R⁵ hydrogen, alkyl, cycloalkyl, or heteroalkyl, wherein each of alkyl, cycloalkyl, and heteroalkyl is unsubstituted or substituted with one or more R^{A}; and
each of R¹¹ and R¹² is independently hydrogen, cycloalkyl, aryl, heteroaryl, or alkyl, wherein each of alkyl, cycloalkyl, and heteroalkyl is independently unsubstituted or substituted with one or more R^{A}.

In some embodiments, the compound of Formula (I) or (Im) having the structure of Formula (Im1), or a pharmaceutically acceptable salt thereof:

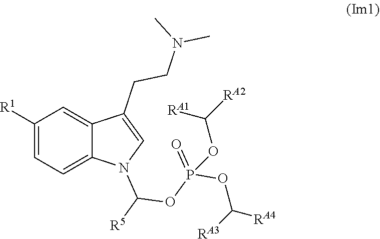

wherein:
R¹ is methoxy or hydrogen;
each of R^{A1}, R^{A3}, and R⁵ is independently hydrogen, alkyl, or cycloalkyl; and
each of R^{A2} and R^{A4} is independently alkyl, heteroalkyl, cycloalkyl, heterocyclylalkyl, aryl, or heteroaryl, —OC(O)R¹⁵, or —OC(O)OR¹⁶,
   wherein each alkyl, heteroalkyl, cycloalkyl, heterocyclylalkyl, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I), (Im), or (Im1) having the structure of Formula (Im1a), or a pharmaceutically acceptable salt thereof:

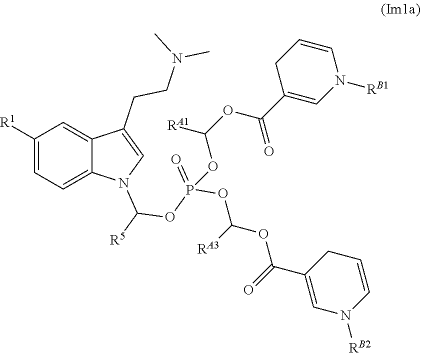

wherein:
R¹ is methoxy or hydrogen;
each of R^{A1}, R^{A3}, and R⁵ is independently hydrogen, alkyl, or cycloalkyl, wherein each of alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R^{B1} and R^{B2} is independently hydrogen or alkyl that is unsubstituted or substituted with one or more halogen, amino, cyano, hydroxyl, alkyl, acetyl, or benzoyl.

In some embodiments, the compound of Formula (I) having the structure of Formula (In), or a pharmaceutically acceptable salt thereof:

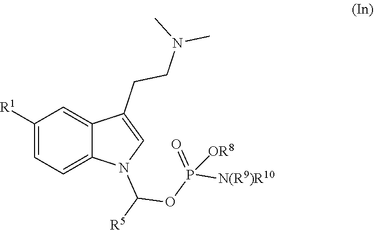

wherein:
R¹ is methoxy or hydrogen;
R⁵ is hydrogen, alkyl, or cycloalkyl;
R⁸ is hydrogen, alkyl, cycloalkyl, aryl, heterocyclylalkyl, or heteroaryl; and
each of R⁹ and R¹⁰ is independently hydrogen or alkyl,
   wherein each cycloalkyl, aryl, heterocyclylalkyl, and heteroaryl is independently unsubstituted or substituted with one or more R^{A}.

In some embodiments, the compound of Formula (I) or (In) having the structure of Formula (In1), or a pharmaceutically acceptable salt thereof:

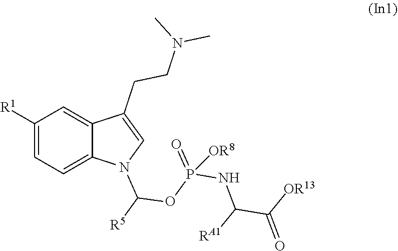

wherein:
R¹ is methoxy or hydrogen;
R^{A1} is hydrogen, alkyl, or cycloalkyl, wherein each of alkyl and cycloalkyl is unsubstituted or substituted with alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R⁵ and R⁸ is hydrogen, alkyl, cycloalkyl, aryl, heterocyclylalkyl, or heteroaryl, wherein alkyl, cycloalkyl, aryl, heterocyclylalkyl, and heteroaryl is independently unsubstituted or substituted with one or more R^{A}; and
R¹³ is hydrogen or alkyl that is unsubstituted or substituted with one or more R^{B}.

In some embodiments, the compound of Formula (I) having the structure of Formula (Io), or a pharmaceutically acceptable salt thereof:

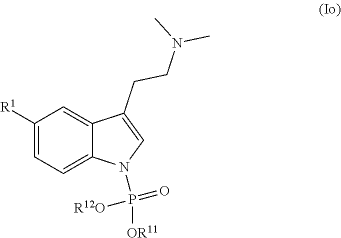

wherein:
R¹ is methoxy or hydrogen; and
each of R¹¹ and R¹² is independently selected from hydrogen, cycloalkyl, aryl, heteroaryl, or alkyl, wherein each cycloalkyl, aryl, heteroaryl, and alkyl is independently unsubstituted or substituted with one or more R^{A}, or R¹¹ and R¹² together with the atoms to which they are attached form a heterocyclylalkyl ring that is unsubstituted or substituted with one or more R^{A}.

In some embodiments, the compound of Formula (I) or (Io) having the structure of Formula (Io1), or a pharmaceutically acceptable salt thereof:

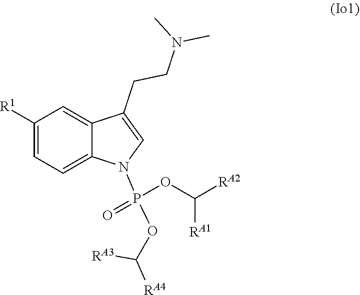

wherein:
R¹ is methoxy or hydrogen;
each of R^{A1} and R^{A3} is independently hydrogen, alkyl, or cycloalkyl; and
each of R^{A2} and R^{A4} is independently alkyl, heteroalkyl, cycloalkyl, heterocyclylalkyl, aryl, heteroaryl, —OC(O)R⁵, or —OC(O)OR¹⁶,
   wherein each alkyl, heteroalkyl, cycloalkyl, heterocyclylalkyl, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I) or (Io) having the structure of Formula (Io2), or a pharmaceutically acceptable salt thereof:

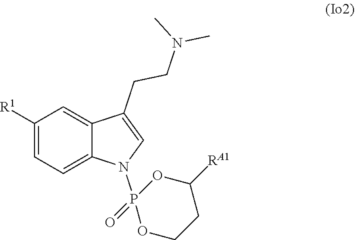

wherein R¹ is methoxy or hydrogen; and R^{A1} is aryl or heteroaryl, each of which is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, the compound of Formula (I), (Io), or (Io1), having the structure of Formula (Io1a), or a pharmaceutically acceptable salt thereof:

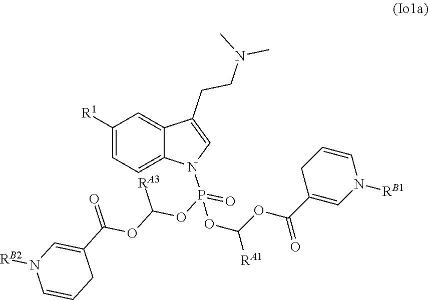

wherein:
R¹ is methoxy or hydrogen;
each of R^{A1} and R^{A3} is independently hydrogen, alkyl, or cycloalkyl, wherein each alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R^{B1} and R^{B2} is independently hydrogen or alkyl that is unsubstituted or substituted with one or more halogen, amino, cyano, hydroxyl, alkyl, acetyl, or benzoyl.

In some embodiments, the compound of Formula (I) having the structure of Formula (Ip), or a pharmaceutically acceptable salt thereof:

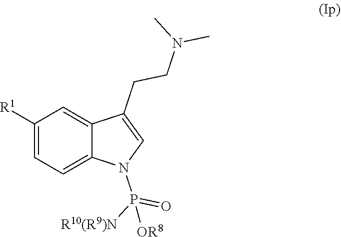

wherein:

R¹ is methoxy or hydrogen;
R⁸ is alkyl, cycloalkyl, aryl, heterocyclylalkyl, or heteroaryl; and
each of R⁹ and R¹⁰ is independently hydrogen or alkyl,
   wherein each alkyl, cycloalkyl, aryl, heterocyclylalkyl, and heteroaryl is independently unsubstituted or substituted with one or more R^{A}.

In some embodiments, the compound of Formula (I) or (Ip) having the structure of Formula (Ip1), or a pharmaceutically acceptable salt thereof:

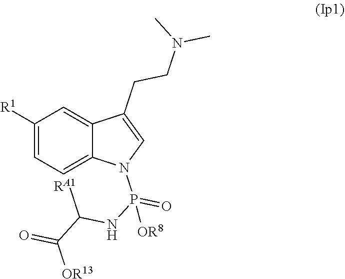

wherein:
R¹ is methoxy or hydrogen;
R^{A1} is hydrogen, alkyl, or cycloalkyl, wherein each of alkyl and cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R⁸ is hydrogen, alkyl, cycloalkyl, aryl, heterocyclylalkyl, or heteroaryl, wherein each alkyl, cycloalkyl, aryl, heterocyclylalkyl, and heteroaryl is unsubstituted or substituted with one or more R^{A}; and
R¹³ is hydrogen or alkyl that is unsubstituted or substituted with one or more R^{B}.

In some embodiments, the compound of Formula (I) having the structure of Formula (Iq), or a pharmaceutically acceptable salt thereof:

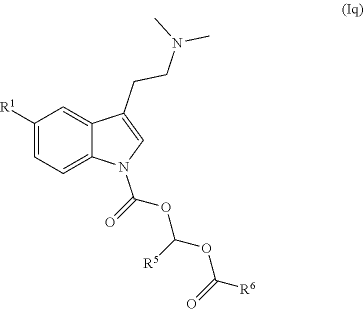

wherein:
R¹ is methoxy or hydrogen;
R⁵ is hydrogen, alkyl, or cycloalkyl; and
R⁶ is alkyl, cycloalkyl, heteroalkyl, heterocyclylalkyl, aryl, or heteroaryl,
   wherein each alkyl, cycloalkyl, heteroalkyl, heterocyclylalkyl, aryl, and heteroaryl is independently unsubstituted or substituted with one or more R^{A}.

In some embodiments, the compound of Formula (I) or (Iq) having the structure of Formula (Iq1), or a pharmaceutically acceptable salt thereof:

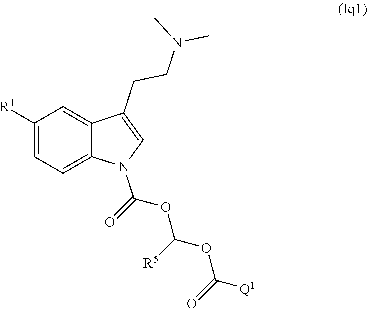

wherein:
R¹ is methoxy or hydrogen;
R⁵ is hydrogen, alkyl, or cycloalkyl, wherein each of alkyl and cycloalkyl is unsubstituted or substituted with one or more R^{A}; and
Q¹ is

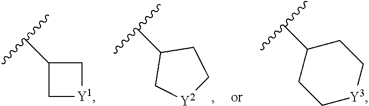

wherein
   each of Y¹, Y², or Y³ is independently —O—, —S—, —S(O)—, —S(O)₂—, —N(R^{Y1})—, or —NC(O)R^{Y2}, wherein each of R^{Y1} and R^{Y2} is independently hydrogen, alkyl, heteroalkyl, or heteroaryl.

In some embodiments, the compound of Formula (I) having the structure of Formula (Ir), or a pharmaceutically acceptable salt thereof:

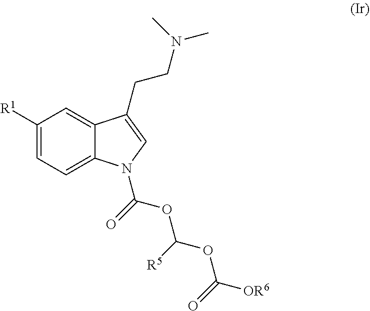

wherein:
R¹ is methoxy or hydrogen;
R⁵ is hydrogen, alkyl, or cycloalkyl; and
R⁶ is alkyl, cycloalkyl, heteroalkyl, heterocyclylalkyl, aryl, or heteroaryl,
   wherein each alkyl, cycloalkyl, heteroalkyl, heterocyclylalkyl, aryl, and heteroaryl is independently unsubstituted or substituted with one or more R^{A}.

In some embodiments, the compound of Formula (I) or (Ir) having the structure of Formula (Ir1), or a pharmaceutically acceptable salt thereof:

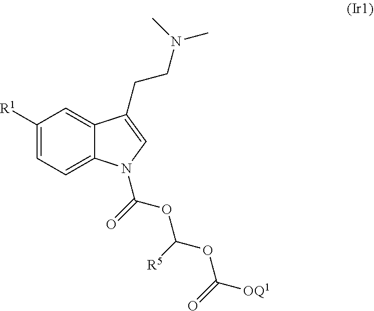

wherein:
R¹ is methoxy or hydrogen;
R⁵ is hydrogen, alkyl, or cycloalkyl, wherein each of alkyl and cycloalkyl is unsubstituted or substituted with one or more R^{A}; and
Q¹ is

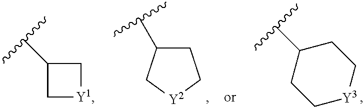

wherein
   each of Y¹, Y², or Y³ is independently —O—, —S—, —S(O)—, —S(O)₂—, —N(R^{Y1})—, or —NC(O)R^{Y2}, wherein each of R^{Y1} and R^{Y2} is independently hydrogen, alkyl, heteroalkyl, or heteroaryl.

In some embodiments, the compound of Formula (I) having the structure of Formula (Is), or a pharmaceutically acceptable salt thereof:

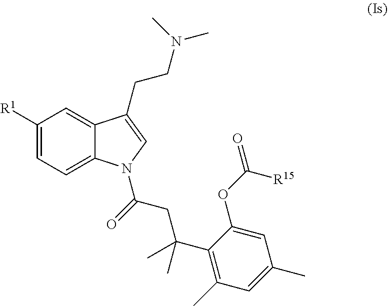

wherein R¹ is hydrogen or methoxy, and R¹⁵ is alkyl, heteroalkyl, cycloalkyl, aryl, or heteroaryl, each of which is unsubstituted or substituted with one or more R^{B}.

In some embodiments, the compound of Formula (I) having the structure of Formula (It), or a pharmaceutically acceptable salt thereof:

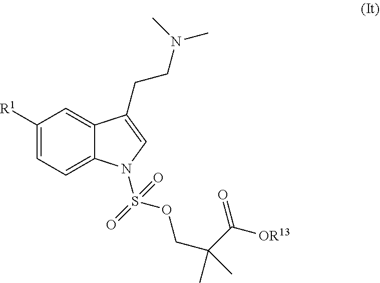

wherein R¹ is hydrogen or methoxy, and R¹³ is alkyl, cycloalkyl, heterocyclylalkyl, aryl, or heteroaryl, each of which is unsubstituted or substituted with one or more R^{B}.

In some embodiments, the compound of Formula (I) having the structure of Formula (Iu), or a pharmaceutically acceptable salt thereof:

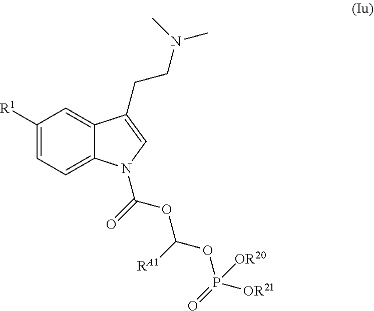

wherein:
R¹ is hydrogen or methoxy;
R^{A1} is hydrogen, alkyl, or cycloalkyl, wherein each of alkyl and cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R²⁰ and R²¹ is independently hydrogen, alkyl, cycloalkyl, aryl, heterocyclylalkyl, or heteroaryl, wherein each alkyl, cycloalkyl, aryl, heterocyclylalkyl, and heteroaryl is independently unsubstituted or substituted with one or more R^{B}, or R²⁰ and R²¹ together with the atoms to which they are attached form a heterocyclylalkyl ring that is unsubstituted or substituted with one or more R^{B}.

In some embodiments is a compound of Formula (I) having the structure of Formula (Iv), or a pharmaceutically acceptable salt thereof:

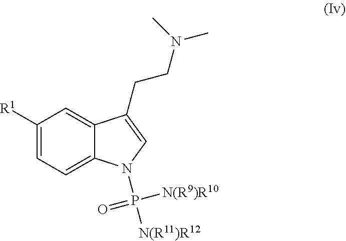

wherein:
R¹ is hydrogen or methoxy;
each of R⁹ and R¹⁰ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R⁹ and R¹⁰ together with the atom to which they are attached form a heterocyclylalkyl ring that is unsubstituted or substituted with one or more R^{A}; and
each of R¹¹ and R¹² is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R¹¹ and R¹² together with the atoms to which they are attached form a heterocyclylalkyl ring that is unsubstituted or substituted with one or more R^{A}In some embodiments, the compound of Formula (I) having the structure of Formula (Iw), or a pharmaceutically acceptable salt thereof:

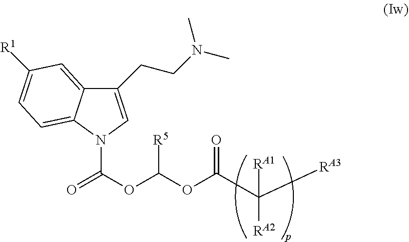

wherein:
R¹ is hydrogen or methoxy;
each R^{A1} and R^{A2} is independently hydrogen, alkyl, or cycloalkyl, wherein each alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R^{A3} is —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), and
p is 1, 2, 3, 4, 5, 6, 7, 8, 9, or 10.

In another aspect, the present disclosure provides a pharmaceutically acceptable composition comprising a compound according to any of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, and a pharmaceutically acceptable excipient, carrier, adjuvant, or vehicle.

In another aspect, the present disclosure provides a method of treating a condition in a subject in need thereof, the method comprising administering to the subject a therapeutically-effective amount of a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof.

### BRIEF DESCRIPTION OF THE FIGURES

FIG. 1 shows the mean concentration-time profiles of DMT following oral dosing of DMT to Male SD rats (1 mg/kg for IV dosing, and 10 mg/kg for oral dosing).

FIG. 2 shows the mean concentration-time profiles of DMT following oral dosing of 5-MeO-DMT to Male SD rats (1 mg/kg for IV dosing, and 10 mg/kg for oral dosing).

FIG. 3 depicts the time course of blood plasma concentrations of N,N-dimethyltryptamine (DMT) and corresponding prodrug Compound 20 in Sprague-Dawley rats that have been intravenously administered (IV) Compound 20 at 1 mg/kg (Panel A) or orally administered (PO) Compound 20 at 10 mg/kg (Panel B).

FIG. 4 shows the mean concentration-time profiles of DMT following IV or oral dosing of Compound 20 to Male SD rats (1 mg/kg for IV dosing, 10 mg/kg for oral dosing).

FIG. 5 shows the mean concentration-time profiles of Compound 20 following IV or oral dosing of Compound 20 to Male SD rats (1 mg/kg for IV dosing, 10 mg/kg for oral dosing).

FIG. 6 depicts the time course of blood plasma concentrations of 5-methoxy-N,N-dimethyltryptamine (5-MeO-DMT) and corresponding prodrug Compound 19 in Sprague-Dawley rats that have been intravenously administered (IV) Compound 19 at 1 mg/kg (Panel A) or orally administered (PO) Compound 19 at 10 mg/kg (Panel B).

FIG. 7 shows the mean concentration-time profiles of 5-methoxy-N,N-dimethyltryptamine (5-MeO-DMT) following IV or oral dosing of Compound 19 to Male SD rats (1 mg/kg for IV dosing, 10 mg/kg for oral dosing) are shown in.

FIG. 8 shows the Mean Total Concentrations of DMT following PO administration of DMT Prodrug to male Sprague Dawley rat at 10 mg/kg.

FIG. 9 shows the Mean Total Concentrations of DMT Prodrug following IV, PO administration to male Sprague Dawley rat at 1.10 mg/kg.

FIG. 10 shows the Mean Total concentrations of 5-MeO-DMT following PO administration of 5-MeO-DMT Pro-drug to male Sprague Dawley rat at 10 mg/kg.

FIG. 11 shows the Mean Total concentrations of 5-MeO-DMT Prodrug following IV, PO administration to male Sprague Dawley rat at 1.10 mg/kg.

FIG. 12 shows the Mean Concentration-Time Profiles of DMT CP-2 and Metabolite DMT Following Oral Dosing of DMT CP-2 (10 mg/Kg) to Male SD Rats.

FIG. 13 shows the Mean Concentration-Time Profiles of DMT CP-3 and the Metabolite DMT Following Oral Dosing of DMT CP-3 (10 mg/Kg) to Male SD Rats.

FIG. 14 shows the Mean Concentration-Time Profiles of DMT CP-4 and the Metabolite DMT Following Oral Dosing of DMT CP-4 (10 mg/Kg) to Male SD Rats.

FIG. 15 shows the Mean Concentration-Time Profiles of DMT CP-5 and the Metabolite DMT Following Oral Dosing of DMT CP-5 (10 mg/Kg) to Male SD Rats.

FIG. 16 shows the Mean Concentration-Time Profiles of DMT AP-1 and the Metabolite DMT Following Oral Dosing of DMT AP-1 (10 mg/Kg) to Male SD Rats.

FIG. 17 shows the Mean Concentration-Time Profiles of 5-MeO-DMT CP-2 and the Metabolite 5-MeO-DMT Following Oral Dosing of 5-MeO-DMT CP-2 (10 mg/Kg) to Male SD Rats.

FIG. 18 shows the Mean Concentration-Time Profiles of 5-MeO-DMT CP-3 and the Metabolite 5-MeO-DMT Following Oral Dosing of 5-MeO-DMT CP-3 (10 mg/Kg) to Male SD Rats.

FIG. 19 shows the Mean Concentration-Time Profiles of 5-MeO-DMT CP-4 and the Metabolite 5-MeO-DMT Following Oral Dosing of 5-MeO-DMT CP-4 (10 mg/Kg) to Male SD Rats.

FIG. 20 shows the Mean Concentration-Time Profiles of 5-MeO-DMT CP-5 and the Metabolite 5-MeO-DMT Following Oral Dosing of 5-MeO-DMT CP-5 (10 mg/Kg) to Male SD Rats.

FIG. 21 shows Mean Concentration-Time Profiles of 5-MeO-DMT AP-1 and the Metabolite 5-MeO-DMT Following Oral Dosing of 5-MeO-DMT AP-1 (10 mg/Kg) to Male SD Rats.

FIG. 22 shows the Mean Concentration-Time Profiles of DMT Benzamide and the Metabolite DMT Following Oral Dosing of DMT Benzamide (10 mg/Kg) to Male SD Rats.

FIG. 23 shows the Mean Concentration-Time Profiles of 5-MeO-DMT Prodrug and Metabolite 5-MeO-DMT Following Oral Dosing of 5-MeO-DMT Prodrug (10 mg/Kg) to Male SD Rats.

FIG. 24 shows the Mean Concentration-Time Profiles of 5-MeO-DMT Prodrug and Metabolite 5-MeO-DMT Following Oral Dosing of 5-MeO-DMT Prodrug (10 mg/Kg) to Male SD Rats.

FIG. 25 shows the Mean Concentration-Time Profiles of the Metabolite 5-MeO-DMT Following Oral Dosing of 5-MeO-DMT methylpivaloyl carbamate (10 mg/Kg) to Male SD Rats.

FIG. 26 shows the Mean Concentration-Time Profiles of the Metabolite DMT Following Oral Dosing of DMT methoxyethyl carbamate formate (10 mg/Kg) to Male SD Rats.

FIG. 27 shows the Mean Concentration-Time Profiles of the Metabolite 5-MeO-DMT Following Oral Dosing of 5-MeO-DMT methoxyethyl carbamate (10 mg/Kg) to Male SD Rats.

FIG. 28 shows the Mean Concentration-Time Profiles of the Metabolite DMT Following Oral Dosing of DMT trimethyl lock amide (10 mg/Kg) to Male SD Rats.

FIG. 29 shows the Mean Concentration-Time Profiles of the Metabolite 5-MeO-DMT Following Oral Dosing of 5-MeO-DMT trimethyl lock amide (10 mg/Kg) to Male SD Rats.

FIG. 30 shows the Mean Concentration-Time Profiles of Metabolite 5-MeO-DMT Following Oral Dosing of 5-MeO-DMT 4-Piperidinopiperidine urea formate (10 mg/Kg) to Male SD Rats.

FIG. 31 shows the Mean Concentration-Time Profiles of Metabolite 5-MeO-DMT Following Oral Dosing of the 5-MeO-DMT N,N-dimethyl urea formate prodrug of 5-MeO-DMT (10 mg/Kg) to Male SD Rats.

FIG. 32 shows the Mean Concentration-Time Profiles of Metabolite DMT Following Oral Dosing of the prodrug DMT Lysine ti-hydrochloride (10 mg/Kg) to Male SD Rats.

FIG. 33 shows the Mean Concentration-Time Profiles of Metabolite 5-MeO-DMT Following Oral Dosing of the prodrug 5-MeO-DMT Lysine tri-hydrochloride (10 mg/Kg) to Male SD Rats.

FIG. 34 shows the Mean Concentration-Time Profiles of Metabolite DMT Following Oral Dosing of the prodrug Di-DMT urea (symmetrical urea) di-formate salt (10 mg/Kg) to Male SD Rats.

FIG. 35 shows the Mean Concentration-Time Profiles of Metabolite 5-MeO-DMT Following Oral Dosing of the prodrug Di-5-MeO-DMT urea (symmetrical urea) di-formate salt (10 mg/Kg) to Male SD Rats.

FIG. 36 shows the Mean Concentration-Time Profiles of Metabolite DMT Following Oral Dosing of the prodrug DMT Valine di-hydrochloride (10 mg/Kg) to Male SD Rats.

FIG. 37 shows the Mean Concentration-Time Profiles of Metabolite 5-MeO-DMT Following Oral Dosing of the prodrug 5-MeO-DMT Valine di-hydrochloride (10 mg/Kg) to Male SD Rats.

FIG. 38 shows the Mean Concentration-Time Profiles of Metabolite 5-MeO-DMT Following Oral Dosing of the prodrug 5-MeO-DMT N,N-dimethylglycine formate (10 mg/Kg) to Male SD Rats.

FIG. 39 shows the Mean Concentration-Time Profiles of Metabolite DMT Following Oral Dosing of the prodrug Phe-N-Me-Gly DMT di-hydrochloride (DMT dipeptide) (10 mg/Kg) to Male SD Rats.

FIG. 40 shows the Mean Concentration-Time Profiles of Metabolite DMT Following Oral Dosing of the prodrug DMT Alanine di-hydrochloride (10 mg/Kg) to Male SD Rats.

FIG. 41 shows the Mean Concentration-Time Profiles of Metabolite 5-MeO-DMT Following Oral Dosing of the prodrug 5-MeO-DMT Alanine di-hydrochloride (10 mg/Kg) to Male SD Rats.

FIG. 42 shows the Mean Concentration-Time Profiles of Metabolite DMT Following Oral Dosing of the prodrug DMT tetramethylphosphorodiamide (10 mg/Kg) to Male SD Rats.

FIG. 43 shows Mean Concentration-Time Profiles of Metabolite 5-MeO-DMT Following Oral Dosing of the prodrug 5-MeO-DMT tetramethylphosphorodiamide (10 mg/Kg) to Male SD Rats.

FIG. 44 shows the Mean Concentration-Time Profiles of Metabolite DMT Following Oral Dosing of the prodrug DMT Phenylalanine di-hydrochloride (10 mg/Kg) to Male SD Rats.

FIG. 45 shows the Mean Concentration-Time Profiles of Metabolite 5-MeO-DMT Following Oral Dosing of the prodrug 5-MeO-DMT Phenylalanine di-hydrochloride (10 mg/Kg) to Male SD Rats.

FIG. 46 shows the Mean Concentration-Time Profiles of Metabolite 5-MeO-DMT Following Oral Dosing of the prodrug 5-MeO-DMT 2,2-dimethylpropyl pivalate carbamate formate (10 mg/Kg) to Male SD Rats.

FIG. 47 shows the Mean Concentration-Time Profiles of Metabolite DMT Following Oral Dosing of the prodrug DMT N,N-dimethylglycine hydrochloride (10 mg/Kg) to Male SD Rats.

FIG. 48 shows the Mean Concentration-Time Profiles of Metabolite DMT Following Oral Dosing of the prodrug DMT methyl pivalate (10 mg/Kg) to Male SD Rats.

FIG. 49 shows the Mean Concentration-Time Profiles of Metabolite 5-MeO-DMT Following Oral Dosing of the prodrug 5-MeO-DMT methyl pivalate (10 mg/Kg) to Male SD Rats.

FIG. 50 shows the Mean Concentration-Time Profiles of Metabolite 5-MeO-DMT Following Oral Dosing of the prodrug 5-MeO-DMT-3,3-dimethylsuccinate hydrochloride (10 mg/Kg) to Male SD Rats.

FIG. 51 shows the Mean Concentration-Time Profiles of Metabolite DMT Following Oral Dosing of the prodrug DMT 2,2-dimethylpropyl pivalate carbamate formate (10 mg/Kg) to Male SD Rats.

FIG. 52 shows the Mean Concentration-Time Profiles of Metabolite DMT Following Oral Dosing of the prodrug DMT methyl alcohol (10 mg/Kg) to Male SD Rats.

FIG. 53 shows the Mean Concentration-Time Profiles of Metabolite 5-MeO-DMT Following Oral Dosing of the prodrug 5-MeO-DMT methyl alcohol (10 mg/Kg) to Male SD Rats.

FIG. 54 shows the Mean Concentration-Time Profiles of Metabolite 5-MeO-DMT Following Oral Dosing of the prodrug 5-MeO-DMT carboxy-isopropyl valinate di-trifluoroacetate (10 mg/Kg) to Male SD Rats.

FIG. 55 shows the Mean Concentration-Time Profiles of Metabolite DMT Following Oral Dosing of the prodrug DMT methyl succinate (10 mg/Kg) to Male SD Rats.

FIG. 56 shows the Mean Concentration-Time Profiles of Metabolite 5-MeO-DMT Following Oral Dosing of the prodrug 5-MeO-DMT methyl succinate (10 mg/Kg) to Male SD Rats.

FIG. 57 shows the Mean Concentration-Time Profiles of Metabolite DMT Following Oral Dosing of the prodrug DMT methylpivaloyl carbamate formate (10 mg/Kg) to Male SD Rats.

FIG. 58 shows the Mean Concentration-Time Profiles of Metabolite DMT Following Oral Dosing of the Glutarate prodrug of DMT (10 mg/Kg) to Male SD Rats.

### DETAILED DESCRIPTION

Described herein are compounds that can be metabolically converted to N,N-dimethyltryptamine or analogs thereof upon administration to a subject. A compound disclosed herein can be useful for the treatment of a neurological disease, such as a psychiatric disorder, a substance abuse disorder, or a condition where increasing neuronal plasticity would be beneficial.

### Definitions

Compounds herein can include all stereoisomers, enantiomers, diastereomers, mixtures, racemates, atropisomers, and tautomers thereof.

Unless otherwise specified, any compound disclosed herein can be substituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclylalkyl groups, heteroaryl groups, cycloalkyl groups, acyl groups, acyloxy groups, carbamate groups, amide groups, ureido groups, epoxy groups, and ester groups.

Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Alkyl groups can include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl groups include straight, branched, and cyclic alkynyl groups. The triple bond of an alkylnyl group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-TH-azepinyl, 2,3-dihydro-TH-indole, and 1,2,3,4-tetrahydroquinoline; and ii) heterocyclic units having 2 or more rings one of which is a heterocyclic ring, non-limiting examples of which include hexahydro-TH-pyrrolizinyl, 3a,4,5,6,7,7a-hexahydro-TH-benzo[d]imidazolyl, 3a,4,5,6,7,7a-hexahydro-1H-indolyl, 1,2,3,4-tetrahydroquinolinyl, and decahydro-TH-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocyclylalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocyclylalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocyclylalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocyclylalkyl radical may be a monocyclic, bicyclic, tricyclic, or tetracyclic ring system, which may include fused (when fused with an aryl or a heteroaryl ring, the heterocyclylalkyl is bonded through a non-aromatic ring atom) or bridged ring systems; and the nitrogen, carbon, or sulfur atoms in the heterocyclylalkyl radical may be optionally oxidized; the nitrogen atom may be optionally quaternized.

Representative heterocyclylalkyls include, but are not limited to, heterocyclylalkyls having from two to fifteen carbon atoms (C₂-C₁₅ heterocyclylalkyl), from two to ten carbon atoms (C₂-C₁₀ heterocyclylalkyl), from two to eight carbon atoms (C₂-C₈ heterocyclylalkyl), from two to six carbon atoms (C₂-C₆ heterocyclylalkyl), from two to five carbon atoms (C₂-C₅ heterocyclylalkyl), or two to four carbon atoms (C₂-C₄ heterocyclylalkyl). In some embodiments, the heterocyclylalkyl is a 3- to 6-membered heterocyclylalkyl. In some embodiments, the cycloalkyl is a 5- to 6-membered heterocyclylalkyl. Examples of such heterocyclylalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocyclylalkyl also includes all ring forms of the carbohydrates, including but not limited to, the monosaccharides, the disaccharides, and the oligosaccharides. It is understood that when referring to the number of carbon atoms in a heterocyclylalkyl, the number of carbon atoms in the heterocyclylalkyl is not the same as the total number of atoms (including the heteroatoms) that make up the heterocyclylalkyl (i.e. skeletal atoms of the heterocyclylalkyl ring). Unless stated otherwise specifically in the specification, a heterocyclylalkyl is optionally substituted, for example, with oxo, halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heterocyclylalkyl is optionally substituted with oxo, halogen, methyl, ethyl, —CN, —CF₃, —OH, —OMe, —NH₂, or —NO₂. In some embodiments, a heterocyclylalkyl is optionally substituted with oxo, halogen, methyl, ethyl, —CN, —CF₃, —OH, or —OMe. In some embodiments, the heterocyclylalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocyclylalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocyclylalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Pharmaceutical Compositions.

According to another embodiment, the present disclosure provides a composition comprising a compound of the present disclosure and a pharmaceutically acceptable carrier, adjuvant, or vehicle. The amount of compound in the composition is an amount effective to treat the relevant disease, disorder, or condition in a patient in need thereof (an “effective amount”). In some embodiments, a composition of the present disclosure is formulated for oral administration to a patient.

The term “pharmaceutically acceptable carrier, adjuvant, or vehicle” refers to a non-toxic carrier, adjuvant, or vehicle that does not destroy the pharmacological activity of the agent with which it is formulated. Pharmaceutically acceptable carriers, adjuvants or vehicles that may be used in the disclosed compositions include, but are not limited to, ion exchangers, alumina, stearates such as aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally, enterally, intracistemally, intraperitoneally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the composition is administered orally, intraperitoneally, or intravenously. In some embodiments, the composition is a transmucosal formulation. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

To aid in delivery of the composition, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and com starch. Lubricating agents, such as magnesium stearate, may also be added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In some embodiments, the pharmaceutically acceptable composition is formulated for oral administration. Such formulations may be administered with or without food. In some embodiments, the pharmaceutically acceptable composition is administered without food. In other embodiments, the pharmaceutically acceptable composition is administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it can be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing a compound of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinyl pyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

Therapeutic agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Compounds of the Disclosure.

Described herein are compounds that can be metabolically converted to N,N-dimethyltryptamine or analogs thereof upon administration to a subject. In certain embodiments, the compounds described herein are useful in the treatment of conditions associated with any brain disease.

In some embodiments, the compounds described herein are prodrugs of dimethyltryptamine (DMT) or prodrugs of 5-MeO-DMT. In some embodiments, the compounds described herein are psychedelics with improved pharmacokinetic properties as compared to DMT or 5-MeO-DMT (e.g., longer half life, longer tmax, and/or longer tlast, etc.).

In one aspect, the present disclosure provides a compound of Formula (I), or a pharmaceutically acceptable salt thereof:

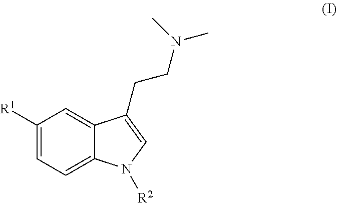

wherein:
R¹ is methoxy or hydrogen;
R² is —C(O)OR³, —C(O)R⁴, —CH(R⁵)OR⁶, —C(O)OCH(R⁵)OC(O)R⁶, —C(O)OCH(R⁵)OC(O)OR⁶, —CH(R⁵)C(O)R⁶, —CH(R⁵)OC(O)R⁶, —CH(R⁵)OC(O)OR⁶, —S(O)₂OR⁷, —P(O)OR⁸[N(R⁹)R¹⁰], —P(O)[N(R⁹)R¹⁰][N(R¹¹)R¹²], —C(O)N(R⁹)R¹⁰, —P(O)OR¹¹(OR¹²), —CH(R⁵)OP(O)OR⁹[N(R⁹)R¹⁰], or —CH(R⁵)OP(O)OR¹¹(OR¹²);
each of R³, R⁴, R⁵, R⁶, R⁷, and R⁸ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently unsubstituted or substituted with one or more R^{A}; each of R⁹ and R¹⁰ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R⁹ and R¹⁰ together with the atom to which they are attached form a heterocyclylalkyl ring that is unsubstituted or substituted with one or more R^{A};
each of R¹¹ and R¹² is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R¹¹ and R¹² together with the atoms to which they are attached form a heterocyclylalkyl ring that is unsubstituted or substituted with one or more R^{A};
each R^{A} is independently alkyl, heteroalkyl, cycloalkyl, heterocyclylalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹]—C(O)N(R¹⁸)R¹⁹, —OC(O)N(R¹⁸)R¹⁹, or —OP(O)OR²⁰(OR²¹), wherein each alkyl, heteroalkyl, cycloalkyl, heterocyclylalkyl, amino acid side chain, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B};
each of R¹⁸ and R¹⁹ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B}; or R¹⁸ and R¹⁹ together with the atom to which they are attached form a heterocyclylalkyl ring that is unsubstituted or substituted with one or more R^{B};
each of R²⁰ and R²¹ is independently alkyl, alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{B}, or R²⁰ and R²¹ together with the atoms to which they are attached form a heterocyclylalkyl ring that is unsubstituted or substituted with one or more R^{B}; and
each R^{B} is independently halogen, amino, cyano, hydroxyl, alkyl, heteroalkyl, cycloalkyl, heterocyclylalkyl, aryl, heteroaryl, arylalkyl, —C(O)CH₃, —C(O)Ph, or heteroarylalkyl, wherein each cycloalkyl, heterocyclylalkyl, aryl, and heteroaryl is independently unsubstituted or substituted with one or more halogen, amino, cyano, hydroxyl, alkyl, acetyl, or benzoyl.

In some embodiments is a compound of Formula (I) or a pharmaceutically acceptable salt thereof, wherein R² is R² is —C(O)OR³, —C(O)R⁴, —CH(R⁵)OR⁶, —C(O)OCH(R⁵)OC(O)R⁶, —C(O)OCH(R⁵)OC(O)OR⁶, —CH(R⁵)C(O)R⁶, —S(O)₂OR⁷, —P(O)OR⁸[N(R⁹)R¹⁰], —C(O)N(R⁹)R¹⁰, —P(O)OR¹¹(OR¹²), —CH(R⁵)OP(O)OR⁹[N(R⁹)R¹⁰], or —CH(R⁵)OP(O)OR¹¹(OR¹²).

In some embodiments is a compound of Formula (I) or a pharmaceutically acceptable salt thereof, wherein: each of R³, R⁴, R⁶, R⁷, and R⁸ is independently alkyl, heteroalkyl, cycloalkyl, heterocyclylalkyl, aryl, or heteroaryl; and each R⁵ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently unsubstituted or substituted with one or more R^{A}.

In some embodiments is a compound of Formula (I) or a pharmaceutically acceptable salt thereof, each of R³, R⁴, R⁵, R⁶, R⁷, and R⁸ is independently alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently unsubstituted or substituted with one or more R^{A}.

In some embodiments is a compound of Formula (I) or a pharmaceutically acceptable salt thereof, wherein R² is —C(O)OR³. In some embodiments is a compound of Formula (I) or a pharmaceutically acceptable salt thereof, wherein R² is —C(O)OR³, wherein R³ is alkyl. In some embodiments is a compound of Formula (I) or a pharmaceutically acceptable salt thereof, wherein R² is —C(O)OR³, wherein R³ is alkyl that is unsubstituted. In some embodiments is a compound of Formula (I) or a pharmaceutically acceptable salt thereof, wherein R² is —C(O)OR³, wherein R³ is heteroalkyl. In some embodiments is a compound of Formula (I) or a pharmaceutically acceptable salt thereof, wherein R² is —C(O)OR³, wherein R³ is heteroalkyl that is unsubstituted. In some embodiments is a compound of Formula (I) or a pharmaceutically acceptable salt thereof, wherein R² is —C(O)OR³, wherein R³ is ethyl.

In some embodiments is a compound of Formula (I) or a pharmaceutically acceptable salt thereof, wherein R² is —C(O)OR³, wherein R³ is alkyl. In some embodiments is a compound of Formula (I) or a pharmaceutically acceptable salt thereof, wherein R² is —C(O)OR³, wherein R³ is alkyl substituted with heterocyclylalkyl. In some embodiments is a compound of Formula (I) or a pharmaceutically acceptable salt thereof, wherein R² is —C(O)OR³, wherein R³ is alkyl substituted with —N(R¹³)C(O)OR¹⁴. In some embodiments is a compound of Formula (I) or a pharmaceutically acceptable salt thereof, wherein R¹³ is hydrogen or alkyl. In some embodiments is a compound of Formula (I) or a pharmaceutically acceptable salt thereof, wherein R¹⁴ is alkyl, aryl, or heteroaryl.

In some embodiments is a compound of Formula (I) or a pharmaceutically acceptable salt thereof, wherein R² is —C(O)OR³, wherein R³ is heteroalkyl. In some embodiments is a compound of Formula (I) or a pharmaceutically acceptable salt thereof, wherein R² is —C(O)OR³, wherein R³ is heteroalkyl that is substituted with cycloalkyl. In some embodiments is a compound of Formula (I) or a pharmaceutically acceptable salt thereof, wherein R² is —C(O)OR³, wherein R³ is heteroalkyl that is substituted with alkyl.

In some embodiments is a compound of Formula (I) or a pharmaceutically acceptable salt thereof, wherein R² is —C(O)OR³, wherein R³ is cycloalkyl. In some embodiments is a compound of Formula (I) or a pharmaceutically acceptable salt thereof, wherein R² is —C(O)OR³, wherein R³ is cycloalkyl that is substituted with N(R¹⁸)R¹⁹. In some embodiments is a compound of Formula (I) or a pharmaceutically acceptable salt thereof, wherein each of R¹⁸ and R¹⁹ is hydrogen, alkyl, or heteroalkyl. In some embodiments is a compound of Formula (I) or a pharmaceutically acceptable salt thereof, wherein R¹⁸ and R¹⁹ together with the atom to which they are attached form a heterocyclylalkyl ring. In some embodiments is a compound of Formula (I) or a pharmaceutically acceptable salt thereof, wherein R¹⁸ and R¹⁹ together with the atom to which they are attached form a heterocyclylalkyl ring. In some embodiments is a compound of Formula (I) or a pharmaceutically acceptable salt thereof, wherein R¹⁸ and R¹⁹ together with the atom to which they are attached form a heterocyclylalkyl ring that is unsubstituted.

In some embodiments is a compound of Formula (I) or a pharmaceutically acceptable salt thereof, wherein R² is —C(O)OR³, wherein R³ is alkyl. In some embodiments is a compound of Formula (I) or a pharmaceutically acceptable salt thereof, wherein R² is —C(O)OR³, wherein R³ is alkyl substituted with C(O)R¹⁴, wherein R¹⁴ is heteroaryl substituted with one or more R^{B}. In some embodiments is a compound of Formula (I) or a pharmaceutically acceptable salt thereof, wherein R² is —C(O)OR³, wherein R³ is alkyl substituted with C(O)R¹⁴, wherein R¹⁴ is heteroaryl. In some embodiments is a compound of Formula (I) or a pharmaceutically acceptable salt thereof, wherein R² is —C(O)OR³, wherein R³ is alkyl substituted with C(O)R¹⁴, wherein R¹⁴ is heterocyclylalkyl. In some embodiments is a compound of Formula (I) or a pharmaceutically acceptable salt thereof, wherein R² is —C(O)OR³, wherein R³ is alkyl substituted with C(O)R¹⁴, wherein R¹⁴ is heteroaryl that is unsubstituted. In some embodiments is a compound of Formula (I) or a pharmaceutically acceptable salt thereof, wherein R² is —C(O)OR³, wherein R³ is alkyl substituted with C(O)R¹⁴, wherein R¹⁴ is heterocyclylalkyl that is unsubstituted.

In some embodiments is a compound of Formula (I) or a pharmaceutically acceptable salt thereof, wherein R² is —C(O)R⁴, wherein R⁴ is heterocyclylalkyl.

In some embodiments is a compound of Formula (I) or a pharmaceutically acceptable salt thereof, wherein R² is —C(O)N(R⁹)R¹⁰. In some embodiments is a compound of Formula (I) or a pharmaceutically acceptable salt thereof, R⁴ and R⁵ together with the atom to which they are attached form a heterocyclylalkyl ring that is unsubstituted or substituted with one or more R^{A}. In some embodiments is a compound of Formula (I) or a pharmaceutically acceptable salt thereof, R⁴ and R⁵ together with the atom to which they are attached form a heterocyclylalkyl ring that is substituted with one or more R^{A}.

In some embodiments is a compound of Formula (I) having the structure of Formula (Ia), or a pharmaceutically acceptable salt thereof.

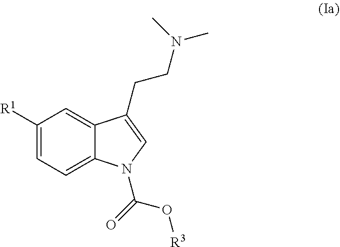

wherein R¹ is methoxy or hydrogen, and R³ is alkyl, cycloalkyl, aryl, heteroaryl, heteroalkyl, or heterocyclylalkyl, each of which is independently unsubstituted or substituted with one or more R^{A}. In some embodiments is a compound of Formula (I) or (Ia), or a pharmaceutically acceptable salt thereof, wherein R³ is alkyl or heteroalkyl. In some embodiments is a compound of Formula (I) or (Ia), or a pharmaceutically acceptable salt thereof, wherein R³ is unsubstituted alkyl or unsubstituted heteroalkyl. In some embodiments is a compound of Formula (I) or (Ia), or a pharmaceutically acceptable salt thereof, wherein R³ is alkyl. In some embodiments is a compound of Formula (I) or (Ia), or a pharmaceutically acceptable salt thereof, wherein R³ is unsubstituted alkyl. In some embodiments is a compound of Formula (I) or (Ia), or a pharmaceutically acceptable salt thereof, wherein R¹ is methoxy, and R³ is alkyl. In some embodiments is a compound of Formula (I) or (Ia), or a pharmaceutically acceptable salt thereof, wherein R¹ is methoxy, and R³ is unsubstituted alkyl. In some embodiments is a compound of Formula (I) or (Ia), or a pharmaceutically acceptable salt thereof, wherein R¹ is hydrogen, and R³ is alkyl. In some embodiments is a compound of Formula (I) or (Ia), or a pharmaceutically acceptable salt thereof, wherein R¹ is hydrogen, and R³ is unsubstituted alkyl.

In some embodiments is a compound of Formula (I) or (Ia), or a pharmaceutically acceptable salt thereof, wherein R³ is heteroalkyl. In some embodiments is a compound of Formula (I) or (Ia), or a pharmaceutically acceptable salt thereof, wherein R³ is unsubstituted heteroalkyl. In some embodiments is a compound of Formula (I) or (Ia), or a pharmaceutically acceptable salt thereof, wherein R³ is methyl, ethyl, n-propyl, isopropyl, n-butyl, tert-butyl, isobutyl, n-pentyl, or 3-methyl-1-butyl. In some embodiments is a compound of Formula (I) or (Ia), or a pharmaceutically acceptable salt thereof, wherein R³ is methyl, ethyl, n-propyl, isopropyl, n-butyl, isobutyl, n-pentyl, or 3-methyl-1-butyl. In some embodiments is a compound of Formula (I) or (Ia), or a pharmaceutically acceptable salt thereof, wherein R³ is aryl. In some embodiments is a compound of Formula (I) or (Ia), or a pharmaceutically acceptable salt thereof, wherein R³ is phenyl. In some embodiments is a compound of Formula (I) or (Ia), or a pharmaceutically acceptable salt thereof, wherein R³ is heterocyclylalkyl. In some embodiments is a compound of Formula (I) or (Ia), or a pharmaceutically acceptable salt thereof, wherein R³ is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (Ia), or a pharmaceutically acceptable salt thereof, wherein R³ is ethyl. In some embodiments is a compound of Formula (I) or (Ia), or a pharmaceutically acceptable salt thereof, wherein R¹ is hydrogen, and R³ is ethyl. In some embodiments is a compound of Formula (I) or (Ia), or a pharmaceutically acceptable salt thereof, wherein R¹ is methoxy, and R³ is ethyl. In some embodiments is a compound of Formula (I) or (Ia), or a pharmaceutically acceptable salt thereof, wherein R³ alkyl substituted with heteroaryl. In some embodiments is a compound of Formula (I) or (Ia), or a pharmaceutically acceptable salt thereof, wherein R³ is

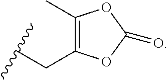 [SMILES: *Cc1oc(=O)oc1C]

In some embodiments is a compound of Formula (I) or (Ia), or a pharmaceutically acceptable salt thereof, wherein R¹ is methoxy and R³ is

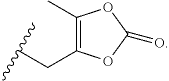 [SMILES: *Cc1oc(=O)oc1C]

In some embodiments is a compound of Formula (I) or (Ia), or a pharmaceutically acceptable salt thereof, wherein R¹ is hydrogen and R³ is

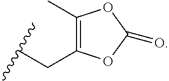 [SMILES: *Cc1oc(=O)oc1C]

In some embodiments is a compound of Formula (I) or (Ia), or a pharmaceutically acceptable salt thereof, wherein the compound is:

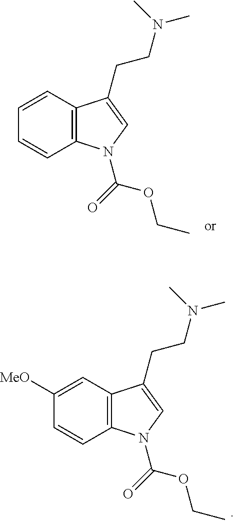 [SMILES: CCOC(=O)n1cc(CCN(C)C)c2ccccc21 ; CCOC(=O)n1cc(CCN(C)C)c2cc(OC)ccc21]

In some embodiments is a compound of Formula (I) or (Ia), or a pharmaceutically acceptable salt thereof, wherein the compound is:

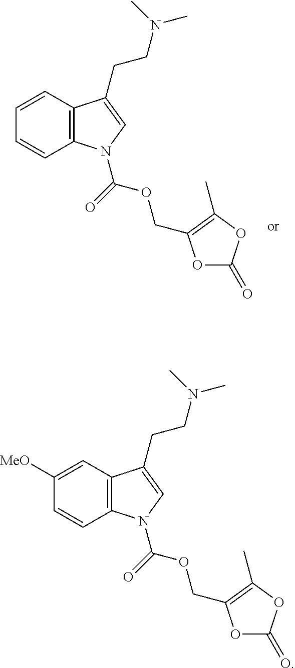 [SMILES: Cc1oc(=O)oc1COC(=O)n1cc(CCN(C)C)c2ccccc21 ; COc1ccc2c(c1)c(CCN(C)C)cn2C(=O)OCc1oc(=O)oc1C]

In some embodiments is a compound of Formula (I) or (Ia), or a pharmaceutically acceptable salt thereof, wherein the compound is:

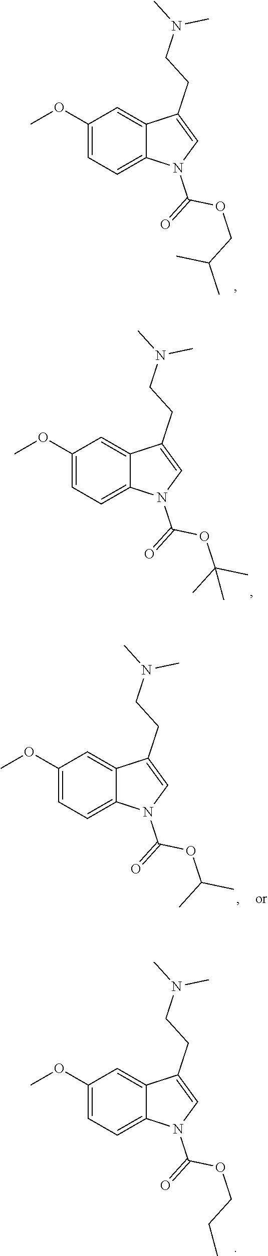

In some embodiments is a compound of Formula (I) or (Ia), or a pharmaceutically acceptable salt thereof, wherein the compound is:

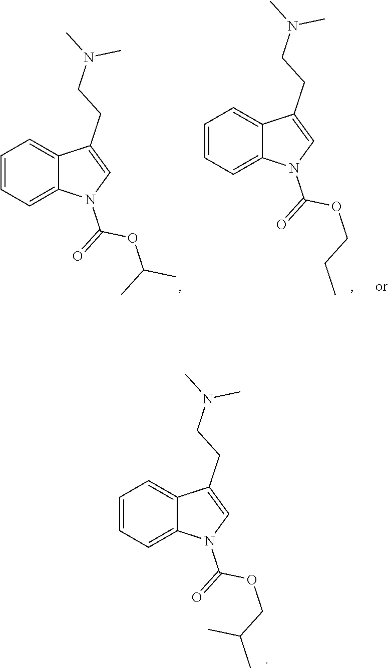 [SMILES: CC(C)OC(=O)n1cc(CCN(C)C)c2ccccc21 ; CCCOC(=O)n1cc(CCN(C)C)c2ccccc21 ; CC(C)COC(=O)n1cc(CCN(C)C)c2ccccc21]

In some embodiments is a compound of Formula (I) or (Ia), or a pharmaceutically acceptable salt thereof, provided that when R¹ is hydrogen, then R³ is not tert-butyl. In some embodiments is a compound of Formula (I) or (Ia), or a pharmaceutically acceptable salt thereof, wherein if R¹ is hydrogen and R³ is alkyl, then R³ is bound to the atom to which it is attached via a primary or secondary carbon.

In some embodiments is a compound of Formula (I) having the structure of Formula (Ib), or a pharmaceutically acceptable salt thereof:

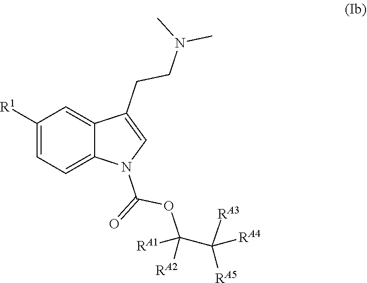

wherein:
R¹ is methoxy or hydrogen; each of R^{A1}, R^{A2}, R^{A3}, and R^{A4} is independently hydrogen or alkyl that is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹, and R^{A5} is heteroalkyl, heterocyclylalkyl, heteroaryl, or —C(O)OR³, —N(R¹³)C(O)OR¹⁴, —N(R¹³)C(O)R¹⁴, —C(O)R¹⁴, —OC(O)R¹⁵, or —OC(O)OR¹⁶, wherein each heteroalkyl, heterocyclylalkyl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Ib) or a pharmaceutically acceptable salt thereof, wherein one of R^{A1}, R^{A2}, R^{A3}, and R^{A4} is alkyl, and each of R^{A1}, R^{A2}, R^{A3}, and R^{A4} that is not alkyl is hydrogen. In some embodiments is a compound of Formula (Ib) or a pharmaceutically acceptable salt thereof, wherein two of R^{A1}, R^{A2}, R^{A3}, and R^{A4} is alkyl, and each of R^{A1}, R^{A2}, R^{A3}, and R^{A4} that is not alkyl is hydrogen. In some embodiments is a compound of Formula (Ib) or a pharmaceutically acceptable salt thereof, wherein each of R^{A1}, R^{A2}, R^{A3}, and R^{A4} is hydrogen. In some embodiments is a compound of Formula (Ib) or a pharmaceutically acceptable salt thereof, wherein one of R^{A1}, R^{A2}, R^{A3}, and R^{A4} is alkyl, and each of R^{A1}, R^{A2}, R^{A3}, and R^{A4} that is not alkyl is hydrogen, wherein the alkyl is methyl, ethyl, isopropyl, or tert-butyl. In some embodiments is a compound of Formula (Ib) or a pharmaceutically acceptable salt thereof, wherein two of R^{A1}, R^{A2}, R^{A3}, and R^{A4} is alkyl, and each of R^{A1}, R^{A2}, R^{A3}, and R^{A4} that is not alkyl is hydrogen, wherein each alkyl is independently methyl, ethyl, isopropyl, or tert-butyl. In some embodiments is a compound of Formula (Ib) or a pharmaceutically acceptable salt thereof, wherein R^{A5} is heterocyclylalkyl. In some embodiments is a compound of Formula (Ib) or a pharmaceutically acceptable salt thereof, wherein R^{A5} is —OC(O)R¹⁵. In some embodiments is a compound of Formula (Ib) or a pharmaceutically acceptable salt thereof, wherein R^{A5} is —OC(O)R¹⁵, wherein R¹⁵ is alkyl. In some embodiments is a compound of Formula (Ib) or a pharmaceutically acceptable salt thereof, wherein R^{A5} is —OC(O)R¹⁵, wherein R¹⁵ is methyl, ethyl, isopropyl, n-propyl, tert-butyl, isobutyl, or n-butyl. In some embodiments is a compound of Formula (Ib) or a pharmaceutically acceptable salt thereof, wherein R^{A5} is —OC(O)R¹⁵, wherein R¹⁵ is isobutyl.

In some embodiments is a compound of Formula (I) having the structure of Formula (Ib-1), or a pharmaceutically acceptable salt thereof:

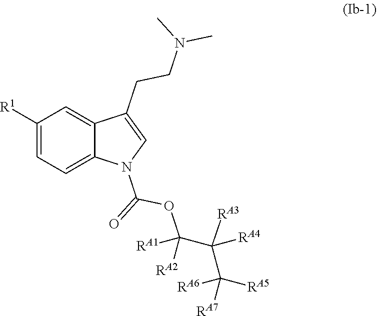

wherein:
R¹ is methoxy or hydrogen;
each of R^{A1}, R^{A2}, R^{A3}, R^{A4}, R^{A6}, and R^{A7} is independently hydrogen or alkyl that is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹, and
R^{A5} is heteroalkyl, heterocyclylalkyl, heteroaryl, or —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each heteroalkyl, heterocyclylalkyl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Ib-1) or a pharmaceutically acceptable salt thereof, wherein one of R^{A1}, R^{A2}, R^{A3}, R^{A4}, R^{A6}, and R^{A7} is alkyl, and each of R^{A1}, R^{A2}, R^{A3}, R^{A4}, R^{A6}, and R^{A7} that is not alkyl is hydrogen. In some embodiments is a compound of Formula (Ib-1) or a pharmaceutically acceptable salt thereof, wherein two of R^{A1}, R^{A2}, R^{A3}, R^{A4}, R^{A6}, and R^{A7} is alkyl, and each of R^{A1}, R^{A2}, R^{A3}, R^{A4}, R^{A6}, and R^{A7} that is not alkyl is hydrogen. In some embodiments is a compound of Formula (Ib-1) or a pharmaceutically acceptable salt thereof, wherein each of R^{A1}, R^{A2}, R^{A3}, R^{A4}, R^{A6}, and R^{A7} is hydrogen. In some embodiments is a compound of Formula (Ib-1) or a pharmaceutically acceptable salt thereof, wherein one of R^{A1}, R^{A2}, R^{A3}, R^{A4}, R^{A6}, and R^{A7} is alkyl, and each of R^{A1}, R^{A2}, R^{A3}, R^{A4}, R^{A6}, and R^{A7} that is not alkyl is hydrogen, wherein the alkyl is methyl, ethyl, isopropyl, or tert-butyl. In some embodiments is a compound of Formula (Ib-1) or a pharmaceutically acceptable salt thereof, wherein R^{A3} and R^{A4} are each independently alkyl, and each of R^{A1}, R^{A2}, R^{A6}, and R^{A7} is hydrogen In some embodiments is a compound of Formula (Ib-1) or a pharmaceutically acceptable salt thereof, wherein two of R^{A1}, R^{A2}, R^{A3}, R^{A4}, R^{A6}, and R^{A7} is alkyl, and each of R^{A1}, R^{A2}, R^{A3}, R^{A4}, R^{A6}, and R^{A7} that is not alkyl is hydrogen, wherein each alkyl is independently methyl, ethyl, isopropyl, or tert-butyl. In some embodiments is a compound of Formula (Ib-1) or a pharmaceutically acceptable salt thereof, wherein R^{A5} is heterocyclylalkyl. In some embodiments is a compound of Formula (Ib-1) or a pharmaceutically acceptable salt thereof, wherein R^{A5} is —OC(O)R¹⁵. In some embodiments is a compound of Formula (Ib-1) or a pharmaceutically acceptable salt thereof, wherein R^{A5} is —OC(O)R¹⁵, wherein R¹⁵ is alkyl. In some embodiments is a compound of Formula (Ib-1) or a pharmaceutically acceptable salt thereof, wherein R^{A5} is —OC(O)R¹⁵, wherein R¹⁵ is methyl, ethyl, isopropyl, n-propyl, tert-butyl, isobutyl, or n-butyl. In some embodiments is a compound of Formula (Ib-1) or a pharmaceutically acceptable salt thereof, wherein R^{A5} is —OC(O)R¹⁵, wherein R¹⁵ is isobutyl.

In some embodiments is a compound of Formula (I) or (Ib-1), or a pharmaceutically acceptable salt thereof, wherein the compound is:

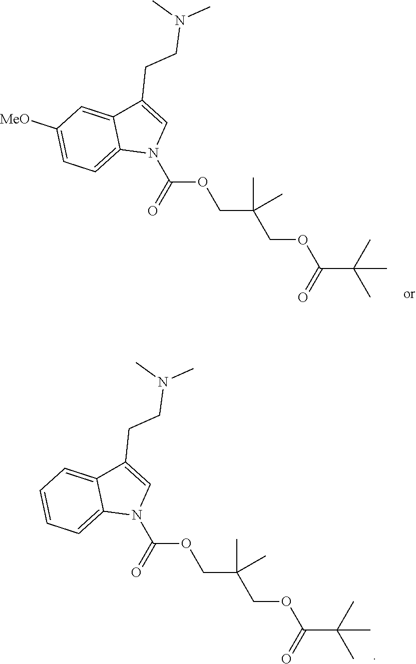 [SMILES: COc1ccc2c(c1)c(CCN(C)C)cn2C(=O)OCC(C)(C)COC(=O)C(C)(C)C ; CN(C)CCc1cn(C(=O)OCC(C)(C)COC(=O)C(C)(C)C)c2ccccc12]

In some embodiments is a compound of Formula (I) or (Ib) having the structure of Formula (Ib1), or a pharmaceutically acceptable salt thereof:

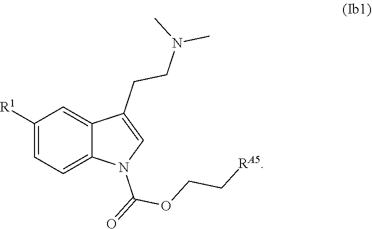

In some embodiments is a compound of Formula (Ib) or (Ib1), or a pharmaceutically acceptable salt thereof, wherein R^{A5} is heteroalkyl or heterocyclylalkyl. In some embodiments is a compound of Formula (Ib) or (Ib1), or a pharmaceutically acceptable salt thereof, wherein R^{A5} is heteroalkyl. In some embodiments is a compound of Formula (Ib) or (Ib1), or a pharmaceutically acceptable salt thereof, wherein R^{A5} is heteroalkyl that is unsubstituted. In some embodiments is a compound of Formula (Ib) or (Ib1), or a pharmaceutically acceptable salt thereof, wherein R^{A5} is heterocyclylalkyl. In some embodiments is a compound of Formula (Ib) or (Ib1), or a pharmaceutically acceptable salt thereof, wherein R^{A5} is heterocyclylalkyl that is unsubstituted. In some embodiments is a compound of Formula (Ib) or (Ib1), or a pharmaceutically acceptable salt thereof, wherein R^{A5} is methoxy, ethoxy, cyclopropyloxy, methylamino, or dimethylamino. In some embodiments is a compound of Formula (Ib) or (Ib1), or a pharmaceutically acceptable salt thereof, wherein R^{A5} is

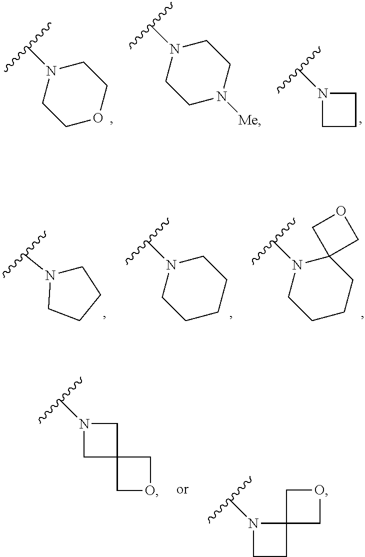

In some embodiments is a compound of Formula (Ib) or (Ib1), or a pharmaceutically acceptable salt thereof, wherein R^{A5} is —OC(O)R¹⁵.

In some embodiments is a compound of Formula (Ib) or (Ib1), or a pharmaceutically acceptable salt thereof, wherein R^{A5} is —OC(O)R¹⁵, wherein R¹⁵ is alkyl, cycloalkyl, aryl, or heteroaryl. In some embodiments is a compound of Formula (Ib) or (Ib1), or a pharmaceutically acceptable salt thereof, wherein R^{A5} is —OC(O)R¹⁵, wherein R¹⁵ is methyl, ethyl, n-propyl, isopropyl, n-butyl, tert-butyl, n-pentyl, or 3-methyl-1-butyl. In some embodiments is a compound of Formula (Ib) or (Ib1), or a pharmaceutically acceptable salt thereof, wherein R^{A5} is —OC(O)R¹⁵, wherein R¹⁵ is phenyl. In some embodiments is a compound of Formula (Ib) or (Ib1), or a pharmaceutically acceptable salt thereof, wherein R^{A5} is —OC(O)R¹⁵, wherein R¹⁵ is 2-pyridyl, 3-pyridyl, 4-pyridyl, 2-pyrimidyl, 4-pyrimidyl, 5-pyrimidyl, or 6-pyrimidyl.

In some embodiments is a compound of Formula (Ib) or (Ib1), or a pharmaceutically acceptable salt thereof, wherein R^{A5} is —N(R¹³)C(O)OR¹⁴. In some embodiments is a compound of Formula (Ib) or (Ib1), or a pharmaceutically acceptable salt thereof, wherein R^{A5} is —N(R¹³)C(O)OR¹⁴, wherein R¹³ is hydrogen or alkyl. In some embodiments is a compound of Formula (Ib) or (Ib1), or a pharmaceutically acceptable salt thereof, wherein R^{A5} is —N(R¹³)C(O)OR¹⁴, wherein R¹³ is hydrogen. In some embodiments is a compound of Formula (Ib) or (Ib1), or a pharmaceutically acceptable salt thereof, wherein R^{A5} is —N(R¹³)C(O)OR¹⁴, wherein R¹³ is alkyl. In some embodiments is a compound of Formula (Ib) or (Ib1), or a pharmaceutically acceptable salt thereof, wherein R^{A5} is —N(R¹³)C(O)OR¹⁴, wherein R¹³ is unsubstituted alkyl. In some embodiments is a compound of Formula (Ib) or (Ib1), or a pharmaceutically acceptable salt thereof, wherein R^{A5} is —N(R¹³)C(O)OR¹⁴, wherein R¹⁴ is methyl, ethyl, n-propyl, isopropyl, n-butyl, tert-butyl, n-pentyl, or 3-methyl-1-butyl.

In some embodiments is a compound of Formula (Ib) or (Ib1), or a pharmaceutically acceptable salt thereof, wherein R^{A5} is —N(R¹³)C(O)R¹⁴. In some embodiments is a compound of Formula (Ib) or (Ib1), or a pharmaceutically acceptable salt thereof, wherein R^{A5} is —N(R¹³)C(O)R¹⁴, wherein R¹³ is hydrogen or alkyl. In some embodiments is a compound of Formula (Ib) or (Ib1), or a pharmaceutically acceptable salt thereof, wherein R^{A5} is —N(R¹³)C(O)R¹⁴, wherein R¹³ is hydrogen. In some embodiments is a compound of Formula (Ib) or (Ib1), or a pharmaceutically acceptable salt thereof, wherein R^{A5} is —N(R¹³)C(O)R¹⁴, wherein R¹³ is alkyl. In some embodiments is a compound of Formula (Ib) or (Ib1), or a pharmaceutically acceptable salt thereof, wherein R^{A5} is —N(R¹³)C(O)R¹⁴, wherein R¹³ is unsubstituted alkyl. In some embodiments is a compound of Formula (Ib) or (Ib1), or a pharmaceutically acceptable salt thereof, wherein R^{A5} is —N(R¹³)C(O)R¹⁴, wherein R¹⁴ is methyl, ethyl, n-propyl, isopropyl, n-butyl, tert-butyl, n-pentyl, or 3-methyl-1-butyl. In some embodiments is a compound of Formula (Ib) or (Ib1), or a pharmaceutically acceptable salt thereof, wherein R^{A5} is —N(R¹³)C(O)R¹⁴, wherein R¹⁴ is phenyl. In some embodiments is a compound of Formula (Ib) or (Ib1), or a pharmaceutically acceptable salt thereof, wherein R^{A5} is —N(R¹³)C(O)R¹⁴, wherein R¹⁴ is 2-pyridyl, 3-pyridyl, 4-pyridyl, 2-pyrimidyl, 4-pyrimidyl, 5-pyrimidyl, or 6-pyrimidyl.

In some embodiments is a compound of Formula (I), (Ib), or (Ib1), or a pharmaceutically acceptable salt thereof, wherein the compound is:

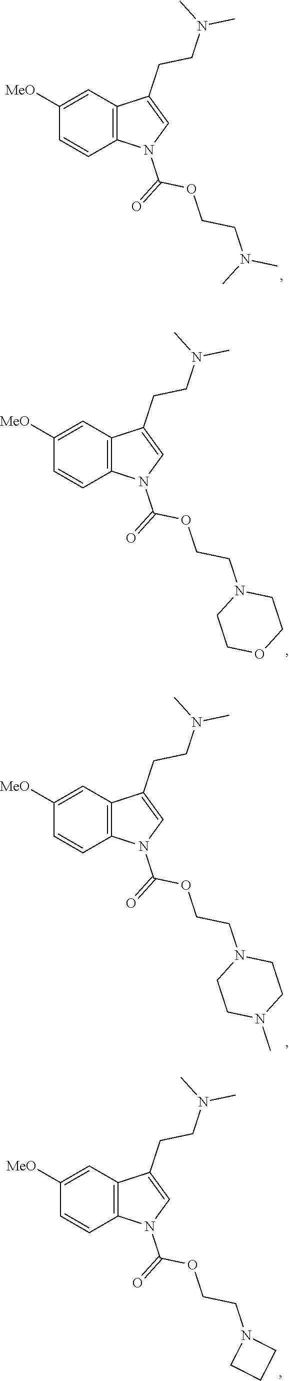 [SMILES: COc1ccc2c(c1)c(CCN(C)C)cn2C(=O)OCCN(C)C ; COc1ccc2c(c1)c(CCN(C)C)cn2C(=O)OCCN1CCOCC1 ; COc1ccc2c(c1)c(CCN(C)C)cn2C(=O)OCCN1CCN(C)CC1 ; COc1ccc2c(c1)c(CCN(C)C)cn2C(=O)OCCN1CCC1]

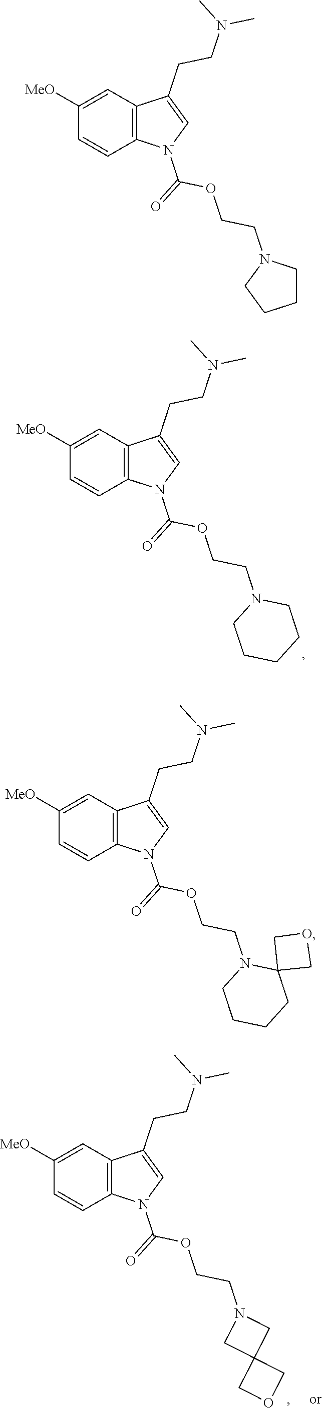 [SMILES: COc1ccc2c(c1)c(CCN(C)C)cn2C(=O)OCCN1CCCC1 ; COc1ccc2c(c1)c(CCN(C)C)cn2C(=O)OCCN1CCCCC1 ; COc1ccc2c(c1)c(CCN(C)C)cn2C(=O)OCCN1CCCCC12COC2 ; COc1ccc2c(c1)c(CCN(C)C)cn2C(=O)OCCN1CC2(COC2)C1]

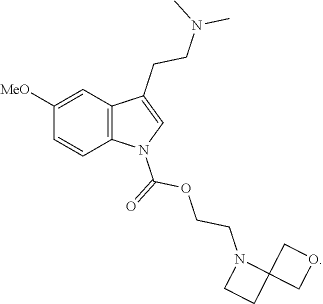 [SMILES: COc1ccc2c(c1)c(CCN(C)C)cn2C(=O)OCCN1CCC12COC2]

In some embodiments is a compound of Formula (I), (Ib), or (Ib1), or a pharmaceutically acceptable salt thereof, wherein the compound is:

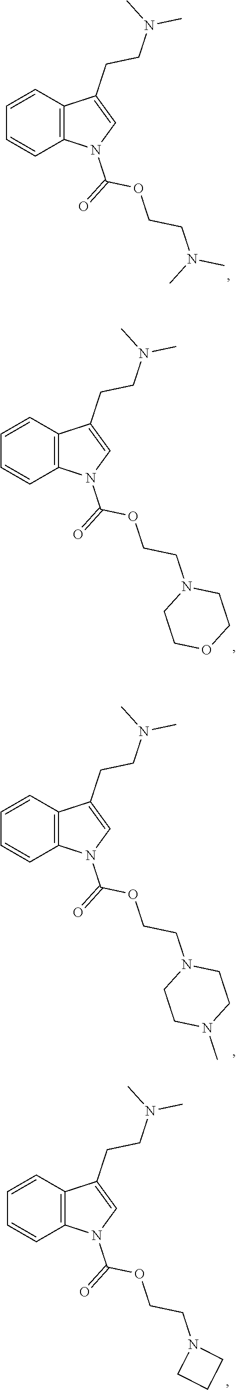 [SMILES: CN(C)CCOC(=O)n1cc(CCN(C)C)c2ccccc21 ; CN(C)CCc1cn(C(=O)OCCN2CCOCC2)c2ccccc12 ; CN(C)CCc1cn(C(=O)OCCN2CCN(C)CC2)c2ccccc12 ; CN(C)CCc1cn(C(=O)OCCN2CCC2)c2ccccc12]

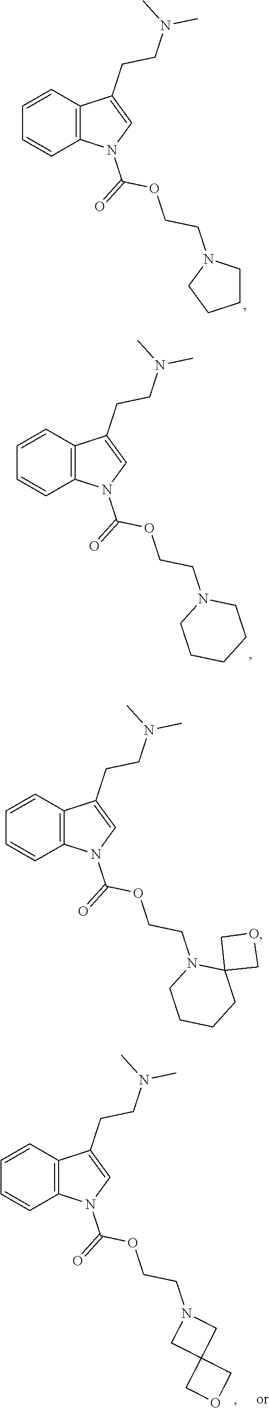 [SMILES: CN(C)CCc1cn(C(=O)OCCN2CCCC2)c2ccccc12 ; CN(C)CCc1cn(C(=O)OCCN2CCCCC2)c2ccccc12 ; CN(C)CCc1cn(C(=O)OCCN2CCCCC23COC3)c2ccccc12 ; CN(C)CCc1cn(C(=O)OCCN2CC3(COC3)C2)c2ccccc12]

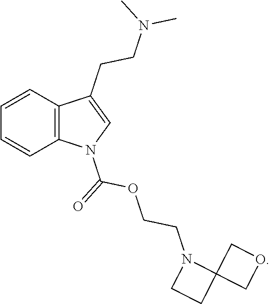 [SMILES: CN(C)CCc1cn(C(=O)OCCN2CCC23COC3)c2ccccc12]

In some embodiments is a compound of Formula (I), (Ib), or (Ib1), or a pharmaceutically acceptable salt thereof, wherein the compound is

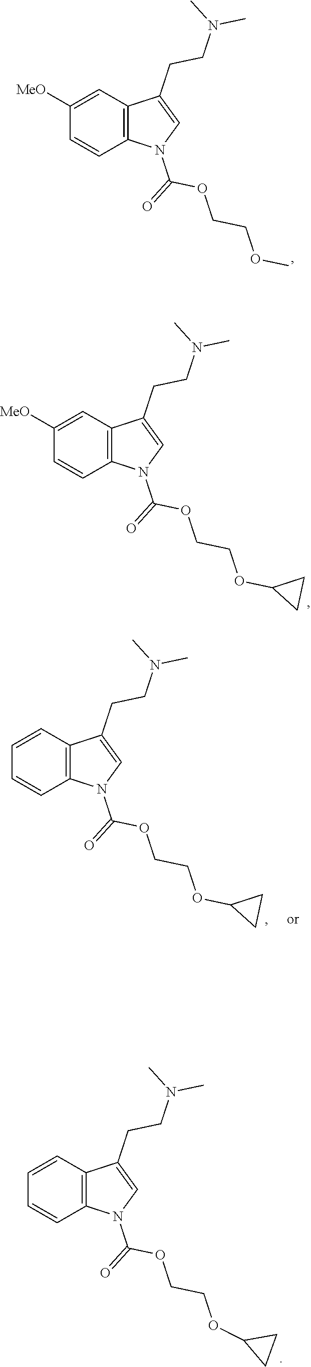 [SMILES: COCCOC(=O)n1cc(CCN(C)C)c2cc(OC)ccc21 ; COc1ccc2c(c1)c(CCN(C)C)cn2C(=O)OCCOC1CC1 ; CN(C)CCc1cn(C(=O)OCCOC2CC2)c2ccccc12 ; CN(C)CCc1cn(C(=O)OCCOC2CC2)c2ccccc12]

In some embodiments is a compound of Formula (I) having the structure of Formula (Ic), or a pharmaceutically acceptable salt thereof:

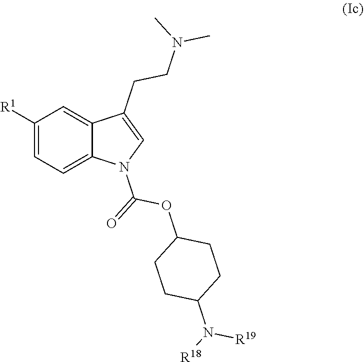

wherein R is hydrogen or methoxy, and each of R¹⁸ and R¹⁹ is independently hydrogen, alkyl, cycloalkyl, or heteroalkyl, wherein each alkyl, cycloalkyl, and heteroalkyl is independently unsubstituted or substituted with one or more R^{B}; or R¹⁸ and R¹⁹ together with the atom to which they are attached form a heterocyclylalkyl ring that is unsubstituted or substituted with one or more R^{B}.

In some embodiments is a compound of Formula (I) or (Ic), or a pharmaceutically acceptable salt or solvate thereof, wherein each of R¹⁸ and R¹⁹ is independently methyl, ethyl, n-propyl, isopropyl, cyclopropyl, tert-butyl, —CH₂CH₂OMe, or —CH₂CH₂SO₂Me. In some embodiments is a compound of Formula (I) or (Ic), or a pharmaceutically acceptable salt or solvate thereof, wherein R¹⁸ is hydrogen, and R¹⁹ is methyl, ethyl, n-propyl, isopropyl, cyclopropyl, tert-butyl, —CH₂CH₂OMe, or —CH₂CH₂SO₂Me. In some embodiments is a compound of Formula (I) or (Ic), or a pharmaceutically acceptable salt or solvate thereof, wherein each of R¹⁸ and R¹⁹ is methyl, ethyl, n-propyl, isopropyl, cyclopropyl, tert-butyl, —CH₂CH₂OMe, or —CH₂CH₂SO₂Me.

In some embodiments is a compound of Formula (I) or (Ic), or a pharmaceutically acceptable salt or solvate thereof, wherein R¹⁸ and R¹⁹ together with the atom to which they are attached form a heterocyclylalkyl ring that is substituted or unsubstituted. In some embodiments is a compound of Formula (I) or (Ic), or a pharmaceutically acceptable salt or solvate thereof, wherein R¹⁸ and R¹⁹ together with the atom to which they are attached form a azetidine ring, a pyrrolidine ring, or a piperidine ring, each of which is substituted or unsubstituted.

In some embodiments is a compound of Formula (I) or (Ic), or a pharmaceutically acceptable salt or solvate thereof, wherein the compound is:

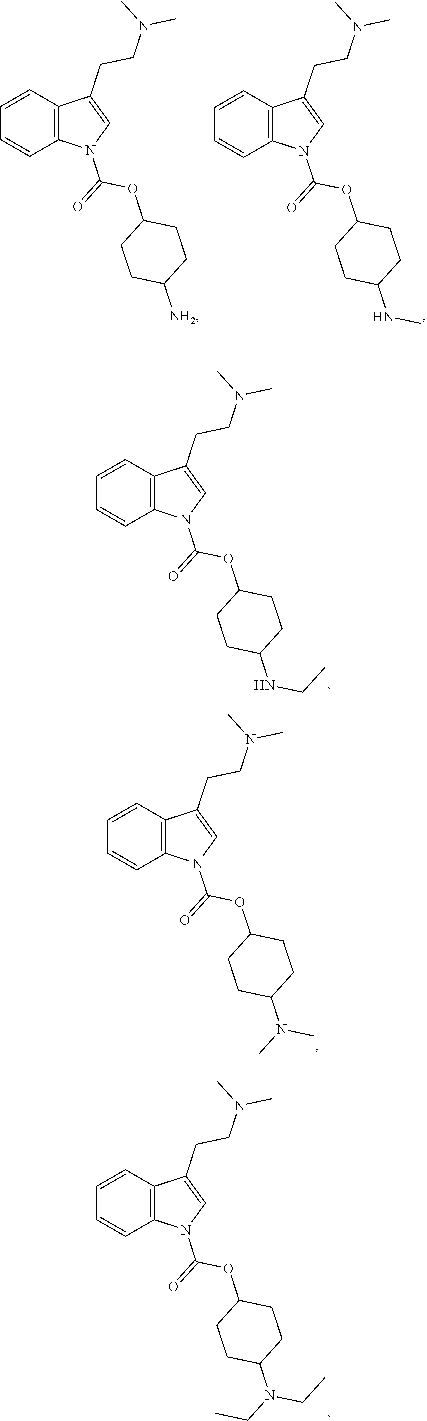

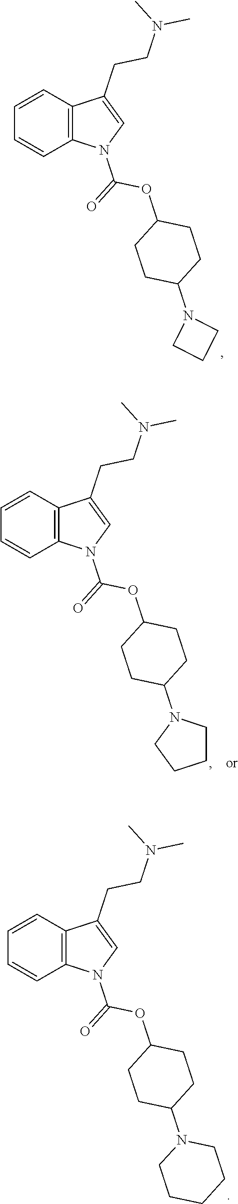 [SMILES: CN(C)CCc1cn(C(=O)OC2CCC(N3CCC3)CC2)c2ccccc12 ; CN(C)CCc1cn(C(=O)OC2CCC(N3CCCC3)CC2)c2ccccc12 ; CN(C)CCc1cn(C(=O)OC2CCC(N3CCCCC3)CC2)c2ccccc12]

In some embodiments is a compound of Formula (I) or (Ic), or a pharmaceutically acceptable salt or solvate thereof, wherein the compound is:

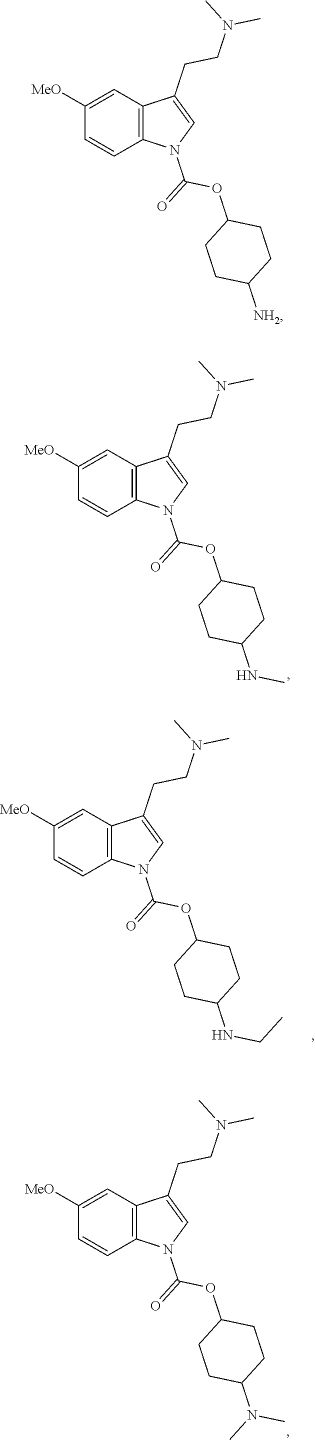 [SMILES: COc1ccc2c(c1)c(CCN(C)C)cn2C(=O)OC1CCC(N)CC1 ; CNC1CCC(OC(=O)n2cc(CCN(C)C)c3cc(OC)ccc32)CC1 ; CCNC1CCC(OC(=O)n2cc(CCN(C)C)c3cc(OC)ccc32)CC1 ; COc1ccc2c(c1)c(CCN(C)C)cn2C(=O)OC1CCC(N(C)C)CC1]

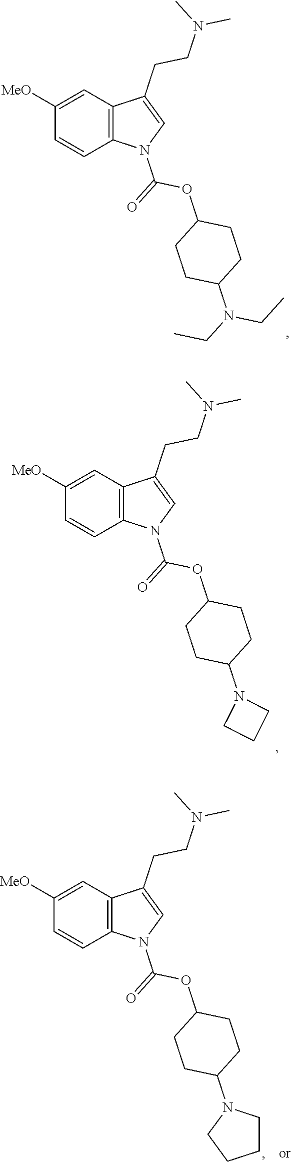

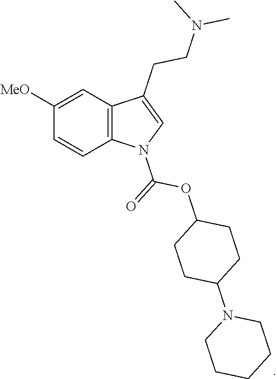 [SMILES: COc1ccc2c(c1)c(CCN(C)C)cn2C(=O)OC1CCC(N2CCCCC2)CC1]

In some embodiments is a compound of Formula (I) having the structure of Formula (Id), or a pharmaceutically acceptable salt thereof:

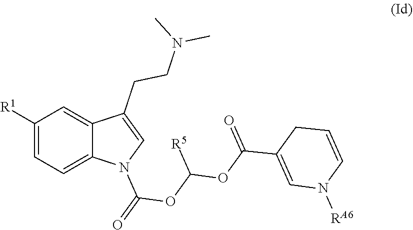

wherein: R¹ is hydrogen or methoxy; R⁵ is hydrogen, alkyl, or cycloalkyl, wherein each of alkyl and cycloalykl is independently unsubstituted or substituted with one or more R^{A}; and R^{A6} is hydrogen or alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Id), or a pharmaceutically acceptable salt or solvate thereof, wherein R⁵ is alkyl or cycloalkyl. In some embodiments is a compound of Formula (Id), or a pharmaceutically acceptable salt or solvate thereof, wherein R⁵ is unsubstituted alkyl. In some embodiments is a compound of Formula (Id), or a pharmaceutically acceptable salt or solvate thereof, wherein R⁵ is hydrogen, methyl, ethyl, or isopropyl. In some embodiments is a compound of Formula (Id), or a pharmaceutically acceptable salt or solvate thereof, wherein R^{A6} is alkyl. In some embodiments is a compound of Formula (Id), or a pharmaceutically acceptable salt or solvate thereof, wherein R^{A6} is hydrogen, methyl, ethyl, n-propyl, isopropyl, n-butyl, or benzyl. In some embodiments is a compound of Formula (Id), or a pharmaceutically acceptable salt or solvate thereof, wherein R⁵ is unsubstituted alkyl, and R^{A6} is hydrogen, methyl, ethyl, n-propyl, isopropyl, n-butyl, or benzyl. In some embodiments is a compound of Formula (Id), or a pharmaceutically acceptable salt or solvate thereof, wherein R⁵ is hydrogen, and R^{A6} is hydrogen, methyl, ethyl, n-propyl, isopropyl, n-butyl, or benzyl.

In some embodiments is a compound of Formula (Id), or a pharmaceutically

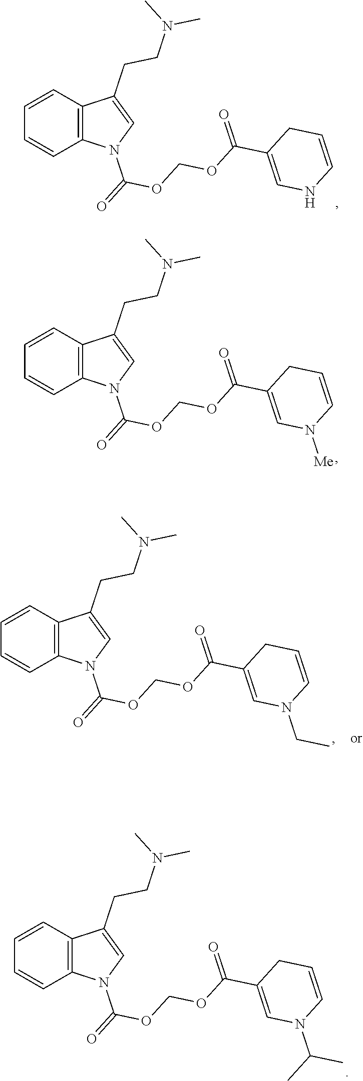 [SMILES: CN(C)CCc1cn(C(=O)OCOC(=O)C2=CNC=CC2)c2ccccc12 ; CN1C=CCC(C(=O)OCOC(=O)n2cc(CCN(C)C)c3ccccc32)=C1 ; CCN1C=CCC(C(=O)OCOC(=O)n2cc(CCN(C)C)c3ccccc32)=C1 ; CC(C)N1C=CCC(C(=O)OCOC(=O)n2cc(CCN(C)C)c3ccccc32)=C1]

In some embodiments is a compound of Formula (Id), or a pharmaceutically acceptable salt or solvate thereof, wherein the compound is:

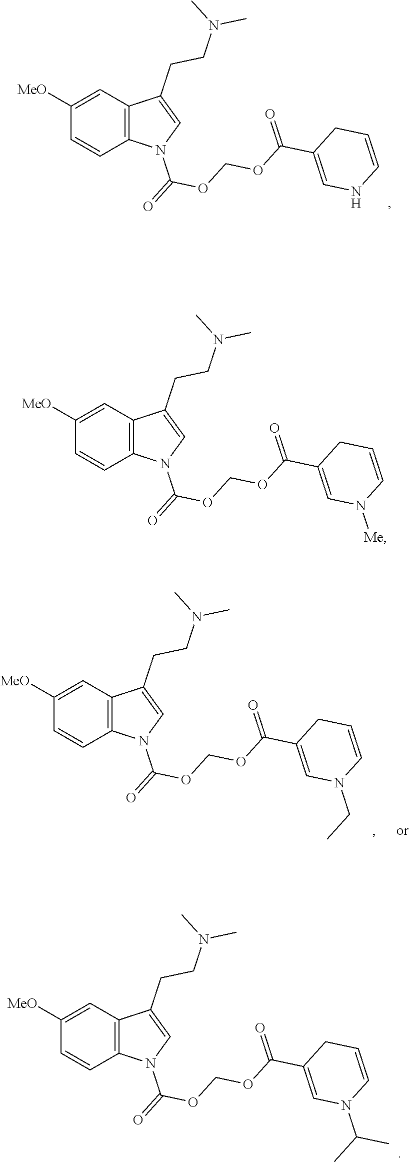 [SMILES: COc1ccc2c(c1)c(CCN(C)C)cn2C(=O)OCOC(=O)C1=CNC=CC1 ; COc1ccc2c(c1)c(CCN(C)C)cn2C(=O)OCOC(=O)C1=CN(C)C=CC1 ; CCN1C=CCC(C(=O)OCOC(=O)n2cc(CCN(C)C)c3cc(OC)ccc32)=C1 ; COc1ccc2c(c1)c(CCN(C)C)cn2C(=O)OCOC(=O)C1=CN(C(C)C)C=CC1]

In some embodiments is a compound of Formula (I) having the structure of Formula (Ie), or a pharmaceutically acceptable salt thereof:

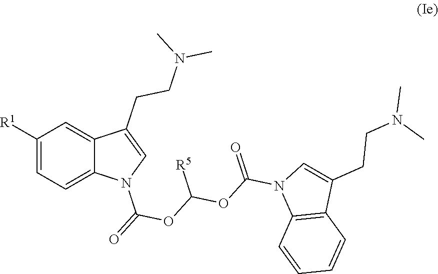

wherein R¹ is hydrogen or methoxy, and R⁵ is hydrogen, alkyl or cycloalkyl, wherein each alkyl and cycloalkyl is independently unsubstituted or substituted with one or more R^{A}.

In some embodiments is a compound of Formula (I) or (Ie), or a pharmaceutically acceptable salt or solvate thereof, wherein R⁵ is hydrogen. In some embodiments is a compound of Formula (I) or (Id), or a pharmaceutically acceptable salt or solvate thereof, wherein R⁵ is alkyl. In some embodiments is a compound of Formula (I) or (Ie), or a pharmaceutically acceptable salt or solvate thereof, wherein R⁵ is unsubstituted alkyl. In some embodiments is a compound of Formula (I) or (Ie), or a pharmaceutically acceptable salt or solvate thereof, wherein R⁵ is methyl, ethyl, or isopropyl.

In some embodiments is a compound of Formula (I) or (Ie), or a pharmaceutically acceptable salt or solvate thereof, wherein the compound is:

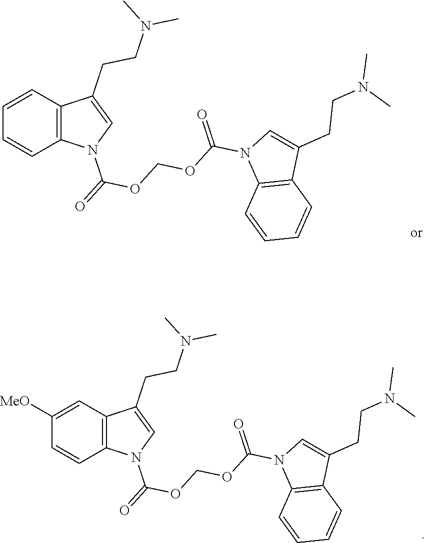 [SMILES: CN(C)CCc1cn(C(=O)OCOC(=O)n2cc(CCN(C)C)c3ccccc32)c2ccccc12 ; COc1ccc2c(c1)c(CCN(C)C)cn2C(=O)OCOC(=O)n1cc(CCN(C)C)c2ccccc21]

In some embodiments is a compound of Formula (I) having the structure of Formula (If), or a pharmaceutically acceptable salt thereof:

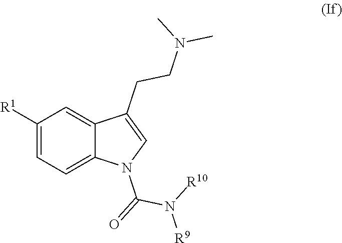

wherein:
R¹ is methoxy or hydrogen, and
each of R⁹ and R¹⁰ is independently hydrogen, alkyl, cycloalkyl, aryl, heteroaryl, heteroalkyl, or heterocyclylalkyl, wherein each alkyl, cycloalkyl, aryl, heteroaryl, heteroalkyl, and heterocyclylalkyl is independently unsubstituted or substituted with one or more R^{A}, or R⁹ and R¹⁰ together with the atom to which they are attached form a heterocyclylalkyl ring that is unsubstituted or substituted with one or more R^{A}.

In some embodiments is a compound of Formula (I) or (If), or a pharmaceutically acceptable salt or solvate thereof, wherein each of R⁹ and R¹⁰ is independently alkyl. In some embodiments is a compound of Formula (I) or (If), or a pharmaceutically acceptable salt or solvate thereof, wherein R¹⁰ is alkyl, cycloalkyl, aryl, heteroaryl, heteroalkyl, or heterocyclylalkyl. In some embodiments is a compound of Formula (I) or (If), or a pharmaceutically acceptable salt or solvate thereof, wherein each of R⁹ and R¹⁰ is independently unsubstituted alkyl. In some embodiments is a compound of Formula (I) or (If), or a pharmaceutically acceptable salt or solvate thereof, wherein R¹ is methoxy, and each of R⁹ and R¹⁰ is independently unsubstituted alkyl. In some embodiments is a compound of Formula (I) or (If), or a pharmaceutically acceptable salt or solvate thereof, wherein R¹ is hydrogen, and each of R⁹ and R¹⁰ is independently unsubstituted alkyl. In some embodiments is a compound of Formula (I) or (If), or a pharmaceutically acceptable salt or solvate thereof, wherein each of R⁹ and R¹⁰ is independently heteroalkyl. In some embodiments is a compound of Formula (I) or (If), or a pharmaceutically acceptable salt or solvate thereof, wherein each of R⁹ and R¹⁰ is independently unsubstituted heteroalkyl. In some embodiments is a compound of Formula (I) or (If), or a pharmaceutically acceptable salt or solvate thereof, wherein each of R⁹ and R¹⁰ is independently methyl, ethyl, n-propyl, isopropyl, n-butyl, cyclobutyl, cyclopentyl, cyclohexyl, cycloheptyl, cyclooctyl, tert-butyl, n-pentyl, n-heptyl, n-octyl, n-nonyl, or 3-methyl-1-butyl. In some embodiments is a compound of Formula (I) or (If), or a pharmaceutically acceptable salt or solvate thereof, wherein each of R⁹ and R¹⁰ is independently CH₂CHF₂, CH₂CF₃, or CH₂cPr. In some embodiments is a compound of Formula (I) or (If), or a pharmaceutically acceptable salt or solvate thereof, wherein each of R⁹ and R¹⁰ is phenyl. In some embodiments is a compound of Formula (I) or (If), or a pharmaceutically acceptable salt or solvate thereof, wherein each of R⁹ and R¹⁰ is independently 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (If), or a pharmaceutically acceptable salt or solvate thereof, wherein each of R⁹ and R¹⁰ is ethyl. In some embodiments is a compound of Formula (I) or (If), or a pharmaceutically acceptable salt or solvate thereof, wherein R¹ is hydrogen, and each of R⁹ and R¹⁰ is ethyl. In some embodiments is a compound of Formula (I) or (If), or a pharmaceutically acceptable salt or solvate thereof, wherein R¹ is methoxy, and each of R⁹ and R¹⁰ is ethyl. In some embodiments is a compound of Formula (I) or (If), or a pharmaceutically acceptable salt or solvate thereof, wherein each of R⁹ and R¹⁰ is methyl. In some embodiments is a compound of Formula (I) or (If), or a pharmaceutically acceptable salt or solvate thereof, wherein R¹ is hydrogen, and each of R⁹ and R¹⁰ is methyl. In some embodiments is a compound of Formula (I) or (If), or a pharmaceutically acceptable salt or solvate thereof, wherein R¹ is methoxy, and each of R⁹ and R¹⁰ is methyl.

In some embodiments is a compound of Formula (I) or (If), or a pharmaceutically acceptable salt or solvate thereof, wherein R¹ is methoxy or hydrogen, R⁹ is hydrogen, and R¹⁰ is alkyl, cycloalkyl, aryl, heteroaryl, heteroalkyl, or heterocyclylalkyl. In some embodiments is a compound of Formula (I) or (If), or a pharmaceutically acceptable salt or solvate thereof, wherein R⁹ is hydrogen, and R¹⁰ is alkyl. In some embodiments is a compound of Formula (I) or (If), or a pharmaceutically acceptable salt or solvate thereof, wherein R¹ is methoxy, R⁹ is hydrogen, and R¹⁰ is alkyl. In some embodiments is a compound of Formula (I) or (If), or a pharmaceutically acceptable salt or solvate thereof, wherein R¹ is hydrogen, R⁹ is hydrogen, and R¹⁰ is alkyl. In some embodiments is a compound of Formula (I) or (If), or a pharmaceutically acceptable salt or solvate thereof, wherein R⁹ is hydrogen, and R¹⁰ is heteroalkyl. In some embodiments is a compound of Formula (I) or (If), or a pharmaceutically acceptable salt or solvate thereof, wherein R⁹ is hydrogen, and R¹⁰ is unsubstituted alkyl. In some embodiments is a compound of Formula (I) or (If), or a pharmaceutically acceptable salt or solvate thereof, wherein R¹ is methoxy, R⁹ is hydrogen, and R¹⁰ is unsubstituted alkyl. In some embodiments is a compound of Formula (I) or (If), or a pharmaceutically acceptable salt or solvate thereof, wherein R¹ is hydrogen, R⁹ is hydrogen, and R¹⁰ is unsubstituted alkyl. In some embodiments is a compound of Formula (I) or (If), or a pharmaceutically acceptable salt or solvate thereof, wherein R⁹ is hydrogen, and R¹⁰ is unsubstituted heteroalkyl. In some embodiments is a compound of Formula (I) or (If), or a pharmaceutically acceptable salt or solvate thereof, wherein R⁹ is hydrogen, and R¹⁰ is methyl, ethyl, n-propyl, isopropyl, n-butyl, cyclobutyl, cyclopentyl, cyclohexyl, cycloheptyl, cyclooctyl, tert-butyl, n-pentyl, n-heptyl, n-octyl, n-nonyl, or 3-methyl-1-butyl. In some embodiments is a compound of Formula (I) or (If), or a pharmaceutically acceptable salt or solvate thereof, wherein R⁹ is hydrogen and R¹⁰ is —CH₂CHF₂, —CH₂CF₃, or —CH₂cPr. In some embodiments is a compound of Formula (I) or (If), or a pharmaceutically acceptable salt or solvate thereof, wherein R⁹ is hydrogen, and R¹⁰ is phenyl. In some embodiments is a compound of Formula (I) or (If), or a pharmaceutically acceptable salt or solvate thereof, wherein R⁹ is hydrogen, and R¹⁰ is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (If), or a pharmaceutically acceptable salt or solvate thereof, wherein R⁹ is hydrogen, and R¹⁰ is ethyl. In some embodiments is a compound of Formula (I) or (If), or a pharmaceutically acceptable salt or solvate thereof, wherein R¹ is hydrogen, R⁹ is hydrogen, and R¹⁰ is ethyl. In some embodiments is a compound of Formula (I) or (If), or a pharmaceutically acceptable salt or solvate thereof, wherein R¹ is methoxy, R⁹ is hydrogen, and R¹⁰ is ethyl. In some embodiments is a compound of Formula (I) or (If), or a pharmaceutically acceptable salt or solvate thereof, wherein R¹⁰ is

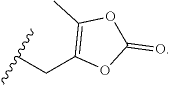 [SMILES: *Cc1oc(=O)oc1C]

In some embodiments is a compound of Formula (I) or (If), or a pharmaceutically acceptable salt or solvate thereof, wherein R¹ is methoxy, R⁹ is hydrogen, and R¹⁰ is

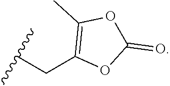 [SMILES: *Cc1oc(=O)oc1C]

In some embodiments is a compound of Formula (I) or (If), or a pharmaceutically acceptable salt or solvate thereof, wherein R¹ is hydrogen, R⁹ is hydrogen, and R¹⁰ is

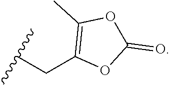 [SMILES: *Cc1oc(=O)oc1C]

In some embodiments is a compound of Formula (I) or (If), or a pharmaceutically acceptable salt thereof, wherein the compound is:

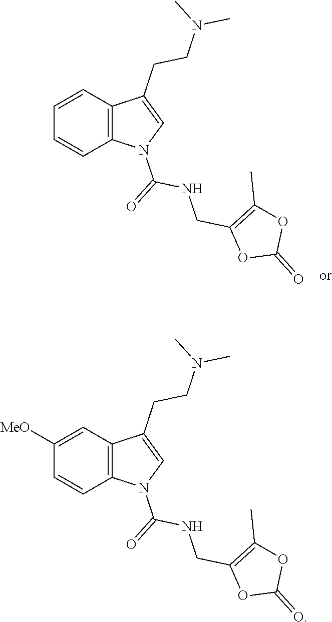 [SMILES: Cc1oc(=O)oc1CNC(=O)n1cc(CCN(C)C)c2ccccc21 ; COc1ccc2c(c1)c(CCN(C)C)cn2C(=O)NCc1oc(=O)oc1C]

In some embodiments is a compound of Formula (I) or (If) having the structure of Formula (If1), or a pharmaceutically acceptable salt thereof:

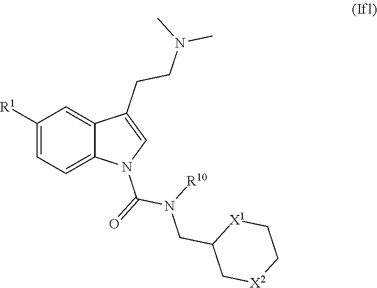

wherein:
R¹ is methoxy or hydrogen;
R¹⁰ is hydrogen, alkyl, heteroalkyl, cycloalkyl, or heterocyclylalkyl, wherein each of alkyl, heteroalkyl, cycloalkyl, and heterocyclylalkyl is unsubstituted or substituted with one or more R^{A}; and
each of X¹ and X² are independently selected from —CH₂—, —O—, —NH—, —S—, —S(O)—, —S(O)₂—, or —N(Y¹)—, wherein each Y¹ is independently hydrogen, cycloalkyl, heteroalkyl, or alkyl.

In some embodiments is a compound of Formula (If1), or a pharmaceutically acceptable salt or solvate thereof, wherein each Y¹ is independently hydrogen, methyl, ethyl, n-propyl, isopropyl, cyclopropyl, or CH₂CH₂OMe. In some embodiments is a compound of Formula (If1), or a pharmaceutically acceptable salt or solvate thereof, wherein X¹ is —CH₂— and X² is —N(Y¹)—. In some embodiments is a compound of Formula (If1), or a pharmaceutically acceptable salt or solvate thereof, wherein X² is —CH₂— and X¹ is —N(Y′)—. In some embodiments is a compound of Formula (If1), or a pharmaceutically acceptable salt or solvate thereof, wherein X¹ is —CH₂— and X² is —N(Y′)—, wherein Y¹ is hydrogen, methyl, ethyl, n-propyl, isopropyl, cyclopropyl, or —CH₂CH₂OMe. In some embodiments is a compound of Formula (If1), or a pharmaceutically acceptable salt or solvate thereof, wherein X² is —CH₂— and X¹ is —N(Y¹)—, wherein Y¹ is hydrogen, methyl, ethyl, n-propyl, isopropyl, cyclopropyl, or —CH₂CH₂OMe. In some embodiments is a compound of Formula (If1), or a pharmaceutically acceptable salt or solvate thereof, wherein each of X¹ and X² are —O— or —NH—. In some embodiments is a compound of Formula (If1), or a pharmaceutically acceptable salt or solvate thereof, wherein R¹⁰ is hydrogen.

In some embodiments is a compound of Formula (I) having the structure of Formula (Ig), or a pharmaceutically acceptable salt thereof:

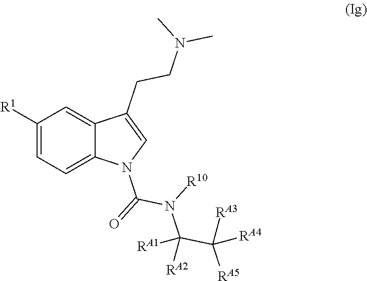

wherein:
R¹ is methoxy or hydrogen;
each of R^{A1}, R^{A2}, R^{A3}, and R^{A4} is independently hydrogen or alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R¹⁰ is hydrogen, alkyl, heteroalkyl, or cycloalkyl, wherein each of alkyl, heteroalkyl, and cycloalkyl is unsubstituted or substituted with one or more R^{A}; and
R^{A5} is heteroalkyl, heterocyclylalkyl, heteroaryl, or —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each of heteroalkyl, heterocyclylalkyl, heteroaryl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Ig), or a pharmaceutically acceptable salt or solvate thereof, wherein one R^{A1}, R^{A2}, R^{A3}, and R^{A4} is alkyl, and each of R^{A1}, R^{A2}, R^{A3}, and R^{A4} that is not alkyl is hydrogen. In some embodiments is a compound of Formula (Ig), or a pharmaceutically acceptable salt or solvate thereof, wherein two of R^{A1}, R^{A2}, R^{A3}, and R^{A4} is alkyl, and each of R^{A1}, R^{A2}, R^{A3}, and R^{A4} that is not alkyl is hydrogen. In some embodiments is a compound of Formula (Ig), or a pharmaceutically acceptable salt or solvate thereof, wherein each of R^{A1}, R^{A2}, R^{A3}, and R^{A4} is hydrogen. In some embodiments is a compound of Formula (Ig) or a pharmaceutically acceptable salt thereof, wherein one R^{A1}, R^{A2}, R^{A3}, and R^{A4} is alkyl, and each of R^{A1}, R^{A2}, R^{A3}, and R^{A4} that is not alkyl is hydrogen, wherein each alkyl is independently methyl, ethyl, tert-butyl, or isopropyl. In some embodiments is a compound of Formula (Ig), or a pharmaceutically acceptable salt or solvate thereof, wherein two of R^{A1}, R^{A2}, R^{A3}, and R^{A4} is alkyl, and each of R^{A1}, R^{A2}, R^{A3}, and R^{A4} that is not alkyl is hydrogen, wherein each alkyl is independently methyl, ethyl, tert-butyl, or isopropyl.

In some embodiments is a compound of Formula (Ig), or a pharmaceutically acceptable salt or solvate thereof, wherein R^{A5} is heteroalkyl. In some embodiments is a compound of Formula (Ig), or a pharmaceutically acceptable salt or solvate thereof, wherein R^{A5} is heteroalkyl that is unsubstituted. In some embodiments is a compound of Formula (Ig), or a pharmaceutically acceptable salt or solvate thereof, wherein R^{A5} is heterocyclylalkyl. In some embodiments is a compound of Formula (Ig), or a pharmaceutically acceptable salt or solvate thereof, wherein R^{A5} is heterocyclylalkyl that is unsubstituted. In some embodiments is a compound of Formula (Ig), or a pharmaceutically acceptable salt or solvate thereof, wherein R^{A5} is methoxy, ethoxy, cyclopropyloxy, methylamino, or dimethylamino. In some embodiments is a compound of Formula (Ig), or a pharmaceutically acceptable salt or solvate thereof, wherein R^{A5} is

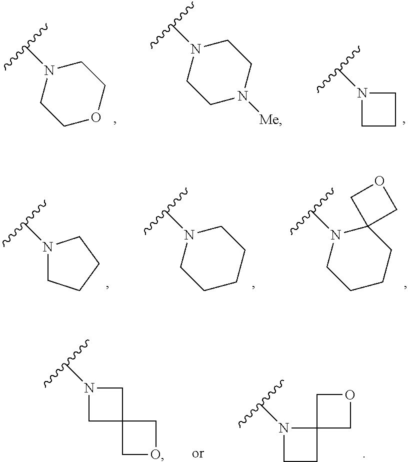

In some embodiments is a compound of Formula (Ig), or a pharmaceutically acceptable salt or solvate thereof, wherein R¹⁰ is hydrogen. In some embodiments is a compound of Formula (Ig), or a pharmaceutically acceptable salt or solvate thereof, wherein R¹⁰ is hydrogen, methyl, ethyl, n-propyl, or isopropyl. In some embodiments is a compound of Formula (Ig), or a pharmaceutically acceptable salt or solvate thereof, wherein R¹⁰ is —CH₂CH₂OMe or —CH₂CH₂SO₂Me.

In some embodiments is a compound of Formula (Ig), or a pharmaceutically acceptable salt or solvate thereof, wherein R^{A5} is —OC(O)R¹⁵. In some embodiments is a compound of Formula (Ig), or a pharmaceutically acceptable salt or solvate thereof, wherein R^{A5} is —OC(O)R¹⁵, wherein R¹⁵ is alkyl, cycloalkyl, aryl, or heteroaryl. In some embodiments is a compound of Formula (Ig), or a pharmaceutically acceptable salt or solvate thereof, wherein R^{A5} is —OC(O)R¹⁵, wherein R¹⁵ is methyl, ethyl, n-propyl, isopropyl, n-butyl, tert-butyl, n-pentyl, or 3-methyl-1-butyl. In some embodiments is a compound of Formula (Ig), or a pharmaceutically acceptable salt or solvate thereof, wherein R^{A5} is —OC(O)R¹⁵, wherein R¹⁵ is phenyl. In some embodiments is a compound of Formula (Ig), or a pharmaceutically acceptable salt or solvate thereof, wherein R^{A5} is —OC(O)R¹⁵, wherein R¹⁵ is 2-pyridyl, 3-pyridyl, 4-pyridyl, 2-pyrimidyl, 4-pyrimidyl, 5-pyrimidyl, or 6-pyrimidyl.

In some embodiments is a compound of Formula (Ig), or a pharmaceutically acceptable salt or solvate thereof, wherein R^{A5} is N(R¹³)C(O)OR¹⁴. In some embodiments is a compound of Formula (Ig), or a pharmaceutically acceptable salt or solvate thereof, wherein R^{A5} is N(R¹³)C(O)OR¹⁴, wherein R¹³ is hydrogen or alkyl. In some embodiments is a compound of Formula (Ig), or a pharmaceutically acceptable salt or solvate thereof, wherein R^{A5} is N(R¹³)C(O)OR¹⁴, wherein R¹³ is hydrogen. In some embodiments is a compound of Formula (Ig), or a pharmaceutically acceptable salt or solvate thereof, wherein R^{A5} is N(R¹³)C(O)OR¹⁴, wherein R¹³ is alkyl. In some embodiments is a compound of Formula (Ig), or a pharmaceutically acceptable salt or solvate thereof, wherein R^{A5} is —N(R¹³)C(O)OR¹⁴, wherein R¹³ is unsubstituted alkyl. In some embodiments is a compound of Formula (Ig), or a pharmaceutically acceptable salt or solvate thereof, wherein R^{A5} is —N(R¹³)C(O)OR¹⁴, wherein R¹⁴ is methyl, ethyl, n-propyl, isopropyl, n-butyl, tert-butyl, n-pentyl, or 3-methyl-1-butyl.

In some embodiments is a compound of Formula (Ig), or a pharmaceutically acceptable salt or solvate thereof, wherein R^{A5} is —N(R¹³)C(O)R¹⁴. In some embodiments is a compound of Formula (Ig), or a pharmaceutically acceptable salt or solvate thereof, wherein R^{A5} is —N(R¹³)C(O)R¹⁴, wherein R¹³ is hydrogen or alkyl. In some embodiments is a compound of Formula (Ig), or a pharmaceutically acceptable salt or solvate thereof, wherein R^{A5} is —N(R¹³)C(O)R¹⁴, wherein R¹³ is hydrogen. In some embodiments is a compound of Formula (Ig), or a pharmaceutically acceptable salt or solvate thereof, wherein R^{A5} is —N(R¹³)C(O)R¹⁴, wherein R¹³ is alkyl. In some embodiments is a compound of Formula (Ig), or a pharmaceutically acceptable salt or solvate thereof, wherein R^{A5} is —N(R¹³)C(O)R¹⁴, wherein R¹³ is unsubstituted alkyl. In some embodiments is a compound of Formula (Ig), or a pharmaceutically acceptable salt or solvate thereof, wherein R^{A5} is —N(R¹³)C(O)R¹⁴, wherein R¹⁴ is alkyl. In some embodiments is a compound of Formula (Ig), or a pharmaceutically acceptable salt or solvate thereof, wherein R^{A5} is —N(R¹³)C(O)R¹⁴, wherein R¹⁴ is unsubstituted alkyl. In some embodiments is a compound of Formula (Ig), or a pharmaceutically acceptable salt or solvate thereof, wherein R^{A5} is —N(R¹³)C(O)R¹⁴, wherein R¹⁴ is methyl, ethyl, n-propyl, isopropyl, n-butyl, tert-butyl, n-pentyl, or 3-methyl-1-butyl. In some embodiments is a compound of Formula (Ig), or a pharmaceutically acceptable salt or solvate thereof, wherein R^{A5} is —N(R¹³)C(O)R¹⁴, wherein R¹⁴ is phenyl. In some embodiments is a compound of Formula (Ig), or a pharmaceutically acceptable salt or solvate thereof, wherein R^{A5} is —N(R¹³)C(O)R¹⁴, wherein R¹⁴ is 2-pyridyl, 3-pyridyl, 4-pyridyl, 2-pyrimidyl, 4-pyrimidyl, 5-pyrimidyl, or 6-pyrimidyl.

In some embodiments is a compound of Formula (I) or (Ig), or a pharmaceutically acceptable salt thereof, wherein the compound is:

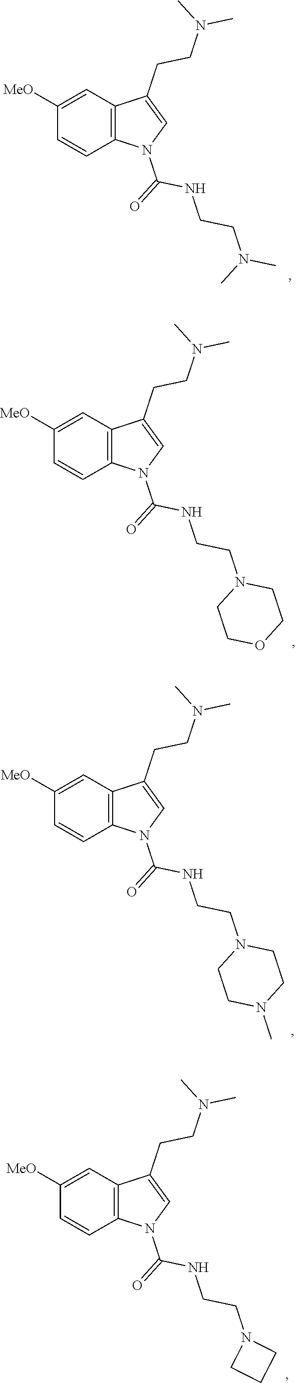 [SMILES: COc1ccc2c(c1)c(CCN(C)C)cn2C(=O)NCCN(C)C ; COc1ccc2c(c1)c(CCN(C)C)cn2C(=O)NCCN1CCOCC1 ; COc1ccc2c(c1)c(CCN(C)C)cn2C(=O)NCCN1CCN(C)CC1 ; COc1ccc2c(c1)c(CCN(C)C)cn2C(=O)NCCN1CCC1]

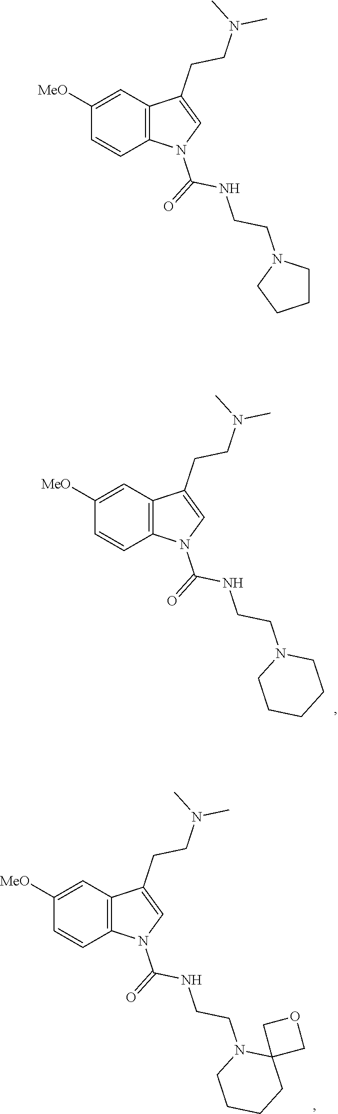 [SMILES: COc1ccc2c(c1)c(CCN(C)C)cn2C(=O)NCCN1CCCC1 ; COc1ccc2c(c1)c(CCN(C)C)cn2C(=O)NCCN1CCCCC1 ; COc1ccc2c(c1)c(CCN(C)C)cn2C(=O)NCCN1CCCCC12COC2]

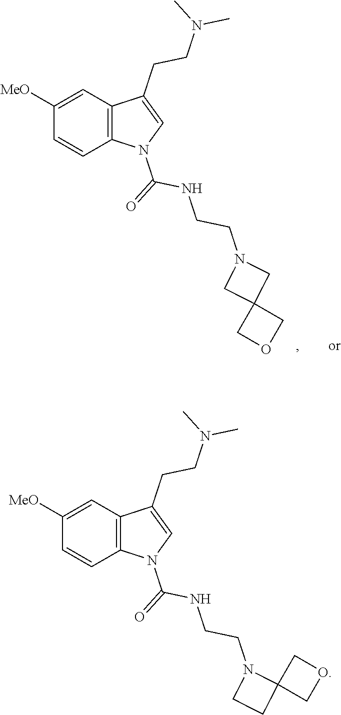 [SMILES: COc1ccc2c(c1)c(CCN(C)C)cn2C(=O)NCCN1CC2(COC2)C1 ; COc1ccc2c(c1)c(CCN(C)C)cn2C(=O)NCCN1CCC12COC2]

In some embodiments is a compound of Formula (I) or (Ig), or a pharmaceutically acceptable salt thereof, wherein the compound is:

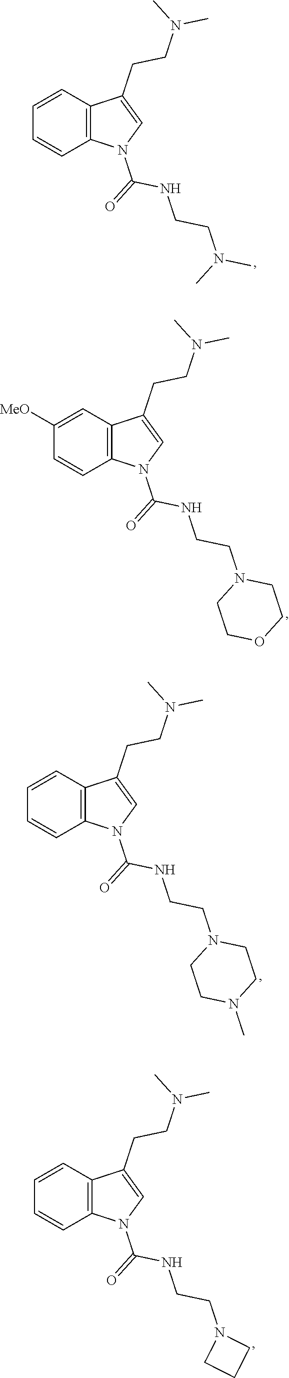

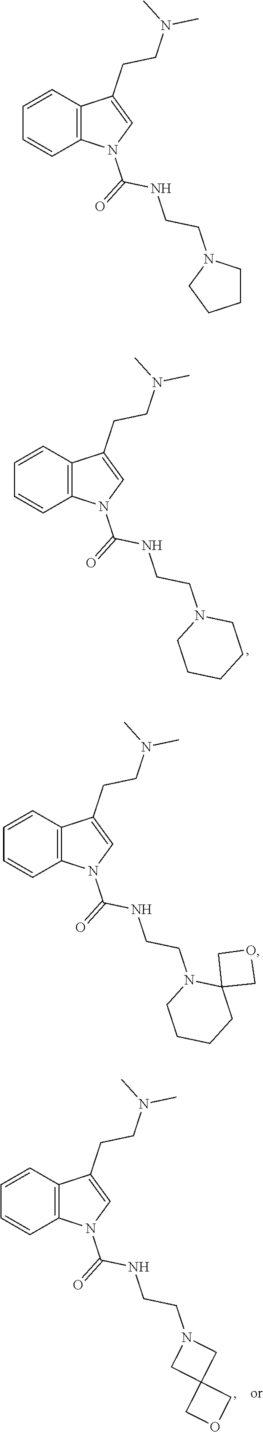 [SMILES: CN(C)CCc1cn(C(=O)NCCN2CCCC2)c2ccccc12 ; CN(C)CCc1cn(C(=O)NCCN2CCCCC2)c2ccccc12 ; CN(C)CCc1cn(C(=O)NCCN2CCCCC23COC3)c2ccccc12 ; CN(C)CCc1cn(C(=O)NCCN2CC3(COC3)C2)c2ccccc12]

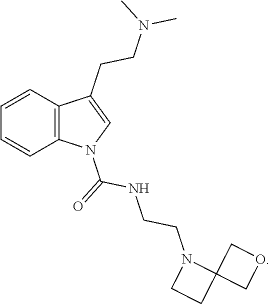 [SMILES: CN(C)CCc1cn(C(=O)NCCN2CCC23COC3)c2ccccc12]

In some embodiments is a compound of Formula (I) or (Ig), or a pharmaceutically acceptable salt thereof, wherein the compound is:

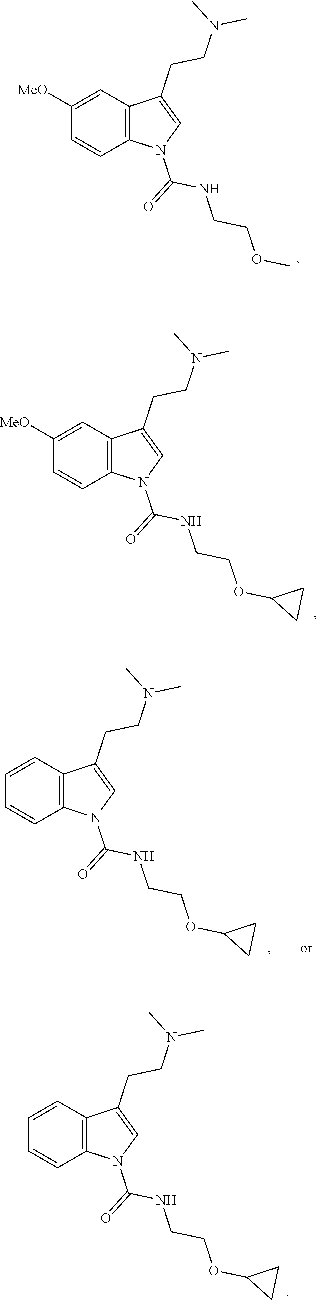 [SMILES: COCCNC(=O)n1cc(CCN(C)C)c2cc(OC)ccc21 ; COc1ccc2c(c1)c(CCN(C)C)cn2C(=O)NCCOC1CC1 ; CN(C)CCc1cn(C(=O)NCCOC2CC2)c2ccccc12 ; CN(C)CCc1cn(C(=O)NCCOC2CC2)c2ccccc12]

In some embodiments is a compound of Formula (I) having the structure of Formula (Ih), or a pharmaceutically acceptable salt thereof:

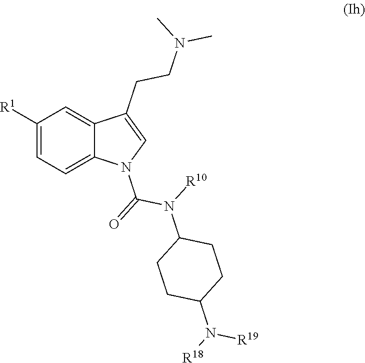

wherein:
R¹ is hydrogen or methoxy;
R¹⁰ is hydrogen, alkyl, heteroalkyl, or cycloalkyl, wherein each of alkyl, heteroalkyl, and cycloalkyl is unsubstituted or substituted with one or more R^{A}; and
each of R¹⁸ and R¹⁹ is independently hydrogen, alkyl, cycloalkyl, or heteroalkyl, wherein each alkyl, cycloalkyl, or heterocyclylalkyl is independently unsubstituted or substituted with one or more R^{B}; or R¹⁸ and R¹⁹ together with the atom to which they are attached form a heterocyclylalkyl ring that is unsubstituted or substituted with one or more R^{B}.

In some embodiments is a compound of Formula (Ih), or a pharmaceutically acceptable salt or solvate thereof, wherein R¹⁰ is hydrogen. In some embodiments is a compound of Formula (Ih), or a pharmaceutically acceptable salt or solvate thereof, wherein R¹⁰ is hydrogen, methyl, ethyl, n-propyl, or isopropyl. In some embodiments is a compound of Formula (Ih), or a pharmaceutically acceptable salt or solvate thereof, wherein R¹⁰ is CH₂CH₂OMe or CH₂CH₂SO₂Me.

In some embodiments is a compound of Formula (Ih), or a pharmaceutically acceptable salt or solvate thereof, wherein each of R¹⁸ and R¹⁹ is independently methyl, ethyl, n-propyl, isopropyl, cyclopropyl, tert-butyl, CH₂CH₂OMe, or CH₂CH₂SO₂Me. In some embodiments is a compound of Formula (Ih), or a pharmaceutically acceptable salt or solvate thereof, wherein R¹⁸ is hydrogen, and R¹⁹ is methyl, ethyl, n-propyl, isopropyl, cyclopropyl, tert-butyl, CH₂CH₂OMe, or CH₂CH₂SO₂Me. In some embodiments is a compound of Formula (Ih), or a pharmaceutically acceptable salt or solvate thereof, wherein each of R¹⁸ and R¹⁹ is methyl, ethyl, n-propyl, isopropyl, cyclopropyl, tert-butyl, CH₂CH₂OMe, or CH₂CH₂SO₂Me.

In some embodiments is a compound of Formula (Ih), or a pharmaceutically acceptable salt or solvate thereof, wherein R¹⁸ and R¹⁹ together with the atom to which they are attached form a heterocyclylalkyl ring. In some embodiments is a compound of Formula (Ih), or a pharmaceutically acceptable salt or solvate thereof, wherein R¹⁸ and R¹⁹ together with the atom to which they are attached form a azetidine ring, a morpholine ring, a pyrrolidine ring, or a piperidine ring, each of which is substituted or unsubstituted. In some embodiments is a compound of Formula (Ih), or a pharmaceutically acceptable salt or solvate thereof, wherein R¹⁸ and R¹⁹ together with the atom to which they are attached form a azetidine ring, a morpholine ring, a pyrrolidine ring, or a piperidine ring.

In some embodiments is a compound of Formula (I) or (Ih), or a pharmaceutically acceptable salt or solvate thereof, wherein the compound is:

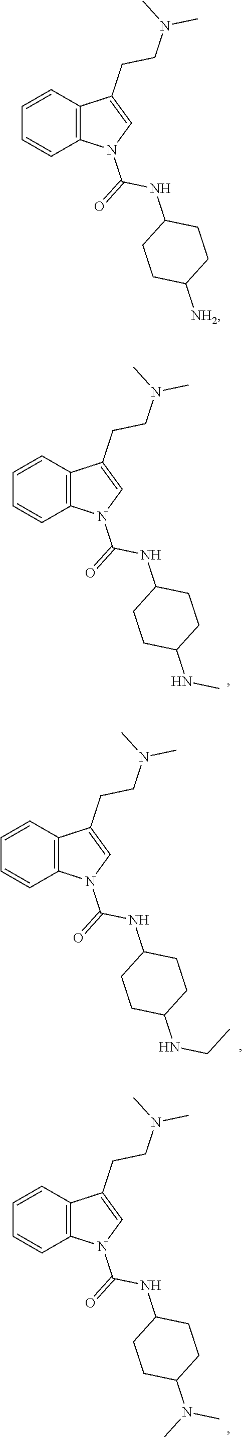 [SMILES: CN(C)CCc1cn(C(=O)NC2CCC(N)CC2)c2ccccc12 ; CNC1CCC(NC(=O)n2cc(CCN(C)C)c3ccccc32)CC1 ; CCNC1CCC(NC(=O)n2cc(CCN(C)C)c3ccccc32)CC1 ; CN(C)CCc1cn(C(=O)NC2CCC(N(C)C)CC2)c2ccccc12]

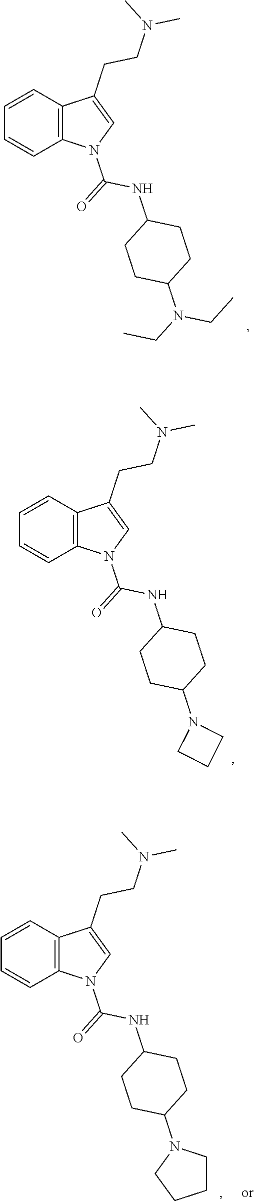 [SMILES: CCN(CC)C1CCC(NC(=O)n2cc(CCN(C)C)c3ccccc32)CC1 ; CN(C)CCc1cn(C(=O)NC2CCC(N3CCC3)CC2)c2ccccc12 ; CN(C)CCc1cn(C(=O)NC2CCC(N3CCCC3)CC2)c2ccccc12]

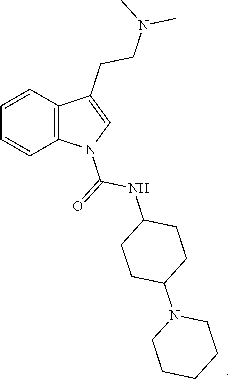 [SMILES: CN(C)CCc1cn(C(=O)NC2CCC(N3CCCCC3)CC2)c2ccccc12]

In some embodiments is a compound of Formula (I) or (Ih), or a pharmaceutically acceptable salt or solvate thereof, wherein the compound is:

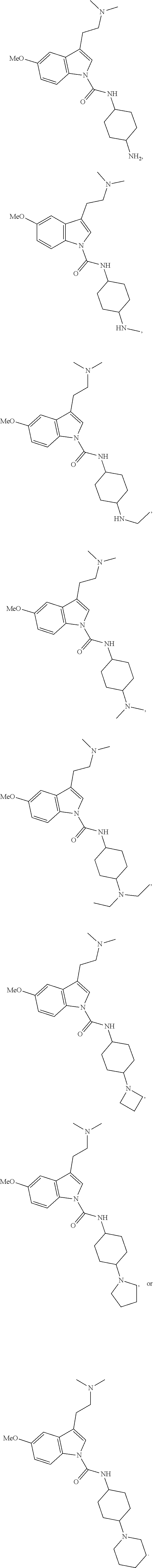

In some embodiments is a compound of Formula (I) having the structure of Formula (Ii), or a pharmaceutically acceptable salt thereof:

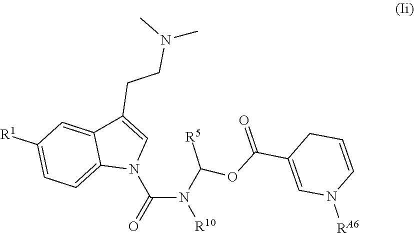

wherein:
R¹ is hydrogen or methoxy; and
each of R⁵ and R¹⁰ is independently hydrogen, alkyl, heteroalkyl, or cycloalkyl, wherein each alkyl, heteroalkyl, and cycloalkyl is independently unsubstituted or substituted with one or more R^{A}; and
R^{A6} is independently hydrogen, alkyl, heteroalkyl, or cycloalkyl, wherein each of alkyl, heteroalkyl, or cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Ii), or a pharmaceutically acceptable salt or solvate thereof, wherein R⁵ is alkyl. In some embodiments is a compound of Formula (Ii), or a pharmaceutically acceptable salt or solvate thereof, wherein R⁵ is unsubstituted alkyl. In some embodiments is a compound of Formula (Ii), or a pharmaceutically acceptable salt or solvate thereof, wherein R⁵ is methyl, ethyl, or isopropyl. In some embodiments is a compound of Formula (Ii), or a pharmaceutically acceptable salt or solvate thereof, wherein R⁵ is hydrogen, methyl, ethyl, or isopropyl. In some embodiments is a compound of Formula (Ii), or a pharmaceutically acceptable salt or solvate thereof, wherein R^{A6} is hydrogen. In some embodiments is a compound of Formula (Ii), or a pharmaceutically acceptable salt or solvate thereof, wherein R^{A6} is alkyl. In some embodiments is a compound of Formula (Ii), or a pharmaceutically acceptable salt or solvate thereof, wherein R^{A6} is methyl, ethyl, n-propyl, isopropyl, n-butyl, or benzyl. In some embodiments is a compound of Formula (Ii), or a pharmaceutically acceptable salt or solvate thereof, wherein R^{A6} is hydrogen, methyl, ethyl, n-propyl, isopropyl, n-butyl, or benzyl. In some embodiments is a compound of Formula (Ii), or a pharmaceutically acceptable salt or solvate thereof, wherein R⁵ is alkyl, and R^{A6} is hydrogen, methyl, ethyl, n-propyl, isopropyl, n-butyl, or benzyl. In some embodiments is a compound of Formula (Ii), or a pharmaceutically acceptable salt or solvate thereof, wherein R⁵ is unsubstituted alkyl, and R^{A6} is hydrogen, methyl, ethyl, n-propyl, isopropyl, n-butyl, or benzyl. In some embodiments is a compound of Formula (Ii), or a pharmaceutically acceptable salt or solvate thereof, wherein R⁵ is hydrogen, and R^{A6} is hydrogen, methyl, ethyl, n-propyl, isopropyl, n-butyl, or benzyl.

In some embodiments is a compound of Formula (Ii), or a pharmaceutically acceptable salt or solvate thereof, wherein R¹⁰ is hydrogen. In some embodiments is a compound of Formula (Ii), or a pharmaceutically acceptable salt or solvate thereof, wherein R¹⁰ is alkyl. In some embodiments is a compound of Formula (Ii), or a pharmaceutically acceptable salt or solvate thereof, wherein R¹⁰ is hydrogen, methyl, ethyl, n-propyl, or isopropyl. In some embodiments is a compound of Formula (Ii), or a pharmaceutically acceptable salt or solvate thereof, wherein R¹⁰ is CH₂CH₂OMe or CH₂CH₂SO₂Me.

In some embodiments is a compound of Formula (Ii), or a pharmaceutically acceptable salt or solvate thereof, wherein each of R⁵, R¹⁰, and R^{A6} is independently hydrogen, alkyl, heteroalkyl, or cycloalkyl. In some embodiments is a compound of Formula (Ii), or a pharmaceutically acceptable salt or solvate thereof, wherein each of R⁵, R¹⁰, and R^{A6} is independently hydrogen, alkyl, or cycloalkyl. In some embodiments is a compound of Formula (Ii), or a pharmaceutically acceptable salt or solvate thereof, wherein each of R⁵, R¹⁰, and R^{A6} is hydrogen. In some embodiments is a compound of Formula (Ii), or a pharmaceutically acceptable salt or solvate thereof, wherein each of R⁵, R¹⁰, and R^{A6} is independently hydrogen, methyl, ethyl, n-propyl, or isopropyl.

In some embodiments is a compound of Formula (Ii), or a pharmaceutically acceptable salt or solvate thereof, wherein the compound is:

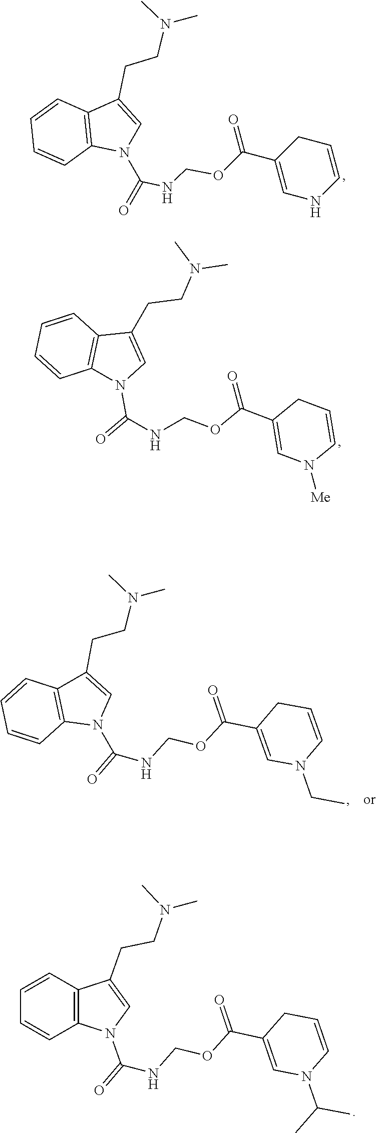 [SMILES: CN(C)CCc1cn(C(=O)NCOC(=O)C2=CNC=CC2)c2ccccc12 ; CN1C=CCC(C(=O)OCNC(=O)n2cc(CCN(C)C)c3ccccc32)=C1 ; CCN1C=CCC(C(=O)OCNC(=O)n2cc(CCN(C)C)c3ccccc32)=C1 ; CC(C)N1C=CCC(C(=O)OCNC(=O)n2cc(CCN(C)C)c3ccccc32)=C1]

In some embodiments is a compound of Formula (Ii), or a pharmaceutically acceptable salt or solvate thereof, wherein the compound is:

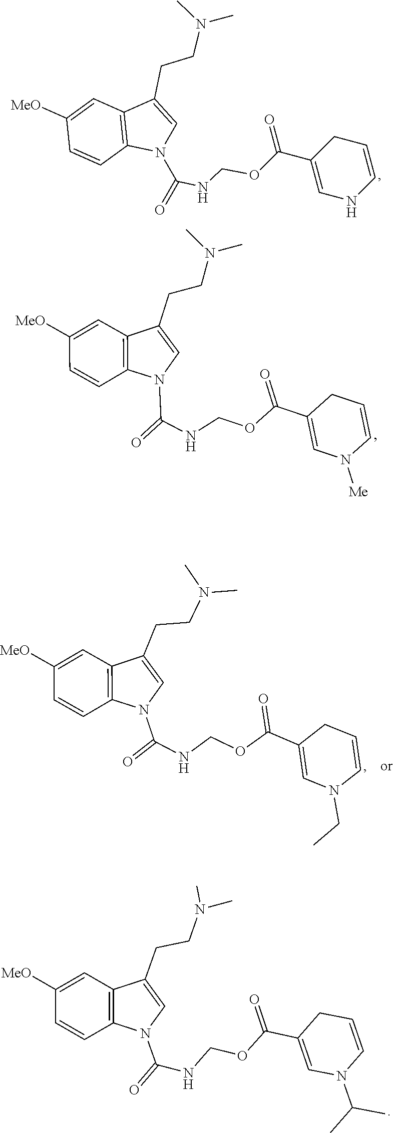

In some embodiments is a compound of Formula (I) having the structure of Formula (Ij), or a pharmaceutically acceptable salt thereof:

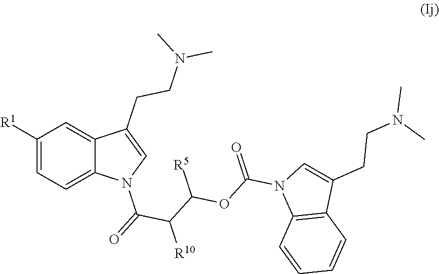

wherein R¹ is hydrogen or methoxy, and each of R⁵ and R¹⁰ is hydrogen, alkyl, or heteroalkyl, wherein each of alkyl and heteroalkyl is independently unsubstituted or substituted with one or more R^{A}.

In some embodiments is a compound of Formula (Ij), or a pharmaceutically acceptable salt or solvate thereof, wherein R⁵ is hydrogen. In some embodiments is a compound of Formula (Ij), or a pharmaceutically acceptable salt or solvate thereof, wherein R⁵ is alkyl. In some embodiments is a compound of Formula (Ij), or a pharmaceutically acceptable salt or solvate thereof, wherein R⁵ is unsubstituted alkyl. In some embodiments is a compound of Formula (Ij), or a pharmaceutically acceptable salt or solvate thereof, wherein R⁵ is methyl, ethyl, or isopropyl.

In some embodiments is a compound of Formula (Ij), or a pharmaceutically acceptable salt or solvate thereof, wherein R¹⁰ is hydrogen. In some embodiments is a compound of Formula (Ij), or a pharmaceutically acceptable salt or solvate thereof, wherein R¹⁰ is hydrogen, methyl, ethyl, n-propyl, or isopropyl. In some embodiments is a compound of Formula (Ij), or a pharmaceutically acceptable salt or solvate thereof, wherein R¹⁰ is CH₂CH₂OMe or CH₂CH₂SO₂Me.

In some embodiments is a compound of Formula (I) or (Ij), or a pharmaceutically acceptable salt or solvate thereof, wherein the compound is:

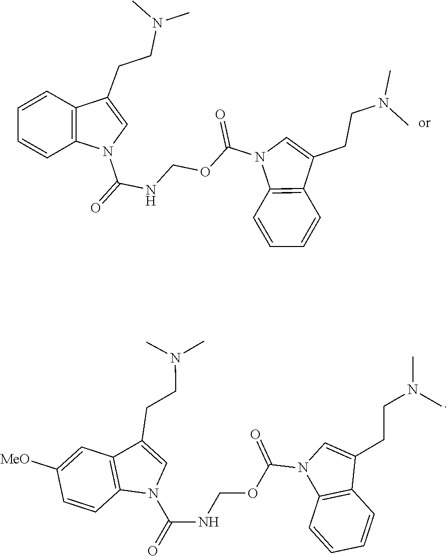 [SMILES: CN(C)CCc1cn(C(=O)NCOC(=O)n2cc(CCN(C)C)c3ccccc32)c2ccccc12 ; COc1ccc2c(c1)c(CCN(C)C)cn2C(=O)NCOC(=O)n1cc(CCN(C)C)c2ccccc21]

In some embodiments is a compound of Formula (I) having the structure of Formula (Ik), or a pharmaceutically acceptable salt thereof:

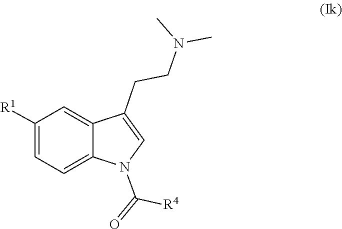

wherein R¹ is hydrogen or methoxy, and R⁴ is alkyl, heterocyclylalkyl, aryl, heteroaryl, or heteroalkyl, each of which is unsubstituted or substituted with one or more R^{A}.

In some embodiments is a compound of Formula (Ik), or a pharmaceutically acceptable salt or solvate thereof, wherein R⁴ is heteroalkyl. In some embodiments is a compound of Formula (Ik), or a pharmaceutically acceptable salt or solvate thereof, wherein R⁴ is heterocyclylalkyl. In some embodiments is a compound of Formula (Ik), or a pharmaceutically acceptable salt or solvate thereof, wherein R¹ is hydrogen and R⁴ is heteroalkyl. In some embodiments is a compound of Formula (Ik), or a pharmaceutically acceptable salt or solvate thereof, wherein R¹ is hydrogen and R⁴ is heterocyclylalkyl. In some embodiments is a compound of Formula (Ik), or a pharmaceutically acceptable salt or solvate thereof, wherein R¹ is methoxy and R⁴ is heteroalkyl. In some embodiments is a compound of Formula (Ik), or a pharmaceutically acceptable salt or solvate thereof, wherein R¹ is methoxy and R⁴ is heterocyclylalkyl.

In some embodiments is a compound of Formula (Ik), or a pharmaceutically acceptable salt or solvate thereof, wherein R⁴ is alkyl. In some embodiments is a compound of Formula (Ik), or a pharmaceutically acceptable salt or solvate thereof, wherein R⁴ is CH₂CF₃. In some embodiments is a compound of Formula (Ik), or a pharmaceutically acceptable salt or solvate thereof, wherein R⁴ is unsubstituted alkyl. In some embodiments is a compound of Formula (Ik), or a pharmaceutically acceptable salt or solvate thereof, wherein R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments is a compound of Formula (Ik), or a pharmaceutically acceptable salt or solvate thereof, wherein R⁴ is cycloalkyl. In some embodiments is a compound of Formula (Ik), or a pharmaceutically acceptable salt or solvate thereof, wherein R⁴ is unsubstituted cycloalkyl. In some embodiments is a compound of Formula (Ik), or a pharmaceutically acceptable salt or solvate thereof, wherein R⁴ is cyclopropyl, cyclobutyl, cyclohexyl, cycloheptyl, or cyclooctyl. In some embodiments is a compound of Formula (Ik), or a pharmaceutically acceptable salt or solvate thereof, wherein R⁴ is aryl. In some embodiments is a compound of Formula (Ik), or a pharmaceutically acceptable salt or solvate thereof, wherein R⁴ is phenyl. In some embodiments is a compound of Formula (Ik), or a pharmaceutically acceptable salt or solvate thereof, wherein R⁴ is heteroaryl. In some embodiments is a compound of Formula (Ik), or a pharmaceutically acceptable salt or solvate thereof, wherein R⁴ is 2-pyridyl, 3-pyridyl, 4-pyridyl, 2-pyrimidyl, 3-pyrimidyl, or 6-pyrimidyl.

In some embodiments is a compound of Formula (I) or (Ik), or a pharmaceutically acceptable salt or solvate thereof, wherein the compound is:

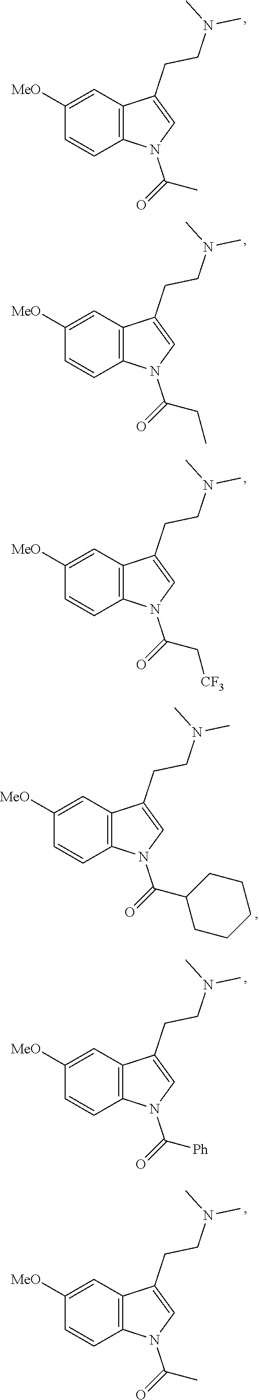

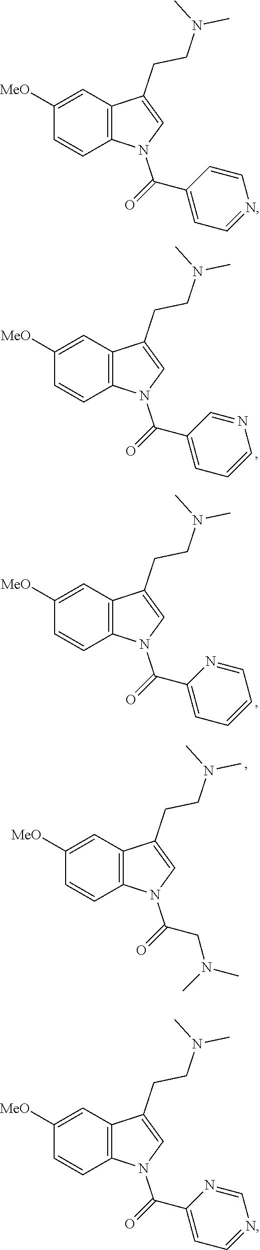

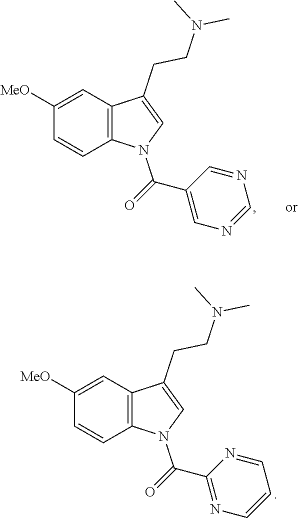 [SMILES: COc1ccc2c(c1)c(CCN(C)C)cn2C(=O)c1cncnc1 ; COc1ccc2c(c1)c(CCN(C)C)cn2C(=O)c1ncccn1]

In some embodiments is a compound of Formula (Ik), or a pharmaceutically

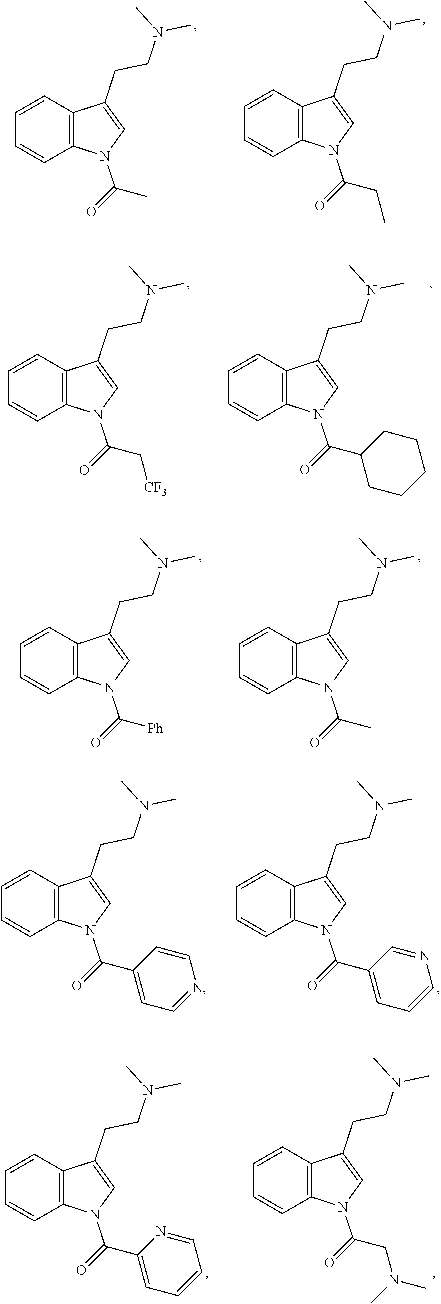

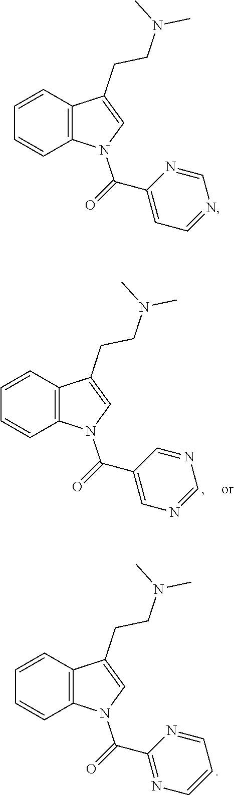 [SMILES: CN(C)CCc1cn(C(=O)c2ccncn2)c2ccccc12 ; CN(C)CCc1cn(C(=O)c2cncnc2)c2ccccc12 ; CN(C)CCc1cn(C(=O)c2ncccn2)c2ccccc12]

In some embodiments is a compound of Formula (I) or (Ik), or a pharmaceutically acceptable salt or solvate thereof, wherein the compound is:

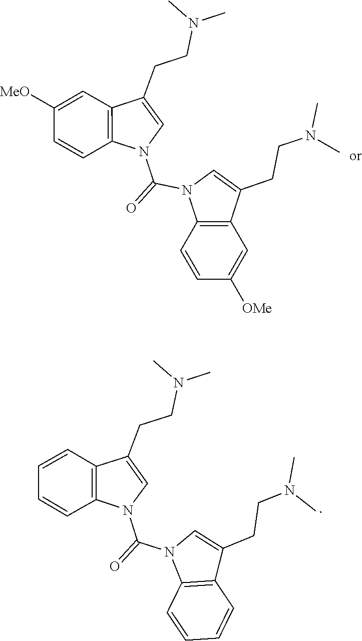 [SMILES: COc1ccc2c(c1)c(CCN(C)C)cn2C(=O)n1cc(CCN(C)C)c2cc(OC)ccc21 ; CN(C)CCc1cn(C(=O)n2cc(CCN(C)C)c3ccccc32)c2ccccc12]

In some embodiments is a compound of Formula (I) or (Ik), or a pharmaceutically acceptable salt or solvate thereof, wherein the compound is:

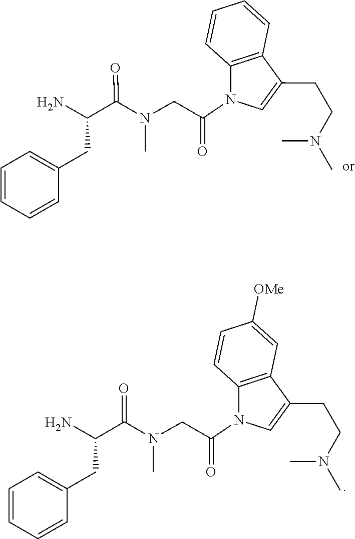 [SMILES: CN(C)CCc1cn(C(=O)CN(C)C(=O)[C@@H](N)Cc2ccccc2)c2ccccc12 ; COc1ccc2c(c1)c(CCN(C)C)cn2C(=O)CN(C)C(=O)[C@@H](N)Cc1ccccc1]

In some embodiments is a compound of Formula (Ik), or a pharmaceutically acceptable salt or solvate thereof, wherein R⁴ is heterocyclylalkyl. In some embodiments is a compound of Formula (Ik), or a pharmaceutically acceptable salt or solvate thereof, wherein R⁴ is

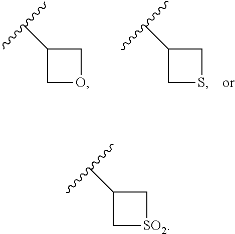 [SMILES: *C1COC1 ; *C1CSC1 ; *C1CS(=O)(=O)C1]

In some embodiments is a compound of Formula (Ik), or a pharmaceutically acceptable salt or solvate thereof, wherein the compound is:

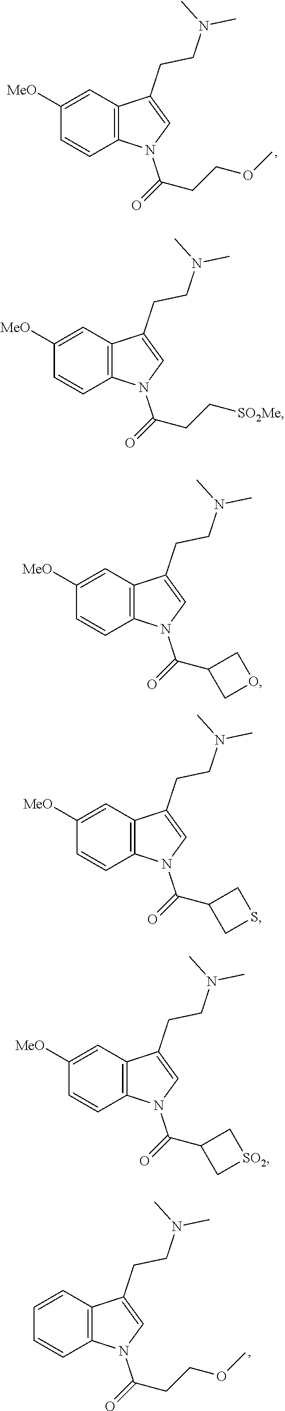

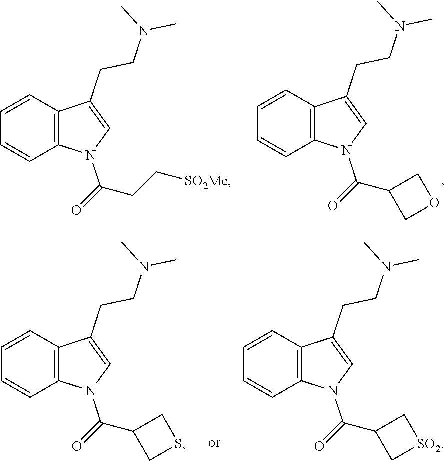

In some embodiments is a compound of Formula (Ik), or a pharmaceutically acceptable salt or solvate thereof, wherein R⁴ is

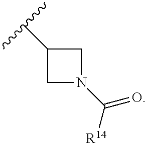

In some embodiments is a compound of Formula (Ik), or a pharmaceutically acceptable salt or solvate thereof, wherein R⁴ is

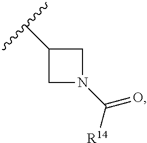

wherein R¹⁴ is alkyl, cycloalkyl, or aryl, each of which is independently unsubstituted or substituted with one or more R^{B}. In some embodiments is a compound of Formula (Ik), or a pharmaceutically acceptable salt or solvate thereof, wherein R⁴ is

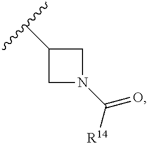

wherein R¹⁴ is methyl, ethyl, n-propyl, isopropyl, or CH₂CH₂OMe. In some embodiments is a compound of Formula (Ik), or a pharmaceutically acceptable salt or solvate thereof, wherein R⁴ is

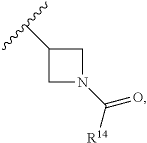

wherein R¹⁴ is phenyl.

In some embodiments is a compound of Formula (I) or (Ik), or a pharmaceutically acceptable salt or solvate thereof, wherein the compound is:

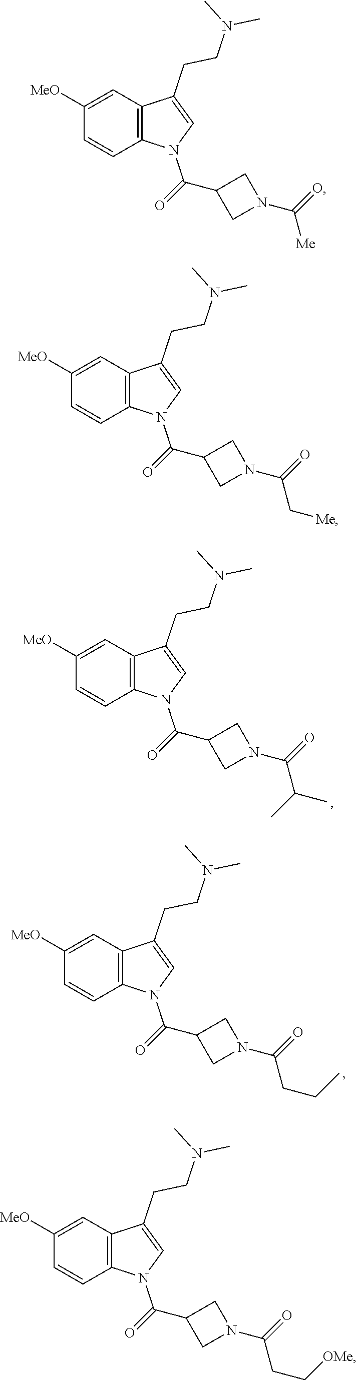 [SMILES: COc1ccc2c(c1)c(CCN(C)C)cn2C(=O)C1CN(C(C)=O)C1 ; CCC(=O)N1CC(C(=O)n2cc(CCN(C)C)c3cc(OC)ccc32)C1 ; COc1ccc2c(c1)c(CCN(C)C)cn2C(=O)C1CN(C(=O)C(C)C)C1 ; CCCC(=O)N1CC(C(=O)n2cc(CCN(C)C)c3cc(OC)ccc32)C1 ; COCCC(=O)N1CC(C(=O)n2cc(CCN(C)C)c3cc(OC)ccc32)C1]

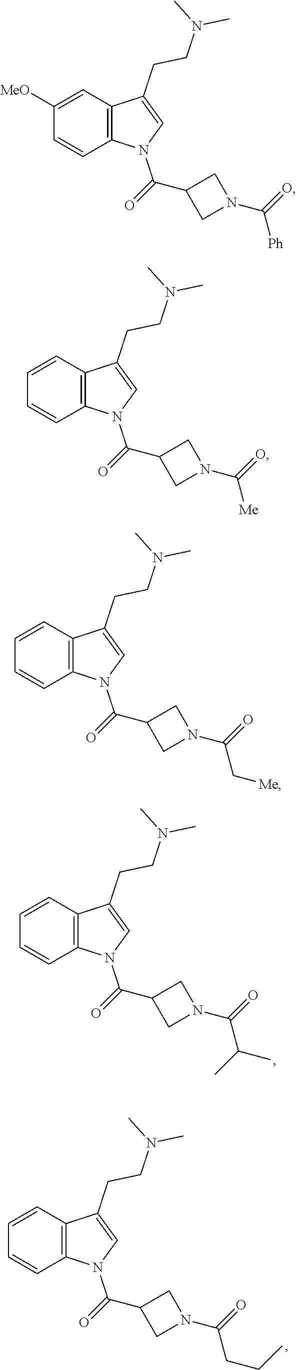 [SMILES: COc1ccc2c(c1)c(CCN(C)C)cn2C(=O)C1CN(C(=O)c2ccccc2)C1 ; CC(=O)N1CC(C(=O)n2cc(CCN(C)C)c3ccccc32)C1 ; CCC(=O)N1CC(C(=O)n2cc(CCN(C)C)c3ccccc32)C1 ; CC(C)C(=O)N1CC(C(=O)n2cc(CCN(C)C)c3ccccc32)C1 ; CCCC(=O)N1CC(C(=O)n2cc(CCN(C)C)c3ccccc32)C1]

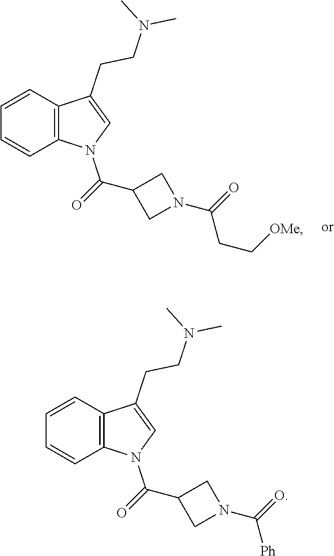 [SMILES: COCCC(=O)N1CC(C(=O)n2cc(CCN(C)C)c3ccccc32)C1 ; "or" ; CN(C)CCc1cn(C(=O)C2CN(C(=O)c3ccccc3)C2)c2ccccc12]

In some embodiments is a compound of Formula (Ik), or a pharmaceutically acceptable salt or solvate thereof, wherein R⁴ is

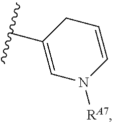

wherein R^{A7} is hydrogen or alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹. In some embodiments is a compound of Formula (Ik), or a pharmaceutically acceptable salt or solvate thereof, wherein R⁴ is

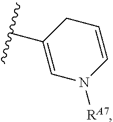

wherein R^{A7} is hydrogen. In some embodiments is a compound of Formula (Ik), or a pharmaceutically acceptable salt or solvate thereof, wherein R⁴ is

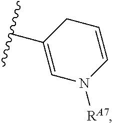

wherein R^{A7} is alkyl that is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹. In some embodiments is a compound of Formula (Ik), or a pharmaceutically acceptable salt or solvate thereof, wherein R⁴ is

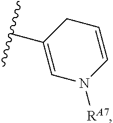

wherein R^{A7} is unsubstituted alkyl. In some embodiments is a compound of Formula (Ik), or a pharmaceutically acceptable salt or solvate thereof, wherein R⁴ is

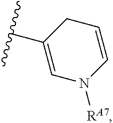

wherein R^{A7} is methyl, ethyl, n-propyl, isopropyl, or n-butyl. In some embodiments is a compound of Formula (Ik), or a pharmaceutically acceptable salt or solvate thereof, wherein R⁴ is

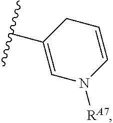

wherein R^{A7} is benzyl.

In some embodiments is a compound of Formula (Ik), or a pharmaceutically acceptable salt or solvate thereof, wherein the compound is:

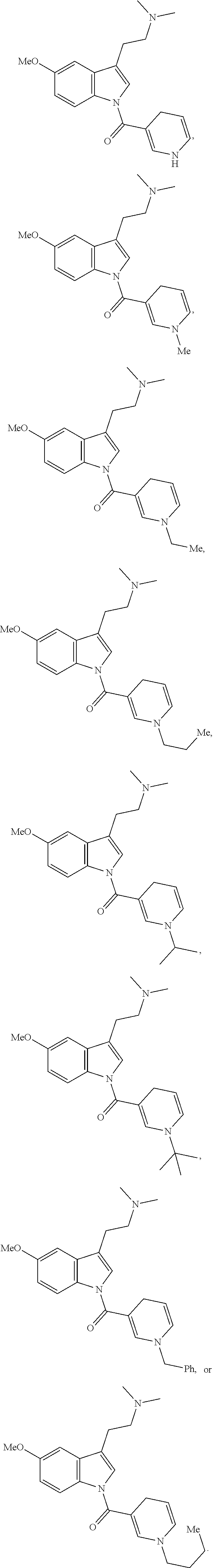

In some embodiments is a compound of Formula (Ik), or a pharmaceutically acceptable salt or solvate thereof, wherein the compound is:

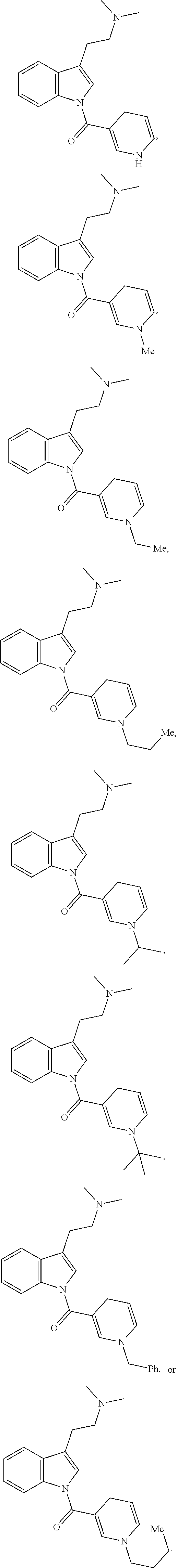

In some embodiments is a compound of Formula (I) or (Ik) having the structure of Formula (Ik1), or a pharmaceutically acceptable salt thereof:

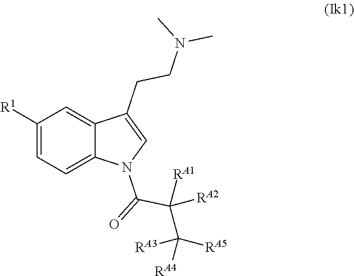

wherein:
R¹ is methoxy or hydrogen;
each of R^{A1}, R^{A2}, R^{A3}, and R^{A4} is independently hydrogen, alkyl, or an amino acid side chain, wherein each alkyl or amino acid side chain is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R¹⁰ is hydrogen, alkyl, heteroalkyl, or cycloalkyl, wherein each of alkyl, heteroalkyl, and cycloalkyl is unsubstituted or substituted with one or more R^{A}; and
R^{A5} is heteroalkyl, heterocyclylalkyl, heteroaryl, —C(O)OR¹³, —N(R¹³)C(O)OR¹⁴, —N(R¹³)C(O)R¹⁴, —C(O)R¹⁴, —OC(O)R⁵, or —OC(O)OR¹⁶, wherein each of heteroalkyl, heterocyclylalkyl, heteroaryl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Ik1), or a pharmaceutically acceptable salt or solvate thereof, wherein each of R^{A1}, R^{A2}, R^{A3}, and R^{A4} is hydrogen. In some embodiments is a compound of Formula (Ik1), or a pharmaceutically acceptable salt or solvate thereof, wherein each of R^{A1}, R^{A2}, R^{A3}, and R^{A4} is hydrogen or alkyl. In some embodiments is a compound of Formula (Ik1), or a pharmaceutically acceptable salt or solvate thereof, wherein each of R^{A1}, R^{A2}, R^{A3}, and R^{A4} is hydrogen or unsubstituted alkyl. In some embodiments is a compound of Formula (Ik1), or a pharmaceutically acceptable salt or solvate thereof, wherein R^{A5} is heteroalkyl or heterocyclylalkyl. In some embodiments is a compound of Formula (Ik1), or a pharmaceutically acceptable salt or solvate thereof, wherein R^{A5} is heterocyclylalkyl. In some embodiments is a compound of Formula (Ik1), or a pharmaceutically acceptable salt or solvate thereof, wherein R^{A5} is heteroalkyl. In some embodiments is a compound of Formula (Ik1), or a pharmaceutically acceptable salt or solvate thereof, wherein each of R^{A1}, R^{A2}, R^{A3}, and R^{A4} is hydrogen, and R^{A5} is alkoxy. In some embodiments is a compound of Formula (Ik1), or a pharmaceutically acceptable salt or solvate thereof, wherein each of R^{A1}, R^{A2}, R^{A3}, and R^{A4} is hydrogen, and R^{A5} is methoxy. In some embodiments is a compound of Formula (Ik1), or a pharmaceutically acceptable salt or solvate thereof, wherein each of R^{A1}, R^{A2}, R^{A3}, and R^{A4} is hydrogen, and R^{A5} is alkylsulfonyl. In some embodiments is a compound of Formula (Ik1), or a pharmaceutically acceptable salt or solvate thereof, wherein each of R^{A1}, R^{A2}, R^{A3}, and R^{A4} is hydrogen, and R^{A5} is methylsulfonyl.

In some embodiments is a compound of Formula (Ik1), or a pharmaceutically acceptable salt or solvate thereof, wherein R^{A5} is —OC(O)R¹⁵. In some embodiments is a compound of Formula (Ik1), or a pharmaceutically acceptable salt or solvate thereof, wherein R^{A5} is —OC(O)R¹⁵, wherein R¹⁵ is alkyl, cycloalkyl, aryl, heteroaryl, heteroalkyl, or heterocyclylalkyl. In some embodiments is a compound of Formula (Ik1), or a pharmaceutically acceptable salt or solvate thereof, wherein R^{A5} is —OC(O)R¹⁵, wherein R¹⁵ is alkyl. In some embodiments is a compound of Formula (Ik1), or a pharmaceutically acceptable salt or solvate thereof, wherein R^{A5} is —OC(O)R¹⁵, wherein R¹⁵ is unsubstituted alkyl. In some embodiments is a compound of Formula (Ik1), or a pharmaceutically acceptable salt or solvate thereof, wherein R^{A5} is —OC(O)R¹⁵, wherein R¹⁵ is methyl, ethyl, n-propyl, isopropyl n-butyl, tert-butyl, n-pentyl, or 3-methyl-1-butyl. In some embodiments is a compound of Formula (Ik1), or a pharmaceutically acceptable salt or solvate thereof, wherein R^{A5} is —OC(O)R¹⁵, wherein R¹⁵ is aryl. In some embodiments is a compound of Formula (Ik1), or a pharmaceutically acceptable salt or solvate thereof, wherein R^{A5} is —OC(O)R¹⁵, wherein R¹⁵ is unsubstituted aryl. In some embodiments is a compound of Formula (Ik1), or a pharmaceutically acceptable salt or solvate thereof, wherein R^{A5} is —OC(O)R¹⁵, wherein R¹⁵ is phenyl. In some embodiments is a compound of Formula (Ik1), or a pharmaceutically acceptable salt or solvate thereof, wherein R^{A5} is —OC(O)R¹⁵, wherein R¹⁵ is 2-pyridyl, 3-pyridyl, 4-pyridyl, 2-pyrimidyl, 4-pyrimidyl, 5-pyrimidyl, or 6-pyrimidyl.

In some embodiments is a compound of Formula (I) or (Ik) having the structure of Formula (Ik2), or a pharmaceutically acceptable salt thereof:

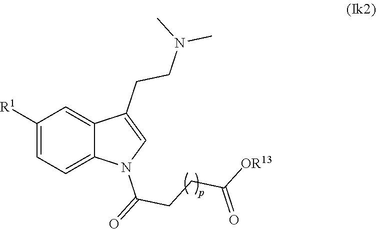

wherein:
R¹ is methoxy or hydrogen;
R¹³ is hydrogen, alkyl, cycloalkyl, aryl, heteroaryl, heteroalkyl, or heterocyclylalkyl, wherein each of alkyl, cycloalkyl, aryl, heteroaryl, heteroalkyl, and heterocyclylalkyl is unsubstituted or substituted with one or more R^{B}; and
p is 0, 1, 2, 3, 4, 5, 6, 7, 8, 9, or 10.

In some embodiments is a compound of Formula (I), (Ik) or (Ik2), or a pharmaceutically acceptable salt or solvate thereof, wherein R¹³ is alkyl, cycloalkyl, aryl, heteroaryl, heteroalkyl, or heterocyclylalkyl. In some embodiments is a compound of Formula (I), (Ik) or (Ik2), or a pharmaceutically acceptable salt or solvate thereof, wherein R¹³ is hydrogen or alkyl. In some embodiments is a compound of Formula (I), (Ik) or (Ik2), or a pharmaceutically acceptable salt or solvate thereof, wherein R¹³ is hydrogen. In some embodiments is a compound of Formula (I), (Ik) or (Ik2), or a pharmaceutically acceptable salt or solvate thereof, wherein R¹³ is alkyl. In some embodiments is a compound of Formula (I), (Ik) or (Ik2), or a pharmaceutically acceptable salt or solvate thereof, wherein R¹³ is unsubstituted alkyl. In some embodiments is a compound of Formula (I), (Ik) or (Ik2), or a pharmaceutically acceptable salt or solvate thereof, wherein R¹³ is methyl, ethyl, n-propyl, isopropyl n-butyl, tert-butyl, n-pentyl, or 3-methyl-1-butyl. In some embodiments is a compound of Formula (I), (Ik) or (Ik2), or a pharmaceutically acceptable salt or solvate thereof, wherein R¹³ is hydrogen, methyl, ethyl, n-propyl, isopropyl n-butyl, tert-butyl, n-pentyl, or 3-methyl-1-butyl. In some embodiments is a compound of Formula (I), (Ik) or (Ik2), or a pharmaceutically acceptable salt or solvate thereof, wherein R¹³ is methyl. In some embodiments is a compound of Formula (I), (Ik) or (Ik2), or a pharmaceutically acceptable salt or solvate thereof, wherein R¹³ is hydrogen or methyl. In some embodiments is a compound of Formula (I), (Ik) or (Ik2), or a pharmaceutically acceptable salt or solvate thereof, wherein R¹³ is aryl. In some embodiments is a compound of Formula (I), (Ik) or (Ik2), or a pharmaceutically acceptable salt or solvate thereof, wherein R¹³ is unsubstituted aryl. In some embodiments is a compound of Formula (I), (Ik) or (Ik2), or a pharmaceutically acceptable salt or solvate thereof, wherein R¹³ is phenyl. In some embodiments is a compound of Formula (I), (Ik) or (Ik2), or a pharmaceutically acceptable salt or solvate thereof, wherein R¹³ is 2-pyridyl, 3-pyridyl, 4-pyridyl, 2-pyrimidyl, 4-pyrimidyl, 5-pyrimidyl, or 6-pyrimidyl. In some embodiments is a compound of Formula (I), (Ik) or (Ik2), or a pharmaceutically acceptable salt or solvate thereof, wherein p is 1, 2, 3, 4, or 5. In some embodiments is a compound of Formula (I), (Ik) or (Ik2), or a pharmaceutically acceptable salt or solvate thereof, wherein p is 1. In some embodiments is a compound of Formula (I), (Ik) or (Ik2), or a pharmaceutically acceptable salt or solvate thereof, wherein p is 2. In some embodiments is a compound of Formula (I), (Ik) or (Ik2), or a pharmaceutically acceptable salt or solvate thereof, wherein p is 3. In some embodiments is a compound of Formula (I), (Ik) or (Ik2), or a pharmaceutically acceptable salt or solvate thereof, wherein p is 4. In some embodiments is a compound of Formula (I), (Ik) or (Ik2), or a pharmaceutically acceptable salt or solvate thereof, wherein p is 5.

In some embodiments is a compound of Formula (Ik1), or a pharmaceutically acceptable salt or solvate thereof, wherein the compound is:

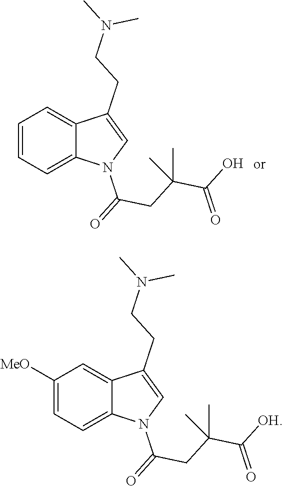

In some embodiments is a compound of Formula (Ik2), or a pharmaceutically acceptable salt or solvate thereof, wherein the compound is:

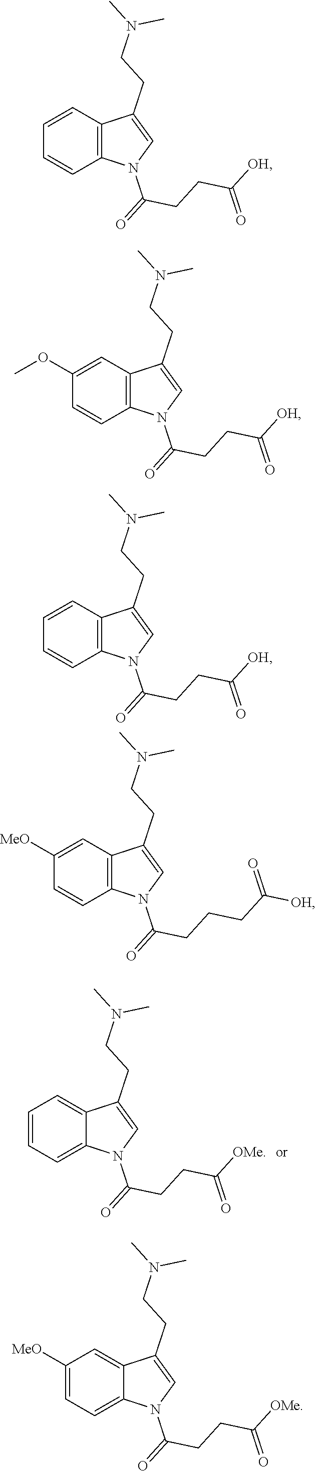

In some embodiments is a compound of Formula (I) or (Ik) having the structure of Formula (Ik3), or a pharmaceutically acceptable salt thereof:

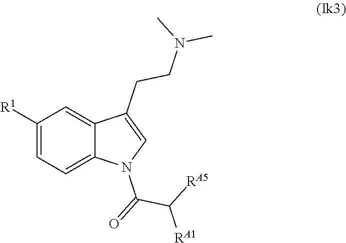

wherein:
R¹ is methoxy or hydrogen;
R^{A1} is alkyl or an amino acid side chain, each of which is unsubstituted or substituted with one or more alkyl, aryl, halogen, —OR¹³, —NR(R¹⁸)R¹⁹, —C(O)R¹⁴, —OC(O)R⁵, —OC(O)OR¹⁶, or —OC(O)N(R¹⁸)R¹⁹; and
R^{A5} is —N(R¹⁸)R¹⁹ or —N(R¹³)C(O)R¹⁴.

In some embodiments is a compound of Formula (Ik3), or a pharmaceutically acceptable salt or solvate thereof, wherein R^{A5} is —N(R¹⁸)R¹⁹. In some embodiments is a compound of Formula (Ik3), or a pharmaceutically acceptable salt or solvate thereof, wherein R^{A1} is —N(R¹⁸)R¹⁹, wherein each of R¹⁸ and R¹⁹ is hydrogen. In some embodiments is a compound of Formula (Ik3), or a pharmaceutically acceptable salt or solvate thereof, wherein R^{A5} is —N(R¹⁸)R¹⁹, wherein R¹⁹ is alkyl, cycloalkyl, or aryl. In some embodiments is a compound of Formula (Ik3), or a pharmaceutically acceptable salt or solvate thereof, wherein R^{A5} is —N(R¹⁸)R¹⁹, wherein R¹⁸ is hydrogen, and R¹⁹ is alkyl, cycloalkyl, or aryl. In some embodiments is a compound of Formula (Ik3), or a pharmaceutically acceptable salt or solvate thereof, wherein R^{A5} is —N(R¹⁸)R¹⁹, wherein R¹⁸ is hydrogen, and R¹⁹ is unsubstituted alkyl, unsubstituted cycloalkyl, or unsubstituted aryl. In some embodiments is a compound of Formula (Ik3), or a pharmaceutically acceptable salt or solvate thereof, wherein R^{A5} is —N(R¹⁸)R¹⁹, wherein R¹⁸ is hydrogen, and R¹⁹ is methyl, ethyl, isopropyl, tert-butyl, or phenyl.

In some embodiments is a compound of Formula (Ik3), or a pharmaceutically acceptable salt or solvate thereof, wherein R^{A5} is —N(R¹³)C(O)R¹⁴.

In some embodiments is a compound of Formula (Ik3), or a pharmaceutically acceptable salt or solvate thereof, wherein the compound is:

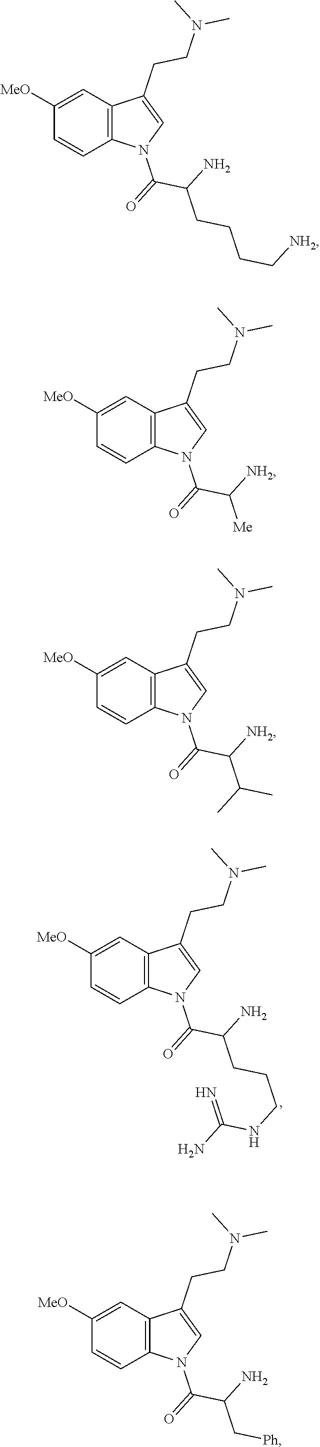

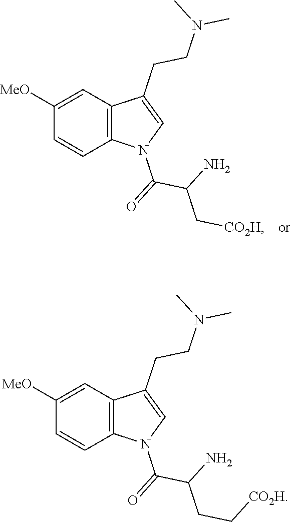 [SMILES: COc1ccc2c(c1)c(CCN(C)C)cn2C(=O)C(N)CC(=O)O ; COc1ccc2c(c1)c(CCN(C)C)cn2C(=O)C(N)CCC(=O)O]

In some embodiments is a compound of Formula (Ik3), or a pharmaceutically acceptable salt or solvate thereof, wherein the compound is:

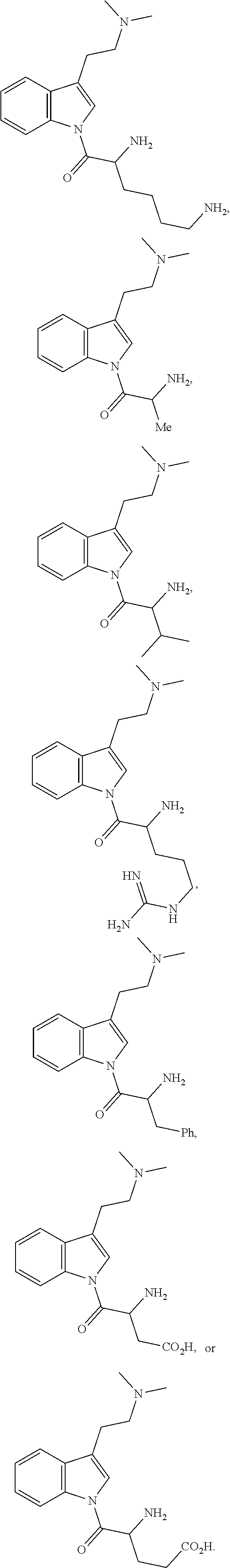

In some embodiments is a compound of Formula (Ik3), or a pharmaceutically acceptable salt or solvate thereof, wherein the compound is:

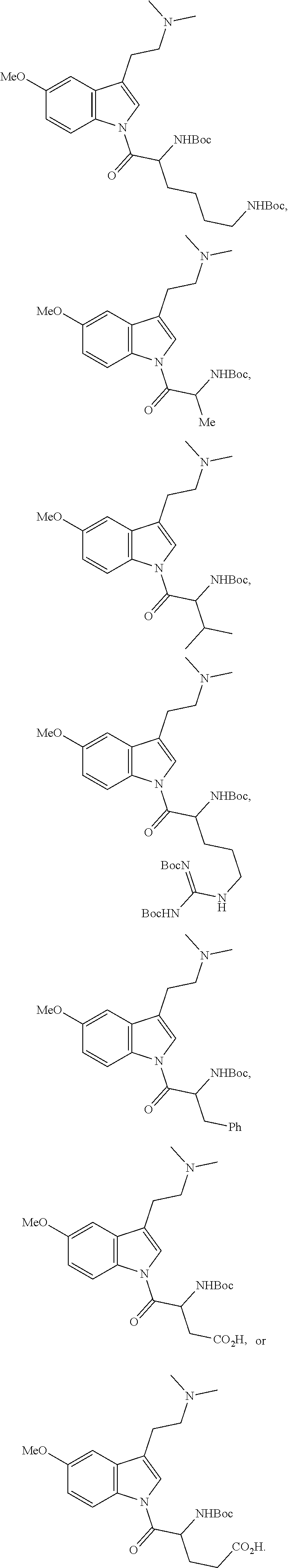

In some embodiments is a compound of Formula (Ik3), or a pharmaceutically acceptable salt or solvate thereof, wherein the compound is:

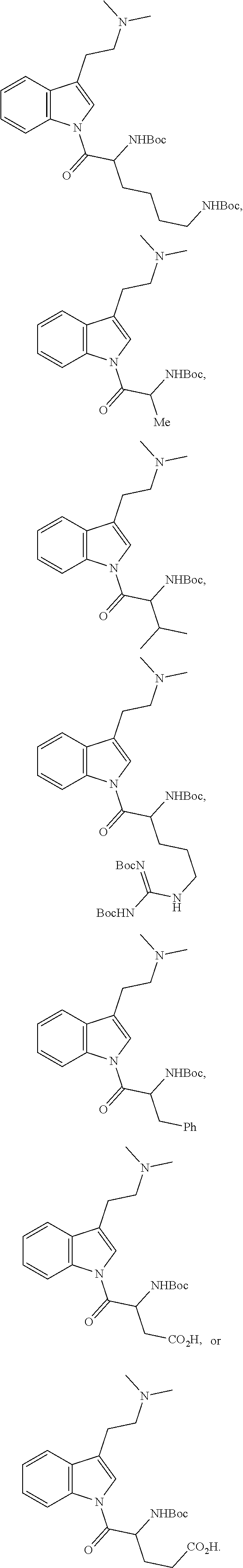

In some embodiments is a compound of Formula (I) having the structure of Formula (Il), or a pharmaceutically acceptable salt thereof:

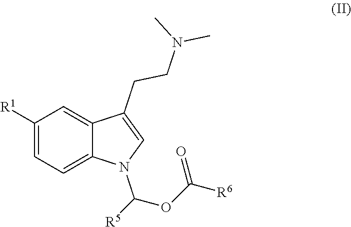

wherein:
R¹ is methoxy or hydrogen;
R⁵ is hydrogen, alkyl, or cycloalkyl, wherein each of alkyl or cycloalkyl is unsubstituted or substituted with one or more R^{A}; and
R⁶ is alkyl, cycloalkyl, heterocyclylalkyl, or heteroalkyl, wherein each of alkyl, cycloalkyl, heterocyclylalkyl, or heteroalkyl is unsubstituted or substituted with one or more R^{A}.

In some embodiments is a compound of Formula (I) or (Il), or a pharmaceutically acceptable salt or solvate thereof, wherein R⁶ is methyl, ethyl, isopropyl, tert-butyl, 2-dimethylaminoethyl, or cyclopropyl. In some embodiments is a compound of Formula (I) or (Il), or a pharmaceutically acceptable salt or solvate thereof, wherein R¹ is hydrogen, R⁵ is hydrogen, and R⁶ is methyl, ethyl, isopropyl, tert-butyl, 2-dimethylaminoethyl, or cyclopropyl. In some embodiments is a compound of Formula (I) or (Il), or a pharmaceutically acceptable salt or solvate thereof, wherein R¹ is methoxy, R⁵ is hydrogen, and R⁶ is methyl, ethyl, isopropyl, tert-butyl, 2-dimethylaminoethyl, or cyclopropyl. In some embodiments is a compound of Formula (I) or (Il), or a pharmaceutically acceptable salt or solvate thereof, wherein R¹ is hydrogen, R⁵ is hydrogen, and R⁶ is tert-butyl. In some embodiments is a compound of Formula (I) or (Il), or a pharmaceutically acceptable salt or solvate thereof, R⁵ is hydrogen. In some embodiments is a compound of Formula (I) or (Il), or a pharmaceutically acceptable salt or solvate thereof, wherein R¹ is methoxy, R⁵ is hydrogen, and R⁶ is tert-butyl.

In some embodiments is a compound of Formula (I) or (Il), or a pharmaceutically acceptable salt or solvate thereof, wherein the compound is:

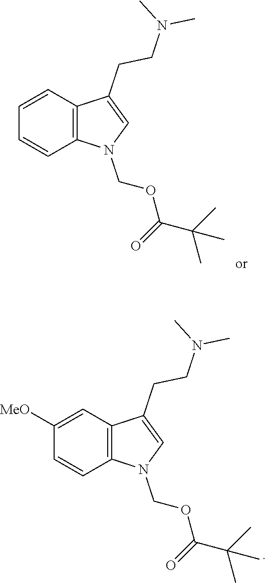 [SMILES: CN(C)CCc1cn(COC(=O)C(C)(C)C)c2ccccc12 ; COc1ccc2c(c1)c(CCN(C)C)cn2COC(=O)C(C)(C)C]

In some embodiments is a compound of Formula (I) having the structure of Formula (Im), or a pharmaceutically acceptable salt thereof:

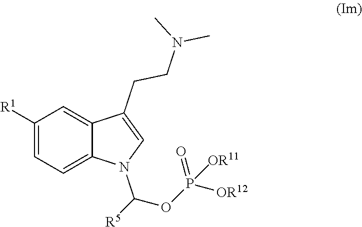

wherein:
R¹ is methoxy or hydrogen;
R⁵ hydrogen, alkyl, cycloalkyl, or heteroalkyl, wherein each of alkyl, cycloalkyl, and heteroalkyl is unsubstituted or substituted with one or more R^{A}; and
each of R¹¹ and R¹² is independently hydrogen, cycloalkyl, aryl, heteroaryl, or alkyl, wherein each of alkyl, cycloalkyl, and heteroalkyl is independently unsubstituted or substituted with one or more R^{A}.

In some embodiments is a compound of Formula (I) or (Im), or a pharmaceutically acceptable salt or solvate thereof, wherein each of R¹¹ and R¹² is independently cycloalkyl, aryl, heteroaryl, or alkyl. In some embodiments is a compound of Formula (I) or (Im), or a pharmaceutically acceptable salt or solvate thereof, wherein R¹¹ is hydrogen and R¹² is cycloalkyl, aryl, heteroaryl, or alkyl. In some embodiments is a compound of Formula (I) or (Im), or a pharmaceutically acceptable salt or solvate thereof, wherein R¹¹ is hydrogen and R¹² is alkyl. In some embodiments is a compound of Formula (I) or (Im), or a pharmaceutically acceptable salt or solvate thereof, wherein R¹¹ is hydrogen and R¹² is tert-butyl. In some embodiments is a compound of Formula (I) or (Im), or a pharmaceutically acceptable salt or solvate thereof, wherein R⁵ is hydrogen or alkyl. In some embodiments is a compound of Formula (I) or (Im), or a pharmaceutically acceptable salt or solvate thereof, wherein R⁵ is hydrogen, unsubstituted alkyl, unsubstituted cycloalkyl, unsubstituted heteroalkyl, or alkyl substituted with heteroaryl. In some embodiments is a compound of Formula (I) or (Im), or a pharmaceutically acceptable salt or solvate thereof, wherein each of R¹¹ and R¹² is independently selected from unsubstituted alkyl, unsubstituted cycloalkyl, unsubstituted aryl, unsubstituted heteroaryl, unsubstituted alkyl, or alkyl substituted with aryl or heteroaryl. In some embodiments is a compound of Formula (I) or (Im), or a pharmaceutically acceptable salt or solvate thereof, wherein R¹¹ is hydrogen, and R¹² is cycloalkyl, aryl, heteroaryl, or alkyl. In some embodiments is a compound of Formula (I) or (Im), or a pharmaceutically acceptable salt or solvate thereof, wherein R¹¹ is hydrogen, and R¹² is alkyl. In some embodiments is a compound of Formula (I) or (Im), or a pharmaceutically acceptable salt or solvate thereof, wherein each of R¹¹ and R¹² is alkyl. In some embodiments is a compound of Formula (I) or (Im), or a pharmaceutically acceptable salt or solvate thereof, wherein each of R¹¹ and R¹² is unsubstituted alkyl. In some embodiments is a compound of Formula (I) or (Im), or a pharmaceutically acceptable salt or solvate thereof, wherein each of R¹¹ and R¹² is alkyl substituted with —OC(O)R¹⁵. In some embodiments is a compound of Formula (I) or (Im), or a pharmaceutically acceptable salt or solvate thereof, wherein each of R¹¹ and R¹² is alkyl substituted with —OC(O)R¹⁵, wherein each R¹⁵ is alkyl, cycloalkyl, heterocyclylalkyl, aryl, or heteroaryl. In some embodiments is a compound of Formula (I) or (Im), or a pharmaceutically acceptable salt or solvate thereof, wherein each of R¹¹ and R¹² is alkyl substituted with —OC(O)R¹⁵, wherein each R¹⁵ is unsubstituted alkyl, unsubstituted cycloalkyl, unsubstituted heterocyclylalkyl, unsubstituted aryl, or unsubstituted heteroaryl. In some embodiments is a compound of Formula (I) or (Im), or a pharmaceutically acceptable salt or solvate thereof, wherein each of R¹¹ and R¹² is alkyl substituted with —OC(O)R¹⁵, wherein each R¹⁵ is heterocyclylalkyl substituted with alkyl or arylalkyl.

In some embodiments is a compound of Formula (I) or (Im), or a pharmaceutically acceptable salt or solvate thereof, wherein R⁴ is hydrogen. In some embodiments is a compound of Formula (I) or (Im), or a pharmaceutically acceptable salt or solvate thereof, wherein R⁵ is hydrogen and each of R¹¹ and R¹² is alkyl, heterocyclylalkyl, or cycloalkyl. In some embodiments is a compound of Formula (I) or (Im), or a pharmaceutically acceptable salt or solvate thereof, wherein R⁵ is hydrogen. In some embodiments is a compound of Formula (I) or (Im), or a pharmaceutically acceptable salt or solvate thereof, wherein R⁵ is hydrogen and each of R¹¹ and R¹² is alkyl. In some embodiments is a compound of Formula (I) or (Im), or a pharmaceutically acceptable salt or solvate thereof, wherein R⁵ is hydrogen and each of R¹¹ and R¹² is unsubstituted alkyl. In some embodiments is a compound of Formula (I) or (Im), or a pharmaceutically acceptable salt or solvate thereof, wherein R¹ is methoxy, R⁴ is hydrogen, and each of R¹¹ and R¹² is unsubstituted alkyl. In some embodiments is a compound of Formula (I) or (Im), or a pharmaceutically acceptable salt or solvate thereof, wherein R¹ is hydrogen, R⁵ is hydrogen, and each of R¹¹ and R¹² is unsubstituted alkyl. In some embodiments is a compound of Formula (I) or (Im), or a pharmaceutically acceptable salt or solvate thereof, wherein R¹ is methoxy, R⁵ is hydrogen, and each of R¹¹ and R¹² is tert-butyl. In some embodiments is a compound of Formula (I) or (Im), or a pharmaceutically acceptable salt or solvate thereof, wherein R¹ is hydrogen, R⁵ is hydrogen, and each of R¹¹ and R¹² is tert-butyl.

In some embodiments is a compound of Formula (I) or (Im), or a pharmaceutically acceptable salt or solvate thereof, wherein each of R¹¹ and R¹² is

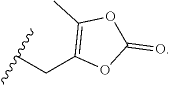 [SMILES: *Cc1oc(=O)oc1C]

In some embodiments is a compound of Formula (I) or (Im), or a pharmaceutically acceptable salt or solvate thereof, wherein each of R¹¹ and R¹² is methyl, ethyl, n-propyl, isopropyl, n-butyl, tert-butyl, n-pentyl, n-hexyl, or 3-methyl-1-butyl. In some embodiments is a compound of Formula (I) or (Im), or a pharmaceutically acceptable salt or solvate thereof, wherein each of R¹¹ and R¹² is cyclopropyl, cyclobutyl, cyclopentyl, cyclohexyl, or cycloheptyl. In some embodiments is a compound of Formula (I) or (Im), or a pharmaceutically acceptable salt or solvate thereof, wherein each of R¹¹ and R¹² is phenyl. In some embodiments is a compound of Formula (I) or (Im), or a pharmaceutically acceptable salt or solvate thereof, wherein each of R¹¹ and R¹² is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (Im), or a pharmaceutically acceptable salt or solvate thereof, wherein each of R¹¹ and R¹² is 4-nitrophenyl. In some embodiments is a compound of Formula (I) or (Im), or a pharmaceutically acceptable salt or solvate thereof, wherein each of R¹¹ and R¹² is benzyl.

In some embodiments is a compound of Formula (I) or (Im) having the structure of Formula (Im1), or a pharmaceutically acceptable salt thereof.

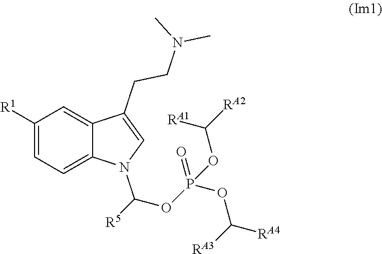

wherein:
R¹ is methoxy or hydrogen;
each of R^{A1}, R^{A3}, and R⁶ is independently hydrogen, alkyl, or cycloalkyl; and
each of R^{A2} and R^{A4} is independently alkyl, heteroalkyl, cycloalkyl, heterocyclylalkyl, aryl, or heteroaryl, —OC(O)R¹⁵, or —OC(O)OR¹⁶,
   wherein each alkyl, heteroalkyl, cycloalkyl, heterocyclylalkyl, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Im1), or a pharmaceutically acceptable salt or solvate thereof, wherein each of R^{A1}, R^{A3}, and R⁵ is independently hydrogen, methyl, ethyl, isopropyl, or tert-butyl. In some embodiments is a compound of Formula (Im1), or a pharmaceutically acceptable salt or solvate thereof, wherein R⁵ is hydrogen. In some embodiments is a compound of Formula (Im1), or a pharmaceutically acceptable salt or solvate thereof, wherein each of R^{A1} and R^{A3} is hydrogen.

In some embodiments is a compound of Formula (Im1), or a pharmaceutically acceptable salt or solvate thereof, wherein each of R^{A2} and R^{A4} is —OC(O)R¹⁵. In some embodiments is a compound of Formula (Im1), or a pharmaceutically acceptable salt or solvate thereof, wherein each of R^{A2} and R^{A4} is —OC(O)R¹⁵; and each of R^{A1}, R^{A3}, and R⁴ is independently hydrogen, methyl, ethyl, isopropyl, or tert-butyl. In some embodiments is a compound of Formula (Im1), or a pharmaceutically acceptable salt or solvate thereof, wherein each R¹⁵ is alkyl, cycloalkyl, aryl, or heteroaryl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is independently hydrogen, methyl, ethyl, isopropyl, or tert-butyl; and each R¹⁵ is alkyl, cycloalkyl, aryl, or heteroaryl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is independently hydrogen, methyl, ethyl, isopropyl, or tert-butyl; and each R¹⁵ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is hydrogen; and each R¹⁵ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is independently hydrogen, methyl, ethyl, isopropyl, or tert-butyl; and each R¹⁵ is phenyl or 4-nitrophenyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is hydrogen; and each R¹⁵ is phenyl or 4-nitrophenyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is independently hydrogen, methyl, ethyl, isopropyl, or tert-butyl; and each R¹⁵ is benzyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is hydrogen; and each R¹⁵ is benzyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is independently hydrogen, methyl, ethyl, isopropyl, or tert-butyl; and each R¹⁵ is 2-pyridyl, 3-pyridyl, 4-pyridyl, 2-pyrimidyl, or 4-pyrimidyl. In some embodiments is a compound of Formula (Im1), or a pharmaceutically acceptable salt or solvate thereof, wherein each of R^{A2} and R^{A4} is —OC(O)R¹⁵; each of R^{A1}, R^{A3}, and R⁵ is hydrogen; and each R¹⁵ is 2-pyridyl, 3-pyridyl, 4-pyridyl, 2-pyrimidyl, or 4-pyrimidyl.

In some embodiments is a compound of Formula (Im1), or a pharmaceutically acceptable salt or solvate thereof, wherein each of R^{A2} and R^{A4} is —OC(O)OR¹⁶. In some embodiments is a compound of Formula (Im1), or a pharmaceutically acceptable salt or solvate thereof, wherein each of R^{A2} and R^{A4} is —OC(O)OR¹⁶; and each of R^{A1}, R^{A3}, and R⁵ is independently hydrogen, methyl, ethyl, isopropyl, or tert-butyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is independently hydrogen, methyl, ethyl, isopropyl, or tert-butyl; and each R¹⁶ is alkyl, cycloalkyl, aryl, or heteroaryl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is independently hydrogen, methyl, ethyl, isopropyl, or tert-butyl; and each R¹⁶ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is hydrogen; and each R¹⁶ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is hydrogen; and each R¹⁶ is isopropyl. In some embodiments is a compound of Formula (Im1), or a pharmaceutically acceptable salt or solvate thereof, wherein each R^{A2} and R^{A4} is —OC(O)OR¹⁶; each of R^{A1}, R^{A3}, and R⁵ is independently hydrogen, methyl, ethyl, isopropyl, or tert-butyl; and each R¹⁶ is phenyl or 4-nitrophenyl. In some embodiments is a compound of Formula (Im1), or a pharmaceutically acceptable salt or solvate thereof, wherein each R^{A2} and R^{A4} is —OC(O)OR¹⁶; each of R^{A1}, R^{A3}, and R⁵ is hydrogen; and each R¹⁶ is phenyl or 4-nitrophenyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is independently hydrogen, methyl, ethyl, isopropyl, or tert-butyl; and each R¹⁶ is benzyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is hydrogen; and each R¹⁶ is benzyl. In some embodiments is a compound of Formula (Im1), or a pharmaceutically acceptable salt or solvate thereof, wherein each of R^{A2} and R^{A4} is —OC(O)OR¹⁶; each of R^{A1}, R^{A3}, and R⁵ is independently hydrogen, methyl, ethyl, isopropyl, or tert-butyl; and each R¹⁶ is 2-pyridyl, 3-pyridyl, 4-pyridyl, 2-pyrimidyl, or 4-pyrimidyl. In some embodiments is a compound of Formula (Im1), or a pharmaceutically acceptable salt or solvate thereof, wherein each of R^{A2} and R^{A4} is —OC(O)OR¹⁶; each R^{A1}, R^{A3}, and R⁵ is hydrogen; and each R¹⁶ is 2-pyridyl, 3-pyridyl, 4-pyridyl, 2-pyrimidyl, or 4-pyrimidyl.

In some embodiments is a compound of Formula (I), (Im), or (Im1), or a pharmaceutically acceptable salt or solvate thereof, wherein the compound is:

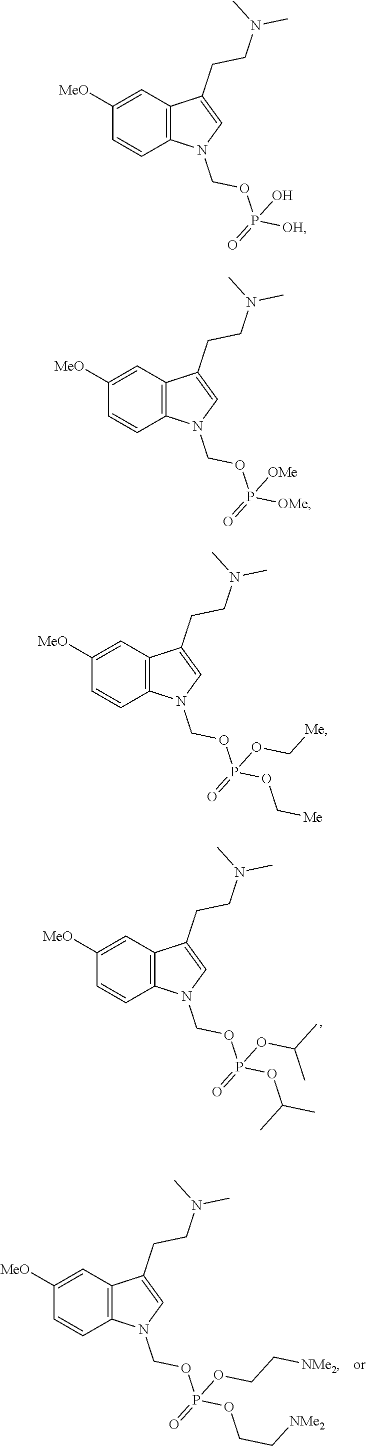

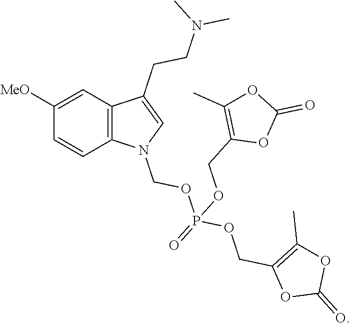 [SMILES: COc1ccc2c(c1)c(CCN(C)C)cn2COP(=O)(OCc1oc(=O)oc1C)OCc1oc(=O)oc1C]

In some embodiments is a compound of Formula (I), (Im), or (Im1), or a pharmaceutically acceptable salt or solvate thereof, wherein the compound is:

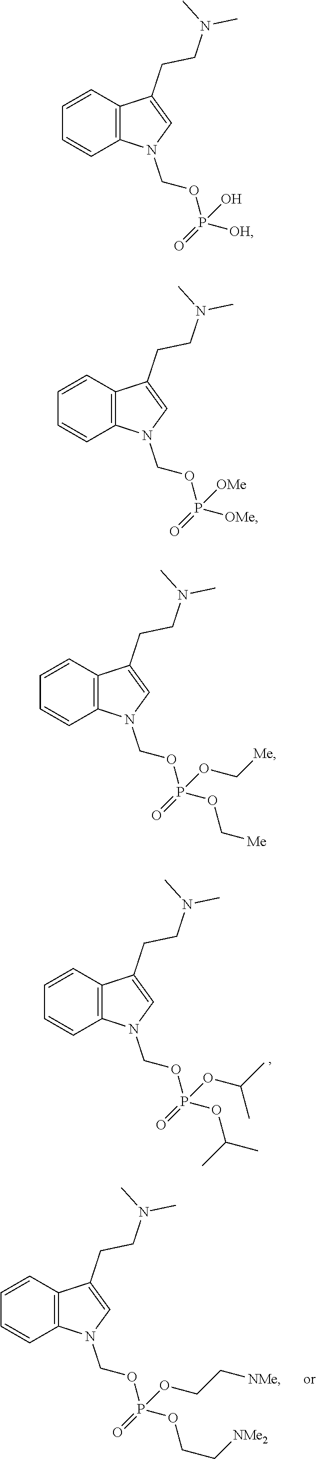

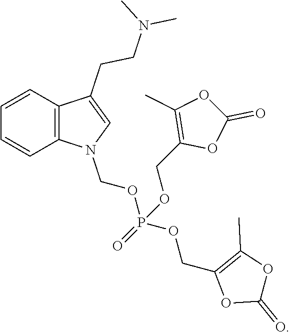 [SMILES: Cc1oc(=O)oc1COP(=O)(OCc1oc(=O)oc1C)OCn1cc(CCN(C)C)c2ccccc21]

In some embodiments is a compound of Formula (I), (Im), or (Im1), or a pharmaceutically acceptable salt or solvate thereof, wherein the compound is:

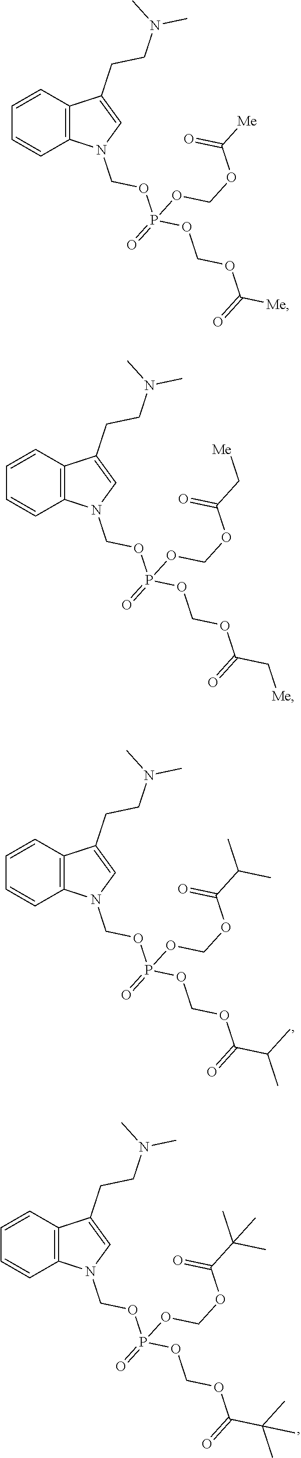 [SMILES: CC(=O)OCOP(=O)(OCOC(C)=O)OCn1cc(CCN(C)C)c2ccccc21 ; CCC(=O)OCOP(=O)(OCOC(=O)CC)OCn1cc(CCN(C)C)c2ccccc21 ; CC(C)C(=O)OCOP(=O)(OCOC(=O)C(C)C)OCn1cc(CCN(C)C)c2ccccc21 ; CN(C)CCc1cn(COP(=O)(OCOC(=O)C(C)(C)C)OCOC(=O)C(C)(C)C)c2ccccc12]

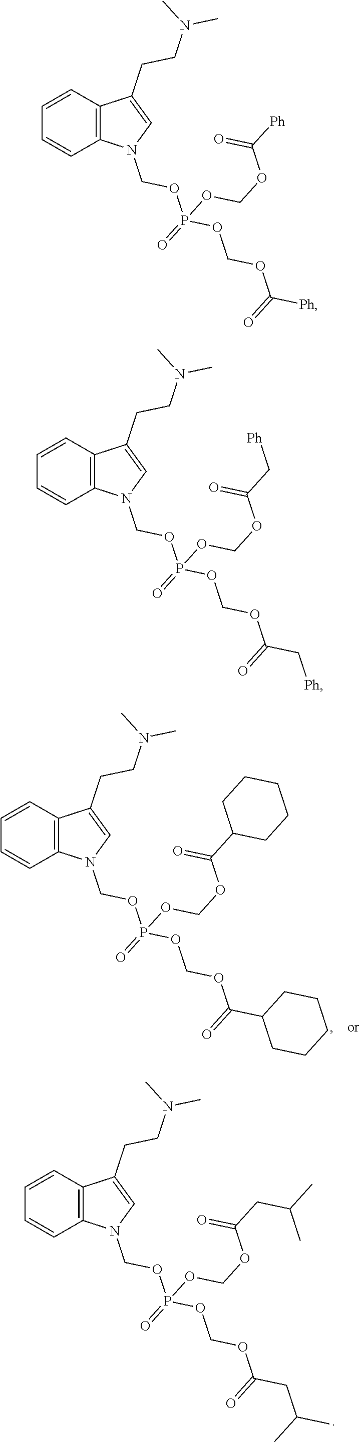 [SMILES: CN(C)CCc1cn(COP(=O)(OCOC(=O)c2ccccc2)OCOC(=O)c2ccccc2)c2ccccc12 ; CN(C)CCc1cn(COP(=O)(OCOC(=O)Cc2ccccc2)OCOC(=O)Cc2ccccc2)c2ccccc12 ; CN(C)CCc1cn(COP(=O)(OCOC(=O)C2CCCCC2)OCOC(=O)C2CCCCC2)c2ccccc12 ; CC(C)CC(=O)OCOP(=O)(OCOC(=O)CC(C)C)OCn1cc(CCN(C)C)c2ccccc21]

In some embodiments is a compound of Formula (I), (Im), or (Im1), or a pharmaceutically acceptable salt or solvate thereof, wherein the compound is:

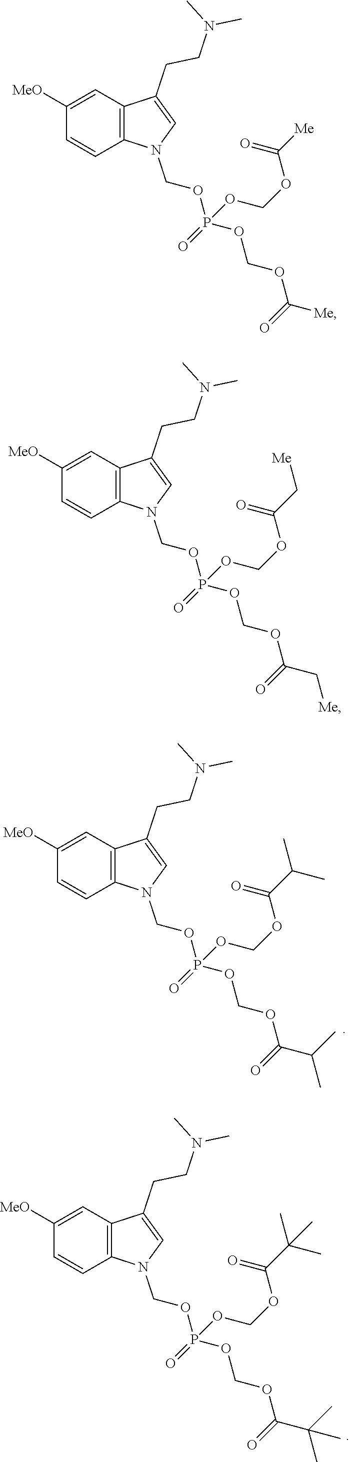 [SMILES: COc1ccc2c(c1)c(CCN(C)C)cn2COP(=O)(OCOC(C)=O)OCOC(C)=O ; CCC(=O)OCOP(=O)(OCOC(=O)CC)OCn1cc(CCN(C)C)c2cc(OC)ccc21 ; COc1ccc2c(c1)c(CCN(C)C)cn2COP(=O)(OCOC(=O)C(C)C)OCOC(=O)C(C)C ; COc1ccc2c(c1)c(CCN(C)C)cn2COP(=O)(OCOC(=O)C(C)(C)C)OCOC(=O)C(C)(C)C]

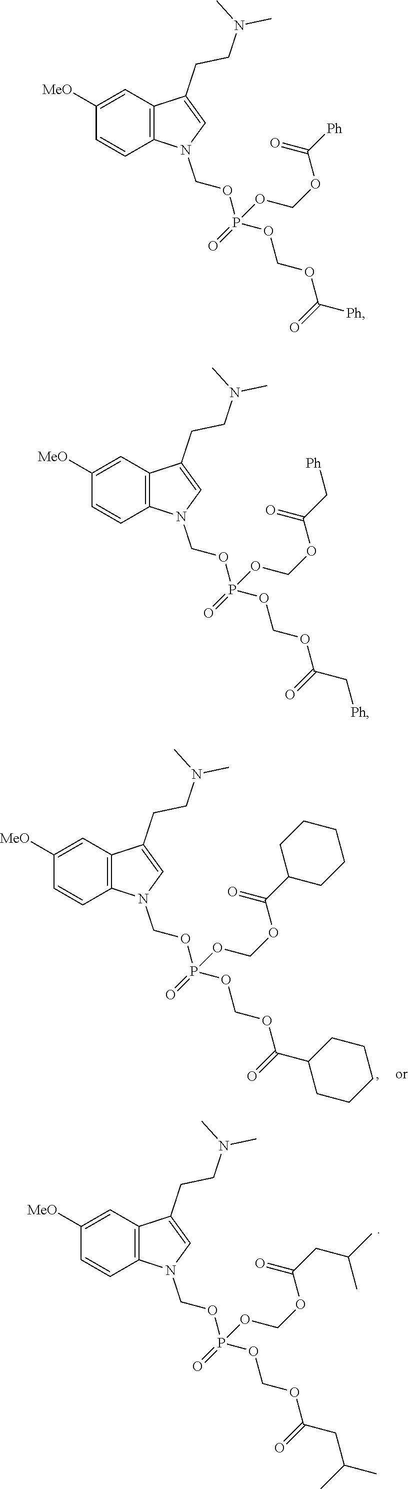 [SMILES: COc1ccc2c(c1)c(CCN(C)C)cn2COP(=O)(OCOC(=O)c1ccccc1)OCOC(=O)c1ccccc1 ; COc1ccc2c(c1)c(CCN(C)C)cn2COP(=O)(OCOC(=O)Cc1ccccc1)OCOC(=O)Cc1ccccc1 ; COc1ccc2c(c1)c(CCN(C)C)cn2COP(=O)(OCOC(=O)C1CCCCC1)OCOC(=O)C1CCCCC1 ; COc1ccc2c(c1)c(CCN(C)C)cn2COP(=O)(OCOC(=O)CC(C)C)OCOC(=O)CC(C)C]

In some embodiments is a compound of Formula (I), (Im), or (Im1), or a pharmaceutically acceptable salt or solvate thereof, wherein the compound is:

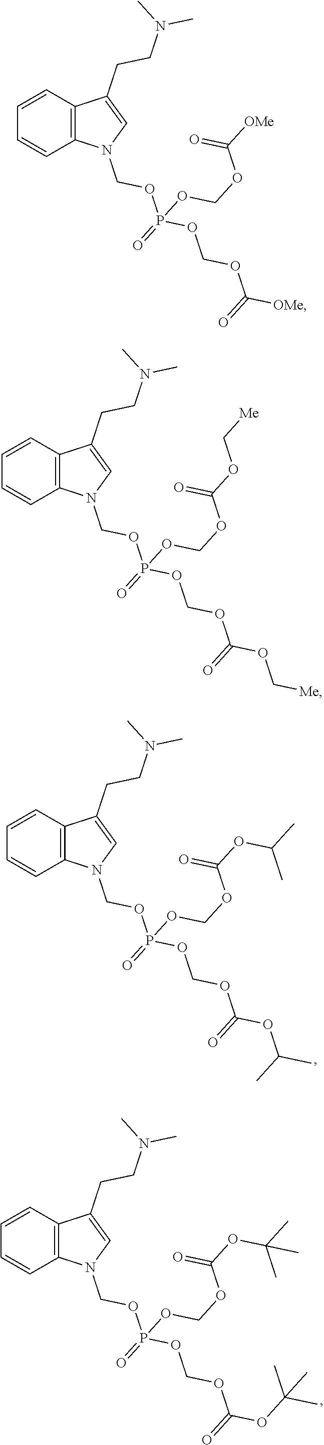 [SMILES: COC(=O)OCOP(=O)(OCOC(=O)OC)OCn1cc(CCN(C)C)c2ccccc21 ; CCOC(=O)OCOP(=O)(OCOC(=O)OCC)OCn1cc(CCN(C)C)c2ccccc21 ; CC(C)OC(=O)OCOP(=O)(OCOC(=O)OC(C)C)OCn1cc(CCN(C)C)c2ccccc21 ; CN(C)CCc1cn(COP(=O)(OCOC(=O)OC(C)(C)C)OCOC(=O)OC(C)(C)C)c2ccccc12]

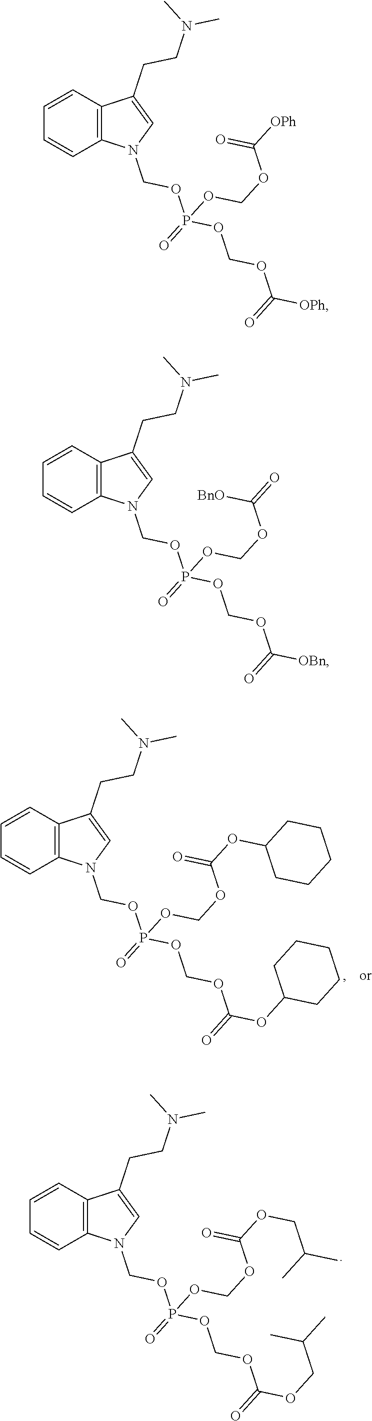 [SMILES: CN(C)CCc1cn(COP(=O)(OCOC(=O)Oc2ccccc2)OCOC(=O)Oc2ccccc2)c2ccccc12 ; CN(C)CCc1cn(COP(=O)(OCOC(=O)OCc2ccccc2)OCOC(=O)OCc2ccccc2)c2ccccc12 ; CN(C)CCc1cn(COP(=O)(OCOC(=O)OC2CCCCC2)OCOC(=O)OC2CCCCC2)c2ccccc12 ; CC(C)COC(=O)OCOP(=O)(OCOC(=O)OCC(C)C)OCn1cc(CCN(C)C)c2ccccc21]

In some embodiments is a compound of Formula (I), (Im), or (Im1), or a pharmaceutically acceptable salt or solvate thereof, wherein the compound is:

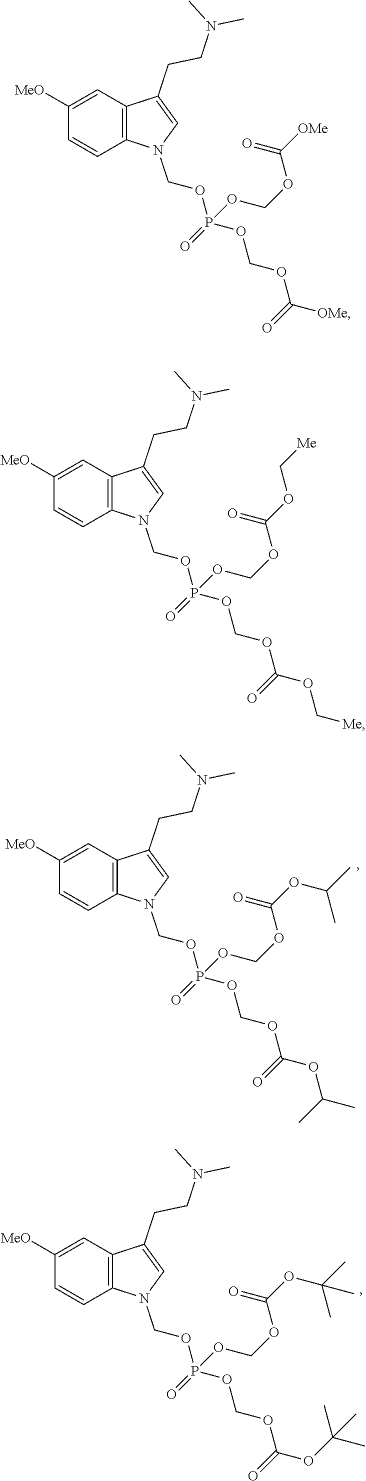 [SMILES: COC(=O)OCOP(=O)(OCOC(=O)OC)OCn1cc(CCN(C)C)c2cc(OC)ccc21 ; CCOC(=O)OCOP(=O)(OCOC(=O)OCC)OCn1cc(CCN(C)C)c2cc(OC)ccc21 ; COc1ccc2c(c1)c(CCN(C)C)cn2COP(=O)(OCOC(=O)OC(C)C)OCOC(=O)OC(C)C ; COc1ccc2c(c1)c(CCN(C)C)cn2COP(=O)(OCOC(=O)OC(C)(C)C)OCOC(=O)OC(C)(C)C]

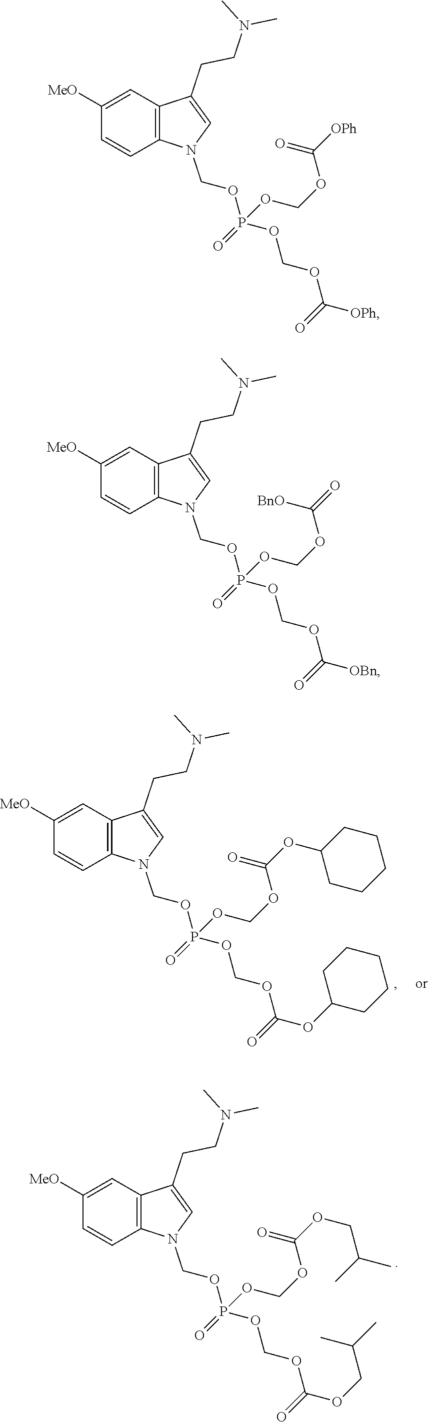 [SMILES: COc1ccc2c(c1)c(CCN(C)C)cn2COP(=O)(OCOC(=O)Oc1ccccc1)OCOC(=O)Oc1ccccc1 ; COc1ccc2c(c1)c(CCN(C)C)cn2COP(=O)(OCOC(=O)OCc1ccccc1)OCOC(=O)OCc1ccccc1 ; COc1ccc2c(c1)c(CCN(C)C)cn2COP(=O)(OCOC(=O)OC1CCCCC1)OCOC(=O)OC1CCCCC1 ; COc1ccc2c(c1)c(CCN(C)C)cn2COP(=O)(OCOC(=O)OCC(C)C)OCOC(=O)OCC(C)C]

In some embodiments is a compound of Formula (I), (Im), or (Im1), or a pharmaceutically acceptable salt or solvate thereof, wherein the compound is:

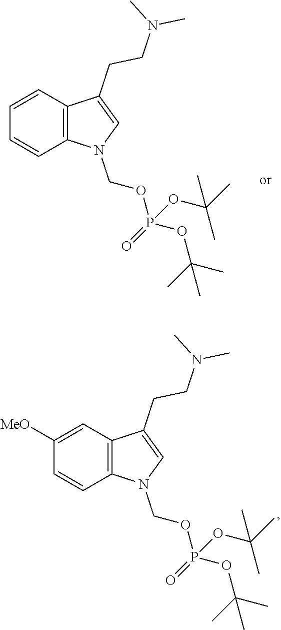 [SMILES: CN(C)CCc1cn(COP(=O)(OC(C)(C)C)OC(C)(C)C)c2ccccc12 ; COc1ccc2c(c1)c(CCN(C)C)cn2COP(=O)(OC(C)(C)C)OC(C)(C)C]

In some embodiments is a compound of Formula (I), (Im), or (Im1) having the structure of Formula (Im1a), or a pharmaceutically acceptable salt thereof:

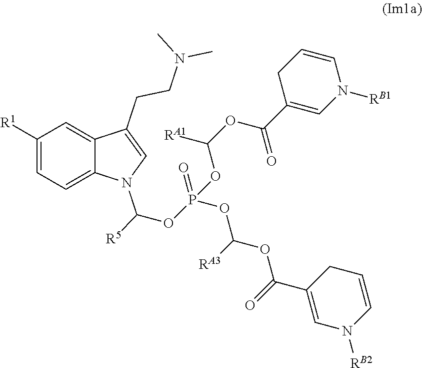

wherein:
R¹ is methoxy or hydrogen;
each of R^{A}, R^{A3}, and R⁵ is independently hydrogen, alkyl, or cycloalkyl, wherein each of alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R^{B1} and R^{B2} is independently hydrogen or alkyl that is unsubstituted or substituted with one or more halogen, amino, cyano, hydroxyl, alkyl, acetyl, or benzoyl.

In some embodiments is a compound of Formula (Im1a), or a pharmaceutically acceptable salt or solvate thereof, wherein each of R^{B1} and R^{B2} is independently hydrogen, methyl, ethyl, n-propyl, isopropyl, n-butyl, tert-butyl, pentan-3-yl, or benzyl. In some embodiments is a compound of Formula (Im1a), or a pharmaceutically acceptable salt or solvate thereof, wherein each of R^{A1}, R^{A3}, and R⁵ is independently hydrogen, methyl, ethyl, isopropyl, or tert-butyl. In some embodiments is a compound of Formula (Im1a), or a pharmaceutically acceptable salt or solvate thereof, wherein each of R^{A1}, R^{A3}, and R⁵ is hydrogen. In some embodiments is a compound of Formula (Im1a), or a pharmaceutically acceptable salt or solvate thereof, wherein each of R^{A1}, R^{A3}, and R⁵ is independently hydrogen, methyl, ethyl, isopropyl, or tert-butyl; and each of R^{B1} and R^{B2} is hydrogen, methyl, ethyl, n-propyl, isopropyl, n-butyl, tert-butyl, pentan-3-yl, or benzyl.

In some embodiments is a compound of Formula (I), (Im), (Im1), or (Im1a), or a pharmaceutically acceptable salt or solvate thereof, wherein the compound is:

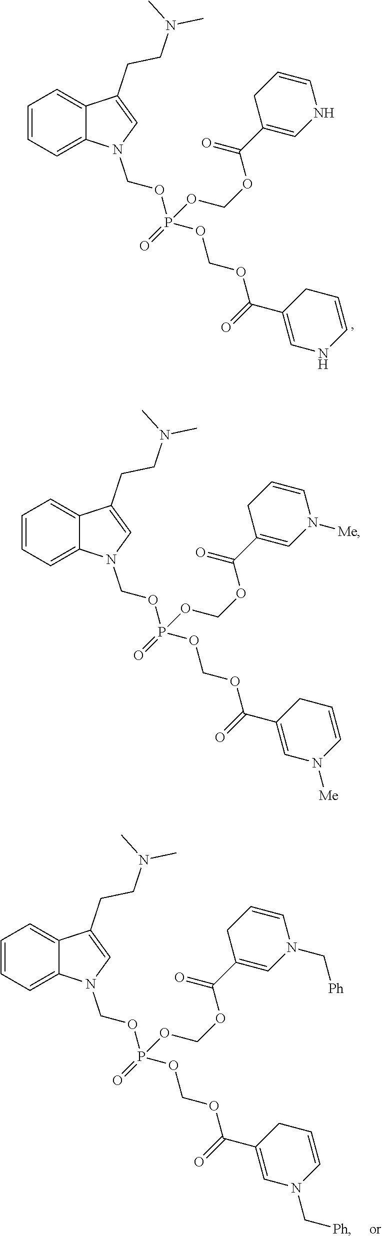 [SMILES: CN(C)CCc1cn(COP(=O)(OCOC(=O)C2=CNC=CC2)OCOC(=O)C2=CNC=CC2)c2ccccc12 ; CN1C=CCC(C(=O)OCOP(=O)(OCOC(=O)C2=CN(C)C=CC2)OCn2cc(CCN(C)C)c3ccccc32)=C1 ; CN(C)CCc1cn(COP(=O)(OCOC(=O)C2=CN(Cc3ccccc3)C=CC2)OCOC(=O)C2=CN(Cc3ccccc3)C=CC2)c2ccccc12]

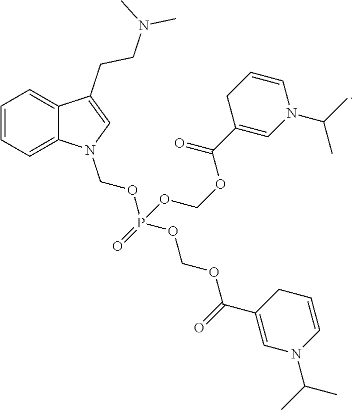 [SMILES: CC(C)N1C=CCC(C(=O)OCOP(=O)(OCOC(=O)C2=CN(C(C)C)C=CC2)OCn2cc(CCN(C)C)c3ccccc32)=C1]

In some embodiments is a compound of Formula (I), (Im), (Im1), or (Im1a), or a pharmaceutically acceptable salt or solvate thereof, wherein the compound is:

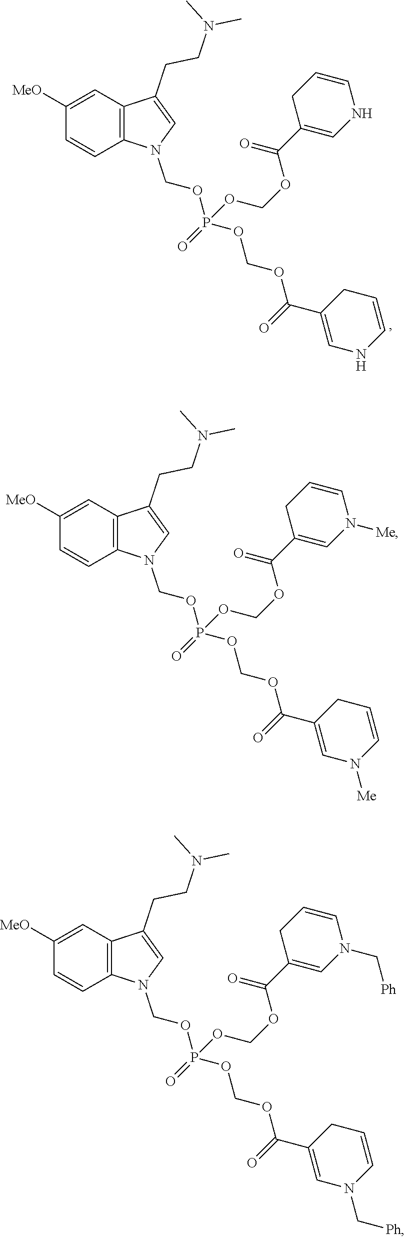 [SMILES: COc1ccc2c(c1)c(CCN(C)C)cn2COP(=O)(OCOC(=O)C1=CNC=CC1)OCOC(=O)C1=CNC=CC1 ; COc1ccc2c(c1)c(CCN(C)C)cn2COP(=O)(OCOC(=O)C1=CN(C)C=CC1)OCOC(=O)C1=CN(C)C=CC1 ; COc1ccc2c(c1)c(CCN(C)C)cn2COP(=O)(OCOC(=O)C1=CN(Cc2ccccc2)C=CC1)OCOC(=O)C1=CN(Cc2ccccc2)C=CC1]

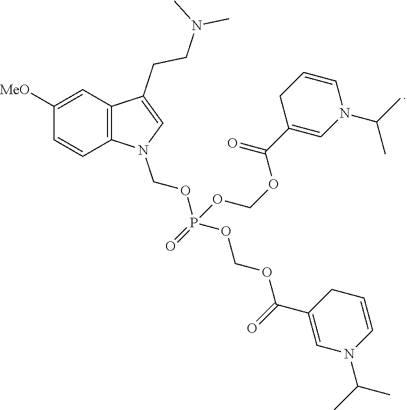 [SMILES: COc1ccc2c(c1)c(CCN(C)C)cn2COP(=O)(OCOC(=O)C1=CN(C(C)C)C=CC1)OCOC(=O)C1=CN(C(C)C)C=CC1]

In some embodiments is a compound of Formula (I) having the structure of Formula (In), or a pharmaceutically acceptable salt thereof:

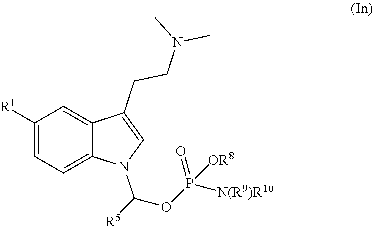

wherein:
R¹ is methoxy or hydrogen;
R⁵ is hydrogen, alkyl, or cycloalkyl;
R⁸ is hydrogen, alkyl, cycloalkyl, aryl, heterocyclylalkyl, or heteroaryl; and
each of R⁹ and R¹⁰ is independently hydrogen or alkyl,
   wherein each cycloalkyl, aryl, heterocyclylalkyl, and heteroaryl is independently unsubstituted or substituted with one or more R^{A}.

In some embodiments is a compound of Formula (I) or (In), or a pharmaceutically acceptable salt or solvate thereof, wherein R⁵ is alkyl. In some embodiments is a compound of Formula (I) or (In), or a pharmaceutically acceptable salt or solvate thereof, wherein R⁵ is hydrogen. In some embodiments is a compound of Formula (I) or (In), or a pharmaceutically acceptable salt or solvate thereof, wherein R⁵ is hydrogen or unsubstituted alkyl. In some embodiments is a compound of Formula (I) or (In), or a pharmaceutically acceptable salt or solvate thereof, wherein R⁵ is hydrogen, methyl, ethyl, or tert-butyl. In some embodiments is a compound of Formula (I) or (In), or a pharmaceutically acceptable salt or solvate thereof, wherein R⁸ is alkyl, cycloalkyl, aryl, heterocyclylalkyl, or heteroaryl. In some embodiments is a compound of Formula (I) or (In), or a pharmaceutically acceptable salt or solvate thereof, wherein R⁸ is hydrogen. In some embodiments is a compound of Formula (I) or (In), or a pharmaceutically acceptable salt or solvate thereof, wherein R⁸ is alkyl or cycloalkyl. In some embodiments is a compound of Formula (I) or (In), or a pharmaceutically acceptable salt or solvate thereof, wherein R⁸ is unsubstituted alkyl or unsubstituted cycloalkyl. In some embodiments is a compound of Formula (I) or (In), or a pharmaceutically acceptable salt or solvate thereof, wherein R⁸ is methyl, ethyl, n-propyl, n-butyl, n-pentyl, n-hexyl, isopropyl, tert-butyl, cyclopropyl, cyclobutyl, cyclopentyl, cyclohexyl, or cycloheptyl. In some embodiments is a compound of Formula (I) or (In), or a pharmaceutically acceptable salt or solvate thereof, wherein R⁸ is phenyl. In some embodiments is a compound of Formula (I) or (In), or a pharmaceutically acceptable salt or solvate thereof, wherein R⁸ is 4-nitrophenyl. In some embodiments is a compound of Formula (I) or (In), or a pharmaceutically acceptable salt or solvate thereof, wherein R⁸ is benzyl. In some embodiments is a compound of Formula (I) or (In), or a pharmaceutically acceptable salt or solvate thereof, wherein R⁸ is 2-pyridyl, 3-pyridyl, 4-pyridyl, 2-pyrimidyl, or 4-pyrimidyl. In some embodiments is a compound of Formula (I) or (In), or a pharmaceutically acceptable salt or solvate thereof, wherein R⁹ is hydrogen. In some embodiments is a compound of Formula (I) or (In), or a pharmaceutically acceptable salt or solvate thereof, wherein R⁹ is hydrogen, and R¹⁰ is alkyl.

In some embodiments is a compound of Formula (I) or (In) having the structure of Formula (In1), or a pharmaceutically acceptable salt thereof:

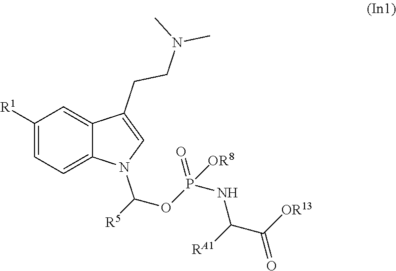

wherein:
R¹ is methoxy or hydrogen;
R^{A1} is hydrogen, alkyl, or cycloalkyl, wherein each of alkyl and cycloalkyl is unsubstituted or substituted with alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R⁵ and R⁸ is hydrogen, alkyl, cycloalkyl, aryl, heterocyclylalkyl, or heteroaryl,
   wherein alkyl, cycloalkyl, aryl, heterocyclylalkyl, and heteroaryl is independently unsubstituted or substituted with one or more R^{A}; and
R¹³ is hydrogen or alkyl that is unsubstituted or substituted with one or more R^{B}.

In some embodiments is a compound of Formula (In1), or a pharmaceutically acceptable salt or solvate thereof, wherein each of R⁵ and R^{A1} is hydrogen or alkyl. In some embodiments is a compound of Formula (In1), or a pharmaceutically acceptable salt or solvate thereof, wherein each of R⁵ and R^{A1} is hydrogen or unsubstituted alkyl. In some embodiments is a compound of Formula (In1), or a pharmaceutically acceptable salt or solvate thereof, wherein each of R⁵ and R^{A1} is hydrogen, methyl, ethyl, or tert-butyl. In some embodiments is a compound of Formula (In1), or a pharmaceutically acceptable salt or solvate thereof, wherein each of R⁵ and R^{A1} is hydrogen. In some embodiments is a compound of Formula (In1), or a pharmaceutically acceptable salt or solvate thereof, wherein R⁸ is alkyl or cycloalkyl. In some embodiments is a compound of Formula (In1), or a pharmaceutically acceptable salt or solvate thereof, wherein R⁸ is unsubstituted alkyl or unsubstituted cycloalkyl. In some embodiments is a compound of Formula (In1), or a pharmaceutically acceptable salt or solvate thereof, wherein R⁸ is methyl, ethyl, n-propyl, n-butyl, n-pentyl, n-hexyl, isopropyl, tert-butyl, cyclopropyl, cyclobutyl, cyclopentyl, cyclohexyl, or cycloheptyl. In some embodiments is a compound of Formula (In1), or a pharmaceutically acceptable salt or solvate thereof, wherein R⁸ is phenyl. In some embodiments is a compound of Formula (In1), or a pharmaceutically acceptable salt or solvate thereof, wherein R⁸ is 4-nitrophenyl. In some embodiments is a compound of Formula (In1), or a pharmaceutically acceptable salt or solvate thereof, wherein R⁸ is benzyl. In some embodiments is a compound of Formula (In1), or a pharmaceutically acceptable salt or solvate thereof, wherein R⁸ is 2-pyridyl, 3-pyridyl, 4-pyridyl, 2-pyrimidyl, or 4-pyrimidyl. In some embodiments is a compound of Formula (In1), or a pharmaceutically acceptable salt or solvate thereof, wherein R¹³ is alkyl. In some embodiments is a compound of Formula (In1), or a pharmaceutically acceptable salt or solvate thereof, wherein R¹³ is unsubstituted alkyl. In some embodiments is a compound of Formula (In1), or a pharmaceutically acceptable salt or solvate thereof, wherein R¹³ is methyl, ethyl, n-propyl, isopropyl, n-butyl, tert-butyl, or —CH₂CH(Et)₂. In some embodiments is a compound of Formula (In1), or a pharmaceutically acceptable salt or solvate thereof, wherein each of R⁵ and R^{A1} is hydrogen or alkyl; and R¹³ is methyl, ethyl, n-propyl, isopropyl, n-butyl, tert-butyl, or —CH₂CH(Et)₂. In some embodiments is a compound of Formula (In1), or a pharmaceutically acceptable salt or solvate thereof, wherein each of R⁵ and R^{A1} is hydrogen or unsubstituted alkyl; and R¹³ is methyl, ethyl, n-propyl, isopropyl, n-butyl, tert-butyl, or —CH₂CH(Et)₂. In some embodiments is a compound of Formula (In1), or a pharmaceutically acceptable salt or solvate thereof, wherein each of R⁵ and R^{A1} is hydrogen; and R¹³ is methyl, ethyl, n-propyl, isopropyl, n-butyl, tert-butyl, or —CH₂CH(Et)₂.

In some embodiments is a compound of Formula (I), (In), or (In1), or a pharmaceutically acceptable salt or solvate thereof, wherein the compound is:

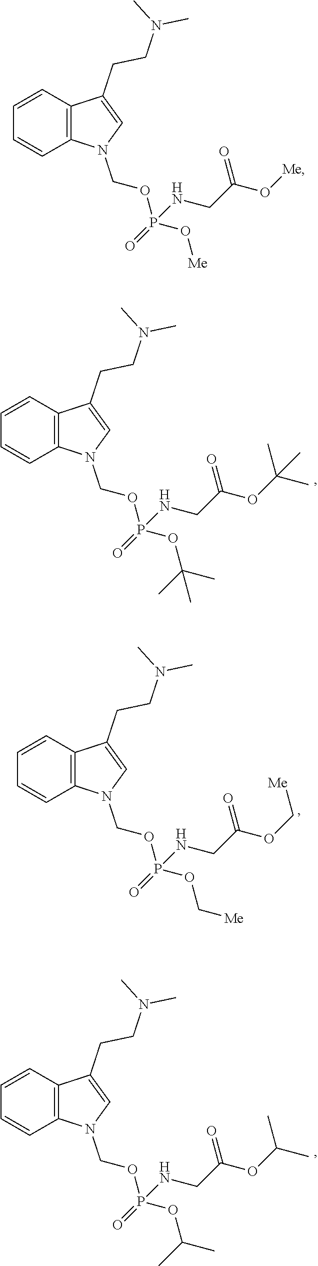 [SMILES: COC(=O)CNP(=O)(OC)OCn1cc(CCN(C)C)c2ccccc21 ; CN(C)CCc1cn(COP(=O)(NCC(=O)OC(C)(C)C)OC(C)(C)C)c2ccccc12 ; CCOC(=O)CNP(=O)(OCC)OCn1cc(CCN(C)C)c2ccccc21 ; CC(C)OC(=O)CNP(=O)(OCn1cc(CCN(C)C)c2ccccc21)OC(C)C]

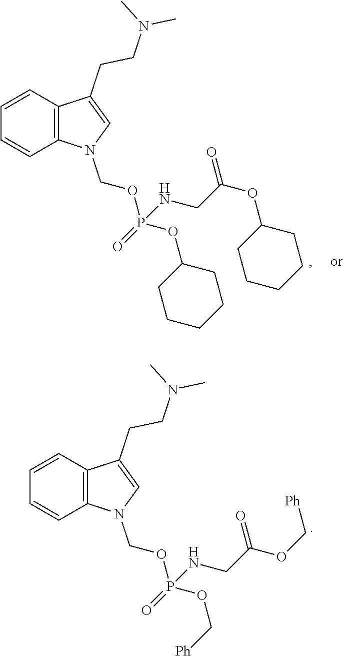 [SMILES: CN(C)CCc1cn(COP(=O)(NCC(=O)OC2CCCCC2)OC2CCCCC2)c2ccccc12 ; CN(C)CCc1cn(COP(=O)(NCC(=O)OCc2ccccc2)OCc2ccccc2)c2ccccc12]

In some embodiments is a compound of Formula (I), (In), or (In1), or a pharmaceutically acceptable salt or solvate thereof, wherein the compound is:

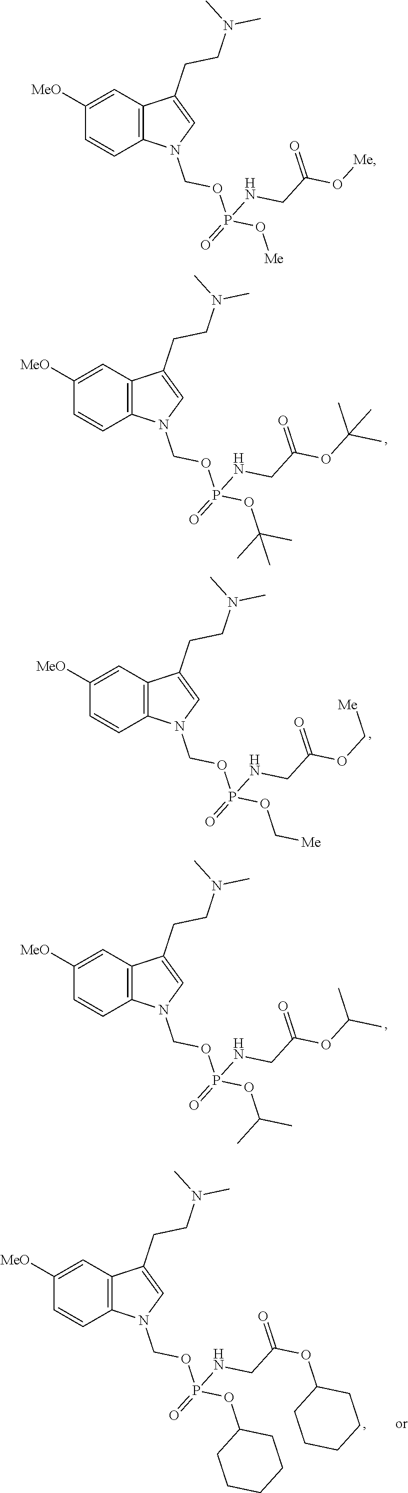

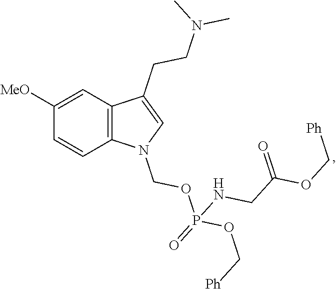 [SMILES: COc1ccc2c(c1)c(CCN(C)C)cn2COP(=O)(NCC(=O)OCc1ccccc1)OCc1ccccc1]

In some embodiments is a compound of Formula (I) having the structure of Formula (Io), or a pharmaceutically acceptable salt thereof:

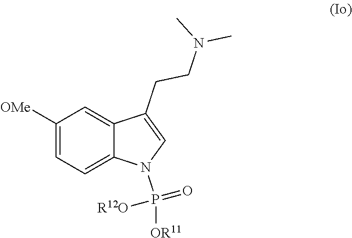

wherein:
R¹ is methoxy or hydrogen; and
each of R¹¹ and R¹² is independently selected from hydrogen, cycloalkyl, aryl, heteroaryl, or alkyl, wherein each cycloalkyl, aryl, heteroaryl, and alkyl is independently unsubstituted or substituted with one or more R^{A}, or R¹¹ and R¹² together with the atoms to which they are attached form a heterocyclylalkyl ring that is unsubstituted or substituted with one or more R^{A}.

In some embodiments is a compound of Formula (I) or (Io), or a pharmaceutically acceptable salt or solvate thereof, wherein each of R¹¹ and R¹² is independently selected from cycloalkyl, aryl, heteroaryl, or alkyl; or R¹¹ and R¹² together with the atom to which they are attached form a heterocyclylalkyl ring. In some embodiments is a compound of Formula (I) or (Io), or a pharmaceutically acceptable salt or solvate thereof, wherein each of R¹¹ and R¹² is independently selected from unsubstituted alkyl, unsubstituted cycloalkyl, unsubstituted aryl, unsubstituted heteroaryl, unsubstituted alkyl, or alkyl substituted with aryl or heteroaryl. In some embodiments is a compound of Formula (I) or (Io), or a pharmaceutically acceptable salt or solvate thereof, wherein R¹¹ is hydrogen, and R¹² is alkyl, cycloalkyl, aryl, heteroaryl, or alkyl. In some embodiments is a compound of Formula (I) or (Io), or a pharmaceutically acceptable salt or solvate thereof, wherein each of R¹¹ and R¹² is alkyl. In some embodiments is a compound of Formula (I) or (Io), or a pharmaceutically acceptable salt or solvate thereof, wherein each of R¹¹ and R¹² is alkyl. In some embodiments is a compound of Formula (I) or (Io), or a pharmaceutically acceptable salt or solvate thereof, wherein each of R¹¹ and R¹² is unsubstituted alkyl. In some embodiments is a compound of Formula (I) or (Io), or a pharmaceutically acceptable salt or solvate thereof, wherein each of R¹¹ and R¹² is alkyl substituted with —OC(O)R¹⁵. In some embodiments is a compound of Formula (I) or (Io), or a pharmaceutically acceptable salt or solvate thereof, wherein each of R¹¹ and R¹² is alkyl substituted with —OC(O)R¹⁵, wherein each R¹⁵ is alkyl, cycloalkyl, heterocyclylalkyl, aryl, or heteroaryl. In some embodiments is a compound of Formula (I) or (Io), or a pharmaceutically acceptable salt or solvate thereof, wherein each of R¹¹ and R¹² is alkyl substituted with —OC(O)R¹⁵, wherein each R¹⁵ is unsubstituted alkyl, unsubstituted cycloalkyl, unsubstituted heterocyclylalkyl, unsubstituted aryl, or unsubstituted heteroaryl. In some embodiments is a compound of Formula (I) or (Io), or a pharmaceutically acceptable salt or solvate thereof, wherein each of R¹¹ and R¹² is alkyl substituted with —OC(O)R¹⁵, wherein each R¹⁵ is heterocyclylalkyl substituted with alkyl or arylalkyl.

In some embodiments is a compound of Formula (I) or (Io), or a pharmaceutically acceptable salt or solvate thereof, wherein each of R¹¹ and R¹² is alkyl, heterocyclylalkyl, or cycloalkyl. In some embodiments is a compound of Formula (I) or (Io), or a pharmaceutically acceptable salt or solvate thereof, wherein each of R¹¹ and R¹² is alkyl. In some embodiments is a compound of Formula (I) or (Io), or a pharmaceutically acceptable salt or solvate thereof, wherein each of R¹¹ and R¹² is unsubstituted alkyl. In some embodiments is a compound of Formula (I) or (Io), or a pharmaceutically acceptable salt or solvate thereof, wherein R¹ is methoxy, and each of R¹¹ and R¹² is unsubstituted alkyl. In some embodiments is a compound of Formula (I) or (Io), or a pharmaceutically acceptable salt or solvate thereof, wherein R¹ is hydrogen, and each of R¹¹ and R¹² is unsubstituted alkyl. In some embodiments is a compound of Formula (I) or (Io), or a pharmaceutically acceptable salt or solvate thereof, wherein R¹ is methoxy, and each of R¹¹ and R¹² is tert-butyl. In some embodiments is a compound of Formula (I) or (Io), or a pharmaceutically acceptable salt or solvate thereof, wherein R¹ is hydrogen, and each of R¹¹ and R¹² is tert-butyl.

In some embodiments is a compound of Formula (I) or (Io), or a pharmaceutically acceptable salt or solvate thereof, wherein each of R¹¹ and R¹² is

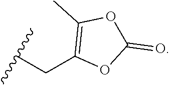 [SMILES: *Cc1oc(=O)oc1C]

In some embodiments is a compound of Formula (I) or (Io), or a pharmaceutically acceptable salt or solvate thereof, wherein each of R¹¹ and R¹² is methyl, ethyl, n-propyl, isopropyl, n-butyl, tert-butyl, n-pentyl, n-hexyl, or 3-methyl-1-butyl. In some embodiments is a compound of Formula (I) or (Io), or a pharmaceutically acceptable salt or solvate thereof, wherein each of R¹¹ and R¹² is cyclopropyl, cyclobutyl, cyclopentyl, cyclohexyl, or cycloheptyl. In some embodiments is a compound of Formula (I) or (Io), or a pharmaceutically acceptable salt or solvate thereof, wherein each of R¹¹ and R¹² is phenyl. In some embodiments is a compound of Formula (I) or (Io), or a pharmaceutically acceptable salt or solvate thereof, wherein each of R¹¹ and R¹² is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (Io), or a pharmaceutically acceptable salt or solvate thereof, wherein each of R¹¹ and R¹² is 4-nitrophenyl. In some embodiments is a compound of Formula (I) or (Io), or a pharmaceutically acceptable salt or solvate thereof, wherein each of R¹¹ and R¹² is benzyl.

In some embodiments is a compound of Formula (I) or (Io) having the structure of Formula (Io1), or a pharmaceutically acceptable salt thereof:

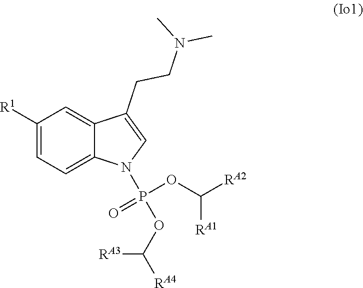

wherein:
R¹ is methoxy or hydrogen;
each of R^{A1} and R^{A3} is independently hydrogen, alkyl, or cycloalkyl; and
each of R^{A2} and R^{A4} is independently alkyl, heteroalkyl, or cycloalkyl, heterocyclylalkyl, aryl, heteroaryl, —OC(O)R¹⁵, or —OC(O)OR¹⁶,
   wherein each alkyl, heteroalkyl, cycloalkyl, heterocyclylalkyl, aryl, and heteroaryl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Io1), or a pharmaceutically acceptable salt or solvate thereof, wherein each of R^{A1} and R^{A3} is independently hydrogen, methyl, ethyl, isopropyl, or tert-butyl. In some embodiments is a compound of Formula (Io1), or a pharmaceutically acceptable salt or solvate thereof, wherein each of R^{A1} and R^{A3} is hydrogen.

In some embodiments is a compound of Formula (Io1), or a pharmaceutically acceptable salt or solvate thereof, wherein each of R^{A2} and R^{A4} is —OC(O)R¹⁵. In some embodiments is a compound of Formula (Io1), or a pharmaceutically acceptable salt or solvate thereof, wherein each of R^{A2} and R^{A4} is —OC(O)R¹⁵; and each of R^{A1} and R^{A3} is independently hydrogen, methyl, ethyl, isopropyl, or tert-butyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is independently hydrogen, methyl, ethyl, isopropyl, or tert-butyl; and each R¹⁵ is alkyl, cycloalkyl, aryl, or heteroaryl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is independently hydrogen, methyl, ethyl, isopropyl, or tert-butyl; and each R¹⁵ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is hydrogen; and each R¹⁵ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is independently hydrogen, methyl, ethyl, isopropyl, or tert-butyl; and each R¹⁵ is phenyl or 4-nitrophenyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is hydrogen; and each R¹⁵ is phenyl or 4-nitrophenyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is independently hydrogen, methyl, ethyl, isopropyl, or tert-butyl; and each R¹⁵ is benzyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is hydrogen; and each R¹⁵ is benzyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is independently hydrogen, methyl, ethyl, isopropyl, or tert-butyl; and each R¹⁵ is 2-pyridyl, 3-pyridyl, 4-pyridyl, 2-pyrimidyl, or 4-pyrimidyl. In some embodiments is a compound of Formula (Io1), or a pharmaceutically acceptable salt or solvate thereof, wherein each of R^{A2} and R^{A4} is —OC(O)R¹⁵; each of R^{A1} and R^{A3} is hydrogen; and each R¹⁵ is 2-pyridyl, 3-pyridyl, 4-pyridyl, 2-pyrimidyl, or 4-pyrimidyl.

In some embodiments is a compound of Formula (Io1), or a pharmaceutically acceptable salt or solvate thereof, wherein each of R^{A2} and R^{A4} is —OC(O)OR¹⁶. In some embodiments is a compound of Formula (Io1), or a pharmaceutically acceptable salt or solvate thereof, wherein each of R^{A2} and R^{A4} is —OC(O)OR¹⁶; and each of R^{A1} and R^{A3} is independently hydrogen, methyl, ethyl, isopropyl, or tert-butyl. In some embodiments is a compound of Formula (Io1), or a pharmaceutically acceptable salt or solvate thereof, wherein each of R^{A2} and R^{A4} is —OC(O)OR¹⁶; each of R^{A1} and R^{A3} is independently hydrogen, methyl, ethyl, isopropyl, or tert-butyl; and each R¹⁶ is alkyl, cycloalkyl, aryl, or heteroaryl. In some embodiments is a compound of Formula (Io1), or a pharmaceutically acceptable salt or solvate thereof, wherein each of R^{A2} and R^{A4} is —OC(O)OR¹⁶; each of R^{A1} and R^{A3} is independently hydrogen, methyl, ethyl, isopropyl, or tert-butyl; and each R¹⁶ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Io1), or a pharmaceutically acceptable salt or solvate thereof, wherein each of R^{A2} and R^{A4} is —OC(O)OR¹⁶; each of R^{A1} and R^{A3} is hydrogen; and each R¹⁶ is methyl, ethyl, n-propyl, n-butyl, n-pentyl, n-hexyl, isopropyl, tert-butyl, 3-methyl-1-butyl, cyclopropyl, or cyclobutyl. In some embodiments is a compound of Formula (Io1), or a pharmaceutically acceptable salt or solvate thereof, wherein each R^{A2} and R^{A4} is —OC(O)OR¹⁶; each of R^{A1} and R^{A3} is independently hydrogen, methyl, ethyl, isopropyl, or tert-butyl; and each R¹⁶ is phenyl or 4-nitrophenyl. In some embodiments is a compound of Formula (Io1), or a pharmaceutically acceptable salt or solvate thereof, wherein each R^{A2} and R^{A4} is —OC(O)OR¹⁶; each of R^{A1} and R^{A3} is hydrogen; and each R¹⁶ is phenyl or 4-nitrophenyl. In some embodiments is a compound of Formula (Io1), or a pharmaceutically acceptable salt or solvate thereof, wherein each of R^{A2} and R^{A4} is —OC(O)OR¹⁶; each R^{A1} and R^{A3} is independently hydrogen, methyl, ethyl, isopropyl, or tert-butyl; and each R¹⁶ is benzyl. In some embodiments is a compound of Formula (Io1), or a pharmaceutically acceptable salt or solvate thereof, wherein each of R^{A2} and R^{A4} is —OC(O)OR¹⁶; each of R^{A1} and R^{A3} is hydrogen; and each R¹⁶ is benzyl. In some embodiments is a compound of Formula (Io1), or a pharmaceutically acceptable salt or solvate thereof, wherein each of R^{A2} and R^{A4} is —OC(O)OR¹⁶; each of R^{A1} and R^{A3} is independently hydrogen, methyl, ethyl, isopropyl, or tert-butyl; and each R¹⁶ is 2-pyridyl, 3-pyridyl, 4-pyridyl, 2-pyrimidyl, or 4-pyrimidyl. In some embodiments is a compound of Formula (Io1), or a pharmaceutically acceptable salt or solvate thereof, wherein each of R^{A2} and R^{A4} is —OC(O)OR¹⁶; each R^{A1} and R^{A3} is hydrogen; and each R¹⁶ is 2-pyridyl, 3-pyridyl, 4-pyridyl, 2-pyrimidyl, or 4-pyrimidyl.

In some embodiments is a compound of Formula (I), (Io), or (Io1), or a pharmaceutically acceptable salt or solvate thereof, wherein the compound is:

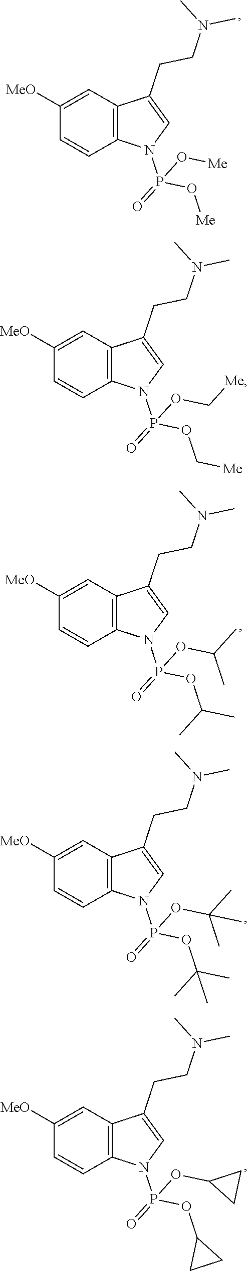

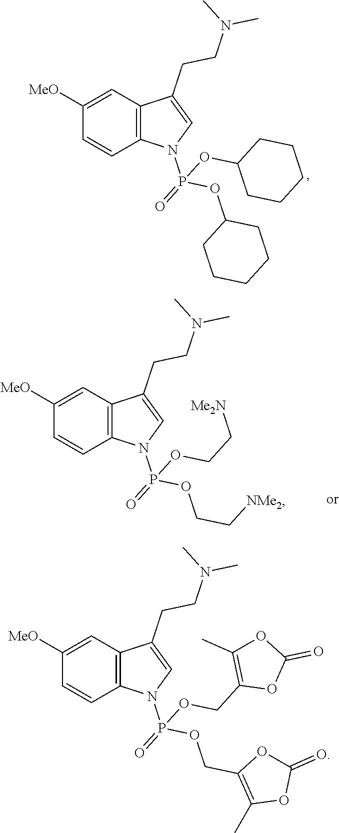

In some embodiments is a compound of Formula (I), (Io), or (Io1), or a pharmaceutically acceptable salt or solvate thereof, wherein:

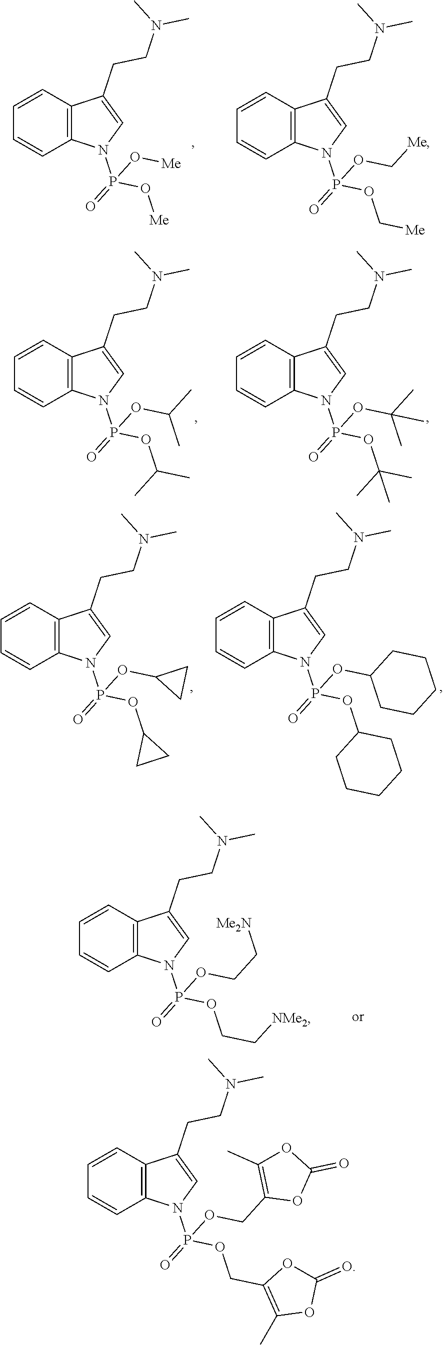

In some embodiments is a compound of Formula (I) or (Io) having the structure of Formula (Io2), or a pharmaceutically acceptable salt thereof:

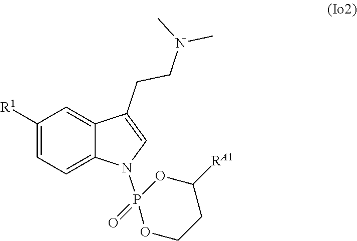

wherein R¹ is methoxy or hydrogen; and R^{A1} is aryl or heteroaryl, each of which is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments is a compound of Formula (Io2), or a pharmaceutically acceptable salt or solvate thereof, wherein R^{A1} is aryl. In some embodiments is a compound of Formula (Io2), or a pharmaceutically acceptable salt or solvate thereof, wherein R^{A1} is aryl substituted with halogen. In some embodiments is a compound of Formula (Io2), or a pharmaceutically acceptable salt or solvate thereof, wherein R^{A1} is

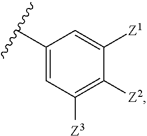

wherein each of Z¹, Z², and Z³ is independently hydrogen or halogen. In some embodiments is a compound of Formula (Io2), or a pharmaceutically acceptable salt or solvate thereof, wherein R^{A1} is

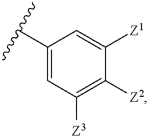

wherein each of Z¹, Z², and Z³ is independently hydrogen, fluoro, chloro, bromo, or iodo. In some embodiments is a compound of Formula (Io2), or a pharmaceutically acceptable salt or solvate thereof, wherein R^{A1} is

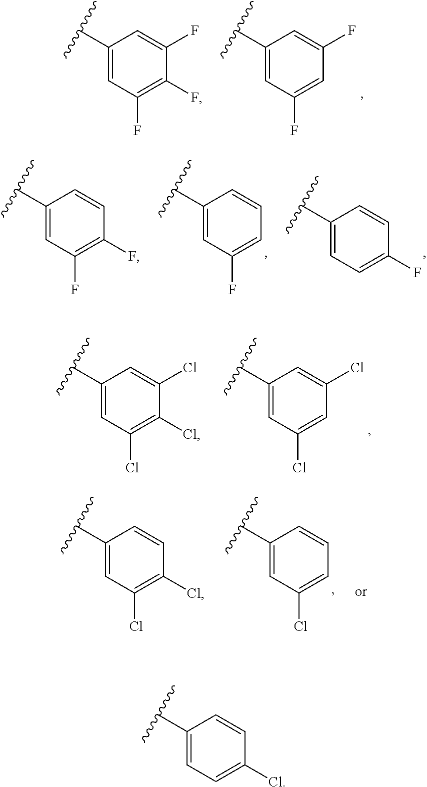

In some embodiments is a compound of Formula (Io2), or a pharmaceutically acceptable salt or solvate thereof, wherein the compound is:

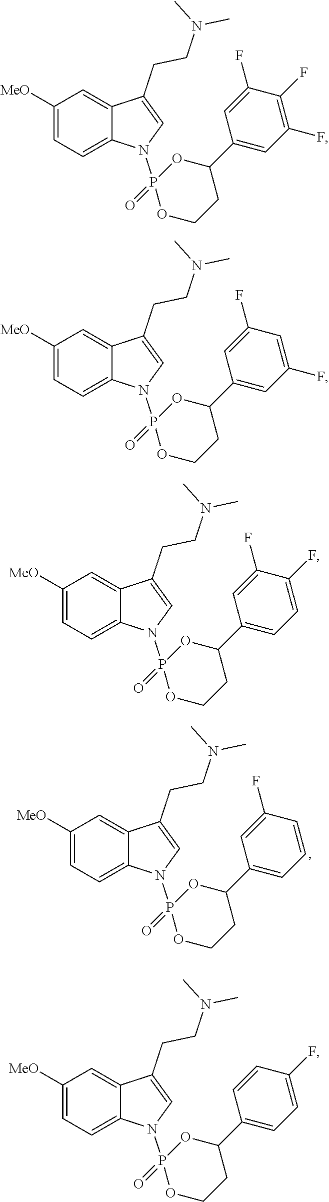

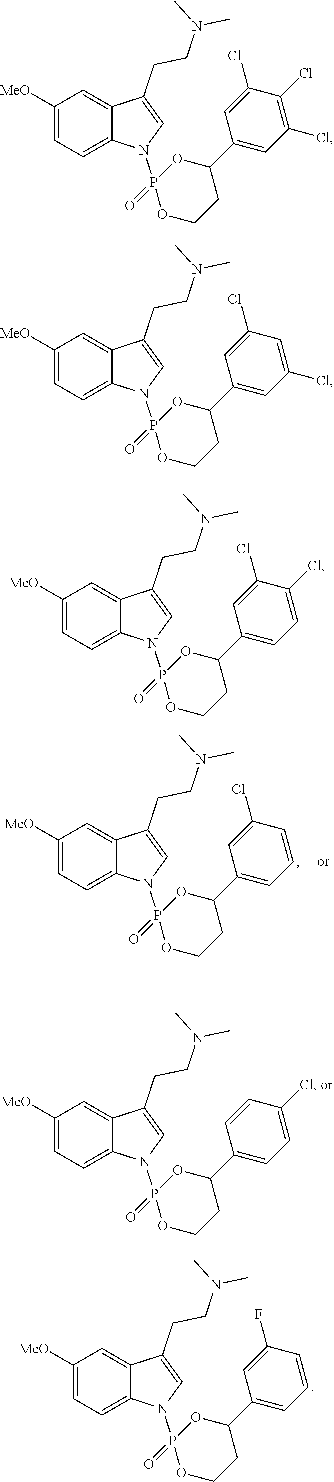

In some embodiments is a compound of Formula (Io2), or a pharmaceutically

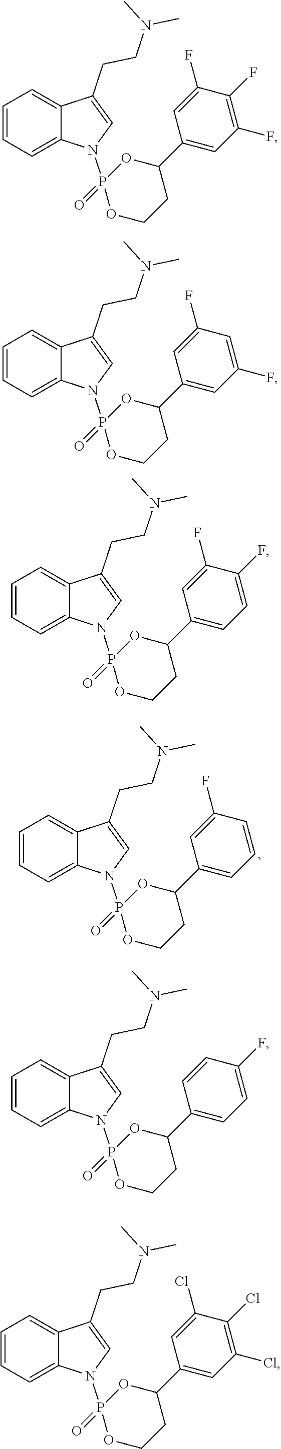

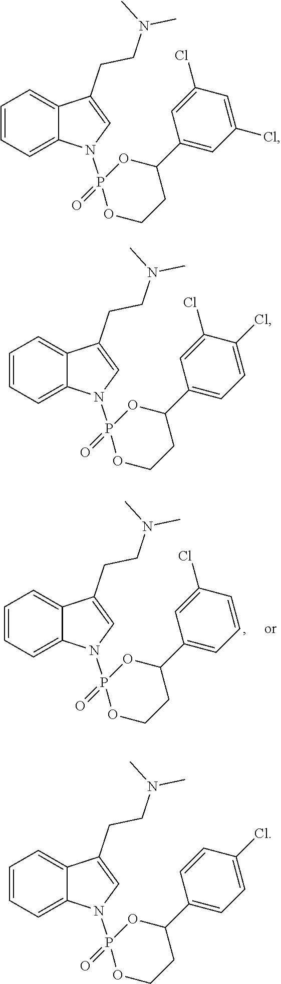

In some embodiments is a compound of Formula (I), (Io), or (Io1), having the structure of Formula (Io1a), or a pharmaceutically acceptable salt thereof:

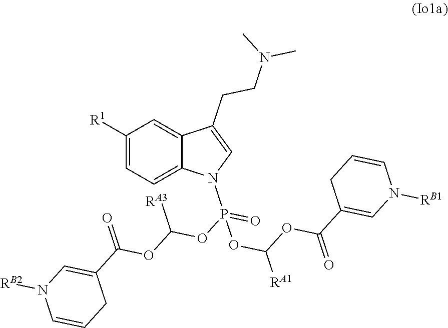

wherein:
R¹ is methoxy or hydrogen;
each of R^{A} and R^{A3} is independently hydrogen, alkyl, or cycloalkyl, wherein each alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R^{B1} and R^{B2} is independently hydrogen or alkyl that is unsubstituted or substituted with one or more halogen, amino, cyano, hydroxyl, alkyl, acetyl, or benzoyl.

In some embodiments is a compound of Formula (Io1a), or a pharmaceutically acceptable salt or solvate thereof, wherein each of R^{B1} and R^{B2} is independently hydrogen, methyl, ethyl, n-propyl, isopropyl, n-butyl, tert-butyl, pentan-3-yl, or benzyl. In some embodiments is a compound of Formula (Io1a), or a pharmaceutically acceptable salt or solvate thereof, wherein each of R^{A1} and R^{A3} is independently hydrogen, methyl, ethyl, isopropyl, or tert-butyl. In some embodiments is a compound of Formula (Io1a), or a pharmaceutically acceptable salt or solvate thereof, wherein each of R^{A1} and R^{A3} is independently hydrogen. In some embodiments is a compound of Formula (Io1a), or a pharmaceutically acceptable salt or solvate thereof, wherein each of R^{A1} and R^{A3} is independently hydrogen, methyl, ethyl, isopropyl, or tert-butyl; and each of R^{B1} and R^{B2} is hydrogen, methyl, ethyl, n-propyl, isopropyl, n-butyl, tert-butyl, pentan-3-yl, or benzyl.

In some embodiments is a compound of Formula (Io1a), or a pharmaceutically acceptable salt or solvate thereof, wherein the compound is:

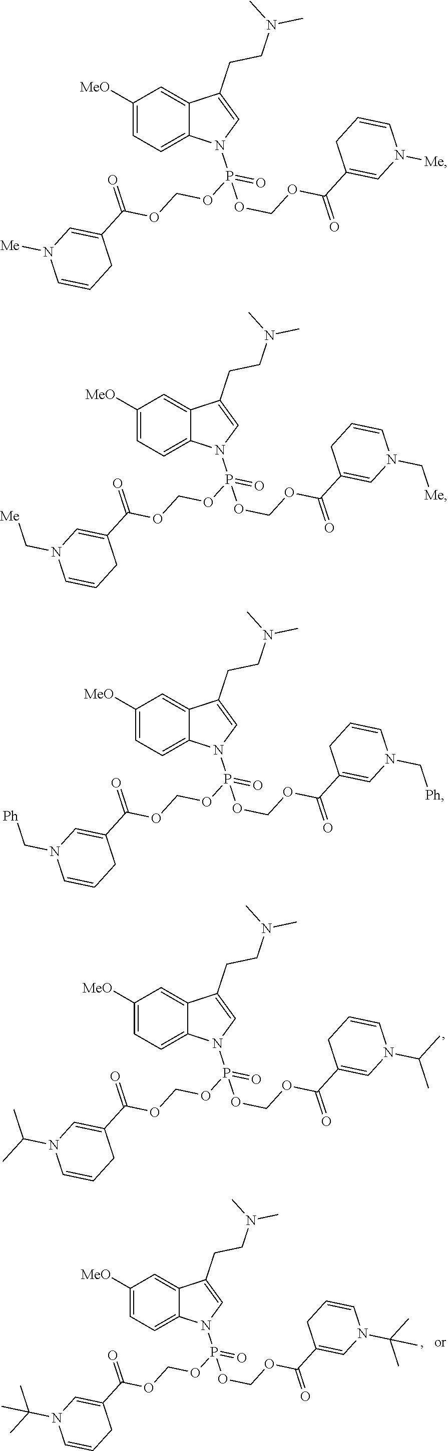

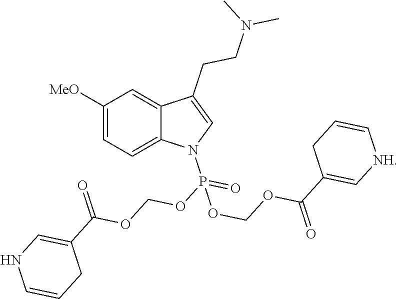 [SMILES: COc1ccc2c(c1)c(CCN(C)C)cn2P(=O)(OCOC(=O)C1=CNC=CC1)OCOC(=O)C1=CNC=CC1]

In some embodiments is a compound of Formula (Io1a), or a pharmaceutically acceptable salt or solvate thereof, wherein the compound is:

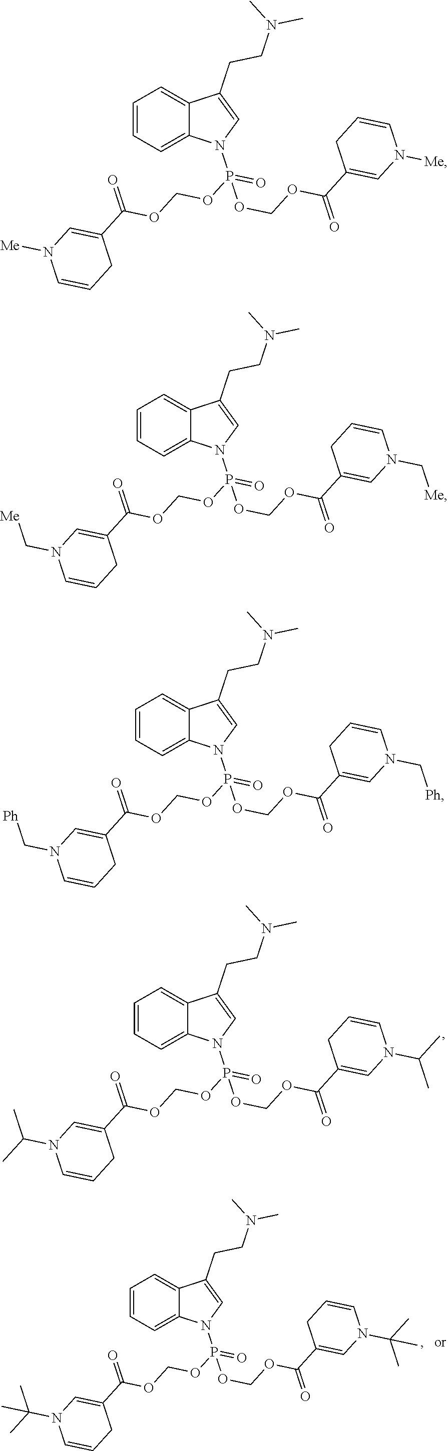

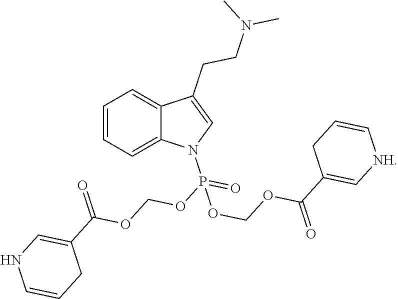 [SMILES: CN(C)CCc1cn(P(=O)(OCOC(=O)C2=CNC=CC2)OCOC(=O)C2=CNC=CC2)c2ccccc12]

In some embodiments is a compound of Formula (I) having the structure of Formula (Ip), or a pharmaceutically acceptable salt thereof:

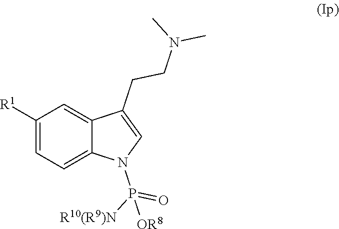

wherein:
R¹ is methoxy or hydrogen;
R⁸ is alkyl, cycloalkyl, aryl, heterocyclylalkyl, or heteroaryl; and
each of R⁹ and R¹⁰ is independently hydrogen or alkyl,
   wherein each alkyl, cycloalkyl, aryl, heterocyclylalkyl, and heteroaryl is independently unsubstituted or substituted with one or more R^{A}.

In some embodiments is a compound of Formula (I) or (Ip), or a pharmaceutically acceptable salt or solvate thereof, wherein R⁸ is alkyl or cycloalkyl. In some embodiments is a compound of Formula (I) or (Ip), or a pharmaceutically acceptable salt or solvate thereof, wherein R⁸ is unsubstituted alkyl or unsubstituted cycloalkyl. In some embodiments is a compound of Formula (I) or (Ip), or a pharmaceutically acceptable salt or solvate thereof, wherein R⁸ is methyl, ethyl, n-propyl, n-butyl, n-pentyl, n-hexyl, isopropyl, tert-butyl, cyclopropyl, cyclobutyl, cyclopentyl, cyclohexyl, or cycloheptyl. In some embodiments is a compound of Formula (I) or (Ip), or a pharmaceutically acceptable salt or solvate thereof, wherein R⁸ is aryl. In some embodiments is a compound of Formula (I) or (Ip), or a pharmaceutically acceptable salt or solvate thereof, wherein R⁸ is phenyl. In some embodiments is a compound of Formula (I) or (Ip), or a pharmaceutically acceptable salt or solvate thereof, wherein R⁸ is 4-nitrophenyl. In some embodiments is a compound of Formula (I) or (Ip), or a pharmaceutically acceptable salt or solvate thereof, wherein R⁸ is benzyl. In some embodiments is a compound of Formula (I) or (Ip), or a pharmaceutically acceptable salt or solvate thereof, wherein R⁸ is 2-pyridyl, 3-pyridyl, 4-pyridyl, 2-pyrimidyl, or 4-pyrimidyl. In some embodiments is a compound of Formula (I) or (Ip), or a pharmaceutically acceptable salt or solvate thereof, wherein R⁹ is hydrogen. In some embodiments is a compound of Formula (I) or (Ip), or a pharmaceutically acceptable salt or solvate thereof, wherein R⁹ is hydrogen, and R¹⁰ is alkyl.

In some embodiments is a compound of Formula (I) or (Ip) having the structure of Formula (Ip1), or a pharmaceutically acceptable salt thereof:

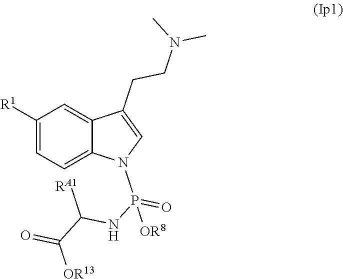

wherein:
R¹ is methoxy or hydrogen;
R^{A1} is hydrogen, alkyl, or cycloalkyl, wherein each of alkyl and cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹;
R⁸ is hydrogen, alkyl, cycloalkyl, aryl, heterocyclylalkyl, or heteroaryl, wherein each alkyl, cycloalkyl, aryl, heterocyclylalkyl, and heteroaryl is unsubstituted or substituted with one or more R^{A}; and
R¹³ is hydrogen or alkyl that is unsubstituted or substituted with one or more R^{B}.

In some embodiments is a compound of Formula (Ip1), or a pharmaceutically acceptable salt or solvate thereof, wherein R^{A1} is hydrogen or alkyl. In some embodiments is a compound of Formula (Ip1), or a pharmaceutically acceptable salt or solvate thereof, wherein R^{A1} is hydrogen or unsubstituted alkyl. In some embodiments is a compound of Formula (Ip1), or a pharmaceutically acceptable salt or solvate thereof, wherein R^{A1} is hydrogen, methyl, ethyl, or tert-butyl. In some embodiments is a compound of Formula (Ip1), or a pharmaceutically acceptable salt or solvate thereof, wherein R^{A1} is hydrogen. In some embodiments is a compound of Formula (Ip1), or a pharmaceutically acceptable salt or solvate thereof, wherein R⁸ is alkyl or cycloalkyl. In some embodiments is a compound of Formula (Ip1), or a pharmaceutically acceptable salt or solvate thereof, wherein R⁸ is unsubstituted alkyl or unsubstituted cycloalkyl. In some embodiments is a compound of Formula (Ip1), or a pharmaceutically acceptable salt or solvate thereof, wherein R⁸ is methyl, ethyl, n-propyl, n-butyl, n-pentyl, n-hexyl, isopropyl, tert-butyl, cyclopropyl, cyclobutyl, cyclopentyl, cyclohexyl, or cycloheptyl. In some embodiments is a compound of Formula (Ip1), or a pharmaceutically acceptable salt or solvate thereof, wherein R⁸ is phenyl. In some embodiments is a compound of Formula (Ip1), or a pharmaceutically acceptable salt or solvate thereof, wherein R⁸ is 4-nitrophenyl. In some embodiments is a compound of Formula (Ip1), or a pharmaceutically acceptable salt or solvate thereof, wherein R⁸ is benzyl. In some embodiments is a compound of Formula (Ip1), or a pharmaceutically acceptable salt or solvate thereof, wherein R⁸ is 2-pyridyl, 3-pyridyl, 4-pyridyl, 2-pyrimidyl, or 4-pyrimidyl. In some embodiments is a compound of Formula (Ip1), or a pharmaceutically acceptable salt or solvate thereof, wherein R¹³ is alkyl. In some embodiments is a compound of Formula (Ip1), or a pharmaceutically acceptable salt or solvate thereof, wherein R¹³ is unsubstituted alkyl. In some embodiments is a compound of Formula (Ip1), or a pharmaceutically acceptable salt or solvate thereof, wherein R¹³ is methyl, ethyl, n-propyl, isopropyl, n-butyl, tert-butyl, or —CH₂CH(Et)₂. In some embodiments is a compound of Formula (Ip1), or a pharmaceutically acceptable salt or solvate thereof, wherein R^{A1} is hydrogen or unsubstituted alkyl; and R¹³ is methyl, ethyl, n-propyl, isopropyl, n-butyl, tert-butyl, or —CH₂CH(Et)₂. In some embodiments is a compound of Formula (Ip1), or a pharmaceutically acceptable salt or solvate thereof, wherein R^{A1} is hydrogen; and R¹³ is methyl, ethyl, n-propyl, isopropyl, n-butyl, tert-butyl, or —CH₂CH(Et)₂.

In some embodiments is a compound of Formula (Ip1), or a pharmaceutically acceptable salt or solvate thereof, wherein the compound is:

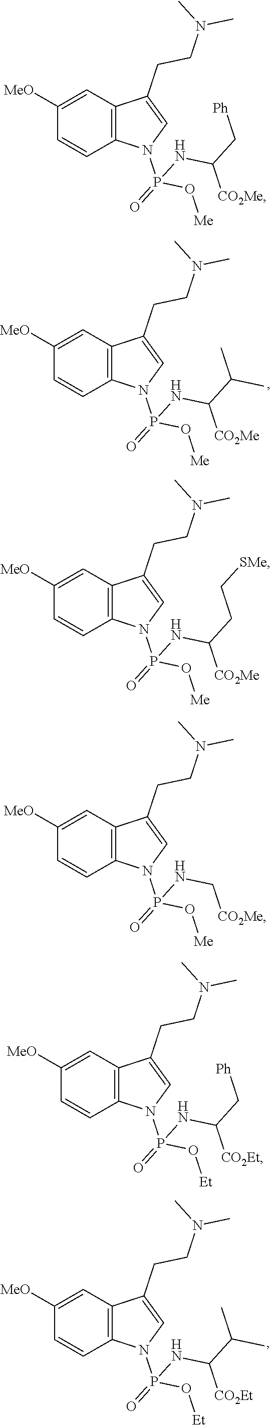

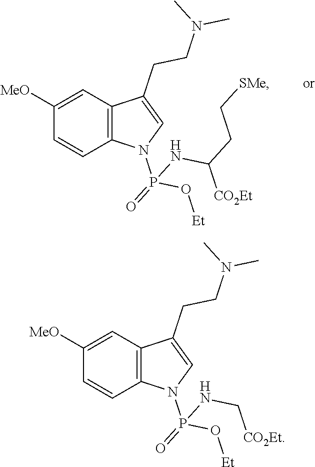

In some embodiments is a compound of Formula (Ip1), or a pharmaceutically

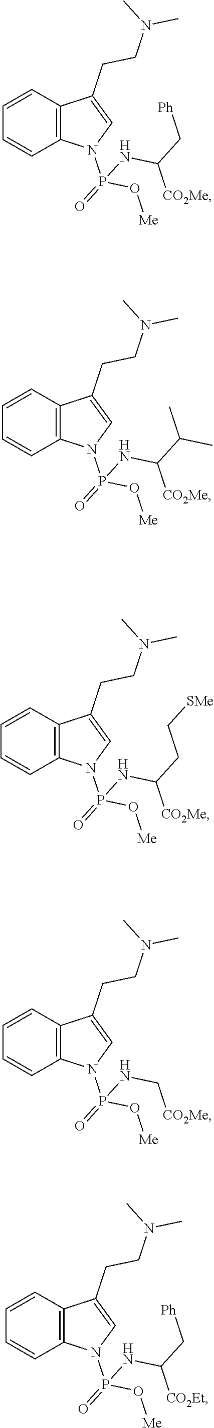 [SMILES: COC(=O)C(Cc1ccccc1)NP(=O)(OC)n1cc(CCN(C)C)c2ccccc21 ; COC(=O)C(NP(=O)(OC)n1cc(CCN(C)C)c2ccccc21)C(C)C ; COC(=O)C(CCSC)NP(=O)(OC)n1cc(CCN(C)C)c2ccccc21 ; COC(=O)CNP(=O)(OC)n1cc(CCN(C)C)c2ccccc21 ; CCOC(=O)C(Cc1ccccc1)NP(=O)(OC)n1cc(CCN(C)C)c2ccccc21]

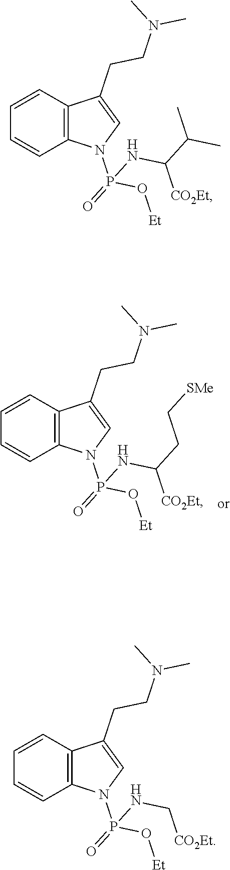 [SMILES: CCOC(=O)C(NP(=O)(OCC)n1cc(CCN(C)C)c2ccccc21)C(C)C ; CCOC(=O)C(CCSC)NP(=O)(OCC)n1cc(CCN(C)C)c2ccccc21 ; CCOC(=O)CNP(=O)(OCC)n1cc(CCN(C)C)c2ccccc21]

In some embodiments is a compound of Formula (I) having the structure of Formula (Iq), or a pharmaceutically acceptable salt thereof:

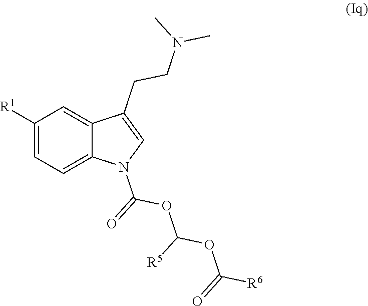

wherein:
R¹ is methoxy or hydrogen;
R⁵ is hydrogen, alkyl, or cycloalkyl; and
R⁶ is alkyl, cycloalkyl, heteroalkyl, heterocyclylalkyl, aryl, or heteroaryl,
   wherein each alkyl, cycloalkyl, heteroalkyl, heterocyclylalkyl, aryl, and heteroaryl is independently unsubstituted or substituted with one or more R^{A}.

In some embodiments is a compound of Formula (I) or (Iq), or a pharmaceutically acceptable salt or solvate thereof, wherein R⁵ is hydrogen or alkyl. In some embodiments is a compound of Formula (I) or (Iq), or a pharmaceutically acceptable salt or solvate thereof, wherein R⁵ is alkyl. In some embodiments is a compound of Formula (I) or (Iq), or a pharmaceutically acceptable salt or solvate thereof, wherein R⁵ is hydrogen or unsubstituted alkyl. In some embodiments is a compound of Formula (I) or (Iq), or a pharmaceutically acceptable salt or solvate thereof, wherein R⁵ is hydrogen. In some embodiments is a compound of Formula (I) or (Iq), or a pharmaceutically acceptable salt or solvate thereof, wherein R⁶ is alkyl, cycloalkyl, heterocyclylalkyl, aryl, or heteroaryl. In some embodiments is a compound of Formula (I) or (Iq), or a pharmaceutically acceptable salt or solvate thereof, wherein R⁶ is alkyl. In some embodiments is a compound of Formula (I) or (Iq), or a pharmaceutically acceptable salt or solvate thereof, wherein R⁶ is heterocyclylalkyl. In some embodiments is a compound of Formula (I) or (Iq), or a pharmaceutically acceptable salt or solvate thereof, wherein R⁶ is heteroalkyl. In some embodiments is a compound of Formula (I) or (Iq), or a pharmaceutically acceptable salt or solvate thereof, wherein R⁶ is unsubstituted alkyl, unsubstituted cycloalkyl, unsubstituted heterocyclylalkyl, unsubstituted aryl, or unsubstituted heteroaryl. In some embodiments is a compound of Formula (I) or (Iq), or a pharmaceutically acceptable salt or solvate thereof, wherein R⁶ is alkyl. In some embodiments is a compound of Formula (I) or (Iq), or a pharmaceutically acceptable salt or solvate thereof, wherein R⁶ is heteroalkyl. In some embodiments is a compound of Formula (I) or (Iq), or a pharmaceutically acceptable salt or solvate thereof, wherein R⁶ is heterocyclylalkyl substituted with arylalkyl. In some embodiments is a compound of Formula (I) or (Iq), or a pharmaceutically acceptable salt or solvate thereof, wherein R⁵ is methyl, isopropyl, tert-butyl, or —CH(Et)₂.

In some embodiments is a compound of Formula (I) or (Iq), or a pharmaceutically acceptable salt or solvate thereof, wherein R⁵ is hydrogen, and R⁶ is alkyl. In some embodiments is a compound of Formula (I) or (Iq), or a pharmaceutically acceptable salt or solvate thereof, wherein R⁵ is alkyl, and R⁶ is alkyl. In some embodiments is a compound of Formula (I) or (Iq), or a pharmaceutically acceptable salt or solvate thereof, wherein R⁵ is hydrogen, and R⁶ is unsubstituted alkyl. In some embodiments is a compound of Formula (I) or (Iq), or a pharmaceutically acceptable salt or solvate thereof, wherein R⁵ is unsubstituted alkyl, and R⁶ is unsubstituted alkyl. In some embodiments is a compound of Formula (I) or (Iq), or a pharmaceutically acceptable salt or solvate thereof, wherein R⁶ is methyl, ethyl, isopropyl, tert-butyl, or cyclopropyl. In some embodiments is a compound of Formula (I) or (Iq), or a pharmaceutically acceptable salt or solvate thereof, wherein R⁵ is hydrogen, and R⁶ is methyl, ethyl, isopropyl, tert-butyl, or cyclopropyl. In some embodiments is a compound of Formula (I) or (Iq), or a pharmaceutically acceptable salt or solvate thereof, wherein R⁵ is hydrogen, and R⁶ is tert-butyl. In some embodiments is a compound of Formula (I) or (Iq), or a pharmaceutically acceptable salt or solvate thereof, wherein R¹ is hydrogen, R⁵ is hydrogen, and R⁶ is tert-butyl. In some embodiments is a compound of Formula (I) or (Iq), or a pharmaceutically acceptable salt or solvate thereof, wherein R¹¹ is methoxy, R⁵ is hydrogen, and R⁶ is tert-butyl.

In some embodiments is a compound of Formula (I) or (Iq), or a pharmaceutically acceptable salt or solvate thereof, wherein R⁶ is alkyl. In some embodiments is a compound of Formula (I) or (Iq), or a pharmaceutically acceptable salt or solvate thereof, wherein R⁶ is unsubstituted alkyl. In some embodiments is a compound of Formula (I) or (Iq), or a pharmaceutically acceptable salt or solvate thereof, wherein R⁶ is cycloalkyl. In some embodiments is a compound of Formula (I) or (Iq), or a pharmaceutically acceptable salt or solvate thereof, wherein R⁶ is methyl, ethyl, n-propyl, tert-butyl, 3-methyl-1-butyl, n-pentyl, n-hexyl, cyclopropyl, cyclobutyl, cyclopentyl, cyclohexyl, or cycloheptyl. In some embodiments is a compound of Formula (I) or (Iq), or a pharmaceutically acceptable salt or solvate thereof, wherein R⁶ is phenyl. In some embodiments is a compound of Formula (I) or (Iq), or a pharmaceutically acceptable salt or solvate thereof, wherein R⁶ is 4-nitrophenyl. In some embodiments is a compound of Formula (I) or (Iq), or a pharmaceutically acceptable salt or solvate thereof, wherein R⁶ is benzyl. In some embodiments is a compound of Formula (I) or (Iq), or a pharmaceutically acceptable salt or solvate thereof, wherein R⁶ is heteroaryl. In some embodiments is a compound of Formula (I) or (Iq), or a pharmaceutically acceptable salt or solvate thereof, wherein R⁶ is 2-pyridyl, 3-pyridyl, 4-pyridyl, 2-pyrimidyl, or 4-pyrimidyl.

In some embodiments is a compound of Formula (I) or (Iq), or a pharmaceutically acceptable salt or solvate thereof, wherein R⁶ is heteroalkyl. In some embodiments is a compound of Formula (I) or (Iq), or a pharmaceutically acceptable salt or solvate thereof, wherein R⁶ is CH₂CH₂OMe or CH₂CH₂SO₂Me. In some embodiments is a compound of Formula (I) or (Iq), or a pharmaceutically acceptable salt or solvate thereof, wherein R⁶ is —(CH₂)ᵣCO₂H, wherein r is 1, 2, 3, 4, 5, or 6. In some embodiments is a compound of Formula (I) or (Iq), or a pharmaceutically acceptable salt or solvate thereof, wherein R⁶ is —(CH₂)ₛCO₂R¹³, wherein s is 1, 2, 3, 4, 5, or 6. In some embodiments is a compound of Formula (I) or (Iq), or a pharmaceutically acceptable salt or solvate thereof, wherein R⁶ is —(CH₂)ₛCO₂R¹³, wherein R¹³ is alkyl. In some embodiments is a compound of Formula (I) or (Iq), or a pharmaceutically acceptable salt or solvate thereof, wherein R⁶ is —(CH₂)ₛCO₂R¹³, wherein R¹³ is unsubstituted alkyl. In some embodiments is a compound of Formula (I) or (Iq), or a pharmaceutically acceptable salt or solvate thereof, wherein R⁶ is —(CH₂)ₛCO₂R¹³, wherein R¹³ is methyl, ethyl, n-propyl, isopropyl, n-butyl, tert-butyl, or —CH(Et)₂.

In some embodiments is a compound of Formula (I) or (Iq), or a pharmaceutically acceptable salt or solvate thereof, wherein R⁶ is —CH(R^{A1})NH₂, wherein R^{A1} is hydrogen, alkyl, heteroalkyl, or an amino acid side chain. In some embodiments is a compound of Formula (I) or (Iq), or a pharmaceutically acceptable salt or solvate thereof, wherein R⁶ is —CH(R^{A1})NH₂, wherein R^{A1} is an amino acid side chain. In some embodiments is a compound of Formula (I) or (Iq), or a pharmaceutically acceptable salt or solvate thereof, wherein R⁶ is —CH(R^{A1})NH₂, wherein R^{A1} is methyl, ethyl, n-propyl, isopropyl, tert-butyl, CH(Me)Et, CH₂CH(Me)₂, or CH₂CH₂SMe. In some embodiments is a compound of Formula (I) or (Iq), or a pharmaceutically acceptable salt or solvate thereof, wherein R⁶ is —CH(R^{A1})NH₂, wherein R^{A1} is benzyl.

In some embodiments is a compound of Formula (I) or (Iq), or a pharmaceutically acceptable salt or solvate thereof, wherein the compound is:

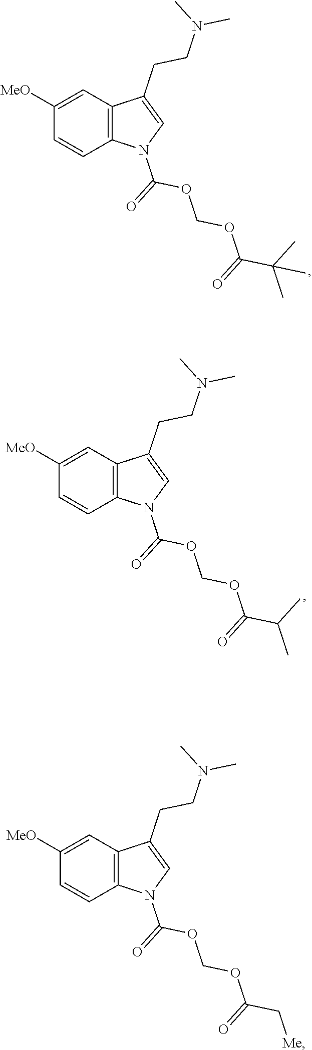 [SMILES: COc1ccc2c(c1)c(CCN(C)C)cn2C(=O)OCOC(=O)C(C)(C)C ; COc1ccc2c(c1)c(CCN(C)C)cn2C(=O)OCOC(=O)C(C)C ; CCC(=O)OCOC(=O)n1cc(CCN(C)C)c2cc(OC)ccc21]

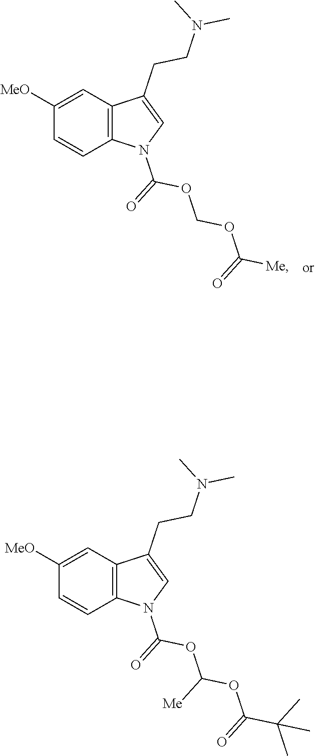 [SMILES: COc1ccc2c(c1)c(CCN(C)C)cn2C(=O)OCOC(C)=O ; COc1ccc2c(c1)c(CCN(C)C)cn2C(=O)OC(C)OC(=O)C(C)(C)C]

In some embodiments is a compound of Formula (I) or (Iq), or a pharmaceutically acceptable salt or solvate thereof, wherein the compound is:

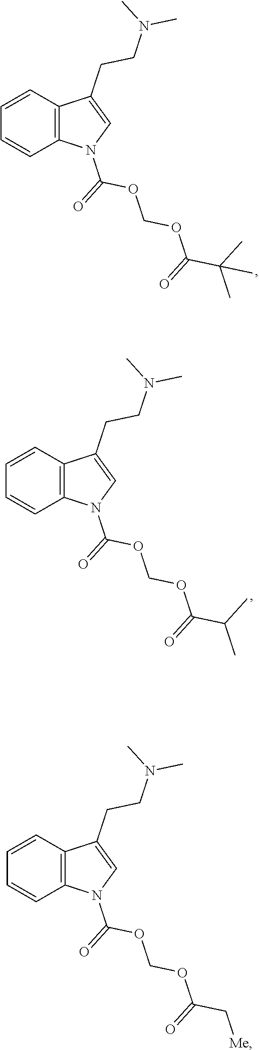 [SMILES: CN(C)CCc1cn(C(=O)OCOC(=O)C(C)(C)C)c2ccccc12 ; CC(C)C(=O)OCOC(=O)n1cc(CCN(C)C)c2ccccc21 ; CCC(=O)OCOC(=O)n1cc(CCN(C)C)c2ccccc21]

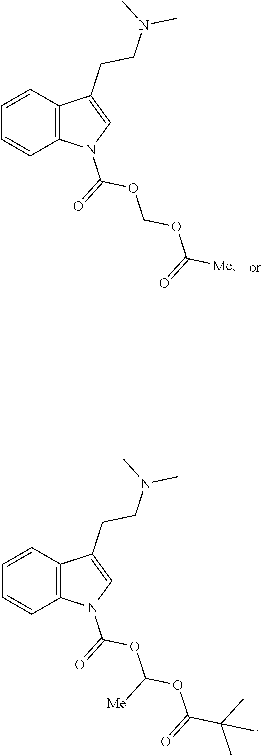 [SMILES: CC(=O)OCOC(=O)n1cc(CCN(C)C)c2ccccc21 ; CC(OC(=O)n1cc(CCN(C)C)c2ccccc21)OC(=O)C(C)(C)C]

In some embodiments is a compound of Formula (I) or (Iq), or a pharmaceutically acceptable salt or solvate thereof, wherein the compound is:

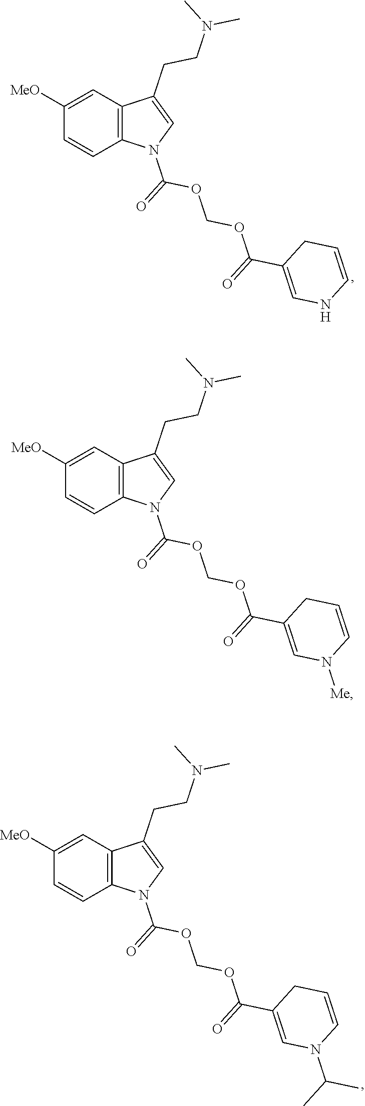 [SMILES: COc1ccc2c(c1)c(CCN(C)C)cn2C(=O)OCOC(=O)C1=CNC=CC1 ; COc1ccc2c(c1)c(CCN(C)C)cn2C(=O)OCOC(=O)C1=CN(C)C=CC1 ; COc1ccc2c(c1)c(CCN(C)C)cn2C(=O)OCOC(=O)C1=CN(C(C)C)C=CC1]

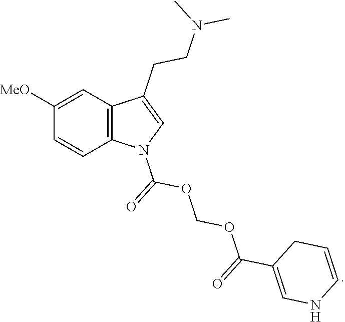 [SMILES: COc1ccc2c(c1)c(CCN(C)C)cn2C(=O)OCOC(=O)C1=CNC=CC1]

In some embodiments is a compound of Formula (I) or (Iq), or a pharmaceutically

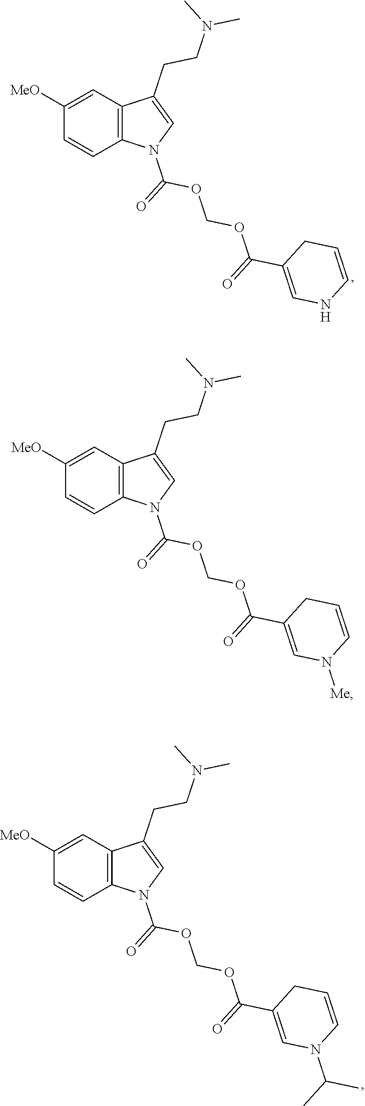 [SMILES: COc1ccc2c(c1)c(CCN(C)C)cn2C(=O)OCOC(=O)C1=CNC=CC1 ; COc1ccc2c(c1)c(CCN(C)C)cn2C(=O)OCOC(=O)C1=CN(C)C=CC1 ; COc1ccc2c(c1)c(CCN(C)C)cn2C(=O)OCOC(=O)C1=CN(C(C)C)C=CC1]

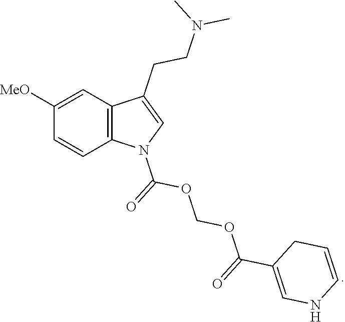 [SMILES: COc1ccc2c(c1)c(CCN(C)C)cn2C(=O)OCOC(=O)C1=CNC=CC1]

In some embodiments is a compound of Formula (I) or (Iq), or a pharmaceutically acceptable salt or solvate thereof, wherein the compound is:

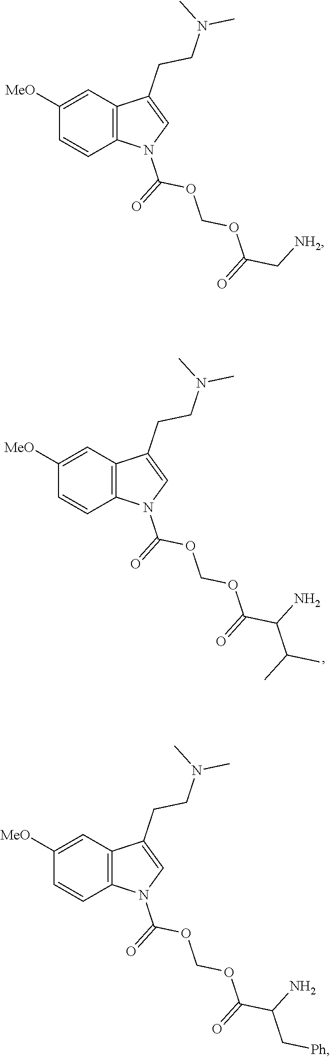 [SMILES: COc1ccc2c(c1)c(CCN(C)C)cn2C(=O)OCOC(=O)CN ; COc1ccc2c(c1)c(CCN(C)C)cn2C(=O)OCOC(=O)C(N)C(C)C ; COc1ccc2c(c1)c(CCN(C)C)cn2C(=O)OCOC(=O)C(N)Cc1ccccc1]

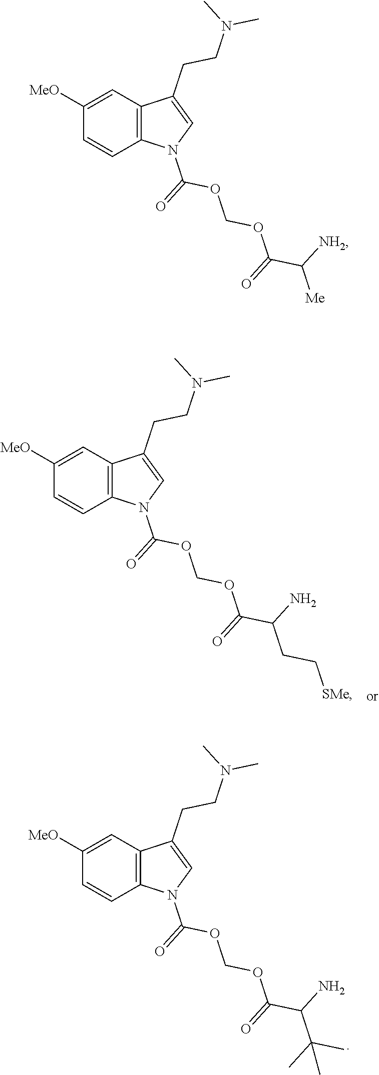 [SMILES: COc1ccc2c(c1)c(CCN(C)C)cn2C(=O)OCOC(=O)C(C)N ; COc1ccc2c(c1)c(CCN(C)C)cn2C(=O)OCOC(=O)C(N)CCSC ; COc1ccc2c(c1)c(CCN(C)C)cn2C(=O)OCOC(=O)C(N)C(C)(C)C]

In some embodiments is a compound of Formula (I) or (Iq), or a pharmaceutically acceptable salt or solvate thereof, wherein the compound is:

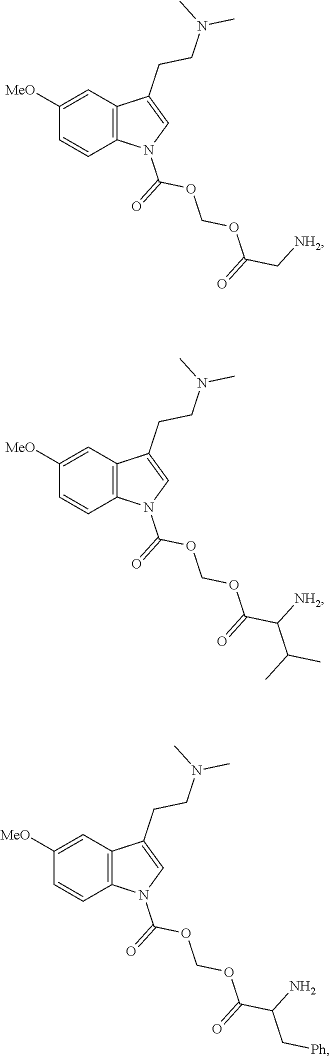 [SMILES: COc1ccc2c(c1)c(CCN(C)C)cn2C(=O)OCOC(=O)CN ; COc1ccc2c(c1)c(CCN(C)C)cn2C(=O)OCOC(=O)C(N)C(C)C ; COc1ccc2c(c1)c(CCN(C)C)cn2C(=O)OCOC(=O)C(N)Cc1ccccc1]

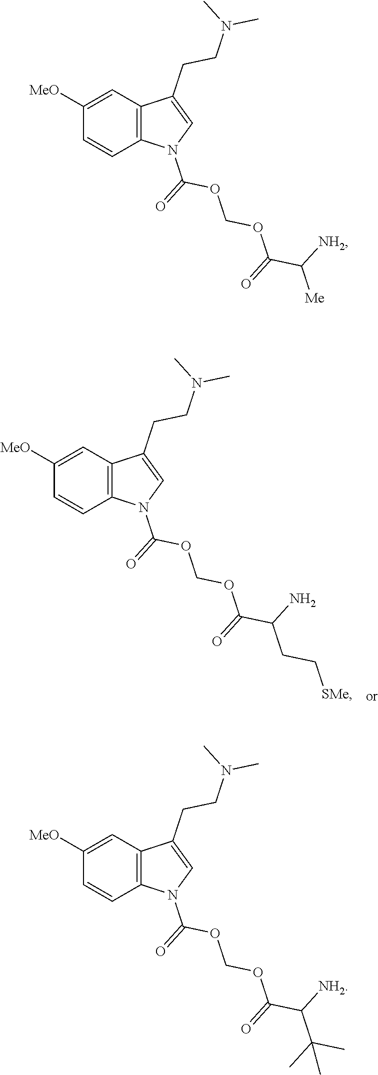 [SMILES: COc1ccc2c(c1)c(CCN(C)C)cn2C(=O)OCOC(=O)C(C)N ; COc1ccc2c(c1)c(CCN(C)C)cn2C(=O)OCOC(=O)C(N)CCSC ; COc1ccc2c(c1)c(CCN(C)C)cn2C(=O)OCOC(=O)C(N)C(C)(C)C]

In some embodiments is a compound of Formula (I) or (Iq) having the structure of Formula (Iq1), or a pharmaceutically acceptable salt thereof:

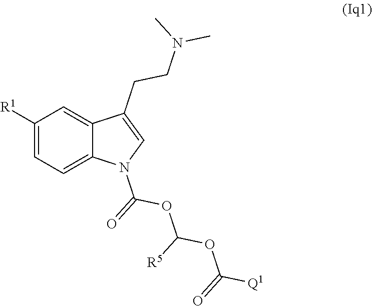

wherein:
R¹ is methoxy or hydrogen;
R⁵ is hydrogen, alkyl, or cycloalkyl, wherein each of alkyl and cycloalkyl is unsubstituted or substituted with one or more R^{A}; and
Q is

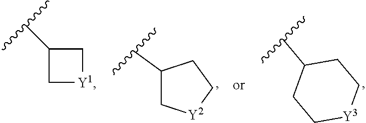

wherein
each of Y¹, Y², or Y³ is independently —O—, —S—, —S(O)—, —S(O)₂—, —N(R^{Y1})—, or —NC(O)R^{Y2}, wherein each of R^{Y1} and R^{Y2} is independently hydrogen, alkyl, heteroalkyl, or heteroaryl.

In some embodiments is a compound of Formula (Iq1), or a pharmaceutically acceptable salt or solvate thereof, wherein each of Y¹, Y², or Y³ is —N(R^{Y1})—. In some embodiments is a compound of Formula (Iq1), or a pharmaceutically acceptable salt or solvate thereof, wherein each of Y¹, Y², or Y³ is —N(R^{Y1})—, wherein R^{Y1} is hydrogen. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently methyl, ethyl, n-propyl, isopropyl, n-butyl, tert-butyl, CH(Et)₂, CH₂CH₂OMe, CH₂CH₂SO₂Me, or CH₂CF₃. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is phenyl. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is benzyl. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently 2-pyridyl, 3-pyridyl, or 4-pyridyl.

In some embodiments is a compound of Formula (Iq) or (Iq1), or a pharmaceutically acceptable salt or solvate thereof, wherein the compound is:

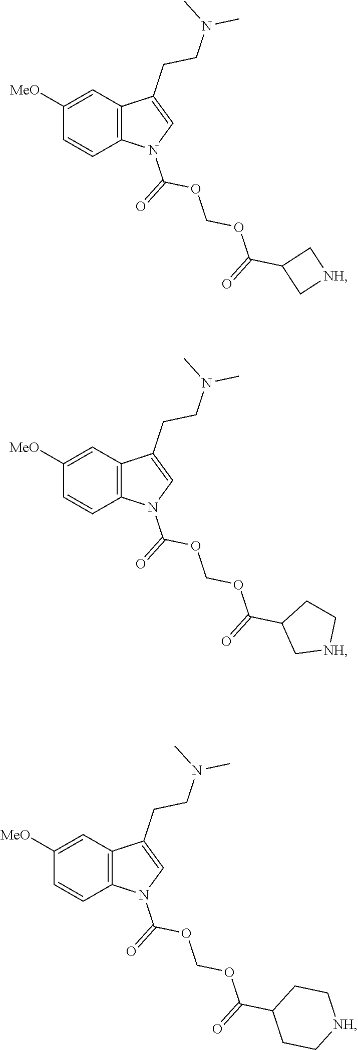 [SMILES: COc1ccc2c(c1)c(CCN(C)C)cn2C(=O)OCOC(=O)C1CNC1 ; COc1ccc2c(c1)c(CCN(C)C)cn2C(=O)OCOC(=O)C1CCNC1 ; COc1ccc2c(c1)c(CCN(C)C)cn2C(=O)OCOC(=O)C1CCNCC1]

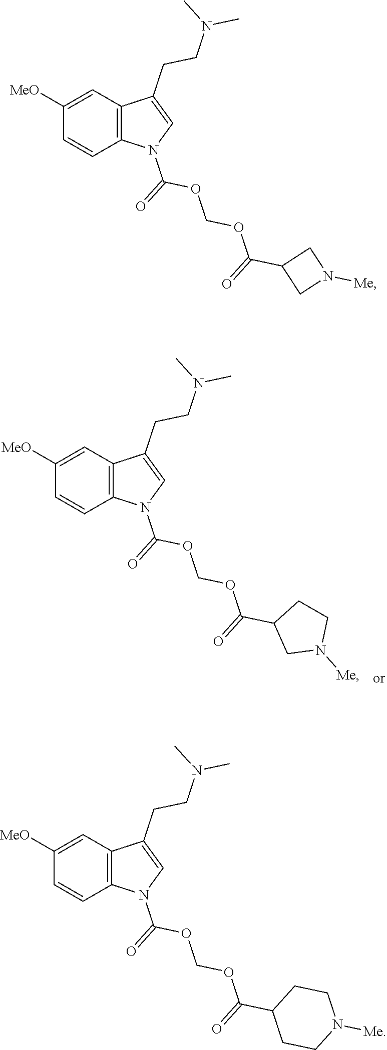 [SMILES: COc1ccc2c(c1)c(CCN(C)C)cn2C(=O)OCOC(=O)C1CN(C)C1 ; COc1ccc2c(c1)c(CCN(C)C)cn2C(=O)OCOC(=O)C1CCN(C)C1 ; COc1ccc2c(c1)c(CCN(C)C)cn2C(=O)OCOC(=O)C1CCN(C)CC1]

In some embodiments is a compound of Formula (Iq) or (Iq1), or a pharmaceutically acceptable salt or solvate thereof, wherein the compound is:

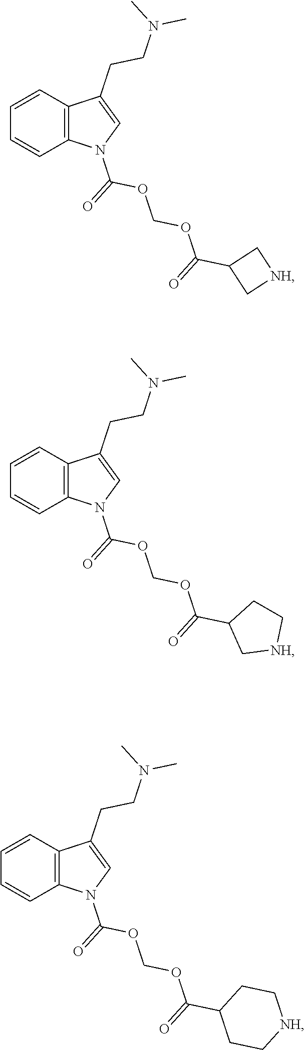 [SMILES: CN(C)CCc1cn(C(=O)OCOC(=O)C2CNC2)c2ccccc12 ; CN(C)CCc1cn(C(=O)OCOC(=O)C2CCNC2)c2ccccc12 ; CN(C)CCc1cn(C(=O)OCOC(=O)C2CCNCC2)c2ccccc12]

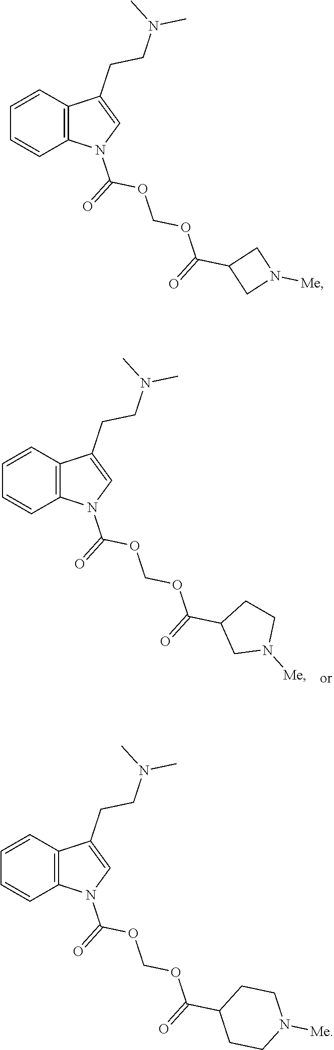 [SMILES: CN(C)CCc1cn(C(=O)OCOC(=O)C2CN(C)C2)c2ccccc12 ; CN(C)CCc1cn(C(=O)OCOC(=O)C2CCN(C)C2)c2ccccc12 ; CN(C)CCc1cn(C(=O)OCOC(=O)C2CCN(C)CC2)c2ccccc12]

In some embodiments is a compound of Formula (Iq) or (Iq1), or a pharmaceutically acceptable salt or solvate thereof, wherein the compound is:

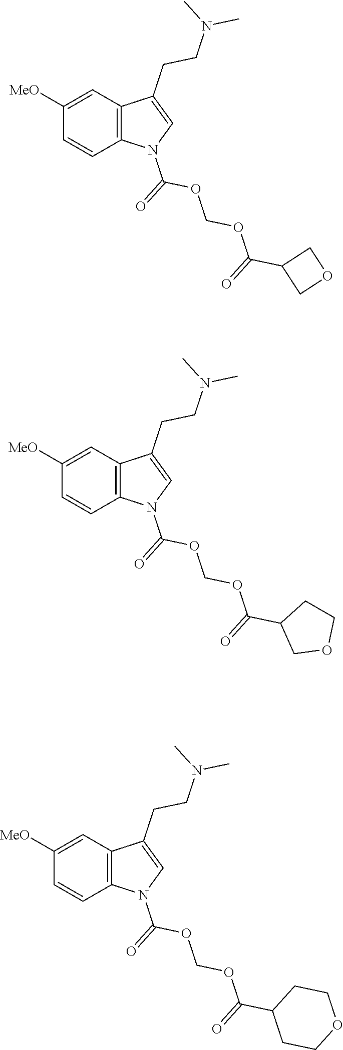 [SMILES: COc1ccc2c(c1)c(CCN(C)C)cn2C(=O)OCOC(=O)C1COC1 ; COc1ccc2c(c1)c(CCN(C)C)cn2C(=O)OCOC(=O)C1CCOC1 ; COc1ccc2c(c1)c(CCN(C)C)cn2C(=O)OCOC(=O)C1CCOCC1]

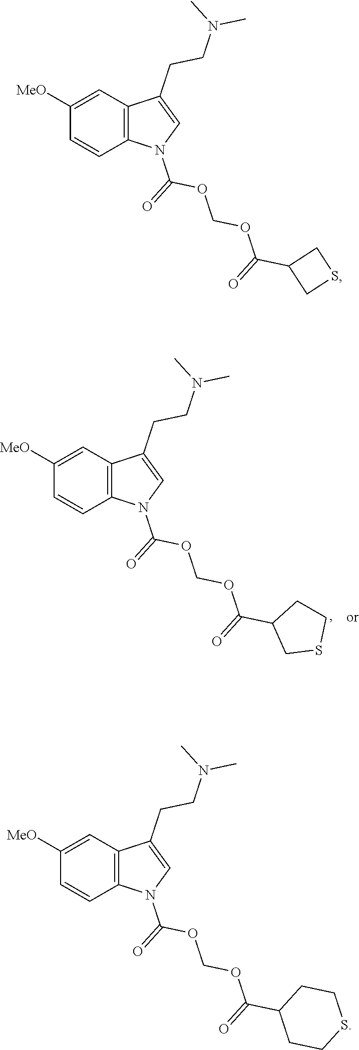 [SMILES: COc1ccc2c(c1)c(CCN(C)C)cn2C(=O)OCOC(=O)C1CSC1 ; COc1ccc2c(c1)c(CCN(C)C)cn2C(=O)OCOC(=O)C1CCSC1 ; COc1ccc2c(c1)c(CCN(C)C)cn2C(=O)OCOC(=O)C1CCSCC1]

In some embodiments is a compound of Formula (Iq) or (Iq1), or a pharmaceutically acceptable salt or solvate thereof, wherein the compound is:

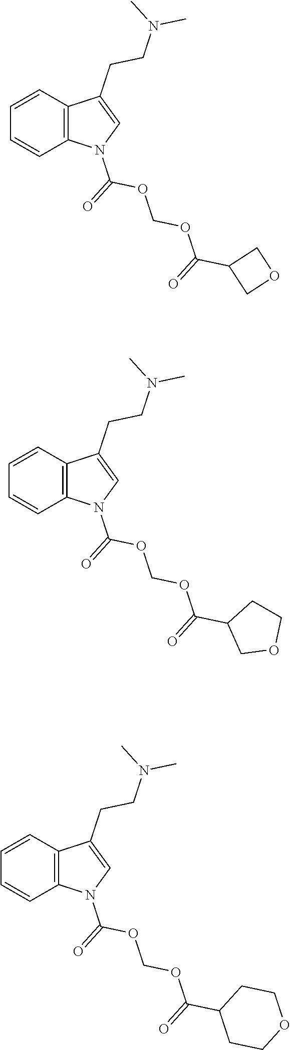 [SMILES: CN(C)CCc1cn(C(=O)OCOC(=O)C2COC2)c2ccccc12 ; CN(C)CCc1cn(C(=O)OCOC(=O)C2CCOC2)c2ccccc12 ; CN(C)CCc1cn(C(=O)OCOC(=O)C2CCOCC2)c2ccccc12]

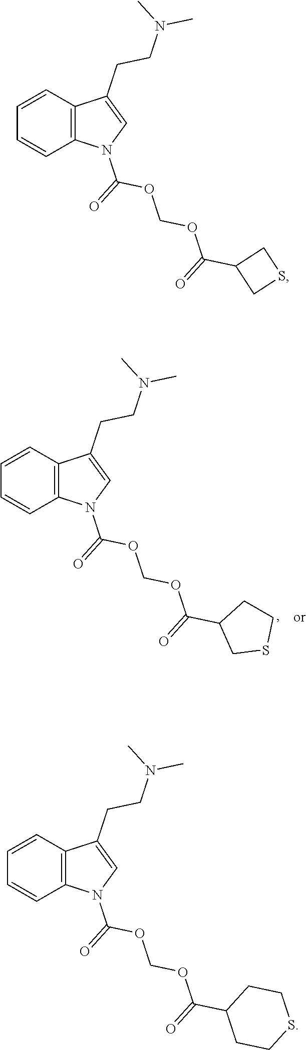 [SMILES: CN(C)CCc1cn(C(=O)OCOC(=O)C2CSC2)c2ccccc12 ; CN(C)CCc1cn(C(=O)OCOC(=O)C2CCSC2)c2ccccc12 ; CN(C)CCc1cn(C(=O)OCOC(=O)C2CCSCC2)c2ccccc12]

In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

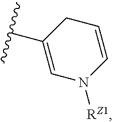

wherein R^{Z1} is hydrogen or alkyl. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

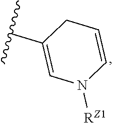

wherein R^{Z1} is methyl, ethyl, n-propyl, isopropyl, n-butyl, tert-butyl, or CH(Et)₂. In some embodiments is a compound of Formula (Iq1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

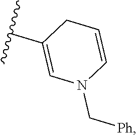

wherein R^{Z1} is benzyl.

In some embodiments is a compound of Formula (Iq1), or a pharmaceutically acceptable salt or solvate thereof, wherein the compound is:

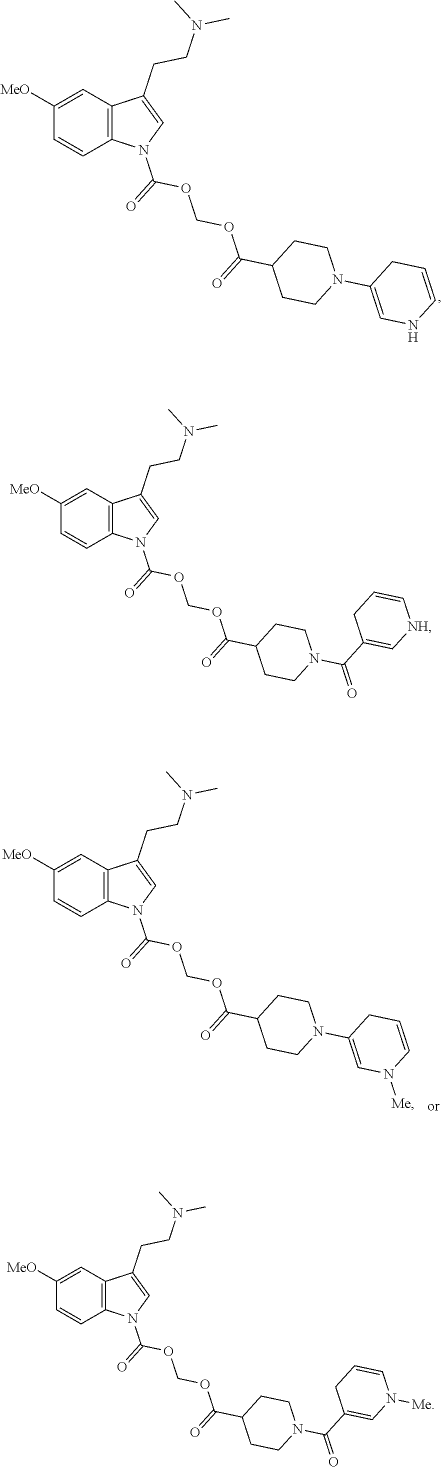 [SMILES: COc1ccc2c(c1)c(CCN(C)C)cn2C(=O)OCOC(=O)C1CCN(C2=CNC=CC2)CC1 ; COc1ccc2c(c1)c(CCN(C)C)cn2C(=O)OCOC(=O)C1CCN(C(=O)C2=CNC=CC2)CC1 ; COc1ccc2c(c1)c(CCN(C)C)cn2C(=O)OCOC(=O)C1CCN(C2=CN(C)C=CC2)CC1 ; COc1ccc2c(c1)c(CCN(C)C)cn2C(=O)OCOC(=O)C1CCN(C(=O)C2=CN(C)C=CC2)CC1]

In some embodiments is a compound of Formula (Iq1), or a pharmaceutically acceptable salt or solvate thereof, wherein the compound is:

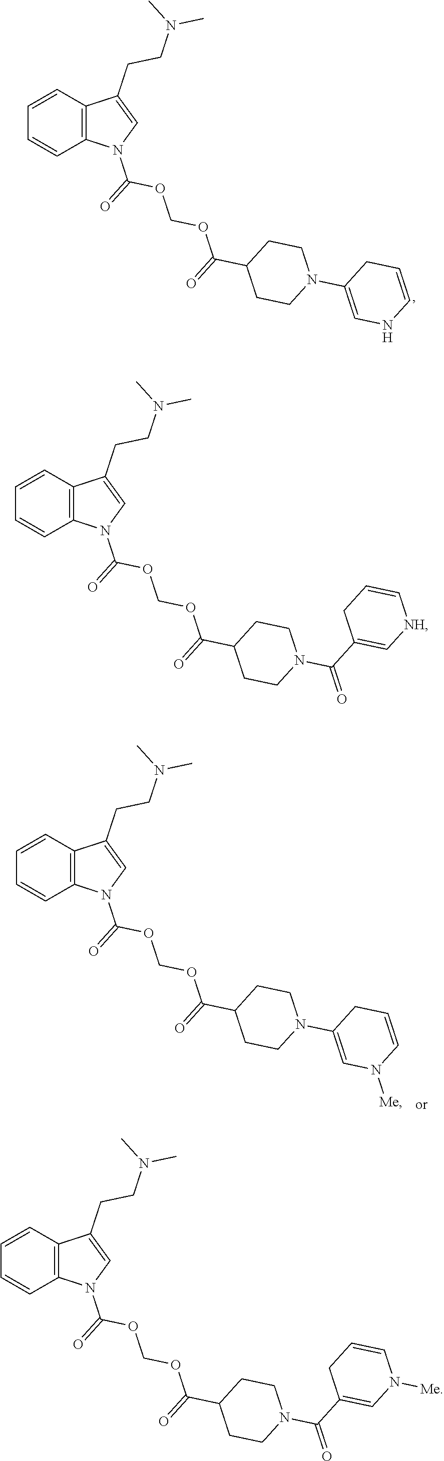 [SMILES: CN(C)CCc1cn(C(=O)OCOC(=O)C2CCN(C3=CNC=CC3)CC2)c2ccccc12 ; CN(C)CCc1cn(C(=O)OCOC(=O)C2CCN(C(=O)C3=CNC=CC3)CC2)c2ccccc12 ; CN1C=CCC(N2CCC(C(=O)OCOC(=O)n3cc(CCN(C)C)c4ccccc43)CC2)=C1 ; CN1C=CCC(C(=O)N2CCC(C(=O)OCOC(=O)n3cc(CCN(C)C)c4ccccc43)CC2)=C1]

In some embodiments is a compound of Formula (I) having the structure of Formula (Ir), or a pharmaceutically acceptable salt thereof:

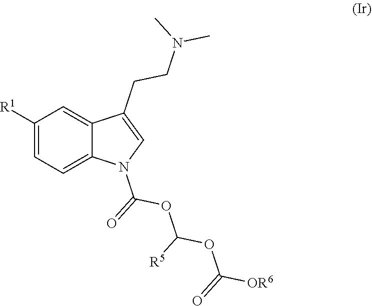

wherein:
R¹ is methoxy or hydrogen;
R⁵ is hydrogen, alkyl, or cycloalkyl; and
R⁶ is alkyl, cycloalkyl, heteroalkyl, heterocyclylalkyl, aryl, or heteroaryl,
   wherein each alkyl, cycloalkyl, heteroalkyl, heterocyclylalkyl, aryl, and heteroaryl is independently unsubstituted or substituted with one or more R^{A}.

In some embodiments is a compound of Formula (I) or (Ir), or a pharmaceutically acceptable salt or solvate thereof, wherein R⁵ is hydrogen or alkyl. In some embodiments is a compound of Formula (I) or (Ir), or a pharmaceutically acceptable salt or solvate thereof, wherein R⁵ is alkyl. In some embodiments is a compound of Formula (I) or (Ir), or a pharmaceutically acceptable salt or solvate thereof, wherein R⁵ is hydrogen. In some embodiments is a compound of Formula (I) or (Ir), or a pharmaceutically acceptable salt or solvate thereof, wherein R⁵ is hydrogen or unsubstituted alkyl. In some embodiments is a compound of Formula (I) or (Ir), or a pharmaceutically acceptable salt or solvate thereof, wherein R⁵ is hydrogen. In some embodiments is a compound of Formula (I) or (Ir), or a pharmaceutically acceptable salt or solvate thereof, wherein R⁶ is alkyl, cycloalkyl, heterocyclylalkyl, aryl, or heteroaryl. In some embodiments is a compound of Formula (I) or (Ir), or a pharmaceutically acceptable salt or solvate thereof, wherein R⁶ is alkyl. In some embodiments is a compound of Formula (I) or (Ir), or a pharmaceutically acceptable salt or solvate thereof, wherein R⁶ is heteroalkyl. In some embodiments is a compound of Formula (I) or (Ir), or a pharmaceutically acceptable salt or solvate thereof, wherein R⁶ is unsubstituted alkyl, unsubstituted cycloalkyl, unsubstituted heterocyclylalkyl, unsubstituted aryl, or unsubstituted heteroaryl. In some embodiments is a compound of Formula (I) or (Ir), or a pharmaceutically acceptable salt or solvate thereof, wherein R⁶ is alkyl. In some embodiments is a compound of Formula (I) or (Ir), or a pharmaceutically acceptable salt or solvate thereof, wherein R⁶ is heteroalkyl. In some embodiments is a compound of Formula (I) or (Ir), or a pharmaceutically acceptable salt or solvate thereof, wherein R⁶ is heterocyclylalkyl substituted with arylalkyl. In some embodiments is a compound of Formula (I) or (Ir), or a pharmaceutically acceptable salt or solvate thereof, wherein R⁵ is methyl, ethyl, isopropyl, tert-butyl, or —CH(Et)₂.

In some embodiments is a compound of Formula (I) or (Ir), or a pharmaceutically acceptable salt or solvate thereof, wherein R⁵ is hydrogen, and R⁶ is alkyl. In some embodiments is a compound of Formula (I) or (Ir), or a pharmaceutically acceptable salt or solvate thereof, wherein R⁵ is alkyl, and R⁶ is alkyl. In some embodiments is a compound of Formula (I) or (Ir), or a pharmaceutically acceptable salt or solvate thereof, wherein R⁵ is hydrogen, and R⁶ is unsubstituted alkyl. In some embodiments is a compound of Formula (I) or (Ir), or a pharmaceutically acceptable salt or solvate thereof, wherein R⁵ is unsubstituted alkyl, and R⁶ is unsubstituted alkyl. In some embodiments is a compound of Formula (I) or (Ir), or a pharmaceutically acceptable salt or solvate thereof, wherein R⁵ is unsubstituted alkyl, and R⁶ is heterocyclylalkyl. In some embodiments is a compound of Formula (I) or (Ir), or a pharmaceutically acceptable salt or solvate thereof, wherein R⁶ is methyl, ethyl, n-propyl, isopropyl, tert-butyl, 3-methyl-1-butyl, n-pentyl, n-hexyl, cyclopropyl, cyclobutyl, cyclopentyl, cyclohexyl, or cycloheptyl. In some embodiments is a compound of Formula (I) or (Ir), or a pharmaceutically acceptable salt or solvate thereof, wherein R⁶ is aryl. In some embodiments is a compound of Formula (I) or (Ir), or a pharmaceutically acceptable salt or solvate thereof, wherein R⁶ is phenyl. In some embodiments is a compound of Formula (I) or (Ir), or a pharmaceutically acceptable salt or solvate thereof, wherein R⁶ is heterocyclylalkyl. In some embodiments is a compound of Formula (I) or (Ir), or a pharmaceutically acceptable salt or solvate thereof, wherein R⁶ is oxetan-3-yl or azetindin-3-yl. In some embodiments is a compound of Formula (I) or (Ir), or a pharmaceutically acceptable salt or solvate thereof, wherein R⁶ is heteroaryl. In some embodiments is a compound of Formula (I) or (Ir), or a pharmaceutically acceptable salt or solvate thereof, wherein R⁶ is 2-pyridyl, 3-pyridyl, 4-pyridyl, 2-pyrimidyl, or 4-pyrimidyl. In some embodiments is a compound of Formula (I) or (Ir), or a pharmaceutically acceptable salt or solvate thereof, wherein R⁶ is benzyl. In some embodiments is a compound of Formula (I) or (Ir), or a pharmaceutically acceptable salt or solvate thereof, wherein R⁶ is

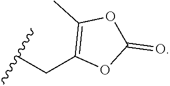 [SMILES: *Cc1oc(=O)oc1C]

In some embodiments is a compound of Formula (I) or (Ir), or a pharmaceutically acceptable salt or solvate thereof, wherein the compound is:

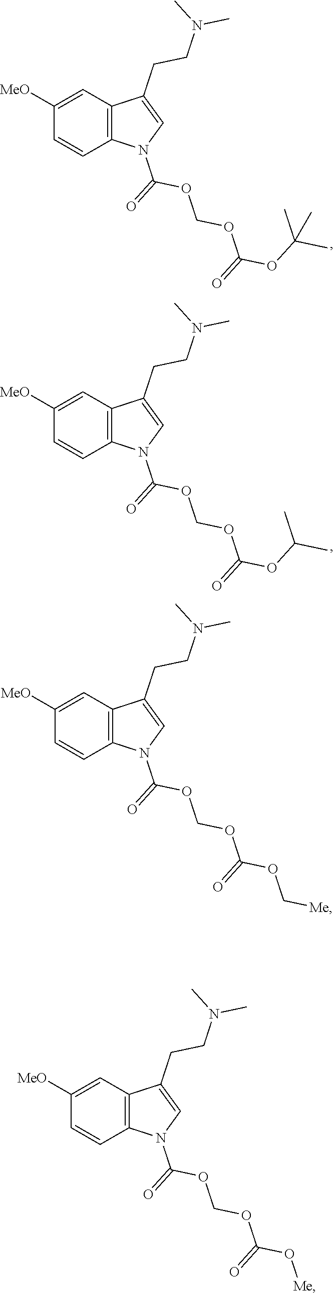

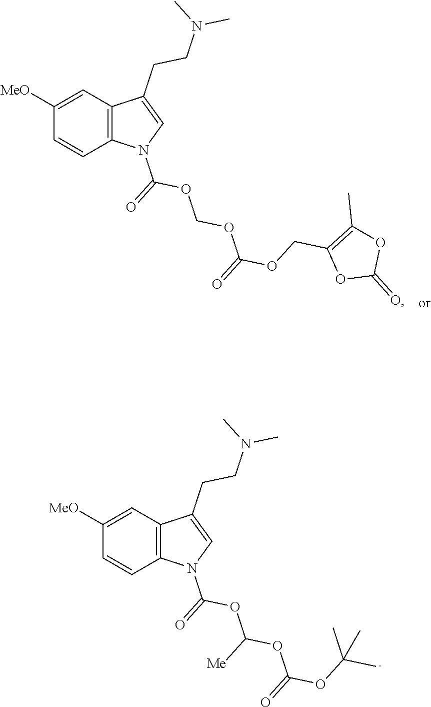 [SMILES: COc1ccc2c(c1)c(CCN(C)C)cn2C(=O)OCOC(=O)OCc1oc(=O)oc1C ; COc1ccc2c(c1)c(CCN(C)C)cn2C(=O)OC(C)OC(=O)OC(C)(C)C]

In some embodiments is a compound of Formula (I) or (Ir), or a pharmaceutically acceptable salt or solvate thereof, wherein the compound is:

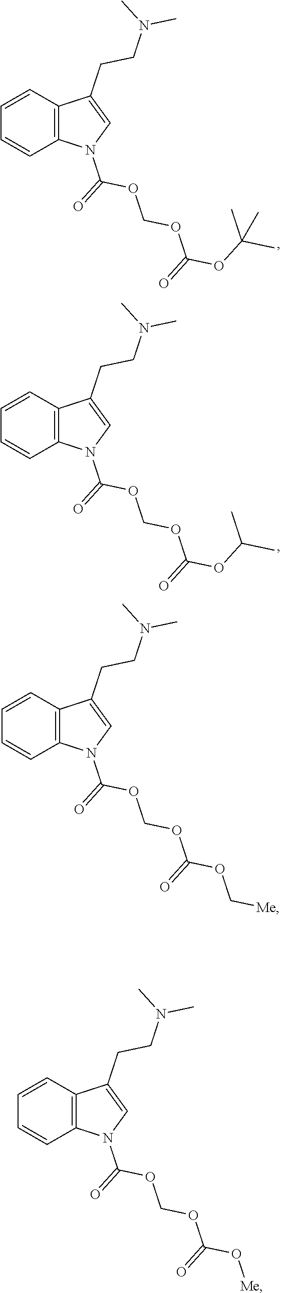

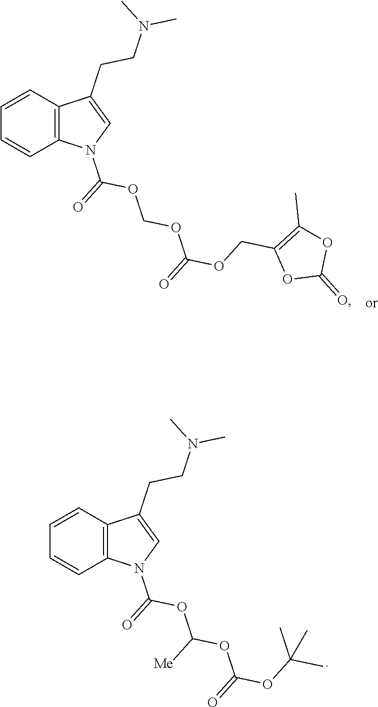 [SMILES: Cc1oc(=O)oc1COC(=O)OCOC(=O)n1cc(CCN(C)C)c2ccccc21 ; CC(OC(=O)OC(C)(C)C)OC(=O)n1cc(CCN(C)C)c2ccccc21]

In some embodiments is a compound of Formula (I) or (Ir) having the structure of Formula (Ir1), or a pharmaceutically acceptable salt thereof:

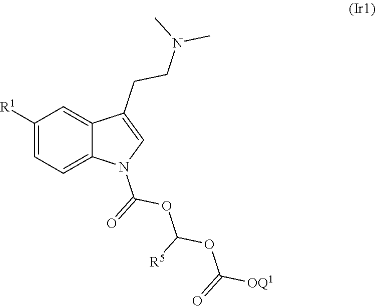

wherein:
R¹ is methoxy or hydrogen;
R⁵ is hydrogen, alkyl, or cycloalkyl, wherein each of alkyl and cycloalkyl is unsubstituted or substituted with one or more R^{A}; and
Q is

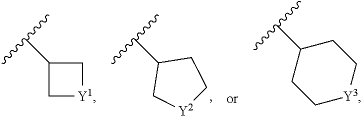

wherein
each of Y¹, Y², or Y³ is independently —O—, —S—, —S(O)—, —S(O)₂—, —N(R^{Y1})—, or —NC(O)R^{Y2}, wherein each of R^{Y1} and R^{Y2} is independently hydrogen, alkyl, heteroalkyl, or heteroaryl.

In some embodiments is a compound of Formula (Ir1), or a pharmaceutically acceptable salt or solvate thereof, wherein each of Y¹, Y², or Y³ is —N(R^{Y1})—. In some embodiments is a compound of Formula (Ir1), or a pharmaceutically acceptable salt or solvate thereof, wherein each of Y¹, Y², or Y³ is —N(R^{Y1})—, wherein R^{Y1} is hydrogen. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently methyl, ethyl, n-propyl, isopropyl, n-butyl, tert-butyl, CH(Et)₂, CH₂CH₂OMe, CH₂CH₂SO₂Me, or CH₂CF₃. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is phenyl. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is benzyl. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently 2-pyridyl, 3-pyridyl, or 4-pyridyl.

In some embodiments is a compound of Formula (I) or (Ir), or a pharmaceutically acceptable salt or solvate thereof, wherein the compound is:

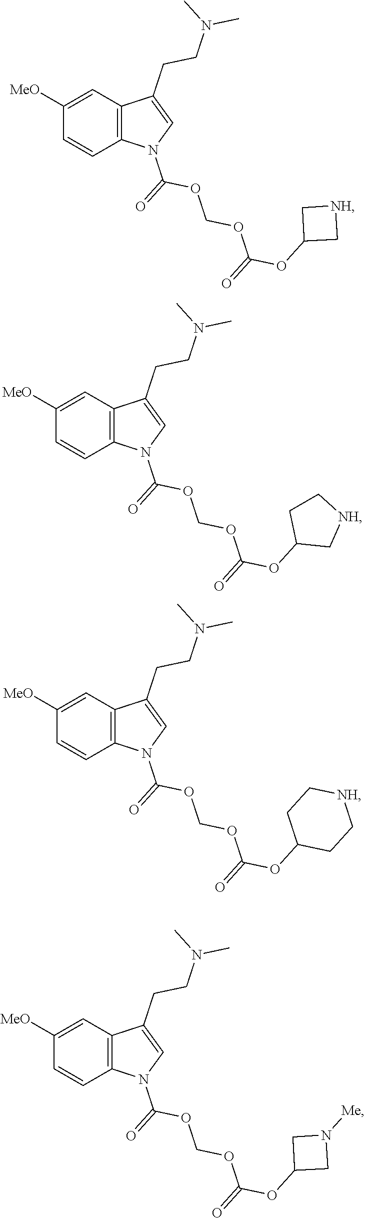

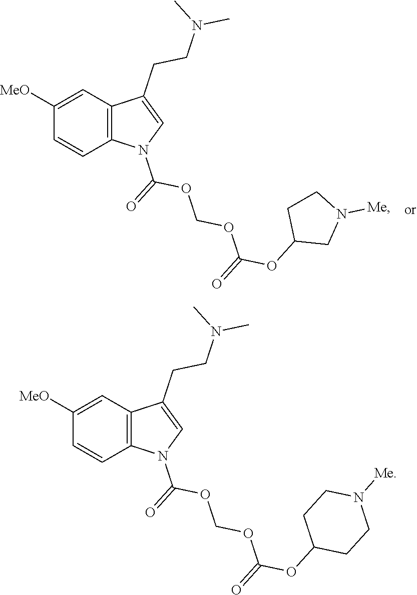

In some embodiments is a compound of Formula (I) or (Ir), or a pharmaceutically acceptable salt or solvate thereof, wherein the compound is:

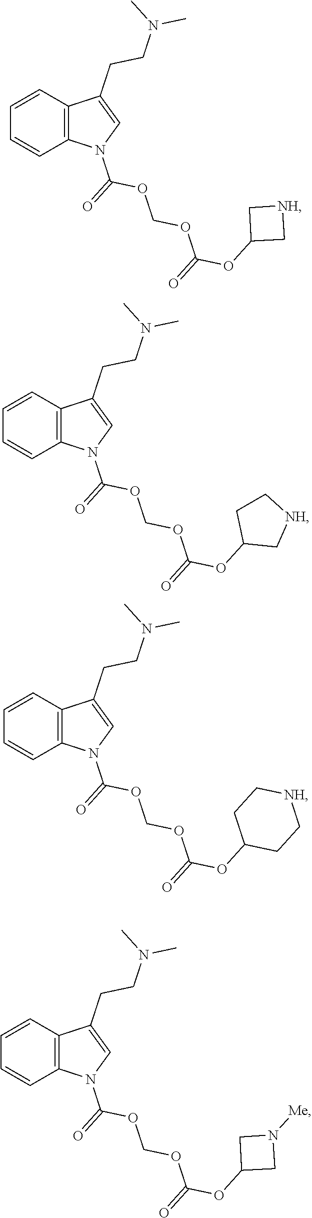

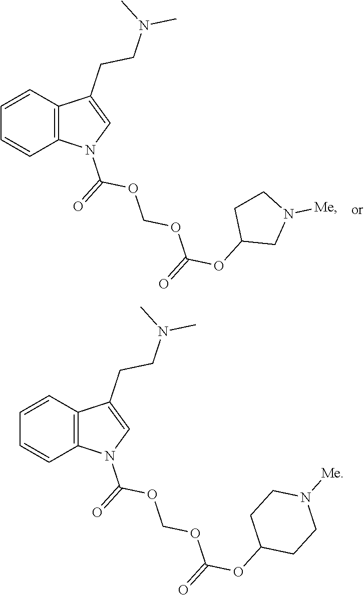

In some embodiments is a compound of Formula (I) or (Ir), or a pharmaceutically acceptable salt or solvate thereof, wherein the compound is:

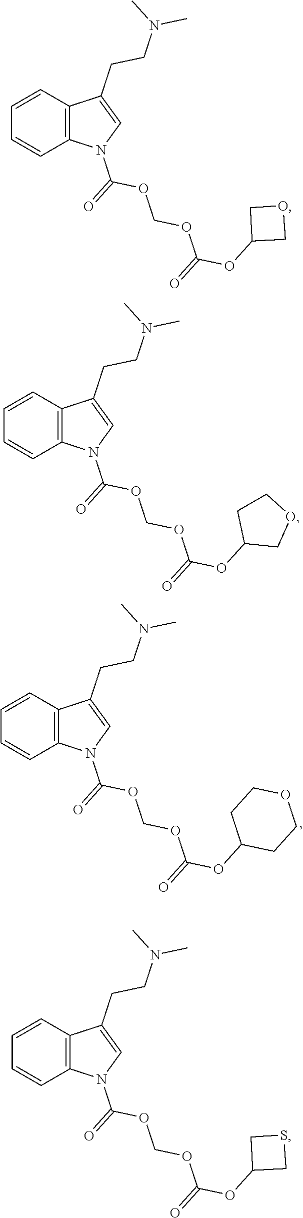

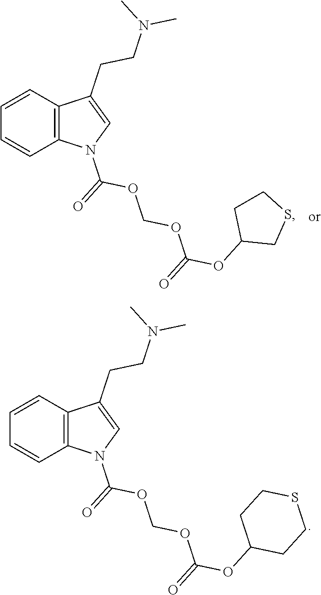

In some embodiments is a compound of Formula (I) or (Ir), or a pharmaceutically acceptable salt or solvate thereof, wherein the compound is:

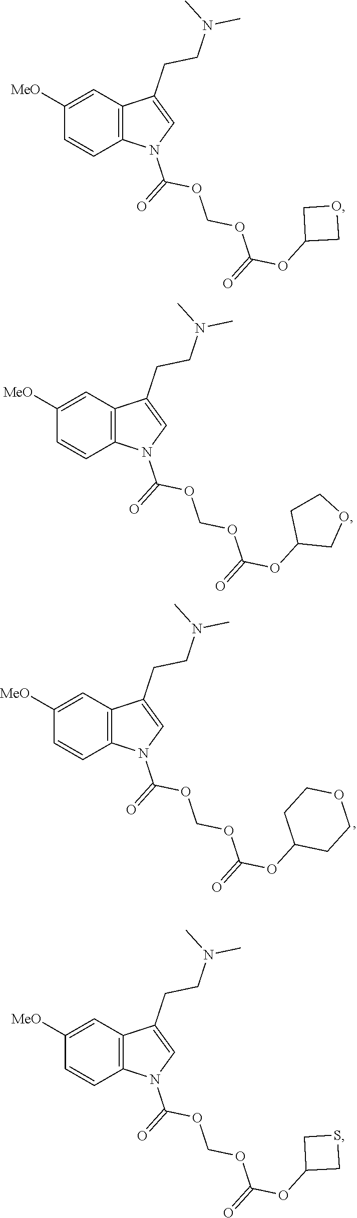

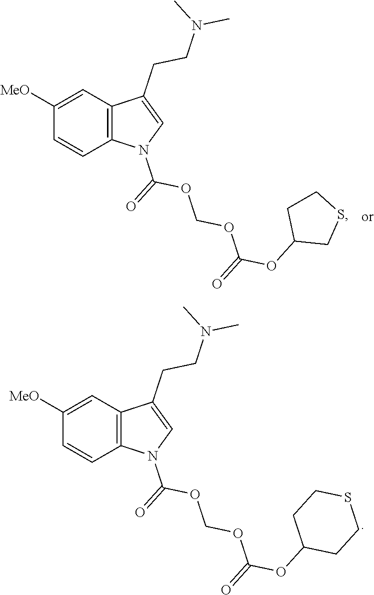

In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

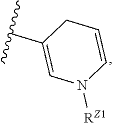

wherein R^{Z1} is hydrogen or alkyl. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

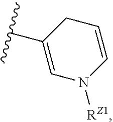

R^{Z1}, wherein R^{Z1} is methyl, ethyl, n-propyl, isopropyl, n-butyl, tert-butyl, or CH(Et)₂. In some embodiments is a compound of Formula (Ir1), or a pharmaceutically acceptable salt or solvate thereof, wherein each of Y¹, Y², or Y³ is —N(R^{Y1})— or —NC(O)R^{Y2}, wherein each of R^{Y1} and R^{Y2} is independently

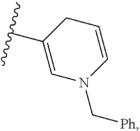 [SMILES: *C1=CN(Cc2ccccc2)C=CC1]

In some embodiments is a compound of Formula (Ir1), or a pharmaceutically acceptable salt or solvate thereof, wherein the compound is:

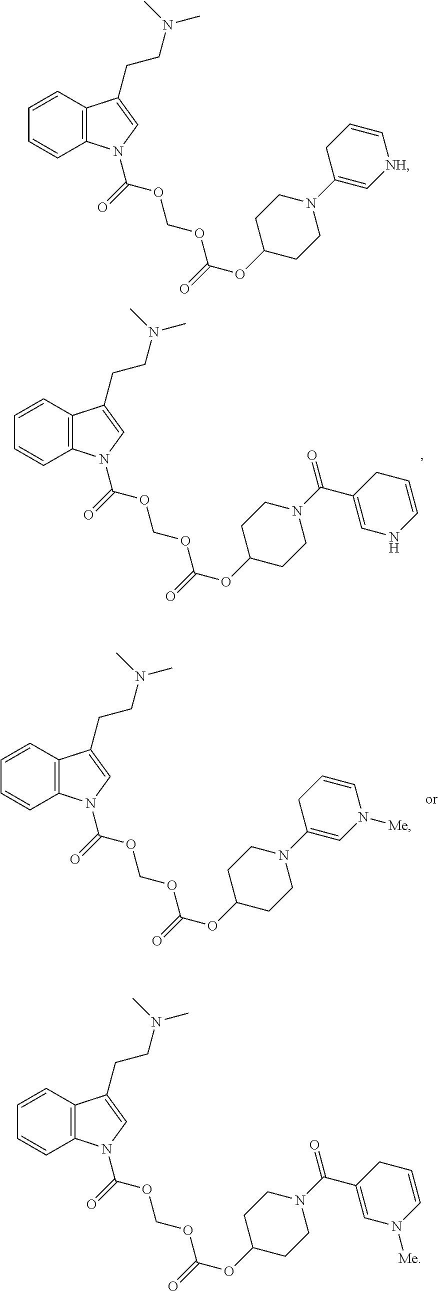

In some embodiments is a compound of Formula (Ir1), or a pharmaceutically acceptable salt or solvate thereof, wherein the compound is:

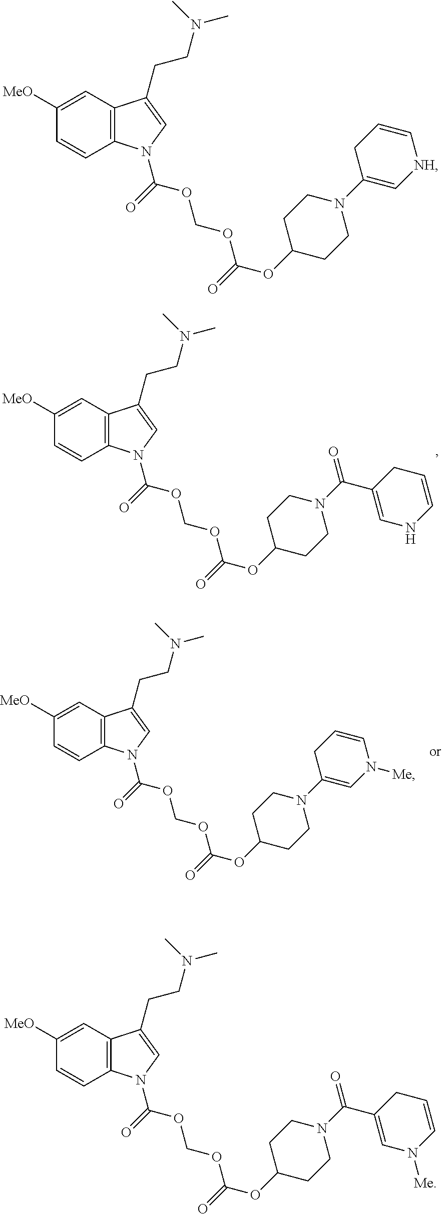

In some embodiments is a compound of Formula (I) having the structure of Formula (Is), or a pharmaceutically acceptable salt thereof:

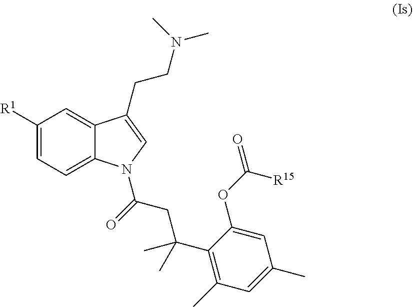

wherein R¹ is hydrogen or methoxy, and R¹⁵ is alkyl, heteroalkyl, cycloalkyl, aryl, or heteroaryl, each of which is unsubstituted or substituted with one or more R^{B}.

In some embodiments is a compound of Formula (I) or (Is), or a pharmaceutically acceptable salt or solvate thereof, wherein R¹⁵ is alkyl. In some embodiments is a compound of Formula (I) or (Is), or a pharmaceutically acceptable salt or solvate thereof, wherein R¹⁵ is unsubstituted alkyl. In some embodiments is a compound of Formula (I) or (Is), or a pharmaceutically acceptable salt or solvate thereof, wherein R¹⁵ is methyl, ethyl, n-propyl, isopropyl, n-butyl, or tert-butyl. In some embodiments is a compound of Formula (I) or (Is), or a pharmaceutically acceptable salt or solvate thereof, wherein R¹⁵ is cycloalkyl. In some embodiments is a compound of Formula (I) or (Is), or a pharmaceutically acceptable salt or solvate thereof, wherein R¹⁵ is cyclopropyl. In some embodiments is a compound of Formula (I) or (Is), or a pharmaceutically acceptable salt or solvate thereof, wherein R¹⁵ is heteroalkyl. In some embodiments is a compound of Formula (I) or (Is), or a pharmaceutically acceptable salt or solvate thereof, wherein R¹⁵ is —CH[CH(Me)₂]NH₂. In some embodiments is a compound of Formula (I) or (Is), or a pharmaceutically acceptable salt or solvate thereof, wherein R¹⁵ is —(CH₂)_{q}CO₂H, wherein q is 1, 2, 3, 4, 5, or 6. In some embodiments is a compound of Formula (I) or (Is), or a pharmaceutically acceptable salt or solvate thereof, wherein R¹⁵ is phenyl. In some embodiments is a compound of Formula (I) or (Is), or a pharmaceutically acceptable salt or solvate thereof, wherein R¹⁵ is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (Is), or a pharmaceutically acceptable salt or solvate thereof, wherein R¹⁵ is methyl, ethyl, isopropyl, or tert-butyl. In some embodiments is a compound of Formula (I) or (Is), or a pharmaceutically acceptable salt or solvate thereof, wherein R¹⁵ is methyl. In some embodiments is a compound of Formula (I) or (Is), or a pharmaceutically acceptable salt or solvate thereof, wherein R¹ is hydrogen, and R¹⁵ is methyl. In some embodiments is a compound of Formula (I) or (Is), or a pharmaceutically acceptable salt or solvate thereof, wherein R¹ is methoxy, and R¹⁵ is methyl.

In some embodiments is a compound of Formula (I) or (Is), or a pharmaceutically acceptable salt or solvate thereof, wherein the compound is:

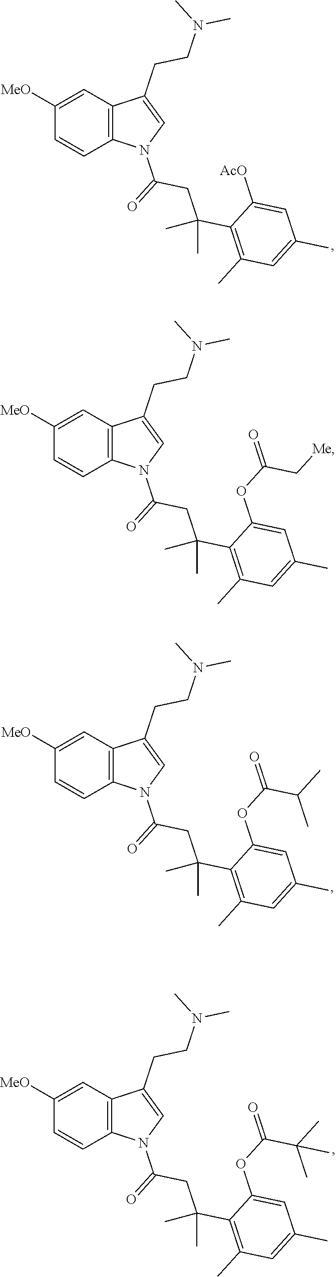 [SMILES: COc1ccc2c(c1)c(CCN(C)C)cn2C(=O)CC(C)(C)c1c(C)cc(C)cc1OC(C)=O ; CCC(=O)Oc1cc(C)cc(C)c1C(C)(C)CC(=O)n1cc(CCN(C)C)c2cc(OC)ccc21 ; COc1ccc2c(c1)c(CCN(C)C)cn2C(=O)CC(C)(C)c1c(C)cc(C)cc1OC(=O)C(C)C ; COc1ccc2c(c1)c(CCN(C)C)cn2C(=O)CC(C)(C)c1c(C)cc(C)cc1OC(=O)C(C)(C)C]

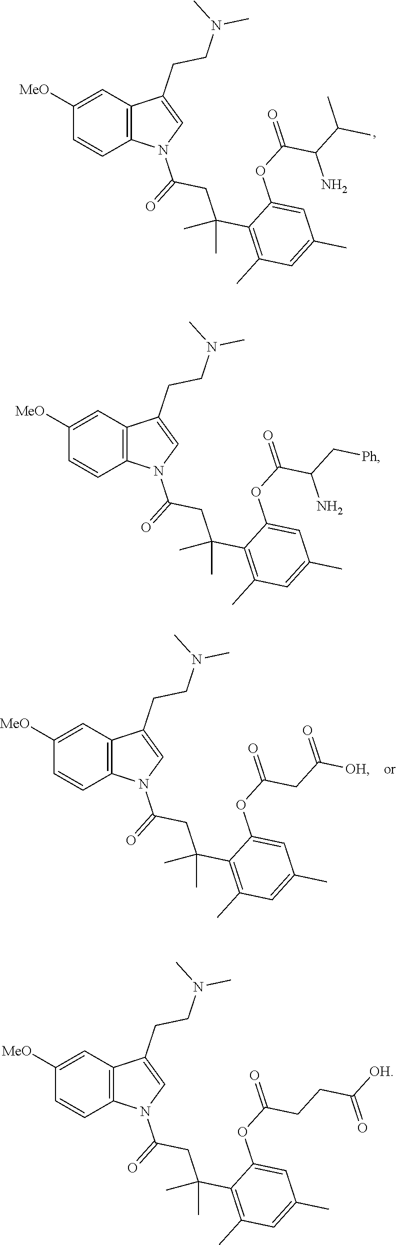 [SMILES: COc1ccc2c(c1)c(CCN(C)C)cn2C(=O)CC(C)(C)c1c(C)cc(C)cc1OC(=O)C(N)C(C)C ; COc1ccc2c(c1)c(CCN(C)C)cn2C(=O)CC(C)(C)c1c(C)cc(C)cc1OC(=O)C(N)Cc1ccccc1 ; COc1ccc2c(c1)c(CCN(C)C)cn2C(=O)CC(C)(C)c1c(C)cc(C)cc1OC(=O)CC(=O)O ; COc1ccc2c(c1)c(CCN(C)C)cn2C(=O)CC(C)(C)c1c(C)cc(C)cc1OC(=O)CCC(=O)O]

In some embodiments is a compound of Formula (I) or (Is), or a pharmaceutically acceptable salt or solvate thereof, wherein the compound is:

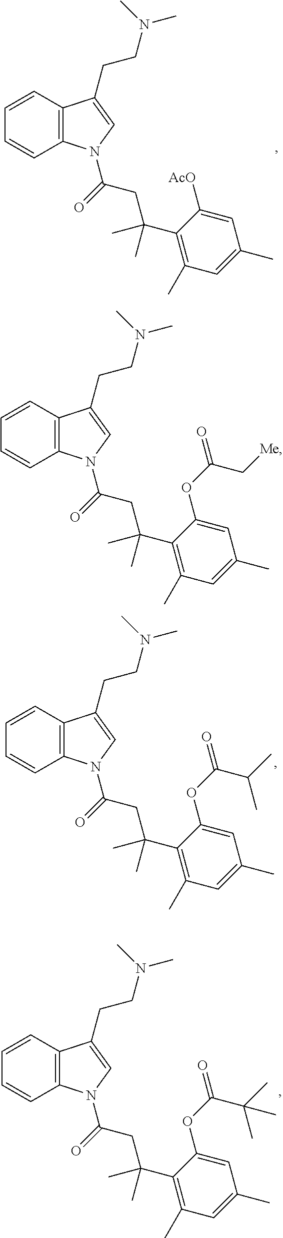

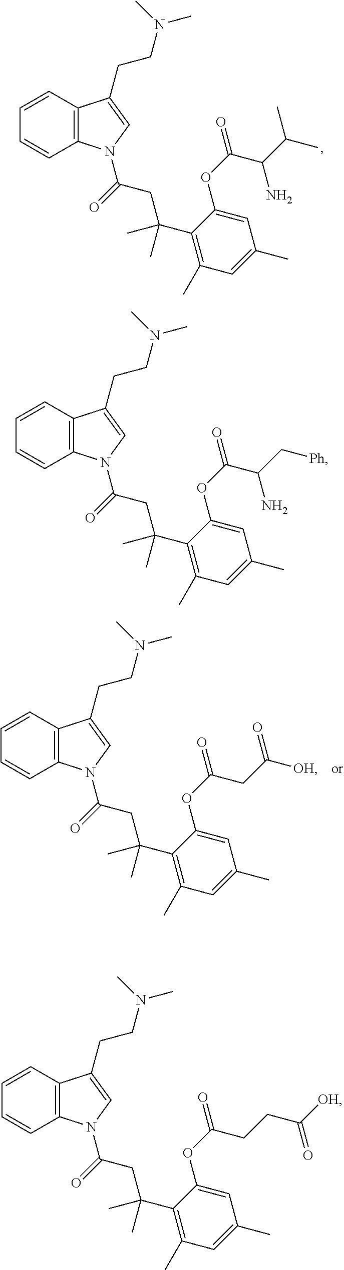

In some embodiments is a compound of Formula (I) having the structure of Formula (It), or a pharmaceutically acceptable salt thereof:

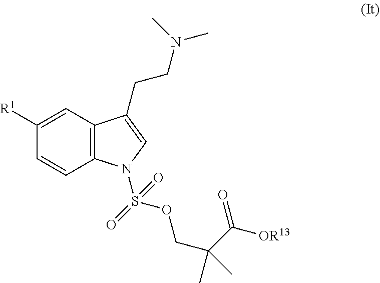

wherein R is hydrogen or met oxy, and R¹³ is alkyl, cycloalkyl, heterocyclylalkyl, aryl, or heteroaryl, each of which is unsubstituted or substituted with one or more R^{B}.

In some embodiments is a compound of Formula (I) or (It), or a pharmaceutically acceptable salt or solvate thereof, wherein R¹³ is alkyl. In some embodiments is a compound of Formula (I) or (It), or a pharmaceutically acceptable salt or solvate thereof, wherein R¹³ is unsubstituted alkyl. In some embodiments is a compound of Formula (I) or (It), or a pharmaceutically acceptable salt or solvate thereof, wherein R¹³ is methyl, ethyl, isopropyl, tert-butyl, n-butyl, n-pentyl, n-hexyl, n-heptyl, or n-octyl. In some embodiments is a compound of Formula (I) or (It), or a pharmaceutically acceptable salt or solvate thereof, wherein R¹³ is cycloalkyl. In some embodiments is a compound of Formula (I) or (It), or a pharmaceutically acceptable salt or solvate thereof, wherein R¹³ is cyclopropyl, cyclobutyl, cyclopentyl, cyclohexyl, or cycloheptyl. In some embodiments is a compound of Formula (I) or (It), or a pharmaceutically acceptable salt or solvate thereof, wherein R¹³ is heteroalkyl. In some embodiments is a compound of Formula (I) or (It), or a pharmaceutically acceptable salt or solvate thereof, wherein R¹³ is —CH₂CH₂OMe, CH₂CH₂SO₂Me, or CH₂CH₂NMe₂. In some embodiments is a compound of Formula (I) or (It), or a pharmaceutically acceptable salt or solvate thereof, wherein R¹³ is (CH₂)ᵤCO₂H, wherein u is 1, 2, 3, 4, 5, or 6. In some embodiments is a compound of Formula (I) or (It), or a pharmaceutically acceptable salt or solvate thereof, wherein R¹³ is aryl. In some embodiments is a compound of Formula (I) or (It), or a pharmaceutically acceptable salt or solvate thereof, wherein R¹³ is phenyl. In some embodiments is a compound of Formula (I) or (It), or a pharmaceutically acceptable salt or solvate thereof, wherein R¹³ is heteroaryl. In some embodiments is a compound of Formula (I) or (It), or a pharmaceutically acceptable salt or solvate thereof, wherein R¹³ is 2-pyridyl, 3-pyridyl, 4-pyridyl, 2-pyrimidyl, 4-pyrimidyl, 5-pyrimidyl, or 6-pyrimidyl. In some embodiments is a compound of Formula (I) or (It), or a pharmaceutically acceptable salt or solvate thereof, wherein R¹³ is heterocyclylalkyl. In some embodiments is a compound of Formula (I) or (It), or a pharmaceutically acceptable salt or solvate thereof, wherein R¹³ is oxetan-3-yl or azetidine-3-yl. In some embodiments is a compound of Formula (I) or (It), or a pharmaceutically acceptable salt or solvate thereof, wherein R¹³ is

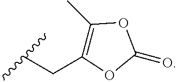 [SMILES: *Cc1oc(=O)oc1C]

In some embodiments is a compound of Formula (I) or (It), or a pharmaceutically acceptable salt or solvate thereof, wherein R¹³ is

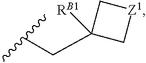

wherein R^{B1} is hydrogen or alkyl, and Z¹ is —O—, —S—, —S(O)—, —S(O)₂—, or —N(R^{C1})—, wherein R^{C1} is hydrogen, alkyl, acetyl, or benzoyl. In some embodiments is a compound of Formula (I) or (It), or a pharmaceutically acceptable salt or solvate thereof, wherein R¹³ is

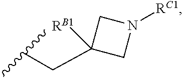

wherein R^{C1} is alkyl. In some embodiments is a compound of Formula (I) or (It), or a pharmaceutically acceptable salt or solvate thereof, wherein R¹³ is

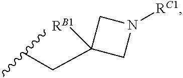

wherein R^{C1} is methyl, acetyl, or benzoyl. In some embodiments is a compound of Formula (I) or (It), or a pharmaceutically acceptable salt or solvate thereof, wherein R¹³ is

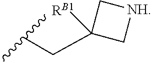

In some embodiments is a compound of Formula (I) or (It), or a pharmaceutically acceptable salt or solvate thereof, wherein R¹³ is

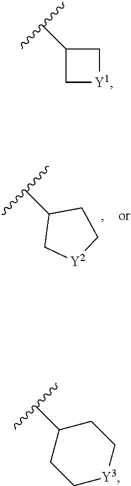

wherein each of Y¹, Y², or Y³ is independently —O—, —S—, —S(O)—, —S(O)₂—, or —N(R^{B2})—, wherein each R^{B2} is independently hydrogen, alkyl, acetyl, or benzoyl. In some embodiments is a compound of Formula (I) or (It), or a pharmaceutically acceptable salt or solvate thereof, wherein R¹³ is

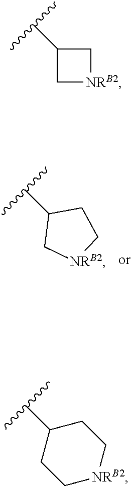

wherein R^{B2} is alkyl. In some embodiments is a compound of Formula (I) or (It), or a pharmaceutically acceptable salt or solvate thereof, wherein R¹³ is

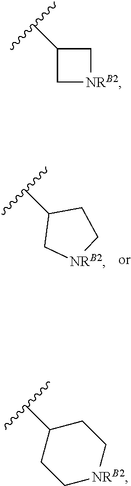

wherein R^{B2} is unsubstituted alkyl. In some embodiments is a compound of Formula (I) or (It), or a pharmaceutically acceptable salt or solvate thereof, wherein R¹³ is

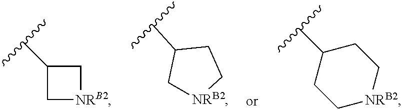

wherein each R^{B2} independently is methyl, acetyl, or benzoyl. In some embodiments is a compound of Formula (I) or (It), or a pharmaceutically acceptable salt or solvate thereof, wherein R¹³ is

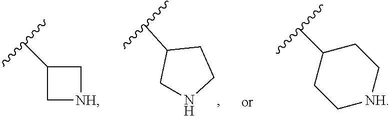 [SMILES: *C1CNC1 ; *C1CCNC1 ; *C1CCNCC1]

In some embodiments is a compound of Formula (I) or (It), or a pharmaceutically acceptable salt or solvate thereof, wherein R¹³ is —CH₂CH₂R^{B3}, wherein R^{B3} is heteroaryl or heterocyclylalkyl. In some embodiments is a compound of Formula (I) or (It), or a pharmaceutically acceptable salt or solvate thereof, wherein R¹³ is —CH₂CH₂R^{B3}, wherein R^{B3} is heterocyclylalkyl. In some embodiments is a compound of Formula (I) or (It), or a pharmaceutically acceptable salt or solvate thereof, wherein R¹³ is —CH₂CH₂R^{B3}, wherein R^{B3} is

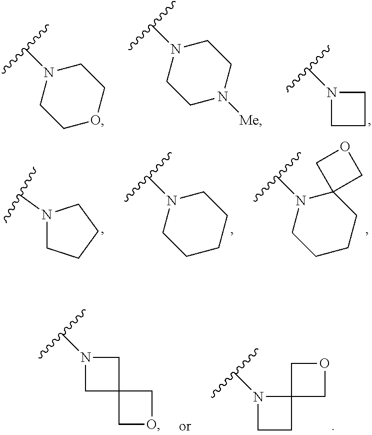

In some embodiments is a compound of Formula (I) or (It), or a pharmaceutically acceptable salt or solvate thereof, wherein the compound is:

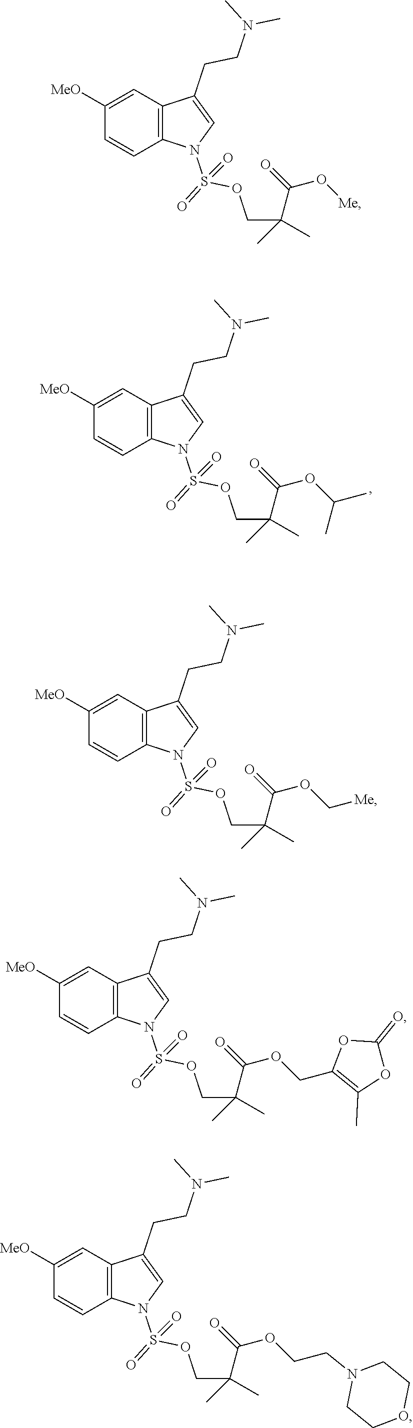 [SMILES: COC(=O)C(C)(C)COS(=O)(=O)n1cc(CCN(C)C)c2cc(OC)ccc21 ; COc1ccc2c(c1)c(CCN(C)C)cn2S(=O)(=O)OCC(C)(C)C(=O)OC(C)C ; CCOC(=O)C(C)(C)COS(=O)(=O)n1cc(CCN(C)C)c2cc(OC)ccc21 ; COc1ccc2c(c1)c(CCN(C)C)cn2S(=O)(=O)OCC(C)(C)C(=O)OCc1oc(=O)oc1C ; COc1ccc2c(c1)c(CCN(C)C)cn2S(=O)(=O)OCC(C)(C)C(=O)OCCN1CCOCC1]

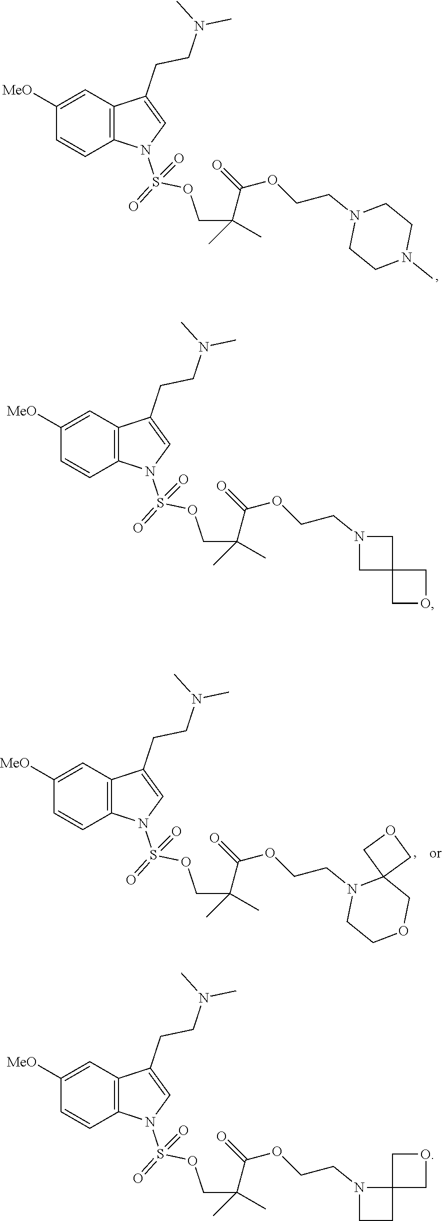 [SMILES: COc1ccc2c(c1)c(CCN(C)C)cn2S(=O)(=O)OCC(C)(C)C(=O)OCCN1CCN(C)CC1 ; COc1ccc2c(c1)c(CCN(C)C)cn2S(=O)(=O)OCC(C)(C)C(=O)OCCN1CC2(COC2)C1 ; COc1ccc2c(c1)c(CCN(C)C)cn2S(=O)(=O)OCC(C)(C)C(=O)OCCN1CCOCC12COC2 ; COc1ccc2c(c1)c(CCN(C)C)cn2S(=O)(=O)OCC(C)(C)C(=O)OCCN1CCC12COC2]

In some embodiments is a compound of Formula (I) or (It), or a pharmaceutically acceptable salt or solvate thereof, wherein the compound is:

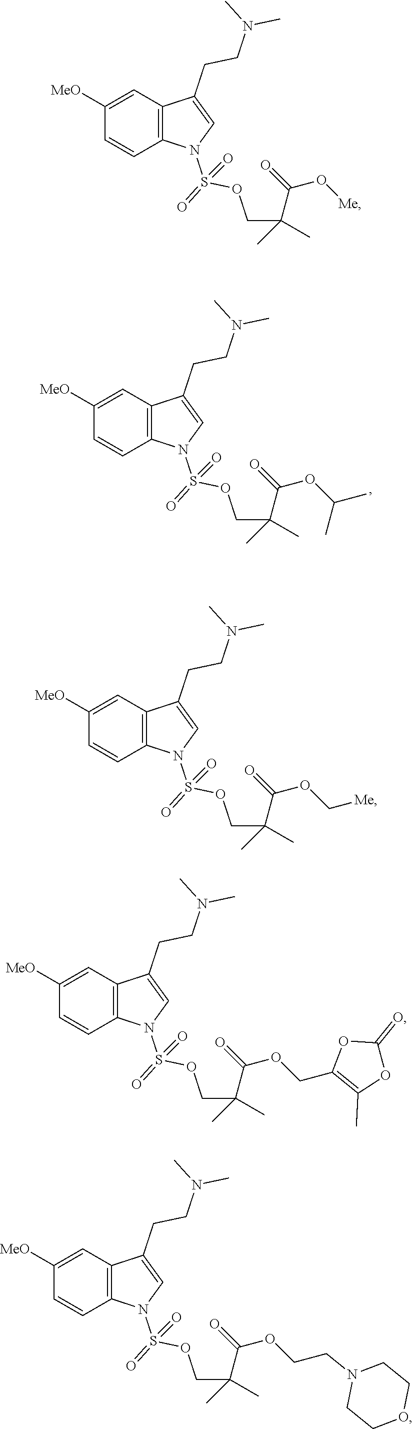 [SMILES: COC(=O)C(C)(C)COS(=O)(=O)n1cc(CCN(C)C)c2cc(OC)ccc21 ; COc1ccc2c(c1)c(CCN(C)C)cn2S(=O)(=O)OCC(C)(C)C(=O)OC(C)C ; CCOC(=O)C(C)(C)COS(=O)(=O)n1cc(CCN(C)C)c2cc(OC)ccc21 ; COc1ccc2c(c1)c(CCN(C)C)cn2S(=O)(=O)OCC(C)(C)C(=O)OCc1oc(=O)oc1C ; COc1ccc2c(c1)c(CCN(C)C)cn2S(=O)(=O)OCC(C)(C)C(=O)OCCN1CCOCC1]

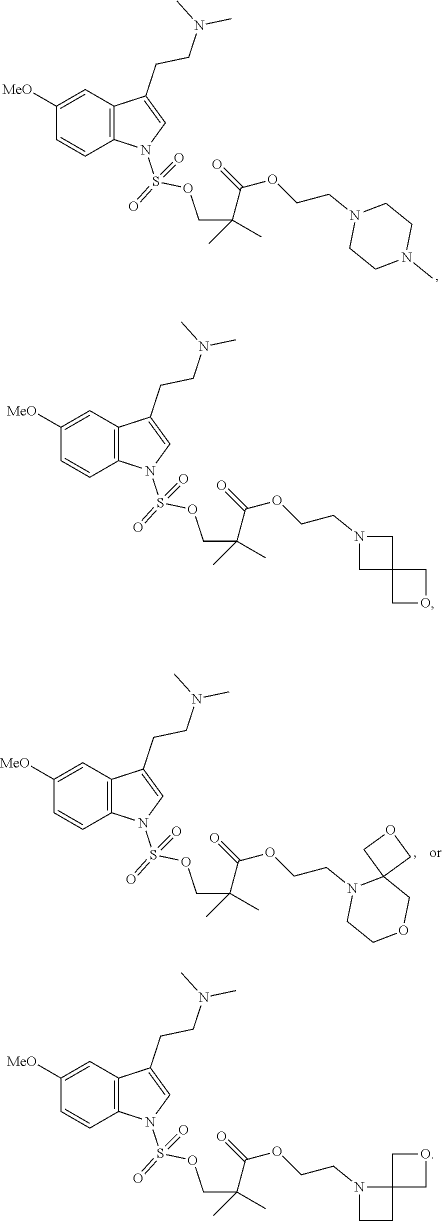 [SMILES: COc1ccc2c(c1)c(CCN(C)C)cn2S(=O)(=O)OCC(C)(C)C(=O)OCCN1CCN(C)CC1 ; COc1ccc2c(c1)c(CCN(C)C)cn2S(=O)(=O)OCC(C)(C)C(=O)OCCN1CC2(COC2)C1 ; COc1ccc2c(c1)c(CCN(C)C)cn2S(=O)(=O)OCC(C)(C)C(=O)OCCN1CCOCC12COC2 ; COc1ccc2c(c1)c(CCN(C)C)cn2S(=O)(=O)OCC(C)(C)C(=O)OCCN1CCC12COC2]

In some embodiments is a compound of Formula (I) or (It), or a pharmaceutically acceptable salt or solvate thereof, wherein the compound is:

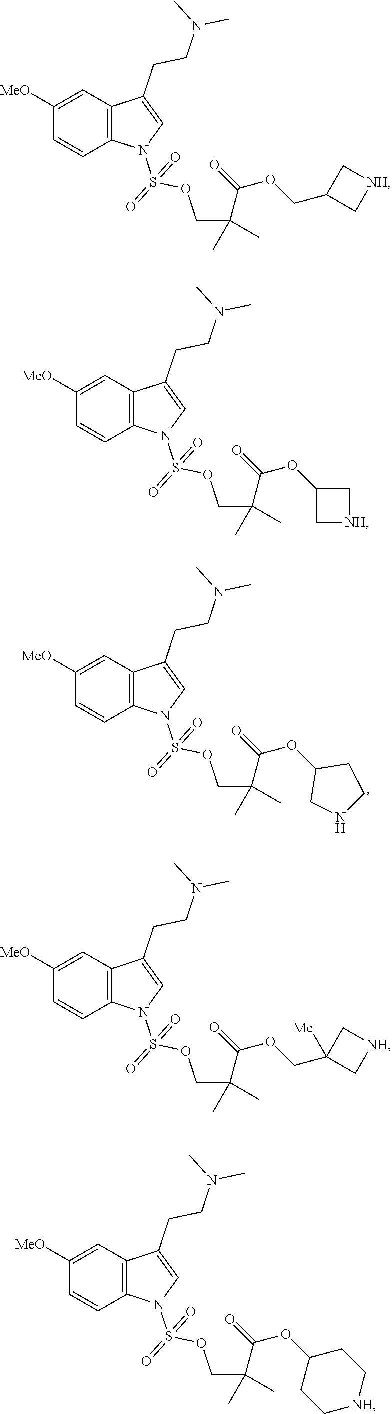

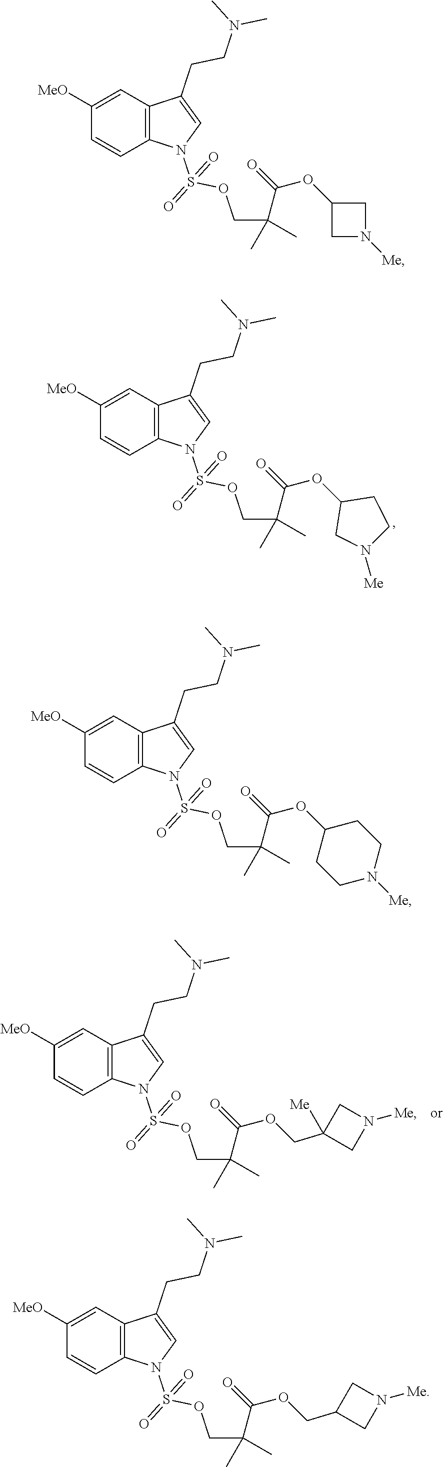

In some embodiments is a compound of Formula (I) or (It), or a pharmaceutically acceptable salt or solvate thereof, wherein the compound is:

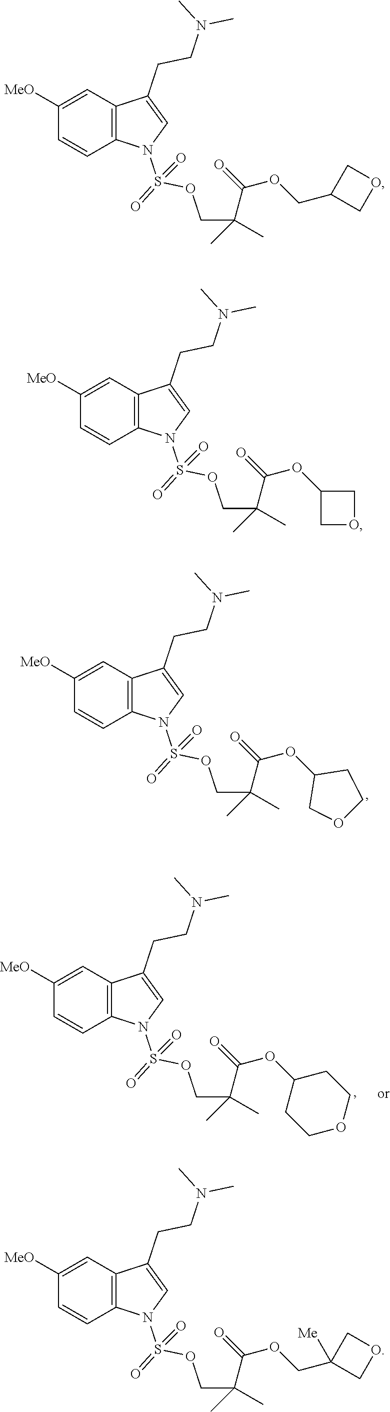

In some embodiments is a compound of Formula (I) or (It), or a pharmaceutically acceptable salt or solvate thereof, wherein the compound is:

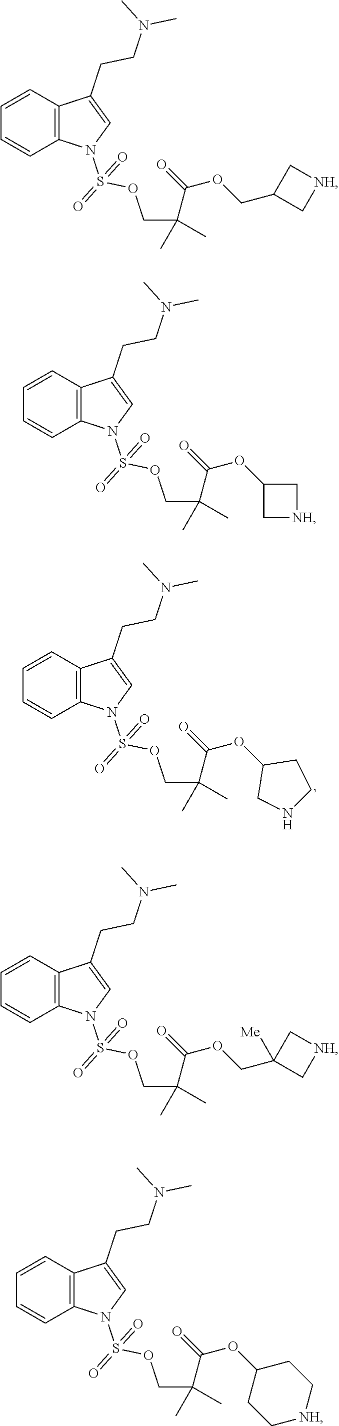

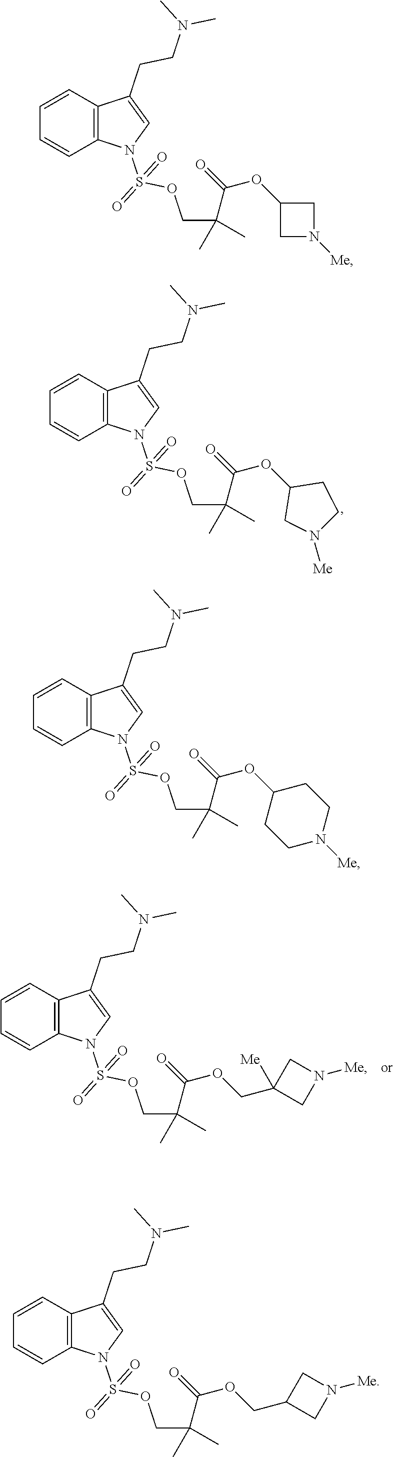

In some embodiments is a compound of Formula (I) or (It), or a pharmaceutically acceptable salt or solvate thereof, wherein the compound is:

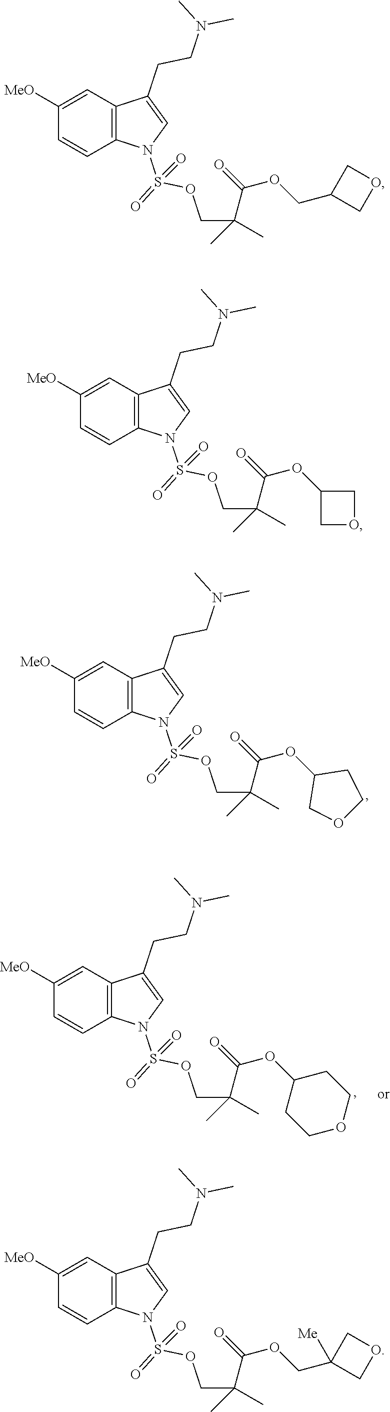

In some embodiments is a compound of Formula (I) having the structure of Formula (Iu), or a pharmaceutically acceptable salt thereof:

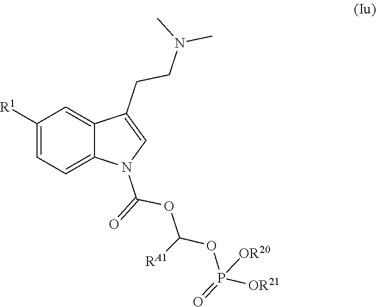

wherein:
R¹ is hydrogen or methoxy;
R^{A1} is hydrogen, alkyl, or cycloalkyl, wherein each of alkyl and cycloalkyl is unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; and
each of R²⁰ and R²¹ is independently hydrogen, alkyl, cycloalkyl, aryl, heterocyclylalkyl, or heteroaryl, wherein each of alkyl, cycloalkyl, aryl, heterocyclylalkyl, and heteroaryl is independently unsubstituted or substituted with one or more R^{B}, or R²⁰ and R²¹ together with the atoms to which they are attached form a heterocyclylalkyl ring that is unsubstituted or substituted with one or more R^{B}.

In some embodiments is a compound of Formula (Iu), or a pharmaceutically acceptable salt or solvate thereof, wherein R^{A1} is alkyl. In some embodiments is a compound of Formula (Iu), or a pharmaceutically acceptable salt or solvate thereof, wherein R^{A1} is unsubstituted alkyl. In some embodiments is a compound of Formula (Iu), or a pharmaceutically acceptable salt or solvate thereof, wherein R^{A1} is methyl, ethyl, isopropyl, or tert-butyl. In some embodiments is a compound of Formula (Iu), or a pharmaceutically acceptable salt or solvate thereof, wherein R^{A1} is hydrogen. In some embodiments is a compound of Formula (Iu), or a pharmaceutically acceptable salt or solvate thereof, wherein R^{A1} is methyl. In some embodiments is a compound of Formula (Iu), or a pharmaceutically acceptable salt or solvate thereof, wherein R^{A1} is hydrogen. In some embodiments is a compound of Formula (Iu), or a pharmaceutically acceptable salt or solvate thereof, wherein R^{A1} is methyl, ethyl, isopropyl, —CH(Et)₂, or tert-butyl.

In some embodiments is a compound of Formula (Iu), or a pharmaceutically acceptable salt or solvate thereof, wherein each of R²⁰ and R²¹ is alkyl. In some embodiments is a compound of Formula (Iu), or a pharmaceutically acceptable salt or solvate thereof, wherein each of R²⁰ and R²¹ is independently unsubstituted alkyl. In some embodiments is a compound of Formula (Iu), or a pharmaceutically acceptable salt or solvate thereof, wherein each of R²⁰ and R²¹ is independently methyl, ethyl, n-propyl, isopropyl, tert-butyl, 3-methyl-1-butyl, n-pentyl, or n-hexyl. In some embodiments is a compound of Formula (Iu), or a pharmaceutically acceptable salt or solvate thereof, wherein each of R²⁰ and R²¹ is benzyl. In some embodiments is a compound of Formula (Iu), or a pharmaceutically acceptable salt or solvate thereof, wherein each of R²⁰ and R²¹ is independently

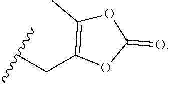 [SMILES: *Cc1oc(=O)oc1C]

In some embodiments is a compound of Formula (Iu), or a pharmaceutically acceptable salt or solvate thereof, wherein each of R²⁰ and R²¹ is phenyl. In some embodiments is a compound of Formula (Iu), or a pharmaceutically acceptable salt or solvate thereof, wherein each of R²⁰ and R²¹ is independently cycloalkyl. In some embodiments is a compound of Formula (Iu), or a pharmaceutically acceptable salt or solvate thereof, wherein each of R²⁰ and R²¹ is independently cyclopropyl, cyclobutyl, cyclopentyl, cyclohexyl, or cycloheptyl. In some embodiments is a compound of Formula (Iu), or a pharmaceutically acceptable salt or solvate thereof, wherein each of R²⁰ and R²¹ is independently heteroaryl. In some embodiments is a compound of Formula (Iu), or a pharmaceutically acceptable salt or solvate thereof, wherein each of R²⁰ and R²¹ is independently 2-pyridyl, 3-pyridyl, 4-pyridyl, 2-pyrimidyl, or 4-pyrimidyl. In some embodiments is a compound of Formula (Iu), or a pharmaceutically acceptable salt or solvate thereof, R²⁰ is hydrogen, and R²¹ is alkyl, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl.

In some embodiments is a compound of Formula (Iu), or a pharmaceutically acceptable salt or solvate thereof, wherein each of R²⁰ and R²¹ is independently alkyl or cycloalkyl. In some embodiments is a compound of Formula (Iu), or a pharmaceutically acceptable salt or solvate thereof, wherein each of R²⁰ and R²¹ is independently unsubstituted alkyl, and R^{A1} is hydrogen. In some embodiments is a compound of Formula (Iu), or a pharmaceutically acceptable salt or solvate thereof, wherein each of R²⁰ and R²¹ is independently unsubstituted alkyl, and R¹ is methyl. In some embodiments is a compound of Formula (Iu), or a pharmaceutically acceptable salt or solvate thereof, wherein each of R²⁰ and R²¹ is tert-butyl, R^{A1} is hydrogen, and R¹ is methoxy. In some embodiments is a compound of Formula (Iu), or a pharmaceutically acceptable salt or solvate thereof, wherein each of R²⁰ and R²¹ is tert-butyl, R^{A1} is hydrogen, and R¹ is hydrogen.

In some embodiments is a compound of Formula (Iu), or a pharmaceutically acceptable salt or solvate thereof, wherein the compound is:

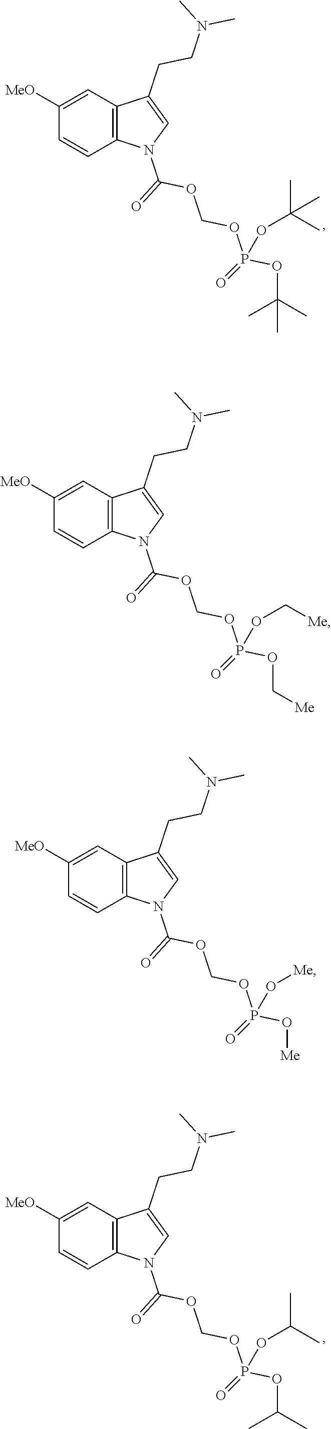 [SMILES: COc1ccc2c(c1)c(CCN(C)C)cn2C(=O)OCOP(=O)(OC(C)(C)C)OC(C)(C)C ; CCOP(=O)(OCC)OCOC(=O)n1cc(CCN(C)C)c2cc(OC)ccc21 ; COc1ccc2c(c1)c(CCN(C)C)cn2C(=O)OCOP(=O)(OC)OC ; COc1ccc2c(c1)c(CCN(C)C)cn2C(=O)OCOP(=O)(OC(C)C)OC(C)C]

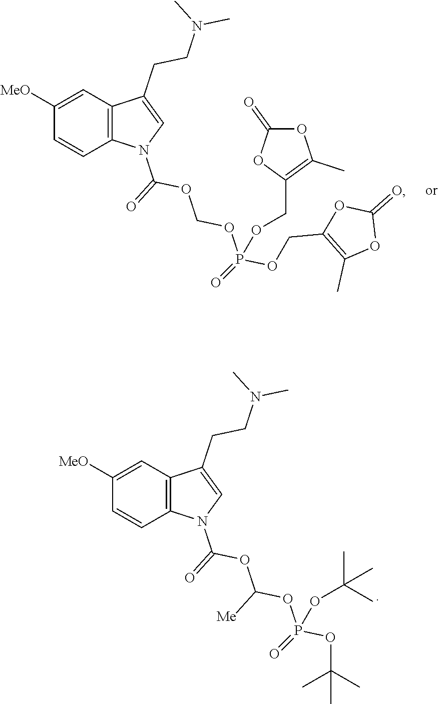 [SMILES: COc1ccc2c(c1)c(CCN(C)C)cn2C(=O)OCOP(=O)(OCc1oc(=O)oc1C)OCc1oc(=O)oc1C ; COc1ccc2c(c1)c(CCN(C)C)cn2C(=O)OC(C)OP(=O)(OC(C)(C)C)OC(C)(C)C]

In some embodiments is a compound of Formula (Iu), or a pharmaceutically acceptable salt or solvate thereof, wherein the compound is:

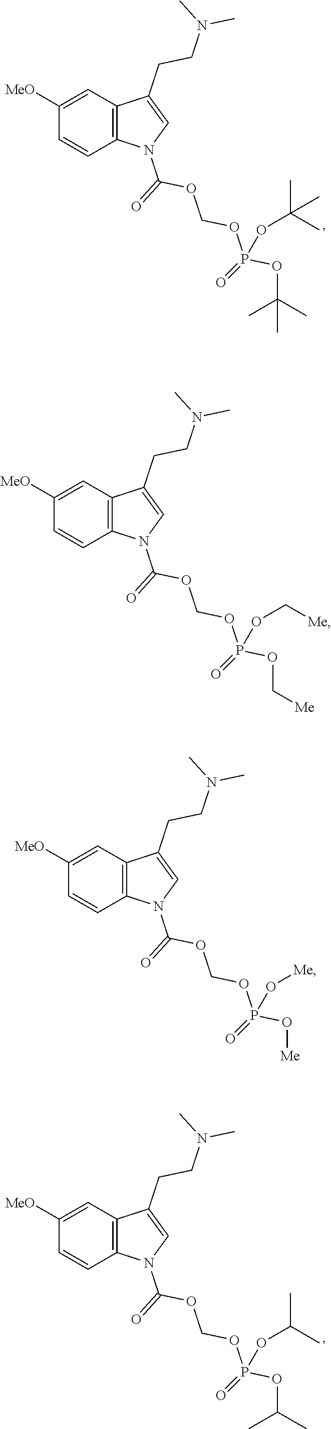 [SMILES: COc1ccc2c(c1)c(CCN(C)C)cn2C(=O)OCOP(=O)(OC(C)(C)C)OC(C)(C)C ; CCOP(=O)(OCC)OCOC(=O)n1cc(CCN(C)C)c2cc(OC)ccc21 ; COc1ccc2c(c1)c(CCN(C)C)cn2C(=O)OCOP(=O)(OC)OC ; COc1ccc2c(c1)c(CCN(C)C)cn2C(=O)OCOP(=O)(OC(C)C)OC(C)C]

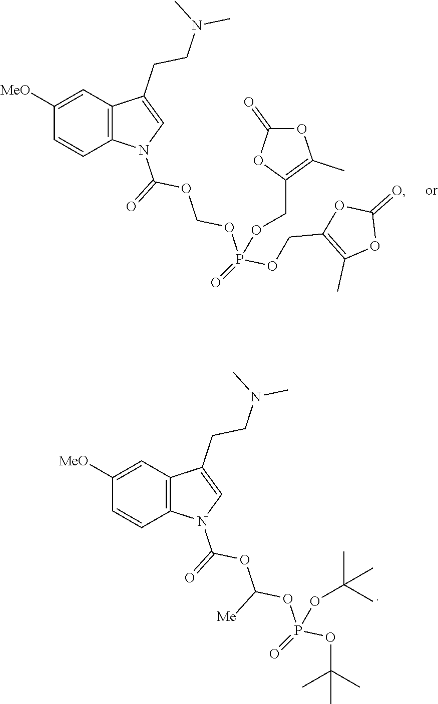 [SMILES: COc1ccc2c(c1)c(CCN(C)C)cn2C(=O)OCOP(=O)(OCc1oc(=O)oc1C)OCc1oc(=O)oc1C ; COc1ccc2c(c1)c(CCN(C)C)cn2C(=O)OC(C)OP(=O)(OC(C)(C)C)OC(C)(C)C]

In some embodiments is a compound of Formula (I) having the structure of Formula (Iv), or a pharmaceutically acceptable salt thereof:

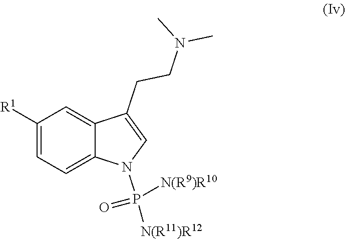

wherein:
R¹ is hydrogen or methoxy;
each of R⁹ and R¹⁰ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R⁹ and R¹⁰ together with the atom to which they are attached form a heterocyclylalkyl ring that is unsubstituted or substituted with one or more R^{A}; and
each of R¹¹ and R¹² is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}, or R¹¹ and R¹² together with the atoms to which they are attached form a heterocyclylalkyl ring that is unsubstituted or substituted with one or more R^{A}.

In some embodiments is a compound of Formula (Iv), or a pharmaceutically acceptable salt or solvate thereof, wherein each of R⁹ and R¹⁰ is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}. In some embodiments is a compound of Formula (Iv), or a pharmaceutically acceptable salt or solvate thereof, wherein each of R¹¹ and R¹² is independently alkyl, heteroalkyl, cycloalkyl, heterocyclylalkyl, aryl, heteroaryl, or hydrogen, wherein each alkyl, heteroalkyl, cycloalkyl, heterocyclylalkyl, aryl, and heteroaryl is independently unsubstituted or substituted with one or more R^{A}. In some embodiments is a compound of Formula (Iv), or a pharmaceutically acceptable salt or solvate thereof, wherein each of R⁹, R¹⁰, R¹¹, and R¹² is independently alkyl, cycloalkyl, or hydrogen, wherein each alkyl and cycloalkyl is independently unsubstituted or substituted with one or more R^{A}. In some embodiments is a compound of Formula (Iv), or a pharmaceutically acceptable salt or solvate thereof, wherein each of R⁹, R¹⁰, R¹¹, and R¹² is independently hydrogen, methyl, ethyl, isopropyl, n-propyl, isobutyl, tert-butyl, or n-butyl. In some embodiments is a compound of Formula (Iv), or a pharmaceutically acceptable salt or solvate thereof, wherein each of R⁹, R¹⁰, R¹¹, and R¹² is methyl.

In some embodiments is a compound of Formula (Iv), or a pharmaceutically acceptable salt or solvate thereof, wherein the compound is:

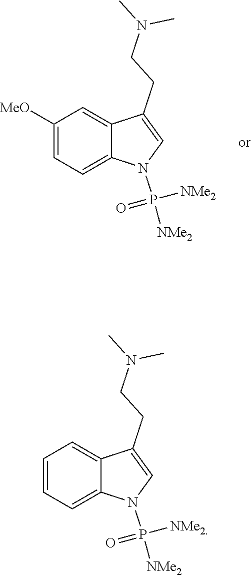 [SMILES: COc1ccc2c(c1)c(CCN(C)C)cn2P(=O)(N(C)C)N(C)C ; CN(C)CCc1cn(P(=O)(N(C)C)N(C)C)c2ccccc12]

In some embodiments, the compound of Formula (I) having the structure of Formula (Iw), or a pharmaceutically acceptable salt thereof:

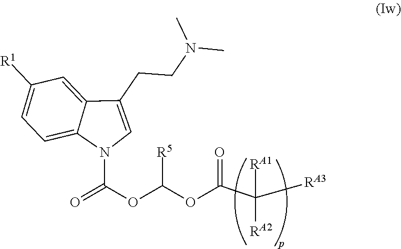

wherein:
R¹ is hydrogen or methoxy;
each R^{A1} and R^{A2} is independently hydrogen, alkyl, or cycloalkyl, wherein each alkyl and cycloalkyl is independently unsubstituted or substituted with one or more alkyl, aryl, halogen, —OR¹³, —NR(R¹⁸)R¹⁹, —C(O)R¹⁴, —OC(O)R¹⁵, —OC(O)OR¹⁶, or —OC(O)N(R¹⁸)R¹⁹; R^{A3} is —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, or —OP(O)OR²⁰(OR²¹), and
p is 1, 2, 3, 4, 5, 6, 7, 8, 9, or 10.

In some embodiments is a compound of Formula (Iw), or a pharmaceutically acceptable salt or solvate thereof, wherein each R^{A1} and R^{A2} is independently hydrogen, alkyl, or cycloalkyl. In some embodiments is a compound of Formula (Iw), or a pharmaceutically acceptable salt or solvate thereof, wherein each R^{A1} and R^{A2} is independently hydrogen, unsubstituted alkyl, or unsubstituted cycloalkyl. In some embodiments is a compound of Formula (Iw), or a pharmaceutically acceptable salt or solvate thereof, wherein each R^{A1} and R^{A2} is independently hydrogen.

In some embodiments is a compound of Formula (Iw), or a pharmaceutically acceptable salt or solvate thereof, wherein R^{A3} is —C(O)OR¹³, —N(R¹³)C(O)OR¹⁴, —N(R¹³)C(O)R¹⁴, or —C(O)R¹⁴. In some embodiments is a compound of Formula (Iw), or a pharmaceutically acceptable salt or solvate thereof, wherein R^{A3} is —C(O)OR¹³. In some embodiments is a compound of Formula (Iw), or a pharmaceutically acceptable salt or solvate thereof, wherein R^{A3} is —C(O)OR¹³, wherein R¹³ is hydrogen or alkyl that is unsubstituted or substituted with one or more R^{B}. In some embodiments is a compound of Formula (Iw), or a pharmaceutically acceptable salt or solvate thereof, wherein R^{A3} is —C(O)OR¹³, wherein R¹³ is hydrogen or alkyl that is unsubstituted. In some embodiments is a compound of Formula (Iw), or a pharmaceutically acceptable salt or solvate thereof, wherein R^{A3} is —C(O)OR¹³, wherein R¹³ is hydrogen, methyl, ethyl, n-propyl, isopropyl, n-butyl, isobutyl, or tert-butyl. In some embodiments is a compound of Formula (Iw), or a pharmaceutically acceptable salt or solvate thereof, wherein R^{A3} is —C(O)OR¹³, wherein R¹³ is hydrogen or tert-butyl.

In some embodiments is a compound of Formula (Iw), or a pharmaceutically acceptable salt or solvate thereof, wherein p is 1, 2, 3, 4, or 5. In some embodiments is a compound of Formula (Iw), or a pharmaceutically acceptable salt or solvate thereof, wherein p is 2, 3, 4, or 5.

In some embodiments is a compound of Formula (Iv), or a pharmaceutically acceptable salt or solvate thereof, wherein the compound is:

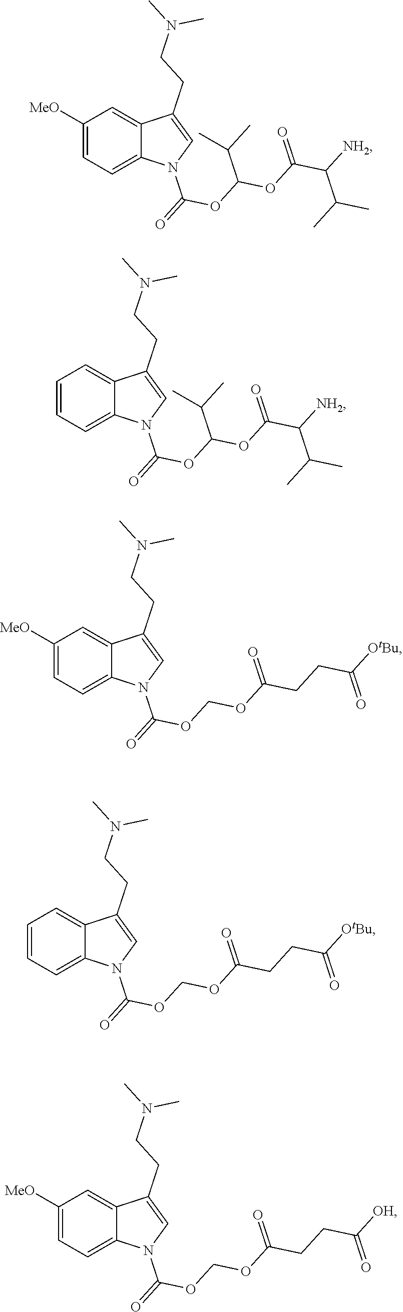

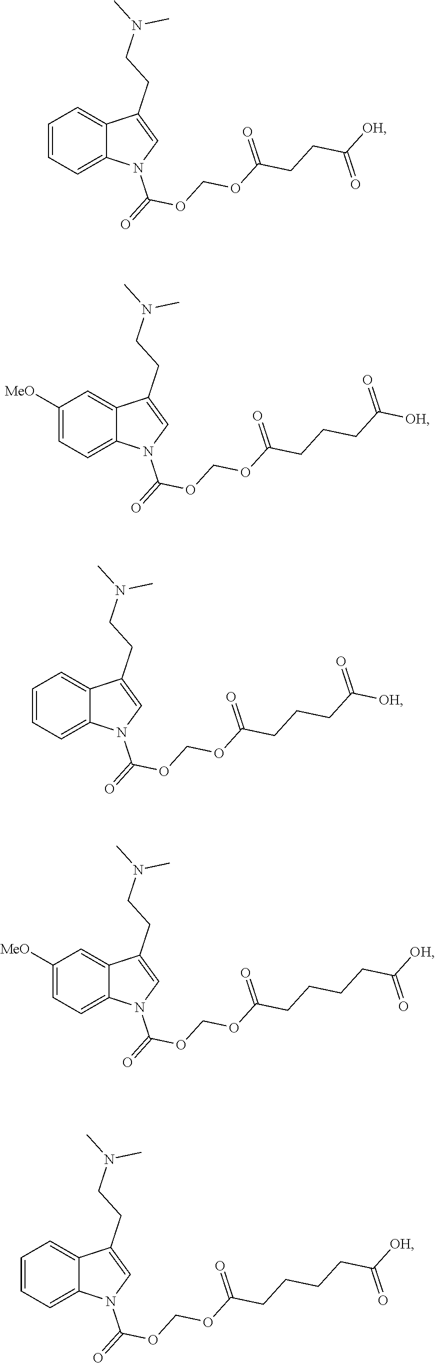 [SMILES: CN(C)CCc1cn(C(=O)OCOC(=O)CCC(=O)O)c2ccccc12 ; COc1ccc2c(c1)c(CCN(C)C)cn2C(=O)OCOC(=O)CCCC(=O)O ; CN(C)CCc1cn(C(=O)OCOC(=O)CCCC(=O)O)c2ccccc12 ; COc1ccc2c(c1)c(CCN(C)C)cn2C(=O)OCOC(=O)CCCCC(=O)O ; CN(C)CCc1cn(C(=O)OCOC(=O)CCCCC(=O)O)c2ccccc12]

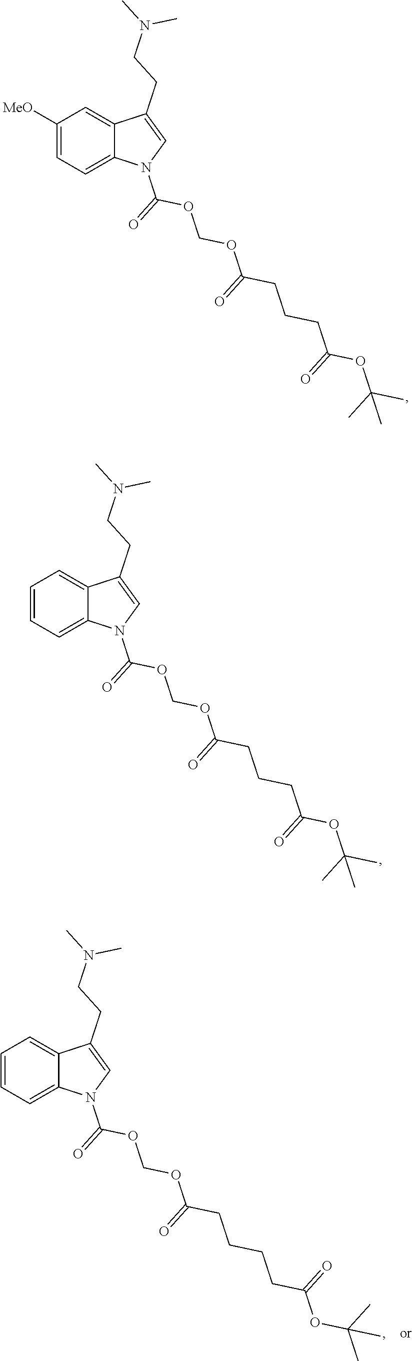 [SMILES: COc1ccc2c(c1)c(CCN(C)C)cn2C(=O)OCOC(=O)CCCC(=O)OC(C)(C)C ; CN(C)CCc1cn(C(=O)OCOC(=O)CCCC(=O)OC(C)(C)C)c2ccccc12 ; CN(C)CCc1cn(C(=O)OCOC(=O)CCCCC(=O)OC(C)(C)C)c2ccccc12]

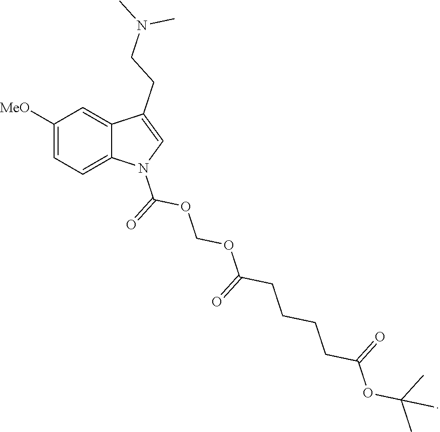 [SMILES: COc1ccc2c(c1)c(CCN(C)C)cn2C(=O)OCOC(=O)CCCCC(=O)OC(C)(C)C]

In some embodiments is a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, wherein R¹ is methoxy. In some embodiments is a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, wherein R¹ is hydrogen.

In some embodiments is a compound of Formula (I), or a pharmaceutically acceptable salt or solvate thereof, wherein R⁴ is hydrogen or alkyl. In some embodiments is a compound of Formula (I), or a pharmaceutically acceptable salt or solvate thereof, wherein R⁴ is alkyl. In some embodiments is a compound of Formula (I), or a pharmaceutically acceptable salt or solvate thereof, wherein R⁴ is hydrogen or unsubstituted alkyl. In some embodiments is a compound of Formula (I), or a pharmaceutically acceptable salt or solvate thereof, wherein R⁴ is hydrogen. In some embodiments is a compound of Formula (I), or a pharmaceutically acceptable salt or solvate thereof, wherein R⁴ is unsubstituted alkyl.

In some embodiments is a compound of Formula (I), or a pharmaceutically acceptable salt or solvate thereof, wherein R⁵ is hydrogen or alkyl. In some embodiments is a compound of Formula (I), or a pharmaceutically acceptable salt or solvate thereof, wherein R⁵ is alkyl. In some embodiments is a compound of Formula (I), or a pharmaceutically acceptable salt or solvate thereof, wherein R⁵ is hydrogen or unsubstituted alkyl. In some embodiments is a compound of Formula (I), or a pharmaceutically acceptable salt or solvate thereof, wherein R⁵ is hydrogen. In some embodiments is a compound of Formula (I), or a pharmaceutically acceptable salt or solvate thereof, wherein R⁵ is unsubstituted alkyl.

In some embodiments is a compound of Formula (I), or a pharmaceutically acceptable salt or solvate thereof, wherein R² is —C(O)OCH(R⁵)OC(O)R⁶. In some embodiments is a compound of Formula (I), or a pharmaceutically acceptable salt or solvate thereof, wherein R² is —C(O)OCH(R⁵)OC(O)R⁶, wherein R⁵ is hydrogen or alkyl. In some embodiments is a compound of Formula (I), or a pharmaceutically acceptable salt or solvate thereof, wherein R² is —C(O)OCH(R⁵)OC(O)R⁶, wherein R⁵ is hydrogen or unsubstituted alkyl. In some embodiments is a compound of Formula (I), or a pharmaceutically acceptable salt or solvate thereof, wherein R² is —C(O)OCH₂OC(O)R⁶. In some embodiments is a compound of Formula (I), or a pharmaceutically acceptable salt or solvate thereof, wherein R² is —C(O)OCH(R⁵)OC(O)R⁶, wherein R⁶ is alkyl, cycloalkyl, heterocyclylalkyl, aryl, or heteroaryl. In some embodiments is a compound of Formula (I), or a pharmaceutically acceptable salt or solvate thereof, wherein R² is —C(O)OCH(R⁵)OC(O)R⁶, wherein R⁶ is alkyl. In some embodiments is a compound of Formula (I), or a pharmaceutically acceptable salt or solvate thereof, wherein R² is —C(O)OCH(R⁵)OC(O)R⁶, wherein R⁶ is heteroalkyl. In some embodiments is a compound of Formula (I), or a pharmaceutically acceptable salt or solvate thereof, wherein R² is —C(O)OCH(R⁵)OC(O)R⁶, wherein R⁶ is unsubstituted alkyl, unsubstituted cycloalkyl, unsubstituted heterocyclylalkyl, unsubstituted aryl, or unsubstituted heteroaryl. In some embodiments is a compound of Formula (I), or a pharmaceutically acceptable salt or solvate thereof, wherein R² is —C(O)OCH(R⁵)OC(O)R⁶, wherein R⁶ is alkyl. In some embodiments is a compound of Formula (I), or a pharmaceutically acceptable salt or solvate thereof, wherein R² is —C(O)OCH(R⁵)OC(O)R⁶, wherein R⁶ is heteroalkyl. In some embodiments is a compound of Formula (I), or a pharmaceutically acceptable salt or solvate thereof, wherein R² is —C(O)OCH(R⁵)OC(O)R⁶, wherein R⁶ is heterocyclylalkyl substituted with arylalkyl.

In some embodiments is a compound of Formula (I), or a pharmaceutically acceptable salt or solvate thereof, wherein R² is —C(O)OCH(R⁵)OC(O)OR⁶. In some embodiments is a compound of Formula (I), or a pharmaceutically acceptable salt or solvate thereof, wherein R² is —C(O)OCH₂OC(O)OR⁶. In some embodiments is a compound of Formula (I), or a pharmaceutically acceptable salt or solvate thereof, wherein R² is —C(O)OCH(R⁵)OC(O)OR⁶, wherein R⁵ is alkyl. In some embodiments is a compound of Formula (I), or a pharmaceutically acceptable salt or solvate thereof, wherein R² is —C(O)OCH(R⁵)OC(O)OR⁶, wherein R⁵ is hydrogen or unsubstituted alkyl. In some embodiments is a compound of Formula (I), or a pharmaceutically acceptable salt or solvate thereof, wherein R² is —C(O)OCH(R⁵)OC(O)OR⁶, wherein R⁶ is heteroalkyl. In some embodiments is a compound of Formula (I), or a pharmaceutically acceptable salt or solvate thereof, wherein R² is —C(O)OCH(R⁵)OC(O)OR⁶, wherein R⁶ is alkyl, heteroalkyl, cycloalkyl, or heterocyclylalkyl. In some embodiments is a compound of Formula (I), or a pharmaceutically acceptable salt or solvate thereof, wherein R² is —C(O)OCH(R⁵)OC(O)OR⁶, wherein R⁶ is heterocyclylalkyl substituted with alkyl, heteroalkyl, or arylalkyl. In some embodiments is a compound of Formula (I), or a pharmaceutically acceptable salt or solvate thereof, wherein R² is —C(O)OCH(R⁵)OC(O)OR⁶, wherein R⁶ is unsubstituted heteroalkyl. In some embodiments is a compound of Formula (I), or a pharmaceutically acceptable salt or solvate thereof, wherein R² is —C(O)OCH(R⁵)OC(O)OR⁶, wherein R⁶ is unsubstituted alkyl, unsubstituted heteroalkyl, unsubstituted cycloalkyl, or unsubstituted heterocyclylalkyl. In some embodiments is a compound of Formula (I), or a pharmaceutically acceptable salt or solvate thereof, wherein R² is —C(O)OCH(R⁵)OC(O)OR⁶, wherein R⁶ is heterocyclylalkyl substituted with alkyl, heteroalkyl, or arylalkyl. In some embodiments is a compound of Formula (I), or a pharmaceutically acceptable salt or solvate thereof, wherein R² is —C(O)OCH(R⁵)OC(O)OR⁶, wherein R⁶ is heterocyclylalkyl that is unsubstituted.

In some embodiments is a compound of Formula (I), or a pharmaceutically acceptable salt or solvate thereof, wherein R² is —C(O)N(R⁹)R¹⁰. In some embodiments is a compound of Formula (I), or a pharmaceutically acceptable salt or solvate thereof, wherein R² is —C(O)N(R⁹)R¹⁰, wherein each of R⁹ and R¹⁰ is independently alkyl. In some embodiments is a compound of Formula (I), or a pharmaceutically acceptable salt or solvate thereof, wherein R² is —C(O)N(R⁹)R¹⁰, wherein each of R⁹ and R¹⁰ is independently alkyl that is unsubstituted. In some embodiments is a compound of Formula (I), or a pharmaceutically acceptable salt or solvate thereof, wherein R² is —C(O)N(H)R¹⁰, wherein R¹⁰ is alkyl. In some embodiments is a compound of Formula (I), or a pharmaceutically acceptable salt or solvate thereof, wherein R² is —C(O)N(H)R¹⁰, wherein R¹⁰ is alkyl that is unsubstituted. In some embodiments is a compound of Formula (I), or a pharmaceutically acceptable salt or solvate thereof, wherein R² is —C(O)N(R⁹)R¹⁰, wherein each of R⁹ and R¹⁰ is independently alkyl substituted with —N(R¹⁸)R¹⁹ or —C(O)OR¹³. In some embodiments is a compound of Formula (I), or a pharmaceutically acceptable salt or solvate thereof, wherein R² is —C(O)N(R⁹)R¹⁰, wherein R⁹ is unsubstituted alkyl, and R¹⁰ is alkyl substituted with —N(R¹⁸)R¹⁹ or —C(O)OR¹³. In some embodiments is a compound of Formula (I), or a pharmaceutically acceptable salt or solvate thereof, wherein R² is —C(O)N(H)R¹⁰, wherein R¹⁰ is alkyl substituted with —N(R¹⁸)R¹⁹ or —C(O)OR¹³. In some embodiments is a compound of Formula (I), or a pharmaceutically acceptable salt or solvate thereof, wherein R² is —C(O)N(R⁹)R¹⁰, wherein R⁹ is alkyl, and R¹⁰ is alkyl substituted with —N(R¹⁸)R¹⁹, wherein each of R¹⁸ and R¹⁹ is alkyl. In some embodiments is a compound of Formula (I), or a pharmaceutically acceptable salt or solvate thereof, wherein R² is —C(O)N(R⁹)R¹⁰, wherein R⁹ is alkyl, and R¹⁰ is alkyl substituted with —N(R¹⁸)R¹⁹, wherein each of R¹⁸ and R¹⁹ is alkyl that is unsubstituted. In some embodiments is a compound of Formula (I), or a pharmaceutically acceptable salt or solvate thereof, wherein R² is —C(O)N(R⁹)R¹⁰, wherein R⁹ is unsubstituted alkyl, and R¹⁰ is alkyl substituted with —N(R¹⁸)R¹⁹, wherein each of R¹⁸ and R¹⁹ is alkyl that is unsubstituted. In some embodiments is a compound of Formula (I), or a pharmaceutically acceptable salt or solvate thereof, wherein R² is —C(O)N(H)R¹⁰, wherein R¹⁰ is alkyl substituted with —N(R¹⁸)R¹⁹, wherein each of R¹⁸ and R¹⁹ is alkyl that is unsubstituted. In some embodiments is a compound of Formula (I), or a pharmaceutically acceptable salt or solvate thereof, wherein R² is —C(O)N(R⁹)R¹⁰, wherein R⁹ is alkyl, and R¹⁰ is alkyl substituted with —C(O)OR¹³, wherein R¹³ is alkyl or hydrogen. In some embodiments is a compound of Formula (I), or a pharmaceutically acceptable salt or solvate thereof, wherein R² is —C(O)N(R⁹)R¹⁰, wherein R⁹ is alkyl, and R¹⁰ is alkyl substituted with —C(O)OR¹³, wherein R¹³ is alkyl that is unsubstituted, or hydrogen. In some embodiments is a compound of Formula (I), or a pharmaceutically acceptable salt or solvate thereof, wherein R² is —C(O)N(H)R¹⁰, wherein R¹⁰ is alkyl substituted with —C(O)OR¹³, wherein R¹³ is alkyl that is unsubstituted, or hydrogen. In some embodiments is a compound of Formula (I), or a pharmaceutically acceptable salt or solvate thereof, wherein R² is —C(O)N(R⁹)R¹⁰, wherein each of R⁹ and R¹⁰ is independently alkyl substituted with —C(O)OH. In some embodiments is a compound of Formula (I), or a pharmaceutically acceptable salt or solvate thereof, wherein R² is —C(O)N(R⁹)R¹⁰, wherein R⁹ is alkyl, and R¹⁰ is alkyl substituted with —C(O)OH. In some embodiments is a compound of Formula (I), or a pharmaceutically acceptable salt or solvate thereof, wherein R² is C(O)N(H)R¹⁰, wherein R¹⁰ is alkyl substituted with —C(O)OH.

In some embodiments is a compound of Formula (I), or a pharmaceutically acceptable salt or solvate thereof, wherein R² is —C(O)N(R⁹)R¹⁰. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl. In some embodiments is a compound of Formula (I), or a pharmaceutically acceptable salt or solvate thereof, wherein R² is —C(O)N(R⁹)R¹⁰, wherein R⁹ is hydrogen, unsubstituted aryl, unsubstituted heteroaryl, unsubstituted alkyl, or unsubstituted heteroalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heteroalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heterocyclylalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with cycloalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heteroalkyl that is unsubstituted. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heterocyclylalkyl that is unsubstituted. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with cycloalkyl that is unsubstituted. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heteroalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with heterocyclylalkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with cycloalkyl substituted with alkyl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with —OC(O)R¹⁵. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with —OC(O)R¹⁵, wherein R¹⁵ is hydrogen, alkyl, aryl, or heteroaryl. In some embodiments is a compound of Formula (I), or a pharmaceutically acceptable salt or solvate thereof, wherein R² is —C(O)N(R⁹)R¹⁰, wherein R⁹ is hydrogen, aryl, heteroaryl, alkyl, or heteroalkyl, each of which is substituted with —OC(O)R¹⁵, wherein R¹⁵ is hydrogen, unsubstituted alkyl, unsubstituted aryl, or unsubstituted heteroaryl.

In some embodiments is a compound of Formula (I), or a pharmaceutically acceptable salt or solvate thereof, wherein R² is —C(O)N(R⁹)R¹⁰, wherein R⁹ is hydrogen, alkyl, cycloalkyl, or heteroalkyl. In some embodiments is a compound of Formula (I), or a pharmaceutically acceptable salt or solvate thereof, wherein R² is —C(O)N(R⁹)R¹⁰, wherein R⁹ is hydrogen, unsubstituted alkyl, unsubstituted cycloalkyl, or unsubstituted heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl, each of which is substituted with —N(R¹³)C(O)R¹⁴, wherein each of R¹³ and R¹⁴ is independently hydrogen, aryl, heteroaryl, alkyl, or heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl, each of which is substituted with —C(O)N(R¹⁸)R¹⁹, wherein each of R¹⁸ and R¹⁹ is independently hydrogen, aryl, heteroaryl, alkyl, or heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl, each of which is substituted with —N(R¹³)C(O)R¹⁴, wherein each of R¹³ and R¹⁴ is independently hydrogen, unsubstituted aryl, unsubstituted heteroaryl, unsubstituted alkyl, or unsubstituted heteroalkyl. In some embodiments is a compound of Formula (I), or a pharmaceutically acceptable salt or solvate thereof, wherein R² is —C(O)N(R⁹)R¹⁰, wherein R¹⁰ is alkyl or heteroalkyl, each of which is substituted with —C(O)N(R¹⁸)R¹⁹, wherein each of R¹⁸ and R¹⁹ is independently hydrogen, unsubstituted aryl, unsubstituted heteroaryl, unsubstituted alkyl, or unsubstituted heteroalkyl.

In some embodiments is a compound of Formula (I), or a pharmaceutically acceptable salt or solvate thereof, wherein R² is —C(O)N(R⁹)R¹⁰, wherein R⁹ is hydrogen, alkyl, cycloalkyl, or heteroalkyl. In some embodiments is a compound of Formula (I), or a pharmaceutically acceptable salt or solvate thereof, wherein R² is —C(O)N(R⁹)R¹⁰, wherein R⁹ is hydrogen, unsubstituted alkyl, unsubstituted cycloalkyl, or unsubstituted heteroalkyl. In some embodiments is a compound of Formula (I), or a pharmaceutically acceptable salt or solvate thereof, wherein R² is —C(O)N(R⁹)R¹⁰, wherein R¹⁰ is cycloalkyl substituted with —N(R¹⁸)R¹⁹, wherein each of R¹⁸ and R¹⁹ is hydrogen, alkyl, heteroalkyl, or cycloalkyl. In some embodiments is a compound of Formula (I), or a pharmaceutically acceptable salt or solvate thereof, wherein R² is —C(O)N(R⁹)R¹⁰, wherein R¹⁰ is cycloalkyl substituted with —N(R¹⁸)R¹⁹, wherein each of R¹⁸ and R¹⁹ is hydrogen, unsubstituted alkyl, unsubstituted heteroalkyl, or unsubstituted cycloalkyl. In some embodiments is a compound of Formula (I), or a pharmaceutically acceptable salt or solvate thereof, wherein R² is —C(O)N(R⁹)R¹⁰, wherein R¹⁰ is cycloalkyl substituted with —N(R¹⁸)R¹⁹, wherein R¹⁸ and R¹⁹ together with the atom to which they are attached form a heterocyclylalkyl ring that is unsubstituted.

In some embodiments is a compound of Formula (I), or a pharmaceutically acceptable salt or solvate thereof, wherein R² is —C(O)N(R⁹)R¹⁰, wherein R⁹ is hydrogen, alkyl, cycloalkyl, or heteroalkyl. In some embodiments is a compound of Formula (I), or a pharmaceutically acceptable salt or solvate thereof, wherein R² is —C(O)N(R⁹)R¹⁰, wherein R⁹ is hydrogen, unsubstituted alkyl, unsubstituted cycloalkyl, or unsubstituted heteroalkyl. In some embodiments is a compound of Formula (I), or a pharmaceutically acceptable salt or solvate thereof, wherein R² is —C(O)N(R⁹)R¹⁰, wherein R¹⁰ is alkyl substituted with —OC(O)N(R¹⁸)R¹⁹, wherein R¹⁸ and R¹⁹ together with the atom to which they are attached form a heteroaryl ring or a heterocyclylalkyl ring, each of which is substituted with alkyl, heteroalkyl, or cycloalkyl. In some embodiments is a compound of Formula (I), or a pharmaceutically acceptable salt or solvate thereof, wherein R² is —C(O)N(R⁹)R¹⁰, wherein R¹⁰ is alkyl substituted with —OC(O)R¹⁵, wherein R¹⁵ is heterocyclylalkyl substituted with alkyl or arylalkyl.

In some embodiments is a compound of Formula (I), or a pharmaceutically acceptable salt or solvate thereof, wherein R² is —C(O)R⁴, wherein R⁴ is alkyl, heteroalkyl, heterocyclylalkyl, or cycloalkyl. In some embodiments is a compound of Formula (I), or a pharmaceutically acceptable salt or solvate thereof, wherein R² is —C(O)R⁴, wherein R⁴ is unsubstituted alkyl, unsubstituted heteroalkyl, unsubstituted heterocyclylalkyl, or unsubstituted cycloalkyl. In some embodiments is a compound of Formula (I), or a pharmaceutically acceptable salt or solvate thereof, wherein R² is —C(O)R⁴, wherein R⁴ is heterocyclylalkyl substituted with aryl or arylalkyl. In some embodiments is a compound of Formula (I), or a pharmaceutically acceptable salt or solvate thereof, wherein R² is —C(O)R⁴, wherein R⁴ is heterocyclylalkyl substituted with aryl, heterocyclylalkyl, or arylalkyl. In some embodiments is a compound of Formula (I), or a pharmaceutically acceptable salt or solvate thereof, wherein R² is —C(O)R⁴, wherein R⁴ is heterocyclylalkyl substituted with heterocyclylalkyl.

In some embodiments is a compound of Formula (Iu), or a pharmaceutically acceptable salt or solvate thereof, wherein the compound is:

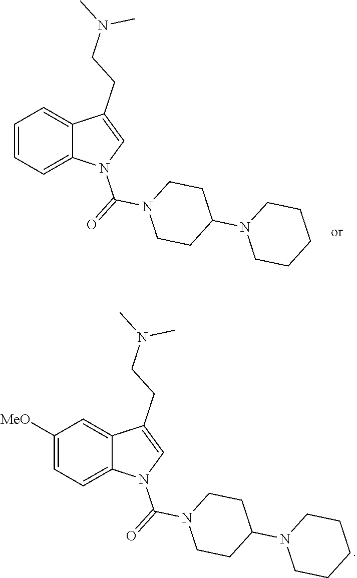 [SMILES: CN(C)CCc1cn(C(=O)N2CCC(N3CCCCC3)CC2)c2ccccc12 ; COc1ccc2c(c1)c(CCN(C)C)cn2C(=O)N1CCC(N2CCCCC2)CC1]

In some embodiments is a compound of Formula (I), or a pharmaceutically acceptable salt or solvate thereof, wherein R² is —C(O)R⁴, wherein R⁴ is alkyl substituted with —C(O)OR¹³. In some embodiments is a compound of Formula (I), or a pharmaceutically acceptable salt or solvate thereof, wherein R² is —C(O)R⁴, wherein R⁴ is alkyl substituted with —C(O)OR¹³, wherein R¹³ is hydrogen, alkyl, cycloalkyl, aryl, or heteroaryl. In some embodiments is a compound of Formula (I), or a pharmaceutically acceptable salt or solvate thereof, wherein R² is —C(O)R⁴, wherein R⁴ is alkyl substituted with —C(O)OR¹³, wherein R¹³ is hydrogen, unsubstituted alkyl, unsubstituted cycloalkyl, unsubstituted aryl, or unsubstituted heteroaryl. In some embodiments is a compound of Formula (I), or a pharmaceutically acceptable salt or solvate thereof, wherein R² is —C(O)R⁴, wherein R⁴ is alkyl substituted with —OC(O)R¹⁵, wherein R¹⁵ is alkyl, cycloalkyl, heteroaryl, or heterocyclylalkyl. In some embodiments is a compound of Formula (I), or a pharmaceutically acceptable salt or solvate thereof, wherein R² is —C(O)R⁴, wherein R⁴ is alkyl substituted with —OC(O)R¹⁵, wherein R¹⁵ is alkyl, cycloalkyl, heteroaryl, or heterocyclylalkyl. In some embodiments is a compound of Formula (I), or a pharmaceutically acceptable salt or solvate thereof, wherein R² is —C(O)R⁴, wherein R⁴ is alkyl substituted with —OC(O)R¹⁵, wherein R¹⁵ is unsubstituted alkyl, unsubstituted cycloalkyl, unsubstituted heteroaryl, or unsubstituted heterocyclylalkyl. In some embodiments is a compound of Formula (I), or a pharmaceutically acceptable salt or solvate thereof, wherein R² is —C(O)R⁴, wherein R⁴ is alkyl substituted with —OC(O)R¹⁵, wherein R¹⁵ is heterocyclylalkyl substituted with alkyl. In some embodiments is a compound of Formula (I), or a pharmaceutically acceptable salt or solvate thereof, wherein R² is —C(O)R⁴, wherein R⁴ is alkyl substituted with —N(R¹³)C(O)R¹⁴, wherein R¹³ is alkyl, cycloalkyl, or hydrogen; and R¹⁴ is alkyl, aryl, or heteroaryl. In some embodiments is a compound of Formula (I), or a pharmaceutically acceptable salt or solvate thereof, wherein R² is —C(O)R⁴, wherein R⁴ is alkyl substituted with —N(R¹³)C(O)R¹⁴, wherein R¹³ is unsubstituted alkyl, unsubstituted cycloalkyl, or hydrogen; and R¹⁴ is unsubstituted alkyl, unsubstituted aryl, or unsubstituted heteroaryl. In some embodiments is a compound of Formula (I), or a pharmaceutically acceptable salt or solvate thereof, wherein R² is —C(O)R⁴, wherein R⁴ is alkyl substituted with —NH₂. In some embodiments is a compound of Formula (I), or a pharmaceutically acceptable salt or solvate thereof, wherein R² is —C(O)R⁴, wherein R⁴ is alkyl substituted with aryl, wherein the aryl is substituted with alkyl or —OC(O)OR¹⁶. In some embodiments is a compound of Formula (I), or a pharmaceutically acceptable salt or solvate thereof, wherein R² is —C(O)R⁴, wherein R⁴ is alkyl substituted with aryl, wherein the aryl is substituted with alkyl or —OC(O)OR¹⁶, wherein R¹⁶ is alkyl, heteroalkyl, cycloalkyl, aryl, or heteroaryl. In some embodiments is a compound of Formula (I), or a pharmaceutically acceptable salt or solvate thereof, wherein R² is —C(O)R⁴, wherein R⁴ is alkyl substituted with aryl, wherein the aryl is substituted with alkyl or —OC(O)OR¹⁶, wherein R¹⁶ is unsubstituted alkyl, unsubstituted heteroalkyl, unsubstituted cycloalkyl, unsubstituted aryl, or unsubstituted heteroaryl.

In some embodiments is a compound of Formula (I), or a pharmaceutically acceptable salt or solvate thereof, wherein R² is —C(O)R⁴, wherein R⁴ is heterocyclylalkyl substituted with C(O)R¹⁴. In some embodiments is a compound of Formula (I), or a pharmaceutically acceptable salt or solvate thereof, wherein R² is —C(O)R⁴, wherein R⁴ is heterocyclylalkyl substituted with C(O)R¹⁴, wherein R¹⁴ is alkyl, heteroalkyl, cycloalkyl, or aryl. In some embodiments is a compound of Formula (I), or a pharmaceutically acceptable salt or solvate thereof, wherein R² is —C(O)R⁴, wherein R⁴ is heterocyclylalkyl substituted with C(O)R¹⁴, wherein R¹⁴ is unsubstituted alkyl, unsubstituted heteroalkyl, unsubstituted cycloalkyl, or unsubstituted aryl.

In some embodiments is a compound of Formula (I) or a pharmaceutically acceptable salt thereof, wherein R² is —P(O)OR¹¹(OR¹²) or CH(R⁵)OP(O)OR¹¹(OR¹²), wherein R¹¹ is hydrogen, and R¹² is alkyl, heteroalkyl, cycloalkyl, heterocyclylalkyl, aryl, or heteroaryl, wherein alkyl, heteroalkyl, cycloalkyl, heterocyclylalkyl, aryl, or heteroaryl is unsubstituted or substituted with one or more R^{A}. In some embodiments is a compound of Formula (I) or a pharmaceutically acceptable salt thereof, wherein R² is —P(O)OR¹¹(OR¹²) or CH(R⁵)OP(O)OR¹¹(OR¹²), wherein R¹¹ is hydrogen, and R¹² is alkyl that is unsubstituted or substituted with one or more R^{A}.

In some embodiments is a compound of Formula (I) or (Iu), or a pharmaceutically acceptable salt thereof, wherein R²⁰ is hydrogen, and R²¹ is alkyl, alkyl, heteroalkyl, cycloalkyl, heterocyclylalkyl, aryl, heteroaryl, or hydrogen, wherein alkyl, heteroalkyl, cycloalkyl, heterocyclylalkyl, aryl, or heteroaryl is unsubstituted or substituted with one or more R^{B}, or R²⁰ and R²¹ together with the atoms to which they are attached form a heterocyclylalkyl ring that is unsubstituted or substituted with one or more R^{B}. In some embodiments is a compound of Formula (I) or (Iu) or a pharmaceutically acceptable salt thereof, wherein R²⁰ is hydrogen, and R²¹ is alkyl that is unsubstituted or substituted with one or more R^{B}.

In some embodiments is a compound of Formula (I), or a pharmaceutically acceptable salt thereof, wherein R² is —CH(R⁵)OC(O)OR⁶. In some embodiments is a compound of Formula (I), or a pharmaceutically acceptable salt thereof, wherein R² is —CH(R⁵)OC(O)OR⁶, wherein each of R⁵ and R⁶ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently unsubstituted or substituted with one or more R^{A}. In some embodiments is a compound of Formula (I), or a pharmaceutically acceptable salt thereof, wherein R² is —CH(R⁵)OC(O)OR⁶, wherein each of R⁵ and R⁶ is independently hydrogen or alkyl that is unsubstituted or substituted with one or more R^{A}. In some embodiments is a compound of Formula (I), or a pharmaceutically acceptable salt thereof, wherein R² is —CH(R⁵)OC(O)OR⁶, wherein R⁵ is hydrogen and R⁶ is hydrogen or alkyl that is unsubstituted or substituted with one or more R^{A}. In some embodiments is a compound of Formula (I), or a pharmaceutically acceptable salt thereof, wherein R² is —CH(R⁵)OC(O)OR⁶, wherein R⁵ is hydrogen and R⁶ is methyl, ethyl, n-propyl, isopropyl, isobutyl, tert-butyl, or n-butyl. In some embodiments is a compound of Formula (I), or a pharmaceutically acceptable salt thereof, wherein R² is —CH(R⁵)OC(O)OR⁶, wherein R⁵ is hydrogen and R⁶ is ethyl.

In some embodiments is a compound of Formula (I), or a pharmaceutically acceptable salt or solvate thereof, wherein R² is —CH(R⁵)OP(O)OR¹¹(OR¹²). In some embodiments is a compound of Formula (I), or a pharmaceutically acceptable salt or solvate thereof, wherein R² is —CH(R⁵)OP(O)OR¹¹(OR¹²), wherein R⁵ is hydrogen, alkyl, cycloalkyl, or heteroalkyl. In some embodiments is a compound of Formula (I), or a pharmaceutically acceptable salt or solvate thereof, wherein R² is —CH(R⁵)OP(O)OR¹¹(OR¹²), wherein R⁵ is hydrogen, unsubstituted alkyl, unsubstituted cycloalkyl, unsubstituted heteroalkyl, or alkyl substituted with heteroaryl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is independently selected from alkyl, cycloalkyl, aryl, heteroaryl, or alkyl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is independently selected from unsubstituted alkyl, unsubstituted cycloalkyl, unsubstituted aryl, unsubstituted heteroaryl, unsubstituted alkyl, or alkyl substituted with aryl or heteroaryl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is alkyl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is unsubstituted alkyl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is alkyl substituted with —OC(O)R¹⁵. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is alkyl substituted with —OC(O)R¹⁵, wherein each R¹⁵ is alkyl, cycloalkyl, heterocyclylalkyl, aryl, or heteroaryl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is alkyl substituted with —OC(O)R¹⁵, wherein each R¹⁵ is unsubstituted alkyl, unsubstituted cycloalkyl, unsubstituted heterocyclylalkyl, unsubstituted aryl, or unsubstituted heteroaryl. In some embodiments is a compound of Formula (I), or a pharmaceutically acceptable salt or solvate thereof, wherein R² is —CH(R⁵)OP(O)OR¹¹(OR¹²), wherein each of R¹¹ and R¹² is alkyl substituted with —OC(O)R¹⁵, wherein each R¹⁵ is heterocyclylalkyl substituted with alkyl or arylalkyl.

In some embodiments is a compound of Formula (I), or a pharmaceutically acceptable salt or solvate thereof, wherein R² is —CH(R⁵)OP(O)OR⁸[N(R⁹)R¹⁰]. In some embodiments is a compound of Formula (I), or a pharmaceutically acceptable salt or solvate thereof, wherein R² is —CH(R⁵)OP(O)OR⁸[N(R⁹)R¹⁰], wherein R⁵ is hydrogen, alkyl, cycloalkyl, heteroalkyl, or alkyl substituted with heteroaryl. In some embodiments is a compound of Formula (I), or a pharmaceutically acceptable salt or solvate thereof, wherein R² is —CH(R⁵)OP(O)OR⁸[N(R⁹)R¹⁰], wherein R⁴ is hydrogen, unsubstituted alkyl, unsubstituted cycloalkyl, unsubstituted heteroalkyl, or alkyl substituted with heteroaryl. In some embodiments is a compound of Formula (I), or a pharmaceutically acceptable salt or solvate thereof, wherein R² is —CH(R⁵)OP(O)OR⁸[N(R⁹)R¹⁰], wherein R⁸ is alkyl, cycloalkyl, aryl, heteroaryl, alkyl, or alkyl substituted with aryl or heteroaryl; R⁹ is hydrogen; and R¹² is alkyl substituted with —C(O)OR¹³. In some embodiments is a compound of Formula (I), or a pharmaceutically acceptable salt or solvate thereof, wherein R² is —CH(R⁵)OP(O)OR⁸[N(R⁹)R¹⁰], wherein R⁸ is unsubstituted alkyl, unsubstituted cycloalkyl, unsubstituted aryl, unsubstituted heteroaryl, unsubstituted alkyl, or alkyl substituted with aryl or heteroaryl; R⁹ is hydrogen; and R¹² is alkyl substituted with —C(O)OR¹³. In some embodiments is a compound of Formula (I), or a pharmaceutically acceptable salt or solvate thereof, wherein R¹³ is alkyl. In some embodiments is a compound of Formula (I), or a pharmaceutically acceptable salt or solvate thereof, wherein R² is —CH(R⁵)OP(O)OR⁸[N(R⁹)R¹⁰], wherein R¹² is alkyl substituted with —C(O)OR¹³, wherein R¹³ is unsubstituted alkyl.

In some embodiments is a compound of Formula (I), or a pharmaceutically acceptable salt or solvate thereof, wherein R² is —P(O)OR¹¹(OR¹²). In some embodiments is a compound of Formula (I), or a pharmaceutically acceptable salt or solvate thereof, wherein R² is —P(O)OR¹¹(OR¹²), wherein each of R¹¹ and R¹² is alkyl. In some embodiments is a compound of Formula (I), or a pharmaceutically acceptable salt or solvate thereof, wherein R² is —P(O)OR¹¹(OR¹²), wherein each of R¹¹ and R¹² is unsubstituted alkyl. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —C(O)OR¹³. In some embodiments is a compound of Formula (I), or a pharmaceutically acceptable salt or solvate thereof, wherein R² is —P(O)OR¹¹(OR¹²), wherein R¹³ is alkyl, cycloalkyl, aryl, or heteroaryl. In some embodiments is a compound of Formula (I), or a pharmaceutically acceptable salt or solvate thereof, wherein R² is —P(O)OR¹¹(OR¹²), wherein R¹³ is unsubstituted alkyl, unsubstituted cycloalkyl, unsubstituted aryl, or unsubstituted heteroaryl. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —OC(O)R¹⁵. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —OC(O)R¹⁵, wherein R¹⁵ is alkyl, cycloalkyl, heteroaryl, or heterocyclylalkyl. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —OC(O)R¹⁵, wherein R¹⁵ is unsubstituted alkyl, unsubstituted cycloalkyl, unsubstituted heteroaryl, or unsubstituted heterocyclylalkyl. In some embodiments is a compound of Formula (I), or a pharmaceutically acceptable salt or solvate thereof, wherein R² is —P(O)OR¹¹(OR²), wherein each of R¹¹ and R¹² is alkyl substituted with —OC(O)R⁵, wherein R¹⁵ is heterocyclylalkyl substituted with alkyl or arylalkyl. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —OC(O)OR¹⁶, wherein R¹⁶ is alkyl, cycloalkyl, heteroaryl, or heterocyclylalkyl. In some embodiments is a compound of Formula (I), or a pharmaceutically acceptable salt or solvate thereof, wherein R² is —P(O)OR¹¹(OR¹²), wherein each of R¹¹ and R¹² is alkyl substituted with —OC(O)OR¹⁶, wherein R¹⁶ is unsubstituted alkyl, unsubstituted cycloalkyl, unsubstituted heteroaryl, or unsubstituted heterocyclylalkyl.

In some embodiments is a compound of Formula (I), or a pharmaceutically acceptable salt or solvate thereof, wherein R² is —P(O)OR¹¹(OR²), wherein R¹¹ and R¹² together with the atom to which they are attached form a heterocyclylalkyl ring. In some embodiments is a compound of Formula (I), or a pharmaceutically acceptable salt or solvate thereof, wherein R² is —P(O)OR¹¹(OR²), wherein R¹¹ and R¹² together with the atom to which they are attached form a heterocyclylalkyl ring that is unsubstituted. In some embodiments is a compound of Formula (I), or a pharmaceutically acceptable salt or solvate thereof, wherein R² is —P(O)OR¹¹(OR²), wherein R¹¹ and R¹² together with the atom to which they are attached form a heterocyclylalkyl ring that is substituted with aryl. In some embodiments is a compound of Formula (I), or a pharmaceutically acceptable salt or solvate thereof, wherein R² is —P(O)OR¹¹(OR²), wherein R¹¹ and R¹² together with the atom to which they are attached form a heterocyclylalkyl ring that is substituted with unsubstituted aryl. In some embodiments is a compound of Formula (I), or a pharmaceutically acceptable salt or solvate thereof, wherein R² is —P(O)OR¹¹(OR¹²), wherein R¹¹ and R¹² together with the atom to which they are attached form a heterocyclylalkyl ring that is substituted with aryl, wherein the aryl is substituted with halogen.

In some embodiments is a compound of Formula (I), or a pharmaceutically acceptable salt or solvate thereof, wherein R² is —P(O)OR⁸[N(R⁹)R¹⁰]. In some embodiments is a compound of Formula (I), or a pharmaceutically acceptable salt or solvate thereof, wherein R² is —P(O)OR⁸[N(R⁹)R¹⁰], wherein R⁸ is alkyl, aryl, or heteroaryl. In some embodiments is a compound of Formula (I), or a pharmaceutically acceptable salt or solvate thereof, wherein R² is —P(O)OR⁸[N(R⁹)R¹⁰], wherein R⁸ is unsubstituted alkyl, unsubstituted aryl, or unsubstituted heteroaryl. In some embodiments is a compound of Formula (I), or a pharmaceutically acceptable salt or solvate thereof, wherein R² is —P(O)OR⁸[N(R⁹)R¹⁰], wherein each of R⁹ and R¹⁰ are independently selected from hydrogen or alkyl. In some embodiments is a compound of Formula (I), or a pharmaceutically acceptable salt or solvate thereof, wherein R² is —P(O)OR⁸[N(R⁹)R¹⁰], wherein R⁸ is unsubstituted alkyl, unsubstituted aryl, or unsubstituted heteroaryl, R⁹ is hydrogen, and R¹⁰ is alkyl. In some embodiments is a compound of Formula (I), or a pharmaceutically acceptable salt or solvate thereof, wherein R² is —P(O)OR⁸[N(R⁹)R¹⁰], wherein R⁸ is unsubstituted alkyl, unsubstituted aryl, or unsubstituted heteroaryl, R⁹ is hydrogen, and R¹⁰ is alkyl substituted with —C(O)R¹⁴. In some embodiments is a compound of Formula (I), or a pharmaceutically acceptable salt or solvate thereof, wherein R² is —P(O)OR⁸[N(R⁹)R¹⁰], wherein R¹⁰ is alkyl substituted with —C(O)R¹⁴, wherein R¹⁴ is hydrogen or alkyl. In some embodiments is a compound of Formula (I), or a pharmaceutically acceptable salt or solvate thereof, wherein R¹⁴ is unsubstituted alkyl.

In some embodiments is a compound of Formula (I), or a pharmaceutically acceptable salt or solvate thereof, wherein R² is —S(O)₂OR⁷. In some embodiments is a compound of Formula (I), or a pharmaceutically acceptable salt or solvate thereof, wherein R² is —S(O)₂OR⁷, wherein R⁷ is alkyl. In some embodiments is a compound of Formula (I), or a pharmaceutically acceptable salt or solvate thereof, wherein R² is —S(O)₂OR⁷, wherein R⁷ is alkyl substituted with —C(O)R¹⁴. In some embodiments is a compound of Formula (I), or a pharmaceutically acceptable salt or solvate thereof, wherein R⁷ is alkyl substituted with —C(O)R¹⁴, wherein R¹⁴ is alkyl, heteroalkyl, cycloalkyl, heterocyclylalkyl, aryl, or heteroaryl. In some embodiments is a compound of Formula (I), or a pharmaceutically acceptable salt or solvate thereof, wherein R² is —S(O)₂OR⁷, wherein R⁷ is alkyl substituted with —C(O)R¹⁴. In some embodiments is a compound of Formula (I), or a pharmaceutically acceptable salt or solvate thereof, wherein R⁷ is alkyl substituted with —C(O)R¹⁴, wherein R¹⁴ is heterocyclylalkyl. In some embodiments is a compound of Formula (I), or a pharmaceutically acceptable salt or solvate thereof, wherein R² is —S(O)₂OR⁷, wherein R⁷ is alkyl substituted with —C(O)R¹⁴. In some embodiments is a compound of Formula (I), or a pharmaceutically acceptable salt or solvate thereof, wherein R⁷ is alkyl substituted with —C(O)R¹⁴, wherein R¹⁴ is heterocyclylalkyl substituted with alkyl, —C(O)CH₃, or C(O)Ph.

In some embodiments is a compound of Formula ((I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, wherein R¹ is hydrogen. In some embodiments is a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, wherein R¹ is methoxy.

In some embodiments is a compound of Formula (I), or a pharmaceutically acceptable salt or solvate thereof, wherein R² is —C(O)OR³, wherein R³ is alkyl substituted with —OP(O)OR²⁰(OR²¹). In some embodiments is a compound of Formula (I), or a pharmaceutically acceptable salt or solvate thereof, wherein R² is —C(O)OR³, wherein R³ is alkyl substituted with —OP(O)OR²⁰(OR²¹), wherein each of R²⁰ and R²¹ is independently alkyl, cycloalkyl, aryl, heterocyclylalkyl, or heteroaryl. In some embodiments is a compound of Formula (I), or a pharmaceutically acceptable salt or solvate thereof, wherein R² is —C(O)OR³, wherein R³ is alkyl substituted with —OP(O)OR²⁰(OR²¹), wherein each of R²⁰ and R²¹ is independently alkyl. In some embodiments is a compound of Formula (I), or a pharmaceutically acceptable salt or solvate thereof, wherein R² is —C(O)OR³, wherein R³ is alkyl substituted with —OP(O)OR²⁰(OR²¹), wherein each of R²⁰ and R²¹ is independently unsubstituted alkyl, unsubstituted cycloalkyl, unsubstituted aryl, unsubstituted heterocyclylalkyl, or unsubstituted heteroaryl. In some embodiments is a compound of Formula (I), or a pharmaceutically acceptable salt or solvate thereof, wherein R² is —C(O)OR³, wherein R³ is alkyl substituted with —OP(O)OR²⁰(OR²¹), wherein each of R²⁰ and R²¹ is independently unsubstituted alkyl.

In another aspect, the present disclosure provides a pharmaceutically acceptable composition comprising a compound according to any of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof, and a pharmaceutically acceptable excipient, carrier, adjuvant, or vehicle.

Pharmaceutical compositions of the present disclosure can comprise racemic, scalemic, or diastereomerically enriched mixtures of any compound described herein comprising a stereogenic center.

Selected compounds of the disclosure with corresponding simplified molecular-input line-entry system (SMILES) strings are provided in TABLE 1.

TABLE 1

| | Structure |
|---|---|
| Cpd | SMILES* |
| 1 | 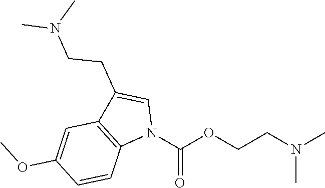 |
| | CN(CCC1═CN(C2═C1C═C(OC)C═C2)C(OCCN(C)C)═O)C |
| 2 | 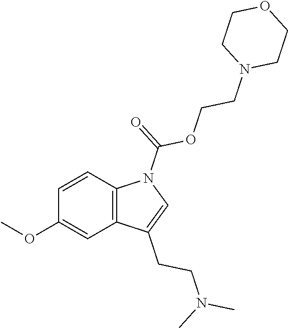 [SMILES: COc1ccc2c(c1)c(CCN(C)C)cn2C(=O)OCCN1CCOCC1] |
| | CN(CCC1═CN(C2═C1C═C(OC)C═C2)C(OCCN3CCOCC3)═O)C |
| 3 | 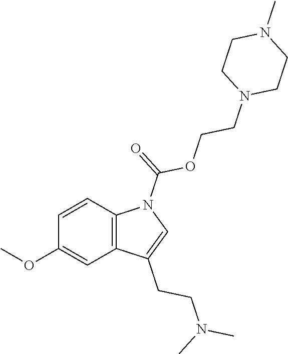 [SMILES: COc1ccc2c(c1)c(CCN(C)C)cn2C(=O)OCCN1CCN(C)CC1] |
| | CN(CCC1═CN(C2═C1C═C(OC)C═C2)C(OCCN3CCN(CC3)C)═O)C |
| 4 | 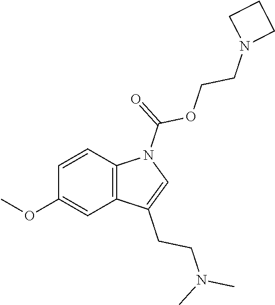 [SMILES: COc1ccc2c(c1)c(CCN(C)C)cn2C(=O)OCCN1CCC1] |
| | CN(CCC1═CN(C2═C1C═C(OC)C═C2)C(OCCN3CCC3)═O)C |
| 5 | 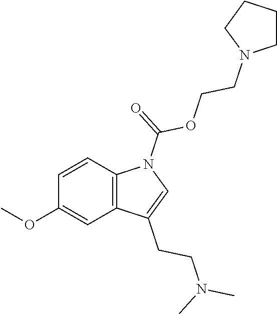 [SMILES: COc1ccc2c(c1)c(CCN(C)C)cn2C(=O)OCCN1CCCC1] |
| | CN(CCC1═CN(C2═C1C═C(OC)C═C2)C(OCCN3CCCC3)═O)C |
| 6 | 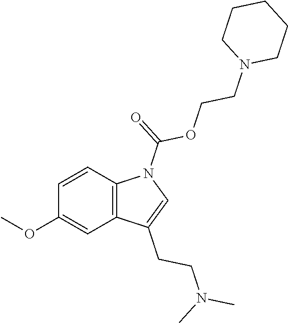 [SMILES: COc1ccc2c(c1)c(CCN(C)C)cn2C(=O)OCCN1CCCCC1] |
| | CN(CCC1═CN(C2═C1C═C(OC)C═C2)C(OCCN3CCCCC3)═O)C |
| 7 | 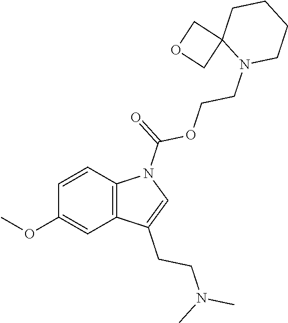 [SMILES: COc1ccc2c(c1)c(CCN(C)C)cn2C(=O)OCCN1CCCCC12COC2] |
| | CN(CCC1═CN(C2═C1C═C(OC)C═C2)C(OCCN3CCCCC34COC4)═O)C |
| 8 | 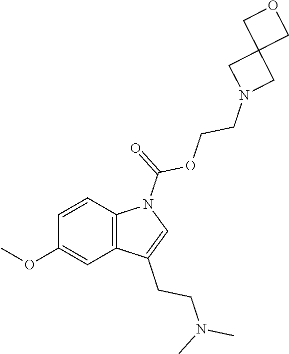 [SMILES: COc1ccc2c(c1)c(CCN(C)C)cn2C(=O)OCCN1CC2(COC2)C1] |
| | CN(CCC1═CN(C2═C1C═C(OC)C═C2)C(OCCN3CC4(C3)COC4)═O)C |
| 9 | 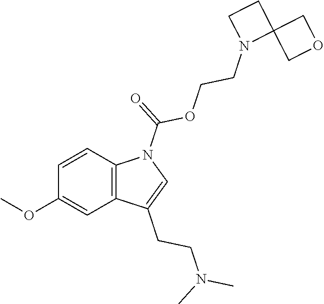 [SMILES: COc1ccc2c(c1)c(CCN(C)C)cn2C(=O)OCCN1CCC12COC2] |
| | CN(CCC1═CN(C2═C1C═C(OC)C═C2)C(OCCN3CCC34COC4)═O)C |
| 10 | 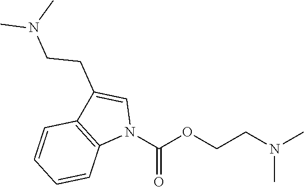 |
| | CN(CCC1═CN(C2═C1C═CC═C2)C(OCCN(C)C)═O)C |
| 11 | 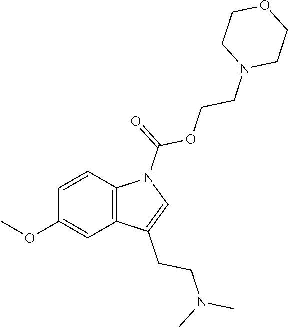 [SMILES: COc1ccc2c(c1)c(CCN(C)C)cn2C(=O)OCCN1CCOCC1] |
| | CN(CCC1═CN(C2═C1C═C(OC)C═C2)C(OCCN3CCOCC3)═O)C |
| 12 | 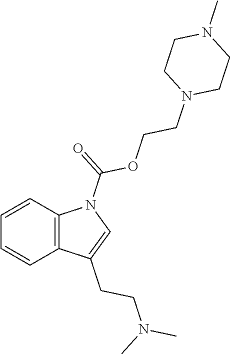 [SMILES: CN(C)CCc1cn(C(=O)OCCN2CCN(C)CC2)c2ccccc12] |
| | CN(CCC1═CN(C2═C1C═CC═C2)C(OCCN3CCN(CC3)C)═O)C |
| 13 | 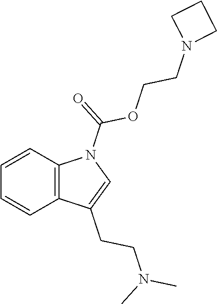 [SMILES: CN(C)CCc1cn(C(=O)OCCN2CCC2)c2ccccc12] |
| | CN(CCC1═CN(C2═C1C═CC═C2)C(OCCN3CCC3)═O)C |
| 14 | 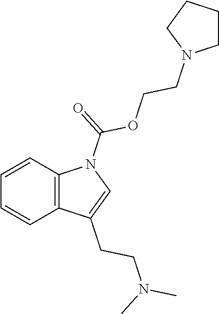 [SMILES: CN(C)CCc1cn(C(=O)OCCN2CCCC2)c2ccccc12] |
| | CN(CCC1═CN(C2═C1C═CC═C2)C(OCCN3CCCC3)═O)C |
| 15 | 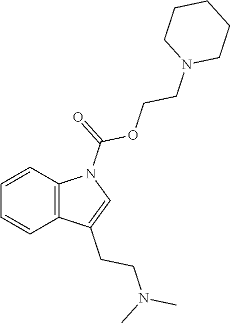 [SMILES: CN(C)CCc1cn(C(=O)OCCN2CCCCC2)c2ccccc12] |
| | CN(CCC1═CN(C2═C1C═CC═C2)C(OCCN3CCCCC3)═O)C |
| 16 | 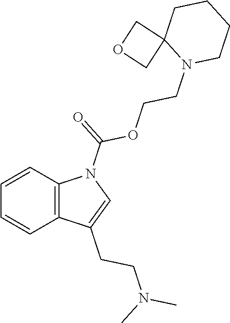 [SMILES: CN(C)CCc1cn(C(=O)OCCN2CCCCC23COC3)c2ccccc12] |
| | CN(CCC1═CN(C2═C1C═CC═C2)C(OCCN3CCCCC34COC4)═O)C |
| 17 | 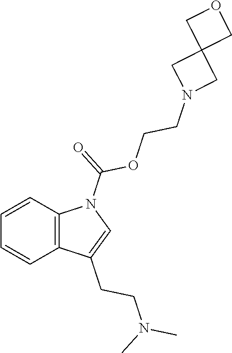 [SMILES: CN(C)CCc1cn(C(=O)OCCN2CC3(COC3)C2)c2ccccc12] |
| | CN(CCC1═CN(C2═C1C═CC═C2)C(OCCN3CCC4(C3)COC4)═O)C |
| 18 | 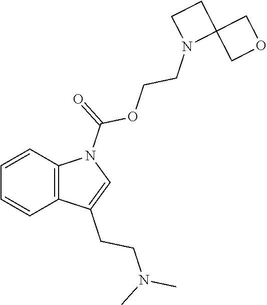 [SMILES: CN(C)CCc1cn(C(=O)OCCN2CCC23COC3)c2ccccc12] |
| | CN(CCC1═CN(C2═C1C═CC═C2)C(OCCN3CCC34COC4)═O)C |
| 19 | 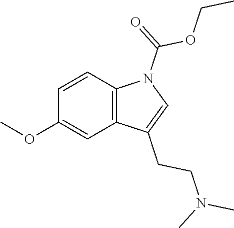 [SMILES: CCOC(=O)n1cc(CCN(C)C)c2cc(OC)ccc21] |
| | CN(CCC1═CN(C2═C1C═C(OC)C═C2)C(OCC)═O)C |
| 20 | 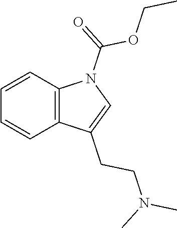 [SMILES: CCOC(=O)n1cc(CCN(C)C)c2ccccc21] |
| | CN(CCC1═CN(C2═C1C═CC═C2)C(OCC)═O)C |
| 21 | 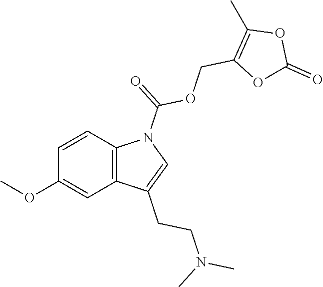 [SMILES: COc1ccc2c(c1)c(CCN(C)C)cn2C(=O)OCc1oc(=O)oc1C] |
| | CN(CCC1═CN(C2═C1C═C(OC)C═C2)C(OCC(OC3═O)═C(O3)C)═O)C |
| 22 | 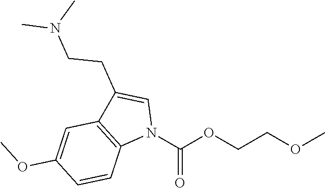 |
| | CN(CCC1═CN(C2═C1C═C(OC)C═C2)C(OCCOC)═O)C |
| 23 | 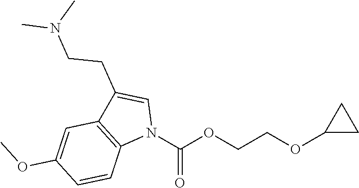 [SMILES: COc1ccc2c(c1)c(CCN(C)C)cn2C(=O)OCCOC1CC1] |
| | CN(CCC1═CN(C2═C1C═C(OC)C═C2)C(OCCOC3CC3)═O)C |
| 24 | 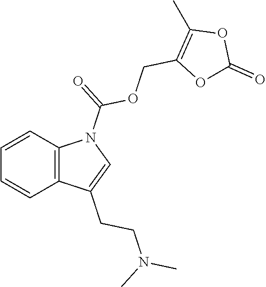 [SMILES: Cc1oc(=O)oc1COC(=O)n1cc(CCN(C)C)c2ccccc21] |
| | CN(CCC1═CN(C2═C1C═CC═C2)C(OCC(OC3═O)═C(O3)C)═O)C |
| 25 | 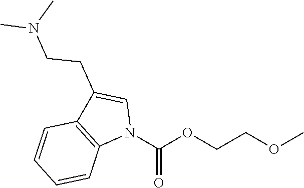 |
| | CN(CCC1═CN(C2═C1C═CC═C2)C(OCCOC)═O)C |
| 26 | 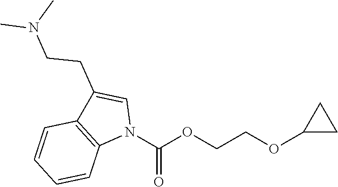 [SMILES: CN(C)CCc1cn(C(=O)OCCOC2CC2)c2ccccc12] |
| | CN(CCC1═CN(C2═C1C═CC═C2)C(OCCOC3CC3)═O)C |
| 27 | 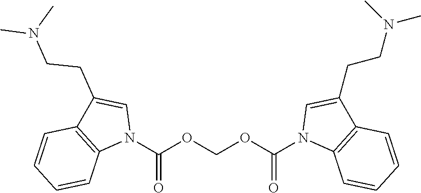 |
| | CN(CCC1═CN(C2═C1C═CC═C2)C(OCOC(N3C4═C(C(CCN(C)C)═C3)C═CC═C4)═O)═O)C |
| 28 | 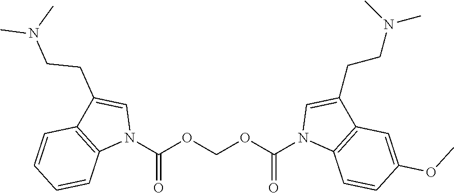 |
| | CN(CCC1═CN(C2═C1C═C(OC)C═C2)C(OCOC(N3C4═C(C(CCN(C)C)═C3)C═CC═C4)═O)═O)C |
| 29 | 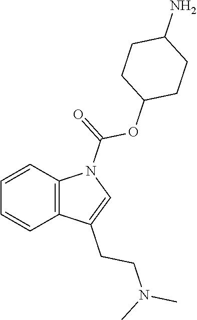 [SMILES: CN(C)CCc1cn(C(=O)OC2CCC(N)CC2)c2ccccc12] |
| | CN(CCC1═CN(C2═C1C═CC═C2)C(OC3CCC(CC3)N)═O)C |
| 30 | 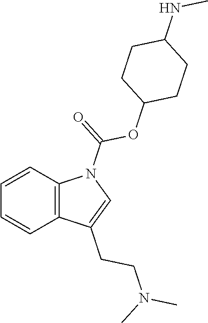 [SMILES: CNC1CCC(OC(=O)n2cc(CCN(C)C)c3ccccc32)CC1] |
| | CN(CCC1═CN(C2═C1C═CC═C2)C(OC3CCC(CC3)NC)═O)C |
| 31 | 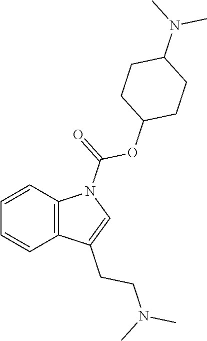 [SMILES: CN(C)CCc1cn(C(=O)OC2CCC(N(C)C)CC2)c2ccccc12] |
| | CN(CCC1═CN(C2═C1C═CC═C2)C(OC3CCC(CC3)N(C)C)═O)C |
| 32 | 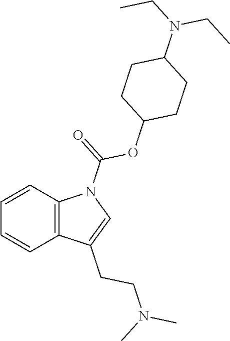 [SMILES: CCN(CC)C1CCC(OC(=O)n2cc(CCN(C)C)c3ccccc32)CC1] |
| | CN(CCC1═CN(C2═C1C═CC═C2)C(OC3CCC(CC3)N(CC)CC)═O)C |
| 33 | 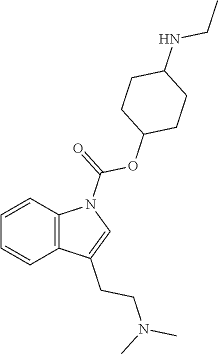 [SMILES: CCNC1CCC(OC(=O)n2cc(CCN(C)C)c3ccccc32)CC1] |
| | CN(CCC1═CN(C2═C1C═CC═C2)C(OC3CCC(CC3)NCC)═O)C |
| 34 | 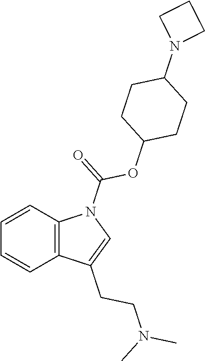 [SMILES: CN(C)CCc1cn(C(=O)OC2CCC(N3CCC3)CC2)c2ccccc12] |
| | CN(CCC1═CN(C2═C1C═CC═C2)C(OC3CCC(CC3)N4CCC4)═O)C |
| 35 | 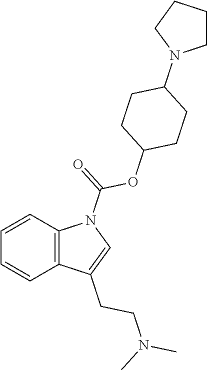 [SMILES: CN(C)CCc1cn(C(=O)OC2CCC(N3CCCC3)CC2)c2ccccc12] |
| | CN(CCC1═CN(C2═C1C═CC═C2)C(OC3CCC(CC3)N4CCCC4)═O)C |
| 36 | 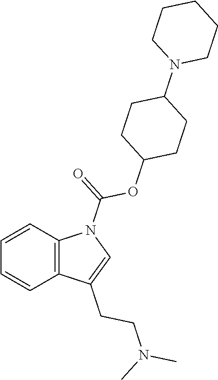 [SMILES: CN(C)CCc1cn(C(=O)OC2CCC(N3CCCCC3)CC2)c2ccccc12] |
| | CN(CCC1═CN(C2═C1C═CC═C2)C(OC3CCC(CC3)N4CCCCC4)═O)C |
| 37 | 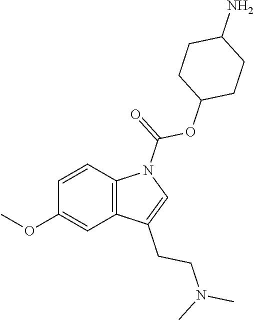 [SMILES: COc1ccc2c(c1)c(CCN(C)C)cn2C(=O)OC1CCC(N)CC1] |
| | CN(CCC1═CN(C2═C1C═C(OC)C═C2)C(OC3CCC(CC3)N)═O)C |
| 38 | 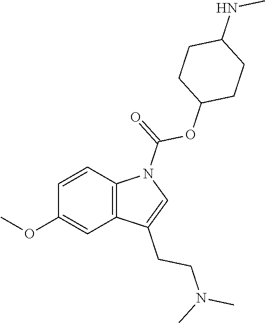 |
| | CN(CCC1═CN(C2═C1C═C(OC)C═C2)C(OC3CCC(CC3)NC)═O)C |
| 39 | 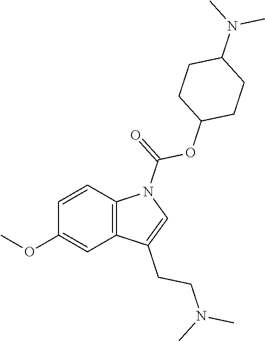 |
| | CN(CCC1═CN(C2═C1C═C(OC)C═C2)C(OC3CCC(CC3)N(C)C)═O)C |
| 40 | 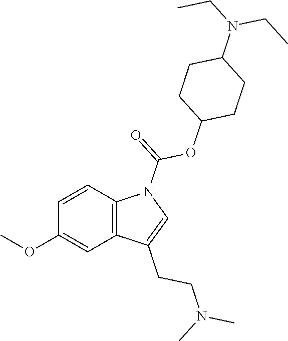 [SMILES: CCN(CC)C1CCC(OC(=O)n2cc(CCN(C)C)c3cc(OC)ccc32)CC1] |
| | CN(CCC1═CN(C2═C1C═C(OC)C═C2)C(OC3CCC(CC3)N(CC)CC)═O)C |
| 41 | 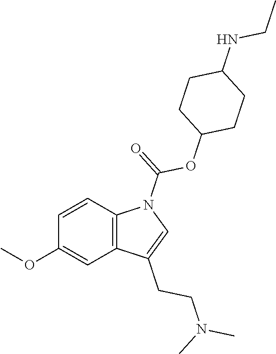 [SMILES: CCNC1CCC(OC(=O)n2cc(CCN(C)C)c3cc(OC)ccc32)CC1] |
| | CN(CCC1═CN(C2═C1C═C(OC)C═C2)C(OC3CCC(CC3)NCC)═O)C |
| 42 | 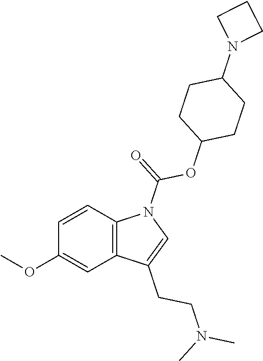 [SMILES: COc1ccc2c(c1)c(CCN(C)C)cn2C(=O)OC1CCC(N2CCC2)CC1] |
| | CN(CCC1═CN(C2═C1C═C(OC)C═C2)C(OC3CCC(CC3)N4CCC4)═O)C |
| 43 | 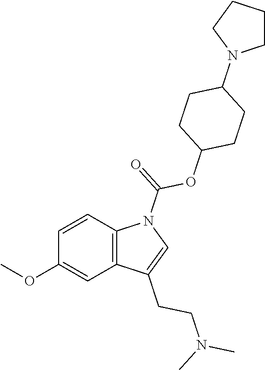 [SMILES: COc1ccc2c(c1)c(CCN(C)C)cn2C(=O)OC1CCC(N2CCCC2)CC1] |
| | CN(CCC1═CN(C2═C1C═C(OC)C═C2)C(OC3CCC(CC3)N4CCCC4)═O)C |
| 44 | 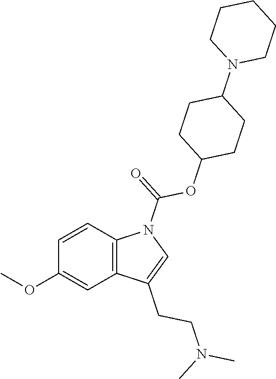 [SMILES: COc1ccc2c(c1)c(CCN(C)C)cn2C(=O)OC1CCC(N2CCCCC2)CC1] |
| | CN(CCC1═CN(C2═C1C═C(OC)C═C2)C(OC3CCC(CC3)N4CCCCC4)═O)C |
| 45 | 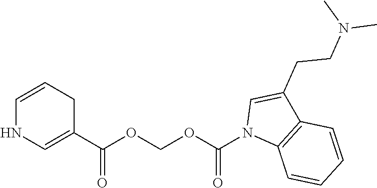 [SMILES: CN(C)CCc1cn(C(=O)OCOC(=O)C2=CNC=CC2)c2ccccc12] |
| | CN(CCC1═CN(C2═C1C═CC═C2)C(OCOC(C3═CNC═CC3)═O)═O)C |
| 46 | 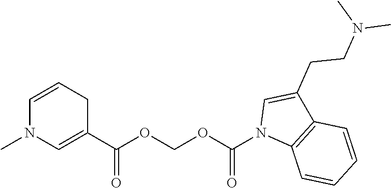 |
| | CN(CCC1═CN(C2═C1C═CC═C2)C(OCOC(C3═CN(C)C═CC3)═O)═O)C |
| 47 | 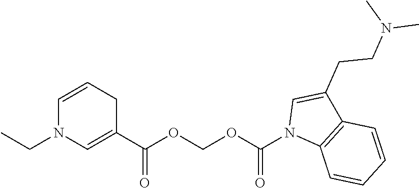 [SMILES: CCN1C=CCC(C(=O)OCOC(=O)n2cc(CCN(C)C)c3ccccc32)=C1] |
| | CN(CCC1═CN(C2═C1C═CC═C2)C(OCOC(C3═CN(C═CC3)CC)═O)═O)C |
| 48 | 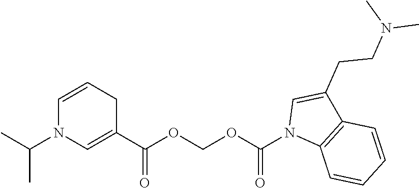 [SMILES: CC(C)N1C=CCC(C(=O)OCOC(=O)n2cc(CCN(C)C)c3ccccc32)=C1] |
| | CN(CCC1═CN(C2═C1C═CC═C2)C(OCOC(C3═CN(═CC3)C(C)C)═O)═O)C |
| 49 | 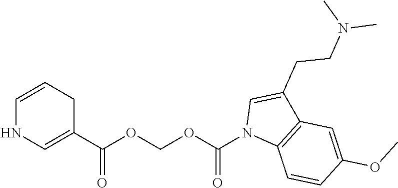 [SMILES: COc1ccc2c(c1)c(CCN(C)C)cn2C(=O)OCOC(=O)C1=CNC=CC1] |
| | CN(CCC1═CN(C2═C1C═C(OC)C═C2)C(OCOC(C3═CNC═CC3)═O)═O)C |
| 50 | 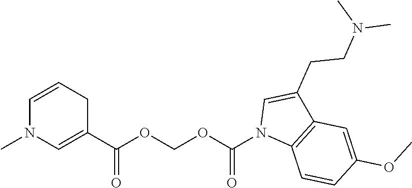 |
| | CN(CCC1═CN(C2═C1C═C(OC)C═C2)C(OCOC(C3═CN(C)C═CC3)═O)═O)C |
| 51 | 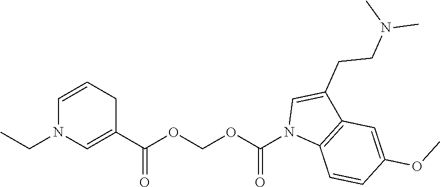 [SMILES: CCN1C=CCC(C(=O)OCOC(=O)n2cc(CCN(C)C)c3cc(OC)ccc32)=C1] |
| | CN(CCC1═CN(C2═C1C═C(OC)C═C2)C(OCOC(C3═CN(C═CC3)CC)═O)═O)C |
| 52 | 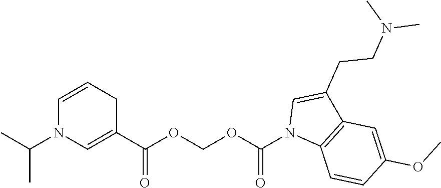 [SMILES: COc1ccc2c(c1)c(CCN(C)C)cn2C(=O)OCOC(=O)C1=CN(C(C)C)C=CC1] |
| | CN(CCC1═CN(C2═C1C═C(OC)C═C2)C(OCOC(C3═CN(C═CC3)C(C)C)═O)═O)C |
| 53 | 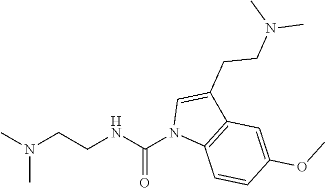 |
| | CN(CCC1═CN(C2═C1C═C(OC)C═C2)C(NCCN(C)C)═O)C |
| 54 | 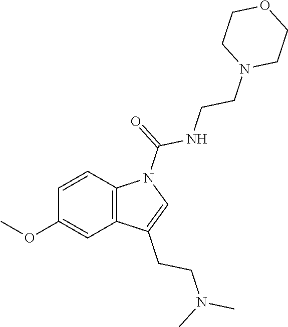 [SMILES: COc1ccc2c(c1)c(CCN(C)C)cn2C(=O)NCCN1CCOCC1] |
| | CN(CCC1═CN(C2═C1C═C(OC)C═C2)C(NCCN3CCOCC3)═O)C |
| 55 | 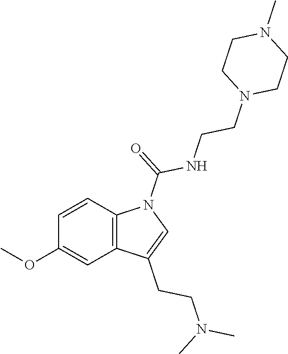 [SMILES: COc1ccc2c(c1)c(CCN(C)C)cn2C(=O)NCCN1CCN(C)CC1] |
| | CN(CCC1═CN(C2═C1C═C(OC)C═C2)C(NCCN3CCN(CC3)C)═O)C |
| 56 | 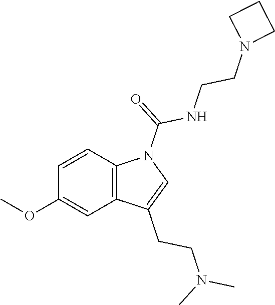 [SMILES: COc1ccc2c(c1)c(CCN(C)C)cn2C(=O)NCCN1CCC1] |
| | CN(CCC1═CN(C2═C1C═C(OC)C═C2)C(NCCN3CCC3)═O)C |
| 57 | 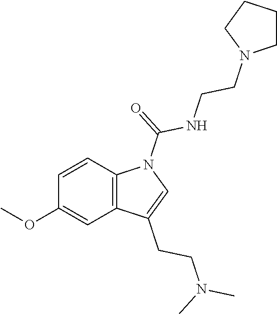 [SMILES: COc1ccc2c(c1)c(CCN(C)C)cn2C(=O)NCCN1CCCC1] |
| | CN(CCC1═CN(C2═C1C═C(OC)C═C2)C(NCCN3CCCC3)═O)C |
| 58 | 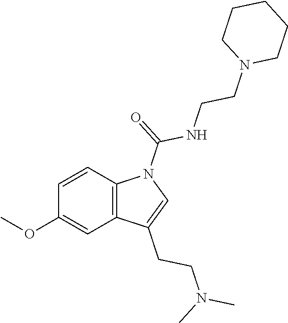 [SMILES: COc1ccc2c(c1)c(CCN(C)C)cn2C(=O)NCCN1CCCCC1] |
| | CN(CCC1═CN(C2═C1C═C(OC)C═C2)C(NCCN3CCCCC3)═O)C |
| 59 | 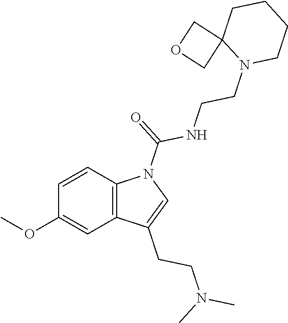 [SMILES: COc1ccc2c(c1)c(CCN(C)C)cn2C(=O)NCCN1CCCCC12COC2] |
| | CN(CCC1═CN(C2═C1C═C(OC)C═C2)C(NCCN3CCCCC34COC4)═O)C |
| 60 | 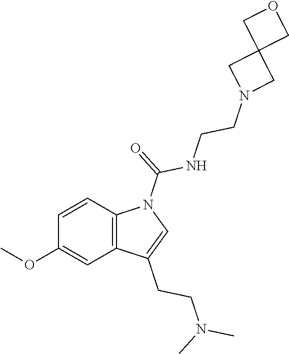 [SMILES: COc1ccc2c(c1)c(CCN(C)C)cn2C(=O)NCCN1CC2(COC2)C1] |
| | CN(CCC1═CN(C2═C1C═C(OC)C═C2)C(NCCN3CC4(C3)COC4)═O)C |
| 61 | 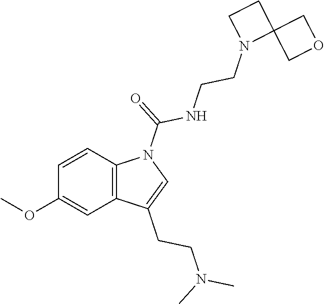 [SMILES: COc1ccc2c(c1)c(CCN(C)C)cn2C(=O)NCCN1CCC12COC2] |
| | CN(CCC1═CN(C2═C1C═C(OC)C═C2)C(NCCN3CCC34COC4)═O)C |
| 62 | 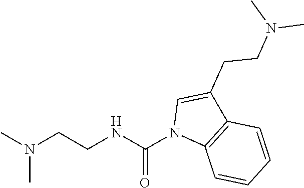 |
| | CN(CCC1═CN(C2═C1C═CC═C2)C(NCCN(C)C)═O)C |
| 63 | 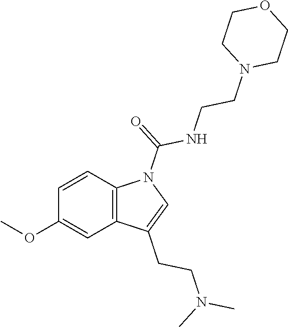 [SMILES: COc1ccc2c(c1)c(CCN(C)C)cn2C(=O)NCCN1CCOCC1] |
| | CN(CCC1═CN(C2═C1C═C(OC)C═C2)C(NCCN3CCOCC3)═O)C |
| 64 | 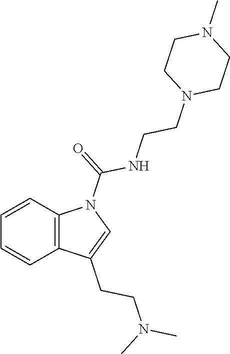 [SMILES: CN(C)CCc1cn(C(=O)NCCN2CCN(C)CC2)c2ccccc12] |
| | CN(CCC1═CN(C2═C1C═CC═C2)C(NCCN3CCN(CC3)C)═O)C |
| 65 | 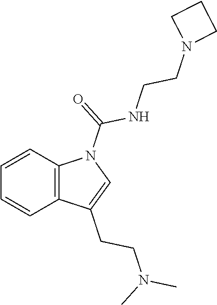 [SMILES: CN(C)CCc1cn(C(=O)NCCN2CCC2)c2ccccc12] |
| | CN(CCC1═CN(C2═C1C═CC═C2)C(NCCN3CCC3)═O)C |
| 66 | 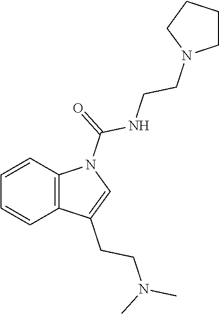 [SMILES: CN(C)CCc1cn(C(=O)NCCN2CCCC2)c2ccccc12] |
| | CN(CCC1═CN(C2═C1C═CC═C2)C(NCCN3CCC3)═O)C |
| 67 | 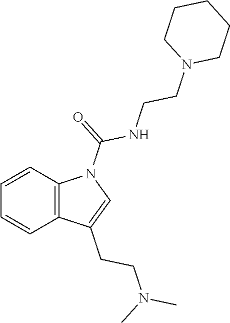 [SMILES: CN(C)CCc1cn(C(=O)NCCN2CCCCC2)c2ccccc12] |
| | CN(CCC1═CN(C2═C1C═CC═C2)C(NCCN3CCCCC3)═O)C |
| 68 | 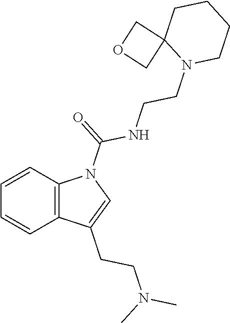 [SMILES: CN(C)CCc1cn(C(=O)NCCN2CCCCC23COC3)c2ccccc12] |
| | CN(CCC1═CN(C2═C1C═CC═C2)C(NCCN3CCCCC34COC4)═O)C |
| 69 | 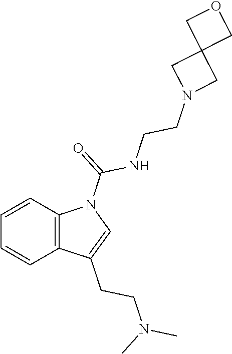 [SMILES: CN(C)CCc1cn(C(=O)NCCN2CC3(COC3)C2)c2ccccc12] |
| | CN(CCC1═CN(C2═C1C═CC═C2)C(NCCN3CC4(C3)COC4)═O)C |
| 70 | 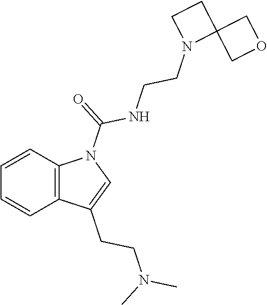 [SMILES: CN(C)CCc1cn(C(=O)NCCN2CCC23COC3)c2ccccc12] |
| | CN(CCC1═CN(C2═C1C═CC═C2)C(NCCN3CCC34COC4)═O)C |
| 71 | 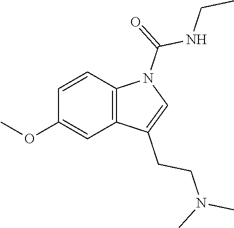 [SMILES: CCNC(=O)n1cc(CCN(C)C)c2cc(OC)ccc21] |
| | CN(CCC1═CN(C2═C1C═C(OC)C═C2)C(NCC)═O)C |
| 72 | 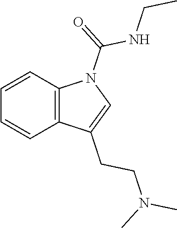 [SMILES: CCNC(=O)n1cc(CCN(C)C)c2ccccc21] |
| | CN(CCC1═CN(C2═C1C═CC═C2)C(NCC)═O)C |
| 73 | 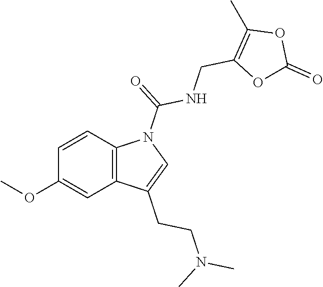 [SMILES: COc1ccc2c(c1)c(CCN(C)C)cn2C(=O)NCc1oc(=O)oc1C] |
| | CN(CCC1═CN(C2═C1C═C(OC)C═C2)C(NCC(OC3═O)═C(O3)C)═O)C |
| 74 | 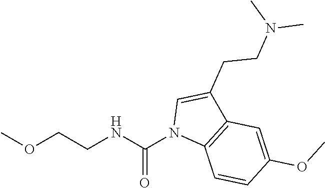 |
| | CN(CCC1═CN(C2═C1C═C(OC)C═C2)C(NCCOC)═O)C |
| 75 | 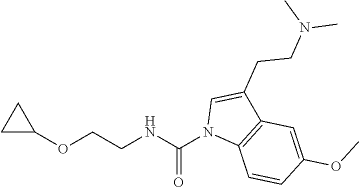 [SMILES: COc1ccc2c(c1)c(CCN(C)C)cn2C(=O)NCCOC1CC1] |
| | CN(CCC1═CN(C2═C1C═C(OC)C═C2)C(NCCOC3CC3)═O)C |
| 76 | 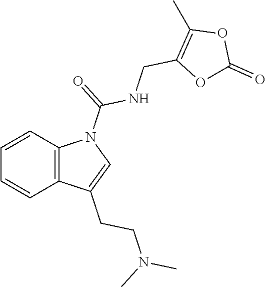 [SMILES: Cc1oc(=O)oc1CNC(=O)n1cc(CCN(C)C)c2ccccc21] |
| | CN(CCC1═CN(C2═C1C═CC═C2)C(NCC(OC3═O)═C(O3)C)═O)C |
| 77 | 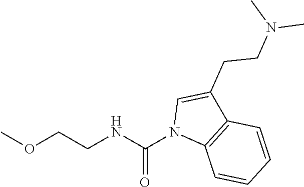 |
| | CN(CCC1═CN(C2═C1C═CC═C2)C(NCCOC)═O)C |
| 78 | 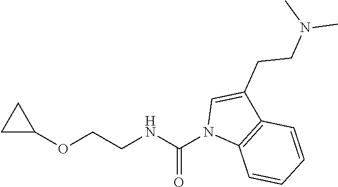 [SMILES: CN(C)CCc1cn(C(=O)NCCOC2CC2)c2ccccc12] |
| | CN(CCC1═CN(C2═C1C═CC═C2)C(NCCOC3CC3)═O)C |
| 79 | 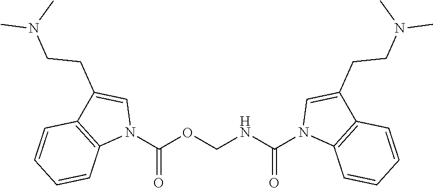 |
| | CN(CCC1═CN(C2═C1C═CC═C2)C(NCOC(N3C4═C(C(CCN(C)C)═C3)C═CC═C4)═O)═O)C |
| 80 | 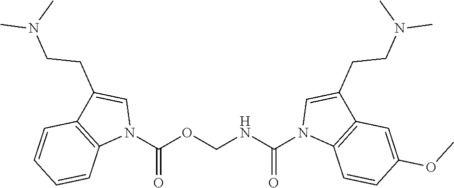 |
| | CN(CCC1═CN(C2═C1C═C(OC)C═C2)C(NCOC(N3C4═C(C(CCN(C)C)═C3)C═CC═C4)═O)═O)C |
| 81 | 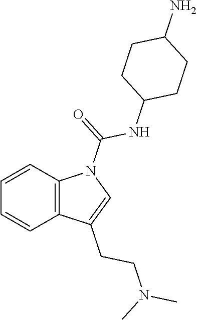 [SMILES: CN(C)CCc1cn(C(=O)NC2CCC(N)CC2)c2ccccc12] |
| | CN(CCC1═CN(C2═C1C═CC═C2)C(NC3CCC(CC3)N)═O)C |
| 82 | 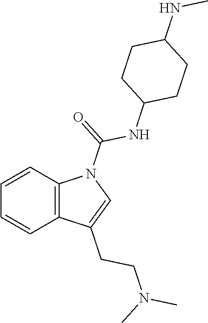 [SMILES: CNC1CCC(NC(=O)n2cc(CCN(C)C)c3ccccc32)CC1] |
| | CN(CCC1═CN(C2═C1C═CC═C2)C(NC3CCC(CC3)NC)═O)C |
| 83 | 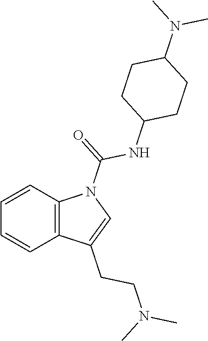 [SMILES: CN(C)CCc1cn(C(=O)NC2CCC(N(C)C)CC2)c2ccccc12] |
| | CN(CCC1═CN(C2═C1C═CC═C2)C(NC3CCC(CC3)N(C)C)═O)C |
| 84 | 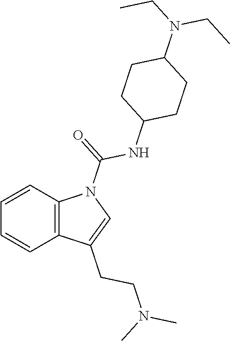 [SMILES: CCN(CC)C1CCC(NC(=O)n2cc(CCN(C)C)c3ccccc32)CC1] |
| | CN(CCC1═CN(C2═C1C═CC═C2)C(NC3CCC(CC3)N(CC)CC)═O)C |
| 85 | 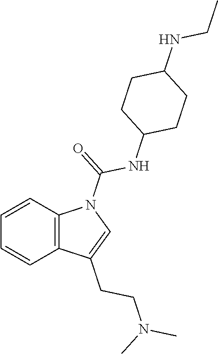 [SMILES: CCNC1CCC(NC(=O)n2cc(CCN(C)C)c3ccccc32)CC1] |
| | CN(CCC1═CN(C2═C1C═CC═C2)C(NC3CCC(CC3)NCC)═O)C |
| 86 | 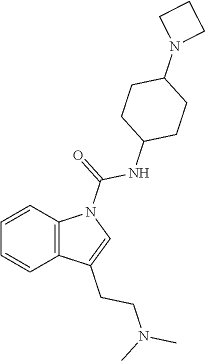 [SMILES: CN(C)CCc1cn(C(=O)NC2CCC(N3CCC3)CC2)c2ccccc12] |
| | CN(CCC1═CN(C2═C1C═CC═C2)C(NC3CCC(CC3)N4CCC4)═O)C |
| 87 | 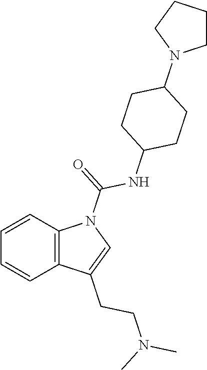 [SMILES: CN(C)CCc1cn(C(=O)NC2CCC(N3CCCC3)CC2)c2ccccc12] |
| | CN(CCC1═CN(C2═C1C═CC═C2)C(NC3CCC(CC3)N4CCCC4)═O)C |
| 88 | 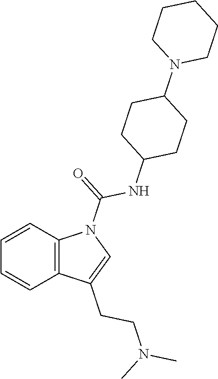 [SMILES: CN(C)CCc1cn(C(=O)NC2CCC(N3CCCCC3)CC2)c2ccccc12] |
| | CN(CCC1═CN(C2═C1C═CC═C2)C(NC3CCC(CC3)N4CCCCC4)═O)C |
| 89 | 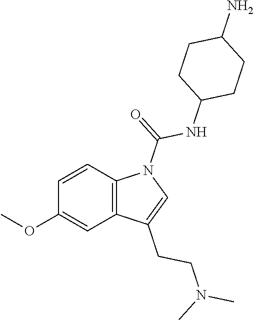 [SMILES: COc1ccc2c(c1)c(CCN(C)C)cn2C(=O)NC1CCC(N)CC1] |
| | CN(CCC1═CN(C2═C1C═C(OC)C═C2)C(NC3CCC(CC3)N)═O)C |
| 90 | 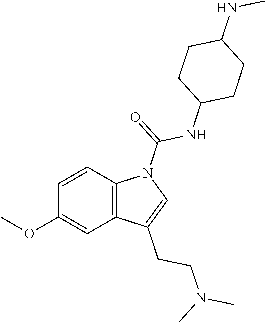 |
| | CN(CCC1═CN(C2═C1C═C(OC)C═C2)C(NC3CCC(CC3)NC)═O)C |
| 91 | 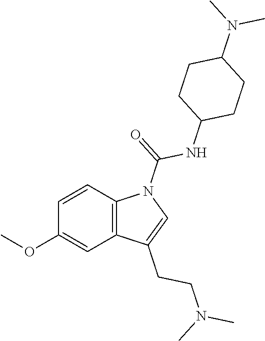 |
| | CN(CCC1═CN(C2═C1C═C(OC)C═C2)C(NC3CCC(CC3)N(C)C)═O)C |
| 92 | 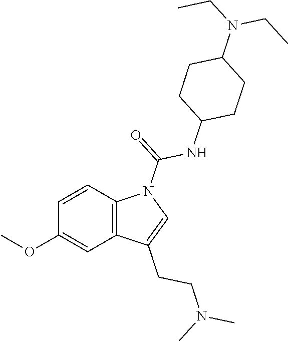 [SMILES: CCN(CC)C1CCC(NC(=O)n2cc(CCN(C)C)c3cc(OC)ccc32)CC1] |
| | CN(CCC1═CN(C2═C1C═C(OC)C═C2)C(NC3CCC(CC3)N(CC)CC)═O)C |
| 93 | 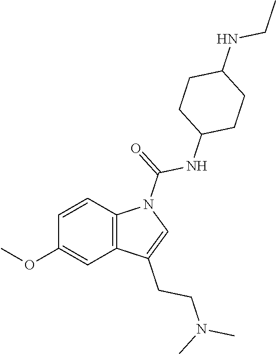 [SMILES: CCNC1CCC(NC(=O)n2cc(CCN(C)C)c3cc(OC)ccc32)CC1] |
| | CN(CCC1═CN(C2═C1C═C(OC)C═C2)(C(NC3CCC(CC3)NCC)═O)C |
| 94 | 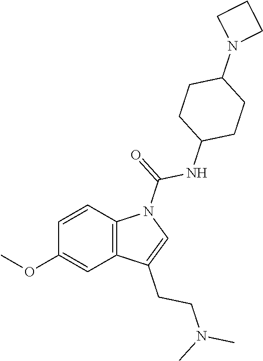 [SMILES: COc1ccc2c(c1)c(CCN(C)C)cn2C(=O)NC1CCC(N2CCC2)CC1] |
| | CN(CCC1═CN(C2═C1C═C(OC)C═C2)C(NC3CCC(CC3)N4CCC4)═O)C |
| 95 | 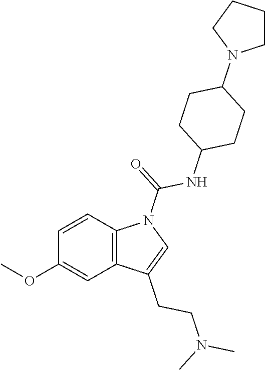 [SMILES: COc1ccc2c(c1)c(CCN(C)C)cn2C(=O)NC1CCC(N2CCCC2)CC1] |
| | CN(CCC1═CN(C2═C1C═C(OC)C═C2)C(NC3CCC(CC3)N4CCCC4)═O)C |
| 96 | 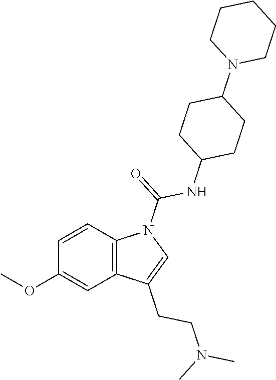 [SMILES: COc1ccc2c(c1)c(CCN(C)C)cn2C(=O)NC1CCC(N2CCCCC2)CC1] |
| | CN(CCC1═CN(C2═C1C═C(OC)C═C2)C(NC3CCC(CC3)N4CCCCC4)═O)C |
| 97 | 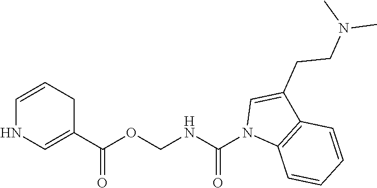 [SMILES: CN(C)CCc1cn(C(=O)NCOC(=O)C2=CNC=CC2)c2ccccc12] |
| | CN(CCC1═CN(C2═C1C═CC═C2)C(NCOC(C3═CNC═CC3)═O)═O)C |
| 98 | 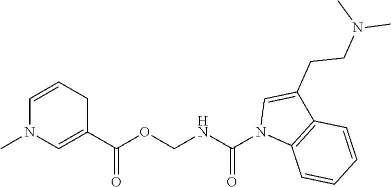 |
| | CN(CCC1═CN(C2═C1C═CC═C2)C(NCOC(C3═CN(C)C═CC3)═O)═O)C |
| 99 | 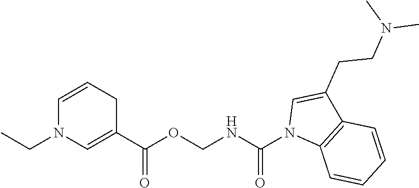 [SMILES: CCN1C=CCC(C(=O)OCNC(=O)n2cc(CCN(C)C)c3ccccc32)=C1] |
| | CN(CCC1═CN(C2═C1C═CC═C2)C(NCOC(C3═CN(C═CC3)CC═O)═O)C |
| 100 | 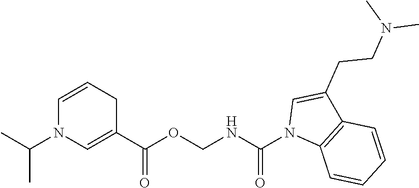 [SMILES: CC(C)N1C=CCC(C(=O)OCNC(=O)n2cc(CCN(C)C)c3ccccc32)=C1] |
| | CN(CCC1═CN(C2═C1C═CC═C2)C(NCOC(C3═CN(C═CC3)C(C)C)═O)═O)C |
| 101 | 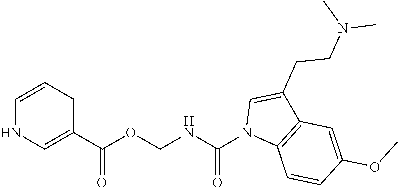 [SMILES: COc1ccc2c(c1)c(CCN(C)C)cn2C(=O)NCOC(=O)C1=CNC=CC1] |
| | CN(CCC1═CN(C2═C1C═C(OC)C═C2)C(NCOC(C3═CNC═CC3)═O)═O)C |
| 102 | 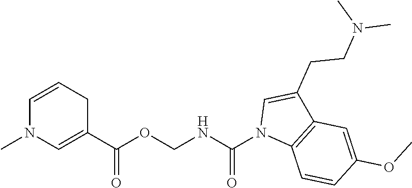 |
| | CN(CCC1═CN(C2═C1C═C(OC)C═C2)C(NCOC(C3═CN(C)C═CC3)═O)═O)C |
| 103 | 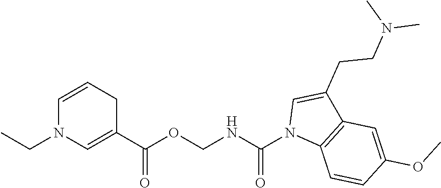 [SMILES: CCN1C=CCC(C(=O)OCNC(=O)n2cc(CCN(C)C)c3cc(OC)ccc32)=C1] |
| | CN(CCC1═CN(C2═C1C═C(OC)C═C2)C(NCOC(C3═CN(C═CC3)CC)═O)═O)C |
| 104 | 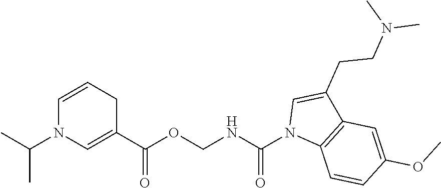 [SMILES: COc1ccc2c(c1)c(CCN(C)C)cn2C(=O)NCOC(=O)C1=CN(C(C)C)C=CC1] |
| | CN(CCC1═CN(C2═C1C═C(OC)C═C2)C(NCOC(C3═CN(C═CC3)C(C)C)═O)═O)C |
| 105 | 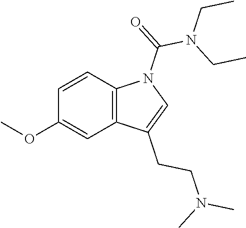 [SMILES: CCN(CC)C(=O)n1cc(CCN(C)C)c2cc(OC)ccc21] |
| | CN(CCC1═CN(C2═C1C═C(OC)C═C2)C(N(CC)CC)═O)C |
| 106 | 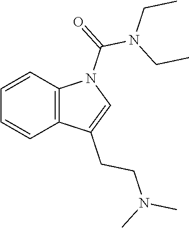 [SMILES: CCN(CC)C(=O)n1cc(CCN(C)C)c2ccccc21] |
| | CN(CCC1═CN(C2═C1C═CC═C2)C(N(CC)CC)═O)C |
| 107 | 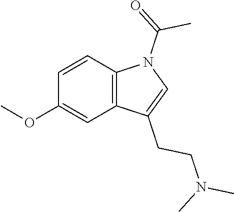 |
| | CN(CCC1═CN(C2═C1C═C(OC)C═C2)C(C)═O)C |
| 108 | 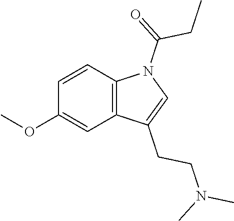 |
| | CN(CCC1═CN(C2═C1C═C(OC)C═C2)C(CC═O)C |
| 109 | 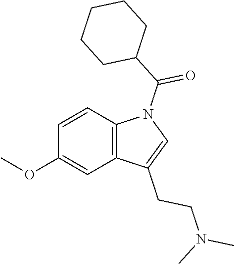 |
| | CN(CCC1═CN(C2═C1C═C(OC)C═C2)C(C3CCCCC3)═O)C |
| 110 | 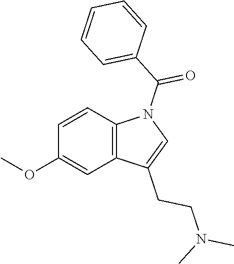 |
| | CN(CCC1═CN(C2═C1C═C(OC)C═C2)C(C3═CC═CC═C3)═O)C |
| 111 | 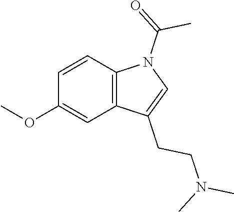 |
| | CN(CCC1═CN(C2═C1C═C(OC)C═C2)C(C)═O)C |
| 112 | 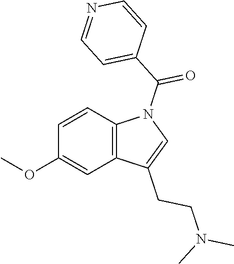 |
| | CN(CCC1═CN(C2═C1C═C(OC)C═C2)C(C3═CC═NC═C3)═O)C |
| 113 | 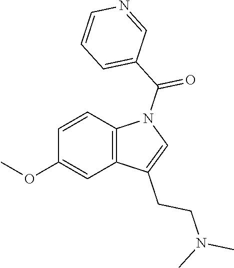 |
| | CN(CCC1═CN(C2═C1C═C(OC)C═C2)C(C3═CC═CN═C3)═O)C |
| 114 | 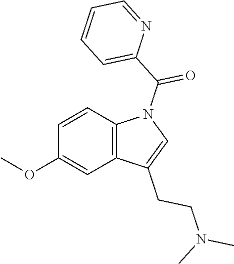 |
| | CN(CCC1═CN(C2═C1C═C(OC)C═C2)C(C3═CC═CC═N3)═O)C |
| 115 | 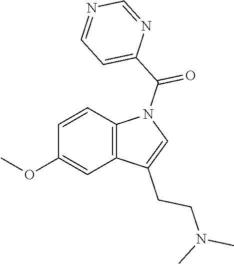 |
| | CN(CCC1═CN(C2═C1C═C(OC)C═C2)C(C3═CC═NC═N3)═O)C |
| 116 | 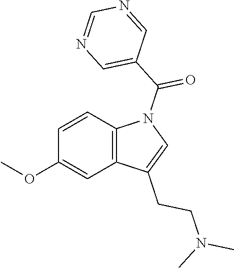 |
| | CN(CCC1═CN(C2═C1C═C(OC)C═C2)C(C3═CN═CN═C3)═O)C |
| 117 | 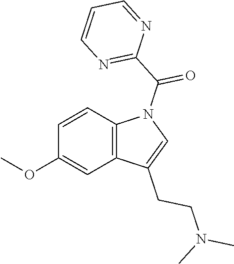 |
| | CN(CCC1═CN(C2═C1C═C(OC)C═C2)C(C3═NC═CC═N3)═O)C |
| 118 | 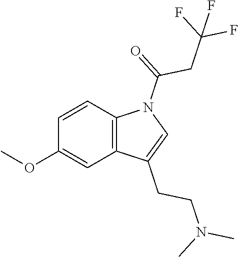 [SMILES: COc1ccc2c(c1)c(CCN(C)C)cn2C(=O)CC(F)(F)F] |
| | CN(CCC1═CN(C2═C1C═C(OC)C═C2)C(CC(F)(F)F)═O)C |
| 119 | 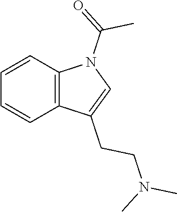 [SMILES: CC(=O)n1cc(CCN(C)C)c2ccccc21] |
| | CN(CCC1═CN(C2═C1C═CC═C2)C(C)═O)C |
| 120 | 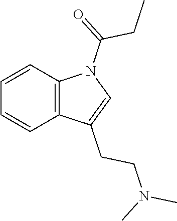 [SMILES: CCC(=O)n1cc(CCN(C)C)c2ccccc21] |
| | CN(CCC1═CN(C2═C1C═CC═C2)C(CC)═O)C |
| 121 | 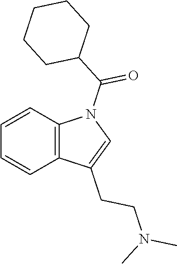 [SMILES: CN(C)CCc1cn(C(=O)C2CCCCC2)c2ccccc12] |
| | CN(CCC1═CN(C2═C1C═CC═C2)C(C3CCCCC3)═O)C |
| 122 | 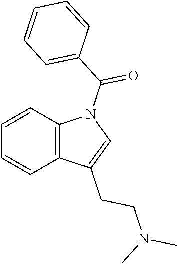 [SMILES: CN(C)CCc1cn(C(=O)c2ccccc2)c2ccccc12] |
| | CN(CCC1═CN(C2═C1C═CC═C2)C(C3═CC═CC═C3)═O)C |
| 123 | 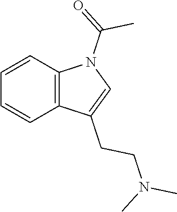 [SMILES: CC(=O)n1cc(CCN(C)C)c2ccccc21] |
| | CN(CCC1═CN(C2═C1C═CC═C2)C(C)═O)C |
| 124 | 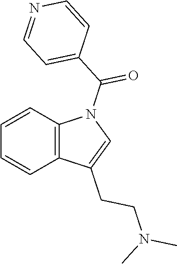 [SMILES: CN(C)CCc1cn(C(=O)c2ccncc2)c2ccccc12] |
| | CN(CCC1═CN(C2═C1C═CC═C2)C(C3═CC═NC═C3)═O)C |
| 125 | 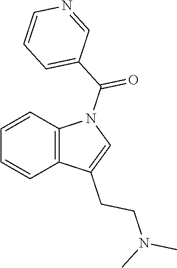 [SMILES: CN(C)CCc1cn(C(=O)c2cccnc2)c2ccccc12] |
| | CN(CCC1═CN(C2═C1C═CC═C2)C(C3═CC═CN═C3)═O)C |
| 126 | 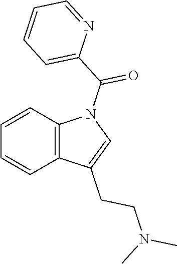 [SMILES: CN(C)CCc1cn(C(=O)c2ccccn2)c2ccccc12] |
| | CN(CCC1═CN(C2═C1C═CC═C2)C(C3═CC═CC═N3)═O)C |
| 127 | 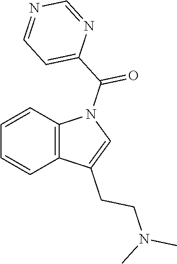 [SMILES: CN(C)CCc1cn(C(=O)c2ccncn2)c2ccccc12] |
| | CN(CCC1═CN(C2═C1C═CC═C2)C(C3═CC═NC═N3)═O)C |
| 128 | 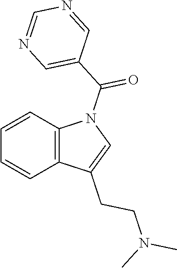 [SMILES: CN(C)CCc1cn(C(=O)c2cncnc2)c2ccccc12] |
| | CN(CCC1═CN(C2═C1C═CC═C2)C(C3═CN═CN═C3)═O)C |
| 129 | 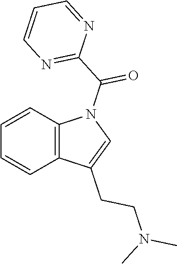 [SMILES: CN(C)CCc1cn(C(=O)c2ncccn2)c2ccccc12] |
| | CN(CCC1═CN(C2═C1C═CC═C2)C(C3═NC═CC═N3)═O)C |
| 130 | 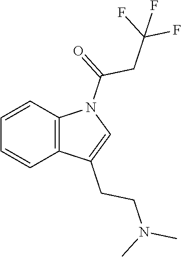 [SMILES: CN(C)CCc1cn(C(=O)CC(F)(F)F)c2ccccc12] |
| | CN(CCC1═CN(C2═C1C═CC═C2)C(CC(F)(F)F)═O)C |
| 131 | 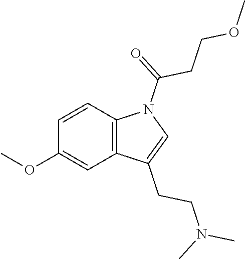 |
| | CN(CCC1═CN(C2═C1C═C(OC)C═C2)C(CCOC)═O)C |
| 132 | 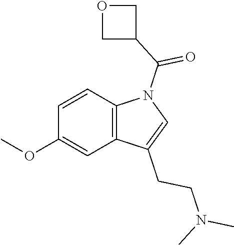 |
| | CN(CCC1═CN(C2═C1C═C(OC)C═C2)C(C3COC3)═O)C |
| 133 | 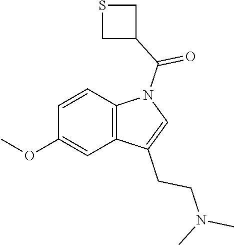 |
| | CN(CCC1═CN(C2═C1C═C(OC)C═C2)C(C3CSC3)═O)C |
| 134 | 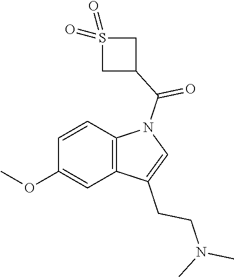 |
| | CN(CCC1═CN(C2═C1C═C(OC)C═C2)C(C3CS(C3)(═O)═O)═O)C |
| 135 | 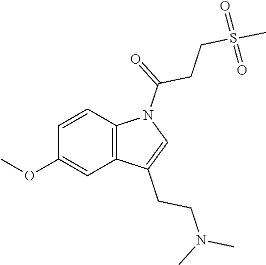 |
| | CN(CCC1═CN(C2═C1C═C(OC)C═C2)C(CCS(═O)(C)═O)═O)C |
| 136 | 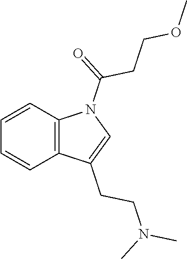 [SMILES: COCCC(=O)n1cc(CCN(C)C)c2ccccc21] |
| | CN(CCC1═CN(C2═C1C═CC═C2)C(CCOC)═O)C |
| 137 | 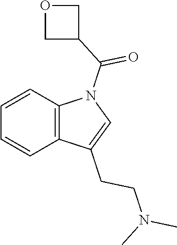 [SMILES: CN(C)CCc1cn(C(=O)C2COC2)c2ccccc12] |
| | CN(CCC1═CN(C2═C1C═CC═C2)C(C3COC3)═O)C |
| 138 | 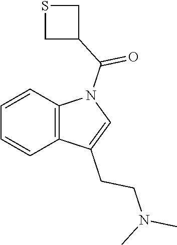 [SMILES: CN(C)CCc1cn(C(=O)C2CSC2)c2ccccc12] |
| | CN(CCC1═CN(C2═C1C═CC═C2)C(C3CSC3)═O)C |
| 139 | 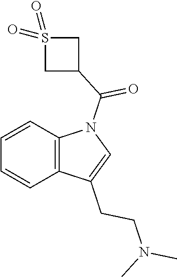 [SMILES: CN(C)CCc1cn(C(=O)C2CS(=O)(=O)C2)c2ccccc12] |
| | CN(CCC1═CN(C2═C1C═CC═C2)C(C3CS(C3)(═O)═O)═O)C |
| 140 | 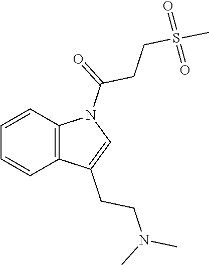 [SMILES: CN(C)CCc1cn(C(=O)CCS(C)(=O)=O)c2ccccc12] |
| | CN(CCC1═CN(C2═C1C═CC═C2)C(CCS(═O)(C)═O)═O)C |
| 141 | 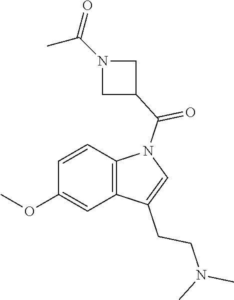 |
| | CN(CCC1═CN(C2═C1C═C(OC)C═C2)C(C3CN(C3)C(C)═O)═O)C |
| 142 | 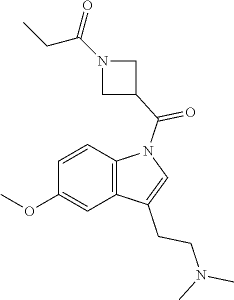 |
| | CN(CCC1═CN(C2═C1C═C(OC)C═C2)C(C3CN(C3)C(CC)═O)═O)C |
| 143 | 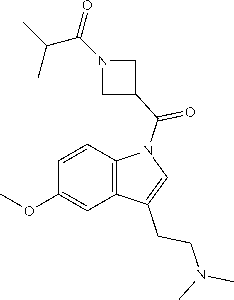 |
| | CN(CCC1═CN(C2═C1C═C(OC)C═C2)C(C3CN(C3)C(C(C)C)═O)═O)C |
| 144 | 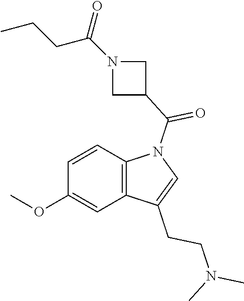 [SMILES: CCCC(=O)N1CC(C(=O)n2cc(CCN(C)C)c3cc(OC)ccc32)C1] |
| | CN(CCC1═CN(C2═C1C═C(OC)C═C2)C(C3CN(C3)C(CCC)═O)═O)C |
| 145 | 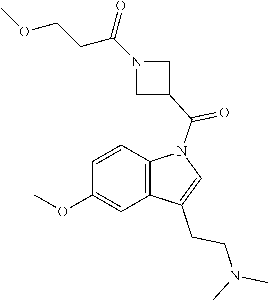 |
| | CN(CCC1═CN(C2═C1C═C(OC)C═C2)C(C3CN(C3)C(CCOC)═O)═O)C |
| 146 | 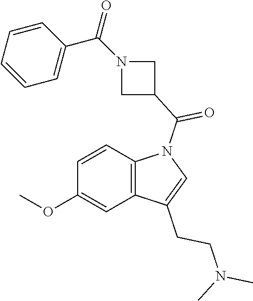 [SMILES: COc1ccc2c(c1)c(CCN(C)C)cn2C(=O)C1CN(C(=O)c2ccccc2)C1] |
| | CN(CCC1═CN(C2═C1C═C(OC)C═C2)C(C3CN(C3)C(C4═CC═CC═C4)═O)═O)C |
| 147 | 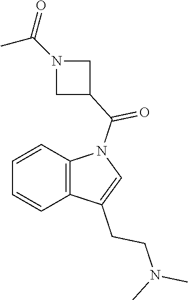 [SMILES: CC(=O)N1CC(C(=O)n2cc(CCN(C)C)c3ccccc32)C1] |
| | CN(CCC1═CN(C2═C1C═CC═C2)C(C3CN(C3)C(C)═O)═O)C |
| 148 | 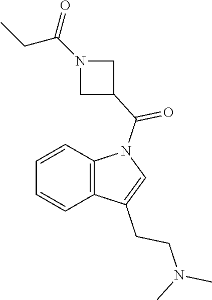 [SMILES: CCC(=O)N1CC(C(=O)n2cc(CCN(C)C)c3ccccc32)C1] |
| | CN(CCC1═CN(C2═C1C═CC═C2)C(C3CN(C3)C(CC)═O)═O)C |
| 149 | 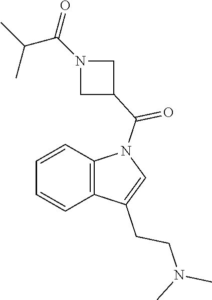 [SMILES: CC(C)C(=O)N1CC(C(=O)n2cc(CCN(C)C)c3ccccc32)C1] |
| | CN(CCC1═CN(C2═C1C═CC═C2)C(C3CN(C3)C(C(C)C)═O)═O)C |
| 150 | 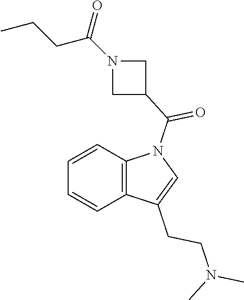 [SMILES: CCCC(=O)N1CC(C(=O)n2cc(CCN(C)C)c3ccccc32)C1] |
| | CN(CCC1═CN(C2═C1C═CC═C2)C(C3CN(C3)C(CCC)═O)═O)C |
| 151 | 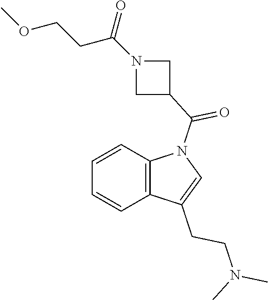 |
| | CN(CCC1═CN(C2═C1C═CC═C2)C(C3CN(C3)C(CCOC)═O)═O)C |
| 152 | 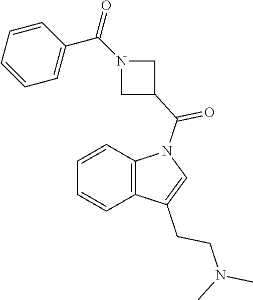 [SMILES: CN(C)CCc1cn(C(=O)C2CN(C(=O)c3ccccc3)C2)c2ccccc12] |
| | CN(CCC1═CN(C2═C1C═CC═C2)C(C3CN(C3)C(C4═CC═CC═C4)═O)═O)C |
| 153 | 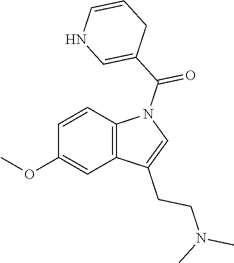 |
| | CN(CCC1═CN(C2═C1C═C(OC)C═C2)C(C3═CNC═CC3)═O)C |
| 154 | 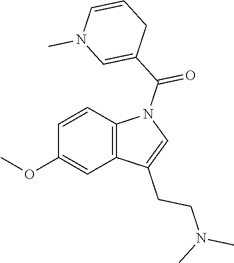 |
| | CN(CCC1═CN(C2═C1C═C(OC)C═C2)C(C3═CN(C)C═CC3)═O)C |
| 155 | 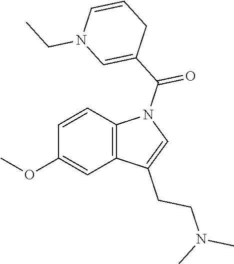 |
| | CN(CCC1═CN(C2═C1C═C(OC)C═C2)C(C3═CN(C═CC3)CC)═O)C |
| 156 | 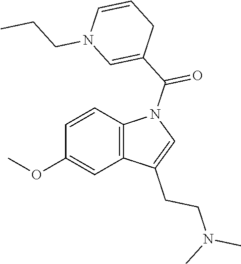 [SMILES: CCCN1C=CCC(C(=O)n2cc(CCN(C)C)c3cc(OC)ccc32)=C1] |
| | CN(CCC1═CN(C2═C1C═C(OC)C═C2)C(C3═CN(C═CC3)CCC)═O)C |
| 157 | 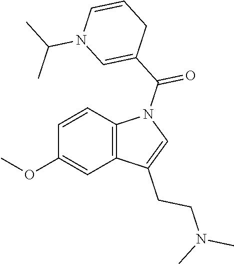 |
| | CN(CCC1═CN(C2═C1C═C(OC)C═C2)C(C3═CN(C═CC3)C(C)C)═O)C |
| 158 | 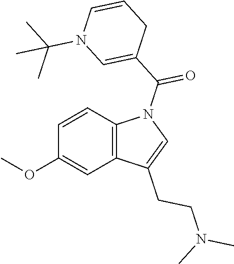 |
| | CN(CCC1═CN(C2═C1C═C(OC)C═C2)C(C3═CN(C═CC3)C(C)(C)C)═O)C |
| 159 | 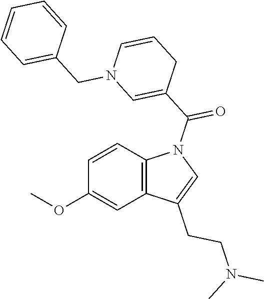 [SMILES: COc1ccc2c(c1)c(CCN(C)C)cn2C(=O)C1=CN(Cc2ccccc2)C=CC1] |
| | CN(CCC1═CN(C2═C1C═C(OC)C═C2)C(C3═CN(C═CC3)CC4═CC═CC═C4)═O)C |
| 160 | 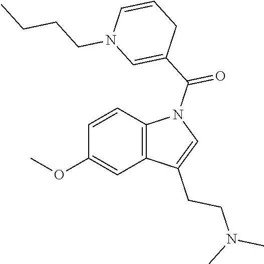 [SMILES: CCCCN1C=CCC(C(=O)n2cc(CCN(C)C)c3cc(OC)ccc32)=C1] |
| | CN(CCC1═CN(C2═C1C═C(OC)C═C2)C(C3═CN(C═CC3)CCCC)═O)C |
| 161 | 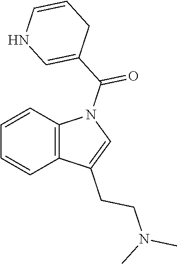 [SMILES: CN(C)CCc1cn(C(=O)C2=CNC=CC2)c2ccccc12] |
| | CN(CCC1═CN(C2═C1C═CC═C2)C(C3═CNC═CC3)═O)C |
| 162 | 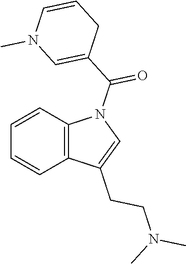 |
| | CN(CCC1═CN(C2═C1C═CC═C2)C(C3CN(C)C═CC3)═O)C |
| 163 | 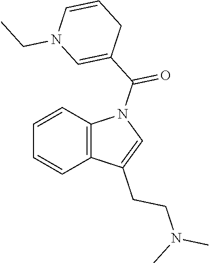 [SMILES: CCN1C=CCC(C(=O)n2cc(CCN(C)C)c3ccccc32)=C1] |
| | CN(CCC1═CN(C2═C1C═CC═C2)C(C3═CN(C═CC3)CC)═O)C |
| 164 | 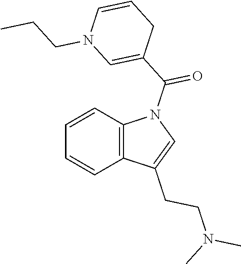 [SMILES: CCCN1C=CCC(C(=O)n2cc(CCN(C)C)c3ccccc32)=C1] |
| | CN(CCC1═CN(C2═C1C═CC═C2)C(C3═CN(C═CC3)CCC)═O)C |
| 165 | 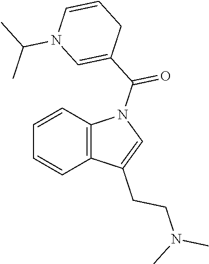 [SMILES: CC(C)N1C=CCC(C(=O)n2cc(CCN(C)C)c3ccccc32)=C1] |
| | CN(CCC1═CN(C2═C1C═CC═C2)C(C3═CN(C═CC3)C(C)C)═O)C |
| 166 | 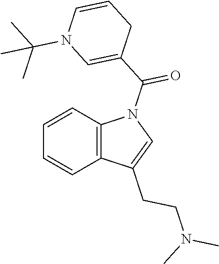 [SMILES: CN(C)CCc1cn(C(=O)C2=CN(C(C)(C)C)C=CC2)c2ccccc12] |
| | CN(CCC1═CN(C2═C1C═CC═C2)C(C3═CN(C═CC3)C(C)(C)C)═O)C |
| 167 | 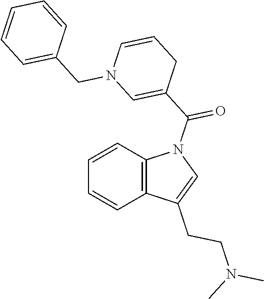 [SMILES: CN(C)CCc1cn(C(=O)C2=CN(Cc3ccccc3)C=CC2)c2ccccc12] |
| | CN(CCC1═CN(C2═C1C═CC═C2)C(C3═CN(C═CC3)CC4═CC═CC═C4)═O)C |
| 168 | 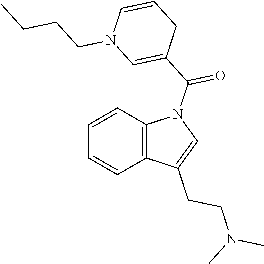 [SMILES: CCCCN1C=CCC(C(=O)n2cc(CCN(C)C)c3ccccc32)=C1] |
| | CN(CCC1═CN(C2═C1C═CC═C2)C(C3═CN(C═CC3)CCCC)═O)C |
| 169 | 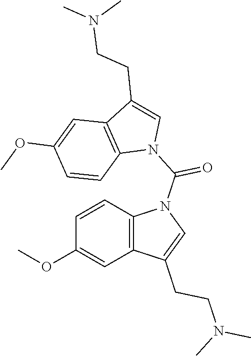 |
| | CN(CCC1═CN(C2═C1C═C(OC)C═C2)C(N3C4═C(C(CCN(C)C)═C3)C═C(OC)C═C4)═O)C |
| 170 | 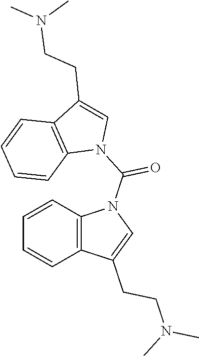 [SMILES: CN(C)CCc1cn(C(=O)n2cc(CCN(C)C)c3ccccc32)c2ccccc12] |
| | CN(CCC1═CN(C2═C1C═CC═C2)C(N3C4═C(C(CCN(C)C)═C3)C═CC═C4)═O)C |
| 171 | 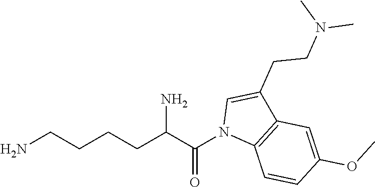 [SMILES: COc1ccc2c(c1)c(CCN(C)C)cn2C(=O)C(N)CCCCN] |
| | CN(CCC1═CN(C2═C1C═C(OC)C═C2)C(C(N)CCCCN)═O)C |
| 172 | 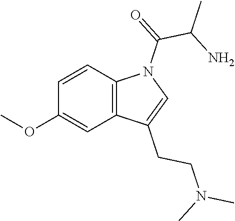 |
| | CN(CCC1═CN(C2═C1C═C(OC)C═C2)C(C(C)N)═O)C |
| 173 | 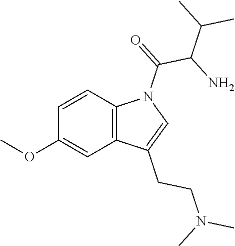 [SMILES: COc1ccc2c(c1)c(CCN(C)C)cn2C(=O)C(N)C(C)C] |
| | CN(CCC1═CN(C2═C1C═C(OC)C═C2)C(C(N)C(C)C═O)C |
| 174 | 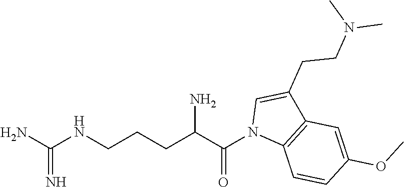 [SMILES: COc1ccc2c(c1)c(CCN(C)C)cn2C(=O)C(N)CCCNC(=N)N] |
| | CN(CCC1═CN(C2═C1C═C(OC)C═C2)C(C(N)CCCNC(N)═N)═O)C |
| 175 | 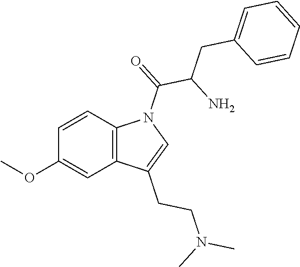 [SMILES: COc1ccc2c(c1)c(CCN(C)C)cn2C(=O)C(N)Cc1ccccc1] |
| | CN(CCC1═CN(C2═C1C═C(OC)C═C2)C(C(N)CC3═CC═CC═C3)═O)C |
| 176 | 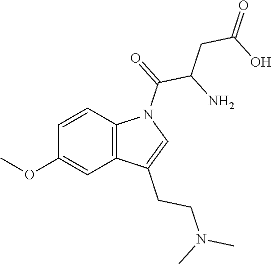 [SMILES: COc1ccc2c(c1)c(CCN(C)C)cn2C(=O)C(N)CC(=O)O] |
| | CN(CCC1═CN(C2═C1C═C(OC)C═C2)C(C(N)CC(O)═O)═O)C |
| 177 | 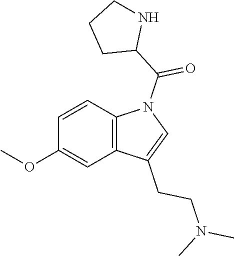 |
| | CN(CCC1═CN(C2═C1C═C(OC)C═C2)C(C3CCCN3)═O)C |
| 178 | 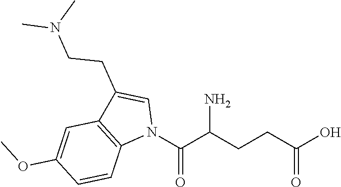 [SMILES: COc1ccc2c(c1)c(CCN(C)C)cn2C(=O)C(N)CCC(=O)O] |
| | CN(CCC1═CN(C2═C1C═C(OC)C═C2)C(C(N)CCC(O)═O)═O)C |
| 179 | 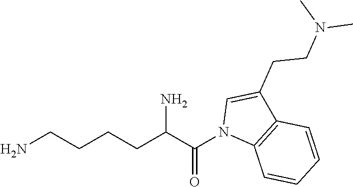 [SMILES: CN(C)CCc1cn(C(=O)C(N)CCCCN)c2ccccc12] |
| | CN(CCC1═CN(C2═C1C═CC═C2)C(C(N)CCCCN)═O)C |
| 180 | 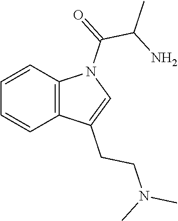 [SMILES: CC(N)C(=O)n1cc(CCN(C)C)c2ccccc21] |
| | CN(CCC1═CN(C2═C1C═CC═C2)C(C(C)N)═O)C |
| 181 | 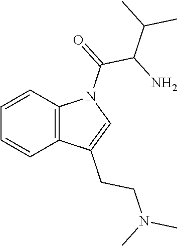 [SMILES: CC(C)C(N)C(=O)n1cc(CCN(C)C)c2ccccc21] |
| | CN(CCC1═CN(C2═C1C═CC═C2)C(C(N)C(C)C)═O)C |
| 182 | 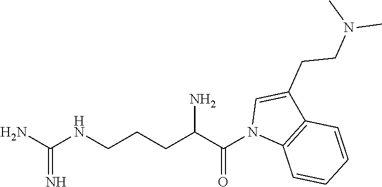 [SMILES: CN(C)CCc1cn(C(=O)C(N)CCCNC(=N)N)c2ccccc12] |
| | CN(CCC1═CN(C2═C1C═CC═C2)C(C(N)CCCNC(N)═N)═O)C |
| 183 | 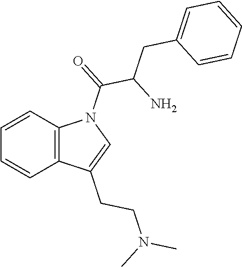 [SMILES: CN(C)CCc1cn(C(=O)C(N)Cc2ccccc2)c2ccccc12] |
| | CN(CCC1═CN(C2═C1C═CC═C2)C(C(N)CC3═CC═CC═C3)═O)C |
| 184 | 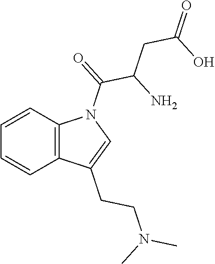 [SMILES: CN(C)CCc1cn(C(=O)C(N)CC(=O)O)c2ccccc12] |
| | CN(CCC1═CN(C2═C1C═CC═C2)C(C(N)CC(O)═O)═O)C |
| 185 | 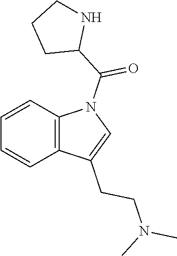 [SMILES: CN(C)CCc1cn(C(=O)C2CCCN2)c2ccccc12] |
| | CN(CCC1═CN(C2═C1C═CC═C2)C(C3CCCN3)═O)C |
| 186 | 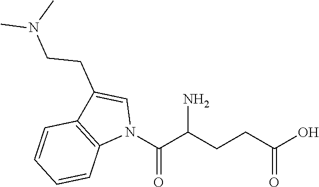 [SMILES: CN(C)CCc1cn(C(=O)C(N)CCC(=O)O)c2ccccc12] |
| | CN(CCC1═CN(C2═C1C═CC═C2)C(C(N)CCC(O)═O)═O)C |
| 187 | 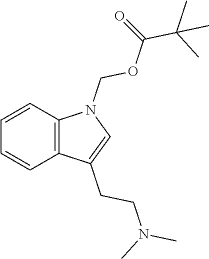 [SMILES: CN(C)CCc1cn(COC(=O)C(C)(C)C)c2ccccc12] |
| | CN(CCC1═CN(C2═C1C═CC═C2)COC(C(C)(C)C)═O)C |
| 188 | 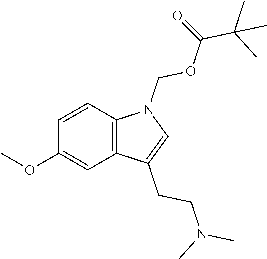 [SMILES: COc1ccc2c(c1)c(CCN(C)C)cn2COC(=O)C(C)(C)C] |
| | CN(CCC1═CN(C2═C1C═C(OC)C═C2)COC(C(C)(C)C)═O)C |
| 189 | 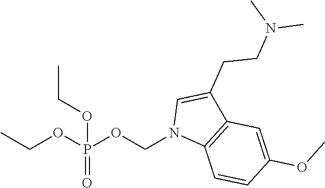 [SMILES: CCOP(=O)(OCC)OCn1cc(CCN(C)C)c2cc(OC)ccc21] |
| | CN(CCC1═CN(C2═C1C═C(OC)C═C2)COP(OCC)(OCC)═O)C |
| 190 | 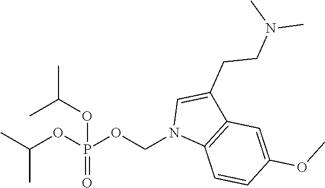 [SMILES: COc1ccc2c(c1)c(CCN(C)C)cn2COP(=O)(OC(C)C)OC(C)C] |
| | CN(CCC1═CN(C2═C1C═C(OC)C═C2)COP(OC(C)C)(OC(C)C)═O)C |
| 191 | 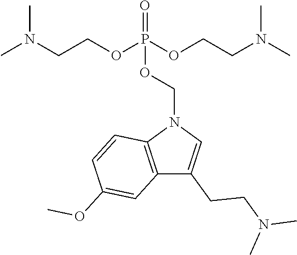 |
| | CN(CCC1═CN(C2═C1C═C(OC)C═C2)COP(OCCN(C)C)(OCCN(C)C)═O)C |
| 192 | 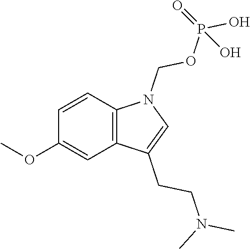 |
| | CN(CCC1═CN(C2═C1C═C(OC)C═C2)COP(O)(O)═O)C |
| 193 | 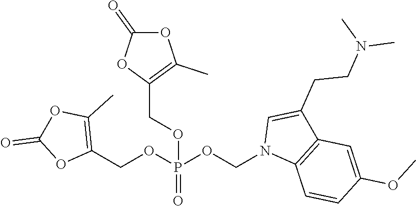 [SMILES: COc1ccc2c(c1)c(CCN(C)C)cn2COP(=O)(OCc1oc(=O)oc1C)OCc1oc(=O)oc1C] |
| | CN(CCC1═CN(C2═C1C═C(OC)C═C2)COP(OCC(OC3═O)═C(O3)C)(OCC(OC4═O)═C(O4)C)═O)C |
| 194 | 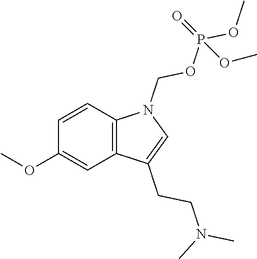 |
| | CN(CCC1═CN(C2═C1C═C(OC)C═C2)COP(OC)(OC)═O)C |
| 195 | 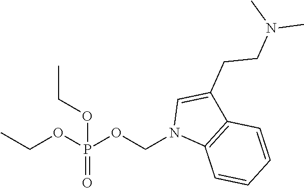 [SMILES: CCOP(=O)(OCC)OCn1cc(CCN(C)C)c2ccccc21] |
| | CN(CCC1═CN(C2═C1C═CC═C2)COP(OCC)(OCC)═O)C |
| 196 | 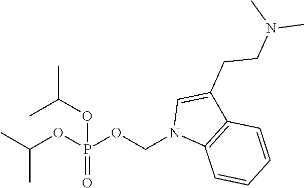 [SMILES: CC(C)OP(=O)(OCn1cc(CCN(C)C)c2ccccc21)OC(C)C] |
| | CN(CCC1═CN(C2═C1C═CC═C2)COP(OC(C)C)(OC(C)C)═O)C |
| 197 | 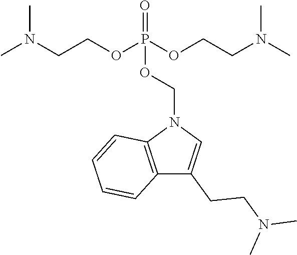 |
| | CN(CCC1═CN(C2═C1C═CC═C2)COP(OCCN(C)C)(OCCN(C)C)═O)C |
| 198 | 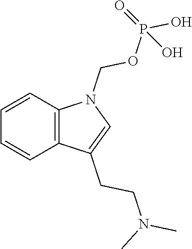 [SMILES: CN(C)CCc1cn(COP(=O)(O)O)c2ccccc12] |
| | CN(CCC1═CN(C2═C1C═CC═C2)COP(O)(O)═O)C |
| 199 | 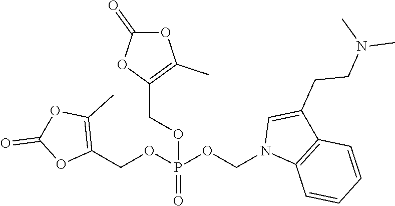 [SMILES: Cc1oc(=O)oc1COP(=O)(OCc1oc(=O)oc1C)OCn1cc(CCN(C)C)c2ccccc21] |
| | CN(CCC1═CN(C2═C1C═CC═C2)COP(OCC(OC3═O)═C(O3)C)(OCC(OC4═O)═C(O4)C)═O)C |
| 200 | 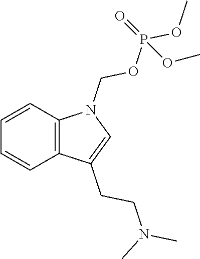 [SMILES: COP(=O)(OC)OCn1cc(CCN(C)C)c2ccccc21] |
| | CN(CCC1═CN(C2═C1C═CC═C2)COP(OC)(OC)═O)C |
| 201 | 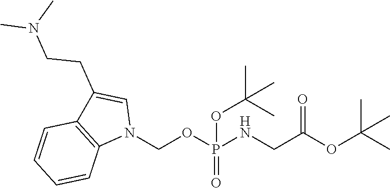 [SMILES: CN(C)CCc1cn(COP(=O)(NCC(=O)OC(C)(C)C)OC(C)(C)C)c2ccccc12] |
| | CN(CCC1═CN(C2═C1C═CC═C2)COP(OC(C)(C)C)(NCC(OC(C)(C)C)═O)═O)C |
| 202 | 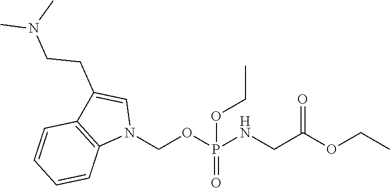 [SMILES: CCOC(=O)CNP(=O)(OCC)OCn1cc(CCN(C)C)c2ccccc21] |
| | CN(CCC1═CN(C2═C1C═CC═C2)COP(OCC)(NCC(OCC)═O)═O)C |
| 203 | 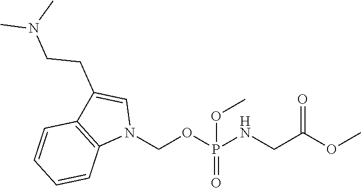 |
| | CN(CCC1═CN(C2═C1C═CC═C2)COP(OC)(NCC(OC)═O)═O)C |
| 204 | 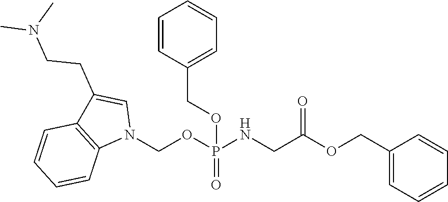 [SMILES: CN(C)CCc1cn(COP(=O)(NCC(=O)OCc2ccccc2)OCc2ccccc2)c2ccccc12] |
| | CN(CCC1═CN(C2═C1C═CC═C2)COP(OCC3═CC═CC═C3)(NCC(OCC4═CC═CC═C4)═O)═O)C |
| 205 | 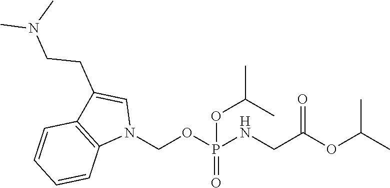 [SMILES: CC(C)OC(=O)CNP(=O)(OCn1cc(CCN(C)C)c2ccccc21)OC(C)C] |
| | CN(CCC1═CN(C2═C1C═CC═C2)COP(OC(C)C)(NCC(OC(C)C)═O)═O)C |
| 206 | 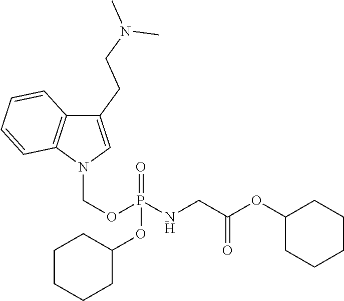 [SMILES: CN(C)CCc1cn(COP(=O)(NCC(=O)OC2CCCCC2)OC2CCCCC2)c2ccccc12] |
| | CN(CCC1═CN(C2═C1C═CC═C2)COP(OC3CCCCC3)(NCC(OC4CCCCC4)═O)═O)C |
| 207 | 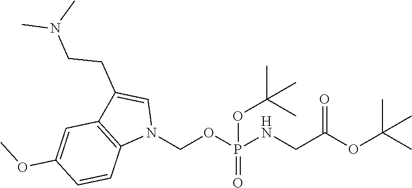 [SMILES: COc1ccc2c(c1)c(CCN(C)C)cn2COP(=O)(NCC(=O)OC(C)(C)C)OC(C)(C)C] |
| | CN(CCC1═CN(C2═C1C═C(OC)C═C2)COP(OC(C)(C)C)(NCC(OC(C)(C)C)═O)═O)C |
| 208 | 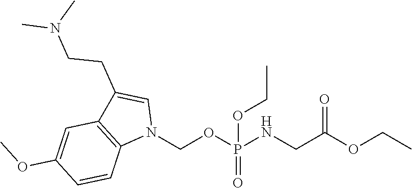 [SMILES: CCOC(=O)CNP(=O)(OCC)OCn1cc(CCN(C)C)c2cc(OC)ccc21] |
| | CN(CCC1═CN(C2═C1C═C(OC)C═C2)COP(OCC)(NCC(OCC)═O)═O)C |
| 209 | 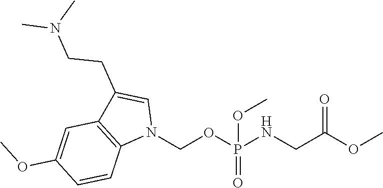 |
| | CN(CCC1═CN(C2═C1C═C(OC)C═C2)COP(OC)(NCC(OC)═O)═O)C |
| 210 | 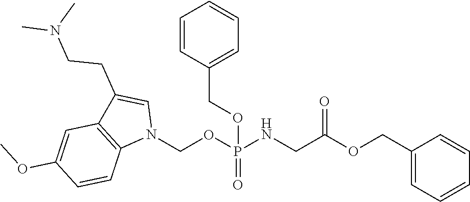 [SMILES: COc1ccc2c(c1)c(CCN(C)C)cn2COP(=O)(NCC(=O)OCc1ccccc1)OCc1ccccc1] |
| | CN(CCC1═CN(C2═C1C═C(OC)C═C2)COP(OCC3═CC═CC═C3)(NCC(OCC4═CC═CC═C4)═O)═O)C |
| 211 | 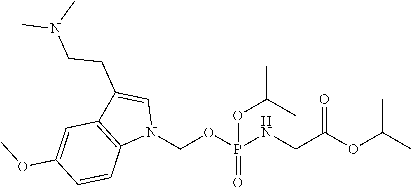 [SMILES: COc1ccc2c(c1)c(CCN(C)C)cn2COP(=O)(NCC(=O)OC(C)C)OC(C)C] |
| | CN(CCC1═CN(C2═C1C═C(OC)C═C2)COP(OC(C)C)(NCC(OC(C)C)═O)═O)C |
| 212 | 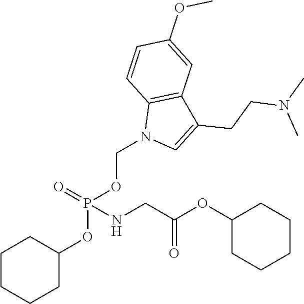 [SMILES: COc1ccc2c(c1)c(CCN(C)C)cn2COP(=O)(NCC(=O)OC1CCCCC1)OC1CCCCC1] |
| | CN(CCC1═CN(C2═C1C═C(OC)C═C2)COP(OC3CCCCC3)(NCC(OC4CCCCC4)═O)═O)C |
| 213 | 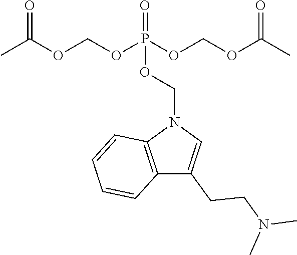 [SMILES: CC(=O)OCOP(=O)(OCOC(C)=O)OCn1cc(CCN(C)C)c2ccccc21] |
| | CN(CCC1═CN(C2═C1C═CC═C2)COP(OCOC(C)═O)(OCOC(C)═O)═O)C |
| 214 | 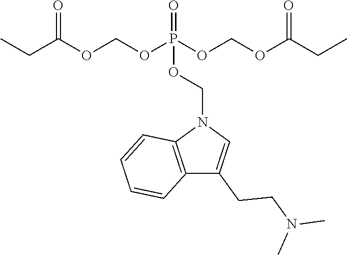 [SMILES: CCC(=O)OCOP(=O)(OCOC(=O)CC)OCn1cc(CCN(C)C)c2ccccc21] |
| | CN(CCC1═CN(C2═C1C═CC═C2)COP(OCOC(CC)═O)(OCOC(CC)═O)═O)C |
| 215 | 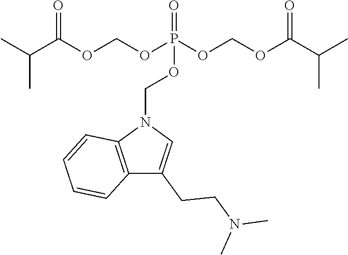 [SMILES: CC(C)C(=O)OCOP(=O)(OCOC(=O)C(C)C)OCn1cc(CCN(C)C)c2ccccc21] |
| | CN(CCC1═CN(C2═C1C═CC═C2)COP(OCOC(C(C)C)═O)(OCOC(C(C)C)═O)═O)C |
| 216 | 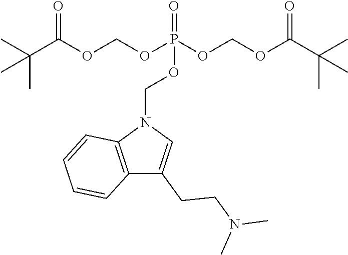 [SMILES: CN(C)CCc1cn(COP(=O)(OCOC(=O)C(C)(C)C)OCOC(=O)C(C)(C)C)c2ccccc12] |
| | CN(CCC1═CN(C2═C1C═CC═C2)COP(OCOC(C(C)(C)C)═O)(OCOC(C(C)(C)C)═O)═O)C |
| 217 | 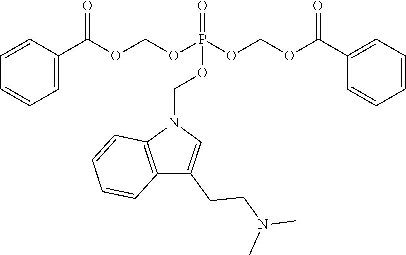 [SMILES: CN(C)CCc1cn(COP(=O)(OCOC(=O)c2ccccc2)OCOC(=O)c2ccccc2)c2ccccc12] |
| | CN(CCC1═CN(C2═C1C═CC═C2)COP(OCOC(C3═CC═CC═C3)═O)(OCOC(C4═CC═CC═C4)═O)═O)C |
| 218 | 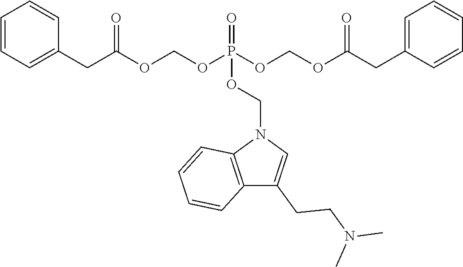 [SMILES: CN(C)CCc1cn(COP(=O)(OCOC(=O)Cc2ccccc2)OCOC(=O)Cc2ccccc2)c2ccccc12] |
| | CN(CCC1═CN(C2═C1C═CC═C2)COP(OCOC(CC3═CC═CC═C3)═O)(OCOC(CC4═CC═CC═C4)═O)═O)C |
| 219 | 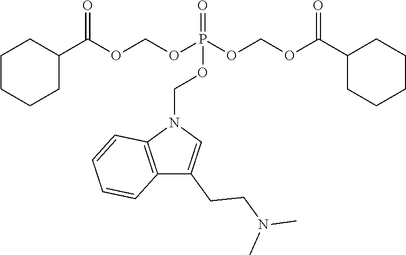 [SMILES: CN(C)CCc1cn(COP(=O)(OCOC(=O)C2CCCCC2)OCOC(=O)C2CCCCC2)c2ccccc12] |
| | CN(CCC1═CN(C2═C1C═CC═C2)COP(OCOC(C3CCCCC3)═O)(OCOC(C4CCCCC4)═O)═O)C |
| 220 | 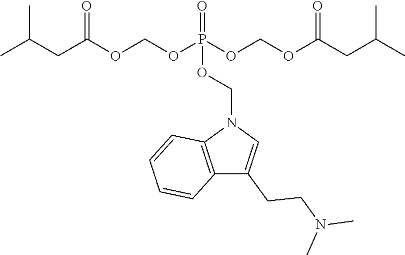 [SMILES: CC(C)CC(=O)OCOP(=O)(OCOC(=O)CC(C)C)OCn1cc(CCN(C)C)c2ccccc21] |
| | CN(CCC1═CN(C2═C1C═CC═C2)COP(OCOC(CC(C)C)═O)(OCOC(CC(C)C)═O)═O)C |
| 221 | 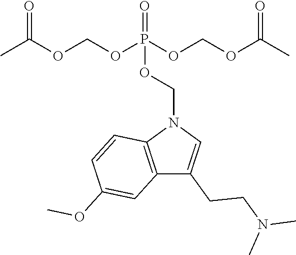 [SMILES: COc1ccc2c(c1)c(CCN(C)C)cn2COP(=O)(OCOC(C)=O)OCOC(C)=O] |
| | CN(CCC1═CN(C2═C1C═C(OC)C═C2)COP(OCOC(C)═O)(OCOC(C)═O)═O)C |
| 222 | 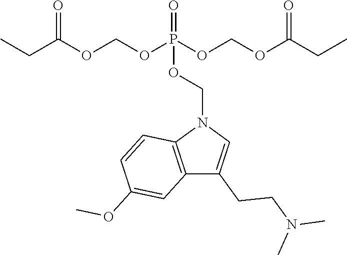 [SMILES: CCC(=O)OCOP(=O)(OCOC(=O)CC)OCn1cc(CCN(C)C)c2cc(OC)ccc21] |
| | CN(CCC1═CN(C2═C1C═C(OC)C═C2)COP(OCOC(CC)═O)(OCOC(CC)═O)═O)C |
| 223 | 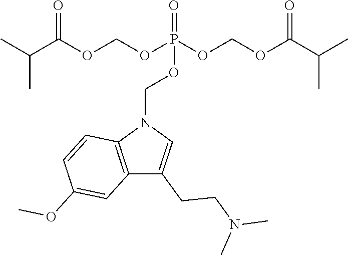 [SMILES: COc1ccc2c(c1)c(CCN(C)C)cn2COP(=O)(OCOC(=O)C(C)C)OCOC(=O)C(C)C] |
| | CN(CCC1═CN(C2═C1C═C(OC)C═C2)COP(OCOC(C(C)C)═O)(OCOC(C(C)C)═O)═O)C |
| 224 | 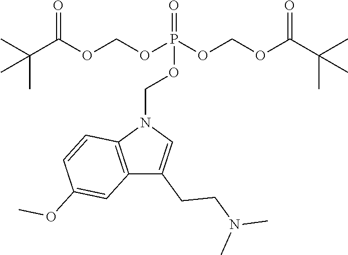 [SMILES: COc1ccc2c(c1)c(CCN(C)C)cn2COP(=O)(OCOC(=O)C(C)(C)C)OCOC(=O)C(C)(C)C] |
| | CN(CCC1═CN(C2═C1C═C(OC)C═C2)COP(OCOC(C(C)(C)C)═O)(OCOC(C(C)(C)C)═O)═O)C |
| 225 | 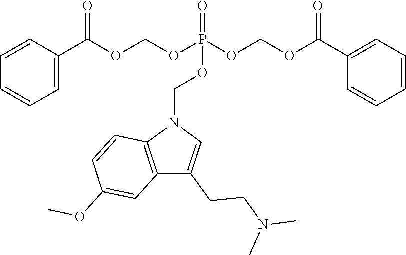 [SMILES: COc1ccc2c(c1)c(CCN(C)C)cn2COP(=O)(OCOC(=O)c1ccccc1)OCOC(=O)c1ccccc1] |
| | CN(CCC1═CN(C2═C1C═C(OC)C═C2)COP(OCOC(C3═CC═CC═C3)═O)(OCOC(C4═CC═CC═C4)═O)═O)C |
| 226 | 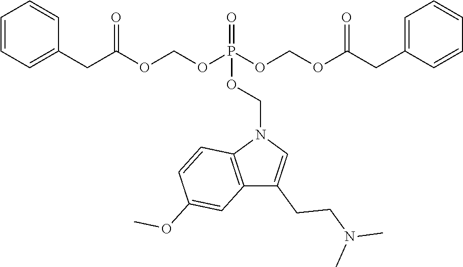 [SMILES: COc1ccc2c(c1)c(CCN(C)C)cn2COP(=O)(OCOC(=O)Cc1ccccc1)OCOC(=O)Cc1ccccc1] |
| | CN(CCC1═CN(C2═C1C═C(OC)C═C2)COP(OCOC(CC3═CC═CC═C3)═O)(OCOC(CC4═CC═CC═C4)═O)═O)C |
| 227 | 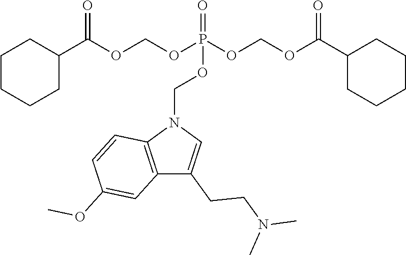 [SMILES: COc1ccc2c(c1)c(CCN(C)C)cn2COP(=O)(OCOC(=O)C1CCCCC1)OCOC(=O)C1CCCCC1] |
| | CN(CCC1═CN(C2═C1C═C(OC)C═C2)COP(OCOC(C3CCCCC3)═O)(OCOC(C4CCCCC4)═O)═O)C |
| 228 | 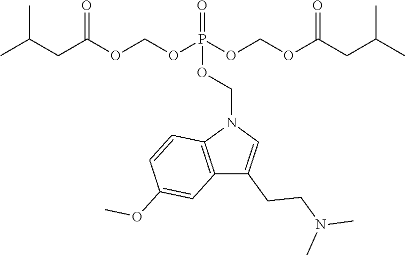 [SMILES: COc1ccc2c(c1)c(CCN(C)C)cn2COP(=O)(OCOC(=O)CC(C)C)OCOC(=O)CC(C)C] |
| | CN(CCC1═CN(C2═C1C═C(OC)C═C2)COP(OCOC(CC(C)C)═O)(OCOC(CC(C)C)═O)═O)C |
| 229 | 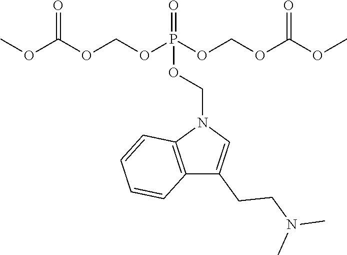 |
| | CN(CCC1-CN(C2═C1C-CC-C2)COP(OCOC(OC)═O)(OCOC(OC)═O)═O)C |
| 230 | 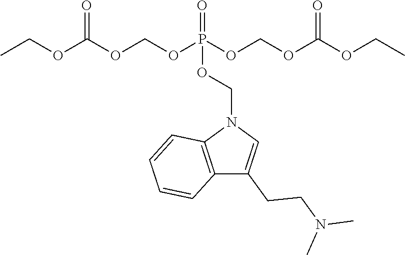 [SMILES: CCOC(=O)OCOP(=O)(OCOC(=O)OCC)OCn1cc(CCN(C)C)c2ccccc21] |
| | CN(CCC1═CN(C2═C1C═CC═C2)COP(OCOC(OCC)═O)(OCOC(OCC)═O)═O)C |
| 231 | 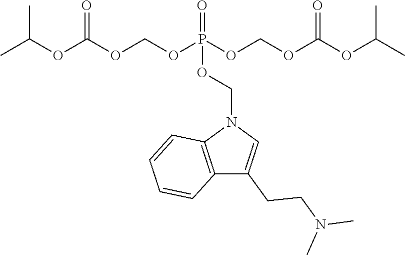 [SMILES: CC(C)OC(=O)OCOP(=O)(OCOC(=O)OC(C)C)OCn1cc(CCN(C)C)c2ccccc21] |
| | CN(CCC1═CN(C2═C1C═CC═C2)COP(OCOC(OC(C)C)═O)(OCOC(OC(C)C)═O)═O)C |
| 232 | 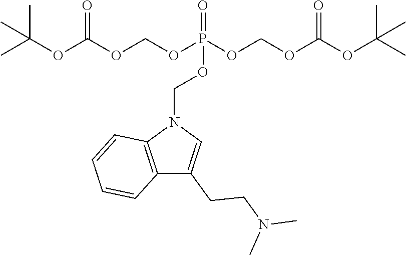 [SMILES: CN(C)CCc1cn(COP(=O)(OCOC(=O)OC(C)(C)C)OCOC(=O)OC(C)(C)C)c2ccccc12] |
| | CN(CCC1═CN(C2═C1C═CC═C2)COP(OCOC(OC(C)(C)C)═O)(OCOC(OC(C)(C)C)═O)═O)C |
| 233 | 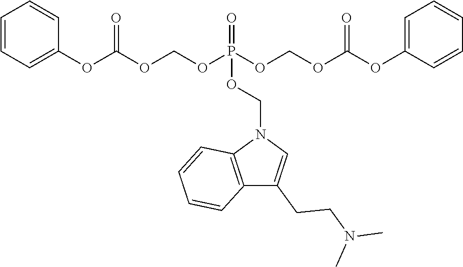 [SMILES: CN(C)CCc1cn(COP(=O)(OCOC(=O)Oc2ccccc2)OCOC(=O)Oc2ccccc2)c2ccccc12] |
| | CN(CCC1═CN(C2═C1C═CC═C2)COP(OCOC(OC3═CC═CC═C3)═O)(OCOC(OC4═CC═CC═C4)═O)═O)C |
| 234 | 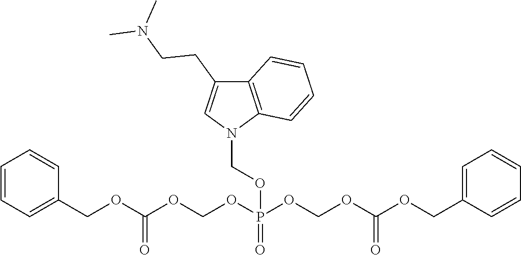 [SMILES: CN(C)CCc1cn(COP(=O)(OCOC(=O)OCc2ccccc2)OCOC(=O)OCc2ccccc2)c2ccccc12] |
| | CN(CCC1═CN(C2═C1C═CC═C2)COP(OCOC(OCC3═CC═CC═C3)═O)(OCOC(OCC4═CC═CC═C4)═O)═O)C |
| 235 | 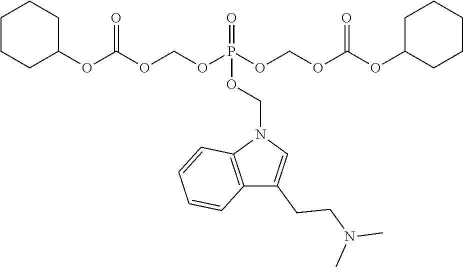 [SMILES: CN(C)CCc1cn(COP(=O)(OCOC(=O)OC2CCCCC2)OCOC(=O)OC2CCCCC2)c2ccccc12] |
| | CN(CCC1═CN(C2═C1C═CC═C2)COP(OCOC(OC3CCCCC3)═O)(OCOC(OC4CCCCC4)═O)═O)C |
| 236 | 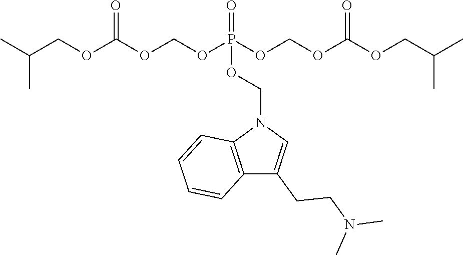 [SMILES: CC(C)COC(=O)OCOP(=O)(OCOC(=O)OCC(C)C)OCn1cc(CCN(C)C)c2ccccc21] |
| | CN(CCC1═CN(C2═C1C═CC═C2)COP(OCOC(OCC(C)C)═O)(OCOC(OCC(C)C)═O)═O)C |
| 237 | 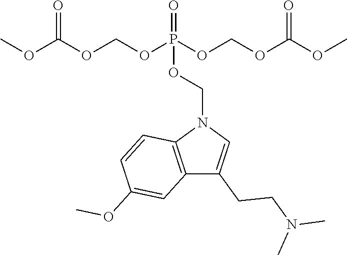 |
| | CN(CCC1═CN(C2═C1C═C(OC)C═C2)COP(OCOC(OC)═O)(OCOC(OC)═O)═O)C |
| 238 | 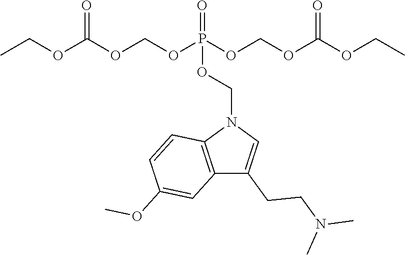 [SMILES: CCOC(=O)OCOP(=O)(OCOC(=O)OCC)OCn1cc(CCN(C)C)c2cc(OC)ccc21] |
| | CN(CCC1═CN(C2═C1C═C(OC)C═C2)COP(OCOC(OCC)═O)(OCOC(OCC)═O)═O)C |
| 239 | 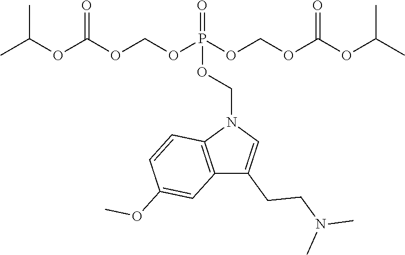 [SMILES: COc1ccc2c(c1)c(CCN(C)C)cn2COP(=O)(OCOC(=O)OC(C)C)OCOC(=O)OC(C)C] |
| | CN(CCC1═CN(C2═C1C═C(OC)C═C2)COP(OCOC(OC(C)C)═O)(OCOC(OC(C)C)═O)═O)C |
| 240 | 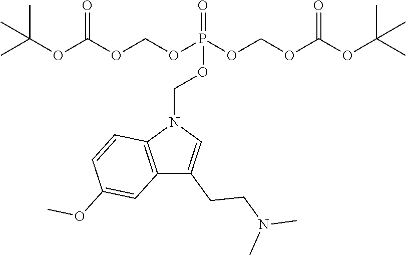 [SMILES: COc1ccc2c(c1)c(CCN(C)C)cn2COP(=O)(OCOC(=O)OC(C)(C)C)OCOC(=O)OC(C)(C)C] |
| | CN(CCC1═CN(C2═C1C═C(OC)C═C2)COP(OCOC(OC(C)(C)C)═O)(OCOC(OC(C)(C)C)═O)═O)C |
| 241 | 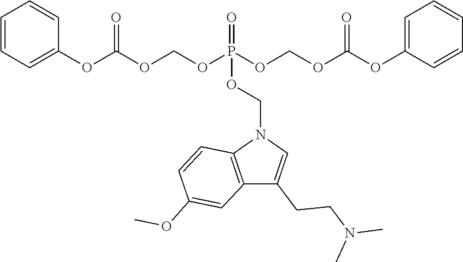 [SMILES: COc1ccc2c(c1)c(CCN(C)C)cn2COP(=O)(OCOC(=O)Oc1ccccc1)OCOC(=O)Oc1ccccc1] |
| | CN(CCC1═CN(C2═C1C═C(OC)C═C2)COP(OCOC(OC3═CC═CC═C3)═O)(OCOC(OC4═CC═CC═C4)═O)═O)C |
| 242 | 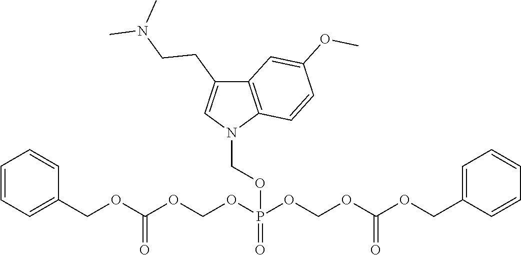 [SMILES: COc1ccc2c(c1)c(CCN(C)C)cn2COP(=O)(OCOC(=O)OCc1ccccc1)OCOC(=O)OCc1ccccc1] |
| | CN(CCC1═CN(C2═C1C═C(OC)C═C2)COP(OCOC(OCC3═CC═CC═C3)═O)(OCOC(OCC4═CC═CC═C4)═O)═O)C |
| 243 | 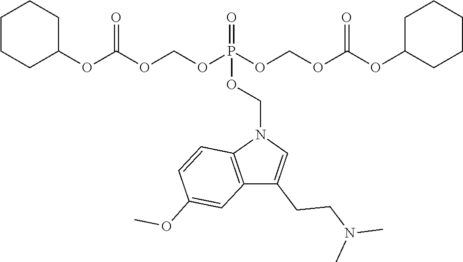 [SMILES: COc1ccc2c(c1)c(CCN(C)C)cn2COP(=O)(OCOC(=O)OC1CCCCC1)OCOC(=O)OC1CCCCC1] |
| | CN(CCC1═CN(C2═C1C═C(OC)C═C2)COP(OCOC(OC3CCCCC3)═O)(OCOC(OC4CCCCC4)═O)═O)C |
| 244 | 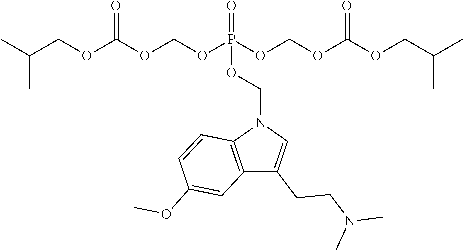 [SMILES: COc1ccc2c(c1)c(CCN(C)C)cn2COP(=O)(OCOC(=O)OCC(C)C)OCOC(=O)OCC(C)C] |
| | CN(CCC1═CN(C2═C1C═C(OC)C═C2)COP(OCOC(OCC(C)C)═O)(OCOC(OCC(C)C)═O)═O)C |
| 245 | 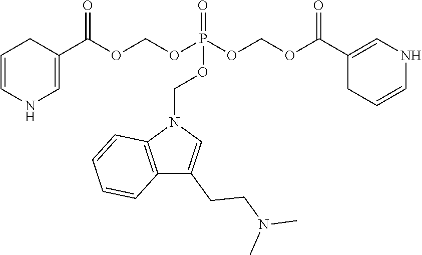 [SMILES: CN(C)CCc1cn(COP(=O)(OCOC(=O)C2=CNC=CC2)OCOC(=O)C2=CNC=CC2)c2ccccc12] |
| | CN(CCC1═CN(C2═C1C═CC═C2)COP(OCOC(C3═CNC═CC3)═O)(OCOC(C4═CNC═CC4)═O)═O)C |
| 246 | 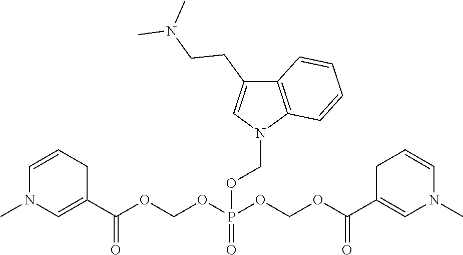 |
| | CN(CCC1═CN(C2═C1C═CC═C2)COP(OCOC(C3═CN(C)C═CC3)═O)(OCOC(C4═CN(C)C═CC4)═O)═O)C |
| 247 | 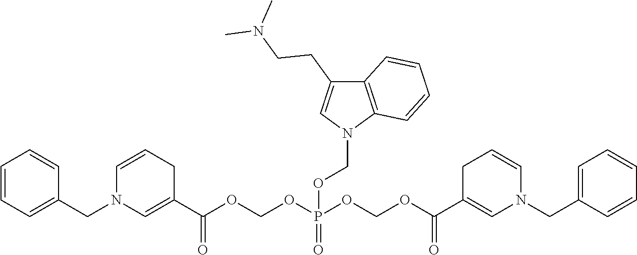 [SMILES: CN(C)CCc1cn(COP(=O)(OCOC(=O)C2=CN(Cc3ccccc3)C=CC2)OCOC(=O)C2=CN(Cc3ccccc3)C=CC2)c2ccccc12] |
| | CN(CCC1═CN(C2═C1C═CC═C2)COP(OCOC(C3═CN(C═CC3)CC4═CC═CC═C4)═O)(OCOC(C5═CN(C═CC5) |
| | CC6═CC═CC═C6)═O)═O)C |
| 248 | 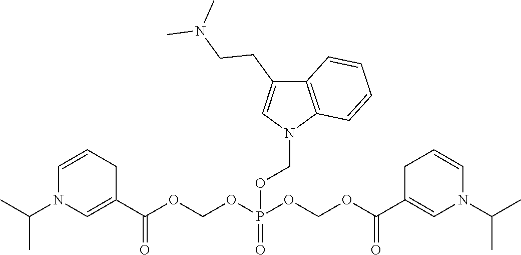 [SMILES: CC(C)N1C=CCC(C(=O)OCOP(=O)(OCOC(=O)C2=CN(C(C)C)C=CC2)OCn2cc(CCN(C)C)c3ccccc32)=C1] |
| | CN(CCC1═CN(C2═C1C═CC═C2)COP(OCOC(C3═CN(C═CC3)C(C)C)═O)(OCOC(C4═CN(C═CC4)C(C) |
| | C)═O)═O)C |
| 249 | 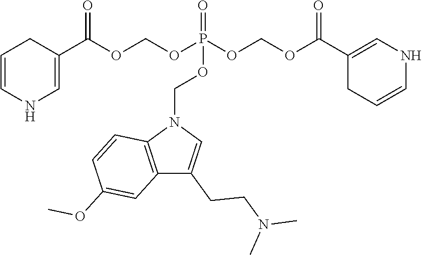 [SMILES: COc1ccc2c(c1)c(CCN(C)C)cn2COP(=O)(OCOC(=O)C1=CNC=CC1)OCOC(=O)C1=CNC=CC1] |
| | CN(CCC1═CN(C2═C1C═C(OC)C═C2)COP(OCOC(C3═CNC═CC3)═O)(OCOC(C4═CNC═CC4)═O)═O)C |
| 250 | 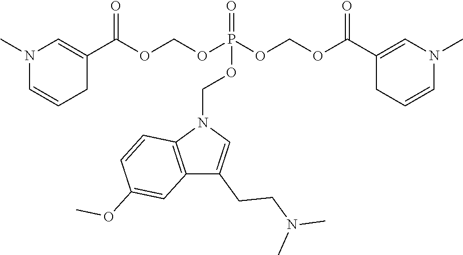 |
| | CN(CCC1═CN(C2═C1C═C(OC)C═C2)COP(OCOC(C3═CN(C)C═CC3)═O)(OCOC(C4═CN(C)C═CC4)═O)═O)C |
| 251 | 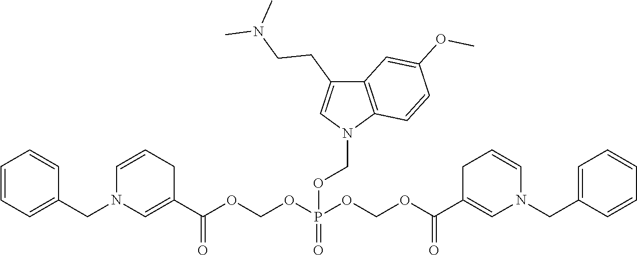 [SMILES: COc1ccc2c(c1)c(CCN(C)C)cn2COP(=O)(OCOC(=O)C1=CN(Cc2ccccc2)C=CC1)OCOC(=O)C1=CN(Cc2ccccc2)C=CC1] |
| | CN(CCC1═CN(C2═C1C═C(OC)C═C2)COP(OCOC(C3═CN(C═CC3)CC4═CC═CC═C4)═O)(OCOC(C5═CN |
| | (C═CC5)CC6═CC═CC═C6)═O)═O)C |
| 252 | 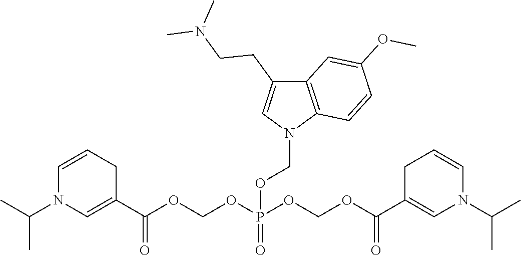 [SMILES: COc1ccc2c(c1)c(CCN(C)C)cn2COP(=O)(OCOC(=O)C1=CN(C(C)C)C=CC1)OCOC(=O)C1=CN(C(C)C)C=CC1] |
| | CN(CCC1═CN(C2═C1C═C(OC)C═C2)COP(OCOC(C3═CN(C═CC3)C(C)C)═O)(OCOC(C4═CN(C═CC4)C(C) |
| | C)═O)═O)C |
| 253 | 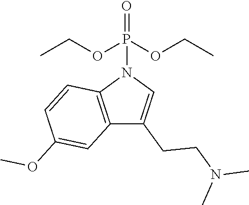 |
| | CN(CCC1═CN(C2═C1C═C(OC)C═C2)P(OCC)(OCC)═O)C |
| 254 | 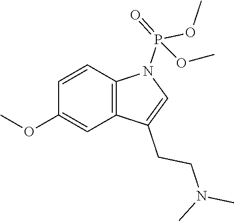 |
| | CN(CCC1═CN(C2═C1C═C(OC)C═C2)P(OC)(OC)═O)C |
| 255 | 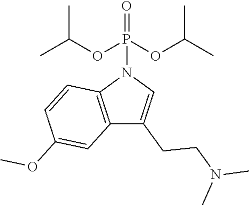 |
| | CN(CCC1═CN(C2═C1C═C(OC)C═C2)P(OC)(C)C)(OC(C)C)═O)C |
| 256 | 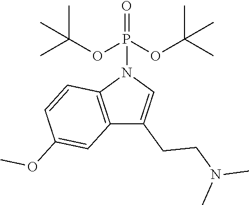 |
| | CN(CCC1═CN(C2═C1C═C(OC)C═C2)P(OC(C)(C)C)(OC(C)(C)C)═O)C |
| 257 | 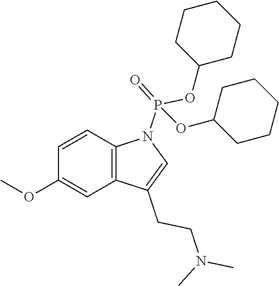 [SMILES: COc1ccc2c(c1)c(CCN(C)C)cn2P(=O)(OC1CCCCC1)OC1CCCCC1] |
| | CN(CCC1═CN(C2═C1C═C(OC)C═C2)P(OC3CCCCC3)(OC4CCCCC4)═O)C |
| 258 | 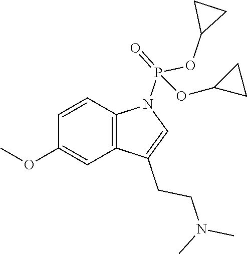 [SMILES: COc1ccc2c(c1)c(CCN(C)C)cn2P(=O)(OC1CC1)OC1CC1] |
| | CN(CCC1═CN(C2═C1C═C(OC)C═C2)P(OC3CC3)(OC4CC4)═O)C |
| 259 | 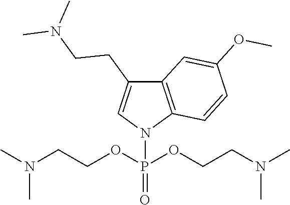 |
| | CN(CCC1═CN(C2═C1C═C(OC)C═C2)P(OCCN(C)C)(OCCN(C)C)═O)C |
| 260 | 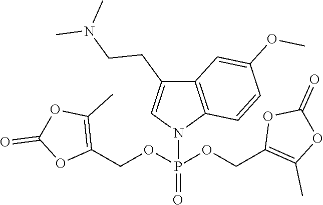 [SMILES: COc1ccc2c(c1)c(CCN(C)C)cn2P(=O)(OCc1oc(=O)oc1C)OCc1oc(=O)oc1C] |
| | CN(CCC1═CN(C2═C1C═C(OC)C═C2)P(OCC3═C(OC(O3)═O)C)(OCC(OC4═O)═C(O4)C)═O)C |
| 261 | 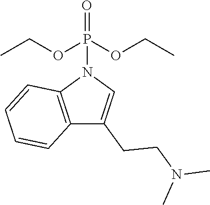 [SMILES: CCOP(=O)(OCC)n1cc(CCN(C)C)c2ccccc21] |
| | CN(CCC1═CN(C2═C1C═CC═C2)P(OCC)(OCC)═O)C |
| 262 | 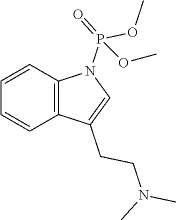 [SMILES: COP(=O)(OC)n1cc(CCN(C)C)c2ccccc21] |
| | CN(CCC1═CN(C2═C1C═CC═C2)P(OC)(OC)═O)C |
| 263 | 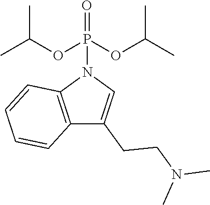 [SMILES: CC(C)OP(=O)(OC(C)C)n1cc(CCN(C)C)c2ccccc21] |
| | CN(CCC1═CN(C2═C1C═CC═C2)P(OC(C)C)(OC(C)C)═O)C |
| 264 | 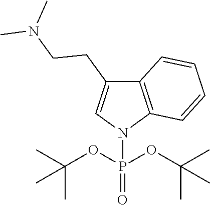 [SMILES: CN(C)CCc1cn(P(=O)(OC(C)(C)C)OC(C)(C)C)c2ccccc12] |
| | CN(CCC1═CN(C2═C1C═CC═C2)P(OC(C)(C)(OC(C)(C)C)═O)C |
| 265 | 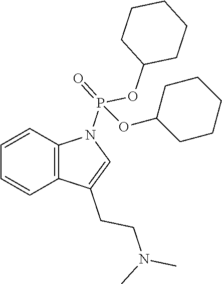 [SMILES: CN(C)CCc1cn(P(=O)(OC2CCCCC2)OC2CCCCC2)c2ccccc12] |
| | CN(CCC1═CN(C2═C1C═CC═C2)P(OC3CCCCC3)(OC4CCCCC4)═O)C |
| 266 | 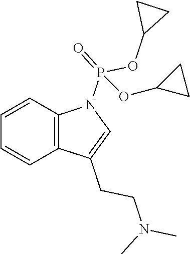 [SMILES: CN(C)CCc1cn(P(=O)(OC2CC2)OC2CC2)c2ccccc12] |
| | CN(CCC1═CN(C2═C1C═CC═C2)P(OC3CC3)(OC4CC4)═O)C |
| 267 | 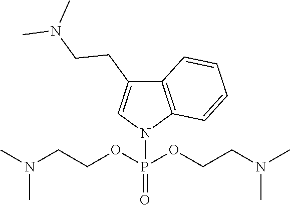 |
| | CN(CCC1═CN(C2═C1C═CC═C2)P(OCCN(C)C)(OCCN(C)C)═O)C |
| 268 | 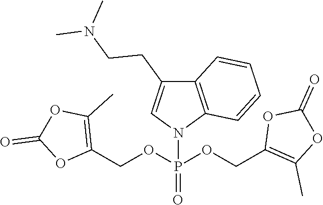 [SMILES: Cc1oc(=O)oc1COP(=O)(OCc1oc(=O)oc1C)n1cc(CCN(C)C)c2ccccc21] |
| | CN(CCC1═CN(C2═C1C═CC═C2)P(OCC3═C(OC(O3)═O)C)(OCC(OC4═O)═C(O4)C)═O)C |
| 269 | 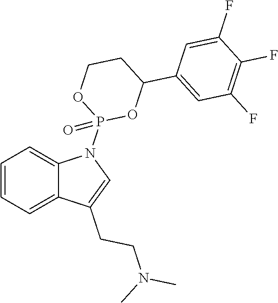 [SMILES: CN(C)CCc1cn(P2(=O)OCCC(c3cc(F)c(F)c(F)c3)O2)c2ccccc12] |
| | CN(CCC1═CN(C2═CC═CC═C21)P3(OCCC(O3)C4═CC(F)═C(C(F)═C4)F)═O)C |
| 270 | 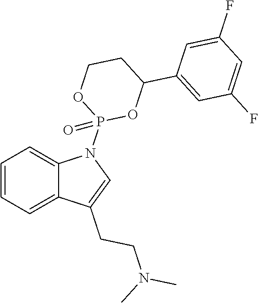 [SMILES: CN(C)CCc1cn(P2(=O)OCCC(c3cc(F)cc(F)c3)O2)c2ccccc12] |
| | CN(CCC1═CN(C2═CC═CC═C21)P3(OCCC(O3)C4═CC(F)═CC(F)═C4)═O)C |
| 271 | 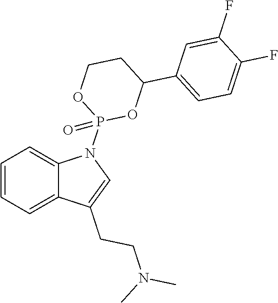 [SMILES: CN(C)CCc1cn(P2(=O)OCCC(c3ccc(F)c(F)c3)O2)c2ccccc12] |
| | CN(CCC1═CN(C2═CC═CC═C21)P3(OCCC(O3)C4═CC═C(C(F)═C4)F)═O)C |
| 272 | 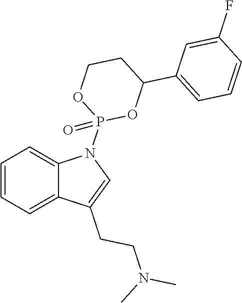 [SMILES: CN(C)CCc1cn(P2(=O)OCCC(c3cccc(F)c3)O2)c2ccccc12] |
| | CN(CCC1═CN(C2═CC═CC═C21)P3(OCCC(O3)C4═CC═CC(F)═C4)═O)C |
| 273 | 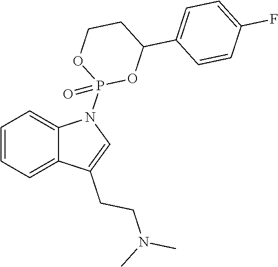 [SMILES: CN(C)CCc1cn(P2(=O)OCCC(c3ccc(F)cc3)O2)c2ccccc12] |
| | CN(CCC1═CN(C2═CC═CC═C21)P3(OCCC(O3)C4═CC═C(C═C4)F)═O)C |
| 274 | 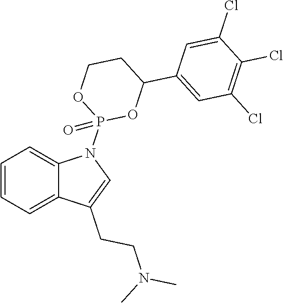 [SMILES: CN(C)CCc1cn(P2(=O)OCCC(c3cc(Cl)c(Cl)c(Cl)c3)O2)c2ccccc12] |
| | CN(CCC1═CN(C2═CC═CC═C21)P3(OCCC(O3)C4═CC(Cl)═C(C(Cl)═C4)Cl)═O)C |
| 275 | 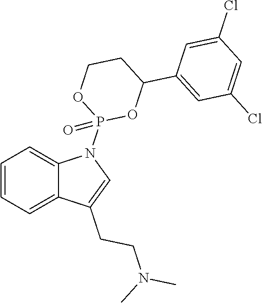 [SMILES: CN(C)CCc1cn(P2(=O)OCCC(c3cc(Cl)cc(Cl)c3)O2)c2ccccc12] |
| | CN(CCC1═CN(C2═CC═CC═C21)P3(OCCC(O3)C4═CC(Cl)═CC(Cl)═C4)═O)C |
| 276 | 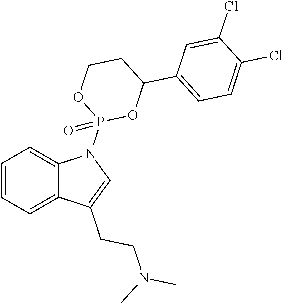 [SMILES: CN(C)CCc1cn(P2(=O)OCCC(c3ccc(Cl)c(Cl)c3)O2)c2ccccc12] |
| | CN(CCC1═CN(C2═CC═CC═C21)P3(OCCC(O3)C4═CC═C(C(Cl)═C4)Cl)═O)C |
| 277 | 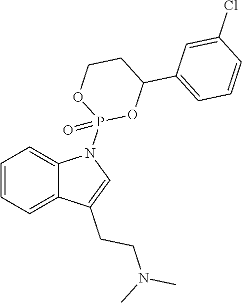 [SMILES: CN(C)CCc1cn(P2(=O)OCCC(c3cccc(Cl)c3)O2)c2ccccc12] |
| | CN(CCC1═CN(C2═CC═CC═C21)P3(OCCC(O3)C4═CC═CC(Cl)═C4)═O)C |
| 278 | 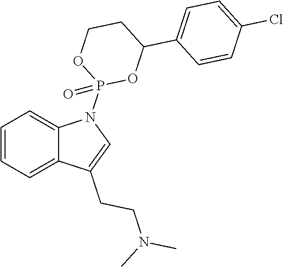 [SMILES: CN(C)CCc1cn(P2(=O)OCCC(c3ccc(Cl)cc3)O2)c2ccccc12] |
| | CN(CCC1═CN(C2═CC═CC═C21)P3(OCCC(O3)C4═CC═C(C═C4)Cl)═O)C |
| 279 | 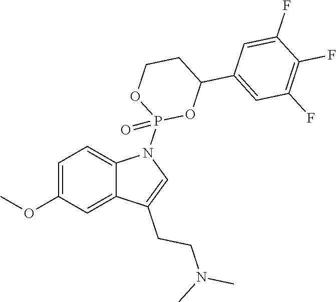 [SMILES: COc1ccc2c(c1)c(CCN(C)C)cn2P1(=O)OCCC(c2cc(F)c(F)c(F)c2)O1] |
| | CN(CCC1═CN(C2═CC═C(OC)C═C21)P3(OCCC(O3)C4═CC(F)═C(C(F)═C4)F)═O)C |
| 280 | 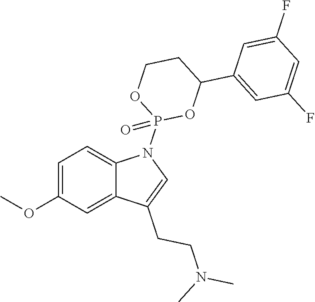 [SMILES: COc1ccc2c(c1)c(CCN(C)C)cn2P1(=O)OCCC(c2cc(F)cc(F)c2)O1] |
| | CN(CCC1═CN(C2═CC═C(OC)C═C21)P3(OCCC(O3)C4═CC(F)═CC(F)═C4)═O)C |
| 281 | 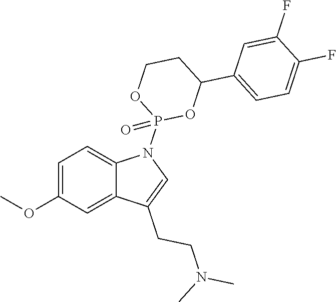 [SMILES: COc1ccc2c(c1)c(CCN(C)C)cn2P1(=O)OCCC(c2ccc(F)c(F)c2)O1] |
| | CN(CCC1═CN(C2═CC═C(OC)C═C21)P3(OCCC(O3)C4═CC═C(C(F)═C4)F)═O)C |
| 282 | 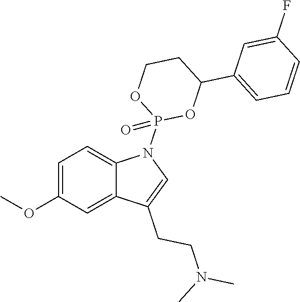 [SMILES: COc1ccc2c(c1)c(CCN(C)C)cn2P1(=O)OCCC(c2cccc(F)c2)O1] |
| | CN(CCC1═CN(C2═CC═C(OC)C═C21)P3(OCCC(O3)C4═CC═CC(F)═C4)═O)C |
| 283 | 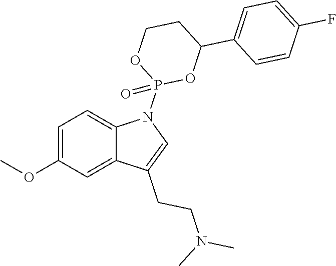 [SMILES: COc1ccc2c(c1)c(CCN(C)C)cn2P1(=O)OCCC(c2ccc(F)cc2)O1] |
| | CN(CCC1═CN(C2═CC═C(OC)C═C21)P3(OCCC(O3)C4═CC═C(C═C4)F)═O)C |
| 284 | 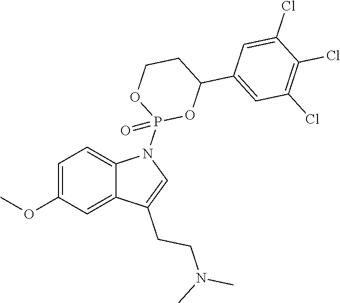 [SMILES: COc1ccc2c(c1)c(CCN(C)C)cn2P1(=O)OCCC(c2cc(Cl)c(Cl)c(Cl)c2)O1] |
| | CN(CCC1═CN(C2═CC═C(OC)C═C21)P3(OCCC(O3)C4═CC(Cl)═C(C(Cl)═C4)Cl)═O)C |
| 285 | 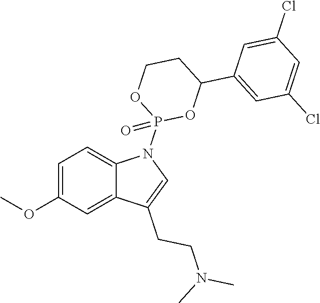 [SMILES: COc1ccc2c(c1)c(CCN(C)C)cn2P1(=O)OCCC(c2cc(Cl)cc(Cl)c2)O1] |
| | CN(CCC1═CN(C2═CC═C(OC)C═C21)P3(OCCC(O3)C4═CC(Cl)═CC(Cl)═C4)═O)C |
| 286 | 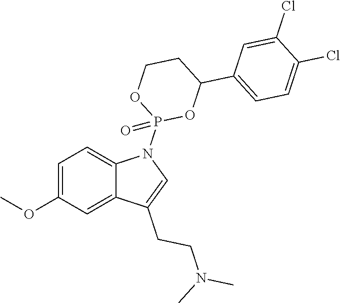 [SMILES: COc1ccc2c(c1)c(CCN(C)C)cn2P1(=O)OCCC(c2ccc(Cl)c(Cl)c2)O1] |
| | CN(CCC1═CN(C2═CC═C(OC)C═C21)P3(OCCC(O3)C4═CC═C(C(Cl)═C4)Cl)═O)C |
| 287 | 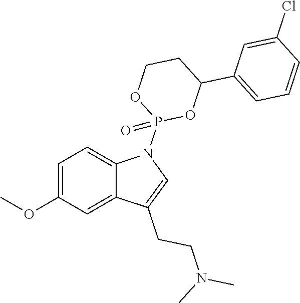 [SMILES: COc1ccc2c(c1)c(CCN(C)C)cn2P1(=O)OCCC(c2cccc(Cl)c2)O1] |
| | CN(CCC1═CN(C2═CC═C(OC)C═C21)P3(OCCC(O3)C4═CC═CC(Cl)═C4)═O)C |
| 288 | 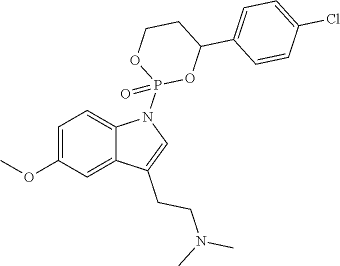 [SMILES: COc1ccc2c(c1)c(CCN(C)C)cn2P1(=O)OCCC(c2ccc(Cl)cc2)O1] |
| | CN(CCC1═CN(C2═CC═C(OC)C═C21)P3(OCCC(O3)C4═CC═C(C═C4)Cl)═O)C |
| 289 | 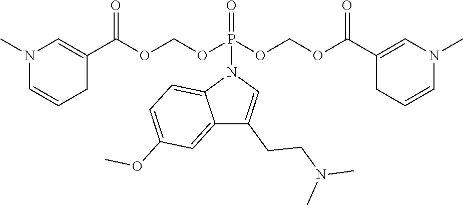 |
| | CN(CCC1═CN(C2═CC═C(OC)C═C21)P(OCOC(C3═CN(C)C═CC3)═O)(OCOC(C4═CN(C)C═CC4)═O)═O)C |
| 290 | 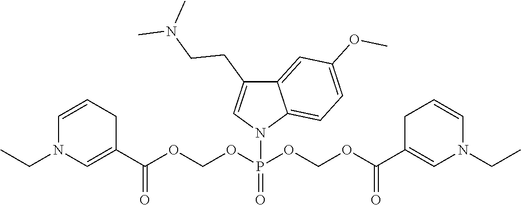 [SMILES: CCN1C=CCC(C(=O)OCOP(=O)(OCOC(=O)C2=CN(CC)C=CC2)n2cc(CCN(C)C)c3cc(OC)ccc32)=C1] |
| | CN(CCC1═CN(C2═CC═C(OC)C═C21)P(OCOC(C3═CN(C═CC3)CC)═O)(OCOC(C4═CN(C═CC4)CC)═O)═O)C |
| 291 | 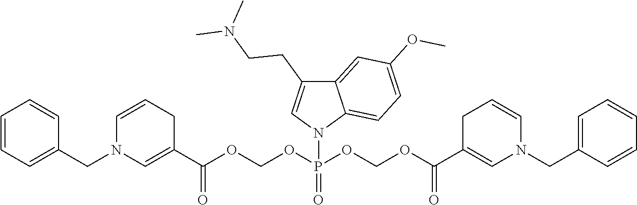 [SMILES: COc1ccc2c(c1)c(CCN(C)C)cn2P(=O)(OCOC(=O)C1=CN(Cc2ccccc2)C=CC1)OCOC(=O)C1=CN(Cc2ccccc2)C=CC1] |
| | CN(CCC1═CN(C2═CC═C(OC)C═C21)P(OCOC(C3═CN(C═CC3)CC4═CC═CC═C4)═O)(OCOC(C5═CN(C═CC5) |
| | CC6═CC═CC═C6)═O)═O)C |
| 292 | 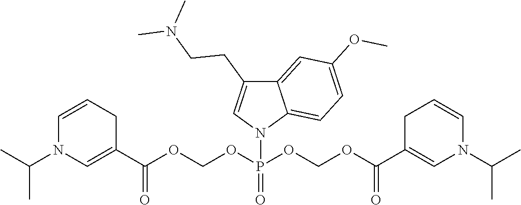 [SMILES: COc1ccc2c(c1)c(CCN(C)C)cn2P(=O)(OCOC(=O)C1=CN(C(C)C)C=CC1)OCOC(=O)C1=CN(C(C)C)C=CC1] |
| | CN(CCC1═CN(C2═CC═C(OC)C═C21)P(OCOC(C3═CN(C═CC3)C(C)C)═O)(OCOC(C4═CN(C═CC4)C(C) |
| | C)═O)═O)C |
| 293 | 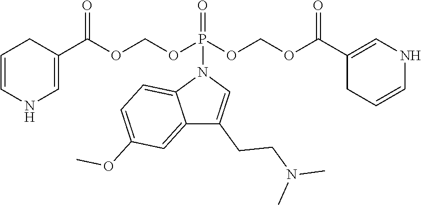 [SMILES: COc1ccc2c(c1)c(CCN(C)C)cn2P(=O)(OCOC(=O)C1=CNC=CC1)OCOC(=O)C1=CNC=CC1] |
| | CN(CCC1═CN(C2═CC═C(OC)C═C21)P(OCOC(C3═CNC═CC3)═O)(OCOC(C4═CNC═CC4)═O)═O)C |
| 294 | 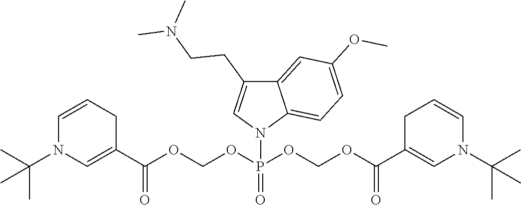 [SMILES: COc1ccc2c(c1)c(CCN(C)C)cn2P(=O)(OCOC(=O)C1=CN(C(C)(C)C)C=CC1)OCOC(=O)C1=CN(C(C)(C)C)C=CC1] |
| | CN(CCC1═CN(C2═CC═C(OC)C═C21)P(OCOC(C3═CN(C═CC3)C(C)(C)C)═O)(OCOC(C4═CN(C═CC4)C(C)(C) |
| | C)═O)═O)C |
| 295 | 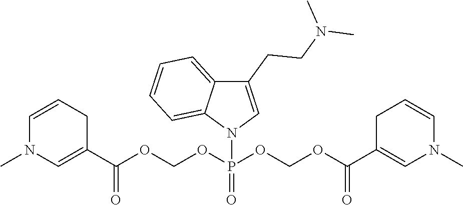 |
| | CN(CCC1═CN(C2═CC═CC═C21)P(OCOC(C3═CN(C)C═CC3)═O)(OCOC(C4═CN(C)C═CC4)═O)═O)C |
| 296 | 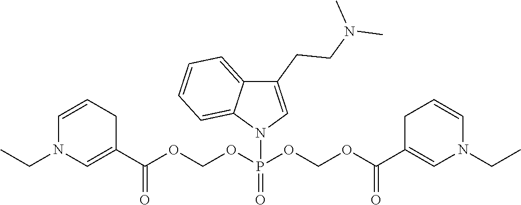 [SMILES: CCN1C=CCC(C(=O)OCOP(=O)(OCOC(=O)C2=CN(CC)C=CC2)n2cc(CCN(C)C)c3ccccc32)=C1] |
| | CN(CCC1═CN(C2═CC═CC═C21)P(OCOC(C3═CN(C═CC3)CC)═O)(OCOC(C4═CN(C═CC4)CC)═O)═O)C |
| 297 | 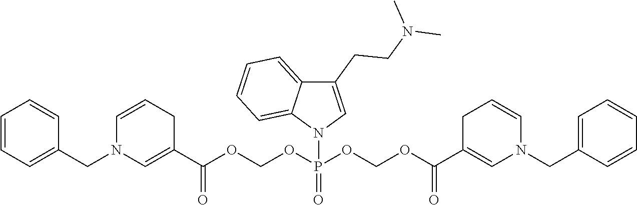 [SMILES: CN(C)CCc1cn(P(=O)(OCOC(=O)C2=CN(Cc3ccccc3)C=CC2)OCOC(=O)C2=CN(Cc3ccccc3)C=CC2)c2ccccc12] |
| | CN(CCC1═CN(C2═CC═CC═C21)P(OCOC(C3═CN(C═CC3)CC4═CC═CC═C4)═O)(OCOC(C5═CN(C═CC5) |
| | CC6═CC═CC═C6)═O)═O)C |
| 298 | 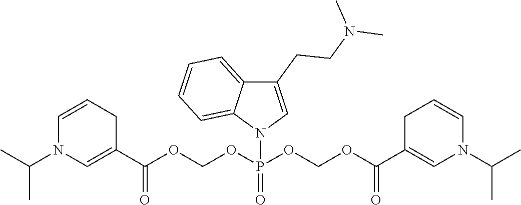 [SMILES: CC(C)N1C=CCC(C(=O)OCOP(=O)(OCOC(=O)C2=CN(C(C)C)C=CC2)n2cc(CCN(C)C)c3ccccc32)=C1] |
| | CN(CCC1═CN(C2═CC═CC═C21)P(OCOC(C3═CN(C═CC3)C(C)C)═O)(OCOC(C4═CN(C═CC4)C(C)C)═O)═O)C |
| 299 | 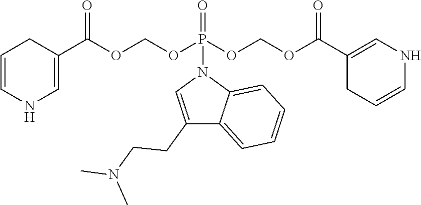 [SMILES: CN(C)CCc1cn(P(=O)(OCOC(=O)C2=CNC=CC2)OCOC(=O)C2=CNC=CC2)c2ccccc12] |
| | CN(CCC1═CN(C2═CC═CC═C21)P(OCOC(C3═CNC═CC3)═O)(OCOC(C4═CNC═CC4)═O)═O)C |
| 300 | 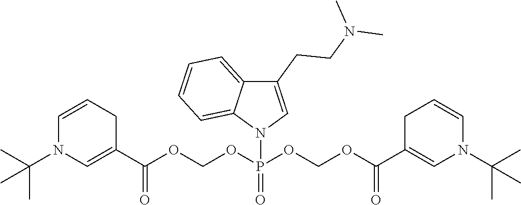 [SMILES: CN(C)CCc1cn(P(=O)(OCOC(=O)C2=CN(C(C)(C)C)C=CC2)OCOC(=O)C2=CN(C(C)(C)C)C=CC2)c2ccccc12] |
| | CN(CCC1═CN(C2═CC═CC═C21)P(OCOC(C3═CN(C═CC3)C(C)(C)C)═O)(OCOC(C4═CN(C═CC4)C(C)(C) |
| | C)═O)═O)C |
| 301 | 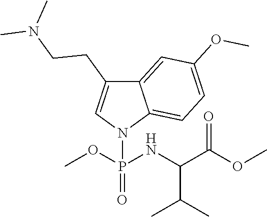 |
| | CN(CCC1═CN(C2═C1C═C(OC)C═C2)P(OC)(NC(C(OC)═O)C(C)C)═O)C |
| 302 | 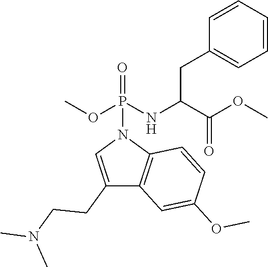 [SMILES: COC(=O)C(Cc1ccccc1)NP(=O)(OC)n1cc(CCN(C)C)c2cc(OC)ccc21] |
| | CN(CCC1═CN(C2═C1C═C(OC)C═C2)P(OC)(NC(C(OC)═O)CC3═CC═CC═C3)═O)C |
| 303 | 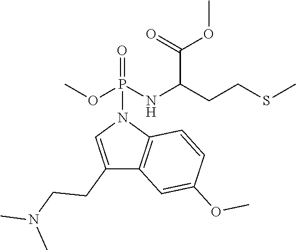 |
| | CN(CCC1═CN(C2═C1C═C(OC)C═C2)P(OC)(NC(C(OC)═O)CCSC)═O)C |
| 304 | 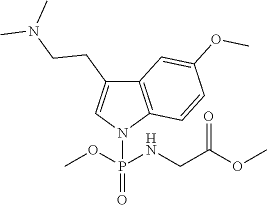 |
| | CN(CCC1═CN(C2═C1C═C(OC)C═C2)P(OC)(NCC(OC)═O)═O)C |
| 305 | 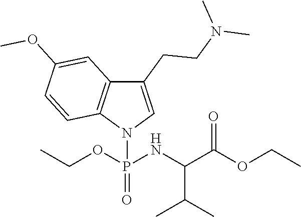 [SMILES: CCOC(=O)C(NP(=O)(OCC)n1cc(CCN(C)C)c2cc(OC)ccc21)C(C)C] |
| | CN(CCC1═CN(C2═C1C═C(OC)C═C2)P(OCC)(NC(C(OCC)═O)C(C)C)═O)C |
| 306 | 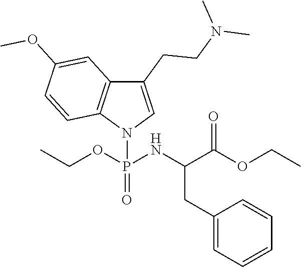 [SMILES: CCOC(=O)C(Cc1ccccc1)NP(=O)(OCC)n1cc(CCN(C)C)c2cc(OC)ccc21] |
| | CN(CCC1═CN(C2═C1C═C(OC)C═C2)P(OCC)(NC(C(OCC)═O)CC3═CC═CC═C3)═O)C |
| 307 | 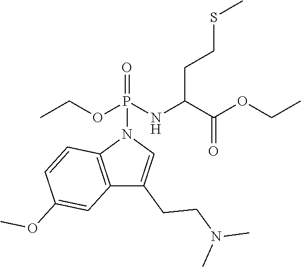 [SMILES: CCOC(=O)C(CCSC)NP(=O)(OCC)n1cc(CCN(C)C)c2cc(OC)ccc21] |
| | CN(CCC1═CN(C2═C1C═C(OC)C═C2)P(OCC)(NC(C(OCC)═O)CCSC)═O)C |
| 308 | 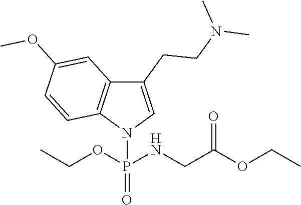 [SMILES: CCOC(=O)CNP(=O)(OCC)n1cc(CCN(C)C)c2cc(OC)ccc21] |
| | CN(CCC1═CN(C2═C1C═C(OC)C═C2)P(OCC)(NCC(OCC)═O)═O)C |
| 309 | 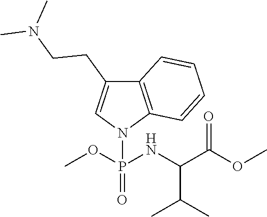 |
| | CN(CCC1═CN(C2═C1C═CC═C2)P(OC)(NC(C(OC)═O)C(C)C)═O)C |
| 310 | 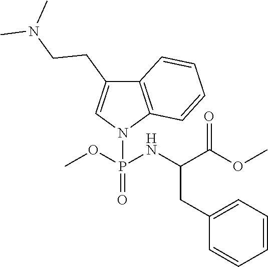 [SMILES: COC(=O)C(Cc1ccccc1)NP(=O)(OC)n1cc(CCN(C)C)c2ccccc21] |
| | CN(CCC1═CN(C2═C1C═CC═C2)P(OC)(NC(C(OC)═O)CC3═CC═CC═C3)═O)C |
| 311 | 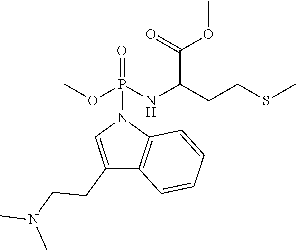 |
| | CN(CCC1═CN(C2═C1C═CC═C2)P(OC)(NC(C(OC)═O)CCSC)═O)C |
| 312 | 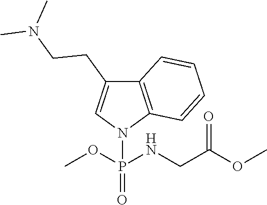 |
| | CN(CCC1═CN(C2═C1C═CC═C2)P(OC)(NCC(OC)═O)═O)C |
| 313 | 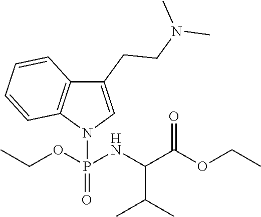 [SMILES: CCOC(=O)C(NP(=O)(OCC)n1cc(CCN(C)C)c2ccccc21)C(C)C] |
| | CN(CCC1═CN(C2═C1C═CC═C2)P(OCC)(NC(C(OCC)═O)C(C)C)═O)C |
| 314 | 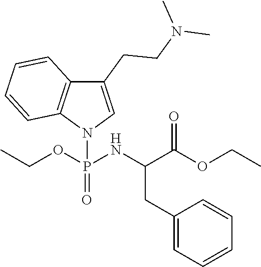 [SMILES: CCOC(=O)C(Cc1ccccc1)NP(=O)(OCC)n1cc(CCN(C)C)c2ccccc21] |
| | CN(CCC1═CN(C2═C1C═CC═C2)P(OCC)(NC(C(OCC)═O)CC3═CC═CC═C3)═O)C |
| 315 | 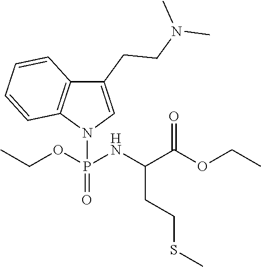 [SMILES: CCOC(=O)C(CCSC)NP(=O)(OCC)n1cc(CCN(C)C)c2ccccc21] |
| | CN(CCC1═CN(C2═C1C═CC═C2)P(OCC)(NC(C(OCC)═O)CCSC)═O)C |
| 316 | 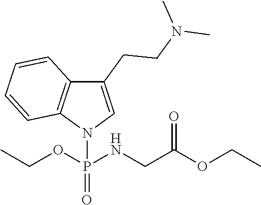 [SMILES: CCOC(=O)CNP(=O)(OCC)n1cc(CCN(C)C)c2ccccc21] |
| | CN(CCC1═CN(C2═C1C═CC═C2)P(OCC)(NCC(OCC)═O)═O)C |
| 317 | 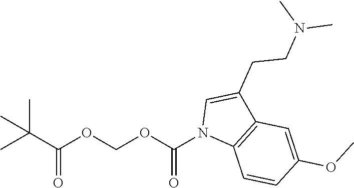 [SMILES: COc1ccc2c(c1)c(CCN(C)C)cn2C(=O)OCOC(=O)C(C)(C)C] |
| | CN(CCC1═CN(C2═C1C═C(OC)C═C2)C(OCOC(C(C)(C)C)═O)═O)C |
| 318 | 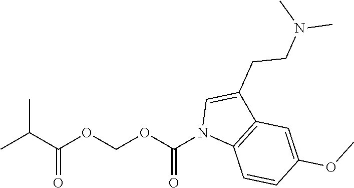 [SMILES: COc1ccc2c(c1)c(CCN(C)C)cn2C(=O)OCOC(=O)C(C)C] |
| | CN(CCC1═CN(C2═C1C═C(OC)C═C2)C(OCOC(C(C)C)═O)═O)C |
| 319 | 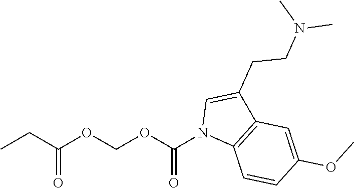 [SMILES: CCC(=O)OCOC(=O)n1cc(CCN(C)C)c2cc(OC)ccc21] |
| | CN(CCC1═CN(C2═C1C═C(OC)C═C2)C(OCOC(CC)═O)═O)C |
| 320 | 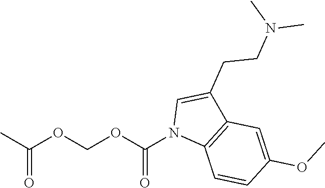 [SMILES: COc1ccc2c(c1)c(CCN(C)C)cn2C(=O)OCOC(C)=O] |
| | CN(CCC1═CN(C2═C1C═C(OC)C═C2)C(OCOC(C)═O)═O)C |
| 321 | 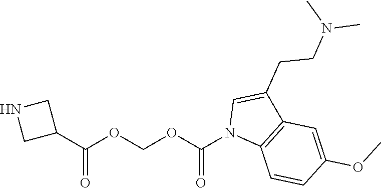 [SMILES: COc1ccc2c(c1)c(CCN(C)C)cn2C(=O)OCOC(=O)C1CNC1] |
| | CN(CCC1═CN(C2═C1C═C(OC)C═C2)C(OCOC(C3CNC3)═O)═O)C |
| 322 | 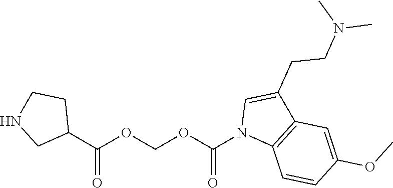 [SMILES: COc1ccc2c(c1)c(CCN(C)C)cn2C(=O)OCOC(=O)C1CCNC1] |
| | CN(CCC1═CN(C2═C1C═C(OC)C═C2)C(OCOC(C3CNCC3)═O)═O)C |
| 323 | 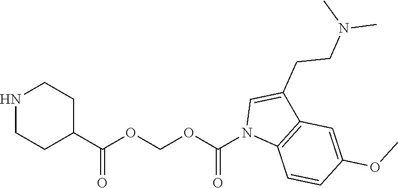 [SMILES: COc1ccc2c(c1)c(CCN(C)C)cn2C(=O)OCOC(=O)C1CCNCC1] |
| | CN(CCC1═CN(C2═C1C═C(OC)C═C2)C(OCOC(C3CCNCC3)═O)═O)C |
| 324 | 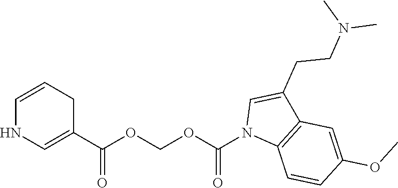 [SMILES: COc1ccc2c(c1)c(CCN(C)C)cn2C(=O)OCOC(=O)C1=CNC=CC1] |
| | CN(CCC1═CN(C2═C1C═C(OC)C═C2)C(OCOC(C3═CNC═CC3)═O)═O)C |
| 325 | 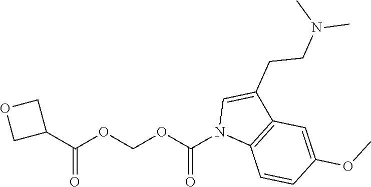 [SMILES: COc1ccc2c(c1)c(CCN(C)C)cn2C(=O)OCOC(=O)C1COC1] |
| | CN(CCC1═CN(C2═C1C═C(OC)C═C2)C(OCOC(C3COC3)═O)═O)C |
| 326 | 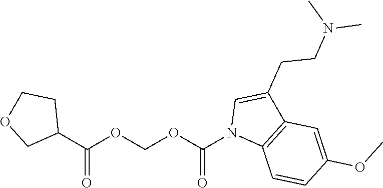 [SMILES: COc1ccc2c(c1)c(CCN(C)C)cn2C(=O)OCOC(=O)C1CCOC1] |
| | CN(CCC1═CN(C2═C1C═C(OC)C═C2)C(OCOC(C3COCC3)═O)═O)C |
| 327 | 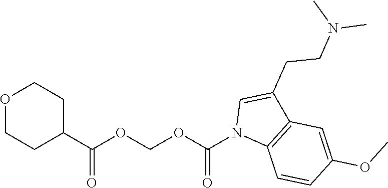 [SMILES: COc1ccc2c(c1)c(CCN(C)C)cn2C(=O)OCOC(=O)C1CCOCC1] |
| | CN(CCC1═CN(C2═C1C═C(OC)C═C2)C(OCOC(C3CCOCC3)═O)═O)C |
| 328 | 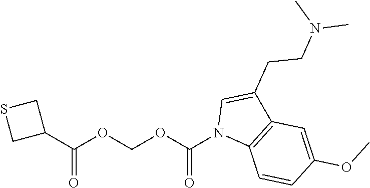 [SMILES: COc1ccc2c(c1)c(CCN(C)C)cn2C(=O)OCOC(=O)C1CSC1] |
| | CN(CCC1═CN(C2═C1C═C(OC)C═C2)C(OCOC(C3CSC3)═O)═O)C |
| 329 | 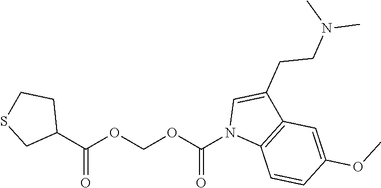 [SMILES: COc1ccc2c(c1)c(CCN(C)C)cn2C(=O)OCOC(=O)C1CCSC1] |
| | CN(CCC1═CN(C2═C1C═C(OC)C═C2)C(OCOC(C3CSCC3)═O)═O)C |
| 330 | 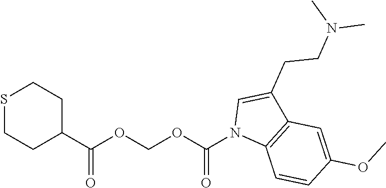 [SMILES: COc1ccc2c(c1)c(CCN(C)C)cn2C(=O)OCOC(=O)C1CCSCC1] |
| | CN(CCC1═CN(C2═C1C═C(OC)C═C2)C(OCOC(C3CCSCC3)═O)═O)C |
| 331 | 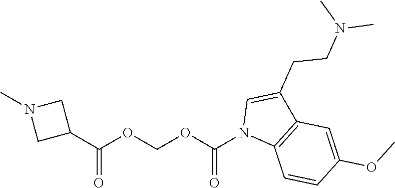 |
| | CN(CCC1═CN(C2═C1C═C(OC)C═C2)C(OCOC(C3CN(C)C3)═O)═O)C |
| 332 | 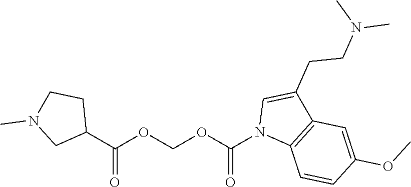 |
| | CN(CCC1═CN(C2═C1C═C(OC)C═C2)C(OCOC(C3CN(C)CC3)═O)═O)C |
| 333 | 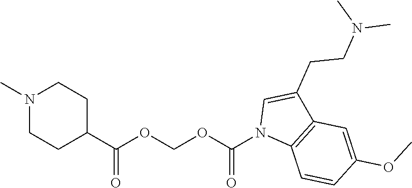 |
| | CN(CCC1═CN(C2═C1C═C(OC)C═C2)C(OCOC(C3CCN(C)CC3)═O)═O)C |
| 334 | 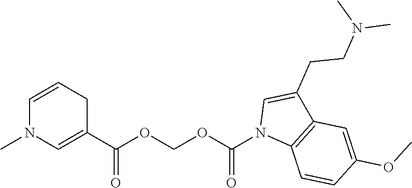 |
| | CN(CCC1═CN(C2═C1C═C(OC)C═C2)C(OCOC(C3═CN(C)C═CC3)═O)═O)C |
| 335 | 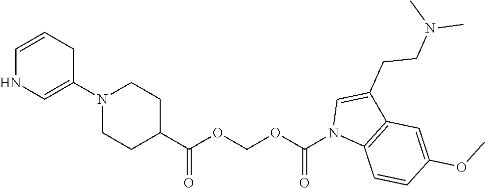 [SMILES: COc1ccc2c(c1)c(CCN(C)C)cn2C(=O)OCOC(=O)C1CCN(C2=CNC=CC2)CC1] |
| | CN(CCC1═CN(C2═C1C═C(OC)C═C2)C(OCOC(C3CCN(CC3)C4═CNC═CC4)═O)═O)C |
| 336 | 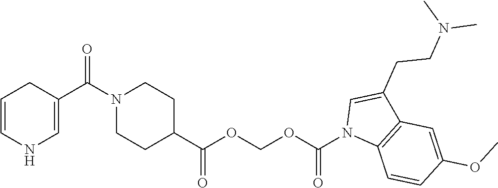 [SMILES: COc1ccc2c(c1)c(CCN(C)C)cn2C(=O)OCOC(=O)C1CCN(C(=O)C2=CNC=CC2)CC1] |
| | CN(CCC1═CN(C2═C1C═C(OC)C═C2)C(OCOC(C3CCN(CC3)C(C4═CNC═CC4)═O)═O)═O)C |
| 337 | 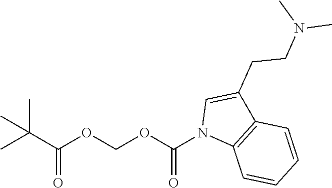 [SMILES: CN(C)CCc1cn(C(=O)OCOC(=O)C(C)(C)C)c2ccccc12] |
| | CN(CCC1═CN(C2═C1C═CC═C2)C(OCOC(C(C)(C)C)═O)═O)C |
| 338 | 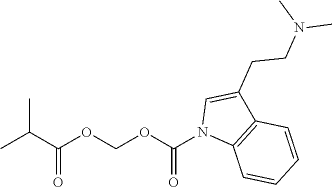 [SMILES: CC(C)C(=O)OCOC(=O)n1cc(CCN(C)C)c2ccccc21] |
| | CN(CCC1═CN(C2═C1C═CC═C2)C(OCOC(C(C)C)═O)═O)C |
| 339 | 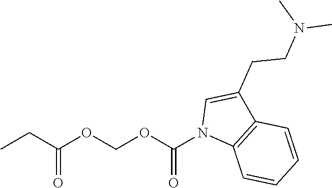 [SMILES: CCC(=O)OCOC(=O)n1cc(CCN(C)C)c2ccccc21] |
| | CN(CCC1═CN(C2═C1C═CC═C2)C(OCOC(CC)═O)═O)C |
| 340 | 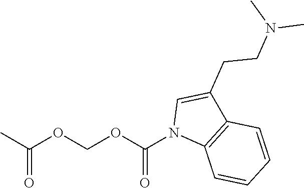 [SMILES: CC(=O)OCOC(=O)n1cc(CCN(C)C)c2ccccc21] |
| | CN(CCC1═CN(C2═C1C═CC═C2)C(OCOC(C)═O)═O)C |
| 341 | 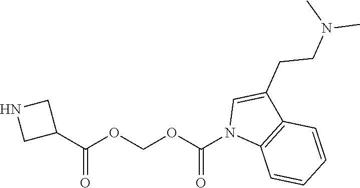 [SMILES: CN(C)CCc1cn(C(=O)OCOC(=O)C2CNC2)c2ccccc12] |
| | CN(CCC1═CN(C2═C1C═CC═C2)C(OCOC(C3CNC3)═O)═O)C |
| 342 | 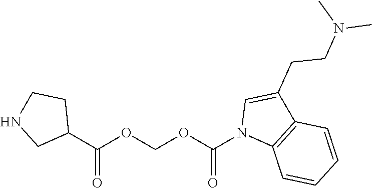 [SMILES: CN(C)CCc1cn(C(=O)OCOC(=O)C2CCNC2)c2ccccc12] |
| | CN(CCC1═CN(C2═C1C═CC═C2)C(OCOC(C3CNCC3)═O)═O)C |
| 343 | 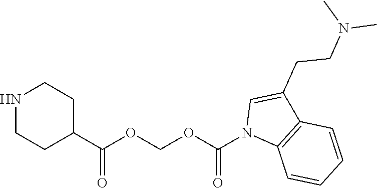 [SMILES: CN(C)CCc1cn(C(=O)OCOC(=O)C2CCNCC2)c2ccccc12] |
| | CN(CCC1═CN(C2═C1C═CC═C2)C(OCOC(C3CCNCC3)═O)═O)C |
| 344 | 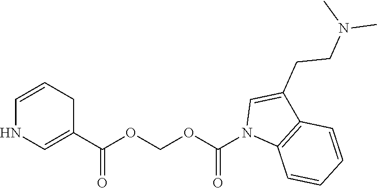 [SMILES: CN(C)CCc1cn(C(=O)OCOC(=O)C2=CNC=CC2)c2ccccc12] |
| | CN(CCC1═CN(C2═C1C═CC═C2)C(OCOC(C3═CNC═CC3)═O)═O)C |
| 345 | 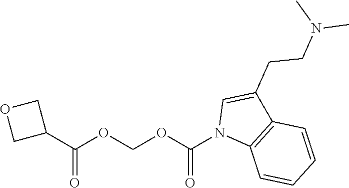 [SMILES: CN(C)CCc1cn(C(=O)OCOC(=O)C2COC2)c2ccccc12] |
| | CN(CCC1═CN(C2═C1C═CC═C2)C(OCOC(C3COC3)═O)═O)C |
| 346 | 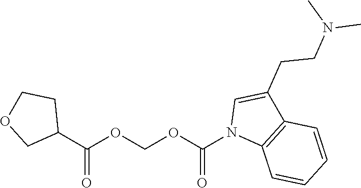 [SMILES: CN(C)CCc1cn(C(=O)OCOC(=O)C2CCOC2)c2ccccc12] |
| | CN(CCC1═CN(C2═C1C═CC═C2)C(OCOC(C3COCC3)═O)═O)C |
| 347 | 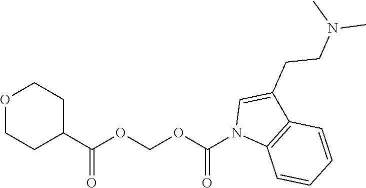 [SMILES: CN(C)CCc1cn(C(=O)OCOC(=O)C2CCOCC2)c2ccccc12] |
| | CN(CCC1═CN(C2═C1C═CC═C2)C(OCOC(C3CCOCC3)═O)═O)C |
| 348 | 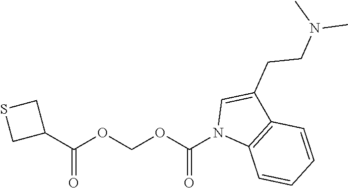 [SMILES: CN(C)CCc1cn(C(=O)OCOC(=O)C2CSC2)c2ccccc12] |
| | CN(CCC1═CN(C2═C1C═CC═C2)C(OCOC(C3CSC3)═O)═O)C |
| 349 | 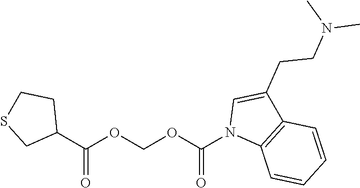 [SMILES: CN(C)CCc1cn(C(=O)OCOC(=O)C2CCSC2)c2ccccc12] |
| | CN(CCC1═CN(C2═C1C═CC═C2)C(OCOC(C3CSCC3)═O)═O)C |
| 350 | 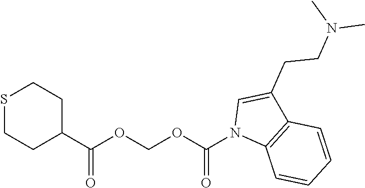 [SMILES: CN(C)CCc1cn(C(=O)OCOC(=O)C2CCSCC2)c2ccccc12] |
| | CN(CCC1═CN(C2═C1C═CC═C2)C(OCOC(C3CCSCC3)═O)═O)C |
| 351 | 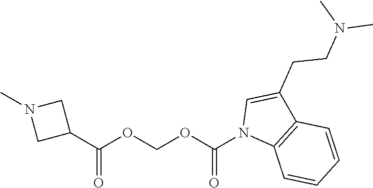 |
| | CN(CCC1═CN(C2═C1C═CC═C2)C(OCOC(C3CN(C)C3)═O)═O)C |
| 352 | 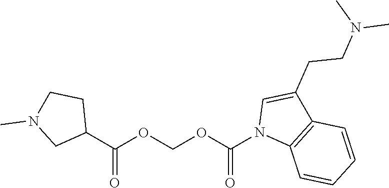 |
| | CN(CCC1═CN(C2═C1C═CC═C2)C(OCOC(C3CN(C)CC3)═O)═O)C |
| 353 | 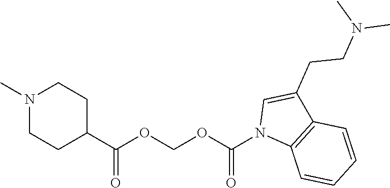 |
| | CN(CCC1═CN(C2═C1C═CC═C2)C(OCOC(C3CCN(C)CC3)═O)═O)C |
| 354 | 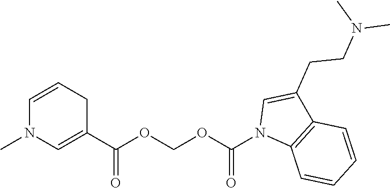 |
| | CN(CCC1═CN(C2═C1C═CC═C2)C(OCOC(C3═CN(C)C═CC3)═O)═O)C |
| 355 | 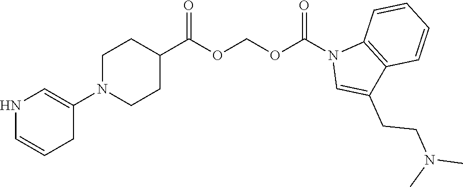 [SMILES: CN(C)CCc1cn(C(=O)OCOC(=O)C2CCN(C3=CNC=CC3)CC2)c2ccccc12] |
| | CN(CCC1═CN(C2═C1C═CC═C2)C(OCOC(C3CCN(CC3)C4═CNC═CC4)═O)═O)C |
| 356 | 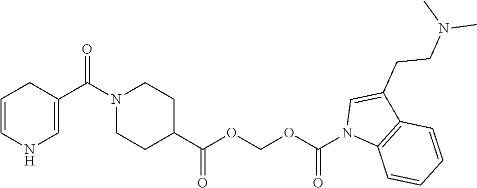 [SMILES: CN(C)CCc1cn(C(=O)OCOC(=O)C2CCN(C(=O)C3=CNC=CC3)CC2)c2ccccc12] |
| | CN(CCC1═CN(C2═C1C═CC═C2)C(OCOC(C3CCN(CC3)C(C4═CNC═CC4)═O)═O)═O)C |
| 357 | 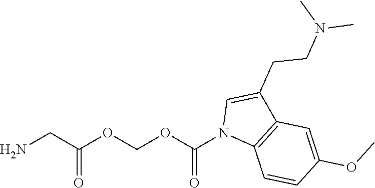 [SMILES: COc1ccc2c(c1)c(CCN(C)C)cn2C(=O)OCOC(=O)CN] |
| | CN(CCC1═CN(C2═C1C═C(OC)C═C2)C(OCOC(CN)═O)═O)C |
| 358 | 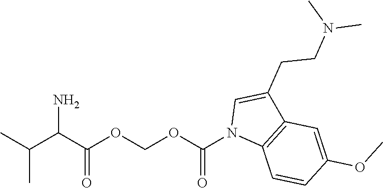 [SMILES: COc1ccc2c(c1)c(CCN(C)C)cn2C(=O)OCOC(=O)C(N)C(C)C] |
| | CN(CCC1═CN(C2═C1C═C(OC)C═C2)C(OCOC(C(C(C)C)N)═O)═O)C |
| 359 | 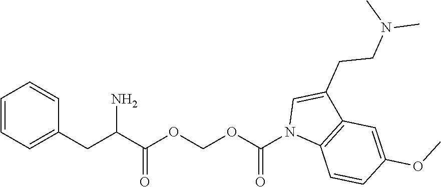 [SMILES: COc1ccc2c(c1)c(CCN(C)C)cn2C(=O)OCOC(=O)C(N)Cc1ccccc1] |
| | CN(CCC1═CN(C2═C1C═C(OC)C═C2)C(OCOC(C(CC3═CC═CC═C3)N)═O)═O)C |
| 360 | 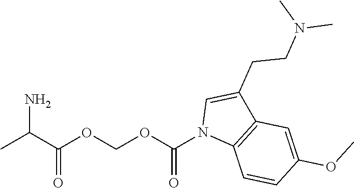 [SMILES: COc1ccc2c(c1)c(CCN(C)C)cn2C(=O)OCOC(=O)C(C)N] |
| | CN(CCC1═CN(C2═C1C═C(OC)C═C2)C(OCOC(C(C)N)═O)═O)C |
| 361 | 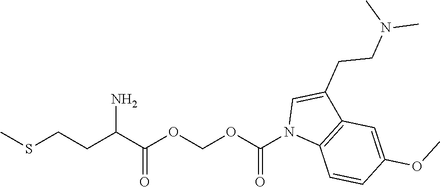 |
| | CN(CCC1═CN(C2═C1C═C(OC)C═C2)C(OCOC(C(CCSC)N)═O)═O)C |
| 362 | 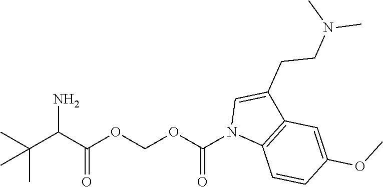 [SMILES: COc1ccc2c(c1)c(CCN(C)C)cn2C(=O)OCOC(=O)C(N)C(C)(C)C] |
| | CN(CCC1═CN(C2═C1C═C(OC)C═C2)C(OCOC(C(C(C)(C)C)N)═O)═O)C |
| 363 | 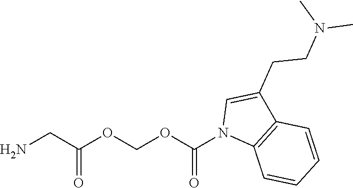 [SMILES: CN(C)CCc1cn(C(=O)OCOC(=O)CN)c2ccccc12] |
| | CN(CCC1═CN(C2═C1C═CC═C2)C(OCOC(CN)═O)═O)C |
| 364 | 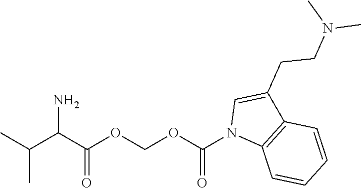 [SMILES: CC(C)C(N)C(=O)OCOC(=O)n1cc(CCN(C)C)c2ccccc21] |
| | CN(CCC1═CN(C2═C1C═CC═C2)C(OCOC(C(C(C)C)N)═O)═O)C |
| 365 | 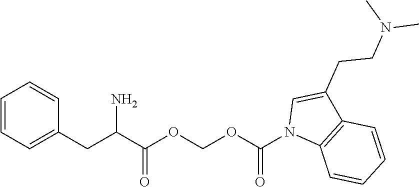 [SMILES: CN(C)CCc1cn(C(=O)OCOC(=O)C(N)Cc2ccccc2)c2ccccc12] |
| | CN(CCC1═CN(C2═C1C═CC═C2)C(OCOC(C(CC3═CC═CC═C3)N)═O)═O)C |
| 366 | 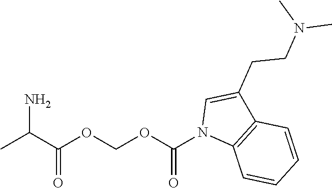 [SMILES: CC(N)C(=O)OCOC(=O)n1cc(CCN(C)C)c2ccccc21] |
| | CN(CCC1═CN(C2═C1C═CC═C2)C(OCOC(C(C)N)═O)═O)C |
| 367 | 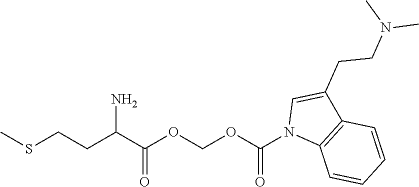 |
| | CN(CCC1═CN(C2═C1C═CC═C2)C(OCOC(C(CCSC)N)═O)═O)C |
| 368 | 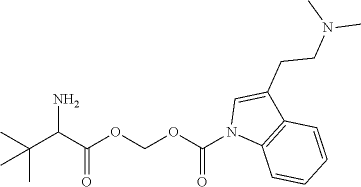 [SMILES: CN(C)CCc1cn(C(=O)OCOC(=O)C(N)C(C)(C)C)c2ccccc12] |
| | CN(CCC1═CN(C2═C1C═CC═C2)C(OCOC(C(C(C)(C)C)N)═O)═O)C |
| 369 | 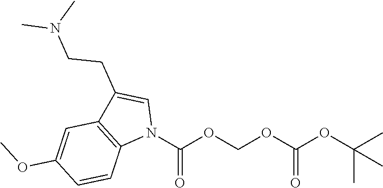 [SMILES: COc1ccc2c(c1)c(CCN(C)C)cn2C(=O)OCOC(=O)OC(C)(C)C] |
| | CN(CCC1═CN(C2═C1C═C(OC)C═C2)C(OCOC(OC(C)(C)C)═O)═O)C |
| 370 | 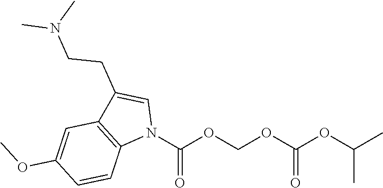 [SMILES: COc1ccc2c(c1)c(CCN(C)C)cn2C(=O)OCOC(=O)OC(C)C] |
| | CN(CCC1═CN(C2═C1C═C(OC)C═C2)C(OCOC(OC(C)C)═O)═O)C |
| 371 | 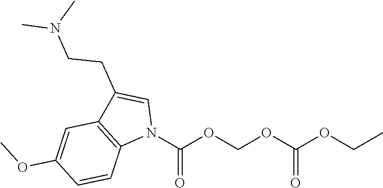 [SMILES: CCOC(=O)OCOC(=O)n1cc(CCN(C)C)c2cc(OC)ccc21] |
| | CN(CCC1═CN(C2═C1C═C(OC)C═C2)C(OCOC(OCC)═O)═O)C |
| 372 | 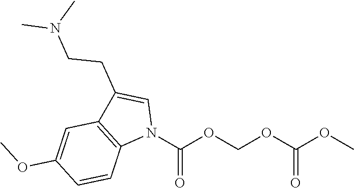 |
| | CN(CCC1═CN(C2═C1C═C(OC)C═C2)C(OCOC(OC)═O)═O)C |
| 373 | 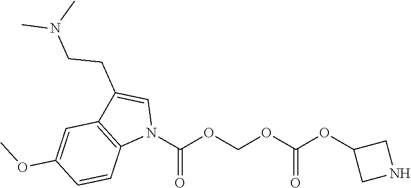 [SMILES: COc1ccc2c(c1)c(CCN(C)C)cn2C(=O)OCOC(=O)OC1CNC1] |
| | CN(CCC1═CN(C2═C1C═C(OC)C═C2)C(OCOC(OC3CNC3)═O)═O)C |
| 374 | 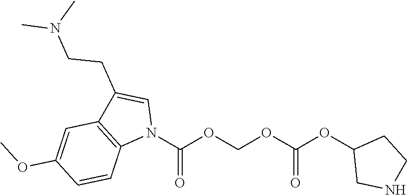 [SMILES: COc1ccc2c(c1)c(CCN(C)C)cn2C(=O)OCOC(=O)OC1CCNC1] |
| | CN(CCC1═CN(C2═C1C═C(OC)C═C2)C(OCOC(OC3CNCC3)═O)═O)C |
| 375 | 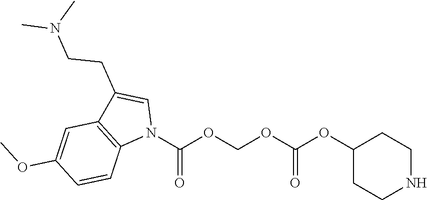 [SMILES: COc1ccc2c(c1)c(CCN(C)C)cn2C(=O)OCOC(=O)OC1CCNCC1] |
| | CN(CCC1═CN(C2═C1C═C(OC)C═C2)C(OCOC(OC3CCNCC3)═O)═O)C |
| 376 | 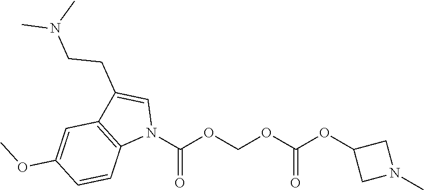 |
| | CN(CCC1═CN(C2═C1C═C(OC)C═C2)C(OCOC(OC3CN(C)C3)═O)═O)C |
| 377 | 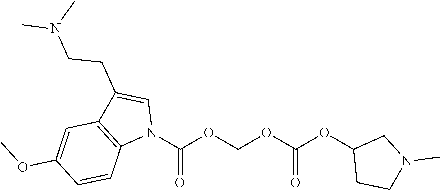 |
| | CN(CCC1═CN(C2═C1C═C(OC)C═C2)C(OCOC(OC3CN(C)CC3)═O)═O)C |
| 378 | 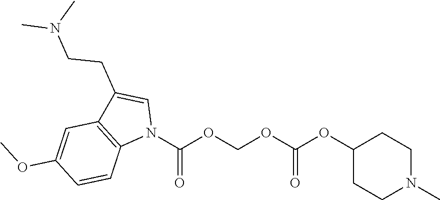 |
| | CN(CCC1═CN(C2═C1C═C(OC)C═C2)C(OCOC(OC3CCN(C)CC3)═O)═O)C |
| 379 | 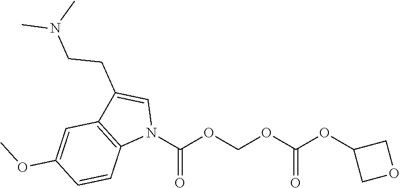 [SMILES: COc1ccc2c(c1)c(CCN(C)C)cn2C(=O)OCOC(=O)OC1COC1] |
| | CN(CCC1═CN(C2═C1C═C(OC)C═C2)C(OCOC(OC3COC3)═O)═O)C |
| 380 | 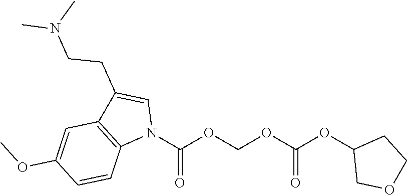 [SMILES: COc1ccc2c(c1)c(CCN(C)C)cn2C(=O)OCOC(=O)OC1CCOC1] |
| | CN(CCC1═CN(C2═C1C═C(OC)C═C2)C(OCOC(OC3COCC3)═O)═O)C |
| 381 | 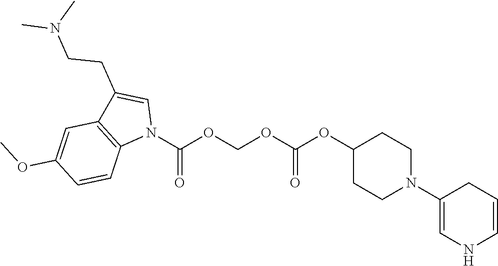 [SMILES: COc1ccc2c(c1)c(CCN(C)C)cn2C(=O)OCOC(=O)OC1CCN(C2=CNC=CC2)CC1] |
| | CN(CCC1═CN(C2═C1C═C(OC)C═C2)C(OCOC(OC3CCN(CC3)C4═CNC═CC4)═O)═O)C |
| 382 | 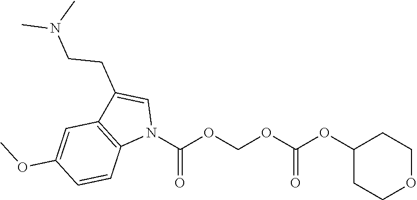 [SMILES: COc1ccc2c(c1)c(CCN(C)C)cn2C(=O)OCOC(=O)OC1CCOCC1] |
| | CN(CCC1═CN(C2═C1C═C(OC)C═C2)C(OCOC(OC3CCOCC3)═O)═O)C |
| 383 | 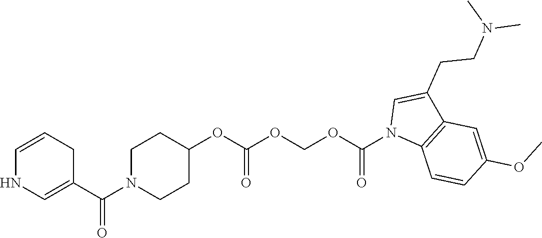 [SMILES: COc1ccc2c(c1)c(CCN(C)C)cn2C(=O)OCOC(=O)OC1CCN(C(=O)C2=CNC=CC2)CC1] |
| | CN(CCC1═CN(C2═C1C═C(OC)C═C2)C(OCOC(OC3CCN(CC3)C(C4═CNC═CC4)═O)═O)═O)C |
| 384 | 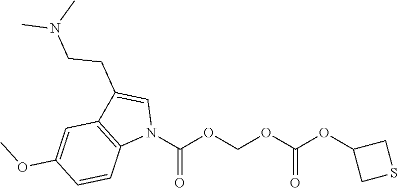 [SMILES: COc1ccc2c(c1)c(CCN(C)C)cn2C(=O)OCOC(=O)OC1CSC1] |
| | CN(CCC1═CN(C2═C1C═C(OC)C═C2)C(OCOC(OC3CSC3)═O)═O)C |
| 385 | 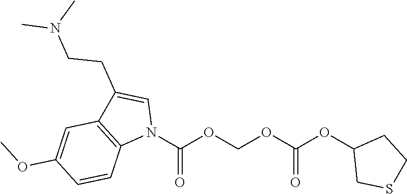 [SMILES: COc1ccc2c(c1)c(CCN(C)C)cn2C(=O)OCOC(=O)OC1CCSC1] |
| | CN(CCC1═CN(C2═C1C═C(OC)C═C2)C(OCOC(OC3CSCC3)═O)═O)C |
| 386 | 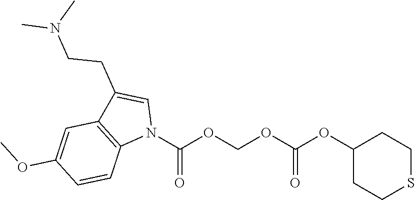 [SMILES: COc1ccc2c(c1)c(CCN(C)C)cn2C(=O)OCOC(=O)OC1CCSCC1] |
| | CN(CCC1═CN(C2═C1C═C(OC)C═C2)C(OCOC(OC3CCSCC3)═O)═O)C |
| 387 | 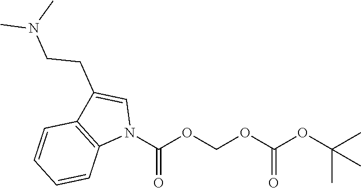 [SMILES: CN(C)CCc1cn(C(=O)OCOC(=O)OC(C)(C)C)c2ccccc12] |
| | CN(CCC1═CN(C2═C1C═CC═C2)C(OCOC(OC(C)(C)C)═O)═O)C |
| 388 | 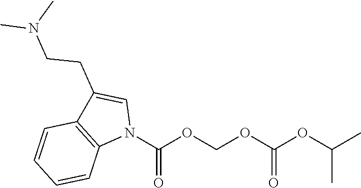 [SMILES: CC(C)OC(=O)OCOC(=O)n1cc(CCN(C)C)c2ccccc21] |
| | CN(CCC1═CN(C2═C1C═CC═C2)C(OCOC(OC(C)C)═O)═O)C |
| 389 | 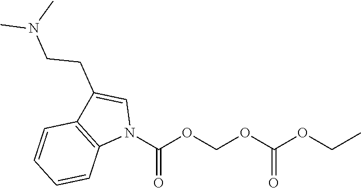 [SMILES: CCOC(=O)OCOC(=O)n1cc(CCN(C)C)c2ccccc21] |
| | CN(CCC1═CN(C2═C1C═CC═C2)C(OCOC(OCC)═O)═O)C |
| 390 | 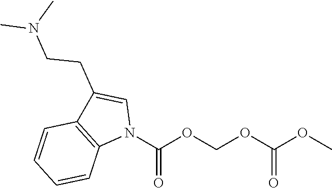 |
| | CN(CCC1═CN(C2═C1C═CC═C2)C(OCOC(OC)═O)═O)C |
| 391 | 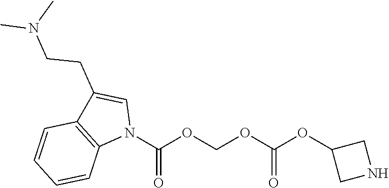 [SMILES: CN(C)CCc1cn(C(=O)OCOC(=O)OC2CNC2)c2ccccc12] |
| | CN(CCC1═CN(C2═C1C═CC═C2)C(OCOC(OC3CNC3)═O)═O)C |
| 392 | 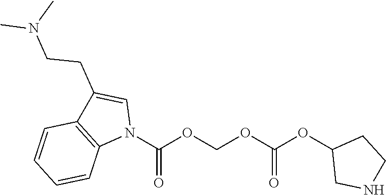 [SMILES: CN(C)CCc1cn(C(=O)OCOC(=O)OC2CCNC2)c2ccccc12] |
| | CN(CCC1═CN(C2═C1C═CC═C2)C(OCOC(OC3CNCC3)═O)═O)C |
| 393 | 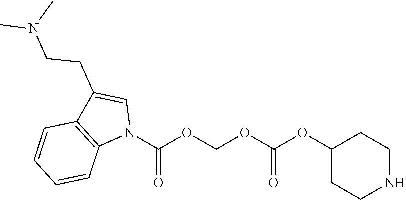 [SMILES: CN(C)CCc1cn(C(=O)OCOC(=O)OC2CCNCC2)c2ccccc12] |
| | CN(CCC1═CN(C2═C1C═CC═C2)C(OCOC(OC3CCNCC3)═O)═O)C |
| 394 | 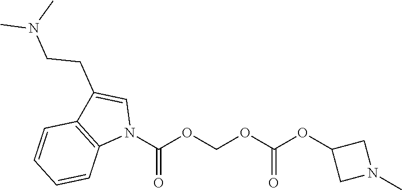 |
| | CN(CCC1═CN(C2═C1C═CC═C2)C(OCOC(OC3CN(C)C3)═O)═O)C |
| 395 | 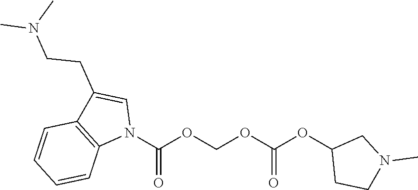 |
| | CN(CCC1═CN(C2═C1C═CC═C2)C(OCOC(OC3CN(C)CC3)═O)═O)C |
| 396 | 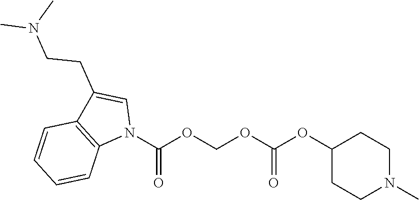 |
| | CN(CCC1═CN(C2═C1C═CC═C2)C(OCOC(OC3CCN(C)CC3)═O)═O)C |
| 397 | 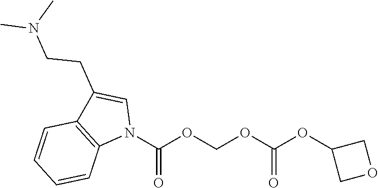 [SMILES: CN(C)CCc1cn(C(=O)OCOC(=O)OC2COC2)c2ccccc12] |
| | CN(CCC1═CN(C2═C1C═CC═C2)C(OCOC(OC3COC3)═O)═O)C |
| 398 | 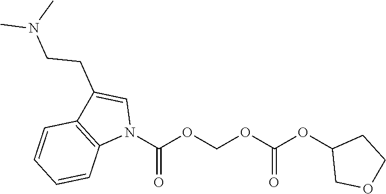 [SMILES: CN(C)CCc1cn(C(=O)OCOC(=O)OC2CCOC2)c2ccccc12] |
| | CN(CCC1═CN(C2═C1C═CC═C2)C(OCOC(OC3COCC3)═O)═O)C |
| 399 | 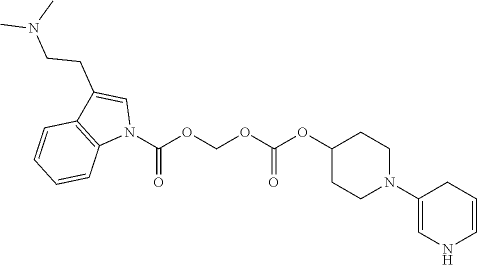 [SMILES: CN(C)CCc1cn(C(=O)OCOC(=O)OC2CCN(C3=CNC=CC3)CC2)c2ccccc12] |
| | CN(CCC1═CN(C2═C1C═CC═C2)C(OCOC(OC3CCN(CC3)C4═CNC═CC4)═O)═O)C |
| 400 | 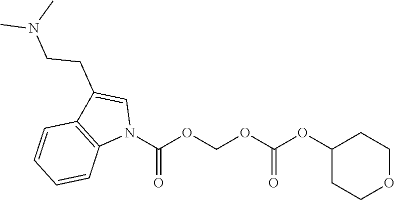 [SMILES: CN(C)CCc1cn(C(=O)OCOC(=O)OC2CCOCC2)c2ccccc12] |
| | CN(CCC1═CN(C2═C1C═CC═C2)C(OCOC(OC3CCOCC3)═O)═O)C |
| 401 | 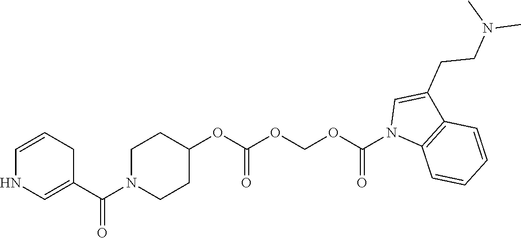 [SMILES: CN(C)CCc1cn(C(=O)OCOC(=O)OC2CCN(C(=O)C3=CNC=CC3)CC2)c2ccccc12] |
| | CN(CCC1═CN(C2═C1C═CC═C2)C(OCOC(OC3CCN(CC3)C(C4═CNC═CC4)═O)═O)═O)C |
| 402 | 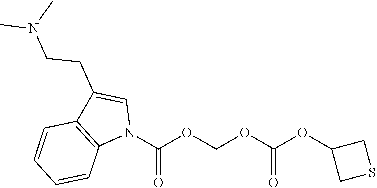 [SMILES: CN(C)CCc1cn(C(=O)OCOC(=O)OC2CSC2)c2ccccc12] |
| | CN(CCC1═CN(C2═C1C═CC═C2)C(OCOC(OC3CSC3)═O)═O)C |
| 403 | 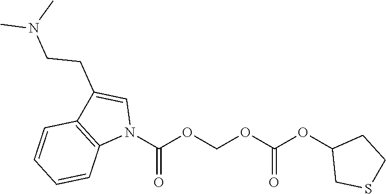 [SMILES: CN(C)CCc1cn(C(=O)OCOC(=O)OC2CCSC2)c2ccccc12] |
| | CN(CCC1═CN(C2═C1C═CC═C2)C(OCOC(OC3CSCC3)═O)═O)C |
| 404 | 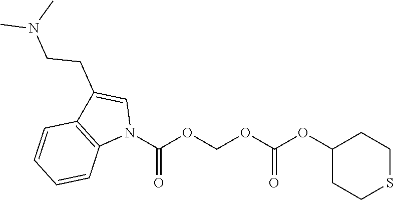 [SMILES: CN(C)CCc1cn(C(=O)OCOC(=O)OC2CCSCC2)c2ccccc12] |
| | CN(CCC1═CN(C2═C1C═CC═C2)C(OCOC(OC3CCSCC3)═O)═O)C |
| 405 | 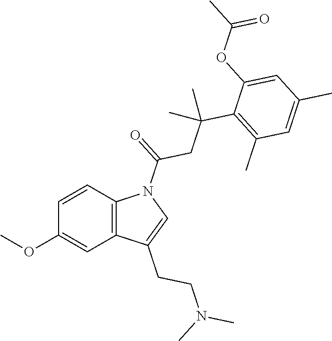 |
| | CN(CCC1═CN(C2═C1C═C(OC)C═C2)C(CC(C)(C3═C(C═C(C═C3OC(C)═O)C)C)C)═O)C |
| 406 | 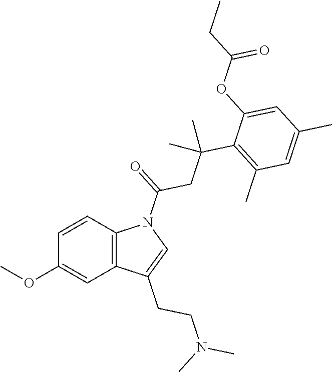 |
| | CN(CCC1═CN(C2═C1C═C(OC)C═C2)C(CC(C)(C3═C(C═C(C═C3OC(CC)═O)C)C)C)═O)C |
| 407 | 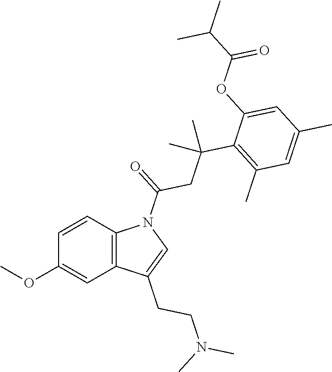 |
| | CN(CCC1═CN(C2═C1C═C(OC)C═C2)C(CC(C)(C3═C(C═C(C═C3OC(C(C)C)═O)C)C)C)═O)C |
| 408 | 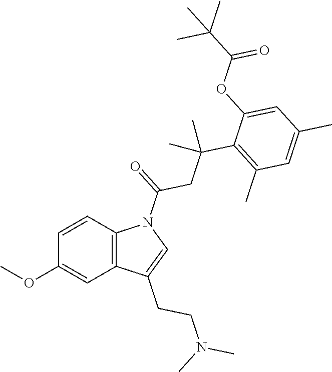 |
| | CN(CCC1═CN(C2═C1C═C(OC)C═C2)C(CC(C)(C3═C(C═C(C═C3OC(C(C)(C)C)═O)C)C)C)═O)C |
| 409 | 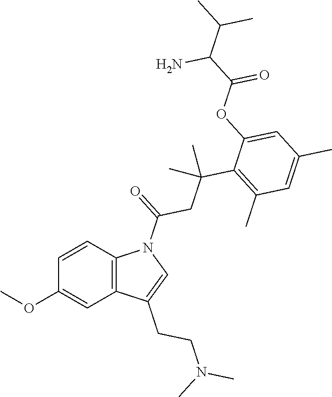 |
| | CN(CCC1═CN(C2═C1C═C(OC)C═C2)C(CC(C)(C3═C(C═C(C═C3OC(C(C(C)C)N)═O)C)C)C)═O)C |
| 410 | 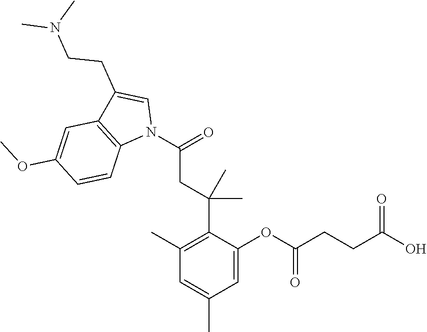 [SMILES: COc1ccc2c(c1)c(CCN(C)C)cn2C(=O)CC(C)(C)c1c(C)cc(C)cc1OC(=O)CCC(=O)O] |
| | CN(CCC1═CN(C2═C1C═C(OC)C═C2)C(CC(C)(C3═C(C═C(C═C3OC(CCC(O)═O)═O)C)C)C)═O)C |
| 411 | 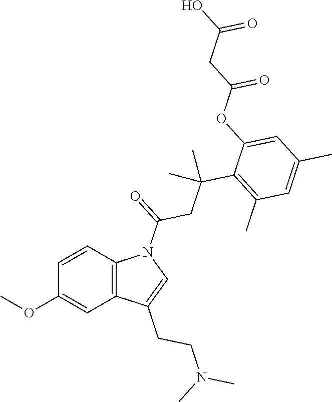 |
| | CN(CCC1═CN(C2═C1C═C(OC)C═C2)C(CC(C)(C3═C(C═C(C═C3OC(CC(O)═O)═O)C)C)C)═O)C |
| 412 | 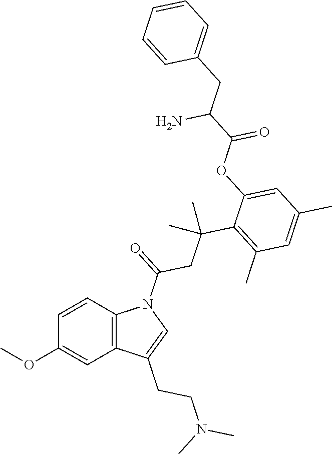 |
| | CN(CCC1═CN(C2═C1C═C(OC)C═C2)C(CC(C)(C3═C(C═C(C═C3OC(C(CC4═CC═CC═C4)N)═O)C)C)C)═O)C |
| 413 | 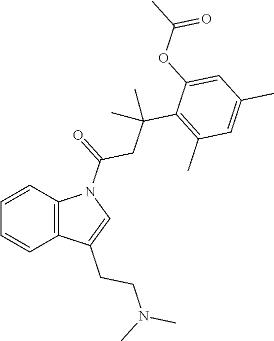 [SMILES: CC(=O)Oc1cc(C)cc(C)c1C(C)(C)CC(=O)n1cc(CCN(C)C)c2ccccc21] |
| | CN(CCC1═CN(C2═C1C═CC═C2)C(CC(C)(C3═C(C═C(C═C3OC(C)═O)C)C)C)═O)C |
| 414 | 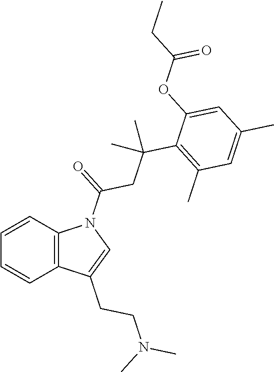 [SMILES: CCC(=O)Oc1cc(C)cc(C)c1C(C)(C)CC(=O)n1cc(CCN(C)C)c2ccccc21] |
| | CN(CCC1═CN(C2═C1C═CC═C2)C(CC(C)(C3═C(C═C(C═C3OC(CC)═O)C)C)C)═O)C |
| 415 | 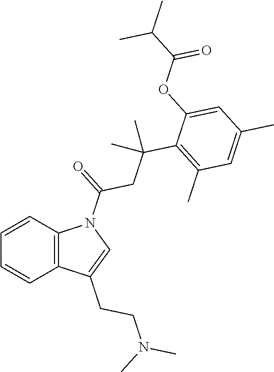 [SMILES: Cc1cc(C)c(C(C)(C)CC(=O)n2cc(CCN(C)C)c3ccccc32)c(OC(=O)C(C)C)c1] |
| | CN(CCC1═CN(C2═C1C═CC═C2)C(CC(C)(C3═C(C═C(C═C3OC(C(C)C)═O)C)C)C)═O)C |
| 416 | 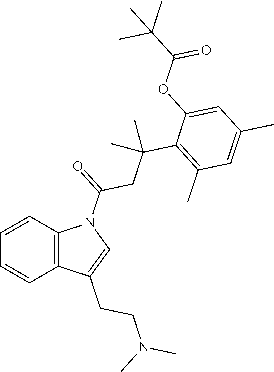 [SMILES: Cc1cc(C)c(C(C)(C)CC(=O)n2cc(CCN(C)C)c3ccccc32)c(OC(=O)C(C)(C)C)c1] |
| | CN(CCC1═CN(C2═C1C═CC═C2)C(CC(C)(C3═C(C═C(C═C3OC(C(C)(C)C)═O)C)C)C)═O)C |
| 417 | 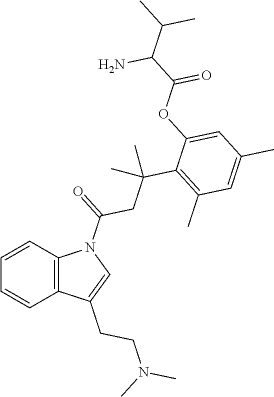 [SMILES: Cc1cc(C)c(C(C)(C)CC(=O)n2cc(CCN(C)C)c3ccccc32)c(OC(=O)C(N)C(C)C)c1] |
| | CN(CCC1═CN(C2═C1C═CC═C2)C(CC(C)(C3═C(C═C(C═C3OC(C(C(C)C)N)═O)C)C)C)═O)C |
| 418 | 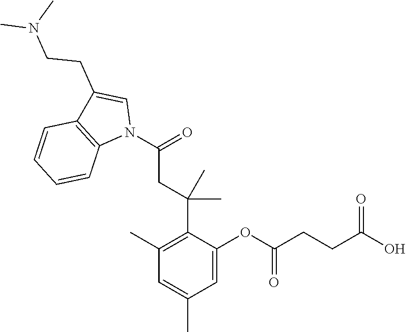 [SMILES: Cc1cc(C)c(C(C)(C)CC(=O)n2cc(CCN(C)C)c3ccccc32)c(OC(=O)CCC(=O)O)c1] |
| | CN(CCC1═CN(C2═C1C═CC═C2)C(CC(C)(C3═C(C═C(C═C3OC(CCC(O)═O)═O)C)C)C)═O)C |
| 419 | 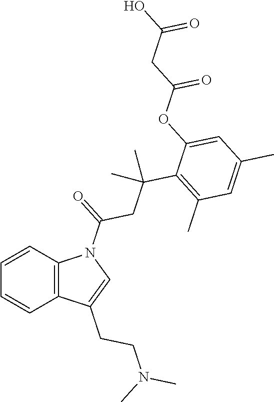 [SMILES: Cc1cc(C)c(C(C)(C)CC(=O)n2cc(CCN(C)C)c3ccccc32)c(OC(=O)CC(=O)O)c1] |
| | CN(CCC1═CN(C2═C1C═CC═C2)C(CC(C)(C3═C(C═C(C═C3OC(CC(O)═O)═O)C)C)C)═O)C |
| 420 | 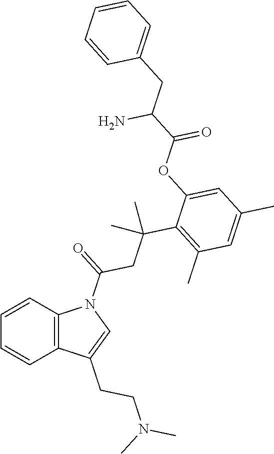 [SMILES: Cc1cc(C)c(C(C)(C)CC(=O)n2cc(CCN(C)C)c3ccccc32)c(OC(=O)C(N)Cc2ccccc2)c1] |
| | CN(CCC1═CN(C2═C1C═CC═C2)C(CC(C)(C3═C(C═C(C═C3OC(C(CC4═CC═CC═C4)N)═O)C)C)C)═O)C |
| 421 | 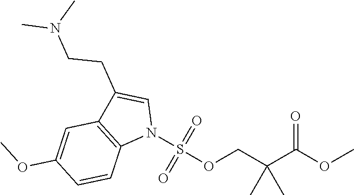 |
| | CN(CCC1═CN(C2═C1C═C(OC)C═C2)S(OCC(C(OC)═O)(C)C)(═O)═O)C |
| 422 | 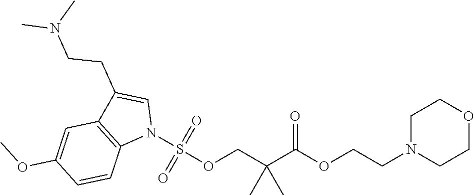 [SMILES: COc1ccc2c(c1)c(CCN(C)C)cn2S(=O)(=O)OCC(C)(C)C(=O)OCCN1CCOCC1] |
| | CN(CCC1═CN(C2═C1C═C(OC)C═C2)S(OCC(C)(C)C(OCCN3CCOCC3)═O)(═O)═O)C |
| 423 | 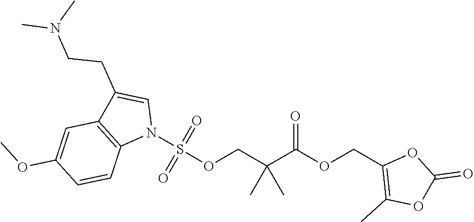 [SMILES: COc1ccc2c(c1)c(CCN(C)C)cn2S(=O)(=O)OCC(C)(C)C(=O)OCc1oc(=O)oc1C] |
| | CN(CCC1═CN(C2═C1C═C(OC)C═C2)S(OCC(C(OCC3═C(OC(O3)═O)C)═O)(C)C)(═O)═O)C |
| 424 | 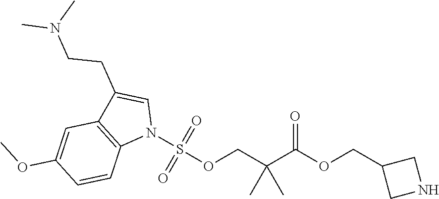 [SMILES: COc1ccc2c(c1)c(CCN(C)C)cn2S(=O)(=O)OCC(C)(C)C(=O)OCC1CNC1] |
| | CN(CCC1═CN(C2═C1C═C(OC)C═C2)S(OCC(C(OCC3CNC3)═O)(C)C)(═O)═O)C |
| 425 | 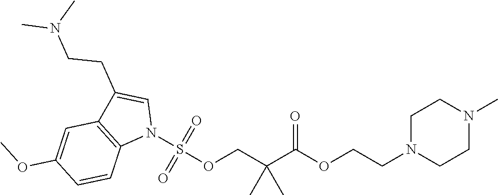 |
| | CN(CCC1═CN(C2═C1C═C(OC)C═C2)S(OCC(C)(C)C(OCCN3CCN(CC3)C)═O)(═O)═O)C |
| 426 | 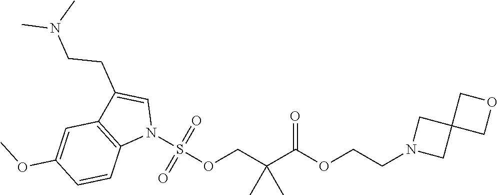 [SMILES: COc1ccc2c(c1)c(CCN(C)C)cn2S(=O)(=O)OCC(C)(C)C(=O)OCCN1CC2(COC2)C1] |
| | CN(CCC1═CN(C2═C1C═C(OC)C═C2)S(OCC(C)(C)C(OCCN3CC4(C3)COC4)═O)(═O)═O)C |
| 427 | 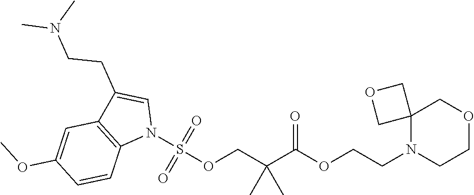 [SMILES: COc1ccc2c(c1)c(CCN(C)C)cn2S(=O)(=O)OCC(C)(C)C(=O)OCCN1CCOCC12COC2] |
| | CN(CCC1═CN(C2═C1C═C(OC)C═C2)S(OCC(C)(C)C(OCCN3CCOCC34COC4)═O)(═O)═O)C |
| 428 | 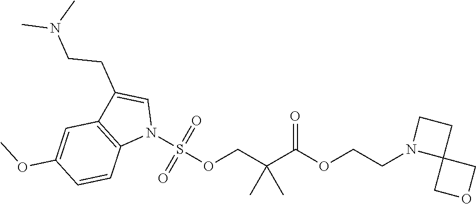 [SMILES: COc1ccc2c(c1)c(CCN(C)C)cn2S(=O)(=O)OCC(C)(C)C(=O)OCCN1CCC12COC2] |
| | CN(CCC1═CN(C2═C1C═C(OC)C═C2)S(OCC(C)(C)C(OCCN3CCC34COC4)═O)(═O)═O)C |
| 429 | 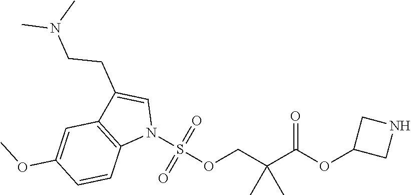 [SMILES: COc1ccc2c(c1)c(CCN(C)C)cn2S(=O)(=O)OCC(C)(C)C(=O)OC1CNC1] |
| | CN(CCC1═CN(C2═C1C═C(OC)C═C2)S(OCC(C(OC3CNC3)═O)(C)C)(═O)═O)C |
| 430 | 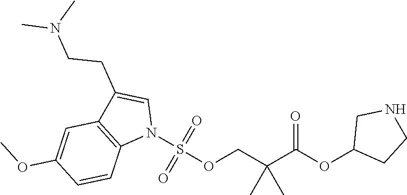 [SMILES: COc1ccc2c(c1)c(CCN(C)C)cn2S(=O)(=O)OCC(C)(C)C(=O)OC1CCNC1] |
| | CN(CCC1═CN(C2═C1C═C(OC)C═C2)S(OCC(C(OC3CNCC3)═O)(C)C)(═O)═O)C |
| 431 | 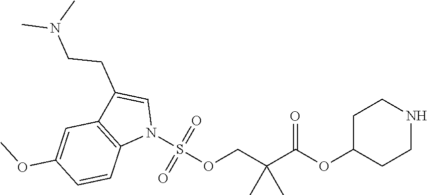 [SMILES: COc1ccc2c(c1)c(CCN(C)C)cn2S(=O)(=O)OCC(C)(C)C(=O)OC1CCNCC1] |
| | CN(CCC1═CN(C2═C1C═C(OC)C═C2)S(OCC(C(OC3CCNCC3)═O)(C)C)(═O)═O)C |
| 432 | 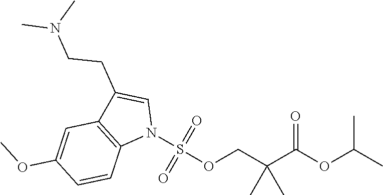 [SMILES: COc1ccc2c(c1)c(CCN(C)C)cn2S(=O)(=O)OCC(C)(C)C(=O)OC(C)C] |
| | CN(CCC1═CN(C2═C1C═C(OC)C═C2)S(OCC(C(OC(C)C)═O)(C)C)(═O)═O)C |
| 433 | 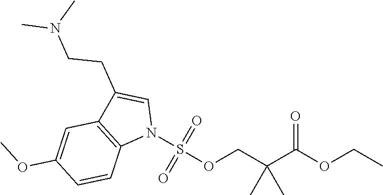 [SMILES: CCOC(=O)C(C)(C)COS(=O)(=O)n1cc(CCN(C)C)c2cc(OC)ccc21] |
| | CN(CCC1═CN(C2═C1C═C(OC)C═C2)S(OCC(C(OCC)═O)(C)C)(═O)═O)C |
| 434 | 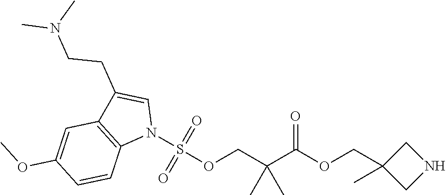 [SMILES: COc1ccc2c(c1)c(CCN(C)C)cn2S(=O)(=O)OCC(C)(C)C(=O)OCC1(C)CNC1] |
| | CN(CCC1═CN(C2═C1C═C(OC)C═C2)S(OCC(C(OCC3(C)CNC3)═O)(C)C)(═O)═O)C |
| 435 | 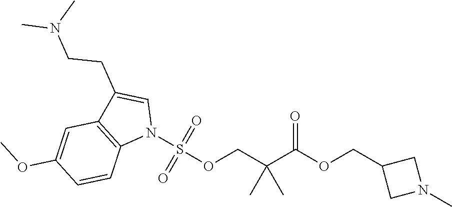 |
| | CN(CCC1═CN(C2═C1C═C(OC)C═C2)S(OCC(C(OCC3CN(C)C3)═O)(C)C)(═O)═O)C |
| 436 | 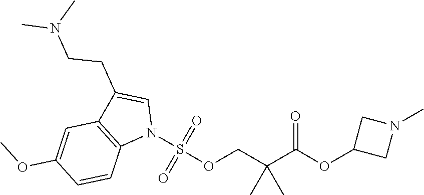 |
| | CN(CCC1═CN(C2═C1C═C(OC)C═C2)S(OCC(C(OC3CN(C)C3)═O)(C)C)(═O)═O)C |
| 437 | 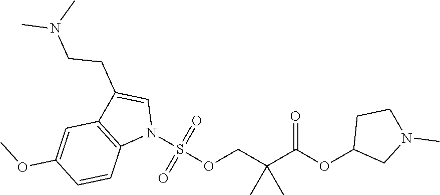 |
| | CN(CCC1═CN(C2═C1C═C(OC)C═C2)S(OCC(C(OC3CN(C)CC3)═O)(C)C)(═O)═O)C |
| 438 | 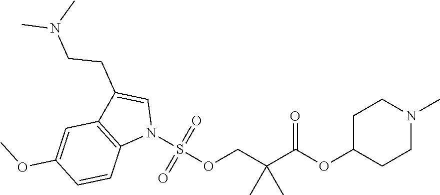 |
| | CN(CCC1═CN(C2═C1C═C(OC)C═C2)S(OCC(C(OC3CCN(C)CC3)═O)(C)C)(═O)═O)C |
| 439 | 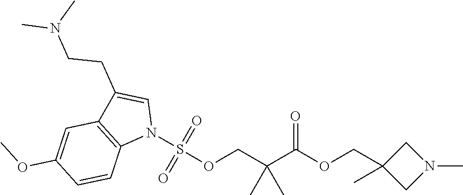 |
| | CN(CCC1═CN(C2═C1C═C(OC)C═C2)S(OCC(C(OCC3(C)CN(C)C3)═O)(C)C)(═O)═O)C |
| 440 | 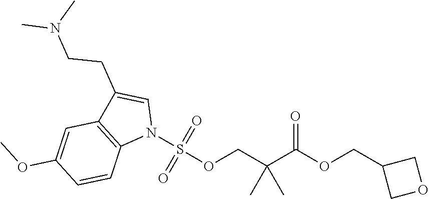 [SMILES: COc1ccc2c(c1)c(CCN(C)C)cn2S(=O)(=O)OCC(C)(C)C(=O)OCC1COC1] |
| | CN(CCC1═CN(C2═C1C═C(OC)C═C2)S(OCC(C(OCC3COC3)═O)(C)C)(═O)═O)C |
| 441 | 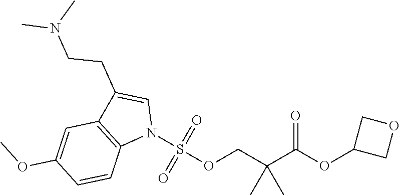 [SMILES: COc1ccc2c(c1)c(CCN(C)C)cn2S(=O)(=O)OCC(C)(C)C(=O)OC1COC1] |
| | CN(CCC1═CN(C2═C1C═C(OC)C═C2)S(OCC(C(OC3COC3)═O)(C)C)(═O)═O)C |
| 442 | 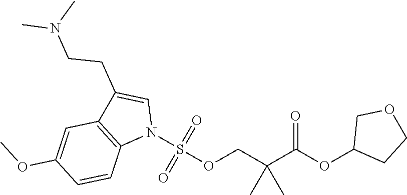 [SMILES: COc1ccc2c(c1)c(CCN(C)C)cn2S(=O)(=O)OCC(C)(C)C(=O)OC1CCOC1] |
| | CN(CCC1═CN(C2═C1C═C(OC)C═C2)S(OCC(C(OC3COCC3)═O)(C)C)(═O)═O)C |
| 443 | 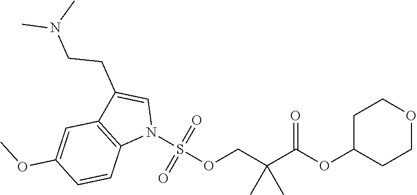 [SMILES: COc1ccc2c(c1)c(CCN(C)C)cn2S(=O)(=O)OCC(C)(C)C(=O)OC1CCOCC1] |
| | CN(CCC1═CN(C2═C1C═C(OC)C═C2)S(OCC(C(OC3CCOCC3)═O)(C)C)(═O)═O)C |
| 444 | 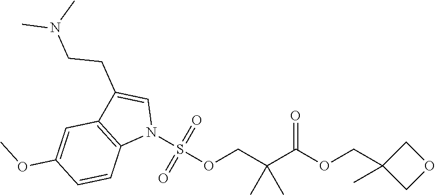 [SMILES: COc1ccc2c(c1)c(CCN(C)C)cn2S(=O)(=O)OCC(C)(C)C(=O)OCC1(C)COC1] |
| | CN(CCC1═CN(C2═C1C═C(OC)C═C2)S(OCC(C(OCC3(C)COC3)═O)(C)C)(═O)═O)C |
| 445 | 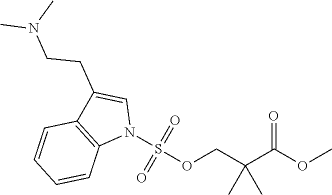 |
| | CN(CCC1═CN(C2═C1C═CC═C2)S(OCC(C(OC)═O)(C)C)(═O)═O)C |
| 446 | 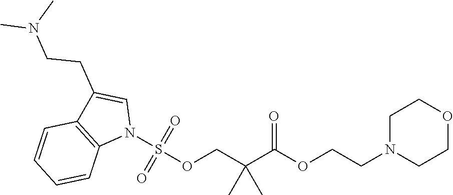 [SMILES: CN(C)CCc1cn(S(=O)(=O)OCC(C)(C)C(=O)OCCN2CCOCC2)c2ccccc12] |
| | CN(CCC1═CN(C2═C1C═CC═C2)S(OCC(C)(C)C(OCCN3CCOCC3)═O)(═O)═O)C |
| 447 | 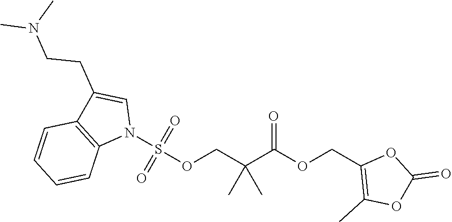 [SMILES: Cc1oc(=O)oc1COC(=O)C(C)(C)COS(=O)(=O)n1cc(CCN(C)C)c2ccccc21] |
| | CN(CCC1═CN(C2═C1C═CC═C2)S(OCC(C(OCC3═C(OC(O3)═O)C)═O)(C)C)(═O)═O)C |
| 448 | 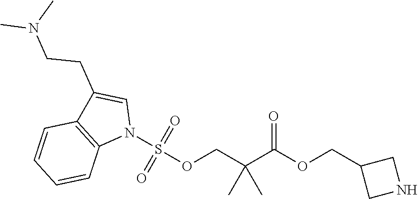 [SMILES: CN(C)CCc1cn(S(=O)(=O)OCC(C)(C)C(=O)OCC2CNC2)c2ccccc12] |
| | CN(CCC1═CN(C2═C1C═CC═C2)S(OCC(C(OCC3CNC3)═O)(C)C)(═O)═O)C |
| 449 | 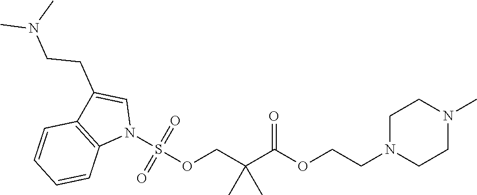 |
| | CN(CCC1═CN(C2═C1C═CC═C2)S(OCC(C)(C)C(OCCN3CCN(CC3)C)═O)(═O)═O)C |
| 450 | 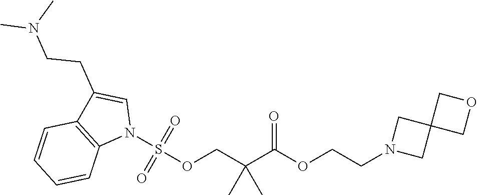 [SMILES: CN(C)CCc1cn(S(=O)(=O)OCC(C)(C)C(=O)OCCN2CC3(COC3)C2)c2ccccc12] |
| | CN(CCC1═CN(C2═C1C═CC═C2)S(OCC(C)(C)C(OCCN3CC4(C3)COC4)═O)(═O)═O)C |
| 451 | 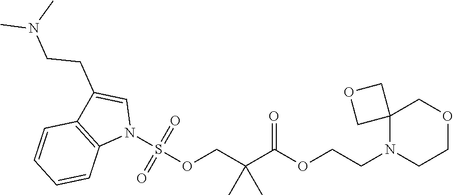 [SMILES: CN(C)CCc1cn(S(=O)(=O)OCC(C)(C)C(=O)OCCN2CCOCC23COC3)c2ccccc12] |
| | CN(CCC1═CN(C2═C1C═CC═C2)S(OCC(C)(C)C(OCCN3CCOCC34COC4)═O)(═O)═O)C |
| 452 | 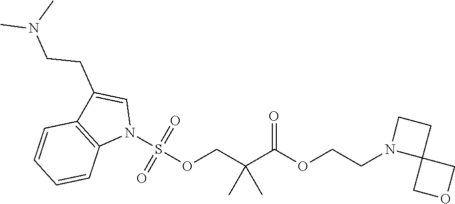 [SMILES: CN(C)CCc1cn(S(=O)(=O)OCC(C)(C)C(=O)OCCN2CCC23COC3)c2ccccc12] |
| | CN(CCC1═CN(C2═C1C═CC═C2)S(OCC(C)(C)C(OCCN3CCC34COC4)═O)(═O)═O)C |
| 453 | 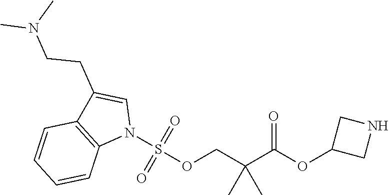 [SMILES: CN(C)CCc1cn(S(=O)(=O)OCC(C)(C)C(=O)OC2CNC2)c2ccccc12] |
| | CN(CCC1═CN(C2═C1C═CC═C2)S(OCC(C(OC3CNC3)═O)(C)C)(═O)═O)C |
| 454 | 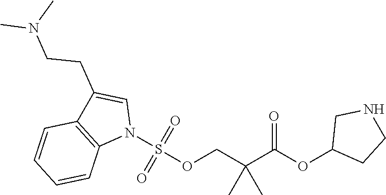 [SMILES: CN(C)CCc1cn(S(=O)(=O)OCC(C)(C)C(=O)OC2CCNC2)c2ccccc12] |
| | CN(CCC1═CN(C2═C1C═CC═C2)S(OCC(C(OC3CNCC3)═O)(C)C)(═O)═O)C |
| 455 | 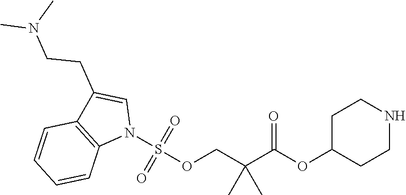 [SMILES: CN(C)CCc1cn(S(=O)(=O)OCC(C)(C)C(=O)OC2CCNCC2)c2ccccc12] |
| | CN(CCC1═CN(C2═C1C═CC═C2)S(OCC(C(OC3CCNCC3)═O)(C)C)(═O)═O)C |
| 456 | 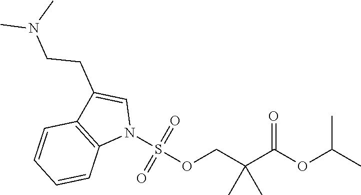 [SMILES: CC(C)OC(=O)C(C)(C)COS(=O)(=O)n1cc(CCN(C)C)c2ccccc21] |
| | CN(CCC1═CN(C2═C1C═CC═C2)S(OCC(C(OC(C)C)═O)(C)C)(═O)═O)C |
| 457 | 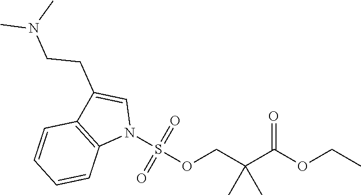 [SMILES: CCOC(=O)C(C)(C)COS(=O)(=O)n1cc(CCN(C)C)c2ccccc21] |
| | CN(CCC1═CN(C2═C1C═CC═C2)S(OCC(C(OCC)═O)(C)C)(═O)═O)C |
| 458 | 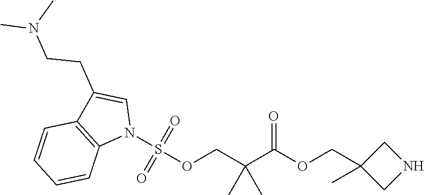 [SMILES: CN(C)CCc1cn(S(=O)(=O)OCC(C)(C)C(=O)OCC2(C)CNC2)c2ccccc12] |
| | CN(CCC1═CN(C2═C1C═CC═C2)S(OCC(C(OCC3(C)CNC3)═O)(C)C)(═O)═O)C |
| 459 | 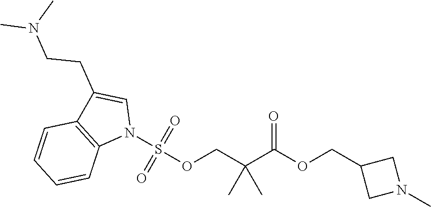 |
| | CN(CCC1═CN(C2═C1C═CC═C2)S(OCC(C(OCC3CN(C)C3)═O)(C)C)(═O)═O)C |
| 460 | 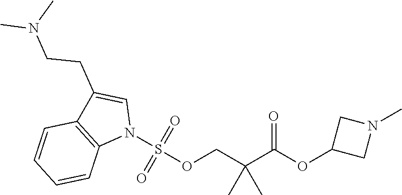 |
| | CN(CCC1═CN(C2═C1C═CC═C2)S(OCC(C(OC3CN(C)C3)═O)(C)C)(═O)═O)C |
| 461 | 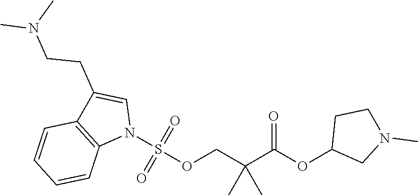 |
| | CN(CCC1═CN(C2═C1C═CC═C2)S(OCC(C(OC3CN(C)CC3)═O)(C)C)(═O)═O)C |
| 462 | 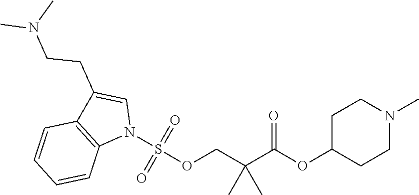 |
| | CN(CCC1═CN(C2═C1C═CC═C2)S(OCC(C(OC3CCN(C)CC3)═O)(C)C)(═O)═O)C |
| 463 | 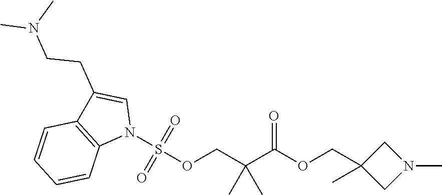 |
| | CN(CCC1═CN(C2═C1C═CC═C2)S(OCC(C(OCC3(C)CN(C)C3)═O)(C)C)(═O)═O)C |
| 464 | 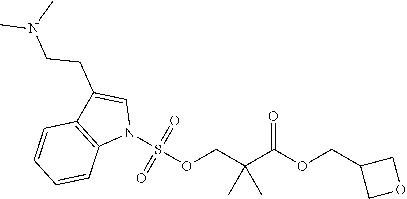 [SMILES: CN(C)CCc1cn(S(=O)(=O)OCC(C)(C)C(=O)OCC2COC2)c2ccccc12] |
| | CN(CCC1═CN(C2═C1C═CC═C2)S(OCC(C(OCC3COC3)═O)(C)C)(═O)═O)C |
| 465 | 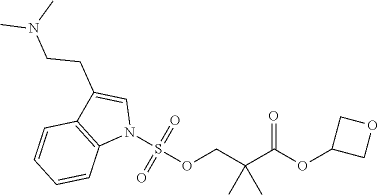 [SMILES: CN(C)CCc1cn(S(=O)(=O)OCC(C)(C)C(=O)OC2COC2)c2ccccc12] |
| | CN(CCC1═CN(C2═C1C═CC═C2)S(OCC(C(OC3COC3)═O)(C)C)(═O)═O)C |
| 466 | 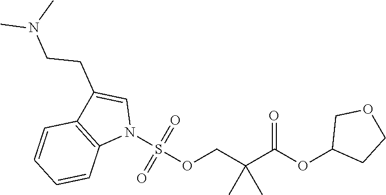 [SMILES: CN(C)CCc1cn(S(=O)(=O)OCC(C)(C)C(=O)OC2CCOC2)c2ccccc12] |
| | CN(CCC1═CN(C2═C1C═CC═C2)S(OCC(C(OC3COCC3)═O)(C)C)(═O)═O)C |
| 467 | 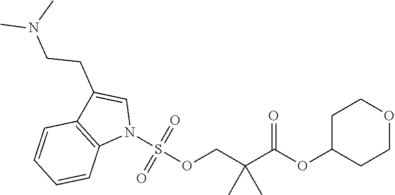 [SMILES: CN(C)CCc1cn(S(=O)(=O)OCC(C)(C)C(=O)OC2CCOCC2)c2ccccc12] |
| | CN(CCC1═CN(C2═C1C═CC═C2)S(OCC(C(OC3OCCOCC3)═O)(C)C)(═O)═O)C |
| 468 | 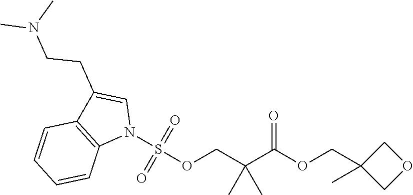 [SMILES: CN(C)CCc1cn(S(=O)(=O)OCC(C)(C)C(=O)OCC2(C)COC2)c2ccccc12] |
| | CN(CCC1═CN(C2═C1C═CC═C2)S(OCC(C(OCC3(C)COC3)═O)(C)C)(═O)═O)C |
| 469 | 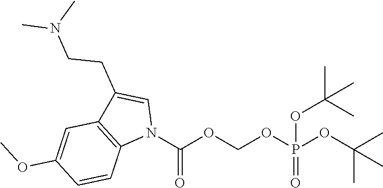 [SMILES: COc1ccc2c(c1)c(CCN(C)C)cn2C(=O)OCOP(=O)(OC(C)(C)C)OC(C)(C)C] |
| | CN(CCC1═CN(C2═C1C═C(OC)C═C2)C(OCOP(OC(C)(C)C)(OC(C)(C)C)═O)═O)C |
| 470 | 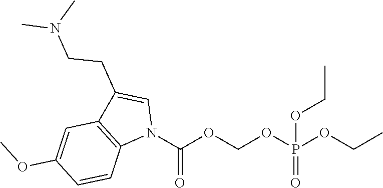 [SMILES: CCOP(=O)(OCC)OCOC(=O)n1cc(CCN(C)C)c2cc(OC)ccc21] |
| | CN(CCC1═CN(C2═C1C═C(OC)C═C2)C(OCOP(OCC)(OCC)═O)═O)C |
| 471 | 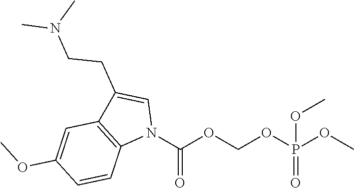 |
| | CN(CCC1═CN(C2═C1C═C(OC)C═C2)C(OCOP(OC)(OC)═O)═O)C |
| 472 | 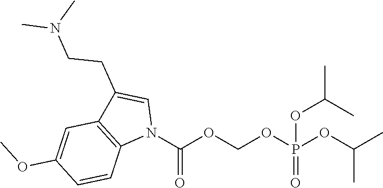 [SMILES: COc1ccc2c(c1)c(CCN(C)C)cn2C(=O)OCOP(=O)(OC(C)C)OC(C)C] |
| | CN(CCC1═CN(C2═C1C═C(OC)C═C2)C(OCOP(OC(C)C)(OC(C)C)═O)═O)C |
| 473 | 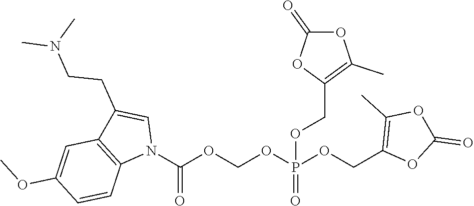 [SMILES: COc1ccc2c(c1)c(CCN(C)C)cn2C(=O)OCOP(=O)(OCc1oc(=O)oc1C)OCc1oc(=O)oc1C] |
| | CN(CCC1═CN(C2═C1C═C(OC)C═C2)C(OCOP(OCC3═C(OC(O3)═O)C)(OCC4═C(OC(O4)═O)C)═O)═O)C |
| 474 | 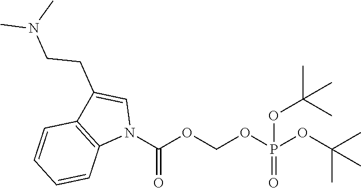 [SMILES: CN(C)CCc1cn(C(=O)OCOP(=O)(OC(C)(C)C)OC(C)(C)C)c2ccccc12] |
| | CN(CCC1═CN(C2═C1C═CC═C2)C(OCOP(OC(C)(C)C)(OC(C)(C)C)═O)═O)C |
| 475 | 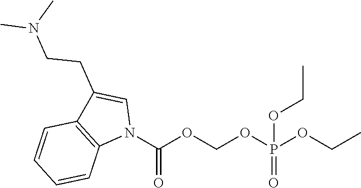 [SMILES: CCOP(=O)(OCC)OCOC(=O)n1cc(CCN(C)C)c2ccccc21] |
| | CN(CCC1═CN(C2═C1C═CC═C2)C(OCOP(OCC)(OCC)═O)═O)C |
| 476 | 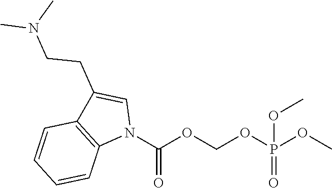 |
| | CN(CCC1═CN(C2═C1C═CC═C2)C(OCOP(OC)(OC)═O)═O)C |
| 477 | 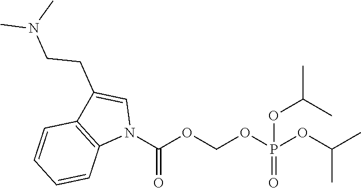 [SMILES: CC(C)OP(=O)(OCOC(=O)n1cc(CCN(C)C)c2ccccc21)OC(C)C] |
| | CN(CCC1═CN(C2═C1C═CC═C2)C(OCOP(OC(C)C)(OC(C)C)═O)═O)C |
| 478 | 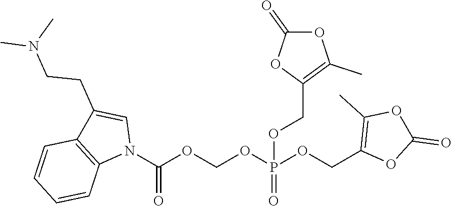 [SMILES: Cc1oc(=O)oc1COP(=O)(OCOC(=O)n1cc(CCN(C)C)c2ccccc21)OCc1oc(=O)oc1C] |
| | CN(CCC1═CN(C2═C1C═CC═C2)C(OCOP(OCC3═C(OC(O3)═O)C)(OCC4═C(OC(O4)═O)C)═O)═O)C |
| 479 | 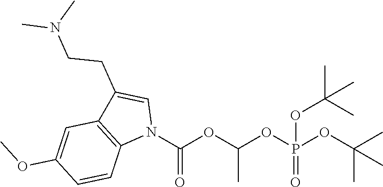 [SMILES: COc1ccc2c(c1)c(CCN(C)C)cn2C(=O)OC(C)OP(=O)(OC(C)(C)C)OC(C)(C)C] |
| | CN(CCC1═CN(C2═C1C═C(OC)C═C2)C(OC(C)OP(OC(C)(C)C)(OC(C)(C)C)═O)═O)C |
| 480 | 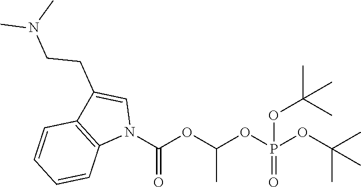 [SMILES: CC(OC(=O)n1cc(CCN(C)C)c2ccccc21)OP(=O)(OC(C)(C)C)OC(C)(C)C] |
| | CN(CCC1═CN(C2═C1C═CC═C2)C(OC(C)OP(OC(C)(C)C)(OC(C)(C)C)═O)═O)C |
| 481 | 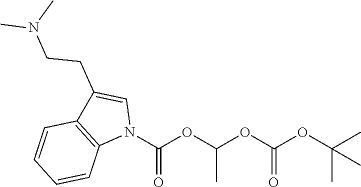 [SMILES: CC(OC(=O)OC(C)(C)C)OC(=O)n1cc(CCN(C)C)c2ccccc21] |
| | CN(CCC1═CN(C2═C1C═CC═C2)C(OC(C)OC(OC(C)(C)C)═O)═O)C |
| 482 | 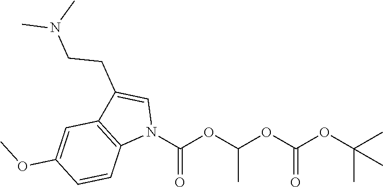 [SMILES: COc1ccc2c(c1)c(CCN(C)C)cn2C(=O)OC(C)OC(=O)OC(C)(C)C] |
| | CN(CCC1═CN(C2═C1C═C(OC)C═C2)C(OC(C)OC(OC(C)(C)C)═O)═O)C |
| 483 | 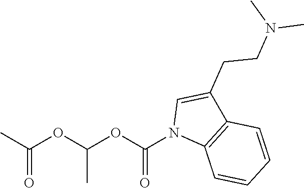 [SMILES: CC(=O)OC(C)OC(=O)n1cc(CCN(C)C)c2ccccc21] |
| | CN(CCC1═CN(C2═C1C═CC═C2)C(OC(OC(C)═O)C)═O)C |
| 484 | 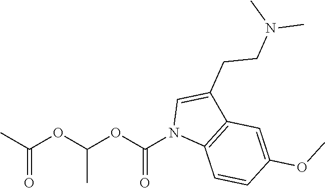 [SMILES: COc1ccc2c(c1)c(CCN(C)C)cn2C(=O)OC(C)OC(C)=O] |
| | CN(CCC1═CN(C2═C1C═C(OC)C═C2)C(OC(OC(C)═O)C)═O)C |
| 485 | 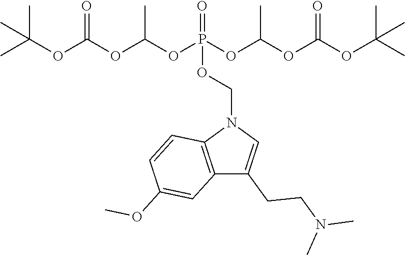 [SMILES: COc1ccc2c(c1)c(CCN(C)C)cn2COP(=O)(OC(C)OC(=O)OC(C)(C)C)OC(C)OC(=O)OC(C)(C)C] |
| | CN(CCC1═CN(C2═C1C═C(OC)C═C2)COP(OC(C)OC(OC(C)(C)C)═O)(OC(C)OC(OC(C)(C)C)═O)═O)C |
| 486 | 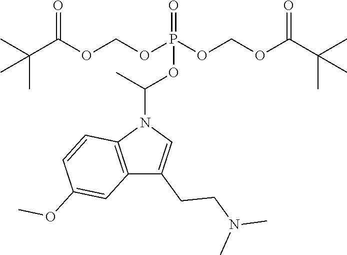 [SMILES: COc1ccc2c(c1)c(CCN(C)C)cn2C(C)OP(=O)(OCOC(=O)C(C)(C)C)OCOC(=O)C(C)(C)C] |
| | CN(CCC1═CN(C2═C1C═C(OC)C═C2)C(C)OP(OCOC(C(C)(C)C)═O)(OCOC(C(C)(C)C)═O)═O)C |
| 487 | 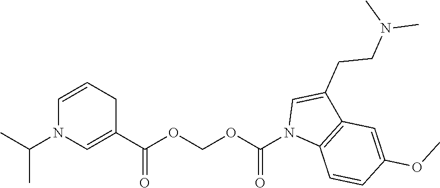 [SMILES: COc1ccc2c(c1)c(CCN(C)C)cn2C(=O)OCOC(=O)C1=CN(C(C)C)C=CC1] |
| | CN(CCC1═CN(C2═C1C═C(OC)C═C2)C(OCOC(C3═CN(C═CC3)C(C)C)═O)═O)C |
| 488 | 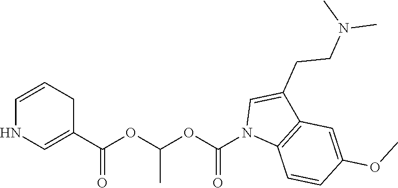 [SMILES: COc1ccc2c(c1)c(CCN(C)C)cn2C(=O)OC(C)OC(=O)C1=CNC=CC1] |
| | CN(CCC1═CN(C2═C1C═C(OC)C═C2)C(OC(C)OC(C3═CNC═CC3)═O)═O)C |
| 489 | 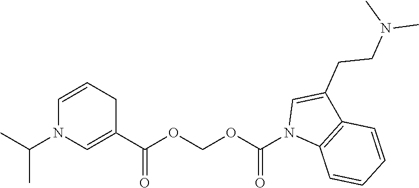 [SMILES: CC(C)N1C=CCC(C(=O)OCOC(=O)n2cc(CCN(C)C)c3ccccc32)=C1] |
| | CN(CCC1═CN(C2═C1C═CC═C2)C(OCOC(C3═CN(C═CC3)C(C)C)═O)═O)C |
| 490 | 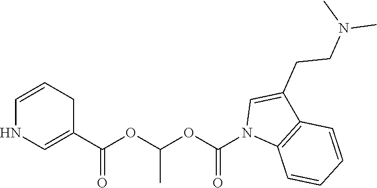 [SMILES: CC(OC(=O)C1=CNC=CC1)OC(=O)n1cc(CCN(C)C)c2ccccc21] |
| | CN(CCC1═CN(C2═C1C═CC═C2)C(OC(C)OC(C3═CNC═CC3)═O)═O)C |
| 491 | 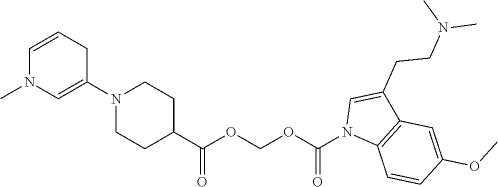 |
| | CN(CCC1═CN(C2═C1C═C(OC)C═C2)C(OCOC(C3CCN(CC3)C4═CN(C)C═CC4═O)═O)C |
| 492 | 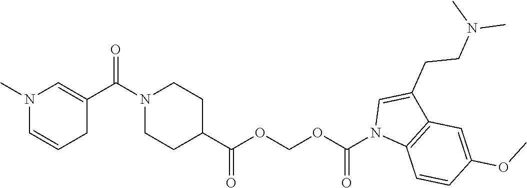 |
| | CN(CCC1═CN(C2═C1C═C(OC)C═C2)C(OCOC(C3CCN(CC3)C(C4═CN(C)C═CC4)═O)═O)═O)C |
| 493 | 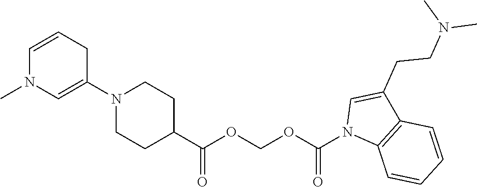 |
| | CN(CCC1═CN(C2═C1C═CC═C2)C(OCOC(C3CCN(CC3)C4═CN(C)C═CC4)═O)═O)C |
| 494 | 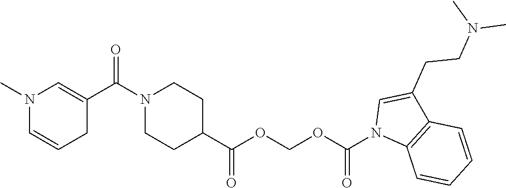 |
| | CN(CCC1═CN(C2═C1C═CC═C2)C(OCOC(C3CCN(CC3)C(C4═CN(C)C═CC4)═O)═O)═O)C |
| 495 | 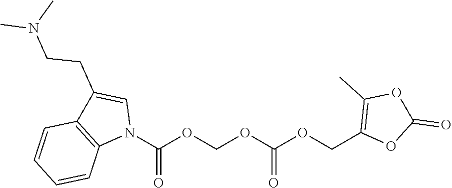 [SMILES: Cc1oc(=O)oc1COC(=O)OCOC(=O)n1cc(CCN(C)C)c2ccccc21] |
| | CN(CCC1═CN(C2═C1C═CC═C2)C(OCOC(OCC(OC3═O)═C(O3)C)═O)═O)C |
| 496 | 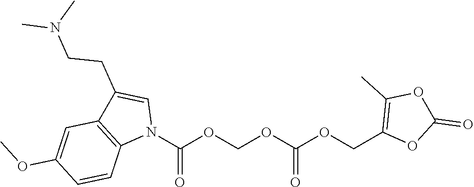 [SMILES: COc1ccc2c(c1)c(CCN(C)C)cn2C(=O)OCOC(=O)OCc1oc(=O)oc1C] |
| | CN(CCC1═CN(C2═C1C═C(OC)C═C2)C(OCOC(OCC(OC3═O)═C(O3)C)═O)═O)C |
| 497 | 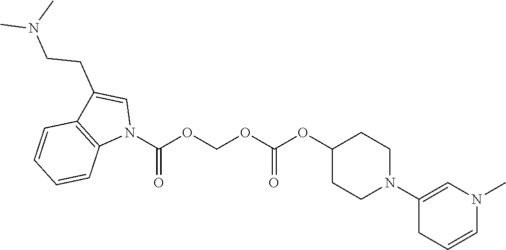 |
| | CN(CCC1═CN(C2═C1C═CC═C2)C(OCOC(OC3CCN(CC3)C4═CN(C)C═CC4)═O)═O)C |
| 498 | 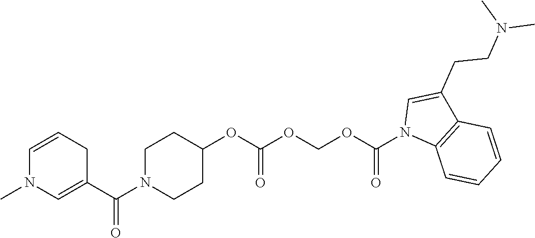 |
| | CN(CCC1═CN(C2═C1C═CC═C2)C(OCOC(OC3CCN(CC3)C(C4═CN(C)C═CC4)═O)═O)═O)C |
| 499 | 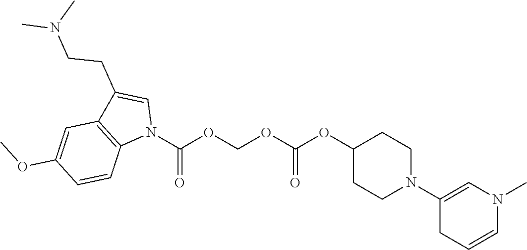 |
| | CN(CCC1═CN(C2═C1C═C(OC)C═C2)C(OCOC(OC3CCN(CC3)C4═CN(C)C═CC4)═O)═O)C |
| 500 | 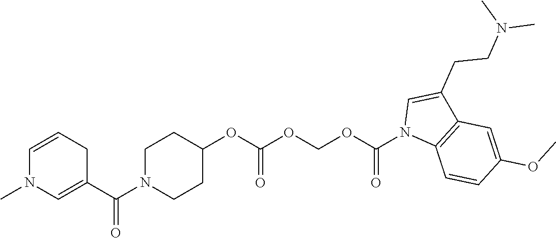 |
| | CN(CCC1═CN(C2═C1C═C(OC)C═C2)C(OCOC(OC3CCN(CC3)C(C4═CN(C)C═CC4)═O)═O)═O)C |
| 501 | 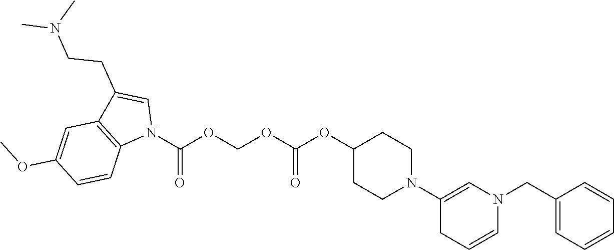 [SMILES: COc1ccc2c(c1)c(CCN(C)C)cn2C(=O)OCOC(=O)OC1CCN(C2=CN(Cc3ccccc3)C=CC2)CC1] |
| | CN(CCC1═CN(C2═C1C═C(OC)C═C2)C(OCOC(OC3CCN(CC3)C4═CN(C═CC4)CC5═CC═CC═C5)═O)═O)C |
| 502 | 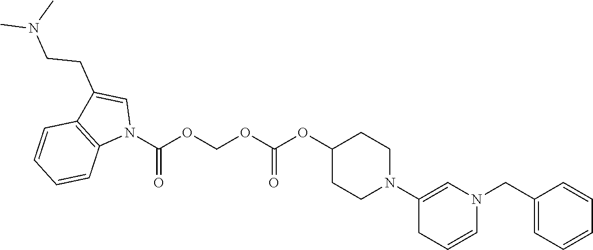 [SMILES: CN(C)CCc1cn(C(=O)OCOC(=O)OC2CCN(C3=CN(Cc4ccccc4)C=CC3)CC2)c2ccccc12] |
| | O═C(N(C1═C2C═CC═C1)C═C2CCN(C)C)OCOC(OC3CCN(CC3)C4═CN(C═CC4)CC5═CC═CC═C5)═O |
| 503 | 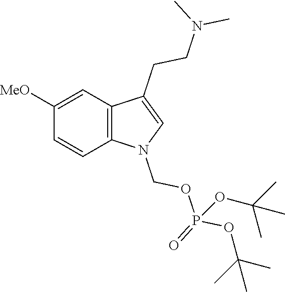 [SMILES: COc1ccc2c(c1)c(CCN(C)C)cn2COP(=O)(OC(C)(C)C)OC(C)(C)C] |
| | CN(C)CCC1═CN(COP(OC(C)(C)C)(OC(C)(C)C)═O)C2═C1C═C(OC)C═C2 |
| 504 | 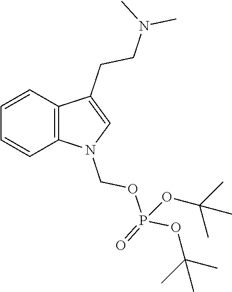 [SMILES: CN(C)CCc1cn(COP(=O)(OC(C)(C)C)OC(C)(C)C)c2ccccc12] |
| | CN(C)CCC1═CN(COP(OC(C)(C)C)(OC(C)(C)C)═O)C2═C1C═CC═C2 |
| 505 | 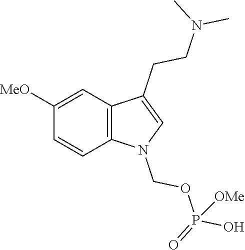 [SMILES: COc1ccc2c(c1)c(CCN(C)C)cn2COP(=O)(O)OC] |
| | CN(C)CCC1═CN(COP(O)(OC)═O)C2═C1C═C(OC)C═C2 |
| 506 | 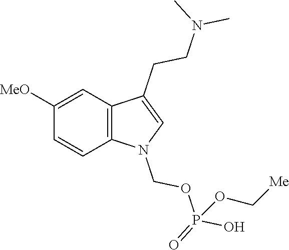 [SMILES: CCOP(=O)(O)OCn1cc(CCN(C)C)c2cc(OC)ccc21] |
| | CN(C)CCC1═CN(COP(O)(OCC)═O)C2═C1C═C(OC)C═C2 |
| 507 | 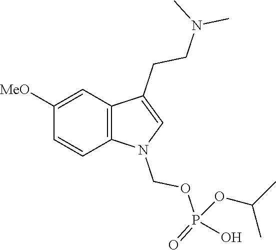 [SMILES: COc1ccc2c(c1)c(CCN(C)C)cn2COP(=O)(O)OC(C)C] |
| | CN(C)CCC1═CN(COP(O)(OC(C)C)═O)C2═C1C═C(OC)C═C2 |
| 508 | 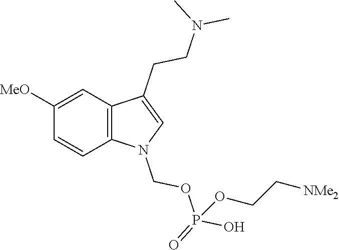 [SMILES: COc1ccc2c(c1)c(CCN(C)C)cn2COP(=O)(O)OCCN(C)C] |
| | CN(C)CCC1═CN(COP(O)(OCCN(C)C)═O)C2═C1C═C(OC)C═C2 |
| 509 | 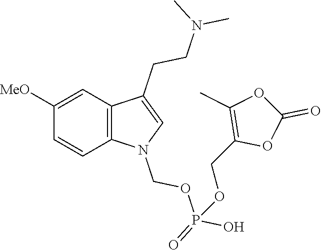 [SMILES: COc1ccc2c(c1)c(CCN(C)C)cn2COP(=O)(O)OCc1oc(=O)oc1C] |
| | CN(C)CCC1═CN(COP(O)(OCC(O2)═C(C)OC2═O)═O)C3═C1C═C(OC)C═C3 |
| 510 | 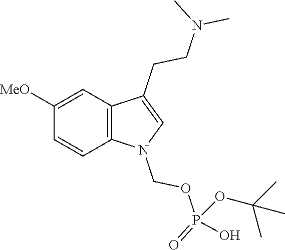 [SMILES: COc1ccc2c(c1)c(CCN(C)C)cn2COP(=O)(O)OC(C)(C)C] |
| | CN(C)CCC1═CN(COP(O)(OC(C)(C)C)═O)C2═C1C═C(OC)C═C2 |
| 511 | 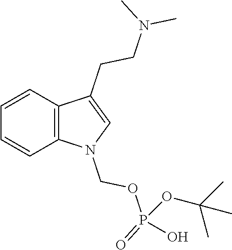 [SMILES: CN(C)CCc1cn(COP(=O)(O)OC(C)(C)C)c2ccccc12] |
| | CN(C)CCC1═CN(COP(O)(OC(C)(C)C)═O)C2═C1C═CC═C2 |
| 512 | 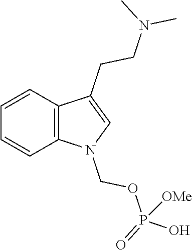 [SMILES: COP(=O)(O)OCn1cc(CCN(C)C)c2ccccc21] |
| | CN(C)CCC1═CN(COP(O)(OC)═O)C2═C1C═CC═C2 |
| 513 | 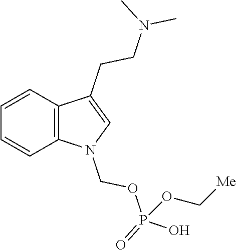 [SMILES: CCOP(=O)(O)OCn1cc(CCN(C)C)c2ccccc21] |
| | CN(C)CCC1═CN(COP(O)(OCC)═O)C2═C1C═CC═C2 |
| 514 | 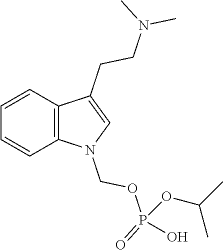 [SMILES: CC(C)OP(=O)(O)OCn1cc(CCN(C)C)c2ccccc21] |
| | CN(C)CCC1═CN(COP(O)(OC(C)C)═O)C2═C1C═CC═C2 |
| 515 | 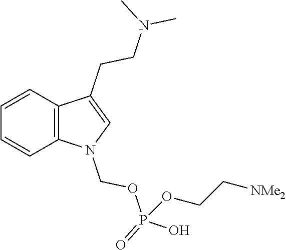 [SMILES: CN(C)CCOP(=O)(O)OCn1cc(CCN(C)C)c2ccccc21] |
| | CN(C)CCC1═CN(COP(O)(OCCN(C)C)═O)C2═C1C═CC═C2 |
| 516 | 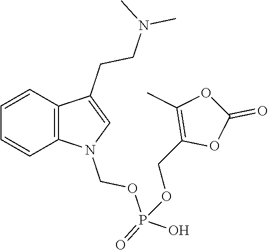 [SMILES: Cc1oc(=O)oc1COP(=O)(O)OCn1cc(CCN(C)C)c2ccccc21] |
| | CN(C)CCC1═CN(COP(O)(OCC(O2)═C(C)OC2═O)═O)C3═C1C═CC═C3 |
| 517 | 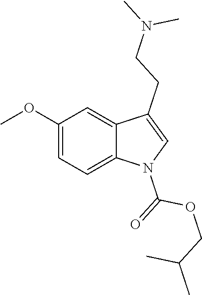 [SMILES: COc1ccc2c(c1)c(CCN(C)C)cn2C(=O)OCC(C)C] |
| | COC1═CC═C(N(C(OCC(C)C)═O)C═C2CCN(C)C)C2═C1 |
| 518 | 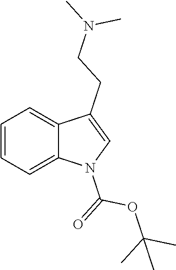 [SMILES: CN(C)CCc1cn(C(=O)OC(C)(C)C)c2ccccc12] |
| | CN(C)CCC1═CN(C(OC(C)(C)C)═O)C2═CC═CC═C21 |
| 519 | 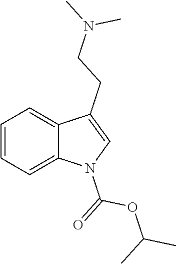 [SMILES: CC(C)OC(=O)n1cc(CCN(C)C)c2ccccc21] |
| | CN(C)CCC1═CN(C(OC(C)C)═O)C2═CC═CC═C21 |
| 520 | 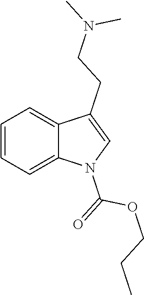 [SMILES: CCCOC(=O)n1cc(CCN(C)C)c2ccccc21] |
| | CN(C)CCC1═CN(C(OCCC)═O)C2═CC═CC═C21 |
| 521 | 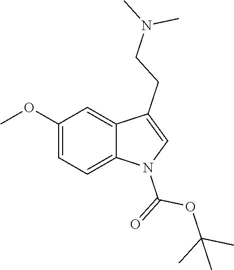 [SMILES: COc1ccc2c(c1)c(CCN(C)C)cn2C(=O)OC(C)(C)C] |
| | COC1═CC═C(N(C(OC(C)(C)C)═O)C═C2CCN(C)C)C2═C1 |
| 522 | 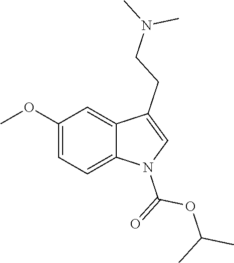 [SMILES: COc1ccc2c(c1)c(CCN(C)C)cn2C(=O)OC(C)C] |
| | COC1═CC═C(N(C(OC(C)C)═O)C═C2CCN(C)C)C2═C1 |
| 523 | 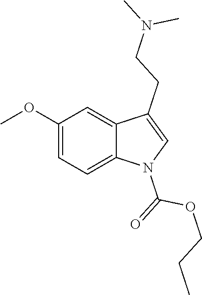 [SMILES: CCCOC(=O)n1cc(CCN(C)C)c2cc(OC)ccc21] |
| | COC1═CC═C(N(C(OCCC)═O)C═C2CCN(C)C)C2═C1 |
| 524 | 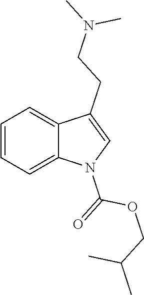 [SMILES: CC(C)COC(=O)n1cc(CCN(C)C)c2ccccc21] |
| | CN(C)CCC1═CN(C(OCC(C)C)═O)C2═CC═CC═C21 |
| 525 | 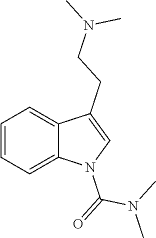 [SMILES: CN(C)CCc1cn(C(=O)N(C)C)c2ccccc12] |
| | CN(C)CCC1═CN(C(N(C)C)═O)C2═CC═CC═C21 |
| 526 | 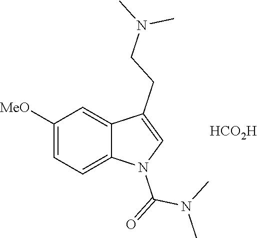 [SMILES: COc1ccc2c(c1)c(CCN(C)C)cn2C(=O)N(C)C ; "HCO2H"] |
| | CN(C)CCC1═CN(C(N(C)C)═O)C2═CC═C(OC)C═C21 |
| 529 | 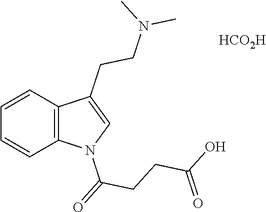 [SMILES: CN(C)CCc1cn(C(=O)CCC(=O)O)c2ccccc12 ; "HCO2H"] |
| | CN(C)CCC1═CN(C(CCC(O)═O)═O)C2═C1C═CC═C2 |
| 530 | 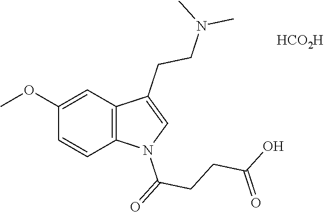 [SMILES: COc1ccc2c(c1)c(CCN(C)C)cn2C(=O)CCC(=O)O ; "HCO2H"] |
| | CN(C)CCC1═CN(C(CCC(O)═O)═O)C2═C1C═C(OC)C═C2 |
| 531 | 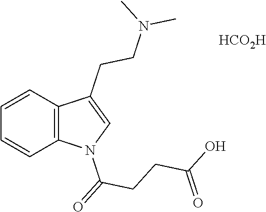 [SMILES: CN(C)CCc1cn(C(=O)CCC(=O)O)c2ccccc12 ; "HCO2H"] |
| | CN(C)CCC1═CN(C(CCC(O)═O)═O)C2═C1C═CC═C2 |
| 532 | 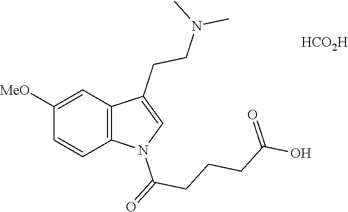 [SMILES: COc1ccc2c(c1)c(CCN(C)C)cn2C(=O)CCCC(=O)O ; "HCO2H"] |
| | CN(C)CCC1═CN(C(CCCC(O)═O)═O)C2═C1C═C(OC)C═C2 |
| 533 | 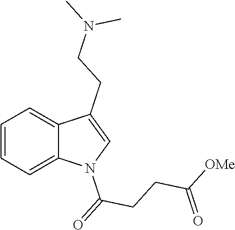 [SMILES: COC(=O)CCC(=O)n1cc(CCN(C)C)c2ccccc21] |
| | CN(C)CCC1═CN(C(CCC(OC)═O)═O)C2═CC═CC═C21 |
| 534 | 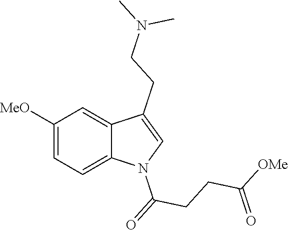 [SMILES: COC(=O)CCC(=O)n1cc(CCN(C)C)c2cc(OC)ccc21] |
| | CN(C)CCC1═CN(C(CCC(OC)═O)═O)C2═CC═C(OC)C═C21 |
| 535 | 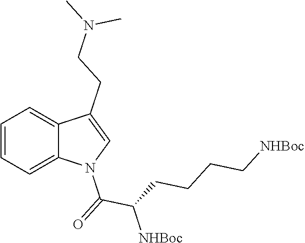 [SMILES: CN(C)CCc1cn(C(=O)[C@H](CCCCNC(=O)OC(C)(C)C)NC(=O)OC(C)(C)C)c2ccccc12] |
| | CN(C)CCC1═CN(C([C@@H](NC(OC(C)(C)C)═O)CCCCNC(OC(C)(C)C)═O)═O)C2═CC═CC═C21 |
| 536 | 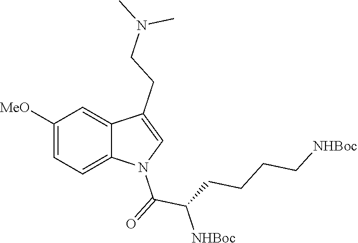 [SMILES: COc1ccc2c(c1)c(CCN(C)C)cn2C(=O)[C@H](CCCCNC(=O)OC(C)(C)C)NC(=O)OC(C)(C)C] |
| | CN(C)CCC1═CN(C([C@@H](NC(OC(C)(C)C)═O)CCCCNC(OC(C)(C)C)═O)═O)C2═CC═C(OC)C═C21 |
| 537 | 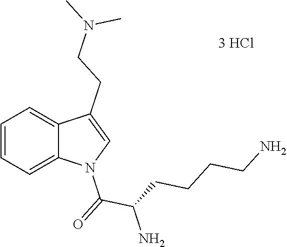 [SMILES: CN(C)CCc1cn(C(=O)[C@@H](N)CCCCN)c2ccccc12] |
| | C(C)CCC1═CN(C([C@@H](N)CCCCN)═O)C2═CC═CC═C21 |
| 538 | 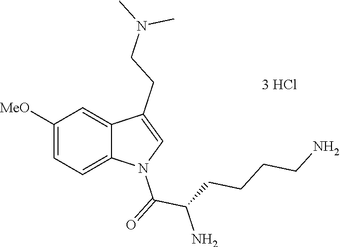 [SMILES: COc1ccc2c(c1)c(CCN(C)C)cn2C(=O)[C@@H](N)CCCCN] |
| | CN(C)CCC1═CN(C([C@@H](N)CCCCN)═O)C2═CC═C(OC)C═C21 |
| 539 | 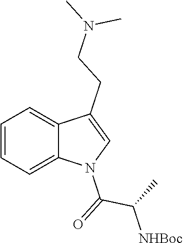 [SMILES: C[C@H](NC(=O)OC(C)(C)C)C(=O)n1cc(CCN(C)C)c2ccccc21] |
| | CN(C)CCC1═CN(C([C@@H](NC(OC(C)(C)C)═O)C)═O)C2═CC═CC═C21 |
| 540 | 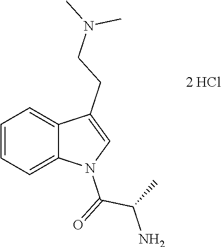 [SMILES: C[C@H](N)C(=O)n1cc(CCN(C)C)c2ccccc21] |
| | CN(C)CCC1═CN(C([C@@H](N)C)═O)C2═CC═CC═C21 |
| 541 | 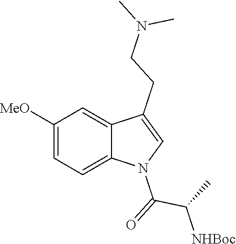 [SMILES: COc1ccc2c(c1)c(CCN(C)C)cn2C(=O)[C@H](C)NC(=O)OC(C)(C)C] |
| | CN(C)CCC1═CN(C([C@@H](NC(OC(C)(C)C)═O)C)═O)C2═CC═C(OC)C═C21 |
| 542 | 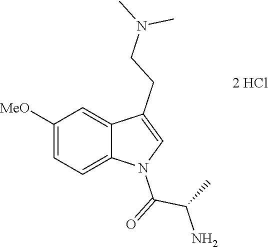 [SMILES: COc1ccc2c(c1)c(CCN(C)C)cn2C(=O)[C@H](C)N] |
| | CN(C)CCC1═CN(C([C@@H](N)C)═O)C2═CC═C(OC)C═C21 |
| 543 | 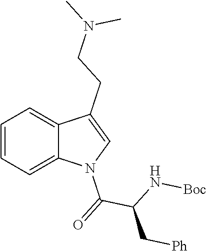 [SMILES: CN(C)CCc1cn(C(=O)[C@H](Cc2ccccc2)NC(=O)OC(C)(C)C)c2ccccc12] |
| | CN(C)CCC1═CN(C([C@H](CC2═CC═CC═C2)NC(OC(C)(C)C)═O)═O)C3═CC═CC═C31 |
| 544 | 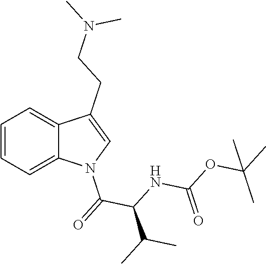 [SMILES: CC(C)[C@H](NC(=O)OC(C)(C)C)C(=O)n1cc(CCN(C)C)c2ccccc21] |
| | CN(C)CCC1═CN(C([C@H](C(C)C)NC(OC(C)(C)C)═O)═O)C2═CC═CC═C21 |
| 545 | 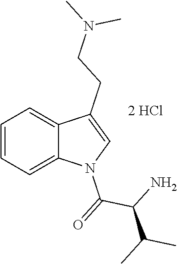 [SMILES: CC(C)[C@H](N)C(=O)n1cc(CCN(C)C)c2ccccc21] |
| | CN(C)CCC1═CN(C([C@H](C(C)C)N)═O)C2═CC═CC═C21 |
| 546 | 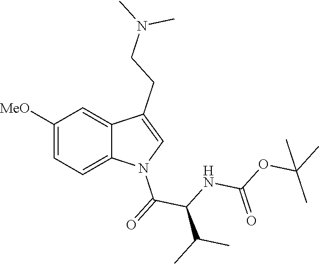 [SMILES: COc1ccc2c(c1)c(CCN(C)C)cn2C(=O)[C@@H](NC(=O)OC(C)(C)C)C(C)C] |
| | CN(C)CCC1═CN(C([C@H](C(C)C)NC(OC(C)(C)C)═O)═O)C2═CC═C(OC)C═C21 |
| 547 | 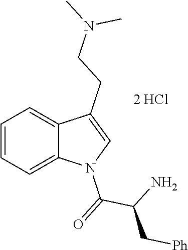 [SMILES: CN(C)CCc1cn(C(=O)[C@@H](N)Cc2ccccc2)c2ccccc12] |
| | CN(C)CCC1═CN(C([C@H](CC2═CC═CC═C2)N)═O)C3═CC═CC═C31 |
| 548 | 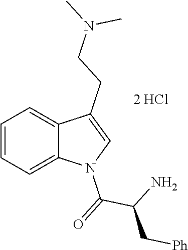 [SMILES: CN(C)CCc1cn(C(=O)[C@@H](N)Cc2ccccc2)c2ccccc12] |
| | CN(C)CCC1═CN(C([C@H](CC2═CC═CC═C2)N)═O)C3═CC═CC═C31 |
| 549 | 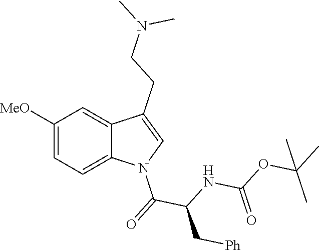 [SMILES: COc1ccc2c(c1)c(CCN(C)C)cn2C(=O)[C@H](Cc1ccccc1)NC(=O)OC(C)(C)C] |
| | CN(C)CCC1═CN(C([C@H](CC2═CC═CC═C2)NC(OC(C)(C)C)═O)═O)C3═CC═C(OC)C═C31 |
| 550 | 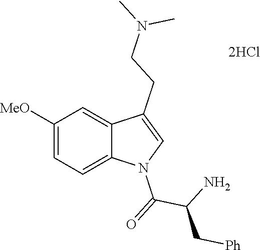 [SMILES: COc1ccc2c(c1)c(CCN(C)C)cn2C(=O)[C@@H](N)Cc1ccccc1] |
| | CN(C)CCC1═CN(C([C@H](CC2═CC═CC═C2)N)═O)C3═CC═C(OC)C═C31 |
| 551 | 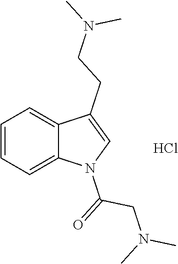 [SMILES: CN(C)CCc1cn(C(=O)CN(C)C)c2ccccc12 ; Cl] |
| | CN(C)CCC1═CN(C(CN(C)C)═O)C2═CC═CC═C21 |
| 552 | 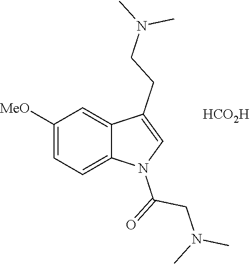 [SMILES: COc1ccc2c(c1)c(CCN(C)C)cn2C(=O)CN(C)C ; "HCO2H"] |
| | CN(C)CCC1═CN(C(CN(C)C)═O)C2═CC═C(OC)C═C21 |
| 553 | 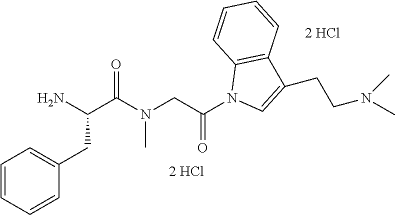 [SMILES: CN(C)CCc1cn(C(=O)CN(C)C(=O)[C@@H](N)Cc2ccccc2)c2ccccc12] |
| | O═C(N(C)CC(N1C═C(CCN(C)C)C2═C1C═CC═C2)═O)[C@H](CC3═CC═CC═C3)N |
| 554 | 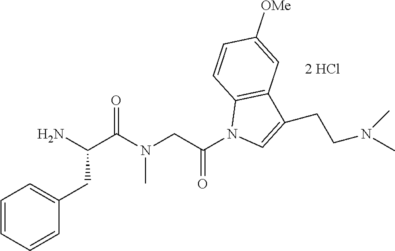 [SMILES: COc1ccc2c(c1)c(CCN(C)C)cn2C(=O)CN(C)C(=O)[C@@H](N)Cc1ccccc1] |
| | O═C(N(C)CC(N1C═C(CCN(C)C)C2═C1C═CC(OC)═C2)═O)[C@H](CC3═CC═CC═C3)N |
| 555 | 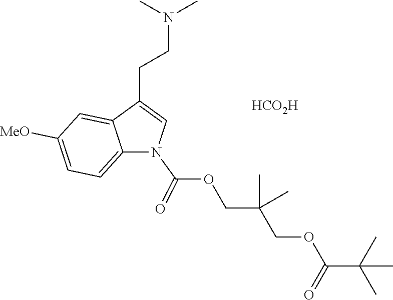 [SMILES: COc1ccc2c(c1)c(CCN(C)C)cn2C(=O)OCC(C)(C)COC(=O)C(C)(C)C ; "HCO2H"] |
| | CN(C)CCC1═CN(C(OCC(C)(C)COC(C(C)(C)C)═O)═O)C2═CC═C(OC)C═C21 |
| 556 | 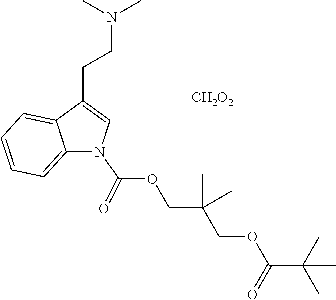 [SMILES: CN(C)CCc1cn(C(=O)OCC(C)(C)COC(=O)C(C)(C)C)c2ccccc12 ; "CH2O2"] |
| | CN(C)CCC1═CN(C(OCC(C)(C)COC(C(C)(C)C)═O)═O)C2═CC═CC═C21 |
| 557 | 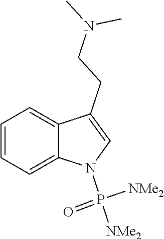 [SMILES: CN(C)CCc1cn(P(=O)(N(C)C)N(C)C)c2ccccc12] |
| | CN(C)CCC1═CN(P(N(C)C)(N(C)C)═O)C2═CC═CC═C21 |
| 558 | 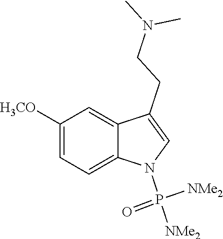 [SMILES: COc1ccc2c(c1)c(CCN(C)C)cn2P(=O)(N(C)C)N(C)C] |
| | CN(C)CCC1═CN(P(N(C)C)(N(C)C)═O)C2═CC═C(OC)C═C21 |
| 559 | 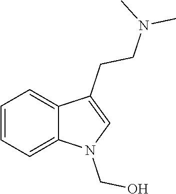 [SMILES: CN(C)CCc1cn(CO)c2ccccc12] |
| | CN(C)CCC1═CN(CO)C2═CC═CC═C21 |
| 560 | 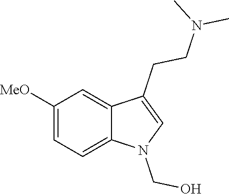 [SMILES: COc1ccc2c(c1)c(CCN(C)C)cn2CO] |
| | CN(C)CCC1═CN(CO)C2═CC═C(OC)C═C21 |
| 561 | 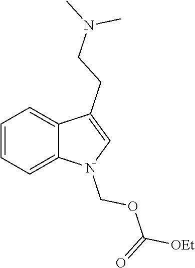 [SMILES: CCOC(=O)OCn1cc(CCN(C)C)c2ccccc21] |
| | CN(C)CCC1═CN(COC(OCC)═O)C2═CC═CC═C21 |
| 562 | 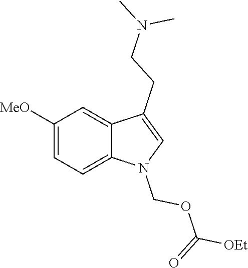 [SMILES: CCOC(=O)OCn1cc(CCN(C)C)c2cc(OC)ccc21] |
| | CN(C)CCC1═CN(COC(OCC)═O)C2═CC═C(OC)C═C21 |
| 563 | 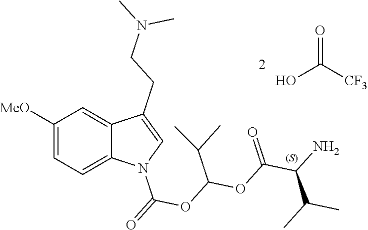 |
| | O═C(OC(C(C)C)OC([C@H](C(C)C)N)═O)N1C2═CC═C(OC)C═C2C(CCN(C)C)═C1 |
| 564 | 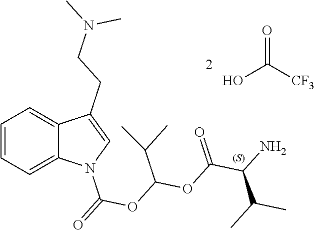 |
| | O═C(OC(C(C)C)OC([C@H](C(C)C)N)═O)N1C2═CC═CC═C2C(CCN(C)C)═C1 |
| 565 | 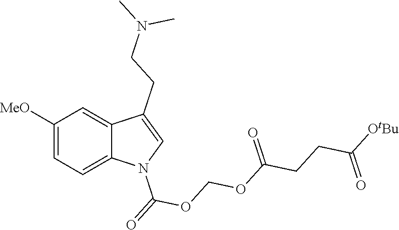 [SMILES: COc1ccc2c(c1)c(CCN(C)C)cn2C(=O)OCOC(=O)CCC(=O)OC(C)(C)C] |
| | CN(C)CCC1═CN(C(OCOC(CCC(OC(C)(C)C)═O)═O)═O)C2═CC═C(OC)C═C21 |
| 566 | 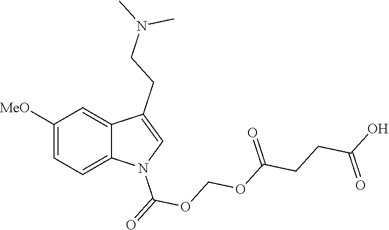 [SMILES: COc1ccc2c(c1)c(CCN(C)C)cn2C(=O)OCOC(=O)CCC(=O)O] |
| | CN(C)CCC1═CN(C(OCOC(CCC(O)═O)═O)═O)C2═CC═C(OC)C═C21 |
| 567 | 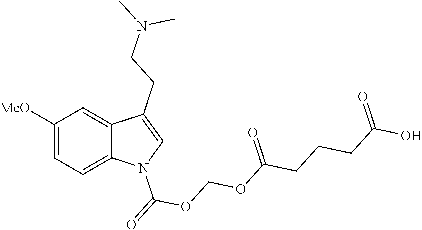 [SMILES: COc1ccc2c(c1)c(CCN(C)C)cn2C(=O)OCOC(=O)CCCC(=O)O] |
| | CN(C)CCC1═CN(C(OCOC(CCCC(O)═O)═O)═O)C2═CC═C(OC)C═C21 |
| 568 | 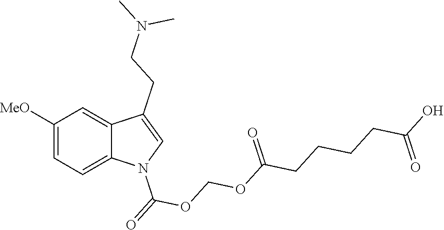 [SMILES: COc1ccc2c(c1)c(CCN(C)C)cn2C(=O)OCOC(=O)CCCCC(=O)O] |
| | CN(C)CCC1═CN(C(OCOC(CCCCC(O)═O)═O)═O)C2═CC═C(OC)C═C21 |
| 569 | 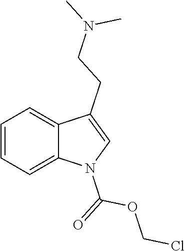 [SMILES: CN(C)CCc1cn(C(=O)OCCl)c2ccccc12] |
| | CN(C)CCC1═CN(C(OCCl)═O)C2═CC═CC═C21 |
| 570 | 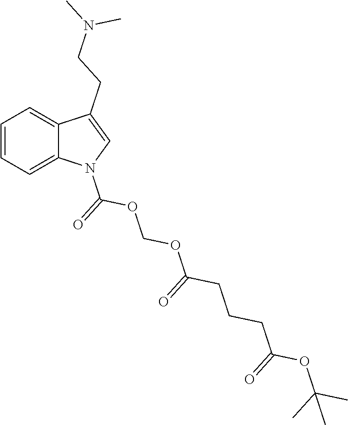 [SMILES: CN(C)CCc1cn(C(=O)OCOC(=O)CCCC(=O)OC(C)(C)C)c2ccccc12] |
| | CN(C)CCC1═CN(C(OCOC(CCCC(OC(C)(C)C)═O)═O)═O)C2═CC═CC═C21 |
| 571 | 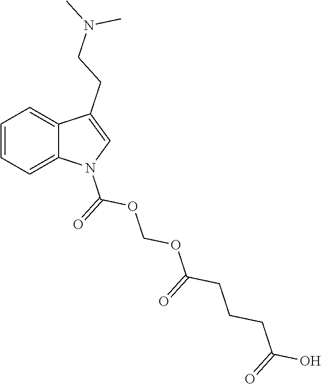 [SMILES: CN(C)CCc1cn(C(=O)OCOC(=O)CCCC(=O)O)c2ccccc12] |
| | CN(C)CCC1═CN(C(OCOC(CCCC(O)═O)═O)═O)C2═CC═CC═C21 |
| 572 | 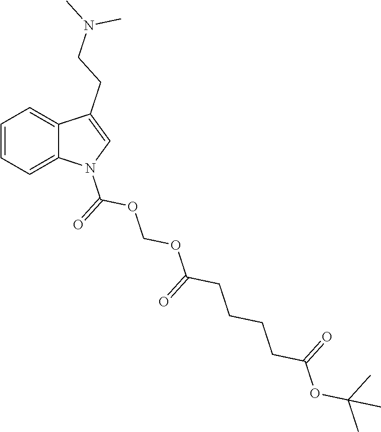 [SMILES: CN(C)CCc1cn(C(=O)OCOC(=O)CCCCC(=O)OC(C)(C)C)c2ccccc12] |
| | CN(C)CCC1═CN(C(OCOC(CCCCC(OC(C)(C)C)═O)═O)═O)C2═CC═CC═C21 |
| 573 | 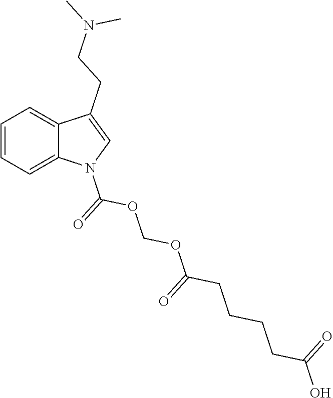 [SMILES: CN(C)CCc1cn(C(=O)OCOC(=O)CCCCC(=O)O)c2ccccc12] |
| | CN(C)CCC1═CN(C(OCOC(CCCCC(O)═O)═O)═O)C2═CC═CC═C21 |
| 574 | 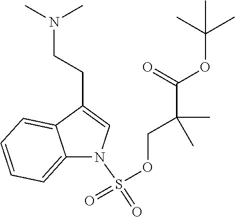 [SMILES: CN(C)CCc1cn(S(=O)(=O)OCC(C)(C)C(=O)OC(C)(C)C)c2ccccc12] |
| | CN(C)CCC1═CN(S(═O)(OCC(C)(C)C(OC(C)(C)C)═O)═O)C2═CC═CC═C21 |
| 575 | 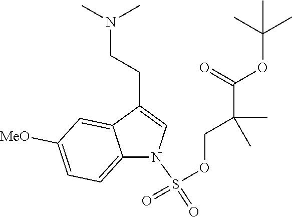 [SMILES: COc1ccc2c(c1)c(CCN(C)C)cn2S(=O)(=O)OCC(C)(C)C(=O)OC(C)(C)C] |
| | CN(C)CCC1═CN(S(═O)(OCC(C)(C)C(OC(C)(C)C)═O)═O)C2═CC═C(OC)C═C21 |
| 576 | 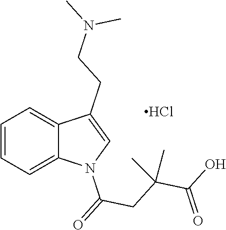 [SMILES: CN(C)CCc1cn(C(=O)CC(C)(C)C(=O)O)c2ccccc12 ; Cl] |
| | CN(C)CCC1═CN(C(CC(C)(C(O)═O)C)═O)C2═CC═CC═C21 |
| 577 | 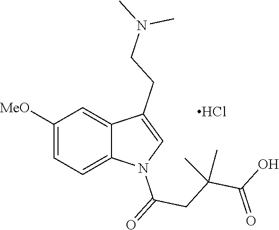 [SMILES: COc1ccc2c(c1)c(CCN(C)C)cn2C(=O)CC(C)(C)C(=O)O ; Cl] |
| | CN(C)CCC1═CN(C(CC(C)(C(O)═O)C)═O)C2═CC═C(OC)C═C21 |

*SMILES strings of the corresponding freebase are provided for all compounds that are salts.

In some embodiments, the compound described herein is a compound selected from Table 1.

In some embodiment, the compound described herein a compound selected from Table 1A below.

TABLE 1A

| Com- | | |
|---|---|---|
| pound | Structure | Chemical Name |
| 20 | 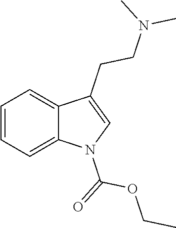 [SMILES: CCOC(=O)n1cc(CCN(C)C)c2ccccc21] | Ethyl 3-[2-(dimethylamino)- ethyl]indole-1-carboxylate |
| 19 | 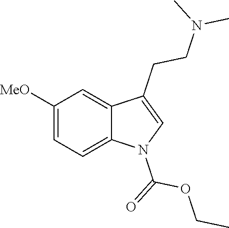 [SMILES: CCOC(=O)n1cc(CCN(C)C)c2cc(OC)ccc21] | Ethyl 3-[2-(dimethylamino)- ethyl]-5-methoxy-indole-1- carboxylate |
| 263 | 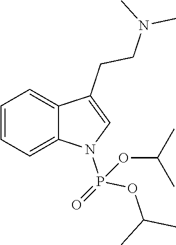 [SMILES: CC(C)OP(=O)(OC(C)C)n1cc(CCN(C)C)c2ccccc21] | 2-(1-Diisopropoxyphosphor- ylindol-3-yl)-N,N-dimethyl- ethanamine |
| 255 | 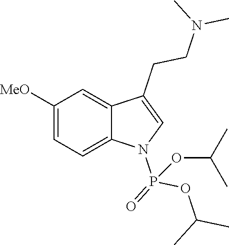 [SMILES: COc1ccc2c(c1)c(CCN(C)C)cn2P(=O)(OC(C)C)OC(C)C] | 2-(1-Diisopropoxyphosphor- yl-5-methoxy-indol-3-yl)- N,N-dimethyl-ethanamine |
| 511 | 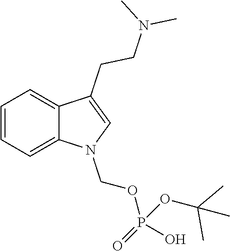 [SMILES: CN(C)CCc1cn(COP(=O)(O)OC(C)(C)C)c2ccccc12] | Tert-butyl [3-[2-(dimethyl- amino)ethyl]indol-1-yl]- methyl hydrogen |
| 510 | 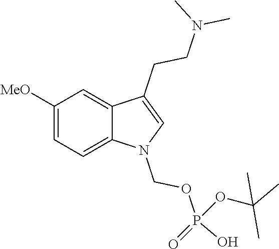 [SMILES: COc1ccc2c(c1)c(CCN(C)C)cn2COP(=O)(O)OC(C)(C)C] | Tert-butyl [3-[2-(dimethyl- amino)ethyl]-5-methoxy- indol-1-yl]methyl hydrogen phosphate |
| 517 | 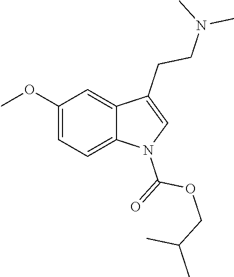 | Isobutyl 3-[2-(dimethyl- amino)ethyl]-6-methoxy- indole-1-carboxylate |
| 518 | 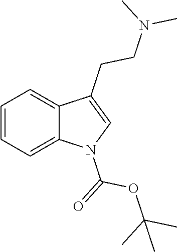 [SMILES: CN(C)CCc1cn(C(=O)OC(C)(C)C)c2ccccc12] | tert-butyl 3-[2-(dimethyl- amino)ethyl]indole-1- carboxylate |
| 519 | 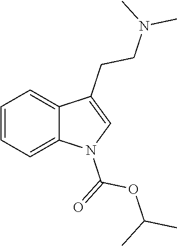 [SMILES: CC(C)OC(=O)n1cc(CCN(C)C)c2ccccc21] | isopropyl 3-[2-(dimethyl- amino)ethyl]indole-1- carboxylate |
| 520 | 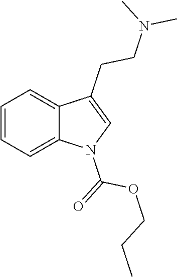 [SMILES: CCCOC(=O)n1cc(CCN(C)C)c2ccccc21] | propyl 3-[2-(dimethylamino)- ethyl]indole-1-carboxylate |
| 521 | 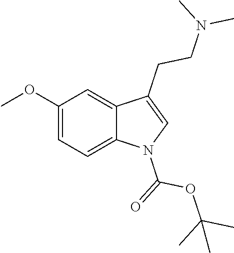 | tert-butyl 3-[2-(dimethyl- amino)ethyl]-5-methoxy- indole-1-carboxylate |
| 522 | 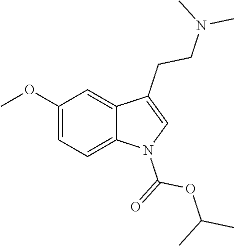 | isopropyl 3-[2-(dimethyl- amino)ethyl]-5-methoxy- indole-1-carboxylate |
| 523 | 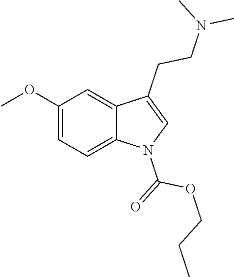 | propyl 3-[2-(dimethylamino)- ethyl]-5-methoxy-indole-1- carboxylate |
| 524 | 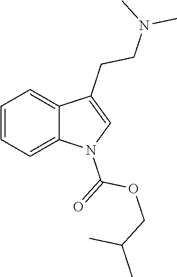 [SMILES: CC(C)COC(=O)n1cc(CCN(C)C)c2ccccc21] | isobutyl 3-[2-(dimethyl- amino)ethyl]indole-1- carboxylate^{a} |
| 119 | 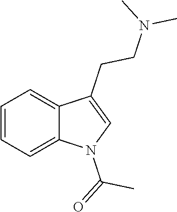 [SMILES: CC(=O)n1cc(CCN(C)C)c2ccccc21] | 1-[3-[2-(dimethylamino)- ethyl]indol-1-yl]ethenone |
| 122 | 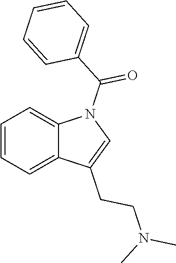 [SMILES: CN(C)CCc1cn(C(=O)c2ccccc2)c2ccccc12] | [3-[2-(dimethylamino)ethyl]- indol-1-yl]-phenyl-methanone |
| 120 | 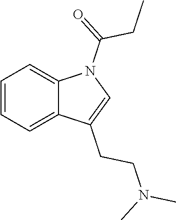 [SMILES: CCC(=O)n1cc(CCN(C)C)c2ccccc21] | 1-[3-[2-(dimethylamino)- ethyl]indol-1-yl]propan-1- one |
| 108 | 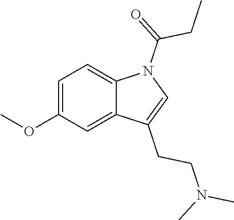 | 1-[3-[2-(dimethylamino)- ethyl]-5-methoxy-indol-1- yl]propan-1-one |
| 110 | 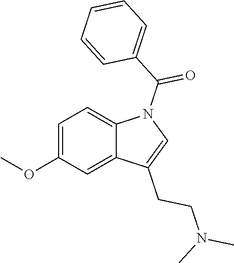 | [3-[2-(dimethylamino)ethyl]- 5-methoxy-indol-1-yl]-phenyl- methanone |
| 107 | 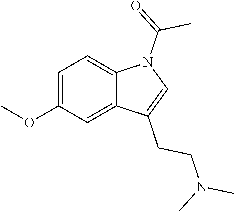 | 1-[3-[2-(dimethylamino)ethyl]- 5-methoxy-indol-1-yl]ethanone |
| 525 | 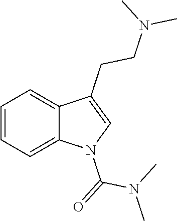 [SMILES: CN(C)CCc1cn(C(=O)N(C)C)c2ccccc12] | 3-[2-(dimethylamino)ethyl]- N,N-dimethyl-indole-1- carboxamide |
| 526 | 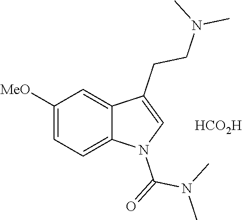 [SMILES: COc1ccc2c(c1)c(CCN(C)C)cn2C(=O)N(C)C ; "HCO2H"] | 3-(2-(dimethylamino)ethyl)- 5-methoxy-N,N-dimethyl- 1H-indole-1-carboxamide formate |
| 88 | 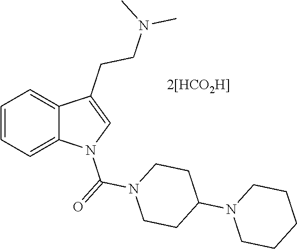 [SMILES: CN(C)CCc1cn(C(=O)N2CCC(N3CCCCC3)CC2)c2ccccc12] | [1,4′-Bipiperidin]-1′-yl(3-(2- (dimethylamino)ethyl)-1H- indol-1-yl)methanone di- formate |
| 96 | 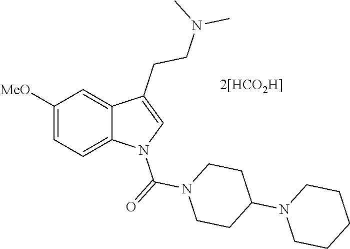 [SMILES: COc1ccc2c(c1)c(CCN(C)C)cn2C(=O)N1CCC(N2CCCCC2)CC1] | [1,4′-bipiperidin]-1′-yl(3-(2- (dimethylamino)ethyl)-5- methoxy-1H-indol-1-yl)- methanone di-formate |
| 413 | 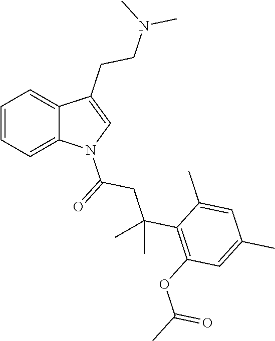 [SMILES: CC(=O)Oc1cc(C)cc(C)c1C(C)(C)CC(=O)n1cc(CCN(C)C)c2ccccc21] | 2-(4-(3-(2-(dimethylamino)- ethyl)-1H-indol-1-yl)-2- methyl-4-oxobutan-2-yl)-3,5- dimethylphenyl acetate |
| 405 | 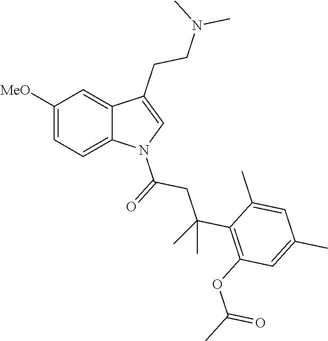 [SMILES: COc1ccc2c(c1)c(CCN(C)C)cn2C(=O)CC(C)(C)c1c(C)cc(C)cc1OC(C)=O] | 2-(4-(3-(2-(dimethylamino)- ethyl)-5-methoxy-1H-indol-1- yl)-2-methyl-4-oxobutan-2- yl)-3,5-dimethylphenyl |
| 25 | 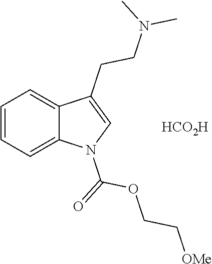 [SMILES: COCCOC(=O)n1cc(CCN(C)C)c2ccccc21 ; "HCO2H"] | 2-Methoxyethyl 3-(2- (dimethylamino)ethyl)-1H- indole-1-carboxylate formate |
| 22 | 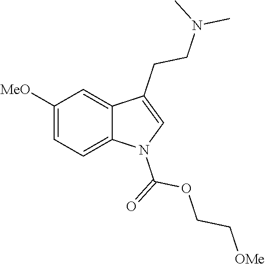 [SMILES: COCCOC(=O)n1cc(CCN(C)C)c2cc(OC)ccc21] | 2-Methoxyethyl 3-(2- (dimethylamino)ethyl)-5- methoxy-1H-indole-1-carbox- ylate formate |
| 529 | 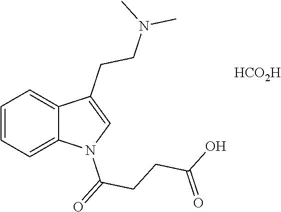 [SMILES: CN(C)CCc1cn(C(=O)CCC(=O)O)c2ccccc12 ; "HCO2H"] | 4-(3-(2-(dimethylamino)- ethyl)-1H-indol-1-yl)-4-oxo- butanoic acid formate salt |
| 530 | 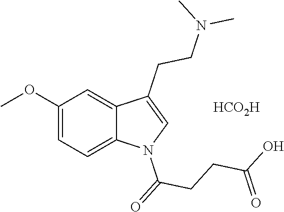 [SMILES: COc1ccc2c(c1)c(CCN(C)C)cn2C(=O)CCC(=O)O ; "HCO2H"] | 4-(3-(2-(dimethylamino)- ethyl)-5-methoxy-1H-indol- 1-yl)-4-oxobutanoic acid formate salt |
| 531 | 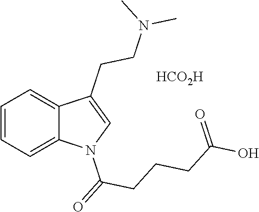 [SMILES: CN(C)CCc1cn(C(=O)CCCC(=O)O)c2ccccc12 ; "HCO2H"] | 5-(3-(2-(dimethylamino)- ethyl)-1H-indol-1-yl)-5-oxo- pentanoic acid formate salt |
| 532 | 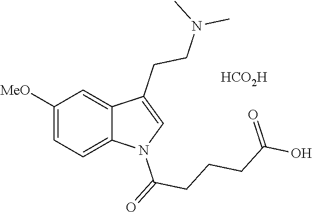 [SMILES: COc1ccc2c(c1)c(CCN(C)C)cn2C(=O)CCCC(=O)O ; "HCO2H"] | 5-(3-(2-(dimethylamino)- ethyl)-5-methoxy-1H-indol- 1-yl)-5-oxopentanoic acid formate salt |
| 369 | 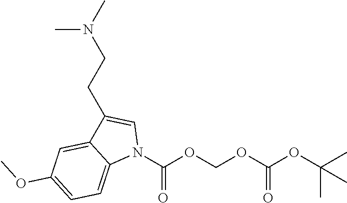 [SMILES: COc1ccc2c(c1)c(CCN(C)C)cn2C(=O)OCOC(=O)OC(C)(C)C] | (Pivaloyloxy)methyl 3-(2- (dimethylamino)ethyl)-5- methoxy-1H-indole-1- carboxylate |
| 387 | 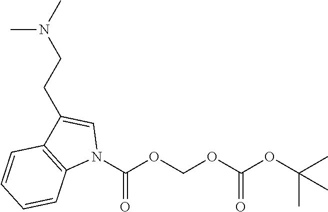 [SMILES: CN(C)CCc1cn(C(=O)OCOC(=O)OC(C)(C)C)c2ccccc12] | (Pivaloyloxy)methyl 3-(2- (dimethylamino)ethyl)-1H- indole-1-carboxylate diformat |
| 533 | 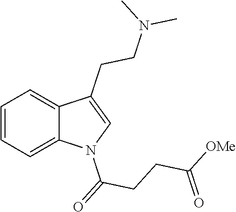 [SMILES: COC(=O)CCC(=O)n1cc(CCN(C)C)c2ccccc21] | Methyl 4-(3-(2-(dimethyl- amino)ethyl)-1H-indol-1-yl)- 4-oxobutanoate |
| 534 | 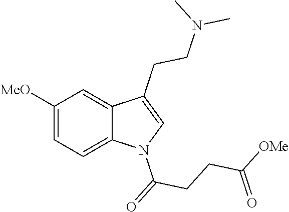 [SMILES: COC(=O)CCC(=O)n1cc(CCN(C)C)c2cc(OC)ccc21] | Methyl 4-(3-(2-(dimethyl- amino)ethyl)-5-methoxy-1H- indol-1-yl)-4-oxobutanoate |
| 535 | 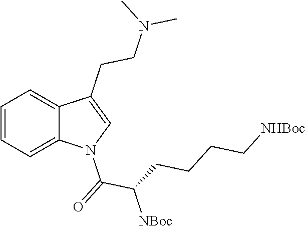 | (S)-di-tert-butyl (6-(3-(2- (dimethylamino)ethyl)-1H- indol-1-yl)-6-oxohexane-1,5- diyl)dicarbamate |
| 536 | 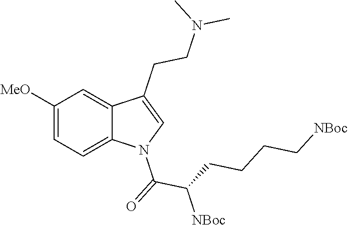 | (S)-di-tert-butyl (6-(3-(2- (dimethylamino)ethyl)-5- methoxy-1H-indol-1-yl)-6- oxohexane-1,5-diyl)- dicarbamate |
| 537 | 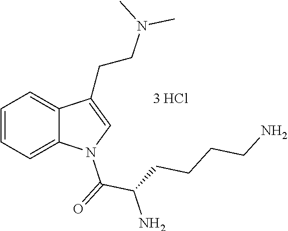 [SMILES: CN(C)CCc1cn(C(=O)[C@@H](N)CCCCN)c2ccccc12] | (S)-2,6-diamino-1-(3-(2- (dimethylamino)ethyl-1H- indol-1-yl)hexan-1-one trihydrochloride |
| 538 | 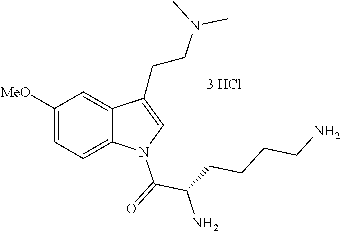 [SMILES: COc1ccc2c(c1)c(CCN(C)C)cn2C(=O)[C@@H](N)CCCCN] | (S)-2,6-diamino-1-(3-(2- (dimethylamino)ethyl)-5- methoxy-1H-indol-1-yl)- hexan-1-one trihydrochloride |
| 539 | 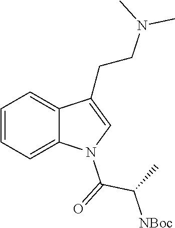 | (S)-tert-butyl (1-(3-(2- (dimethylamino)ethyl)-1H- indol-1-yl)-1-oxopropan-2- yl)carbamate |
| 540 | 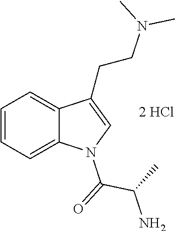 [SMILES: C[C@H](N)C(=O)n1cc(CCN(C)C)c2ccccc21] | (S)-2-amino-1-(3-(2-(dimeth- ylamino)ethyl)-1H-indol-1- yl)propan-l-one dihydro- chloride |
| 541 | 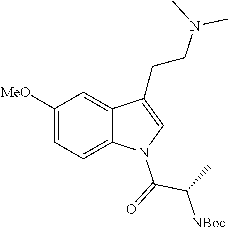 | (S)-tert-butyl (1-(3-(2- (dimethylamino)ethyl)-5- methoxy-1H-indol-1-yl)-1- oxopropan-2-yl)carbamate |
| 542 | 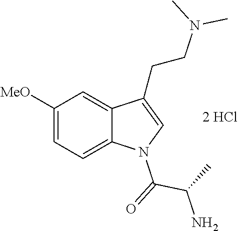 [SMILES: COc1ccc2c(c1)c(CCN(C)C)cn2C(=O)[C@H](C)N] | (S)-2-amino-1-(3-(2-(dimeth- ylamino)ethyl)-5-methoxy- 1H-indol-1-yl)propan-1-one dihydrochloride |
| 543 | 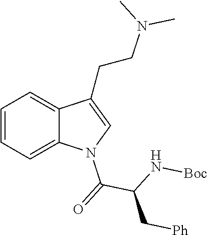 [SMILES: CN(C)CCc1cn(C(=O)[C@H](Cc2ccccc2)NC(=O)OC(C)(C)C)c2ccccc12] | (S)-tert-butyl (1-(3-(2- (dimethylamino)ethyl)-1H- indol-1-yl)-1-oxo-3-phenyl- propan-2-yl)carbamate |
| 544 | 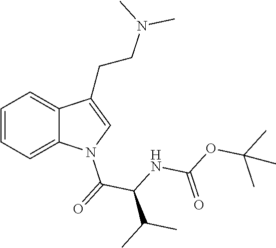 [SMILES: CC(C)[C@H](NC(=O)OC(C)(C)C)C(=O)n1cc(CCN(C)C)c2ccccc21] | (S)-tert-butyl (1-(3-(2- (dimethylamino)ethyl)-1H- indol-1-yl)-3-methyl-1-oxo- butan-2-yl)carbamate |
| 545 | 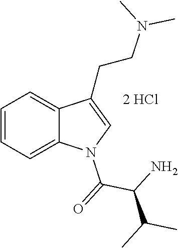 [SMILES: CC(C)[C@H](N)C(=O)n1cc(CCN(C)C)c2ccccc21] | (S)-2-amino-1-(3-(2-(dimeth- ylamino)ethyl)-1H-indol-1- yl)-3-methylbutan-1-one dihydrochloride |
| 546 | 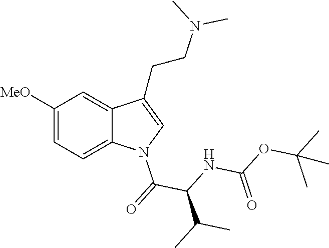 [SMILES: COc1ccc2c(c1)c(CCN(C)C)cn2C(=O)[C@@H](NC(=O)OC(C)(C)C)C(C)C] | (S)-tert-butyl (1-(3-(2- (dimethylamino)ethyl)-5- methoxy-1H-indol-1-yl)-3- methyl-1-oxobutan-2-yl)- carbamate |
| 547 | 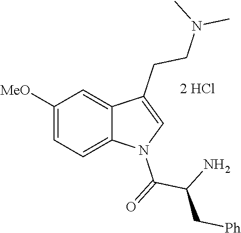 [SMILES: COc1ccc2c(c1)c(CCN(C)C)cn2C(=O)[C@@H](N)Cc1ccccc1] | (S)-2-amino-1-(3-(2-(dimeth- ylamino)ethyl)-5-methoxy- 1H-indol-1-yl)-3-methyl- butan-1-one bis-hydrochloride |
| 548 | 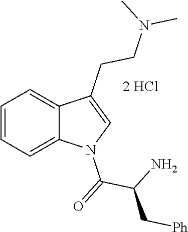 [SMILES: CN(C)CCc1cn(C(=O)[C@@H](N)Cc2ccccc2)c2ccccc12] | (S)-2-amino-1-(3-(2-(dimeth- ylamino)ethyl)-1H-indol-1- yl)-3-phenylpropan-1-one bis-hydrochloride |
| 549 | 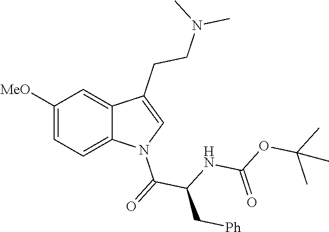 [SMILES: COc1ccc2c(c1)c(CCN(C)C)cn2C(=O)[C@H](Cc1ccccc1)NC(=O)OC(C)(C)C] | (S)-tert-butyl (1-(3-(2- (dimethylamino)ethyl)-5- methoxy-1H-indol-1-yl)-1- oxo-3-phenylpropan-2-yl)- carbamate |
| 550 | 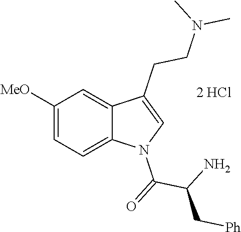 [SMILES: COc1ccc2c(c1)c(CCN(C)C)cn2C(=O)[C@@H](N)Cc1ccccc1] | (S)-2-amino-1-(3-(2-(dimeth- ylamino)ethyl)-5-methoxy- 1H-indol-1-yl)-3-phenyl- propan-1-one bis-hydro- chloride |
| 551 | 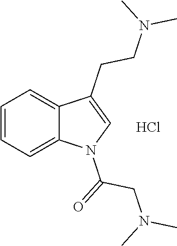 [SMILES: CN(C)CCc1cn(C(=O)CN(C)C)c2ccccc12 ; Cl] | 2-(Dimethylamino)-1-(3-(2- (dimethylamino)ethyl)-1H- indol-1-yl)ethan-1-one hydro- chloride |
| 552 | 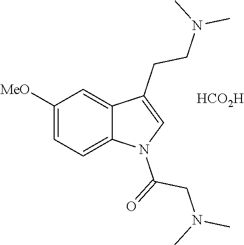 [SMILES: COc1ccc2c(c1)c(CCN(C)C)cn2C(=O)CN(C)C ; "HCO2H"] | 2-(Dimethylamino)-1-(3-(2- (dimethylamino)ethyl)-5- methoxy-1H-indol-1-yl)ethan- 1-one formate |
| 553 | 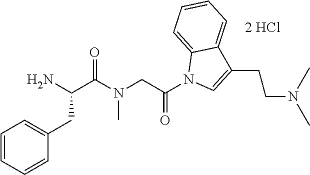 [SMILES: CN(C)CCc1cn(C(=O)CN(C)C(=O)[C@@H](N)Cc2ccccc2)c2ccccc12] | (S)-2-amino-N-(2-(3-(2- (dimethylamino)ethyl)-1H- indol-1-yl)-2-oxoethyl)-N- methyl-3-phenylpropan- amide bis-hydrochloride |
| 554 | 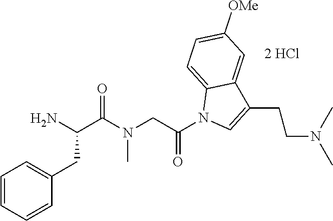 [SMILES: COc1ccc2c(c1)c(CCN(C)C)cn2C(=O)CN(C)C(=O)[C@@H](N)Cc1ccccc1] | (S)-2-amino-N-(2-(3-(2- (dimethylamino)ethyl)-5- methoxy-1H-indol-1-yl)-2- oxoethyl)-N-methyl-3-phen- ylpropanamide bis-hydro- chloride |
| 555 | 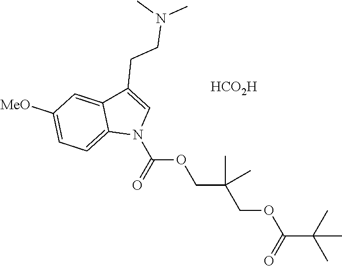 [SMILES: COc1ccc2c(c1)c(CCN(C)C)cn2C(=O)OCC(C)(C)COC(=O)C(C)(C)C ; "HCO2H"] | 2,2-dimethyl-3-(pivaloyloxy)- propyl 3-(2-(dimethylamino)- ethyl)-5-methoxy-1H-indole- 1-carboxylate formate |
| 556 | 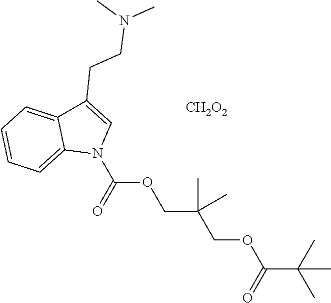 [SMILES: CN(C)CCc1cn(C(=O)OCC(C)(C)COC(=O)C(C)(C)C)c2ccccc12 ; "CH2O2"] | 2,2-Dimethyl-3-(pivaloyloxy)- propyl 3-(2-(dimethylamino)- ethyl)-1H-indole-1-carbox- ylate formate |
| 557 | 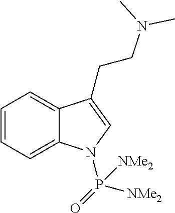 [SMILES: CN(C)CCc1cn(P(=O)(N(C)C)N(C)C)c2ccccc12] | 2-(1-di(dimethylamino)- phosphoryl-indol-3-yl)- N,N-dimethyl-ethanamine |
| 558 | 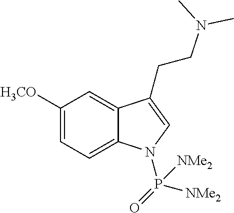 [SMILES: COc1ccc2c(c1)c(CCN(C)C)cn2P(=O)(N(C)C)N(C)C] | 2-(1-di(dimethylamino)- phosphoryl-5-methoxy-indol- 3-yl)-N,N-dimethyl-ethan- amine |
| 170 | 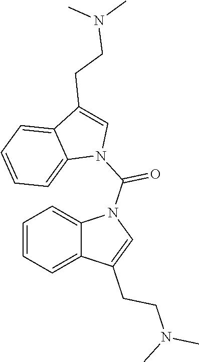 [SMILES: CN(C)CCc1cn(C(=O)n2cc(CCN(C)C)c3ccccc32)c2ccccc12] | bis(3-(2-(Dimethylamino)- ethyl)-1H-indol-1-yl)- methanone di-formate |
| 169 | 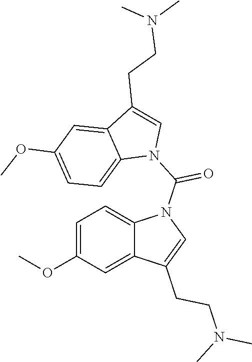 | bis(3-(2-(Dimethylamino)- ethyl)-5-methoxy-1H-indol-1- yl)methanone di-formate |
| 559 | 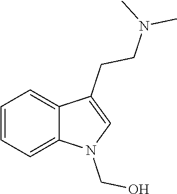 [SMILES: CN(C)CCc1cn(CO)c2ccccc12] | (3-(2-(Dimethylamino)ethyl)- 1H-indol-1-yl)methanol |
| 560 | 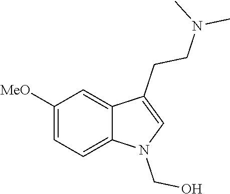 [SMILES: COc1ccc2c(c1)c(CCN(C)C)cn2CO] | (3-(2-(Dimethylamino)ethyl)- 5-methoxy-1H-indol-1-yl)- methanol |
| 187 | 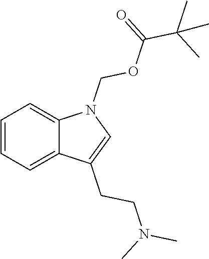 [SMILES: CN(C)CCc1cn(COC(=O)C(C)(C)C)c2ccccc12] | (3-(2-(dimethylamino)ethyl)- 1H-indol-1-yl)methyl pivalate |
| 188 | 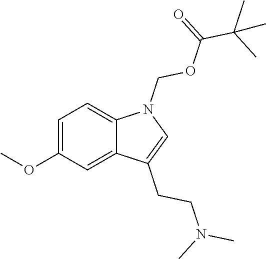 [SMILES: COc1ccc2c(c1)c(CCN(C)C)cn2COC(=O)C(C)(C)C] | (3-(2-(Dimethylamino)ethyl)- 5-methoxy-1H-indol-1-yl)- methyl pivalate |
| 561 | 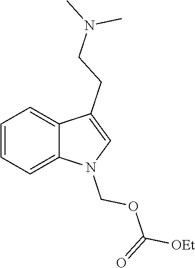 [SMILES: CCOC(=O)OCn1cc(CCN(C)C)c2ccccc21] | (3-(2-(Dimethylamino)ethyl)- 1H-indol-1-yl)methyl ethyl carbonate |
| 562 | 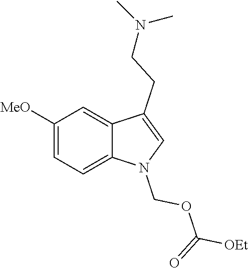 [SMILES: CCOC(=O)OCn1cc(CCN(C)C)c2cc(OC)ccc21] | (3-(2-(Dimethylamino)ethyl)- 5-methoxy-1H-indol-1-yl)- methyl ethyl carbonate |
| 264 | 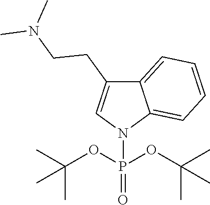 [SMILES: CN(C)CCc1cn(P(=O)(OC(C)(C)C)OC(C)(C)C)c2ccccc12] | Di-tert-butyl ((3-(2-(dimeth- ylamino)ethyl)-1H-indol-1- yl)methyl) phosphate |
| 256 | 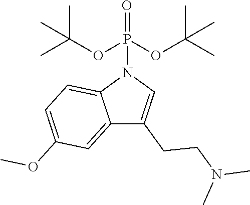 | Di-tert-butyl ((3-(2-(dimeth- ylamino)ethyl)-5-methoxy- 1H-indol-1-yl)methyl)phos- phate |
| 563 | 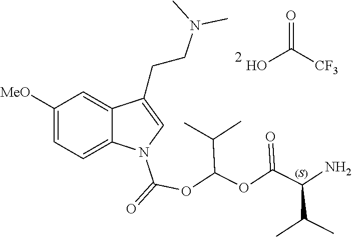 | 1-(((S)-2-amino-3-methyl- butanoyl)oxy)-2-methyl- propyl 3-(2-(dimethylamino)- ethyl)-5-methoxy-1H-indole- 1-carboxylate di-trifluoro- acetate |
| 564 | 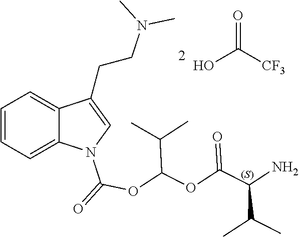 | 1-(((S)-2-amino-3-methyl- butanoyl)oxy)-2-methyl- propyl 3-(2-(dimethylamino)- ethyl)-1H-indole-1-carbox- ylate di-trifluoroacetate |
| 565 | 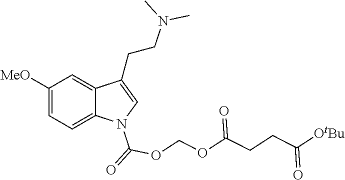 [SMILES: COc1ccc2c(c1)c(CCN(C)C)cn2C(=O)OCOC(=O)CCC(=O)OC(C)(C)C] | tert-Butyl (((3-(2-(dimethyl- amino)ethyl)-5-methoxy-1H- indole-1-carbonyl)oxy)- methyl)succinate |
| 566 | 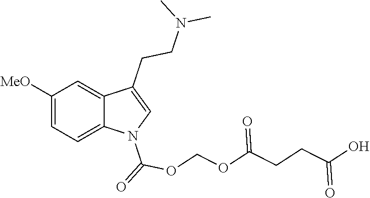 [SMILES: COc1ccc2c(c1)c(CCN(C)C)cn2C(=O)OCOC(=O)CCC(=O)O] | 4-(((3-(2-(Dimethylamino)- ethyl)-5-methoxy-1H-indole- 1-carbonyl)oxy)methoxy)-4- oxobutanoic acid |
| 567 | 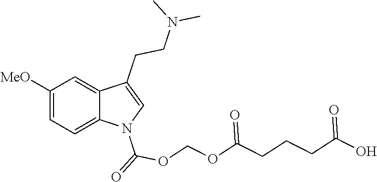 [SMILES: COc1ccc2c(c1)c(CCN(C)C)cn2C(=O)OCOC(=O)CCCC(=O)O] | 5-(((3-(2-(Dimethylamino)- ethyl)-5-methoxy-1H-indole- 1-carbonyl)oxy)methoxy)-5- oxopentanoic acid |
| 568 | 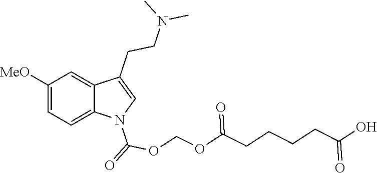 [SMILES: COc1ccc2c(c1)c(CCN(C)C)cn2C(=O)OCOC(=O)CCCCC(=O)O] | 6-(((3-(2-(Dimethylamino)- ethyl)-5-methoxy-1H-indole- 1-carbonyl)oxy)methoxy)-6- oxohexanoic acid |
| 569 | 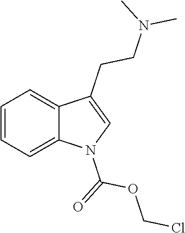 [SMILES: CN(C)CCc1cn(C(=O)OCCl)c2ccccc12] | Chloromethyl 3-(2-(dimethyl- amino)ethyl)-1H-indole-1- carboxylate |
| 570 | 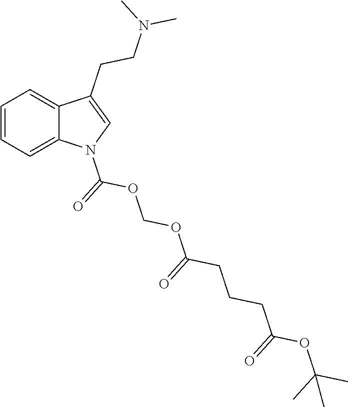 [SMILES: CN(C)CCc1cn(C(=O)OCOC(=O)CCCC(=O)OC(C)(C)C)c2ccccc12] | tert-Butyl (((3-(2-(dimethyl- amino)ethyl)-1H-indole-1- carbonyl)oxy)methyl) glutarate |
| 571 | 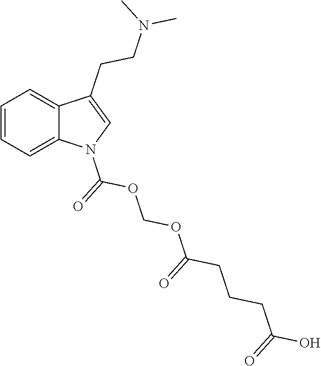 [SMILES: CN(C)CCc1cn(C(=O)OCOC(=O)CCCC(=O)O)c2ccccc12] | 5-(((3-(2-(dimethylamino)- ethyl)-1H-indole-1-carbonyl)- oxy)methoxy)-5-oxopentanoic acid |
| 572 | 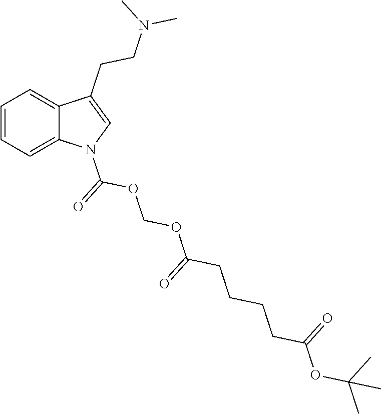 [SMILES: CN(C)CCc1cn(C(=O)OCOC(=O)CCCCC(=O)OC(C)(C)C)c2ccccc12] | tert-Butyl (((3-(2-(dimethyl- amino)ethyl)-1H-indole-1- carbonyl)oxy)methyl) adipate |
| 573 | 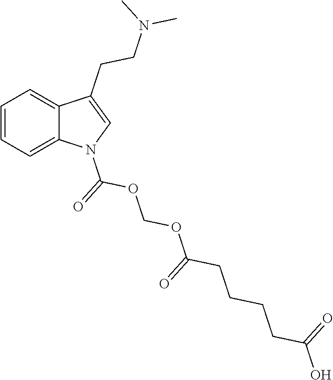 [SMILES: CN(C)CCc1cn(C(=O)OCOC(=O)CCCCC(=O)O)c2ccccc12] | 6-(((3-(2-(Dimethylamino)- ethyl)-1H-indole-1-carbonyl)- oxy)methoxy)-6-oxohexanoic acid |
| 574 | 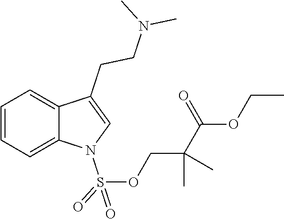 [SMILES: CCOC(=O)C(C)(C)COS(=O)(=O)n1cc(CCN(C)C)c2ccccc21] | Ethyl 3-(((3-(2-(dimethyl- amino)ethyl)-1H-indol-1-yl)- sulfonyl)oxy)-2,2-dimethyl- propanoate |
| 575 | 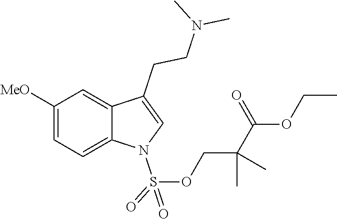 [SMILES: CCOC(=O)C(C)(C)COS(=O)(=O)n1cc(CCN(C)C)c2cc(OC)ccc21] | Ethyl 3-(((3-(2-(dimethyl- amino)ethyl)-5-methoxy-1H- indol-1-yl)sulfonyl)oxy)-2,2- dimethylpropanoate |
| 576 | 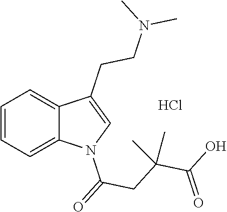 [SMILES: CN(C)CCc1cn(C(=O)CC(C)(C)C(=O)O)c2ccccc12 ; Cl] | 4-(3-(2-(dimethylamino)- ethyl)-1H-indol-1-yl)-2,2- dimethyl-4-oxobutanoic acid HCl salt |
| 577 | 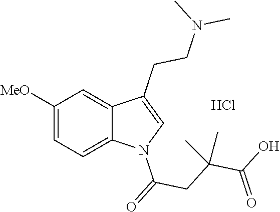 [SMILES: COc1ccc2c(c1)c(CCN(C)C)cn2C(=O)CC(C)(C)C(=O)O ; Cl] | 4-(3-(2-(dimethylamino)- ethyl)-5-methoxy-1H-indol- 1-yl)-2,2-dimethyl-4-oxo- butanoic acid HCl salt |

### Methods of Treatment.

In yet another aspect, the present disclosure provides a method of treating or preventing a disease, disorder, or condition in which an increased level of a tryptamine psychedelic such as DMT is beneficial, comprising administering to a subject in need thereof an effective amount of a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof. In some embodiments, the condition comprises post-traumatic stress disorder, major depression, schizophrenia, Alzheimer's disease, frontotemporal dementia, Parkinson's disease, Parkinson's dementia, dementia, Lewy body dementia, multiple system atrophy, or substance abuse. In some embodiments, the condition comprises musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. The compounds of the present invention can also be used to treat any brain disease.

In some embodiments, a compound disclosed herein has activity as a 5-HT_{2A} modulator. In some embodiments a compound disclosed herein elicits a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity. 5-HT_{2A} antagonists abrogate the neuritogenesis and spinogenesis effects of hallucinogenic compounds with 5-HT_{2A} agonist activity, for example, DMT, LSD, and DOI. In some embodiments, a compound disclosed herein is a 5-HT_{2A} modulator and promotes neural plasticity (e.g., cortical structural plasticity). In some embodiments, a compound disclosed herein is a selective 5-HT_{2A} modulator and promotes neural plasticity (e.g., cortical structural plasticity). Promotion of neural plasticity can include, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. In some embodiments, a compound of the present disclosure functions as a 5-HT_{2A} agonist alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present disclosure to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, flibanserin, nelotanserin and lorcaserin. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior administration of a compound disclosed herein, such as about three or about hours prior administration of the compound. In some embodiments, the serotonin receptor modulator is administered at most about one hour prior to the compound. In some embodiments, the second therapeutic agent is a serotonin receptor modulator. In some embodiments, the serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, a compound of the present disclosure is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments, a compound herein is given to patients in a low dose that is lower than would produce noticeable psychedelic effects but high enough to provide a therapeutic benefit. This dose range is predicted to be between 200 μg (micrograms) and 2 mg.

In some embodiments, a compound described herein is used to treat a neurological disease. For example, a compound provided herein can exhibit, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present disclosure is used for increasing neuronal plasticity. In some embodiments, a compound described herein is used for treating a brain disorder. In some embodiments, a compound described herein is used for increasing translation, transcription, or secretion of neurotrophic factors.

A compound disclosed herein can also be useful for increasing neuronal plasticity in a subject. As used herein, “neuronal plasticity” can refer to the ability of the brain to change structure and/or function throughout a subject's life. New neurons can be produced and integrated into the central nervous system throughout the subject's life. Increasing neuronal plasticity can include, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound the present disclosure can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present disclosure provides a method for increasing neuronal plasticity, comprising contacting a neuronal cell with a compound of the present disclosure. In some embodiments, increasing neuronal plasticity improves a brain disorder described herein.

In some embodiments, a compound disclosed herein is used to increase neuronal plasticity and has, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). Brain disorders can include, for example, migraines, addiction (e.g., substance use disorder), depression, and anxiety.

In some embodiments, the experiment or assay to determine increased neuronal plasticity derived from the administration of any compound of the present disclosure is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present disclosure is a mouse head-twitch response (HTR) assay.

In some embodiments, the condition is a musculoskeletal pain disorder including fibromyalgia, muscle pain, joint stiffness, osteoarthritis, rheumatoid arthritis, muscle cramps. In some embodiments, the present disclosure provides a method of treating a disease of women's reproductive health including premenstrual dysphoric disorder (PMDD), premenstrual syndrome (PMS), post-partum depression, and menopause. In some embodiments, the present disclosure provides a method of treating a brain disorder, including administering to a subject in need thereof, a therapeutically effective amount of a compound of the present disclosure. In some embodiments, the present disclosure provides a method of treating a brain disorder with combination therapy, including administering to a subject in need thereof, a therapeutically effective amount of a compound of the present disclosure and at least one additional therapeutic agent.

In some embodiments, a compound of the present disclosure is used to treat brain disorders. In some embodiments, the compound has, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present disclosure provides a method of treating a brain disorder, comprising administering to a subject in need thereof a therapeutically effective amount of a compound disclosed herein. In some embodiments, the brain disorder is a neurodegenerative disorder, Alzheimer's disease, Parkinson's disease, a psychological disorder, depression, addiction, anxiety, post-traumatic stress disorder, treatment resistant depression, suicidal ideation, major depressive disorder, bipolar disorder, schizophrenia, stroke, traumatic brain injury, or a substance use disorder.

In some embodiments, the brain disorder is a neurodegenerative disorder, Alzheimer's disease or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent. Non-limiting examples of additional therapeutics suitable for administration with a compound of the present disclosure can include lithium, olanzapine (Zyprexa), quetiapine (Seroquel), risperidone (Risperdal), aripiprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Pamate), diazepam (Valium), alprazolam (Xanax), or clonazepam (Klonopin).

In some embodiments, the additional therapeutic agent is a monoamine oxidase inhibitor (MAOI), which can be, for example, moclobemide, caroxazone (Surodil, Timostenil), brofaromine (Consonar), methylene blue, pirlindole (Pirazidol), minaprine (Cantor), metralindole (Inkazan), eprobemide, tetrindole, harmine, harmaline, amiflamine, befloxatone (MD-370,503), cimoxatone (MD-780,515), sercloremine (CGP-4718-A), esuprone, or CX157.

In some embodiments, the additional therapeutic agent is a phenethylamine, such as 3,4-methylene-dioxymethamphetamine (MDMA) and analogs thereof. Other suitable empathogenic agents for use in combination a compound of the present disclosure include, without limitation, N-Allyl-3,4-methylenedioxy-amphetamine (MDAL), N-Butyl-3,4-methylenedioxyamphetamine (MDBU), N-Benzyl-3,4-methylenedioxyamphetamine (MDBZ), N-Cyclopropylmethyl-3,4-methylenedioxy amphetamine (MDCPM), N,N-Dimethyl-3,4-methylenedioxyamphetamine (MDDM), N-Ethyl-3,4-methylenedioxyamphetamine (MDE; MDEA); N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC) N-Methoxy-3,4-methylenedioxyamphetamine (MDMEO), N-(2-Methoxyethyl)-3,4-methylenedioxyamphetamine (MDMEOET), alpha,alpha,N-Trimethyl-3,4-methylenedioxyphenethylamine (MDMP), 3,4-Methylenedioxy-N-methylphentermine N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine), N-Propargyl-3,4-methylenedioxyamphetamine (MDPL), Methylenedioxy-2-aminoindane (MDAI), 1,3-Benzodioxolyl-N-methylbutanamine (MBDB), N-methyl-1,3-benzodioxolylbutanamine, 3,4-methylenedioxy-N-methyl-α-ethylphenylethylamine, 3,4-Methylenedioxyamphetamine (MDA), Methylone (3,4-methylenedioxy-N-methylcathinone), Ethylone (3,4-methylenedioxy-N-ethylcathinone), GHB or Gamma Hydroxybutyrate or sodium oxybate, N-Propyl-3,4-methylenedioxyamphetamine (MDPR), and the like.

In some embodiments, a compound of the present disclosure is used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors.

As used herein, the term “neurotrophic factor” can refer to a family of soluble peptides or proteins which support the survival, growth, and differentiation of developing and mature neurons. Increasing at least one of translation, transcription, or secretion of neurotrophic factors can be useful for, for example, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors increases neuronal plasticity. In some embodiments, increasing at least one of translation, transcription, or secretion of neurotrophic factors promotes neuronal growth, promotes neuritogenesis, promotes synaptogenesis, promotes dendritogenesis, increases dendritic arbor complexity, and/or increases dendritic spine density.

In some embodiments, a 5-HT_{2A} modulators (e.g., 5-HT_{2A} agonists) is used to increase at least one of translation, transcription, or secretion of neurotrophic factors. In some embodiments, a compound of the present disclosure is used to increase translation, transcription, or secretion of neurotrophic factors. In some embodiments, increasing translation, transcription or secretion of neurotrophic factors is sufficient for the treatment of migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, or addiction (e.g., substance use disorder).

An experiment or assay can be used to detect increased translation of neurotrophic factors, which can include, for example, ELISA, western blot, an immunofluorescence assay, a proteomic experiment, and mass spectrometry. In some embodiments, the experiment or assay used to detect increased transcription of neurotrophic factors is a gene expression assay, PCR, or microarray. In some embodiments, the experiment or assay used to detect increased secretion of neurotrophic factors is ELISA, western blot, an immunofluorescence assay, a proteomic experiment, or a mass spectrometry assay.

In some embodiments, the present disclosure provides a method for increasing translation, transcription, or secretion of neurotrophic factors, wherein the method comprises contacting a neuronal cell with a compound disclosed herein.

### Pharmacokinetics.

In yet another aspect, the present disclosure provides a method of treating a condition in a subject in need thereof, the method comprising administering to the subject a therapeutically-effective amount of a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik3), (Il), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof. In some embodiments, a plasma concentration of DMT in the subject is, for administration of a dose of about 10 mg/kg, from about 500 nM to about 2500 ng/mL at about 0.25 hours after the administration. In some embodiments, a plasma concentration of DMT in the subject is, for administration of a dose of about 10 mg/kg, from about 1400 nM to about 2500 ng/mL at about 0.5 hours after the administration. In some embodiments, a plasma concentration of DMT in the subject is, for administration of a dose of about 10 mg/kg, from about 1400 nM to about 2500 ng/mL at about 0.75 hours after the administration. In some embodiments, a plasma concentration of DMT in the subject is, for administration of a dose of about 10 mg/kg, from about 1100 nM to about 2500 ng/mL at about 1 hours after the administration. In some embodiments, a plasma concentration of DMT in the subject is, for administration of a dose of about 10 mg/kg, from about 600 nM to about 2500 ng/mL at about 2 hours after the administration. In some embodiments, a plasma concentration of DMT in the subject is, for administration of a dose of about 10 mg/kg, from about 50 nM to about 2500 ng/mL at about 4 hours after the administration. In some embodiments, a plasma concentration of DMT in the subject is, for administration of a dose of about 10 mg/kg, from about 500 nM to about 2500 ng/mL at about 0.25 hours after the administration. In some embodiments, a plasma concentration of DMT in the subject is, for administration of a dose of about 10 mg/kg, from about 1400 nM to about 1800 ng/mL at about 0.5 hours after the administration. In some embodiments, a plasma concentration of DMT in the subject is, for administration of a dose of about 10 mg/kg, from about 1400 nM to about 2400 ng/mL at about 0.75 hours after the administration. In some embodiments, a plasma concentration of DMT in the subject is, for administration of a dose of about 10 mg/kg, from about 1100 nM to about 1600 ng/mL at about 1 hours after the administration. In some embodiments, a plasma concentration of DMT in the subject is, for administration of a dose of about 10 mg/kg, from about 600 nM to about 1200 ng/mL at about 2 hours after the administration. In some embodiments, a plasma concentration of DMT in the subject is, for administration of a dose of about 10 mg/kg, from about 50 nM to about 1000 ng/mL at about 4 hours after the administration. In some embodiments, the administration is oral administration. In some embodiments, the subject is a rat. In some embodiments, the compound is a compound of Formula (I). In some embodiments, the subject is a rat. In some embodiments, the compound is a compound of Formula (I), wherein R³ is cycloalkyl or alkyl. In some embodiments, the compound is a compound of Formula (I), wherein R¹ is hydrogen.

In yet another aspect, the present disclosure provides a method of treating a condition in a subject in need thereof, the method comprising administering to the subject a therapeutically-effective amount of a compound of Formula (I), (Ia), (Ib), (Ib-1) (Ib1), (Ic), (Id), (Ie), (If), (If1), (Ig), (Ih), (Ii), (Ij), (Ik), (Ik1), (Ik2), (Ik), (I), (Im), (Im1), (Im1a), (In), (In1), (Io), (Io1), (Io2), (Io1a), (Ip) (Ip1), (Iq), (Iq1), (Ir), (Ir1), (Is), (It), (Iu), (Iv), or (Iw), or a pharmaceutically acceptable salt thereof. In some embodiments, a plasma concentration of 5-OMe-DMT in the subject is, for administration of a dose of about 10 mg/kg, from about 50 nM to about 300 ng/mL at about 0.25 hours after the administration. In some embodiments, a plasma concentration of 5-OMe-DMT in the subject is, for administration of a dose of about 10 mg/kg, from about 100 nM to about 300 ng/mL at about 0.5 hours after the administration. In some embodiments, a plasma concentration of 5-OMe-DMT in the subject is, for administration of a dose of about 10 mg/kg, from about 100 nM to about 300 ng/mL at about 0.75 hours after the administration. In some embodiments, a plasma concentration of 5-OMe-DMT in the subject is, for administration of a dose of about 10 mg/kg, from about 100 nM to about 300 ng/mL at about 1 hours after the administration. In some embodiments, a plasma concentration of 5-OMe-DMT in the subject is, for administration of a dose of about 10 mg/kg, from about 30 nM to about 300 ng/mL at about 2 hours after the administration. In some embodiments, a plasma concentration of 5-OMe-DMT in the subject is, for administration of a dose of about 10 mg/kg, from about 1 nM to about 300 ng/mL at about 4 hours after the administration. In some embodiments, a plasma concentration of 5-OMe-DMT in the subject is, for administration of a dose of about 10 mg/kg, from about 50 nM to about 150 ng/mL at about 0.25 hours after the administration. In some embodiments, a plasma concentration of 5-OMe-DMT in the subject is, for administration of a dose of about 10 mg/kg, from about 100 nM to about 300 ng/mL at about 0.5 hours after the administration. In some embodiments, a plasma concentration of 5-OMe-DMT in the subject is, for administration of a dose of about 10 mg/kg, from about 100 nM to about 200 ng/mL at about 0.75 hours after the administration. In some embodiments, a plasma concentration of 5-OMe-DMT in the subject is, for administration of a dose of about 10 mg/kg, from about 100 nM to about 250 ng/mL at about 1 hours after the administration. In some embodiments, a plasma concentration of 5-OMe-DMT in the subject is, for administration of a dose of about 10 mg/kg, from about 30 nM to about 100 ng/mL at about 2 hours after the administration. In some embodiments, a plasma concentration of 5-OMe-DMT in the subject is, for administration of a dose of about 10 mg/kg, from about 1 nM to about 200 ng/mL at about 4 hours after the administration. In some embodiments, the administration is oral administration. In some embodiments, the subject is a rat. In some embodiments, the compound is a compound of Formula (I). In some embodiments, the subject is a rat. In some embodiments, the compound is a compound of Formula (I), wherein R³ is cycloalkyl or alkyl. In some embodiments, the compound is a compound of Formula (I), wherein R¹ is methoxy.

### EXAMPLES

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMR. Abbreviations used are those conventional in the art. If not defined, the terms have their generally accepted meanings.

### Example 1: Preparation of Selected Compounds and Intermediates

The following preparations of compounds and intermediates are given to enable those of skill in the art to more clearly understand and to practice the present disclosure. They should not be considered as limiting the scope of the disclosure, but merely as illustrative and representative thereof.

### Abbreviation

app apparent
Boc tert-butyl carbamate
Boc-Sar-OH Boc-sarcosine
br broad
CDCl₃ d₃-chloroform
d doublet
dd doublet of doublets
DCM dichloromethane
DIPEA diisopropylethylamine
DMA dimethylacetamide
DMAP 4-dimethylaminopyridine
DMF N,N-dimethylformamide
DMSO dimethyl sulfoxide
EtOAc ethyl acetate
HATU 1-[Bis(dimethylamino)methylene]-1H-1,2,3-triazolo[4,5-b]pyridinium 3-oxid hexafluorophosphate
HCl hydrochloric acid
h hextet; sextet
hr or hrs hour or hours
HPLC high pressure liquid chromatography
LC-MS liquid chromatography and mass spectrometry
MeOH MeOH
MeCN acetonitrile
MS mass spectrometry
m multiplet
mm(s) minute(s)
mL milliliter(s)
μL microliter(s)
m/z mass to charge ratio
p pentet
q quartet
NaHCO₃ sodium hydrogen carbonate
Na₂SO₄ sodium sulfate
NMP N-methyl-2-pyrrolidone
NMR nuclear magnetic resonance
Rt retention time
s singlet
sar sarcosine
t triplet
tert tertiary
THF tetrahydrofuran

Referring to the examples that follow, compounds of the preferred embodiments were synthesized using the methods described herein, or other methods, which are known in the art. The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification.

### General Conditions for Characterization:

NMR analysis.

¹H, ¹³C, ¹⁹F and ³¹P NMR analyses were conducted on a Bruker™ Avance 400 MHz NMR spectrometer using deuterated chloroform or deuterated dimethyl sulfoxide as solvent. The shift (d) of each signal was measured in parts per million (ppm) relative the residual solvent peak, and the multiplicity reported together with the associated coupling constant (J), where applicable.

### UPLC-MS Analysis Methodology.

UPLC-MS analysis was carried out on a Waters™ Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector. A Waters™ Acquity UPLC BEH C18 column (2.1×50 mm, 1.7 mm) was used to separate the analytes.

Samples were prepared by dissolution (with or without sonication) into 1 mL of 50% (v/v) MeCN in water. The resulting solutions were then filtered through a 0.2 mm syringe filter before submitting for analysis. All of the solvents, including formic acid and 36% ammonia solution, were purchased as the HPLC grade.

Conditions (Acidic 2 min).

0.1% v/v Formic acid in water [Eluent A]; 0.1% v/v Formic acid in MeCN [Eluent B]; flow rate 0.8 mL/min; column oven 50° C.; sample manager 20° C.; injection volume 2 mL and 1.5 minutes equilibration time between samples. Gradient parameters are provided in TABLE 2.

TABLE 2

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Acidic 4 min).

0.1% v/v formic acid in water [Eluent A]; 0.1% v/v formic acid in MeCN [Eluent B]; flow rate 0.8 mL/min; column oven 50° C.; sample manager 20° C.; injection volume 2 mL and 1.5 minutes equilibration time between samples. Gradient parameters are provided in TABLE 3.

TABLE 3

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Acidic 6 min).

0.100 v/v formic acid in water [Eluent A]; 0.1% v/v formic acid in MeCN [Eluent B]; flow rate 0.8 mL/min; column oven 50° C.; sample manager 20° C.; injection volume 2 mL and 1.5 minutes equilibration time between samples. Gradient parameters are provided in TABLE 4.

TABLE 4

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

Conditions (Basic 2 min).

0.1% ammonia in water [Eluent A]; 0.1% ammonia in MeCN [Eluent B]; flow rate 0.8 mL/min; column oven 50° C.; sample manager 20° C.; injection volume 2 mL and 1.5 minutes equilibration time between samples. Gradient parameters are provided in TABLE 5.

TABLE 5

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

Conditions (Basic 4 min).

0.1% ammonia in water [Eluent A]; 0.1% ammonia in MeCN [Eluent B]; flow rate 0.8 mL/min; column oven 50° C.; sample manager 20° C.; injection volume 2 mL and 1.5 minutes equilibration time between samples. Gradient parameters are provided in TABLE 6.

TABLE 6

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

Conditions (Basic 6 min).

0.1% ammonia in water [Eluent A]; 0.1% ammonia in MeCN [Eluent B]; flow rate 0.8 mL/min; column oven 50° C.; sample manager 20° C.; injection volume 2 mL and 1.5 minutes equilibration time between samples. Gradient parameters are provided in TABLE 7.

TABLE 7

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

### Example 1-1: Dimethyl Tryptamine (DMT)

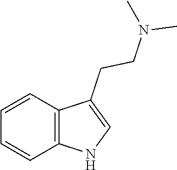

A solution of 4% sulfuric acid (0.16 M, 0.82 mL, 15.3 mmol) was heated to 55° C. and purged with nitrogen. Phenylhydrazine (1.50 g, 13.9 mmol) was added to the heated acidic solution, followed by dropwise addition of 4,4-dimethoxy-N,N-dimethyl-butan-1-amine (2.46 g, 15.3 mmol), while maintaining 55° C. The resulting solution was heated to reflux for 2 h and then cooled to room temperature. A solution of NaOH (10 g) in H₂O (50 mL) was added slowly to the crude reaction mixture, which was then extracted with EtOAc (×3). The organic phases were combined, washed with brine, dried over Na₂SO₄, filtered, and concentrated to dryness under reduced pressure to produce an orange oil (2.1 g). The crude oil was purified by column chromatography on silica gel (40 g cartridge, 5-20% MeOH in acetone) to afford 2-(1H-indol-3-yl)-N,N-dimethyl-ethanamine (1.46 g, 53% yield) as a solid. ¹H NMR (400 MHz, d₆-DMSO) δ 10.77 (s, 1H), 7.51 (ddt, J=7.9, 1.5, 0.9 Hz, 1H), 7.33 (dt, J=8.1, 1.0 Hz, 1H), 7.16-7.12 (m, 1H), 7.06 (ddd, J=8.2, 7.0, 1.2 Hz, 1H), 6.97 (ddd, J=7.9, 7.0, 1.1 Hz, 1H), 2.86-2.77 (m, 2H), 2.55-2.50 (m, 2H), 2.23 (s, 6H).

### Example 1-2: 5-Methoxy Dimethyl Tryptamine (5-MeO-DMT)

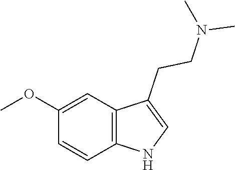

To a magnetically stirred solution of 4-methoxyphenylhydrazine hydrochloride (2.00 g, 11.5 mmol) in water (20 mL) at room temperature under an atmosphere of N2 was added H₂SO₄ (0.67 mL, 12.6 mmol) dropwise while maintaining the temperature below 40° C. The solution was heated to 40° C. and stirred for 10 min. A mixture of 4,4-dimethoxy-N,N-dimethyl-butan-1-amine (2.20 mL, 12.0 mmol) in acetonitrile (10 mL) was added dropwise. The reaction was agitated at 40° C. for 1 h. The acetonitrile was removed under reduced pressure, and the resulting aqueous solution was washed with 2-MeTHF (2×30 mL). The aqueous phase was treated with NaOH (4 M, 9.00 mL, 1.60 g NaOH) to adjust the pH to ˜ 11-12, and the product was extracted with 2-MeTHF (3×30 mL). The organic phases were combined and concentrated under reduced pressure to provide a brown oil, which was then purified by column chromatography on silica gel (20 g cartridge, 1-10% MeOH in acetone) to afford 2-(5-methoxy-1H-indol-3-yl)-N,N-dimethyl-ethanamine (1.70 g, 68% yield) as an oil. UPLC-MS (4 min, basic): rt=1.12 min; m/z=219.2 [M+H]⁺; rt=1.12 min; m/z=219.2 [M+H]⁺; —two peaks same product; ¹H NMR (400 MHz, CDCl₃) δ 7.97 (s, 1H), 7.29 (dd, J=4.8, 1.4 Hz, 1H), 7.09 (d, J=2.4 Hz, 1H), 7.04 (d, J=2.3 Hz, 1H), 6.89 (dt, J=8.8, 1.9 Hz, 1H), 3.90 (s, 3H), 2.99-2.90 (m, 2H), 2.72-2.61 (m, 2H), 2.38 (s, 6H).

### Example 1-3: Ethyl 3-[2-(dimethylamino)ethyl]indole-1-carboxylate (Compound 20)

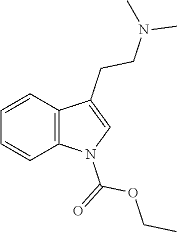 [SMILES: CCOC(=O)n1cc(CCN(C)C)c2ccccc21]

To a stirring solution of DMT (2-(1H-indol-3-yl)-N,N-dimethyl-ethanamine, 99 mg, 0.53 mmol) in THF (10 mL) at −78° C. was added sodium bis(trimethylsilyl)amide (2.0 M solution in THF, 0.53 mL, 1.05 mmol). The resulting solution was stirred at −78° C. for 15 min. Ethyl chloroformate (101 μL, 1.05 mmol) was added dropwise and stirred for a further 5 min at −78° C. The reaction mixture was allowed to warm to room temperature and then stirred for 18 h. Saturated brine was added followed by EtOAc. The organic phase was separated and the aqueous was extracted with EtOAc (×2). The organic phases were combined, washed with brine, dried over Na₂SO₄, filtered, and evaporated to provide an orange oil. The crude oil was purified by column chromatography on silica gel (4 g, 0 to 20% methanol in dichloromethane) to afford Compound 20 (ethyl 3-[2-(dimethylamino)ethyl]indole-1-carboxylate, 77 mg, 56% yield) as an oil. UPLC-MS (4 min, basic): rt=1.84 min; m/z=261.0 [M+H]⁺; ¹H NMR (400 MHz, CDCl₃) δ 8.00 (d, J=8.2 Hz, 1H), 7.39 (ddd, J=7.8, 1.4, 0.8 Hz, 1H), 7.28 (s, 1H), 7.18 (ddd, J=8.3, 7.2, 1.4 Hz, 1H), 7.15-7.06 (m, 1H), 4.32 (q, J=7.1 Hz, 2H), 2.79-2.69 (m, 2H), 2.59-2.46 (m, 2H), 2.21 (s, 6H), 1.31 (t, J=7.1 Hz, 3H); ¹³C NMR (101 MHz, CDCl₃) δ 151.0, 135.6, 130.6, 124.6, 122.7, 122.3, 119.4, 119.0, 115.3, 77.4, 77.0, 76.7, 63.0, 59.2, 45.4, 23.3, 14.5.

### Example 1-4: Ethyl 3-[2-(dimethylamino)ethyl]-5-methoxy-indole-1-carboxylate (Compound 19)

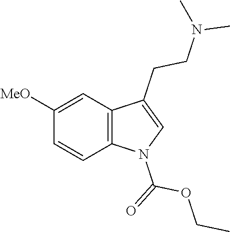 [SMILES: CCOC(=O)n1cc(CCN(C)C)c2cc(OC)ccc21]

To a stirring solution of 5-MeO DMT (2-(5-methoxy-1H-indol-3-yl)-N,N-dimethyl-ethanamine, 200 mg, 0.92 mmol) in THF (10 mL) at −78° C. was added sodium bis(trimethylsilyl)amide (2.0 M solution in THF, 0.69 mL, 1.37 mmol). The resulting solution was stirred at −78° C. for 15 min. Ethyl chloroformate (180 μL, 1.83 mmol) was added dropwise and stirred for a further 5 min at −78° C. The reaction mixture was allowed to warm to room temperature and then stirred for 18 h. The reaction mixture was then diluted with EtOAc (10 mL), washed with H₂O (10 ml), and extracted a second time with EtOAc (10 mL). The combined organic phases were washed with brine (10 mL), dried over Na₂SO₄, and concentrated under reduced pressure. The crude product was purified by column chromatography on silica gel (10 g, 50-100% EtOAc in heptane with 1% TEA over 10 CV, then 100% ethyl acetate with 1% TEA for 10 CV) to give Compound 19 (ethyl 3-[2-(dimethylamino)ethyl]-5-methoxy-indole-1-carboxylate, 115 mg, 43% yield) as an oil. UPLC-MS (4 min, basic): rt=1.79 min; m/z=291.2 [M+H]⁺; 100%. ¹H NMR (400 MHz, CDCl₃) δ 8.03 (s, 1H), 7.41 (s, 1H), 6.99 (d, J=2.4 Hz, 1H), 6.93 (dd, J=9.0, 2.5 Hz, 1H), 4.45 (q, J=7.1 Hz, 2H), 3.87 (s, 3H), 2.88-2.78 (m, 2H), 2.67-2.58 (m, 2H), 2.33 (s, 6H), 1.45 (t, J=7.1 Hz, 3H).

### Example 1-5: 2-(1-Diisopropoxyphosphorylindol-3-yl)-N,N-dimethyl-ethanamine (Compound 263)

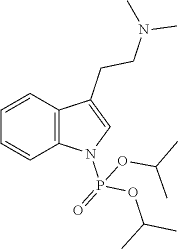 [SMILES: CC(C)OP(=O)(OC(C)C)n1cc(CCN(C)C)c2ccccc21]

To a stirring solution of DMT (2-(1H-indol-3-yl)-N,N-dimethyl-ethanamine, 200 mg, 1.06 mmol) in THF (10 mL) at −78° C. was added sodium bis(trimethylsilyl)amide (1.0 M solution in THF, 1.6 mL, 1.59 mmol). The mixture was stirred at −78° C. for 15 min, and 2-[chloro(isopropoxy)phosphoryl]oxypropane (0.100 mL, 0.6 mmol) was then added. The mixture allowed to warm to room temperature, stirred for 20 h, and concentrated under reduced pressure. The resulting residue was first purified by column chromatography on silica gel (12 g Si, 5 CV DCM+1% TEA, 10 CV 0-5% iso-propanol in DCM+1% TEA, 20 CV 10% iso-propanol in DCM+1% TEA), then further purified by preparative-HPLC to give Compound 263 (2-(1-diisopropoxyphosphorylindol-3-yl)-N,N-dimethyl-ethanamine, 123 mg, 33% yield) as an oil. UPLC-MS (4 min, basic): rt=1.88 min, m/z=353.3 [M+H]⁺; ¹H NMR (400 MHz, CDCl₃) δ 7.71 (dt, J=8.2, 0.9 Hz, 1H), 7.57 (dtd, J=7.5, 1.6, 0.7 Hz, 1H), 7.28 (dd, J=7.1, 1.3 Hz, 1H), 7.25-7.19 (m, 2H), 4.63 (dp, J=7.5, 6.2 Hz, 2H), 2.92-2.86 (m, 2H), 2.68-2.60 (m, 2H), 2.33 (s, 6H), 1.41 (d, J=6.2 Hz, 6H), 1.10 (d, J=6.2 Hz, 6H); ³¹P NMR (162 MHz, CDCl₃) δ −5.27 (t, J=7.7 Hz).

### Example 1-6: 2-(1-Diisopropoxyphosphoryl-5-methoxy-indol-3-yl)-N,N-dimethyl-ethanamine (Compound 255)

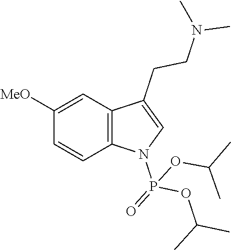 [SMILES: COc1ccc2c(c1)c(CCN(C)C)cn2P(=O)(OC(C)C)OC(C)C]

To a stirring solution of 5-MeO DMT (2-(5-methoxy-1H-indol-3-yl)-N,N-dimethyl-ethanamine, 228 mg, 1.04 mmol) in THF (10 mL) at −78° C. was added sodium bis(trimethylsilyl)amide (1.0 M solution in THF, 1.6 mL, 1.57 mmol), whereupon the mixture was stirred at −78° C. for 15 min. 2-[chloro(isopropoxy)phosphoryl]oxypropane (0.37 mL, 2.1 mmol) was then added, and the mixture allowed to warm to room temperature. The mixture was stirred at temperature for 20 h, quenched with iso-propanol (5 mL), and concentrated under reduced pressure. The residue was purified by column chromatography on silica gel (12 g Si, 5 CV DCM+1% TEA, 10 CV 0-5% iso-propanol in DCM+1% TEA, 20 CV 10% iso-propanol in DCM+1% TEA), and then further purified by preparative-HPLC to afford Compound 255 (2-(1-diisopropoxyphosphoryl-5-methoxy-indol-3-yl)-N,N-dimethyl-ethanamine, 65 mg, 16% yield) as an oil. UPLC-MS (4 min, basic): rt=1.82 min, m/z=383.3 [M+H]⁺; ¹H NMR (400 MHz, CDCl₃) δ 7.59 (d, J=8.9 Hz, 1H), 7.24 (dt, J=2.2, 1.1 Hz, 1H), 7.00 (t, J=2.0 Hz, 1H), 6.90 (dd, J=8.9, 2.5 Hz, 1H), 4.61 (dhept, J=7.5, 6.2 Hz, 2H), 3.86 (s, 3H), 2.89-2.81 (m, 2H), 2.67-2.58 (m, 2H), 2.34 (s, 6H), 1.40 (d, J=6.2 Hz, 6H), 1.10 (d, J=6.2 Hz, 6H).

### Example 1-7: Tert-butyl [3-[2-(dimethylamino)ethyl]indol-1-yl]methyl hydrogen (Compound 511)

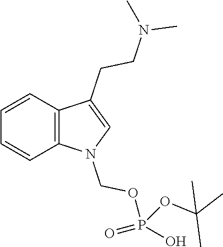 [SMILES: CN(C)CCc1cn(COP(=O)(O)OC(C)(C)C)c2ccccc12]

To a stirring solution of DMT (2-(1H-indol-3-yl)-N,N-dimethyl-ethanamine, 150 mg, 0.8 mmol) in DMSO (3 mL) at room temperature was added K₂CO₃, 325 mesh (440 mg, 3.2 mmol). The mixture was stirred at room temperature for 15 min, after which time di-tert-butyl chloromethyl phosphate (412 mg, 1.59 mmol) was added and the mixture stirred for 17 h. H₂O (2 mL) was then added, and the mixture was stirred for 21 h at rt. The resulting crude mixture was purified by reverse phase column chromatography (23 g, gradient of 10-50% MeCN in water with 0.1% NH₄OH) to afford Compound 511 (tert-butyl [3-[2-(dimethylamino)ethyl]indol-1-yl]methyl hydrogen phosphate, 219 mg, 78% yield) as a solid. UPLC-MS (2 min, basic): rt=0.74 min, m/z=355.1 [M+H]⁺; ¹H NMR (400 MHz, d₆-DMSO) δ 7.68 (dt, J=7.9, 1.1 Hz, 1H), 7.54 (dt, J=8.3, 0.9 Hz, 1H), 7.33 (s, 1H), 7.18 (ddd, J=8.2, 7.0, 1.2 Hz, 1H), 7.13-6.98 (m, 1H), 6.49 (s, 1H), 4.86 (dd, J=8.9, 3.4 Hz, 2H), 3.55-3.41 (m, 2H), 3.33 (s, 6H), 3.18 (td, J=8.0, 3.1 Hz, 2H), 3.08 (d, J=1.8 Hz, 6H), 1.31 (d, J=0.8 Hz, 9H); ³¹P NMR (162 MHz, d₆-DMSO) δ −6.09 (q, J=8.8 Hz).

### Example 1-8: Tert-butyl [3-[2-(dimethylamino)ethyl]-5-methoxy-indol-1-yl]methyl hydrogen phosphate (Compound 510)

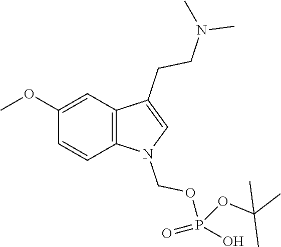 [SMILES: COc1ccc2c(c1)c(CCN(C)C)cn2COP(=O)(O)OC(C)(C)C]

To a stirring solution of 5-MeO DMT (2-(5-methoxy-1H-indol-3-yl)-N,N-dimethyl-ethanamine, 150 mg, 0.69 mmol) in DMSO (3 mL) at room temperature was added K₂CO₃, 325 mesh (380 mg, 2.75 mmol). The mixture was stirred at room temperature for 15 min, and then chloromethyl bis(2-methyl-2-propanyl) phosphate (356 mg, 1.37 mmol) was added. The resulting mixture was stirred at room temperature for 17 h, after which time H₂O (2 mL) was added and the mixture was stirred for a further 21 h at room temperature. The mixture was purified by reverse phase column chromatography (C18, 23 g, 10-50% MeCN in H₂O with 0.1% NH₄OH) to afford Compound 510 (tert-butyl [3-[2-(dimethylamino)ethyl]-5-methoxy-indol-1-yl]methyl hydrogen phosphate, 195 mg, 74% yield) as a solid. UPLC-MS (2 min, basic): rt=0.75 min, m/z=385.2 [M+H]⁺; ¹H NMR (400 MHz, d₆-DMSO) δ 7.44-7.14 (m, 3H), 6.83-6.70 (m, 1H), 5.41 (s, 1H), 4.85 (d, J=9.0 Hz, 2H), 3.80 (d, J=5.4 Hz, 3H), 3.50-3.41 (m, 2H), 3.14 (d, J=8.5 Hz, 2H), 3.07 (s, 6H), 1.30 (s, 9H); ³¹P NMR (162 MHz, d₆-DMSO) δ −5.99 (q, J=8.8 Hz).

### Example 1-9: Isobutyl 3-[2-(dimethylamino)ethyl]-6-methoxy-indole-1-carboxylate (Compound 517)

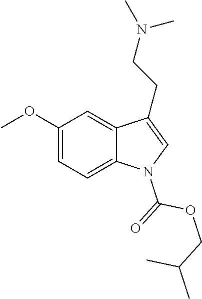 [SMILES: COc1ccc2c(c1)c(CCN(C)C)cn2C(=O)OCC(C)C]

To a stirring solution of 5-OMe-DMT (200 mg, 0.92 mmol) in THF (10 mL) at −78° C. was added NaHMDS, 1M in THF (1.4 mL, 1.4 mmol). The mixture was stirred at −78° C. for 15 min before isobutyl chloroformate (0.24 mL, 1.83 mmol) was added. The mixture was allowed to warm to rt and stirred for 30 min. The mixture was diluted with EtOAc (10 mL), washed with H₂O (10 mL), brine (10 mL), dried over Na₂SO₄, filtered and the filtrate was concentrated under reduced pressure. The crude residue was purified by column chromatography on silica gel (12 g cartridge) eluting with a gradient of EtOAc (50% to 100%; v/v) in hexane (with 100 NEt₃) to afford isobutyl 3-[2-(dimethylamino)ethyl]-6-methoxy-indole-1-carboxylate (Compound 517, 56 mg, 19% yield) as an oil. UPLC-MS (4 min, basic): rt=2.17 min, m/z=319.1 [M+H]⁺; ¹H NMR (400 MHz, CDCl₃) δ 7.96 (s, 1H), 7.34 (s, 1H), 6.92 (d, J=2.5 Hz, 1H), 6.87 (dd, J=9.0, 2.5 Hz, 1H), 4.12 (d, J=6.6 Hz, 2H), 3.80 (s, 3H), 2.81-12.72 (m, 2H), 2.60-2.51 (m, 2H), 2.27 (s, 6H), 2.15-2.00 (m, 1H), 0.98 (d, J=6.7 Hz, 6H).

The following compounds were made by analogous methods to that described for isobutyl 3-[2-(dimethylamino)ethyl]-6-methoxy-indole-1-carboxylate (Compound 517)

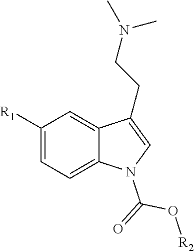

| Cpd | Name | R₁ | R₂ | UPLC-MS | ¹H NMR |
|---|---|---|---|---|---|
| 518 | tert-butyl 3-[2- | H | ^{t}Bu | (4 min, | ¹H NMR (400 MHz, |
| | (dimethylamino)- | | | basic): rt = | CDCl₃) δ 8.15-8.08 (m, |
| | ethyl]indole-1- | | | 2.13 min, | 1H), 7.53 (ddd, J = 7.7, 1.4, |
| | carboxylate | | | m/z = | 0.8 Hz, 1H), 7.40 (s, 1H), |
| | | | | 289.1.1 | 7.31 (ddd, J = 8.3, 7.2, 1.4 |
| | | | | [M + H]⁺, | Hz, 1H), 7.23 (ddd, J = 8.2, |
| | | | | 96% purity. | 7.3, 1.1 Hz, 1H), 2.91-2.82 |
| | | | | | (m, 2H), 2.68-2.59 (m, |
| | | | | | 2H), 2.33 (s, 6H), 1.67 (s, |
| | | | | | 9H). |
| 519 | isopropyl 3-[2- | H | ⁱPr | (4 min, | ¹H NMR (400 MHz, |
| | (dimethylamino)- | | | basic): rt = | CDCl₃) δ 8.15 (d, J = 8.2 |
| | ethyl]indole-1- | | | 1.90 min, | Hz, 1H), 7.54 (ddd, J = 7.7, |
| | carboxylate | | | m/z = 275.1 | 1.4, 0.8 Hz, 1H), 7.47-7.36 |
| | | | | [M + H]⁺, | (m, 1H), 7.32 (ddd, J = 8.4, |
| | | | | 97% purity. | 7.2, 1.3 Hz, 1H), 7.25 (td, J = |
| | | | | | 7.5, 1.1 Hz, 1H), 5.26 |
| | | | | | (hept, J = 6.3 Hz, 1H), 2.92- |
| | | | | | 2.83 (m, 2H), 2.68-2.60 |
| | | | | | (m, 2H), 2.34 (s, 6H), 1.45 |
| | | | | | (d, J = 6.3 Hz, 6H). |
| 520 | propyl 3-[2- | H | Pr | (4 min, | ¹H NMR (400 MHz, |
| | (dimethylamino)- | | | basic): rt = | CDCl₃) 8 8.15 (d, J = 8.0 |
| | ethyl]indole-1- | | | 2.02 min, | Hz, 1H), 7.58-7.49 (m, |
| | carboxylate | | | m/z = 275.1 | 1H), 7.43 (s, 1H), 7.33 |
| | | | | [M + H]⁺, | (ddd, J = 8.4, 7.2, 1.4 Hz, |
| | | | | 96% purity. | 1H), 7.25 (td, J = 7.5, 1.1 |
| | | | | | Hz, 1H), 4.38 (t, J = 6.7 Hz, |
| | | | | | 2H), 2.92-2.83 (m, 2H), |
| | | | | | 2.68-2.60 (m, 2H), 2.34 (s, |
| | | | | | 6H), 1.86 (h, J = 7.2 Hz, |
| | | | | | 2H), 1.07 (t, J = 7.4 Hz, |
| | | | | | 3H). |
| 521 | tert-butyl 3-[2- | OMe | ^{t}Bu | (4 min, | ¹H NMR (400 MHz, |
| | (dimethylamino)ethyl]- | | | basic): rt = | CDCl₃) δ 7.98 (s, 1H), 7.37 |
| | 5-methoxy-indole-1- | | | 2.10 min, | (s, 1H), 6.98 (d, J = 2.5 Hz, |
| | carboxylate | | | m/z = 305.1 | 1H), 6.91 (dd, J = 9.0, 2.5 |
| | | | | [M + H]⁺, | Hz, 1H), 3.86 (s, 3H), 2.87- |
| | | | | 100% purity. | 2.78 (m, 2H), 2.66-2.58 |
| | | | | | (m, 2H), 2.33 (s, 6H), 1.65 |
| | | | | | (s, 9H). |
| 522 | isopropyl 3-[2- | OMe | ⁱPr | (4 min, | ¹H NMR (400 MHz, |
| | (dimethylamino)ethyl]- | | | basic): rt = | CDCl₃) δ 8.02 (s, 1H), 7.40 |
| | 5-methoxy-indole-1- | | | 1.85 min, | (s, 1H), 6.99 (d, J = 2.5 Hz, |
| | carboxylate | | | m/z = 305.1 | 1H), 6.93 (dd, J = 8.9, 2.5 |
| | | | | [M + H]⁺, | Hz, 1H), 5.23 (hept, J = 6.2 |
| | | | | 100% purity. | Hz, 1H), 3.87 (s, 3H), 2.88- |
| | | | | | 2.79 (m, 2H), 2.67-2.58 |
| | | | | | (m, 2H), 2.34 (s, 6H), 1.44 |
| | | | | | (d, J = 6.2 Hz, 6H). |
| 523 | propyl 3-[2- | OMe | n-Pr | (4 min, | ¹H NMR (400 MHz, |
| | (dimethylamino)ethyl]- | | | basic): rt = | CDCl₃) δ 8.02 (s, 1H), 7.41 |
| | 5-methoxy-indole-1- | | | 2.00 min, | (s, 1H), 6.99 (d, J = 2.5 Hz, |
| | carboxylate | | | m/z =305.1 | 1H), 6.93 (dd, J = 8.9, 2.5 |
| | | | | [M + H]⁺, | Hz, 1H), 4.36 (t, J = 6.7 Hz, |
| | | | | 100% purity | 2H), 3.87 (s, 3H), 2.88- |
| | | | | | 2.79 (m, 2H), 2.67-2.58 |
| | | | | | (m, 2H), 2.33 (s, 6H), 1.85 |
| | | | | | (h, J = 7.2 Hz, 2H), 1.06 (t, |
| | | | | | J = 7.4 Hz, 3H). |
| 524 | isobutyl 3-[2- | H | ⁱBu | (4 min, | ¹H NMR (400 MHz, |
| | (dimethylamino)- | | | basic): rt = | CDCl₃) δ 8.06-8.00 (m, |
| | ethyl]indole-1- | | | 2.19 min, | 1H), 7.46-7.39 (m, 1H), |
| | carboxylateª | | | m/z = | 7.36-7.28 (m, 1H), 7.21 |
| | | | | 289.1.1 | (ddd, J = 8.3, 7.2, 1.4 Hz, |
| | | | | [M + H]⁺, | 1H), 7.17-7.09 (m, 1H), |
| | | | | 96% purity | 7.14 (s, 1H), 4.09 (d, J = 6.6 |
| | | | | | Hz, 2H), 2.80-2.71 (m, |
| | | | | | 2H), 2.56-2.48 (m, 2H), |
| | | | | | 2.22 (s, 6H), 2.02 (dh, J = |
| | | | | | 13.4, 6.7 Hz, 1H), 0.94 (d, J = |
| | | | | | 6.7 Hz, 6H). |

^{a}Following chromatography, further purification was performed using reverse phase chromatography: C18 (23 g cartridge) eluting with a gradient of MeCN (0.1% NEt₃/formic acid) (5% to 98%; v/v) in water (0.1% NEt₃/formic acid)

### Example 1-10: 1-[3-[2-(dimethylamino)ethyl]lindol-1-yl]ethenone (Compound 119)

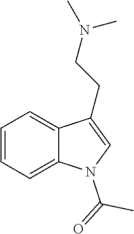 [SMILES: CC(=O)n1cc(CCN(C)C)c2ccccc21]

To a stirring solution of DMT (378 mg, 2.0 mmol) in THF (10 mL) at −78° C. was added NaHMDS, 1M solution in THF (3.0 mL, 3.0 mmol). The mixture was stirred at −78° C. for 15 min then AcCl (0.29 mL, 4.0 mmol) was added. The mixture allowed to warm up to rt and stirred overnight, then diluted with EtOAc (10 ml), washed with H₂O (10 mL), brine (10 mL), dried over Na₂SO₄, filtered and the filtrate was concentrated under reduced pressure. The residue was purified by column chromatography on silica gel (4 g cartridge) eluting with a gradient of MeOH in DCM (0-10%) to afford 1-[3-[2-(dimethylamino)ethyl]indol-1-yl]ethanone (Compound 119, 52 mg, 11% yield) as an oil. UPLC-MS analysis (4 min, basic): rt=1.51 min, m/z=231.1 [M+H]⁺; ¹H NMR (400 MHz, CDCl₃) δ 8.34 (d, J=8.0 Hz, 1H), 7.51-7.41 (m, 1H), 7.28 (ddd, J=8.3, 7.2, 1.4 Hz, 1H), 7.21 (td, J=7.5, 1.1 Hz, 1H), 7.19 (s, 1H), 2.85-2.76 (m, 2H), 2.58 (dd, J=8.9, 6.8 Hz, 2H), 2.54 (s, 3H), 2.27 (s, 6H).

The following compounds were made by analogous methods to that described for isobutyl-[3-[2-(dimethylamino)ethyl]indol-1-yl]ethanone (Compound 119)

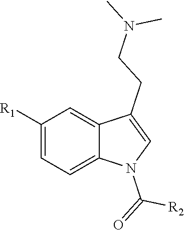

| Cpd | Name | R₁ | R₂ | UPLC-MS | 1H NMR |
|---|---|---|---|---|---|
| 122 | [3-[2- | H | Ph | (4 min, basic): rt = | ¹H NMR (400 MHz, CDCl₃) : δ |
| | (dimethylamino)ethyl]indol- | | | 2.01 min, m/z = | 8.36 (d, J = 8.1 Hz, 1H), 7.75- |
| | 1-yl]-phenyl-methanone | | | 293.1 [M + H]⁺, | 7.70 (m, 2H), 7.63-7.49 (m, |
| | | | | 100% purity. | 4H), 7.41-7.30 (m, 2H), 7.13 (d, |
| | | | | | J= 1.3 Hz, 1H), 2.86 (dd, J = 9.3, |
| | | | | | 6.5 Hz, 2H), 2.66-2.57 (m, 2H), |
| | | | | | 2.32 (s, 6H). |
| 120 | 1-[3-[2- | H | Et | (4 min, basic): rt = | ¹H NMR (400 MHz, DMSO-d₆) |
| | (dimethylamino)ethyl]indol- | | | 1.77 min, m/z = | δ 8.35 (d, J = 8.0 Hz, 1H), 7.70 |
| | 1-yl]propan-1-one | | | 245.1 [M + H]⁺, | (d, J = 1.2 Hz, 1H), 7.61 (dd, J = |
| | | | | 100% purity. | 7.3, 1.4 Hz, 1H), 7.36-7.22 (m, |
| | | | | | 2H), 3.02 (q, J = 7.3 Hz, 2H), |
| | | | | | 2.86-2.76 (m, 2H), 2.58 (dd, J = |
| | | | | | 8.6, 6.8 Hz, 2H), 2.23 (s, 6H), |
| | | | | | 1.18 (t, J = 7.3 Hz, 3H). |
| 108 | 1-[3-[2- | OMe | Et | (4 min, basic): rt = | |
| | (dimethylamino)ethyl]- | | | 1.66 min, m/z = | |
| | 5-methoxy-indol-1- | | | 275.1 [M + H]⁺, | |
| | yl]propan-1-one | | | 100% purity. | |
| 110 | [3-[2- | OMe | Ph | (4 min, basic): rt = | ¹H NMR (400 MHz, CDCl₃) δ |
| | (dimethylamino)ethyl]-5- | | | 1.93 min, m/z = | 8.26 (d, J = 9.0 Hz, 1H), 7.73- |
| | methoxy-indol-1- | | | 323.2 [M + H]⁺, | 7.67 (m, 2H), 7.62 (t, J = 7.4 Hz, |
| | yl]-phenyl- | | | 100% purity. | 1H), 7.54 (dd, J = 8.2, 6.7 Hz, |
| | methanone | | | | 2H), 7.18 (d, J = 2.4 Hz, 1H), |
| | | | | | 7.15 (s, 1H), 7.01 (dd, J = 9.0, 2.5 |
| | | | | | Hz, 1H), 3.93 (s, 3H), 3.23 (s, |
| | | | | | 2H), 3.11 (s, 2H), 2.76 (s, 6H). |
| 107 | 1-[3-[2- | OMe | Me | (4 min, basic): rt = | ¹H NMR (400 MHz, CDCl₃) δ |
| | (dimethylamino)ethyl]-5- | | | 1.48 min, m/z = | 8.27 (d, J = 9.0 Hz, 1H), 7.18- |
| | methoxy-indol-1- | | | 261.2 [M + H]⁺, | 7.08 (m, 1H), 6.95-6.86 (m, |
| | yl]ethanone | | | 96% purity. | 2H), 3.80 (s, 3H), 2.80 (q, J = 7.2 |
| | | | | | Hz, 2H), 2.61 (dd, J = 8.9, 6.7 Hz, |
| | | | | | 2H), 2.52 (s, 2H), 2.31 (d, J = 2.6 |
| | | | | | Hz, 6H). |

### Example 1-11: 3-[2-(dimethylamino)ethyl]-N,N-dimethyl-indole-1-carboxamide (Compound 525)

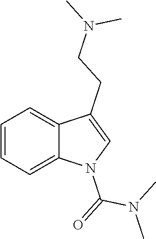 [SMILES: CN(C)CCc1cn(C(=O)N(C)C)c2ccccc12]

To a stirring solution of DMT (145 mg, 0.77 mmol) in THF (10 mL) at −78° C. was added NaHMDS, 1M solution in THF (1.2 mL, 1.2 mmol). The mixture was stirred at −78° C. for 15 min, then dimethyl carbamoyl chloride (166 mg, 1.54 mmol) was added. The mixture was allowed to warm to rt and stirred overnight, then diluted with EtOAc (10 mL), washed with H₂O (3×10 mL), dried over Na₂SO₄, filtered and the filtrate was concentrated under reduced pressure. The residue was purified by column chromatography on silica gel (4 g cartridge) eluting with a gradient of MeOH (0% to 10%; v/v) in DCM to afford 3-[2-(dimethylamino)ethyl]-N,N-dimethyl-indole-1-carboxamide (Compound 525, 59 mg, 30% yield) as an oil. UPLC-MS analysis (4 min, basic): rt=1.85 min, m/z=305.1 [M+H]⁺; ¹H NMR (400 MHz, CDCl₃) δ 7.61 (t, J=7.6 Hz, 2H), 7.32 (t, J=7.7 Hz, 1H), 7.28 (s, 1H), 7.26-7.21 (m, 1H), 3.34 (dd, J=10.3, 5.8 Hz, 2H), 3.22 (dd, J=10.2, 5.9 Hz, 2H), 3.09 (s, 6H), 2.81 (s, 6H).

A repeat experiment was additionally purified by reversed-phase chromatography, eluting with 0 to 100% acetonitrile in 0.1% formic acid. The pooled fractions were concentrated and lyophilised to give 3-(2-(dimethylamino)ethyl)-N,N-dimethyl-1H-indole-1-carboxamide formate (94 mg) as an oil. LC-MS (+ve mode): m/z=260.15 [M+H]⁺; ¹H NMR (300 MHz, CDCl₃) δ 8.43 (s, 1H, HCO), 7.53 (m, 2H, 2×ArH), 7.20 (m, 3H, 3×ArH), 3.11 (m, 4H, 2×CH₂), 3.02 (s, 6H, 2×NMe), 2.67 (s, 6H, 2×NMe); ¹³C NMR (75.5 MHz, CDCl₃) δ 167.7, 154.9, 135.8, 128.5, 142.1, 121.9, 118.7, 114.2, 113.8, 57.6, 43.0, 38.5, 21.0.

### Example 1-12: 3-(2-(dimethylamino)ethyl)-5-methoxy-N,N-dimethyl-1H-indole-1-carboxamide formate (Compound 526)

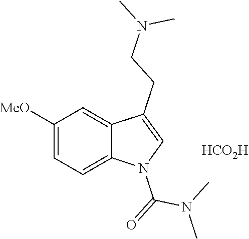 [SMILES: COc1ccc2c(c1)c(CCN(C)C)cn2C(=O)N(C)C ; "HCO2H"]

To a solution of 5-OMe-DMT (115 mg, 0.53 mmol) in THF (8 mL) at −78° C. under an atmosphere of N2 was added NaHMDS, 1M in THF (1.06 mL, 1.06 mmol) and the mixture was stirred for 30 min at −78° C., then dimethylcarbamyl chloride (110 mg, 97 μL, 1.06 mmol) was added. The mixture was stirred at −78° C. for 20 min, then warmed to rt and stirred overnight. H₂O (1 mL) was added and the mixture was concentrated under reduced pressure. The residue was purified by column chromatography on silica gel, eluting with 0 to 50% MeOH in EtOAc (containing 0.1% Et₃N), then purified further by reverse-phase HPLC, eluting with 0 to 100% acetonitrile in 0.1% formic acid to give 3-(2-(dimethylamino)ethyl)-5-methoxy-N,N-dimethyl-1H-indole-1-carboxamide formate (Compound 526, 118 mg, 66%) as an oil. LC-MS (+ve mode): m/z=290.15 [M+H]⁺; ¹H NMR (300 MHz, CDCl₃) δ 8.43 (s, 1H, formate), 7.51 (d, J=9.0 Hz, 1H, ArH), 7.12 (s, 1H, ArH), 7.06 (d, J=2.4 Hz, 1H, ArH), 6.87 (dd, J=9.0, 2.5 Hz, 1H, ArH), 3.82 (s, 3H, OMe), 3.13 (s, 4H, 2×CH₂), 3.07 (s, 6H, 2×NMe), 2.66 (s, 6H, 2×NMe); ¹³C NMR (75.5 MHz, CDCl₃) δ 167.7, 155.5, 155.0, 130.7, 129.3, 124.6, 114.6, 114.0, 113.3, 101.2, 57.4, 56.0, 43.0, 38.5, 21.1.

### Example 1-13: [1,4′-Bipiperidin]-1′-yl(3-(2-(dimethylamino)ethyl)-1H-indol-1-yl)methanone di-formate (di-formate salt of Compound 88)

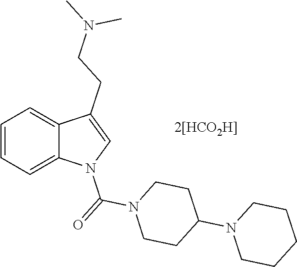 [SMILES: CN(C)CCc1cn(C(=O)N2CCC(N3CCCCC3)CC2)c2ccccc12]

To a solution of DMT (146 mg, 0.78 mmol) in THF (10 mL) at −78° C. under an atmosphere of N2 was added NaHMDS, 1M in THF (3.1 mL, 3.1 mmol) and the mixture was stirred for 30 min at −78° C. 1-Chlorocarbonyl-4-piperidinopiperidine hydrochloride (414 mg, 1.55 mmol) was added, and the mixture was stirred at −78° C. for 20 min then warmed to rt and stirred overnight. H₂O (2 mL) was added and the mixture was concentrated under reduced pressure. The residue was purified by reverse-phase chromatography, eluting with 0 to 100% acetonitrile in 0.1% formic acid to give [1,4′-bipiperidin]-1′-yl(3-(2-(dimethylamino)ethyl)-1H-indol-1-yl)methanone di-formate (di-formate salt of Compound 88, 255 mg, 69%) as a semi-solid. LC-MS (+ve mode): m/z=383.25 [M+H]⁺; ¹H NMR (300 MHz, DMSO-d₆) δ 8.26 (s, 2H, 2×formate), 7.62 (m, 2H, 2×ArH), 7.38 (s, 1H, ArH), 7.27 (m, 1H, ArH), 7.17 (m, 1H, ArH), 3.94 (m, 2H, CH₂), 3.60 (m, 1H, CH), 3.06 (t, J=12.5 Hz, 2H, CH₂), 2.89 (m, 2H, CH₂), 2.76 (m, 2H, CH₂), 2.61 (m, 6H, 3×CH₂), 2.38 (s, 6H, 2×NMe), 1.54 (br, 6H, 3×CH₂), 1.42 (br, 4H, 2×CH₂); ¹³C NMR (75.5 MHz, DMSO-d₆) δ 164.6, 153.5, 135.8, 129.4, 124.7, 123.8, 121.7, 119.5, 116.5, 113.7, 61.9, 58.6, 50.0, 46.0, 44.7, 27.8, 26.0, 24.5, 22.2.

### Example 1-14: [1,4′-bipiperidin]-1′-yl(3-(2-(dimethylamino)ethyl)-5-methoxy-1H-indol-1-yl)methanone di-formate (di-formate salt of Compound 96)

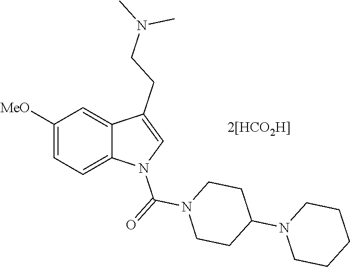 [SMILES: COc1ccc2c(c1)c(CCN(C)C)cn2C(=O)N1CCC(N2CCCCC2)CC1]

To a mixture of 5-methoxy-N,N-dimethyltryptamine (169 mg, 0.78 mmol) in THF (10 mL) at −78° C. under an atmosphere of N2 was added NaHMDS, 1M in THF (3.1 mL, 3.1 mmol) and the mixture was stirred for 30 min at −78° C. 1-Chlorocarbonyl-4-piperidinopiperidine HCl (414 mg, 1.55 mmol) was added, the mixture was stirred at −78° C. for 20 min, then warmed to rt and stirred overnight. H₂O (2 mL) was added and the mixture was concentrated under reduced pressure. The residue was purified by reversed-phase chromatography, eluting with 0 to 100% acetonitrile in 0.1% formic acid to give [1,4′-bipiperidin]-1′-yl(3-(2-(dimethylamino)ethyl)-5-methoxy-1H-indol-1-yl)methanone di-formate (di-formate salt of Compound 96, 311 mg, 62%) as a semi-solid. LC-MS (+ve mode): m/z=413.30 [M+H]⁺; ¹H NMR (300 MHz, DMSO-d₆) δ 8.25 (s, 2H, 2×formate), 7.51 (d, J=8.9 Hz, 1H, ArH), 7.33 (s, 1H, ArH), 7.10 (d, J=2.5 Hz, 1H, ArH), 6.88 (dd, J=8.9, 2.5 Hz, 1H, ArH), 3.92 (m, 2H, CH₂), 3.80 (s, 3H, OMe), 3.02 (t, J=12.5 Hz, 2H, CH₂), 2.87 (m, 2H, CH₂), 2.78 (m, 2H, CH₂), 2.63 (m, 4H, 2×CH₂), 2.40 (s, 6H, 2×NMe), 1.82 (d, J=12.6 Hz, 2H, CH₂), 1.47 (m, 8H, 4×CH₂); ¹³C NMR (75.5 MHz, DMSO-d₆) δ 164.6, 155.2, 153.7, 130.6, 130.1, 125.2, 116.3, 114.6, 113.0, 102.0, 61.9, 58.3, 55.9, 50.0, 46.0, 44.5, 27.8, 25.9, 25.0, 22.1.

### Example 1-15: 2-(4-(3-(2-(dimethylamino)ethyl)-1H-indol-1-yl)-2-methyl-4-oxobutan-2-yl)-3,5-dimethylphenyl acetate (Compound 413)

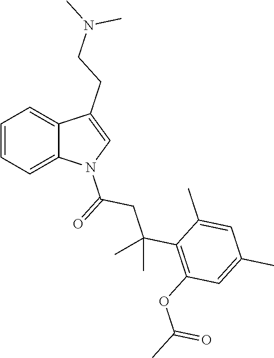 [SMILES: CC(=O)Oc1cc(C)cc(C)c1C(C)(C)CC(=O)n1cc(CCN(C)C)c2ccccc21]

### Step 1: 2-(4-Chloro-2-methyl-4-oxobutan-2-yl)-3,5-dimethylphenyl acetate

To a mixture of 3-(2-acetoxy-4,6-dimethylphenyl)-3-methylbutyric acid (0.56 g, 2.12 mmol) in DCM (2.1 mL) at 0° C. under an atmosphere of N2 was added oxalyl chloride (268 mg, 0.18 mL, 2.12 mmol). The mixture was warmed to rt and stirred for 2 h 45 min, then concentrated under reduced pressure to give 2-(4-chloro-2-methyl-4-oxobutan-2-yl)-3,5-dimethylphenyl acetate as an oil, which was used directly in the next step.

### Step 2: 2-(4-(3-(2-(dimethylamino)ethyl)-1H-indol-1-yl)-2-methyl-4-oxobutan-2-yl)-3,5-dimethylphenyl acetate

To a solution of DMT (100 mg, 0.53 mmol) in THF (10 mL) at −78° C. under an atmosphere of N2 was added NaHMDS, 1M in THF (1.06 mL, 1.06 mmol) and the mixture was stirred for 30 min at −78° C. 2-(4-Chloro-2-methyl-4-oxobutan-2-yl)-3,5-dimethylphenyl acetate solution, 1M in THF (1.06 mL, 1.06 mmol) was added and the mixture was stirred at −78° C. for 20 min, then warmed to rt and stirred overnight. H₂O (2 mL) was added and the mixture was concentrated under reduced pressure. The residue was purified by reversed-phase HPLC, eluting with 0 to 100% acetonitrile in 0.1% formic acid to give the product (56 mg). A further batch was prepared on the same scale to afford 79 mg of material.

The combined materials from batches 1 and 2 (135 mg) were purified by column 1) chromatography on silica gel (MeOH/EtOAc (containing 0.1% triethylamine), 0:1 to 1) to afford 2-(4-(3-(2-(dimethylamino)ethyl)-1H-indol-1-yl)-2-methyl-4-oxobutan-2-yl)-3,5-dimethylphenyl acetate (Compound 413, 67 mg, 15% based on the two batches) as an oil. LC-MS (+ve mode): m/z=435.25 [M+H]⁺; ¹H NMR (300 MHz, CDCl₃) δ 8.41 (d, J=7.8 Hz, 1H, ArH), 7.50 (m, 1H, ArH), 7.28 (m, 3H, 3×ArH), 6.83 (d, J=2.1 Hz, 1H, ArH), 6.55 (d, J=2.0 Hz, 1H, ArH), 3.41 (s, 2H, CH₂), 2.91 (m, 2H, CH₂), 2.69 (m, 2H, CH₂), 2.55 (s, 3H, COMe), 2.41 (s, 6H, 2×NMe), 2.24 (s, 3H, ArMe), 2.19 (s, 3H, ArMe), 1.67 (s, 6H, 2×CMe); ¹³C NMR (75.5 MHz, CDCl₃) δ 170.0, 169.3, 149.2, 138.0, 136.3, 136.0, 133.9, 132.7, 130.3, 125.1, 123.3, 123.1, 122.0, 119.6, 118.6, 117.0, 60.4, 58.9, 45.1, 39.2, 31.6, 25.6, 23.0, 21.8, 20.3, 17.7.

### Example 1-16: 2-(4-(3-(2-(dimethylamino)ethyl)-5-methoxy-1H-indol-1-yl)-2-methyl-4-oxobutan-2-yl)-3,5-dimethylphenyl (Compound 405)

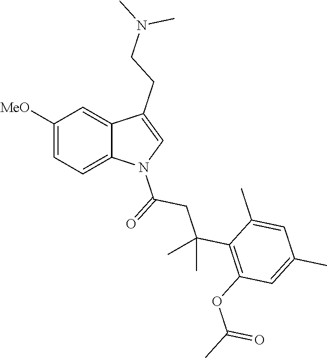 [SMILES: COc1ccc2c(c1)c(CCN(C)C)cn2C(=O)CC(C)(C)c1c(C)cc(C)cc1OC(C)=O]

Compound 405 was prepared using the procedure similar to that in Example 1-15 for Compound 413, afforded as a semi-solid (71 mg, 14% yield). LC-MS (+ve mode): m/z=465.25 [M+H]⁺; ¹H NMR (300 MHz, CDCl₃) δ 8.21 (d, J=9.0 Hz, 1H, ArH), 7.19 (s, 1H, ArH), 6.96 (d, J=2.4 Hz, 1H, ArH), 6.90 (dd, J=9.0, 2.4 Hz, 1H, ArH), 6.81 (br. s, 1H, ArH), 6.56 (br. s, 1H, ArH), 3.86 (s, 3H, OMe), 3.37 (s, 2H, CH₂), 2.86 (m, 2H, CH₂), 2.66 (m, 2H, CH₂), 2.53 (s, 3H, COMe), 2.40 (s, 6H, 2×NMe), 2.22 (s, 3H, ArMe), 2.20 (s, 3H, ArMe), 1.67 (s, 6H, 2×CMe); ¹³C NMR (75.5 MHz, CDCl₃) δ 170.0, 169.0, 156.3, 149.3, 138.0, 136.3, 133.9, 132.7, 130.7, 123.1, 122.6, 113.0, 101.8, 58.8, 55.8, 45.1, 39.2, 31.6, 25.6, 21.8, 20.3.

### Example 1-17: 2-Methoxyethyl 3-(2-(dimethylamino)ethyl)-1H-indole-1-carboxylate formate (Compound 25)

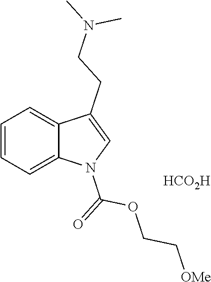 [SMILES: COCCOC(=O)n1cc(CCN(C)C)c2ccccc21 ; "HCO2H"]

To a solution of DMT (100 mg, 0.53 mmol) in THF (8 mL) at −78° C. under an atmosphere of N2 was added NaHMDS, 1M in THF (1.06 mL, 1.06 mmol) and the mixture was stirred for 30 min at −78° C. 2-Methoxyethyl chloroformate (147 mg, 123 μL, 1.06 mmol) was added, the mixture was stirred at −78° C. for 20 min, then allowed to warm to rt and stirred for 2 h. H₂O (2 mL) was added and the mixture was concentrated under reduced pressure. The residue was purified by reversed-phase HPLC, eluting with 0 to 100% acetonitrile in 0.1% formic acid to give the product (45 mg). A further batch was prepared on the same scale to afford 40 mg of material.

The materials from batches 1 and 2 (85 mg) were combined to afford 2-methoxyethyl 3-(2-(dimethylamino)ethyl)-1H-indole-1-carboxylate formate (Compound 25, 81 mg, 23%) as an oil. LC-MS (+ve mode): m/z=291.15 [M+H]⁺; ¹H NMR (300 MHz CDCl₃) δ 8.49 (s, 1H, HCO), 8.18 (d, J=8.1 Hz, 1H, ArH), 7.57 (m, 1H, ArH), 7.49 (s, 1H, ArH), 7.35 (m, 1H, ArH), 7.30 (m, 1H, ArH), 4.56 (m, 2H, CH₂), 3.76 (m, 2H, CH₂), 3.42 (s, 3H, OMe), 3.09 (m, 4H, 2×CH₂), 2.68 (s, 6H, 2×NMe); ¹³C NMR (75.5 MHz, CDCl₃) δ 167.7, 129.8, 125.1, 123.1, 122.9, 118.8, 117.1, 115.5, 70.3, 65.9, 59.1, 57.4, 43.2, 21.2.

### Example 1-18: 2-Methoxyethyl 3-(2-(dimethylamino)ethyl)-5-methoxy-1H-indole-1-carboxylate formate (Compound 22)

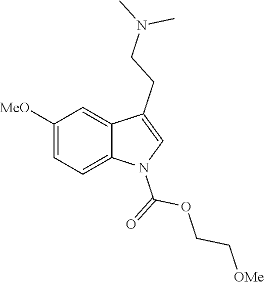 [SMILES: COCCOC(=O)n1cc(CCN(C)C)c2cc(OC)ccc21]

Compound 22 was prepared using the procedure similar to that in Example 1-17 for Compound 25. The materials from batches 1 and 2 (127 mg) were combined and the resultant material was purified by column chromatography on silica gel (MeOH/EtOAc (containing 0.1% triethylamine), 0:1 to 1:1) to afford 2-methoxyethyl 3-(2-(dimethylamino)ethyl)-5-methoxy-1H-indole-1-carboxylate (Compound 22, 51 mg, 15%) as a semi-solid. LC-MS (+ve mode): m/z=321.10 [M+H]⁺; ¹H NMR (300 MHz, CDCl₃) δ 8.04 (d, J=9.0 Hz, 1H, ArH), 7.42 (s, 1H, ArH), 7.00 (d, J=2.4 Hz, 1H, ArH), 6.94 (dd, J=9.0, 2.5 Hz, 1H, ArH), 4.54 (m, 2H, CH₂), 3.87 (s, 3H, ArOMe), 3.75 (m, 2H, CH₂), 3.44 (s, 3H, OMe), 2.87 (m, 2H, CH₂), 2.67 (m, 2H, CH₂), 2.38 (s, 6H, 2×NMe); ¹³C NMR (75.5 MHz, CDCl₃) δ 158.7, 156.1, 131.5, 123.0, 123.0, 119.3, 116.1, 113.0, 102.0, 70.4, 65.7, 59.0, 59.0, 55.8, 45.3, 23.3.

### Example 1-19: 4-(3-(2-(dimethylamino)ethyl)-1H-indol-1-yl)-4-oxobutanoic acid formate salt (Compound 529)

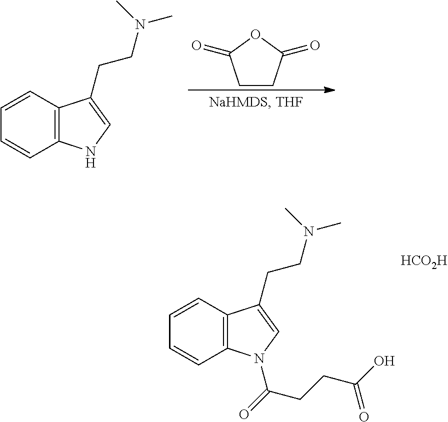

NaHMDS, 1M in THF (1.06 mL, 0.56 mmol) was added to a mixture of DMT (100 mg, 0.53 mmol) in THF (5 mL) at −78° C. and the mixture was stirred for 30 min. Succinic anhydride (106 mg, 1.06 mmol) was added and the resulting mixture was stirred at −78° C. for 30 min, then allowed to warm to rt and stirred for 16 h. H₂O (1 mL) was added and the mixture was concentrated under reduced pressure. The residue was purified using preparative HPLC using H₂O (0.1% formic acid) in 30% acetonitrile to afford Compound 529 (50 mg, 32%) as a solid. LC-MS (+ve mode): m/z=289.10 [M+H]⁺; ¹H NMR (300 MHz, CD₃OD) δ 8.42 (s, 1H, HCO), 8.37 (m, 1H, ArH), 7.62 (m, 1H, ArH), 7.30 (m, 2H, 2×ArH), 3.45 (m, 2H, CH₂), 3.15 (m, 2H, CH₂), 2.93 (s, 6H, 2×NMe), 2.71 (t, J=6.8 Hz, 2H, CH₂); ¹³C NMR (75.5 MHz, CD₃OD) δ 171.2, 154.0, 129.6, 125.0, 123.3, 123.3, 118.3, 116.3, 116.2, 56.6, 42.2, 30.9, 29.4, 20.1.

### Example 1-20: 4-(3-(2-(dimethylamino)ethyl)-5-methoxy-1H-indol-1-yl)-4-oxobutanoic acid (Compound 530)

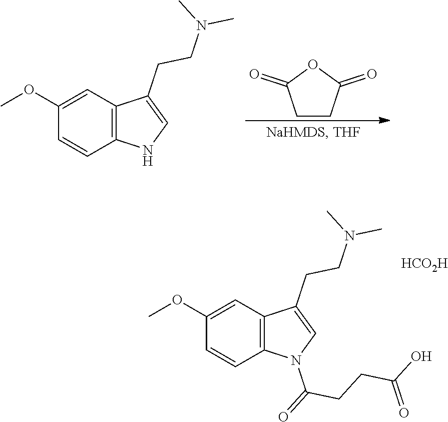

Compound 530 was prepared using the procedure similar to that in Example 1-19 for Compound 529, afforded as a solid (121 mg, 76%). LC-MS (+ve mode): m/z=319.10 [M+H]⁺; ¹H NMR (300 MHz, DMSO-d₆) δ 8.20 (m, 2H, HCO and ArH) 7.73 (s, 1H, ArH), 7.11 (d, J=2.8 Hz, 1H, ArH), 6.92 (dd, J=8.8, 2.8 Hz, 1H, ArH), 3.82 (s, 3H, OMe), 3.19 (m, 2H, CH₂) 2.83 (m, 2H, CH₂), 2.65 (m, 4H, 2×CH₂), 2.30 (s, 6H, 2×NMe).

### Example 1-21: 5-(3-(2-(dimethylamino)ethyl)-1H-indol-1-yl)-5-oxopentanoic acid (Compound 531)

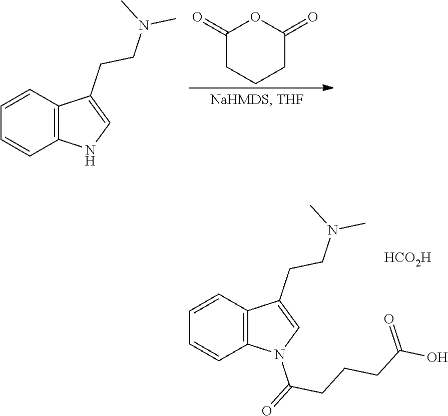

Compound 531 was prepared using the procedure similar to that in Example 1-19 for Compound 529, afforded as an oil (53 mg, 29%). LC-MS (+ve mode): m/z=303.10 [M+H]⁺; ¹H NMR (300 MHz, CD₃OD) δ 8.43 (dd, J=6.9, 1.4 Hz, 1H ArH), 8.39 (s, 1H, HCO), 7.77 (s, 1H, ArH), 7.66 (dd, J=6.9, 2.0, 1H, ArH), 7.35 (m, 2H, 2×ArH), 3.51 (m, 2H, CH₂), 3.22 (m, 2H, CH₂), 3.06 (m, 2H, CH₂), 2.97 (s, 6H, 2×NMe), 2.47 (m, 2H, CH₂), 2.10 (m, 2H, CH₂); ¹³C NMR (75.5 MHz, CD₃OD) δ 176.4, 171.6, 166.6, 136.0, 129.6, 125.1, 123.4, 123.3, 118.2, 116.3, 106.3, 56.6, 42.1, 34.4, 33.1, 20.1, 20.1.

### Example 1-22: 5-(3-(2-(dimethylamino)ethyl)-5-methoxy-1H-indol-1-yl)-5-oxopentanoic acid (Compound 532)

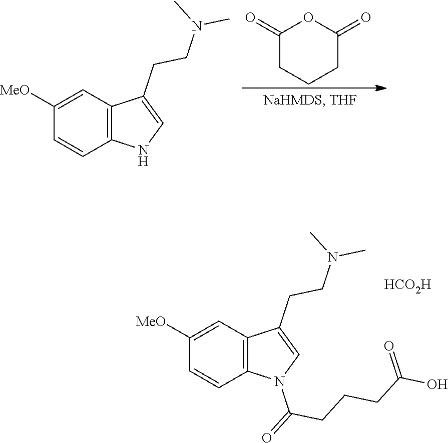

Compound 532 was prepared using the procedure similar to that in Example 1-19 for Compound 529, afforded as an oil (64 mg, 36%). LC-MS (+ve mode): m/z=333.10 [M+H]⁺; ¹H NMR (300 MHz, DMSO-d₆) δ 8.24 (s, 1H, HCO), 8.20 (br, 1H, ArH), 7.69 (s, 1H, ArH), 7.12 (d, J=2.4 Hz, 1H, ArH), 6.92 (dd, J=9.0, 2.4 Hz, 1H, ArH), 3.80 (s, 3H, OMe), 2.99 (t, J=7.2 Hz, 2H, CH₂), 2.83 (m, 2H, CH₂), 2.73 (m, 2H, CH₂), 2.36 (m, 7H, 2×NMe+CH₂), 1.90 (m, 2H, CH₂).

### Example 1-23: (Pivaloyloxy)methyl 3-(2-(dimethylamino)ethyl)-5-methoxy-1H-indole-1-carboxylate (Compound 369)

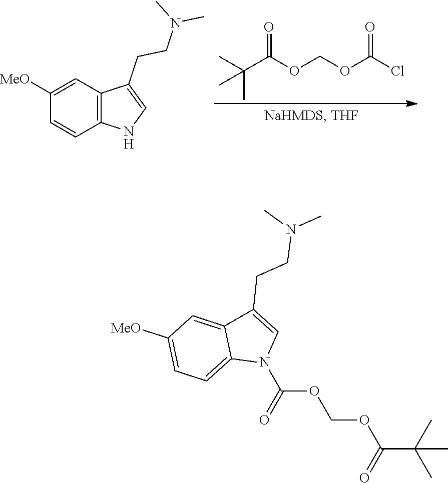

NaHMDS, 1M in THF (1.33 mL, 1.33 mmol) was added to a solution of 5-OMe-DMT (145 mg, 0.67 mmol) in THF (5 mL) at −78° C. and stirred for 30 min. Chlorocarbonyl-oxy-methyl, 2,2 dimethylpropanoate (129 mg, 0.67 mmol) was added and the resulting mixture was stirred at −78° C. for 30 min, then allowed to warm to rt and stirred for 16 h. H₂O (1 mL) was added and the mixture was concentrated under reduced pressure. The residue was purified by column chromatography on silica gel eluting (MeOH (0.1% Et₃N)/EtOAc (0.1% Et₃N), followed by preparative-HPLC using a gradient of H₂O in acetonitrile to afford Compound 369 (46 mg, 17%) as a solid. LC-MS (+ve mode): m/z=377.20 [M+H]⁺; ¹H NMR (300 MHz, CD₃OD) δ 7.87 (br. s, 1H, ArH), 7.33 (s, 1H, ArH), 6.98 (d, J=2.5 Hz, 1H, ArH), 6.84 (dd, J=8.8, 2.5 Hz, 1H, ArH), 5.92 (s, 2H, CH₂), 3.75 (s, 3H, OMe), 2.78 (m, 2H, CH₂), 2.78 (m, 2H, CH₂), 2.26 (s, 6H, 2×NMe), 1.12 (s, 9H, ^{t}Bu); ¹³C NMR (75.5 MHz, CD₃OD) δ 177.0, 131.5, 122.5, 120.0, 115.5, 112.9, 101.7, 80.9, 58.3, 54.7, 43.9, 38.4, 29.4, 25.8, 22.3.

### Example 1-24: (Pivaloyloxy)methyl 3-(2-(dimethylamino)ethyl)-1H-indole-1-carboxylate diformate (Compound 337)

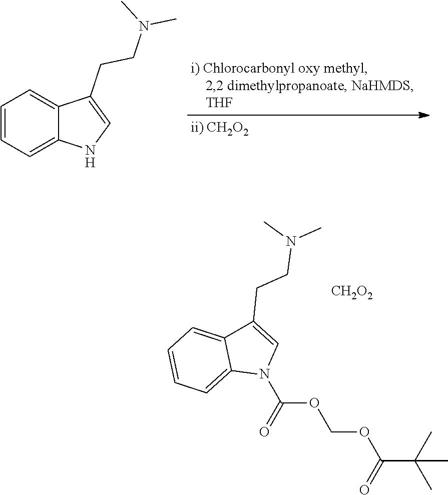

NaHMDS, 1M in THF (5.5 mL, 5.5 mmol) was added to a solution of DMT (0.52 g, 2.75 mmol) in anhydrous THF (40 mL) at −78° C. and stirred for 30 min. Chlorocarbonyl oxy methyl, 2,2 dimethylpropanoate (0.54 g, 2.75 mmol) was added and the mixture was stirred at −78° C. for 30 min, then allowed to warm to rt and stirred for 16 h. The mixture was concentrated to a semi-solid, which was purified using column chromatography on silica gel eluting with a gradient of MeOH (0.1% Et₃N) in EtOAc (0.1% Et₃N) followed by reversed-phase chromatography using a gradient of H₂O (formic acid 0.1%) in acetonitrile to afford Compound 337 (211 mg, 19%) as a solid. LC-MS (+ve mode): m/z=347.15 [M+H]⁺; ¹H NMR (300 MHz, CD₃OD) δ 8.44 (s, 1H, HCO₂H), 8.13 (d, J=7.8 Hz, 1H, ArH), 7.65 (m, 2H, 2×ArH), 7.36 (m, 2H, 2×ArH), 6.05 (s, 2H, CH₂), 3.43 (m, 2H, CH₂), 3.17 (m, 2H, CH₂), 2.92 (s, 6H, 2×NMe), 1.12 (s, 9H, ^{t}Bu); ¹³C NMR (75 MHz, CD₃OD) δ 177.0, 167.7, 135.6, 129.8, 125.0, 123.3, 123.0, 118.7, 116.8, 115.0, 81.0, 56.5, 42.1, 38.4, 25.8, 20.0.

### Example 1-25: Methyl 4-(3-(2-(dimethylamino)ethyl)-1H-indol-1-yl)-4-oxobutanoate (Compound 533)

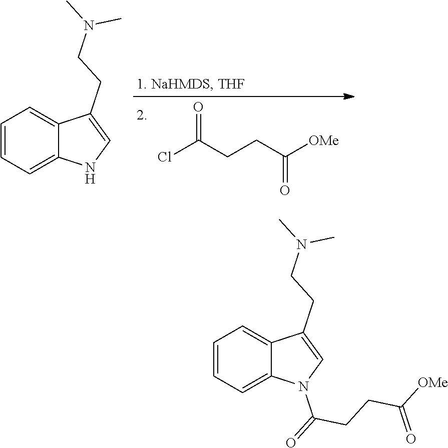

NaHMDS, 1M in THF (1.12 mL, 1.12 mmol) was added to a stirred solution of DMT (200 mg, 1.06 mmol) in THF (5 mL) at −78° C. After 30 min the resulting mixture was added dropwise to O-methyl succinyl chloride [CAS No: 1490-25-1] (163 mg, 1.08 mmol) and the mixture was stirred at rt for 16 h. EtOAc (30 mL) was added and the mixture was washed with saturated aqueous NaHCO₃ (50 mL) and brine (50 mL). The organic layer was dried (MgSO₄), filtered and the filtrate was concentrated under reduced pressure. The crude residue was purified by column chromatography on silica gel (eluting with a gradient of MeOH in CH₂Cl₂) to give methyl 4-(3-(2-(dimethylamino)ethyl)-1H-indol-1-yl)-4-oxobutanoate (100 mg, 34%) as a solid. LC-MS (+ve mode): m/z=303.10 [M+H]⁺; ¹H NMR (300 MHz, CDCl₃) δ 8.41 (d, J=7.8 Hz, 1H, ArH), 7.54 (d, J=7.8 Hz, 1H, ArH), 7.33 (m, 3H, 3×ArH), 3.73 (s, 3H, OMe), 3.25 (t, J=9.3 Hz, 2H, CH₂), 2.87 (m, 4H, 2×CH₂), 2.65 (m, 2H, CH₂), 2.35 (s, 6H, 2×NMe); ¹³C NMR (75.5 MHz, CDCl₃) δ 173.0, 169.5, 130.7, 129.2, 125.4, 125.4, 123.6, 121.3, 119.0, 116.8, 59.3, 52.2, 45.6, 30.8, 28.5, 23.6.

### Example 1-26: Methyl 4-(3-(2-(dimethylamino)ethyl)-5-methoxy-1H-indol-1-yl)-4-oxobutanoate (Compound 534)

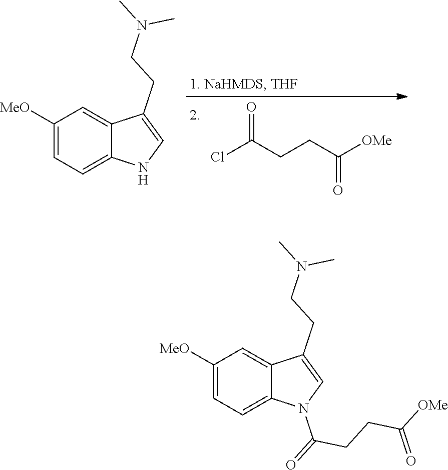 [SMILES: COc1ccc2[nH]cc(CCN(C)C)c2c1 ; COC(=O)CCC(=O)Cl ; COC(=O)CCC(=O)n1cc(CCN(C)C)c2cc(OC)ccc21]

Compound 534 was prepared using the procedure similar to that in Example 1-25 for Compound 533, afforded as an oil (54 mg, 31%). LC-MS (+ve mode): m/z=333.15 [M+H]⁺; ¹H NMR (300 MHz, CDCl₃) δ 8.28 (d, J=8.7 Hz, 1H, ArH), 7.27 (s, 1H, ArH), 6.93 (m, 2H, 2×ArH), 3.84 (s, 3H, OMe), 3.71 (s, 3H, OMe), 3.19 (t, J=6.9 Hz, 2H, CH₂), 2.81 (m, 4H, 2×CH₂), 2.62 (m, 2H, CH₂), 2.32 (s, 6H, 2×NMe); ¹³C NMR (75.5 MHz, CDCl₃) δ 173.0, 169.1, 156.5, 131.7, 130.7, 121.9, 121.1, 117.5, 113.2, 102.1, 59.1, 55.8, 52.1, 45.5, 30.5, 28.5, 23.6.

### Example 1-27: (S)-di-tert-butyl (6-(3-(2-(dimethylamino)ethyl)-1H-indol-1-yl)-6-oxohexane-1,5-diyl)dicarbamate (Compound 535)

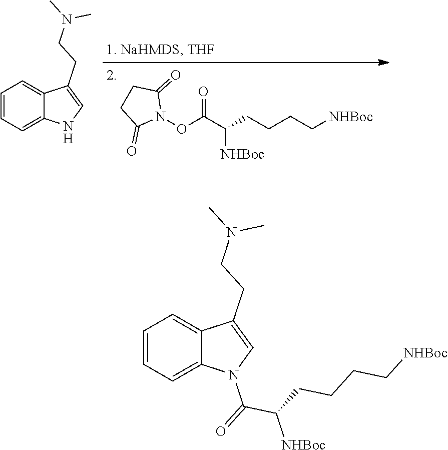 [SMILES: CN(C)CCc1c[nH]c2ccccc12 ; CC(C)(C)OC(=O)NCCCC[C@H](NC(=O)OC(C)(C)C)C(=O)ON1C(=O)CCC1=O ; CN(C)CCc1cn(C(=O)[C@H](CCCCNC(=O)OC(C)(C)C)NC(=O)OC(C)(C)C)c2ccccc12]

NaHMDS, 1M in THF (1.67 mL, 1.67 mmol) was added to a stirred solution of DMT (300 mg, 1.59 mmol) in THF (5 mL) at −78° C. After 30 min, a solution of Boc-lysine-(Boc)-O-succinimide [CAS No: 30189-36-7] (0.67 g, 1.51 mmol) in THF (5 mL) was added and the mixture was stirred at rt for 16 h. The mixture was concentrated under reduced pressure and the residue was purified by column chromatography on silica gel (eluting with a gradient of MeOH/EtOAc) to give (S)-di-tert-butyl (6-(3-(2-(dimethylamino)ethyl)-1H-indol-1-yl)-6-oxohexane-1,5-diyl)dicarbamate (186 mg, 23%) as a solid. LC-MS (+ve mode): m/z=517.35 [M+H]⁺; ¹H NMR (300 MHz, CDCl₃) δ 8.44 (d, J=8.1 Hz, 1H, ArH), 7.54 (dd, J=7.5, 1.8 Hz, 1H, ArH), 7.35 (m, 3H, 3×ArH), 5.46 (d, J=9.0 Hz, 1H, NH), 5.05 (m, 1H, CH), 4.62 (br. s, 1H, NH), 3.09 (m, 2H, CH₂), 2.90 (t, J=7.5 Hz, 2H, CH₂), 2.67 (t, J=7.2 Hz, 2H, CH₂), 2.36 (s, 6H, 2×NMe), 1.93 (m, 2H, CH₂), 1.75 (m, 2H, CH₂), 1.55 (m, 2H, CH₂), 1.46 (s, 9H, ^{t}Bu), 1.42 (s, 9H, ^{t}Bu); ¹³C NMR (75.5 MHz, CDCl₃) δ 170.8, 156.2, 155.7, 136.2, 131.0, 125.7, 124.1, 122.0, 121.2, 119.1, 117.0, 80.4, 77.4, 59.0, 52.8, 45.5, 40.3, 33.4, 28.6, 28.5, 23.5, 22.6.

### Example 1-28: (S)-di-tert-butyl (6-(3-(2-(dimethylamino)ethyl)-5-methoxy-1H-indol-1-yl)-6-oxohexane-1,5-diyl)dicarbamate (Compound 536)

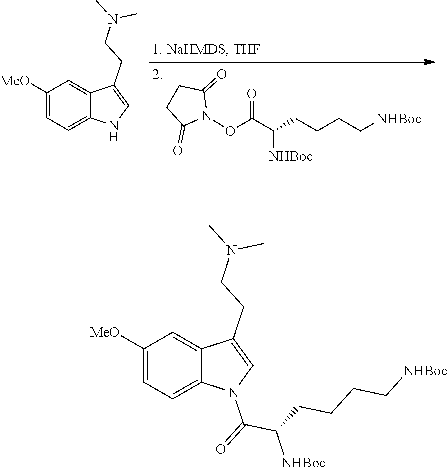 [SMILES: COc1ccc2[nH]cc(CCN(C)C)c2c1 ; CC(C)(C)OC(=O)NCCCC[C@H](NC(=O)OC(C)(C)C)C(=O)ON1C(=O)CCC1=O ; COc1ccc2c(c1)c(CCN(C)C)cn2C(=O)[C@H](CCCCNC(=O)OC(C)(C)C)NC(=O)OC(C)(C)C]

Compound 536 was prepared according to a procedure analogous to that provided in Example 1-27 for Compound 536, and was obtained as a solid (234 mg, 43%). LC-MS (+ve mode): m/z=547.35 [M+H]⁺; ¹H NMR (300 MHz, CDCl₃) δ 8.32 (d, J=8.7 Hz, 1H, ArH), 7.35 (s, 1H, ArH), 6.97 (m, 2H, 2×ArH), 5.43 (d, J=8.7 Hz, 1H, NH), 5.01 (m, 1H, CH), 4.62 (br. s, 1H, NH), 3.87 (s, 3H, OMe), 3.10 (br. s, 2H, CH₂), 2.87 (t, J=7.2 Hz, 2H, CH₂), 2.66 (t, J=7.2 Hz, 2H, CH₂), 2.37 (s, 6H, 2×NMe, 1.91 (m, 2H, CH₂), 1.74 (m, 2H, CH₂), 1.50 (m, 2H, CH₂), 1.45 (s, 9H, ^{t}Bu), 1.42 (s, 9H, ^{t}Bu).

### Example 1-29: (S)-2,6-diamino-1-(3-(2-(dimethylamino)ethyl)-1H-indol-1-yl)hexan-1-one trihydrochloride (Compound 537)

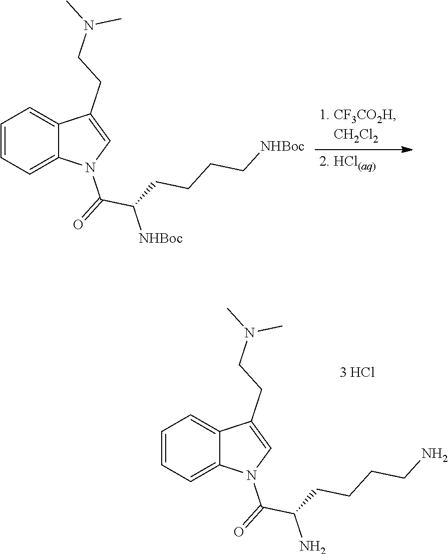 [SMILES: CN(C)CCc1cn(C(=O)[C@H](CCCCNC(=O)OC(C)(C)C)NC(=O)OC(C)(C)C)c2ccccc12 ; CN(C)CCc1cn(C(=O)[C@@H](N)CCCCN)c2ccccc12]

TFA (2.05 g, 1.38 mL, 18 mmol was added to a solution of (S)-di-tert-butyl (6-(3-(2-(dimethylamino)ethyl)-1H-indol-1-yl)-6-oxohexane-1,5-diyl)dicarbamate (Compound 535, 186 mg, 0.36 mmol) in DCM (5 mL) and the mixture was stirred at rt for 1 h. The mixture was concentrated under reduced pressure and the residue was azeotroped with CHCl₃ (4×10 mL) and MeOH (10 mL). The residue was dissolved in 1M HCl (2 mL, 2 mmol) and the resulting hydrochloride was purified by reversed-phase chromatography on silica eluting with a gradient of MeCN in 0.02% HCl_{(aq.)} to afford (S)-2,6-diamino-1-(3-(2-(dimethylamino)ethyl)-1H-indol-1-yl)hexan-1-one trihydrochloride (Compound 537, 102 mg, 67%) a solid. ESI MS: m/z=317.20 consistent for protonated parent ion of free-base [M+H]⁺; ¹H NMR (300 MHz, CD₃OD) δ 8.42 (dd, J=6.3, 2.4 Hz, 1H, ArH), 7.88 (s, 1H, ArH), 7.70 (dd, J=6.3, 2.1 Hz, 1H, ArH), 7.38 (m, 2H, 2×ArH), 5.04 (m, 1H, CH), 3.56 (m, 2H, CH₂), 3.24 (m, 2H, CH₂), 2.99 (s, 6H, 2×NMe), 2.90 (t, J=7.5 Hz, 2H, CH₂), 2.10 (m, 2H, CH₂), 1.70 (m, 2H, CH₂), 1.53 (m, 2H, CH₂); ¹³C NMR (75.5 MHz, CD₃OD) δ 169.0, 137.4, 131.3, 127.2, 125.9, 123.9, 120.3, 120.2, 117.8, 57.7, 54.0, 43.6, 40.1, 32.1, 28.1, 22.6, 21.4.

### Example 1-30: (S)-2,6-diamino-1-(3-(2-(dimethylamino)ethyl)-5-methoxy-1H-indol-1-yl)hexan-1-one trihydrochloride (Compound 538)

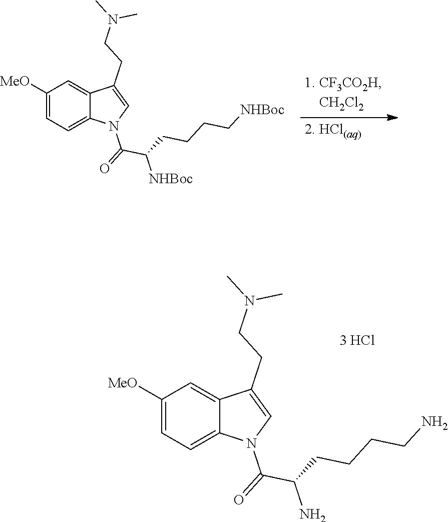 [SMILES: COc1ccc2c(c1)c(CCN(C)C)cn2C(=O)[C@H](CCCCNC(=O)OC(C)(C)C)NC(=O)OC(C)(C)C ; COc1ccc2c(c1)c(CCN(C)C)cn2C(=O)[C@@H](N)CCCCN]

Compound 538 was prepared using the procedure similar to that in Example 1-29 for Compound 537, afforded as a solid (142 mg, 72%). ESI MS: m/z=347.25 consistent for protonated parent ion of free-base [M+H]⁺; ¹H NMR (300 MHz, CD₃OD) δ 8.32 (d, J=9.0 Hz, 1H, ArH), 7.85 (s, 1H, ArH), 7.20 (d, J=2.7 Hz, 1H, ArH), 7.00 (dd, J=9.3, 2.7 Hz, 1H, ArH), 5.01 (m, 1H, CH), 3.88 (s, 3H, OMe), 3.55 (m, 2H, CH₂), 3.22 (m, 2H, CH₂), 3.00 (s, 6H, 2×NMe), 2.91 (m, 2H, CH₂), 2.09 (m, 2H, CH₂), 1.70 (m, 2H, CH₂), 1.57 (m, 2H, CH₂); ¹³C NMR (75.5 MHz, CD₃OD) δ 168.4, 159.0, 132.5, 131.7, 124.4, 120.3, 118.6, 115.2, 103.3, 57.7, 56.2, 53.8, 43.6, 40.1, 32.2, 28.2, 22.6, 21.4.

### Example 1-31: (S)-tert-butyl (1-(3-(2-(dimethylamino)ethyl)-1H-indol-1-yl)-1-oxopropan-2-yl)carbamate (Compound 539)

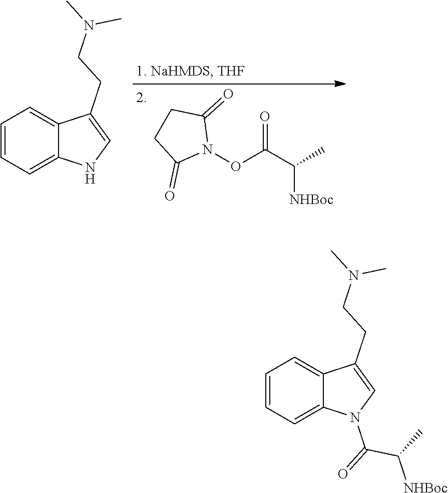

NaHMDS, 1M in THF (1.11 mL, 1.11 mmol) was added to a stirred solution of DMT (200 mg, 1.06 mmol) in anhydrous THF (5 mL) at −78° C. After 30 min, Boc-alanine-O-succinimide (288 mg, 1.01 mmol) was added and the mixture was warmed to rt and stirred for 16 h. The solvent was removed and the residual material was purified by column chromatography on silica gel, first eluting with EtOAc, followed by a gradient of MeOH in EtOAc (0.1% Et₃N) to afford Compound 539 (222 mg, 65%) as an oil. TLC: R_{f}=0.16 (EtOAc MeOH, 1:1 v/v); LC-MS (+ve mode): m/z=360.20 [M+H]⁺; ¹H NMR (300 MHz, CDCl₃) δ 8.37 (d, J=8.4 Hz, 1H, ArH), 7.47 (m, 1H, ArH), 7.28 (m, 3H, 3×ArH), 5.42 (d, J=6.8 Hz, 1H, NH), 5.00 (m, 1H, CH), 2.80 (m, 2H, CH₂), 2.59 (m, 2H, CH₂), 2.28 (s, 6H, 2×NMe) 1.38 (s, 12H, ^{t}Bu, CH₃).

### Example 1-32: (S)-2-amino-1-(3-(2-(dimethylamino)ethyl)-1H-indol-1-yl)propan-1-one dihydrochloride (Compound 540)

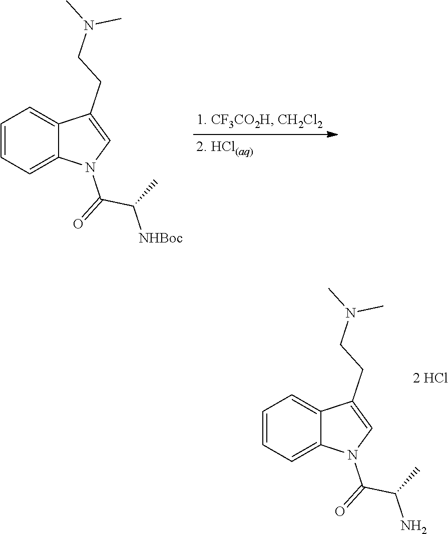 [SMILES: C[C@H](NC(=O)OC(C)(C)C)C(=O)n1cc(CCN(C)C)c2ccccc21 ; C[C@H](N)C(=O)n1cc(CCN(C)C)c2ccccc21]

TFA (4.07 g, 2.72 mL, 35.6 mmol) was added to a solution of (S)-tert-butyl (1-(3-(2-(dimethylamino)ethyl)-1H-indol-1-yl)-1-oxopropan-2-yl)carbamate (Compound 539, 256 mg, 0.71 mmol) in DCM (5 mL) at rt and stirring was continued for 4 h. The mixture was concentrated and azeotroped with CHCl₃ (4×10 mL) and MeOH (10 mL). The residue was dissolved in 1M HCl (2 mL, 2 mmol) and purified by reversed-phase chromatography on silica eluting with a gradient of acetonitrile in 0.02% HCl_{(aq.)} to afforded Compound 540 (236 mg, quant.) as a solid. LC-MS (+ve mode): m/z=260.15 [M+H]⁺; ¹H NMR (300 MHz, CD₃OD) δ 8.43 (m, 1H, ArH), 7.81 (s, 1H, ArH), 7.71 (m, 1H, ArH), 7.41 (m, 2H, ArH), 4.98 (q, J=7.1 Hz, 1H, CH), 3.55 (m, 2H, CH₂), 3.24 (m, 2H, CH₂), 3.00 (s, 6H, Hz, 2×NMe), 1.70 (d, J=7.1 Hz, 3H, CH₃); ¹³C NMR (75.5 MHz, CD₃OD) δ 168.3, 136.0, 129.8, 125.8, 124.5, 122.2, 118.8, 118.7, 113.6, 56.3, 49.0, 42.2, 20.0, 16.2, 7.9.

### Example 1-33: (S)-tert-butyl (1-(3-(2-(dimethylamino)ethyl)-5-methoxy-1H-indol-1-yl)-1-oxopropan-2-yl)carbamate (Compound 541)

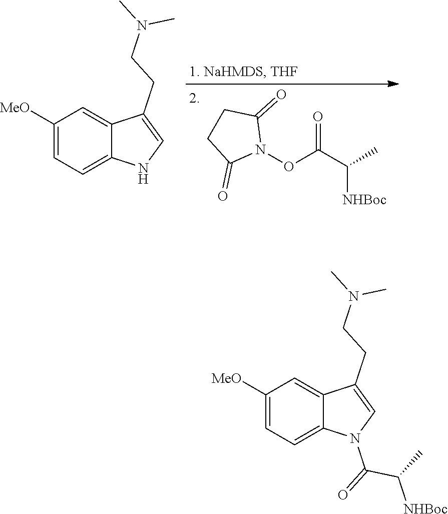

NaHMDS, 1M in THF (0.74 mL, 0.74 mmol) was added to a stirred mixture of 5-OMe-DMT (154 mg, 71.0 mmol) in anhydrous THF (5 mL) at −78° C. After 30 min, Boc-alanine-O-succinimide (193 mg, 0.67 mmol) was added and the mixture was warmed to rt and stirred for 16 h. The solvent was removed, and the residue was purified by column chromatography on silica gel, first eluting with EtOAc, followed by a gradient of MeOH in EtOAc (0.1% Et₃N) to afford Compound 541 (132 mg, 47%) as an oil. TLC: R_{f}=0.18 (EtOAc-MeOH, 1:1 v/v); LC-MS (+ve mode): m/z=390.20 [M+H]⁺; ¹H NMR (300 MHz, CDCl₃) δ 8.25 (d, J=8.9 Hz, 1H, ArH), 7.27 (s, 1H, ArH), 6.90 (m, 2H, 2×ArH), 5.38 (d, J=8.8 Hz, 1H, NH), 5.00 (m, 1H, CH), 3.81 (s, 3H, OMe), 2.82 (m, 2H, CH₂), 2.62 (m, 2H, CH₂), 2.32 (s, 6H, 2×NMe) 1.39 (s, 12H, ^{t}Bu, CH₃).

### Example 1-34: (S)-2-amino-1-(3-(2-(dimethylamino)ethyl)-5-methoxy-1H-indol-1-yl)propan-1-one dihydrochloride (Compound 542)

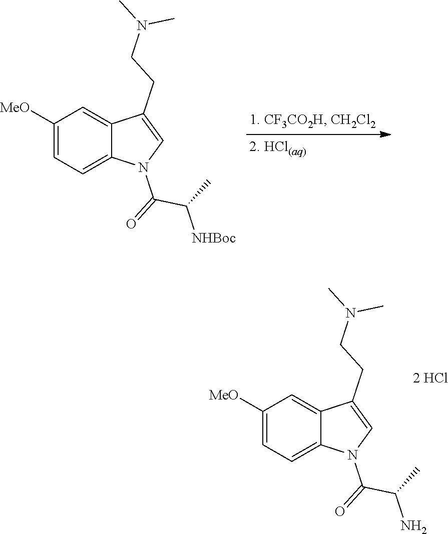 [SMILES: COc1ccc2c(c1)c(CCN(C)C)cn2C(=O)[C@H](C)NC(=O)OC(C)(C)C ; COc1ccc2c(c1)c(CCN(C)C)cn2C(=O)[C@H](C)N]

TFA (1.88 g, 1.26 mL, 16.5 mmol) was added to a solution of (S)-tert-butyl (1-(3-(2-(dimethylamino)ethyl)-5-methoxy-1H-indol-1-yl)-1-oxopropan-2-yl)carbamate (Compound 541, 132 mg, 0.33 mmol) in DCM (5 mL) at rt and stirring was continued for 4 h. The mixture was concentrated and azeotroped with CHCl₃ (4×10 mL) and MeOH (10 mL). The residue was dissolved in 1M HCl (2 mL, 2 mmol) and purified by reversed-phase chromatography on silica eluting with a gradient of acetonitrile in 0.02% HCl_{(aq.)} to give the product (40 mg, 72%) as a solid. LC-MS (+ve mode): m/z=290.15 [M+H]⁺; ¹H NMR (300 MHz, CD₃OD) δ 8.22 (d, J=9.0 Hz, 1H, ArH), 7.76 (s, 1H, ArH), 7.13 (d, J=2.4 Hz, 1H, ArH), 6.92 (dd, J=9.0, 2.4 Hz, 1H, ArH), 4.92 (m, 1H, CH), 3.80 (s, 3H, OMe), 3.47 (m, 2H, CH₂), 3.13 (m, 2H, CH₂), 2.91 (d, J=3.0 Hz, 6H, 2×NMe), 1.61 (d, J=7.2 Hz, 3H, CH₃); ¹³C NMR (75.5 MHz, CD₃OD) δ 157.6, 131.0, 130.4, 123.0, 118.8, 117.1, 113.8, 102.8, 101.9, 56.3, 54.9, 48.9, 42.2, 42.2, 20.1, 16.4.

### Example 1-35: (S)-tert-butyl (1-(3-(2-(dimethylamino)ethyl)-1H-indol-1-yl)-1-oxo-3-phenylpropan-2-yl)carbamate (Compound 543)

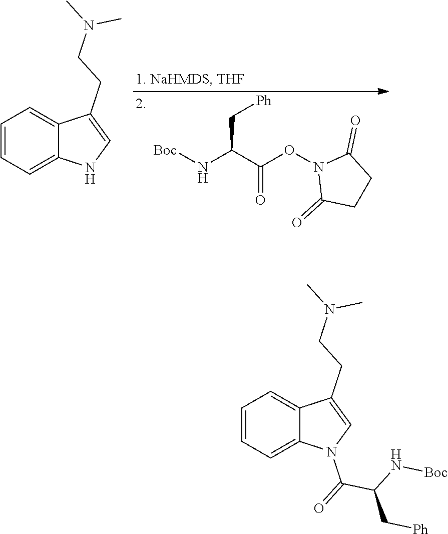 [SMILES: CN(C)CCc1c[nH]c2ccccc12 ; CC(C)(C)OC(=O)N[C@@H](Cc1ccccc1)C(=O)ON1C(=O)CCC1=O ; CN(C)CCc1cn(C(=O)[C@H](Cc2ccccc2)NC(=O)OC(C)(C)C)c2ccccc12]

NaHMDS, 1M in THF (0.97 mL, 0.97 mmol) was added to a stirred mixture of DMT (173 mg, 0.92 mmol) in anhydrous THF (13 mL) at −78° C. After 30 min, the resulting mixture was added dropwise to Boc-Phenylalanine-OSu (300 mg, 0.83 mmol) and stirring was continued at rt for 16 h. The mixture was concentrated to dryness before being dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated. The residue was purified by column chromatography on silica gel eluting with a gradient of MeOH/DCM to Compound 543 (111 mg, 31%) as a semi-solid. LC-MS (+ve mode): m/z=436.20 [M+H]⁺.

### Example 1-36: (S)-tert-butyl (1-(3-(2-(dimethylamino)ethyl)-1H-indol-1-yl)-3-methyl-1-oxobutan-2-yl)carbamate (Compound 544)

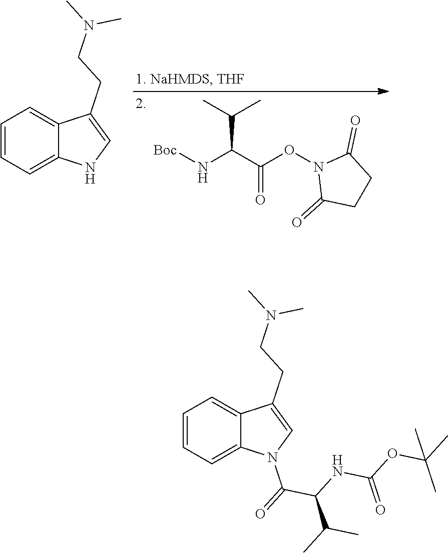 [SMILES: CN(C)CCc1c[nH]c2ccccc12 ; CC(C)[C@H](NC(=O)OC(C)(C)C)C(=O)ON1C(=O)CCC1=O ; CC(C)[C@H](NC(=O)OC(C)(C)C)C(=O)n1cc(CCN(C)C)c2ccccc21]

NaHMDS, 1M in THF (0.97 mL, 0.97 mmol) was added to a stirred solution of DMT (173 mg, 0.92 mmol) in anhydrous THF (13 mL) at −78° C. After 30 min the mixture was added dropwise to Boc-Valine-OSu (260 mg, 0.83 mmol) and the mixture was warmed to rt and stirred for 16 h. The mixture was concentrated to dryness before being dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated. The residue was purified by column chromatography on silica gel, eluting with a gradient of MeOH in DCM to give the product (245 mg, 76%) as a semi-solid. LC-MS (+ve mode): m/z=388.20 [M+H]⁺; ¹H NMR (300 MHz, CDCl₃) δ 8.39 (d, J=9.0 Hz, 1H, ArH), 7.53 (d, J=9.0 Hz, 1H, ArH), 7.44 (s, 1H, ArH), 7.30 (m, 2H, ArH), 5.27 (d, J=12.0 Hz, 1H, NH), 4.82 (m, 1H, CH), 3.33 (m, 4H, 2×CH₂), 2.87 (d, J=6.0 Hz, 6H, 2×NMe), 2.12 (m, 1H, CH), 1.38 (s, 9H, ^{t}Bu), 0.98 (d, J=6.0 Hz, 3H, CH₃), 0.89 (d, J=6.0 Hz, 3H, CH₃).

### Example 1-37: (S)-2-amino-1-(3-(2-(dimethylamino)ethyl)-1H-indol-1-yl)-3-methylbutan-1-one dihydrochloride (Compound 545)

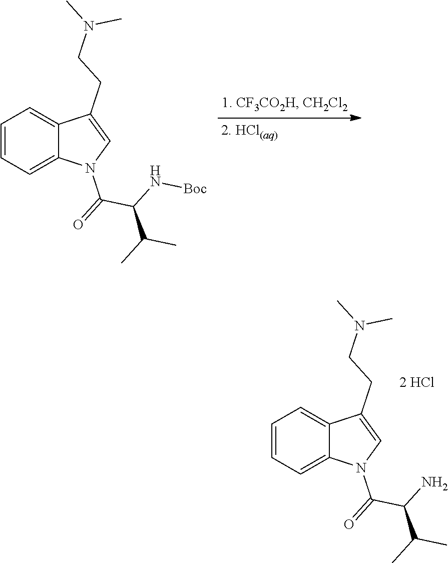 [SMILES: CC(C)[C@H](NC(=O)OC(C)(C)C)C(=O)n1cc(CCN(C)C)c2ccccc21 ; CC(C)[C@H](N)C(=O)n1cc(CCN(C)C)c2ccccc21]

(S)-tert-butyl (1-(3-(2-(dimethylamino)ethyl)-1H-indol-1-yl)-3-methyl-1-oxobutan-2-yl)carbamate (Compound 544, 245 mg, 0.63 mmol) was dissolved into DCM (12.5 mL) at rt and TFA (2.42 mL, 31.6 mmol) was added. The mixture was stirred at rt for 3 h, then concentrated under reduced pressure, azeotroping with CHCl₃ (4×10 mL). The residue was purified by column chromatography on silica gel, eluting with a gradient of MeOH in CH₂Cl₂. This material was further purified by reversed-phase chromatography on silica eluting with a gradient of acetonitrile in 0.02% HCl_{(aq.)} to afforded Compound 545 (85.3 mg, 38%) as a solid. LC-MS (+ve mode): m/z=288.15 [M+H]⁺; ¹H NMR (300 MHz, D₂O) δ 8.31 (d, J=6.0 Hz, 1H, ArH), 7.62 (m, 2H, 2×ArH), 7.41 (m, 2H, 2×ArH), 4.82 (d, J=6.0 Hz, 1H, CH), 3.46 (m, 2H, CH₂), 3.17 (m, 2H, CH₂), 2.89 (d, J=1.7 Hz, 6H, 2×NMe), 2.43 (m, 1H, CH), 1.07 (d, J=9.0 Hz, 3H, CH₃), 0.94 (d, J=9.0 Hz, 3H, CH₃); ¹³C NMR (75.5 MHz, CDCl₃) δ 168.2, 135.6, 129.8, 126.4, 125.1, 122.8, 119.2, 119.0, 116.4, 57.9, 56.3, 42.8, 42.7, 30.2, 20.0, 18.0, 16.0.

### Example 1-38: (S)-tert-butyl (1-(3-(2-(dimethylamino)ethyl)-5-methoxy-1H-indol-1-yl)-3-methyl-1-oxobutan-2-yl)carbamate (Compound 546)

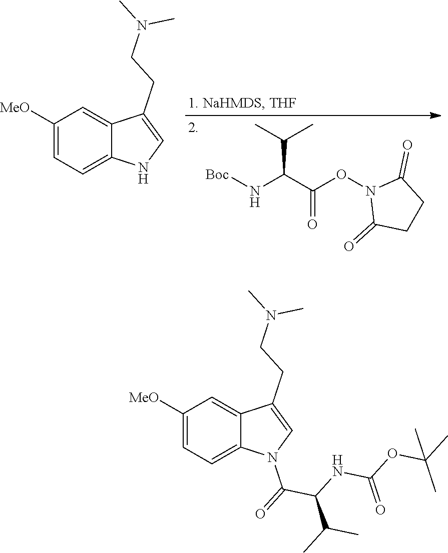 [SMILES: COc1ccc2[nH]cc(CCN(C)C)c2c1 ; CC(C)[C@H](NC(=O)OC(C)(C)C)C(=O)ON1C(=O)CCC1=O ; COc1ccc2c(c1)c(CCN(C)C)cn2C(=O)[C@@H](NC(=O)OC(C)(C)C)C(C)C]

NaHMDS, 1M in THF (0.97 mL, 0.97 mmol) was added to a stirred solution of 5-OMe-DMT (210 mg, 0.92 mmol) in anhydrous THF (13 mL) at −78° C. After 30 min the mixture was added dropwise to Boc-valine-OSu (260 mg, 0.83 mmol) and the mixture was warmed to rt and stirred for 16 h. The mixture was concentrated to dryness, then dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated to give an oil. The residue was purified by column chromatography on silica gel, eluting with a gradient of MeOH in DCM to afford Compound 546 (216 mg, 56%) as a semi-solid. LC-MS (+ve mode): m/z=418.25 [M+H]⁺; ¹H NMR (300 MHz, CDCl₃) δ 8.27 (d, J=8.9 Hz, 1H, ArH), 7.30 (s, 1H, ArH), 6.95 (d, J=2.4 Hz, 1H, ArH), 6.89 (dd, J=8.9, 2.4 Hz, 1H, ArH), 5.30 (d, J=9.2 Hz, 1H, CH), 4.83 (m, 1H, CH), 3.81 (s, 3H, OMe), 2.82 (m, 2H, CH₂), 2.62 (m, 2H, CH₂), 2.33 (s, 6H, 2×NCH₃), 2.13 (m, 1H, CH), 1.38 (s, 9H, 3×CH₃), 0.97 (d, J=6.8 Hz, 3H, CH₃), 0.87 (d, J=6.8 Hz, 3H, CH₃).

### Example 1-39: (S)-2-amino-1-(3-(2-(dimethylamino)ethyl)-5-methoxy-1H-indol-1-yl)-3-methylbutan-1-one dihydrochloride (Compound 547)

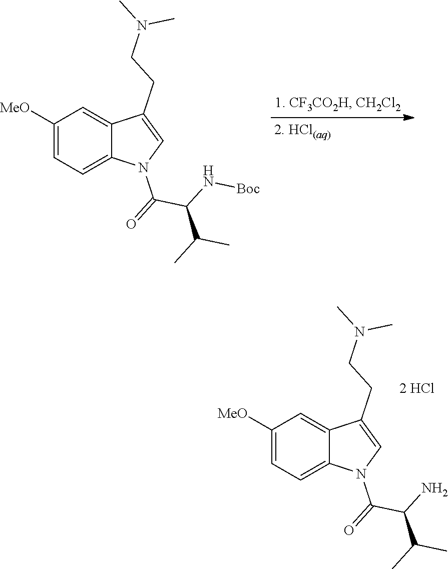 [SMILES: COc1ccc2c(c1)c(CCN(C)C)cn2C(=O)[C@@H](NC(=O)OC(C)(C)C)C(C)C ; COc1ccc2c(c1)c(CCN(C)C)cn2C(=O)[C@@H](N)C(C)C]

(S)-tert-butyl (1-(3-(2-(dimethylamino)ethyl)-5-methoxy-1H-indol-1-yl)-3-methyl-1-oxobutan-2-yl)carbamate (Compound 546, 216 mg, 0.52 mmol) was dissolved into DCM (11 mL) at rt and TFA (2.95 g, 1.98 mL, 25.9 mmol) was added. The mixture was stirred at rt for 1 h, then the solvent was removed under reduced pressure, azeotroping with CHCl₃ (4×10 mL). The crude residue was purified by reversed-phase chromatography on silica eluting with a gradient of acetonitrile in 0.02% HCl_{(aq.)} to afford Compound 547 (168 mg, 83%) as an oil. LC-MS (+ve mode): m/z=318.15 [M+H]⁺; ¹H NMR (300 MHz, D₂O) δ 8.20 (d, J=9.0 Hz, 1H, ArH), 7.58 (s, 1H, ArH), 7.11 (d, J=2.4 Hz, 1H, ArH), 7.01 (dd, J=9.0, 2.4 Hz, 1H, ArH), 4.78 (d, J=5.1 Hz, 1H, CH), 3.82 (s, 3H, OMe), 3.45 (m, 2H, CH₂), 3.12 (m, 2H, CH₂), 2.89 (d, J=1.8 Hz, 6H, 2×NCH₃), 2.41 (m, 1H, CH), 1.05 (d, J=6.9 Hz, 3H, CH₃), 0.94 (d, J=6.9 Hz, 3H, CH₃); ¹³C NMR (75.5 MHz, CDCl₃) δ 167.7, 156.6, 131.1, 130.3, 123.6, 118.9, 117.4, 113.9, 102.8, 57.7, 56.2, 55.9, 42.7, 30.3, 20.0, 18.0, 16.0.

### Example 1-40: (S)-2-amino-1-(3-(2-(dimethylamino)ethyl)-1H-indol-1-yl)-3-phenylpropan-1-one bis-hydrochloride (Compound 548)

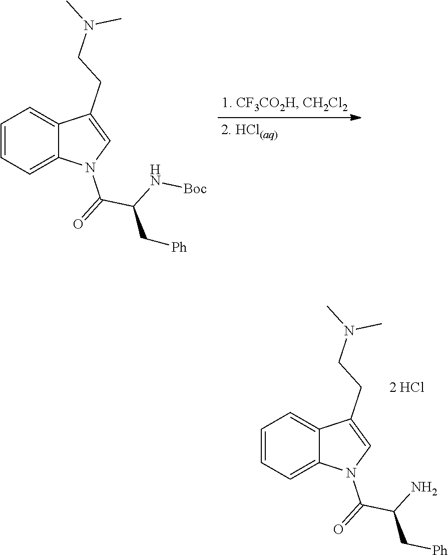 [SMILES: CN(C)CCc1cn(C(=O)[C@H](Cc2ccccc2)NC(=O)OC(C)(C)C)c2ccccc12 ; CN(C)CCc1cn(C(=O)[C@@H](N)Cc2ccccc2)c2ccccc12]

(S)-Tert-butyl (1-(3-(2-(dimethylamino)ethyl)-1H-indol-1-yl)-1-oxo-3-phenylpropan-2-yl)carbamate (Compound 543, 111 mg, 0.25 mmol) was dissolved into DCM (5 mL) and TFA (1.43 g, 0.96 mL, 12.5 mmol) was added. The reaction mixture was stirred at rt for 1 h, then the solvent was removed under reduced pressure, azeotroping the residue with CHCl₃ (4×10 mL). The residue was purified by reversed-phase chromatography on silica eluting with a gradient of acetonitrile in 0.02% hydrochloric acid to afford Compound 548 as a bis-hydrochloride salt (40.8 mg, 48%) as a solid. LC-MS (+ve mode): m/z=336.15 [M+H]⁺; ¹H NMR (300 MHz, D₂O) δ 8.29 (d, J=6.0 Hz, 1H, ArH), 7.55 (d, J=9.0 Hz, 1H, ArH), 7.40 (m, 2H, ArH), 7.10 (m, 6H, ArH), 5.12 (dd, J=9.0, 6.0 Hz, 1H, CH), 3.33 (dd, J=13.8, 5.7 Hz, 1H, 0.5×CH₂), 3.21 (m, 3H, 0.5×CH₂+CH₂), 2.91 (m, 8H, 2×NCH₃ and CH₂); ¹³C NMR (75.5 MHz, D₂O) δ 168.0, 135.2, 133.5, 129.7, 129.3, 129.1, 128.9, 128.0, 126.3, 125.1, 122.3, 119.1, 118.6, 116.4, 56.3, 53.9, 42.8, 42.6, 37.5, 19.7.

### Example 1-41: (S)-tert-butyl (1-(3-(2-(dimethylamino)ethyl)-5-methoxy-1H-indol-1-yl)-1-oxo-3-phenylpropan-2-yl)carbamate (Compound 549)

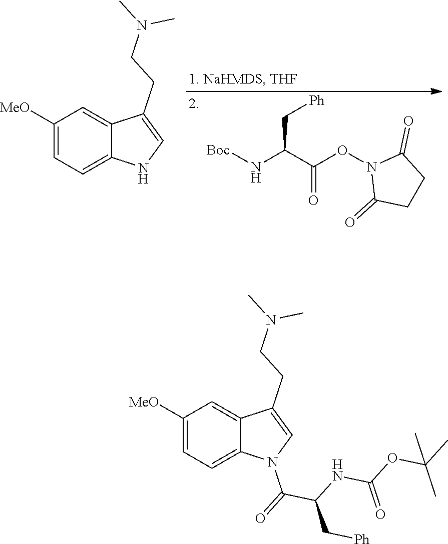 [SMILES: COc1ccc2[nH]cc(CCN(C)C)c2c1 ; CC(C)(C)OC(=O)N[C@@H](Cc1ccccc1)C(=O)ON1C(=O)CCC1=O ; COc1ccc2c(c1)c(CCN(C)C)cn2C(=O)[C@H](Cc1ccccc1)NC(=O)OC(C)(C)C]

NaHMDS, 1M in THF (0.97 mL, 0.97 mmol) was added to a stirred solution of 5-OMe-DMT (210 mg, 0.96 mmol) in anhydrous THF (13 mL) at −78° C. After 30 min, the resulting mixture was added dropwise to Boc-phenylalanine-OSu (300 mg, 0.83 mmol) and stirring was continued at rt for 16 h. The reaction mixture was concentrated to dryness before being dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated to give a crude oil. The residue was purified by column chromatography on silica gel, eluting with a gradient of MeOH in DCM to afford Compound 549 (261 mg, 67%) as a semi-solid. LC-MS (+ve mode): m/z=466.25 [M+H]⁺.

### Example 1-42: (S)-2-amino-1-(3-(2-(dimethylamino)ethyl)-5-methoxy-1H-indol-1-yl)-3-phenylpropan-1-one bis-hydrochloride (Compound 550)

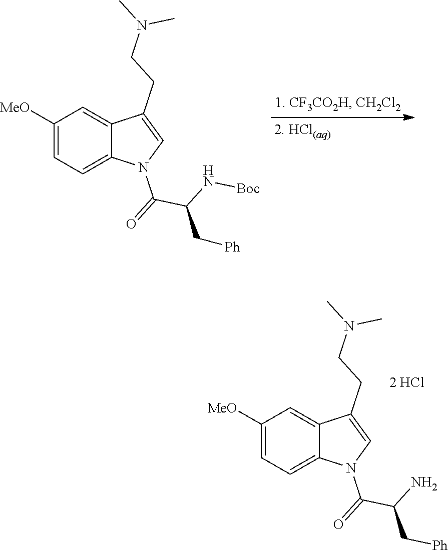 [SMILES: COc1ccc2c(c1)c(CCN(C)C)cn2C(=O)[C@H](Cc1ccccc1)NC(=O)OC(C)(C)C ; COc1ccc2c(c1)c(CCN(C)C)cn2C(=O)[C@@H](N)Cc1ccccc1]

(S)-tert-butyl (1-(3-(2-(dimethylamino)ethyl)-5-methoxy-1H-indol-1-yl)-1-oxo-3-phenylpropan-2-yl)carbamate (Compound 549, 261 mg, 0.56 mmol) was dissolved into DCM (11 mL) and TFA (3.19 g, 2.14 mL, 28.0 mmol) was added. The reaction mixture was stirred at rt for 1 h, then the solvent was removed under reduced pressure, azeotroping with CHCl₃ (4×10 mL). The residue was purified by reversed-phase chromatography on silica eluting with a gradient of acetonitrile in 0.02% hydrochloric acid to afford Compound 550 as a bis-hydrochloride salt (244 mg, 83%) as a solid. LC-MS (+ve mode): m/z=366.20 [M+H]⁺; ¹H NMR (300 MHz, D₂O) δ 8.17 (d, J=9.9 Hz, 1H, ArH), 7.13 (m, 3H, 3×ArH), 7.01 (m, 5H, 5×ArH), 5.07 (dd, J=9.3, 5.7 Hz, 1H, CH), 3.81 (s, 3H, OMe), 3.38 (dd, J=13.5, 5.7 Hz, 1H, 0.5×CH₂), 3.18 (m, 3H, 0.5×CH₂+CH₂), 2.89 (m, 8H, 2×NCH₃ and CH₂); ¹³C NMR (75.5 MHz, D₂O) δ 167.5, 164.9, 156.6, 133.1, 131.1, 129.9, 129.3, 128.9, 128.0, 118.5, 117.4, 113.7, 102.7, 56.2, 55.8, 53.7, 42.8, 42.6, 37.6, 36.9, 31.3, 19.6.

### Example 1-43: 2-(Dimethylamino)-1-(3-(2-(dimethylamino)ethyl)-1H-indol-1-yl)ethan-1-one hydrochloride (Compound 551)

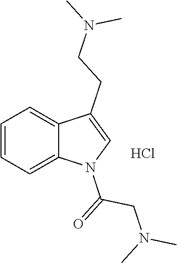 [SMILES: CN(C)CCc1cn(C(=O)CN(C)C)c2ccccc12 ; Cl]

To a solution of N,N-dimethyltryptamine (282 mg, 1.50 mmol) in anhydrous THF (20 mL) at −78° C. under an atmosphere of N2 was added NaHMDS, 1M in THF (6.0 mL, 6.0 mmol) and the mixture was stirred at −78° C. for 30 min. 2-(Dimethylamino)acetyl chloride hydrochloride (475 mg, 3.00 mmol) was added and the mixture was stirred at −78° C. for 5 min, then warmed to rt and stirred for 4 h. H₂O (3 mL) was added and the mixture was concentrated, and the residue was purified by reversed-phase chromatography, eluting with 0 to 100% acetonitrile in 0.02% hydrochloric acid to give 2-(dimethylamino)-1-(3-(2-(dimethylamino)ethyl)-1H-indol-1-yl)ethan-1-one HCl (Compound 551, 79 mg, 17%) as a solid. LC-MS (+ve mode): m/z=274.15 [M+H]⁺; ¹H NMR (300 MHz, D₂O) δ 8.37 (br, 1H, ArH), 7.73 (br, 1H, ArH), 7.51 (m, 3H, 3×ArH), 4.89 (s, 2H, CH₂), 3.56 (m, 2H, CH₂), 3.26 (m, 2H, CH₂), 3.14 (s, 6H, 2×NMe), 2.98 (s, 6H, 2×NMe); ¹³C NMR (75.5 MHz, D₂O) δ 163.6, 134.9, 129.6, 126.3, 124.9, 121.9, 121.9, 119.3, 119.1, 59.0, 56.4, 44.2, 42.8, 20.0.

### Example 1-44: 2-(Dimethylamino)-1-(3-(2-(dimethylamino)ethyl)-5-methoxy-1H-indol-1-yl)ethan-1-one formate (Compound 552)

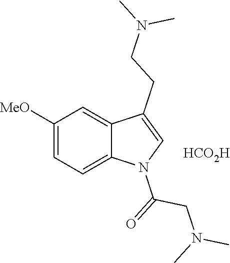 [SMILES: COc1ccc2c(c1)c(CCN(C)C)cn2C(=O)CN(C)C ; "HCO2H"]

To a solution of 5-methoxy-N,N-dimethyltryptamine (229 mg, 1.05 mmol) in anhydrous THF (12 mL) at −78° C. under an atmosphere of N2 was added NaHMDS, 1M in THF (5.5 mL, 5.5 mmol) and the mixture was stirred at −78° C. for 30 min. 2-(Dimethylamino)acetyl chloride hydrochloride (0.67 g, 4.2 mmol) was added and the mixture was stirred at −78° C. for 10 min, then warmed to rt and stirred for 3 h. H₂O (2 mL) was added, the mixture was concentrated and the residue was purified by reversed-phase chromatography, eluting with 0 to 100% acetonitrile in 0.1% formic acid to give 2-(dimethylamino)-1-(3-(2-(dimethylamino)ethyl)-5-methoxy-1H-indol-1-yl)ethan-1-one formate (Compound 552, 108 mg, 29%) as a solid. LC-MS (+ve mode): m/z=304.15 [M+H]⁺; ¹H NMR (300 MHz, D₂O) δ 8.27 (br, 1H, ArH), 7.50 (s, 1H, ArH), 7.21 (d, J=2.5 Hz, 1H, ArH), 7.12 (dd, J=9.0, 2.5 Hz, 1H, ArH), 4.87 (s, 2H, CH₂), 3.93 (s, 3H, OMe), 3.55 (m, 2H, CH₂), 3.22 (m, 2H, CH₂), 3.14 (s, 6H, 2×NMe), 2.98 (s, 6H, 2×NMe); ¹³C NMR (75.5 MHz, D₂O) δ 163.1, 156.5, 130.2, 122.7, 119.0, 114.0, 110.7, 110.0, 102.8, 56.2, 55.9, 44.2, 43.5, 42.8, 20.0.

### Example 1-45: (S)-2-amino-N-(2-(3-(2-(dimethylamino)ethyl)-1H-indol-1-yl)-2-oxoethyl)-N-methyl-3-phenylpropanamide (Compound 553)

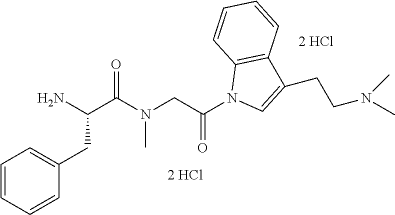 [SMILES: CN(C)CCc1cn(C(=O)CN(C)C(=O)[C@@H](N)Cc2ccccc2)c2ccccc12]

### Step 1: (S)-2,5-dioxopyrrolidin-1-yl 2-(2-((tert-butoxycarbonyl)amino)-N-methyl-3-phenylpropanamido)acetate

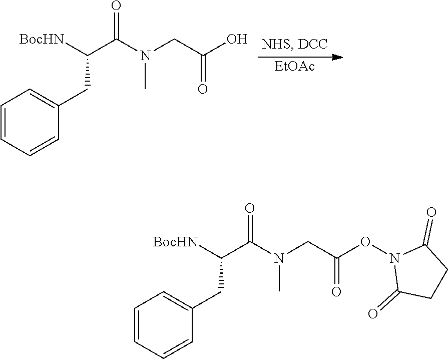 [SMILES: "EtOAc" ; CN(CC(=O)O)C(=O)[C@H](Cc1ccccc1)NC(=O)OC(C)(C)C ; CN(CC(=O)ON1C(=O)CCC1=O)C(=O)[C@H](Cc1ccccc1)NC(=O)OC(C)(C)C]

Boc-phenylalanine-N-methyl-glycine [CAS No: 108787-68-4] (500 mg, 1.49 mmol) and N-hydroxysuccinimide (188.5 mg, 1.63 mmol) were dissolved in EtOAc (50 mL) and cooled to 0° C. Dicyclohexylcarbodiimide (338 mg, 1.64 mmol) was added and the mixture was stirred at 0° C. for 2 h, then allowed to rt and stirred overnight. The mixture was filtered through Celite and the filtrate was concentrated to give the product (766 mg, quant) as a solid, which was used without further purification. LC-MS (+ve mode): m/z=434.15 [M+H]⁺.

### Step 2: (S)-tert-butyl (1-((2-(3-(2-(dimethylamino)ethyl)-1H-indol-1-yl)-2-oxoethyl)(methyl)amino)-1-oxo-3-phenylpropan-2-yl)carbamate

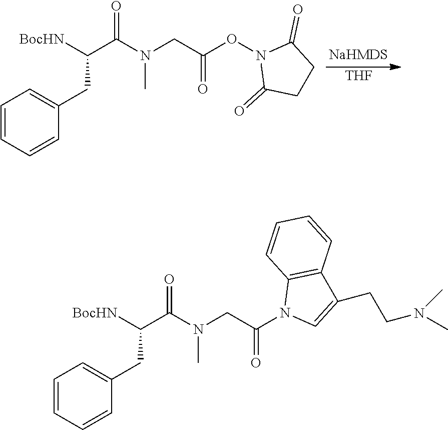 [SMILES: CN(CC(=O)ON1C(=O)CCC1=O)C(=O)[C@H](Cc1ccccc1)NC(=O)OC(C)(C)C ; CN(C)CCc1cn(C(=O)CN(C)C(=O)[C@H](Cc2ccccc2)NC(=O)OC(C)(C)C)c2ccccc12 ; "THF"]

NaHMDS, 1M in THF (1.67 mL, 1.67 mmol) was added to a stirred solution of DMT (200 mg, 1.06 mmol) in anhydrous THF (5 mL) at −78° C. After 30 min, a solution of Boc-phenylalanine-N-methyl-glycine-OSu (367 mg, 0.85 mmol) in THF (5 mL) was added and the mixture was warmed to rt and stirred for 16 h. The solvent was removed and the residual material was purified by column chromatography on silica gel, eluting with a gradient of MeOH in DCM to the product (33 mg, 8%) as a solid. LC-MS (+ve mode): m/z=507.30 [M+H]⁺; ¹H NMR (300 MHz, CDCl₃) δ 8.32 (d, J=7.8 Hz, 1H, ArH), 7.51 (m, 1H, ArH), 7.32 (m, 6H, 6×ArH), 7.00 (m, 2H, 2×ArH), 5.56 (m, 1H, NH), 4.90 (m, 1H, CH), 4.61 (s, 2H, CH₂), 3.07 (m, 6H, 3×CH₂), 2.95 (s, 3H, NMe), 2.59 (s, 6H, 2×NMe), 1.34, (s, 9H, ^{t}Bu).

### Step 3: (S)-2-amino-N-(2-(3-(2-(dimethylamino)ethyl)-1H-indol-1-yl)-2-oxoethyl)-N-methyl-3-phenylpropanamide dihydrochloride

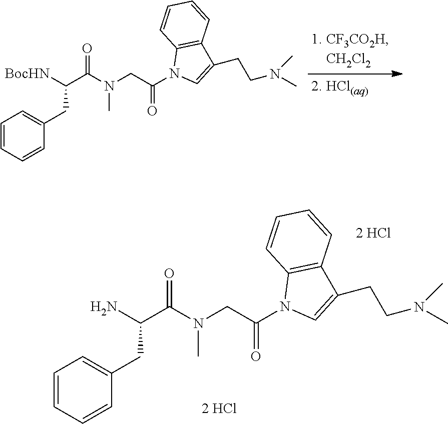 [SMILES: CN(C)CCc1cn(C(=O)CN(C)C(=O)[C@H](Cc2ccccc2)NC(=O)OC(C)(C)C)c2ccccc12 ; CN(C)CCc1cn(C(=O)CN(C)C(=O)[C@@H](N)Cc2ccccc2)c2ccccc12]

TFA (0.57 g, 0.38 mL, 5.03 mmol) was added to a mixture of (S)-tert-butyl (1-((2-(3-(2-(dimethylamino)ethyl)-1H-indol-1-yl)-2-oxoethyl)(methyl)amino)-1-oxo-3-phenylpropan-2-yl)carbamate (51 mg, 0.10 mmol) in DCM (1.4 mL) at rt and stirring was continued for 3 h. The mixture was concentrated and azeotroped with CHCl₃ (4×10 mL) and the residue was purified by reversed-phase chromatography on silica eluting with a gradient of acetonitrile in 0.02% HCl_{(aq.)} to give the product as a bis-hydrochloride salt (40 mg, 83%) an oil. LC-MS (+ve mode): m/z=407.25 [M+H]⁺; ¹H NMR (300 MHz, CD₃OD) δ 8.42 (m, 1H, ArH), 7.80 (m, 1H, ArH), 7.73 (m, 1H, ArH), 7.41 (m, 7H, 7×ArH), 4.97 (obs, 2H, CH₂), 4.83 (m, 1H, CH), 3.57 (m, 2H, CH₂), 3.07 (m, 4H, 2×CH₂), 3.08 (s, 3H, NMe), 3.02 (s, 6H, 2×NMe).

### Example 1-46: (S)-2-amino-N-(2-(3-(2-(dimethylamino)ethyl)-5-methoxy-1H-indol-1-yl)-2-oxoethyl)-N-methyl-3-phenylpropanamide (Compound 554)

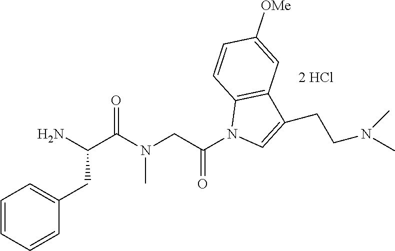 [SMILES: COc1ccc2c(c1)c(CCN(C)C)cn2C(=O)CN(C)C(=O)[C@@H](N)Cc1ccccc1]

### Step 1: (S)-tert-butyl (1-((2-(3-(2-(dimethylamino)ethyl)-5-methoxy-1H-indol-1-yl)-2-oxoethyl)(methyl)amino)-1-oxo-3-phenylpropan-2-yl)carbamate

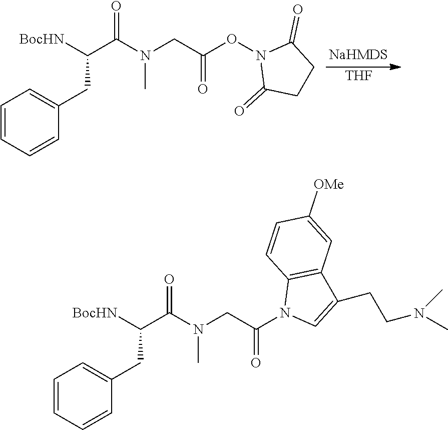 [SMILES: CN(CC(=O)ON1C(=O)CCC1=O)C(=O)[C@H](Cc1ccccc1)NC(=O)OC(C)(C)C ; COc1ccc2c(c1)c(CCN(C)C)cn2C(=O)CN(C)C(=O)[C@H](Cc1ccccc1)NC(=O)OC(C)(C)C ; "THF"]

NaHMDS, 1M in THF (0.96 mL, 0.96 mmol) was added to a stirred solution of 5-OMe-DMT (200 mg, 0.91 mmol) in anhydrous THF (5 mL) at −78° C. After 30 min, a solution of Boc-phenylalanine-N-methyl-glycine-OSu (317 mg, 0.73 mmol) in THF (5 mL) was added and the mixture was warmed to rt and stirred for 16 h. The solvent was removed and the residual material was purified by column chromatography on silica gel, eluting with a gradient of MeOH in DCM to give the product (55 mg, 11%) as a solid. LC-MS (+ve mode): m/z=537.25 [M+H]⁺; ¹H NMR (300 MHz, CDCl₃) δ 8.20 (d, J=9.0 Hz, 1H, ArH), 7.35 (s, 1H, ArH), 7.17 (m, 5H, 5×ArH), 7.24 (d, J=2.7 Hz, 1H, ArH), 7.90 (dd, J=9.0, 2.7 Hz, 2H, ArH), 5.27 (m, 1H, NH), 4.88 (m, 1H, CH), 4.60 (s, 2H, CH₂), 3.83 (s, 3H, OMe), 3.07 (m, 6H, 3×CH₂), 2.94 (s, 3H, NMe), 2.66 (s, 6H, 2×NMe), 1.34, (s, 9H, ^{t}Bu).

### Step 2: (S)-2-amino-N-(2-(3-(2-(dimethylamino)ethyl)-5-methoxy-1H-indol-1-yl)-2-oxoethyl)-N-methyl-3-phenylpropanamide

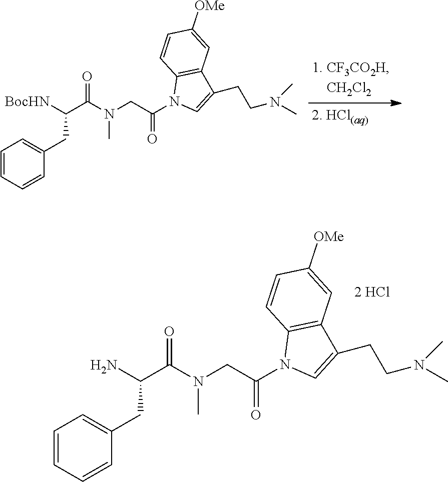 [SMILES: COc1ccc2c(c1)c(CCN(C)C)cn2C(=O)CN(C)C(=O)[C@H](Cc1ccccc1)NC(=O)OC(C)(C)C ; COc1ccc2c(c1)c(CCN(C)C)cn2C(=O)CN(C)C(=O)[C@@H](N)Cc1ccccc1]

TFA (0.58 g, 0.39 mL, 5.13 mmol) was added to a solution of (S)-tert-butyl (1-((2-(3-(2-(dimethylamino)ethyl)-5-methoxy-1H-indol-1-yl)-2-oxoethyl)(methyl)amino)-1-oxo-3-phenylpropan-2-yl)carbamate (55 mg, 0.10 mmol) in DCM (1.5 mL) at rt and stirring was continued for 2 h. The mixture was concentrated and azeotroped with CHCl₃ (4×10 mL) and the residue was purified by reversed-phase chromatography on silica eluting with a gradient of acetonitrile in 0.02% HCl_{(aq.)} to give the product (19 mg, 37%) an oil. LC-MS (+ve mode): m/z=437.30 [M+H]⁺.

### Example 1-47: 2,2-dimethyl-3-(pivaloyloxy)propyl 3-(2-(dimethylamino)ethyl)-5-methoxy-1H-indole-1-carboxylate formate (Compound 555)

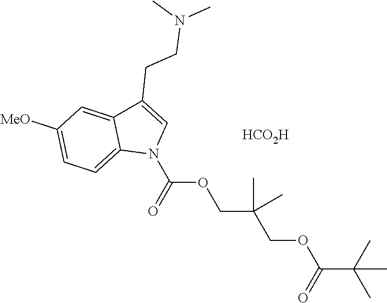 [SMILES: COc1ccc2c(c1)c(CCN(C)C)cn2C(=O)OCC(C)(C)COC(=O)C(C)(C)C ; "HCO2H"]

### Step 1: 3-((chlorocarbonyl)oxy)-2,2-dimethylpropyl pivalate

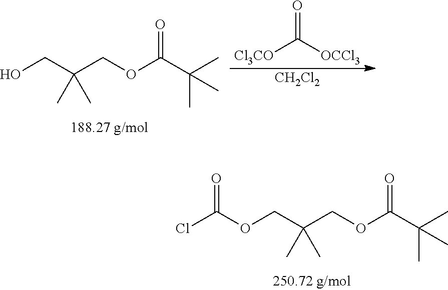

To a solution of 3-hydroxy-2,2-dimethylpropyl pivalate (346 mg, 1.84 mmol) in DCM (5 mL) was added DMAP (0.72 g, 5.81 mmol) and triphosgene (202 mg, 0.68 mmol) and the mixture was stirred at rt for 1 hour. This solution was used directly in the next step.

### Step 2: 2,2-dimethyl-3-(pivaloyloxy)propyl 3-(2-(dimethylamino)ethyl)-5-methoxy-1H-indole-1-carboxylate formate

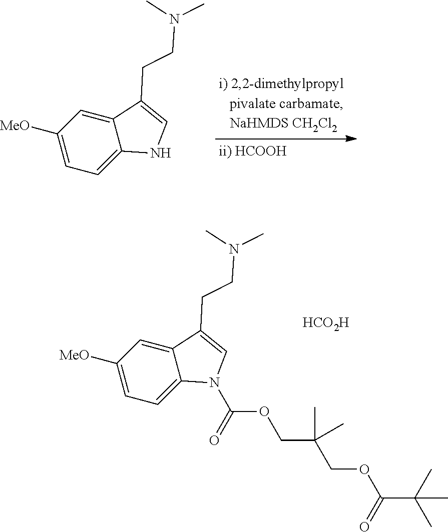 [SMILES: COc1ccc2[nH]cc(CCN(C)C)c2c1 ; COc1ccc2c(c1)c(CCN(C)C)cn2C(=O)OCC(C)(C)COC(=O)C(C)(C)C ; "HCO2H"]

NaHMDS, 1M in THF (0.96 mL, 0.96 mmol) was added to a stirred solution of 5-OMe-DMT (200 mg, 0.91 mmol) in DCM (10 mL) at −78° C. After 30 min, a solution of 3-((chlorocarbonyl)oxy)-2,2-dimethylpropyl pivalate (0.92 mmol) in DCM (5 mL) was added and the mixture stirred at −78° C. for 10 min, then the mixture warmed to rt and stirred for 16 h. The mixture was concentrated and the residual material was purified by column chromatography on silica gel, eluting with a gradient of MeOH in DCM followed by reversed-phase chromatography, eluting with 0 to 100% acetonitrile in 0.1% formic acid to afford Compound 555 (52 mg, 13%) as an oil. LC-MS (+ve mode): m/z=433.25 [M+H]⁺; ¹H NMR (300 MHz, CD₃OD) δ 8.42 (s, 1H, HCO₂H), 8.02 (d, J=9.0 Hz, 1H, ArH), 7.60 (s, 1H, ArH), 7.13 (d, J=2.5 Hz, 1H, ArH), 6.97 (dd, J=9.0, 2.5 Hz, 1H ArH), 4.27 (s, 2H, CH₂), 4.00 (s, 2H, CH₂), 3.86 (s, 3H, OMe), 3.44 (m, 2H, CH₂), 3.15 (m, 2H, CH₂), 2.93 (s, 6H, 2×CH₃), 1.19 (s, 9H, ^{t}Bu), 1.11 (s, 6H, 2×CH₃); ¹³C NMR (75.5 MHz, CDCl₃) δ 178.3, 167.4, 156.5, 150.4, 130.5, 123.7, 115.8, 115.5, 113.2, 101.5, 71.2, 68.6, 56.6, 54.8, 48.4, 48.2, 47.9, 47.6, 47.3, 47.0, 46.7, 42.2, 38.6, 35.0, 26.2, 20.7, 20.1.

### Example 1-48: 2,2-Dimethyl-3-(pivaloyloxy)propyl 3-(2-(dimethylamino)ethyl)-1H-indole-1-carboxylate formate (Compound 556)

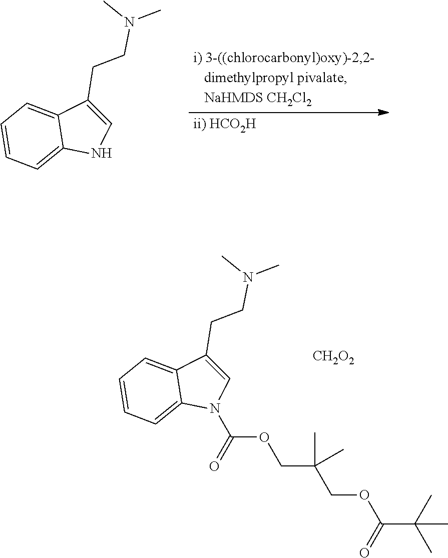 [SMILES: CN(C)CCc1c[nH]c2ccccc12 ; CN(C)CCc1cn(C(=O)OCC(C)(C)COC(=O)C(C)(C)C)c2ccccc12 ; "CH2O2"]

NaHMDS, 1M in THF (0.92 mL, 0.92 mmol) was added to a stirred solution of DMT (173 mg, 0.92 mmol) in DCM (10 mL) at −78° C. After 30 min, a solution of 3-((chlorocarbonyl)oxy)-2,2-dimethylpropyl pivalate (0.92 mmol) in DCM (5 mL) was added and the reaction mixture was stirred at −78° C. for 10 min, then warmed to rt and stirred for 16 h. The solvent was removed and the residual material was purified by column chromatography on silica gel, eluting with a gradient of MeOH in DCM, followed by reversed-phase chromatography, eluting with 0 to 100% acetonitrile in 0.1% formic acid to afford Compound 556 (97 mg, 23%) as an oil. LC-MS (+ve mode): m/z=403.25 [M+H]⁺; ¹H NMR (300 MHz, CD₃OD) δ 8.15 (d, J=7.9 Hz, 1H, ArH), 7.62 (m, 1H, ArH), 7.54 (s, 1H, ArH), 7.33 (m, 2H, 2×ArH), 4.31 (s, 2H, CH₂), 4.04 (s, 2H, CH₂), 3.00 (m, 2H, CH₂), 2.82 (m, 2H, CH₂), 2.46 (s, 6H, 2×NMe). 1.12 (s, 9H, ^{t}Bu), 1.14 (s, 6H, 2×CH₃); ¹³C NMR (75 MHz, CD₃OD) δ 203.4, 201.2, 178.4, 124.4, 122.6, 122.2, 122.1, 118.9, 118.6, 114.7, 69.0, 68.9, 58.4, 43.7, 38.6, 34.7, 26.2, 20.6.

### Example 1-49: 2-(1-di(dimethylamino)phosphoryl-indol-3-yl)-N,N-dimethyl-ethanamine (Compound 557)

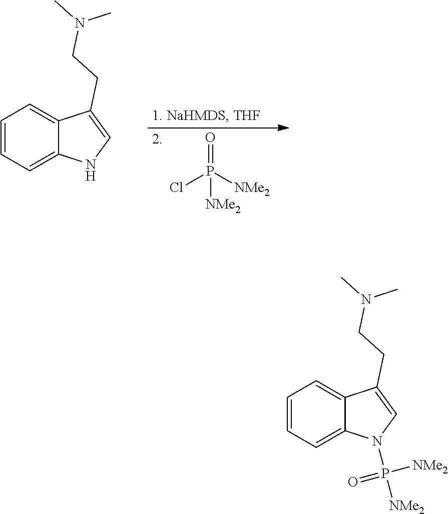 [SMILES: CN(C)CCc1c[nH]c2ccccc12 ; CN(C)P(=O)(Cl)N(C)C ; CN(C)CCc1cn(P(=O)(N(C)C)N(C)C)c2ccccc12]

NaHMDS, 1M in THF (1.12 mL, 1.12 mmol) was added to a stirred solution of DMT (200 mg, 1.06 mmol) in anhydrous THF (5 mL) at −78° C. After 30 min, N,N,N,N-tetramethylphosphorodiaminic chloride (181 mg, 0.16 mL, 1.06 mmol) was added and the mixture was warmed to rt and stirred for 16 h. The solvent was removed under vacuum and the crude residue was purified by column chromatography on silica gel, eluting with a gradient of MeOH in DCM to afford Compound 557 (251 mg, 74%) as an oil. TLC: R_{f}=0.55 (DCM-MeOH, 8:2 v/v); LC-MS (+ve mode): m/z=323.15 [M+H]⁺; ¹H NMR (300 MHz, CDCl₃) δ 7.82 (m, 1H, ArH), 7.53 (m, 1H, ArH), 7.18 (m, 3H, 3×ArH), 3.10 (m, 2H, CH₂), 2.95 (m, 2H, CH₂), 2.68 (d, 12.0 Hz, ³J_{(H-P)}=10.2 Hz, 2×PNMe), 2.63 (s, 6H, 2×NMe); ¹³C NMR (75.5 MHz, CDCl₃) δ 138.1 (d, ³J_{(C-P)}=4.3 Hz), 130.3 (d, ²J_{(C-P)}=8.2 Hz), 125.9 (d, ²J_{(C-P)}=5.7 Hz), 123.6, 121.5, 118.6, 115.8 (d, ³J_{(C-P)}=7.2 Hz), 114.6, 58.6, 44.2, 36.7 (d, ²J_{(C-P)}=4.2 Hz), 22.2; ³¹P NMR (121.5 MHz, CDCl₃) δ 14.56.

### Example 1-50: 2-(1-di(dimethylamino)phosphoryl-5-methoxy-indol-3-yl)-N,N-dimethyl-ethanamine (Compound 558)

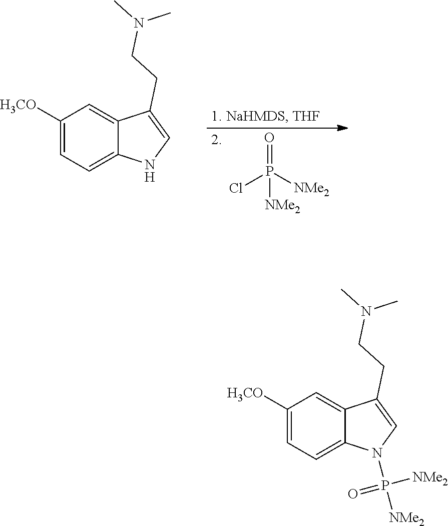 [SMILES: COc1ccc2[nH]cc(CCN(C)C)c2c1 ; CN(C)P(=O)(Cl)N(C)C ; COc1ccc2c(c1)c(CCN(C)C)cn2P(=O)(N(C)C)N(C)C]

NaHMDS, 1M in THF (0.96 mL, 0.96 mmol) was added to a stirred mixture of 5-OMe-DMT (200 mg, 0.92 mmol) in anhydrous THF (5 mL) at −78° C. After 30 min, N,N,N,N-tetramethylphosphorodiaminic chloride (157 mg, 0.14 mL, 0.92 mmol) was added and the mixture was warmed to rt and stirred for 16 h. The solvent was removed under vacuum and the residue was purified by column chromatography on silica gel eluting with a gradient of MeOH in DCM to afford Compound 558 (157 mg, 48%) as an oil. TLC: R_{f}=0.34 (DCM-MeOH, 8:2 v/v); LC-MS (+ve mode): m/z=353.15 [M+H]⁺; ¹H NMR (300 MHz, CDCl₃) δ 7.70 (d, J=9.0, 1H, ArH), 7.02 (m, 2H, 2×ArH), 6.85 (dd, J=9.0, 2.4 Hz, 1H, ArH), 3.83 (s, 3H, OMe), 2.96 (m, 2H, CH₂), 2.77 (m, 2H, CH₂), 2.67 (s, 6H, 2×PNMe), 2.63 (s, 6H, 2×PNMe), 2.45 (s, 6H, 2×NMe); ¹³C NMR (75.5 MHz, CDCl₃) δ 155.2, 132.9 (d, ³J_{(C-P)}=4.3 Hz), 131.3 (d, ²J_{(C-P)}=8.3 Hz), 126.3 (d, ²J_{(C-P)}=5.7 Hz), 116.7 (d, ³J_{(C-P)}=7.3 Hz), 115.3, 112.8, 101.1, 59.1, 55.9, 44.8, 36.7 (d, ²J_{(C-P)}=4.2 Hz), 23.0; ³¹P NMR (121.5 MHz, CDCl₃) δ 14.64.

### Example 1-51: bis(3-(2-(Dimethylamino)ethyl)-1H-indol-1-yl)methanone di-formate (Compound 170)

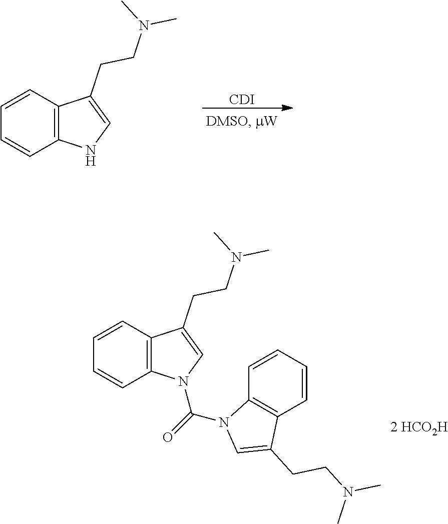 [SMILES: CN(C)CCc1c[nH]c2ccccc12 ; "CDI" ; CN(C)CCc1cn(C(=O)n2cc(CCN(C)C)c3ccccc32)c2ccccc12]

To a mixture of N,N-dimethyltryptamine (162 mg, 0.86 mmol) in DMSO (1.5 mL) was added carbonyldiimidazole (68 mg, 0.42 mmol) and the mixture was heated to 120° C. under microwave irradiation and stirred for 2 h. The mixture was quenched with saturated aqueous NaHCO₃ (10 mL) and extracted with EtOAc (3×25 mL). The combined organic layers were washed with H₂O (20 mL), saturated brine (20 mL), dried (MgSO₄) and concentrated under reduced pressure. This material was purified by reversed-phase chromatography, eluting with 0 to 100% acetonitrile in 0.1% formic acid to afford Compound 170 (48 mg, 35%) as an oil. LC-MS (+ve mode): m/z=403.25 [M+H]⁺; ¹H NMR (300 MHz, MeCN-d₃) δ 8.43 (s, 2H, 2×HCO), 7.99 (m, 2H, 2×ArH), 7.74 (m, 2H, 2×ArH), 7.53 (s, 2H, 2×ArH), 7.38 (m, 4H, 4×ArH), 3.06 (m, 4H, 2×CH₂), 2.96 (m, 4H, 2×CH₂), 2.50 (s, 12H, 4×NMe).

### Example 1-52: bis(3-(2-(Dimethylamino)ethyl)-5-methoxy-1H-indol-1-yl)methanone di-formate (Compound 169)

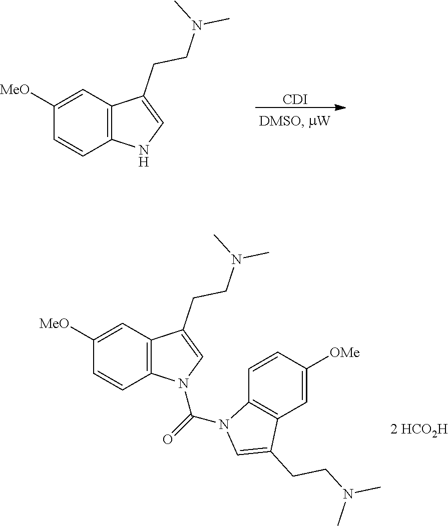 [SMILES: COc1ccc2[nH]cc(CCN(C)C)c2c1 ; "CDI" ; COc1ccc2c(c1)c(CCN(C)C)cn2C(=O)n1cc(CCN(C)C)c2cc(OC)ccc21]

To a solution of 5-methoxy-N,N-dimethyltryptamine (175 mg, 0.80 mmol) in DMSO (1.5 mL) was added CDI (63 mg, 0.39 mmol) and the mixture was heated to 120° C. under microwave irradiation and stirred for 2 h. The mixture was quenched with saturated aqueous NaHCO₃ (20 mL) and extracted with EtOAc (60 mL). The combined organic layers were washed with H₂O (20 mL), saturated brine (20 mL), dried (MgSO₄), filtered and concentrated to give an oil (186 mg). This material was purified by reversed-phase chromatography, eluting with 0 to 100% acetonitrile in 0.1% formic acid to afford Compound 169 (75.9 mg, 35%) as a solid. LC-MS (+ve mode): m/z=463.25 [M+H]⁺; ¹H NMR (300 MHz, MeCN-d₃) δ 8.28 (s, 2H, 2×HCO), 7.89 (d, J=9.0 Hz, 2H, 2×ArH), 7.58 (s, 2H, 2×ArH), 7.24 (d, J=2.4 Hz, 2H, 2×ArH), 7.00 (dd, J=9.0, 2.4 Hz, 2H, 2×ArH), 3.91 (s, 6H, 2×OMe), 3.30 (m, 4H, 2×CH₂), 3.15 (m, 4H, 2×CH₂), 2.73 (s, 12H, 4×NMe).

### Example 1-53: (3-(2-(Dimethylamino)ethyl)-1H-indol-1-yl)methanol (Compound 559)

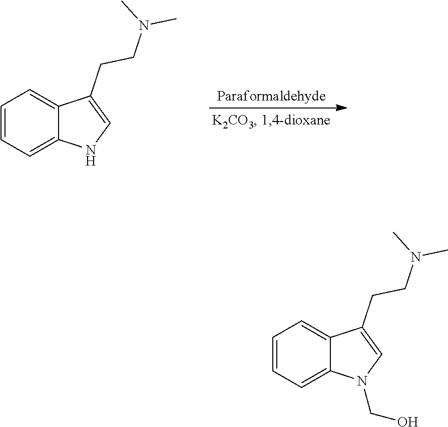 [SMILES: CN(C)CCc1c[nH]c2ccccc12 ; "Paraformaldehyde" ; CN(C)CCc1cn(CO)c2ccccc12]

To a mixture of DMT (188 mg, 1.0 mmol) in 1,4 dioxane (2 mL) was added K₂C₀₃ (414 mg, 3.0 mmol) and paraformaldehyde (90 mg, 3.0 mmol). The mixture was heated to 60° C. and stirred for 16 h, then diluted with DCM (15 mL) and filtered through Celite washing with DCM (2×10 mL). The filtrate was concentrated to afford Compound 559 (218 mg, 100%). LC-MS (+ve mode): m/z=219.10 [M+H]⁺; ¹H NMR (300 MHz, CD₃OD) δ 7.53 (m, 1H, ArH), 7.46 (m, 1H, ArH), 7.09 (m, 3H, 3×ArH), 5.51 (s, 2H, CH₂), 2.92 (m 2H, CH₂), 2.64 (m, 2H, CH₂) 2.33 (s, 6H, 2×N Me); ¹³C NMR (75 MHz, CD₃OD) δ 136.3, 128.6, 124.9, 121.4, 119.0, 118.2, 113.0, 109.4, 68.3, 66.7, 59.8, 44.0, 22.7.

### Example 1-54: (3-(2-(Dimethylamino)ethyl)-5-methoxy-1H-indol-1-yl)methanol (Compound 560)

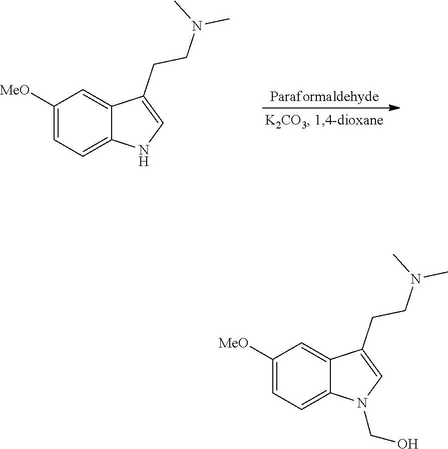 [SMILES: COc1ccc2[nH]cc(CCN(C)C)c2c1 ; "Paraformaldehyde" ; COc1ccc2c(c1)c(CCN(C)C)cn2CO]

To a solution of 5-OMe-DMT (218 mg, 1.0 mmol) in 1,4 dioxane (2 mL) was added K₂C₀₃ (414 mg, 3.0 mmol) and paraformaldehyde (90 mg, 3.0 mmol). The mixture was heated to 60° C. and stirred for 16 h, then diluted with DCM (15 mL) and filtered through Celite, washing with DCM (2×10 mL). The filtrate was concentrated to afford Compound 560 (190 mg, 76%) as an oil. LC-MS (+ve mode): m/z=249.15 [M+H]⁺; ¹H NMR (300 MHz, CD₃OD) δ 7.35 (d, J=8.9 Hz, 1H, Ar—H), 7.07 (s, 1H, ArH), 7.02 (d, J=2.4 Hz, 1H ArH), 6.83 (dd, ArH, J=8.9, 2.7 Hz, 1H), 5.47 (s, 2H, CH₂), 3.82 (s, 3H, OMe), 2.89 (m, 2H, CH₂), 2.64 (m, 2H, CH₂), 2.35 (s, 6H, 2×NMe); ¹³C NMR (75.5 MHz, CD₃OD) δ 154.2, 131.6, 125.6, 112.6, 111.3, 110.2, 100.3, 68.5, 59.6, 54.9, 44.0, 22.7.

### Example 1-55: (3-(2-(dimethylamino)ethyl)-1H-indol-1-yl)methyl pivalate (Compound 187)

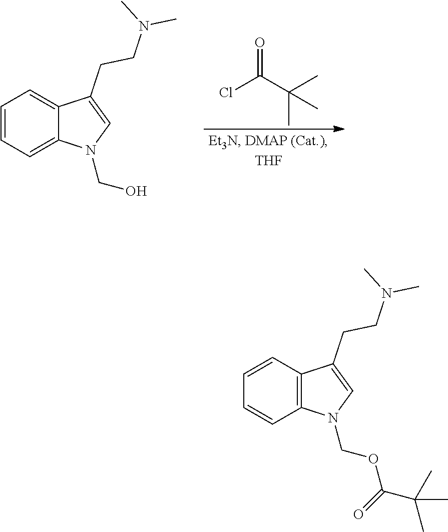

To (3-(2-(dimethylamino)ethyl)-1H-indol-1-yl)methanol (Compound 559, 109 mg, 0.5 mmol) in DCM (5 mL) at rt was added pivaloyl chloride (180 mg, 183 μL, 1.5 mmol), Et₃N (228 mg, 247 μL, 2.25 mmol) and DMAP (10 mg, 0.13 mmol). The mixture was stirred at rt for 16 h, then concentrated under vacuum and the crude residue was purified by column chromatography on silica gel, eluting with a gradient of MeOH in EtOAc to afford Compound 187 (91 mg, 60%) as an oil. LC-MS (+ve mode): m/z=303.10 [M+H]⁺; ¹H NMR (300 MHz, CD₃OD) δ 7.57 (m, 1H, ArH), 7.51 (m, 1H, ArH), 7.17 (m, 3H, 3×ArH), 6.15 (s, 2H, CH₂), 2.98 (m 2H, CH₂), 2.76 (m, 2H, CH₂), 2.43 (s, 6H, 2×NMe), 1.13 (s, 9H, ^{t}Bu); ¹³C NMR (75 MHz, CD₃OD) δ 179.5, 138.1, 130.0, 127.3, 123.5, 121.3, 119.7, 115.0, 110.9, 69.9, 60.6, 45.1, 39.9, 28.6, 27.3, 23.6.

### Example 1-56: (3-(2-(Dimethylamino)ethyl)-5-methoxy-1H-indol-1-yl)methyl pivalate (Compound 188)

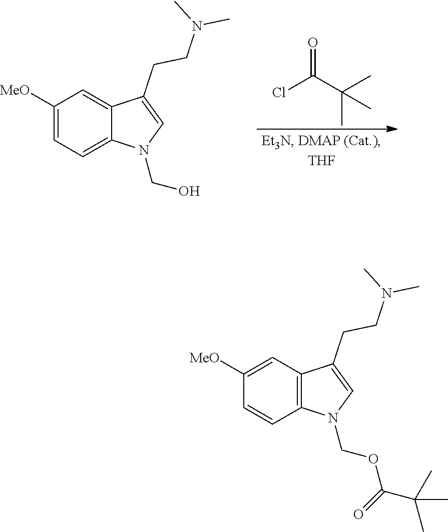

To (3-(2-(dimethylamino)ethyl)-5-methoxy-1H-indol-1-yl)methanol (Compound 560, 95 mg, 0.38 mmol.) in DCM (5 mL) at rt was added pivaloyl chloride (44.6 mg, 46 μL, 0.38 mmol), Et₃N (115 mg, 106 μL, 1.14 mmol) and DMAP (10 mg, 0.13 mmol). The mixture was stirred at rt for 16 h, then concentrated under vacuum and the residue was purified by column chromatography on silica gel, eluting with a gradient of MeOH in EtOAc to afford Compound 188 (30 mg, 23%) as an oil. LC-MS (+ve mode): m/z=333.15 [M+H]⁺; ¹H NMR (300 MHz, CD₃OD) δ 7.40 (d, J=8.8 Hz, 1H, ArH), 7.15 (s, 1H, ArH), 7.06 (d, J=2.4 Hz, 1H, ArH), 6.88 (dd, J=8.8, 2.4 Hz, 1H), 6.10 (s, 2H, CH₂), 3.85 (s, 3H, OMe), 2.92 (m 2H, CH₂), 2.71 (m, 2H, CH₂) 2.40 (s, 6H, 2×NMe) 1.12 (s, 9H, ^{t}Bu); ¹³C NMR (75 MHz, CD₃OD) δ 154.8, 126.7, 126.5, 111.9, 111.7, 110.5, 110.3, 100.6, 100.4, 68.6, 59.0, 58.8, 54.9, 43.4, 27.0, 25.9, 22.0.

### Example 1-57: (3-(2-(Dimethylamino)ethyl)-1H-indol-1-yl)methyl ethyl carbonate (Compound 561)

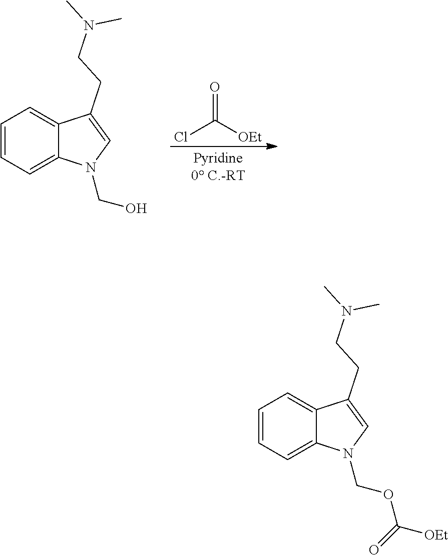 [SMILES: CN(C)CCc1cn(CO)c2ccccc12 ; CCOC(=O)Cl ; CCOC(=O)OCn1cc(CCN(C)C)c2ccccc21]

To (3-(2-(dimethylamino)ethyl)-5-methoxy-1H-indol-1-yl)methanol (Compound 559, 218 mg, 1.0 mmol) in anhydrous pyridine (5 mL) at 0° C. under an atmosphere of N2 was added ethyl chloroformate (119 mg, 105 μL, 1.1 mmol) dropwise. The mixture was slowly warmed to rt and stirred for 1 h, then concentrated under vacuum and EtOAc (50 mL) and NaHCO₃ (25 mL) added. The phases were separated, and the organic phase was washed with H₂O (25 mL), brine (25 mL), dried (MgSO₄), filtered and the filtrate was concentrated to afford Compound 561 (169 mg) as an oil. LC-MS (+ve mode): m/z=291.15 [M+H]⁺.

### Example 1-58: (3-(2-(Dimethylamino)ethyl)-5-methoxy-1H-indol-1-yl)methyl ethyl carbonate (Compound 562)

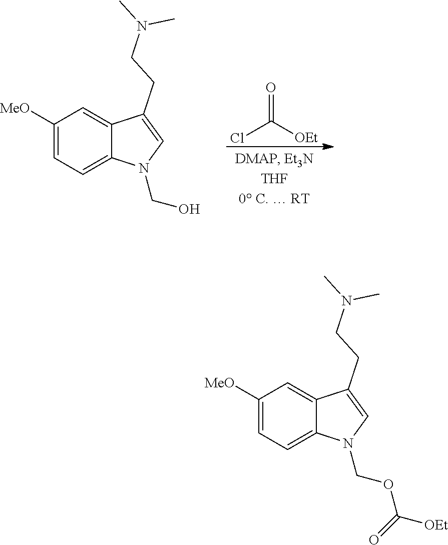 [SMILES: COc1ccc2c(c1)c(CCN(C)C)cn2CO ; CCOC(=O)Cl ; CCOC(=O)OCn1cc(CCN(C)C)c2cc(OC)ccc21]

To (3-(2-(dimethylamino)ethyl)-1H-indol-1-yl)methanol (Compound 560, 125 mg, 0.5 mmol), DMAP (2 mg, 15 μmol) and trimethylamine (101 mg, 139 μmol) in anhydrous THF (5 mL) at 0° C. under an atmosphere of N2 was added a solution of ethyl chloroformate (70 mg, 60 μL, 0.65 mmol) in anhydrous THF (0.4 mL) dropwise. The mixture was stirred at 0° C. for 1 h, then slowly warmed to rt and stirred for 18 h. Additional DMAP (24 mg, 180 μmol) and triethylamine (101 mg, 139 μmol) were added followed by a solution of ethyl chloroformate (109 mg, 96 μmol, 1.0 mmol) in anhydrous THF (1 mL). The reaction mixture was stirred at rt for an additional 24 h, then concentrated under reduced pressure to afford Compound 562 (189 mg) as an oil. LC-MS (+ve mode): m/z=343.10 [M+Na]⁺.

### Example 1-59: Di-tert-butyl ((3-(2-(dimethylamino)ethyl)-1H-indol-1-yl)methyl) phosphate (Compound 264)

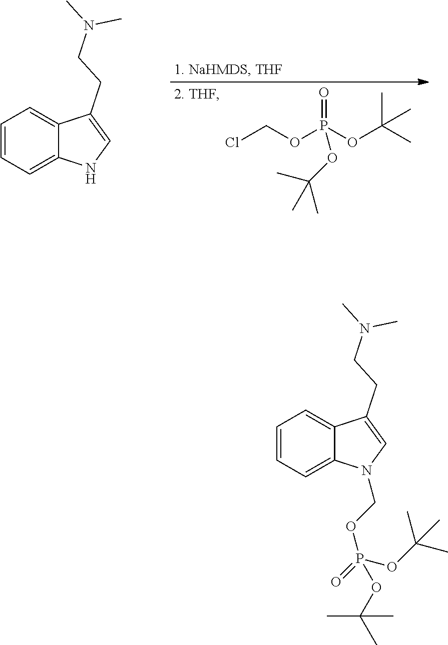 [SMILES: CN(C)CCc1c[nH]c2ccccc12 ; CC(C)(C)OP(=O)(OCCl)OC(C)(C)C ; CN(C)CCc1cn(COP(=O)(OC(C)(C)C)OC(C)(C)C)c2ccccc12]

NaHMDS, 1M in THF (1.4 mL, 1.4 mmol) was added to a stirred solution of DMT (250 mg, 1.33 mmol) in anhydrous THF (18 mL) at −78° C. After 30 min, the mixture was added dropwise to di-tert-butyl chloromethyl phosphate (310 mg, 1.20 mmol), the mixture was warmed to rt and stirred for 16 h, then concentrated to dryness and dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated under vacuum. The crude residue was purified by column chromatography on silica gel, eluting with MeOH in DCM, followed by reversed-phase chromatography, eluting with MeCN in H₂O and subsequently purification using a Biotage® KP-Amino D column, eluting with a mixture of PE in EtOAc to MeOH in EtOAc to afford Compound 264 (123 mg). LC-MS (+ve mode): m/z=411.20 [M+H]⁺.

### Example 1-60: Di-tert-butyl ((3-(2-(dimethylamino)ethyl)-5-methoxy-1H-indol-1-yl)methyl) phosphate (Compound 256)

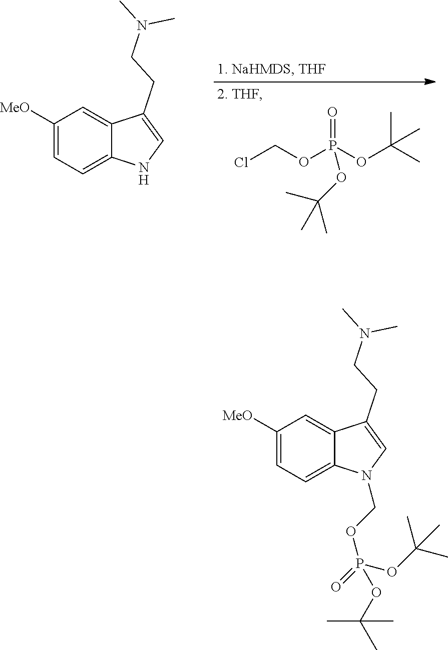 [SMILES: COc1ccc2[nH]cc(CCN(C)C)c2c1 ; CC(C)(C)OP(=O)(OCCl)OC(C)(C)C ; COc1ccc2c(c1)c(CCN(C)C)cn2COP(=O)(OC(C)(C)C)OC(C)(C)C]

NaHMDS, 1M in THF (1.4 mL, 1.4 mmol) was added to a stirred solution of 5-OMe-DMT (150 mg, 0.69 mmol) in anhydrous THF (9 mL) at −78° C. After 30 min, the resulting mixture was added dropwise to di-tert-butyl chloromethyl phosphate (160 mg, 0.62 mmol), the mixture was warmed to rt and stirring was continued for 16 h at rt. The mixture was concentrated to dryness, then dissolved into a mixture of DCM (20 mL) and NaHCO₃ (20 mL). The phases were separated, and the organic phase washed with H₂O (2×20 mL), brine (20 mL), dried (MgSO₄), filtered and the filtrate was concentrated under vacuum. The residue was purified by column chromatography on silica gel, eluting with MeOH in DCM, followed by reversed-phase chromatography, eluting with MeCN in H₂O and subsequently purification using a Biotage® KP-Amino D column, eluting with a mixture of PE to EtOAc to MeOH in EtOAc to afford Compound 256 (74 mg). LC-MS (+ve mode): m/z=441.20 [M+H]⁺.

### Example 1-61: 1-(((S)-2-amino-3-methylbutanoyl)oxy)-2-methylpropyl 3-(2-(dimethylamino)ethyl)-5-methoxy-1H-indole-1-carboxylate di-trifluoroacetate (Compound 563)

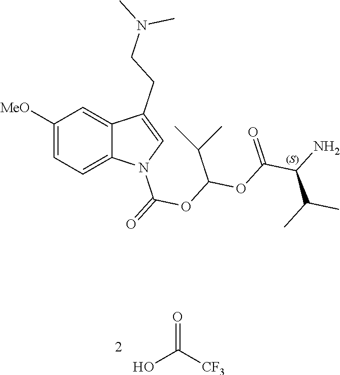

### Step 1: 1-Chloro-2-methylpropyl 3-(2-(dimethylamino)ethyl)-5-methoxy-1H-indole-1-carboxylate

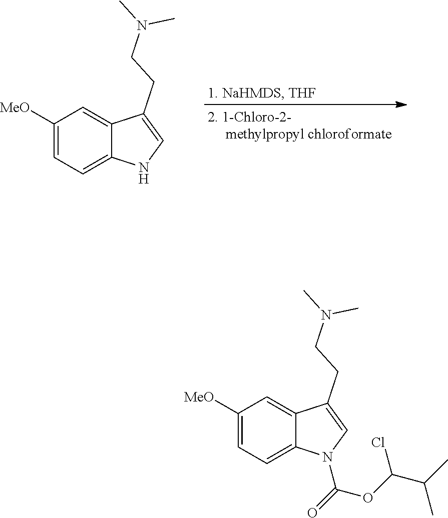 [SMILES: COc1ccc2[nH]cc(CCN(C)C)c2c1 ; "1. NaHMDS, THF" ; COc1ccc2c(c1)c(CCN(C)C)cn2C(=O)OC(Cl)C(C)C]

NaHMDS, 1M in THF (3.25 mL, 3.25 mmol) was added to a stirred solution of 5-OMe-DMT (355 mg, 1.63 mmol) in anhydrous THF (16 mL) at −78° C. After 30 min, 1-chloro-2-methylpropyl chloroformate (556 mg, 474 μL, 3.25 mmol) was added dropwise and stirring was continued for 30 min at −78° C., then allowed to warm to rt and stirred for 2 h. The mixture was quenched with H₂O (10 mL) and concentrated to dryness and the residual material was dissolved in a mixture of DCM (15 mL) and H₂O (15 mL). The phases were separated, and the organic phase was washed with H₂O (2×15 mL), sat. brine (20 mL), dried (Na₂SO₄), filtered and the filtrate was concentrated to give a semi-solid. The material was purified by column chromatography on silica gel, eluting with a gradient of MeOH in EtOAc containing 0.1% Et₃N to give the product (154 mg, 27%) as an oil. LC-MS (+ve mode): m/z=343.10 & 345.10 [M+H]⁺; ¹H NMR (300 MHz, CDCl₃) δ 8.00 (br, 1H, ArH), 7.29 (s, 1H, ArH), 6.95 (d, J=2.5 Hz, 1H, ArH), 6.89 (dd, J=8.9, 2.5 Hz, 1H, ArH), 6.47 (d, J=4.6 Hz, 1H, CH), 3.81 (s, 3H, OCH₃), 2.80 (m, 2H, CH₂), 2.61 (m, 2H, CH₂), 2.31 (s, 6H, 2×NCH₃), 1.11 (dd, J=6.8, 4.5 Hz, 6H, 2×CH₃).

### Step 2: N-(tert-Butoxycarbonyl)-L-valinate cesium salt

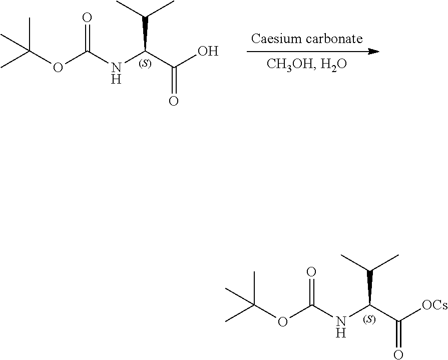

N-(tert-Butoxycarbonyl)-L-valine (1.24 g, 5.70 mmol) was dissolved in MeOH (24 mL) and H₂O (2.4 mL). A 20% w/w aqueous solution of Cs₂CO₃ was added dropwise until pH 7 was achieved. The solution was concentrated in vacuo to give a clear residue, which was lyophilised to give N-(tert-butoxycarbonyl)-L-valine cesium salt (1.99 g, quant) as a solid.

### Step 3: 1-(((S)-2-((tert-butoxycarbonyl)amino)-3-methylbutanoyl)oxy)-2-methylpropyl 3-(2-(dimethylamino)ethyl)-5-methoxy-1H-indole-1-carboxylate

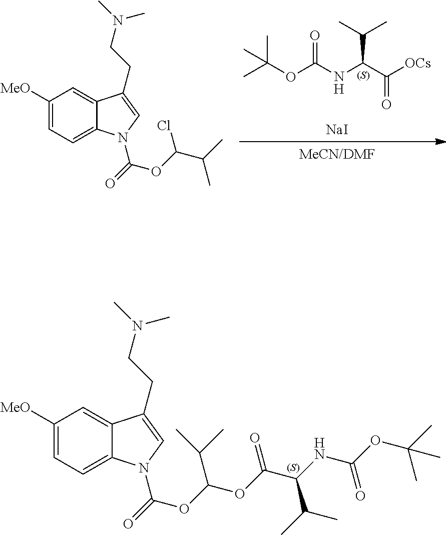

1-Chloro-2-methylpropyl 3-(2-(dimethylamino)ethyl)-5-methoxy-1H-indole-1-carboxylate (154 mg, 0.44 mmol) was dissolved in MeCN (8 mL), then N-(tert-butoxycarbonyl)-L-valinate cesium salt (236 mg, 0.68 mmol) and NaI (66 mg, 0.44 mmol) were added. The mixture was heated to 70° C. and stirred overnight. DMF (4 mL) was added and the mixture was stirred at 70° C. for a further 72 h. The mixture was concentrated under reduced pressure and the residue was purified twice by column chromatography on silica gel, eluting with a gradient of MeOH in EtOAc to give the product (77 mg) as a solid. LC-MS (+ve mode): m/z=534.30 [M+H]⁺

### Step 4: 1-(((S)-2-amino-3-methylbutanoyl)oxy)-2-methylpropyl 3-(2-(dimethylamino)ethyl)-5-methoxy-1H-indole-1-carboxylate

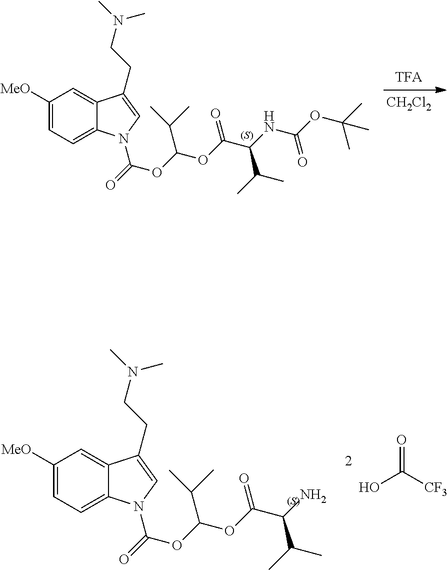

1-(((S)-2-((tert-butoxycarbonyl)amino)-3-methylbutanoyl)oxy)-2-methylpropyl 3-(2-(dimethylamino)ethyl)-5-methoxy-1H-indole-1-carboxylate (77 mg, 0.14 mmol) was dissolved in DCM (1.5 mL) at rt and TFA (0.82 g, 0.56 mL, 7.2 mmol) was added dropwise. The mixture was stirred at rt for 2 h, then concentrated under vacuum and the residue was purified by reversed-phase chromatography eluting with a gradient of acetonitrile in H₂O to afford Compound 563 (34.8 mg, 38%) as a semi-solid. LC-MS (+ve mode): m/z=434.20 [M+H]⁺; ¹H NMR (300 MHz, CD₃OD) δ 7.93 (d, J=8.6 Hz, 1H, ArH), 7.54 (s, 1H, ArH), 7.10 (d, J=2.3 Hz, 1H, ArH), 6.93 (m, 2H, ArH and CH), 4.02 (d, J=4.1 Hz, 1H, CH), 3.80 (s, 3H, OCH₃), 3.41 (m, 2H, CH₂), 3.10 (m, 2H, CH₂), 2.91 (s, 6H, 2×NCH₃), 2.23 (m, 1H, CH), 1.08 (dd, J=6.8, 4.9 Hz, 6H, 2×CH₃), 0.98 (dd, J=7.0, 4.3 Hz, 6H, 2×CH₃).

### Example 1-62: 1-(((S)-2-amino-3-methylbutanoyl)oxy)-2-methylpropyl 3-(2-(dimethylamino)ethyl)-1H-indole-1-carboxylate di-trifluoroacetate (Compound 564)

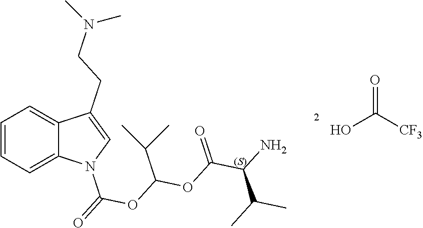

### Step 1: 1-Chloro-2-methylpropyl 3-(2-(dimethylamino)ethyl)-1H-indole-1-carboxylate

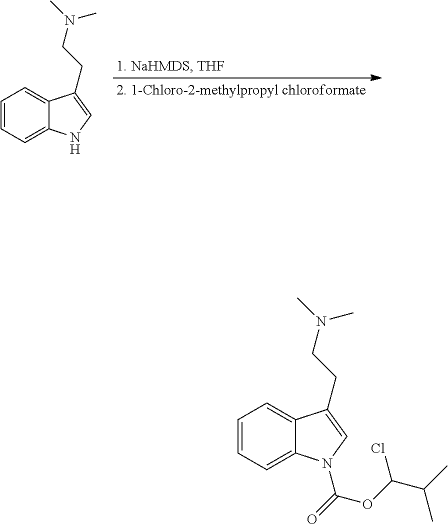

NaHMDS, 1M in THF (3.06 mL, 3.06 mmol) was added to a stirred solution of DMT (288 mg, 1.53 mmol) in anhydrous THF (15 mL) at −78° C. After 30 min, 1-chloro-2-methylpropyl chloroformate (523 mg, 446 μL, 3.06 mmol) was added dropwise and stirring was continued at −78° C. for 30 min, then allowed to warm to rt and stirred for 2.5 h. The mixture was quenched with H₂O (10 mL), then concentrated to dryness and dissolved in a mixture of DCM (15 mL) and H₂O (15 mL). The phases were separated and the organic phase was washed with H₂O (2×15 mL), sat. brine (20 mL), dried (Na₂SO₄) and concentrated under vacuum. The residue was purified by column chromatography on silica gel, eluting with a gradient of MeOH in EtOAC to give the product (98 mg, 20%) as a semi-solid. LC-MS (+ve mode): m/z=323.10 [M+H]⁺; ¹H NMR (300 MHz, CDCl₃) δ 8.13 (br, 1H, ArH), 7.60 (d, J=7.6 Hz, 1H, ArH), 7.41 (s, 1H, ArH), 7.31 (m, 2H, 2×ArH), 6.48 (d, J=4.6 Hz, 1H, CH), 3.32 (m, 2H, CH₂), 3.22 (m, 2H, CH₂), 2.82 (s, 6H, 2×NCH₃), 2.31 (m, 1H, CH), 1.13 (dd, J=6.8, 5.1 Hz, 6H, 2×CH₃).

### Step 2: 1-(((S)-2-((tert-butoxycarbonyl)amino)-3-methylbutanoyl)oxy)-2-methylpropyl 3-(2-(dimethylamino)ethyl)-1H-indole-1-carboxylate

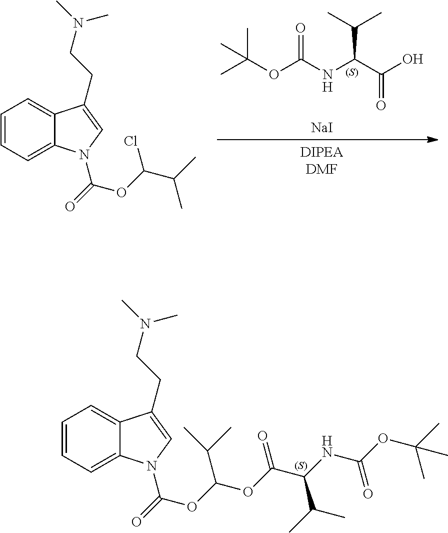

1-Chloro-2-methylpropyl 3-(2-(dimethylamino)ethyl)-1H-indole-1-carboxylate (98 mg, 0.30 mmol) was dissolved in DMF (6 mL), then N-(tert-butoxycarbonyl)-L-valine (132 mg, 0.61 mmol), N,N-diisopropylethylamine (196 mg, 265 μL, 1.52 mmol) and NaI (46 mg, 0.30 mmol) were added. The mixture was heated to 60° C. and stirred for 4 h, then heated to 70° C. and stirred for a further 96 h. The sample was concentrated under vacuum and the residue was purified by column chromatography on silica gel, eluting with a gradient of MeOH in EtOAc to give the product (78 mg) as a semi-solid. LC-MS (+ve mode): m/z=504.30 [M+H]⁺.

### Step 3: 1-(((S)-2-amino-3-methylbutanoyl)oxy)-2-methylpropyl 3-(2-(dimethylamino)ethyl)-1H-indole-1-carboxylate di-trifluoroacetate

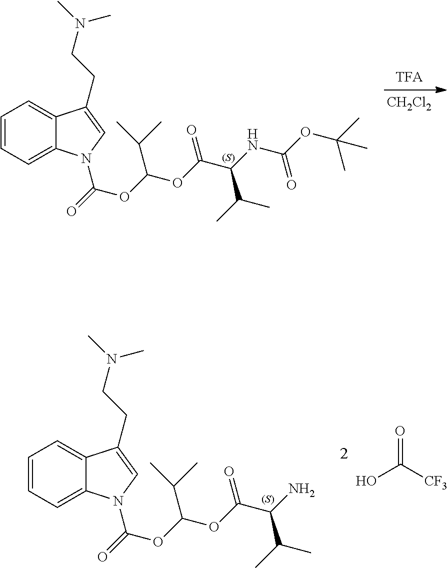

1-(((S)-2-((tert-butoxycarbonyl)amino)-3-methylbutanoyl)oxy)-2-methylpropyl 3-(2-(dimethylamino)ethyl)-1H-indole-1-carboxylate (78 mg, 0.16 mmol) was dissolved in DCM (1.6 mL) at rt and TFA (0.88 g, 0.6 mL, 7.7 mmol) was added dropwise. The mixture was stirred at rt for 1.5 h, then concentrated under vacuum and the residue was purified by reverse phase chromatography eluting with a gradient of acetonitrile in H₂O to afford Compound 564 (15.2 mg) as a semi-solid. LC-MS (+ve mode): m/z=404.25 [M+H]⁺; ¹H NMR (300 MHz, CD₃OD) δ 8.04 (m, 1H, ArH), 7.59 (m, 2H, ArH), 7.28 (m, 2H, ArH), 6.92 (d, J=4.8 Hz, 1H, CH), 4.01 (d, J=4.1 Hz, 1H, CH), 3.41 (m, 2H, CH₂), 3.11 (dd, J=9.8, 6.3 Hz, 2H, CH₂), 2.89 (s, 6H, 2×NCH₃), 2.24 (m, 2H, 2×CH), 1.38 (m, 6H, 2×CH₃), 0.96 (dd, J=7.0, 4.0 Hz, 6H, 2×CH₃).

### Example 1-63: tert-Butyl (((3-(2-(dimethylamino)ethyl)-5-methoxy-1H-indole-1-carbonyl)oxy)methyl) succinate (Compound 565)

### Cesium 4-(tert-butoxy)-4-oxobutanoate

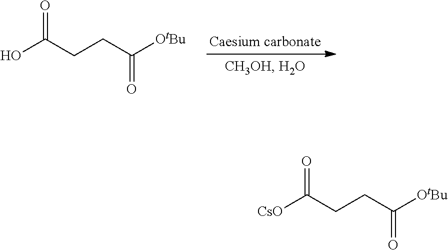

4-(tert-Butoxy)-4-oxobutanoic acid (0.50 g, 2.88 mmol) was dissolved in MeOH (12 mL) and H₂O (1.2 mL). A 20% w/w aqueous solution of Cs₂CO₃ was added dropwise until pH 7 was achieved. The mixture was concentrated in vacuo to give a clear residue, which was lyophilised overnight to give the product (0.88 g, quant) as a solid.

### Chloromethyl 3-(2-(dimethylamino)ethyl)-5-methoxy-1H-indole-1-carboxylate

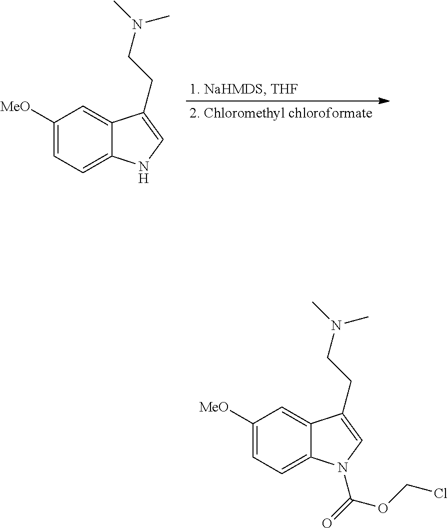

NaHMDS, 1M in THF (1.8 mL, 1.8 mmol) was added to a stirred solution of 5-OMe-DMT (200 mg, 0.92 mmol) in anhydrous THF (13 mL) at −78° C. After 30 min, chloromethyl chloroformate (236 mg, 163 μL, 1.83 mmol) was added dropwise, the mixture was allowed to warm to rt and stirring was continued for 20 h. The mixture was concentrated to dryness and the residue was purified by column chromatography on silica gel, eluting with a gradient of MeOH in EtOAc to give the product (261 mg, 91%) as a semi-solid, containing ˜15% of 5-OMe-DMT. LC-MS (+ve mode): m/z=311.05 and 313.05 [M+H]⁺.

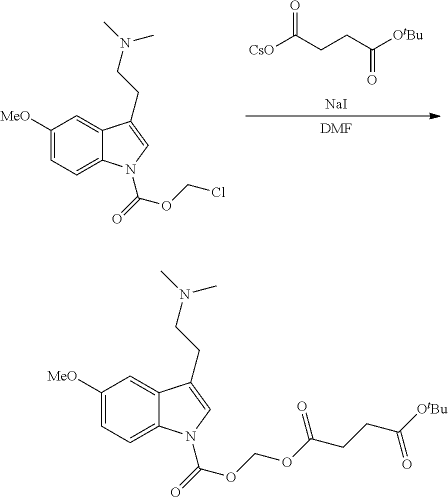

Chloromethyl 3-(2-(dimethylamino)ethyl)-5-methoxy-1H-indole-1-carboxylate (205 mg, 0.66 mmol) was dissolved in DMF (4 mL), then cesium 4-(tert-butoxy)-4-oxobutanoate (202 mg, 0.66 mmol) and NaI (99 mg, 0.66 mmol) were added. The mixture was heated to 60° C. and stirred overnight, then concentrated under vacuum. The residue was purified by column chromatography on silica gel, eluting with a gradient of PE and MeOH in EtOAc to afford Compound 565 (86 mg) as an oil. This was used without further purification. LC-MS (+ve mode): m/z=449.20 [M+H]⁺.

### Example 1-63: 4-(((3-(2-(Dimethylamino)ethyl)-5-methoxy-1H-indole-1-carbonyl)oxy)methoxy)-4-oxobutanoic acid (Compound 566)

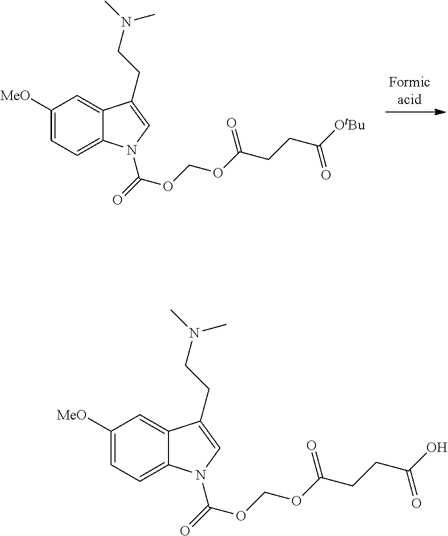 [SMILES: COc1ccc2c(c1)c(CCN(C)C)cn2C(=O)OCOC(=O)CCC(=O)OC(C)(C)C ; COc1ccc2c(c1)c(CCN(C)C)cn2C(=O)OCOC(=O)CCC(=O)O]

tert-Butyl (((3-(2-(dimethylamino)ethyl)-5-methoxy-1H-indole-1-carbonyl)oxy)methyl) succinate (Compound 564, 19 mg, 0.04 mmol) was stirred in formic acid (0.5 mL) for 2 h, then concentrated under vacuum to afford Compound 566 (17 mg) as a solid. LC-MS (+ve mode): m/z=393.15 [M+H]⁺.

### Example 1-64: 5-(((3-(2-(Dimethylamino)ethyl)-5-methoxy-1H-indole-1-carbonyl)oxy)methoxy)-5-oxopentanoic acid (Compound 567)

### Cesium 5-(tert-butoxy)-5-oxopentanoate

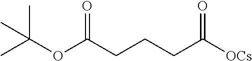

To a solution of pentanedioc acid mono-tert-butyl ester (300 mg, 1.59 mmol) in MeOH (4.40 mL) and H₂O (0.44 mL) was added a 20% w/w aqueous solution of Cs₂CO₃ until pH 7 was achieved. The mixture was concentrated, azeotroping with MeCN (2×10 mL) to give the product as a semi-solid.

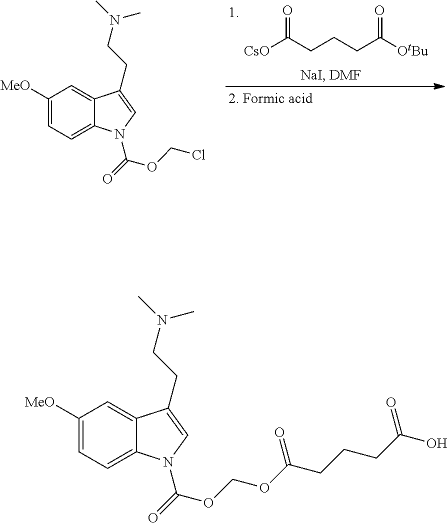

Chloromethyl 3-(2-(dimethylamino)ethyl)-5-methoxy-1H-indole-1-carboxylate formate (50 mg, 0.16 mmol) was dissolved in DMF (1 mL), then cesium 5-(tert-butoxy)-5-oxopentanoate (52 mg, 0.16 mmol) and NaI (24 mg, 0.16 mmol) were added. The mixture was heated to 60° C. and stirred overnight, then concentrated under vacuum to give a solid. The solid was stirred in formic acid (1 mL) for 2 h, then concentrated under vacuum to afford Compound 567 (150 mg) as a solid. LC-MS (+ve mode): m/z=407.15 [M+H]⁺.

### Example 1-65: 6-(((3-(2-(Dimethylamino)ethyl)-5-methoxy-1H-indole-1-carbonyl)oxy)methoxy)-6-oxohexanoic acid (Compound 568)

### Cesium 6-(tert-butoxy)-6-oxohexanoate

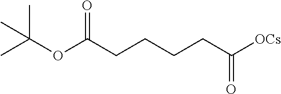

To a solution of 6-(tert-butoxy)-6-oxohexanoic acid (375 mg, 1.85 mmol) in MeOH (7.70 mL) and H₂O (0.77 mL) was added a 20% w/w aqueous solution of was added dropwise until pH 7 was achieved. The reaction mixture was concentrated, azeotroping with MeCN (2×10 mL) to give the product as a semi-solid.

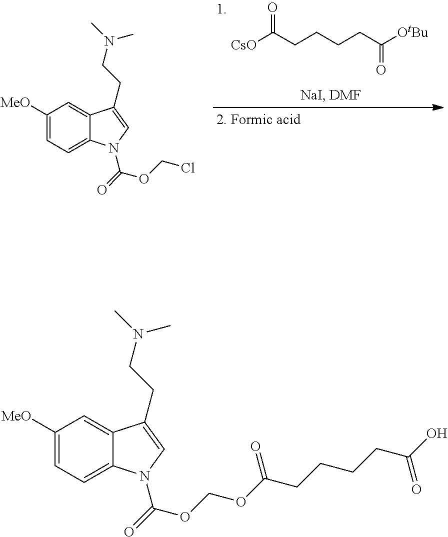

Chloromethyl 3-(2-(dimethylamino)ethyl)-5-methoxy-1H-indole-1-carboxylate formate salt (50 mg, 0.16 mmol) was dissolved in DMF (1 mL), then cesium 6-(tert-butoxy)-6-oxohexanoate (52 mg, 0.16 mmol) and NaI (24 mg, 0.16 mmol) were added. The mixture was heated to 60° C. and stirred overnight, then concentrated under vacuum to give a solid. The solid was stirred in formic acid (1 mL) for 2 h, then concentrated under vacuum to afford Compound 568 (74 mg) as a solid. LC-MS (+ve mode): m/z=421.15 [M+H]⁺.

### Example 1-66: Chloromethyl 3-(2-(dimethylamino)ethyl)-1H-indole-1-carboxylate (Compound 569)

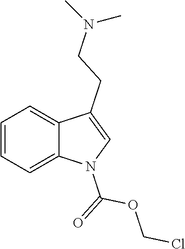 [SMILES: CN(C)CCc1cn(C(=O)OCCl)c2ccccc12]

To a solution of DMT (1.00 g, 5.3 mmol) in anhydrous tetrahydrofuran (60 mL) at −78° C. under an atmosphere of N2 was added NaHMDS, 1M in THF (10.6 mL, 10.6 mmol) and the mixture was stirred for 30 min at −78° C. Chloromethyl chloroformate (1.37 g, 0.94 mL, 10.6 mmol) was added dropwise, the mixture was stirred at −78° C. for 15 min and then warmed to rt and stirred for 2 h. H₂O (5 mL) was added, the mixture was concentrated under vacuum and the residue was purified by column chromatography on silica gel, eluting with 0 to 50% MeOH in EtOAc to afford Compound 569 (996 mg) as an oil. LC-MS (+ve mode): m/z=281.10 [M+H]⁺.

### Example 1-67: tert-Butyl (((3-(2-(dimethylamino)ethyl)-1H-indole-1-carbonyl)oxy)methyl) glutarate (Compound 570)

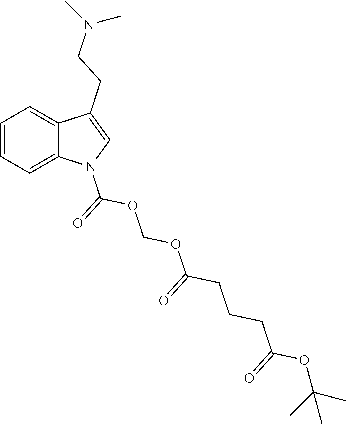 [SMILES: CN(C)CCc1cn(C(=O)OCOC(=O)CCCC(=O)OC(C)(C)C)c2ccccc12]

To a solution of chloromethyl 3-(2-(dimethylamino)ethyl)-1H-indole-1-carboxylate (80 mg, 0.29 mmol) in anhydrous DMF (0.5 mL) under an atmosphere of N2 was added NaI (43 mg, 0.29 mmol) and a solution of cesium 5-(tert-butoxy)-5-oxopentanoate (91 mg, 0.29 mmol) in anhydrous DMF (1 mL). The mixture was stirred at rt overnight, then heated to 80° C. and stirred for 2.5 h. The mixture was cooled to rt and concentrated to afford Compound 570 as an oil. LC-MS (+ve mode): m/z=433.20 [M+H]⁺.

### Example 1-68: 5-(((3-(2-(dimethylamino)ethyl)-1H-indole-1-carbonyl)oxy)methoxy)-5-oxopentanoic acid (Compound 571)

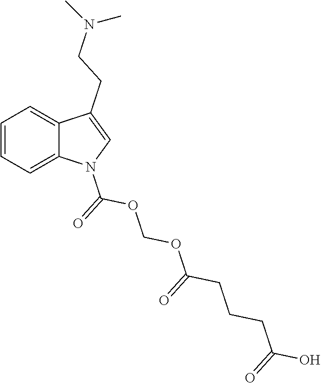 [SMILES: CN(C)CCc1cn(C(=O)OCOC(=O)CCCC(=O)O)c2ccccc12]

To tert-butyl (((3-(2-(dimethylamino)ethyl)-1H-indole-1-carbonyl)oxy)methyl) glutarate (Compound 570) was added formic acid (2 mL) and the mixture was stirred at rt for 1 h, then concentrated under vacuum to afford Compound 571. LC-MS (+ve mode): m/z=377.15 [M+H]⁺.

### Example 1-69: tert-Butyl (((3-(2-(dimethylamino)ethyl)-1H-indole-1-carbonyl)oxy)methyl) adipate (Compound 572)

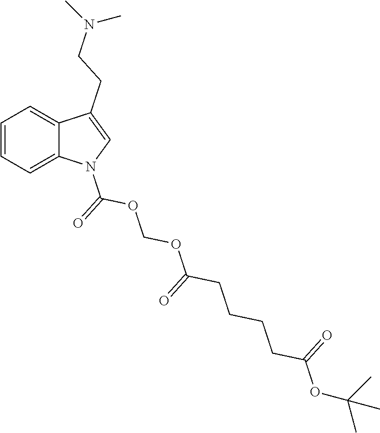 [SMILES: CN(C)CCc1cn(C(=O)OCOC(=O)CCCCC(=O)OC(C)(C)C)c2ccccc12]

To a solution of chloromethyl 3-(2-(dimethylamino)ethyl)-1H-indole-1-carboxylate (80 mg, 0.29 mmol) in anhydrous DMF (0.5 mL) under an atmosphere of N2 was added NaI (43 mg, 0.29 mmol) and a solution of cesium 6-(tert-butoxy)-6-oxohexanoate (91 mg, 0.29 mmol) in anhydrous DMF (1 mL). The mixture was stirred overnight at rt, then heated to 80° C. and stirred for 2.5 h. The mixture was cooled to rt and concentrated under vacuum to afford Compound 572 as an oil. LC-MS (+ve mode): m/z=447.20 [M+H]⁺.

### Example 1-70: 6-(((3-(2-(Dimethylamino)ethyl)-1H-indole-1-carbonyl)oxy)methoxy)-6-oxohexanoic acid (Compound 573)

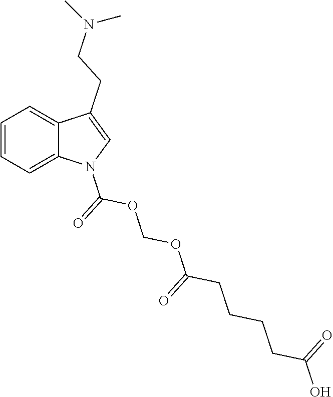 [SMILES: CN(C)CCc1cn(C(=O)OCOC(=O)CCCCC(=O)O)c2ccccc12]

To tert-butyl (((3-(2-(dimethylamino)ethyl)-1H-indole-1-carbonyl)oxy)methyl) adipate (Compound 572) was added formic acid (2 mL) and the reaction mixture was stirred at rt for 0.5 h. The mixture was concentrated under vacuum to afford Compound 573. LC-MS (+ve mode): m/z=391.20 [M+H]⁺.

### Example 1-71: Ethyl 3-(((3-(2-(dimethylamino)ethyl)-LH-indol-1-yl)sulfonyl)oxy)-2,2-dimethylpropanoate (Compound 457)

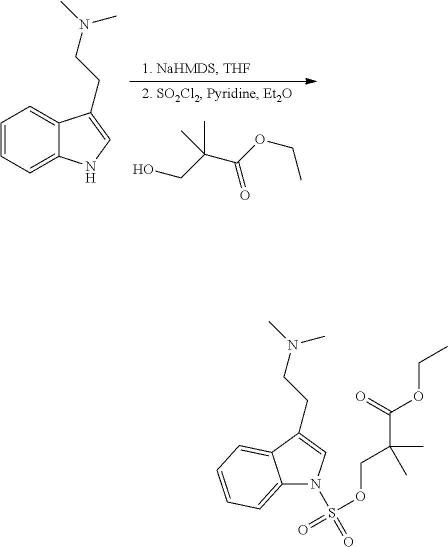

Sulfuryl chloride (143 mg, 86 μL, 1.06 mmol) in Et₂₀ (20 mL) was added dropwise to a solution of ethyl 3-hydroxy-2,2-dimethylpropanoate (254 mg, 1.74 mmol) and pyridine (84 mg, 86 μL, 1.06 mmol) in Et₂₀ (5 mL) at −78° C. and stirring was continued at −78° C. for 30 min, then filtered through Celite and the filtrate was concentrated under vacuum to give a colourless oil which was used directly in the next step.

NaHMDS, 1M in THF (1.12 mL, 1.12 mmol) was added to a solution of DMT (200 mg, 1.06 mmol) in anhydrous THF (5 mL) at −78° C. and stirring was continued at −78° C. 30 min, after which time a THF solution of ethyl 3-((chlorosulfonyl)oxy)-2,2-dimethylpropanoate (3 mL) was added and the mixture was warmed to rt and stirred for 72 h, then concentrated under vacuum to afford Compound 457 (512 mg) as a semi-solid. LC-MS (+ve mode): m/z=397.15 [M+H]⁺.

### Example 1-72: Ethyl 3-(((3-(2-(dimethylamino)ethyl)-5-methoxy-LH-indol-1-yl)sulfonyl)oxy)-2,2-dimethylpropanoate (Compound 433)

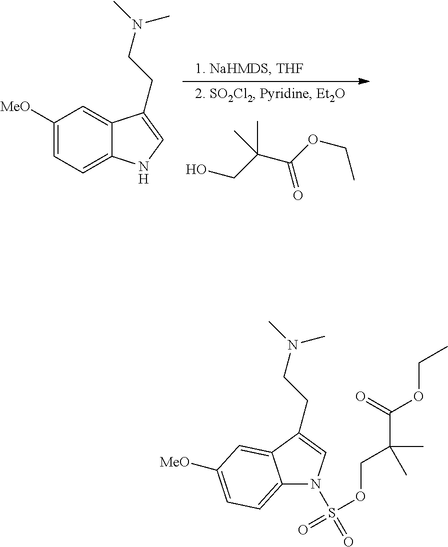

Sulfuryl chloride (143 mg, 86 μL, 1.06 mmol) in Et₂₀ (20 mL) was added dropwise to a solution of ethyl 3-hydroxy-2,2-dimethylpropanoate (254 mg, 1.74 mmol) and pyridine (84 mg, 86 μL, 1.06 mmol) in Et₂₀ (5 mL) at −78° C. and stirring was continued at −78° C. for 30 min, then filtered through Celite and the filtrate was concentrated under vacuum to give an oil, which was used directly in the next step.

NaHMDS, 1M in THF (1.12 mL, 1.12 mmol) was added to a solution of 5-OMe-DMT (231 mg, 1.06 mmol) in anhydrous THF (5 mL) at −78° C. and stirring was continued at −78° C. for 30 min, after which time a THF solution of ethyl 3-((chlorosulfonyl)oxy)-2,2-dimethylpropanoate (3 mL) was added. The mixture was warmed to rt and stirred for 72 h, then concentrated under vacuum to afford Compound 433 (574 mg) as a semi-solid. LC-MS (+ve mode): m/z=427.15 [M+H]⁺.

### Example 1-73: 4-(3-(2-(dimethylamino)ethyl)-1H-indol-1-yl)-2,2-dimethyl-4-oxobutanoic acid HCl salt (Compound 576)

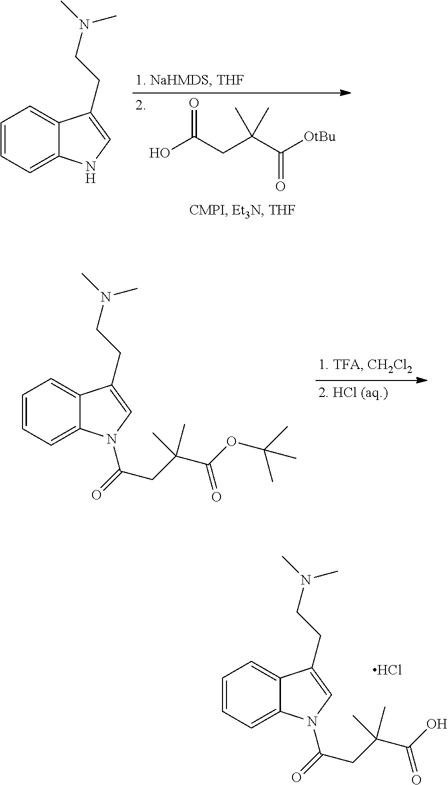 [SMILES: CN(C)CCc1c[nH]c2ccccc12 ; CC(C)(C)OC(=O)C(C)(C)CC(=O)O ; CN(C)CCc1cn(C(=O)CC(C)(C)C(=O)OC(C)(C)C)c2ccccc12 ; CN(C)CCc1cn(C(=O)CC(C)(C)C(=O)O)c2ccccc12 ; Cl]

NaHMDS, 1M in THF (2.23 mL, 2.23 mmol) was added to a stirred solution of DMT (400 mg, 2.12 mmol) in anhydrous THF (10 mL) at −78° C. In a separate vessel, 4-(tert-butoxy)-3,3-dimethyl-4-oxobutanoic acid (0.53 g, 2.12 mmol) and 2-chloro-1-methylpyridinium iodide (0.60 g, 2.34 mmol) were dissolved in anhydrous THF (10 mL). Et₃N (472 mg, 0.66 mL, 4.66 mmol) was added and the mixture was stirred at rt. After 30 min, the DMT solution was added and the mixture was stirred at rt for 16 h, then concentrated under vacuum and the residue was purified by column chromatography on silica gel, eluting with a gradient of MeOH in DCM to afford an oil (1.2 g, quant.). TLC: R_{f}=0.63 (DCM-MeOH, 8:2 v/v); LC-MS (+ve mode): m/z=373.20 [M+H]⁺.

The above material was dissolved in DCM (15 mL) and TFA (12.1 g, 8.2 mL, 106 mmol) was added at rt. The mixture was stirred at rt for 2 h, then concentrated under vacuum and azeotroped with CHCl₃ (3×20 mL) to give a dark residue, which was taken up in 1M HCl (3 mL) and purified by reversed-phase chromatography on silica eluting with a gradient of acetonitrile in 0.02% hydrochloric acid to afford Compound 576 (170 mg, 23% over 2 steps) as a solid. TLC: R_{f}=0.33 (DCM-MeOH, 8:2 v/v); LC-MS (+ve mode): m/z=317.15 [M+H]⁺; ¹H NMR (300 MHz, CDCl₃) δ 8.39 (dd, J=8.7, 1.8 Hz, 1H, ArH), 7.77 (s, 1H, ArH), 7.67 (m, 1H, ArH), 7.33 (m, 2H, 2×ArH), 3.54 (m, 2H, CH₂), 3.32 (s, 2H, CH₂), 3.22 (m, 2H, CH₂), 3.00 (s, 6H, 2×CH₃), 1.41 (s, 6H, 2×CH₃); ¹³C NMR (75.5 MHz, CDCl₃) δ 181.1, 171.1, 137.4, 130.9, 126.5, 124.7, 124.7, 119.7, 117.7, 117.5, 58.0, 46.2, 43.6, 41.4, 26.2, 21.5.

### Example 1-74: 4-(3-(2-(dimethylamino)ethyl)-5-methoxy-1H-indol-1-yl)-2,2-dimethyl-4-oxobutanoic acid HCl salt (Compound 577)

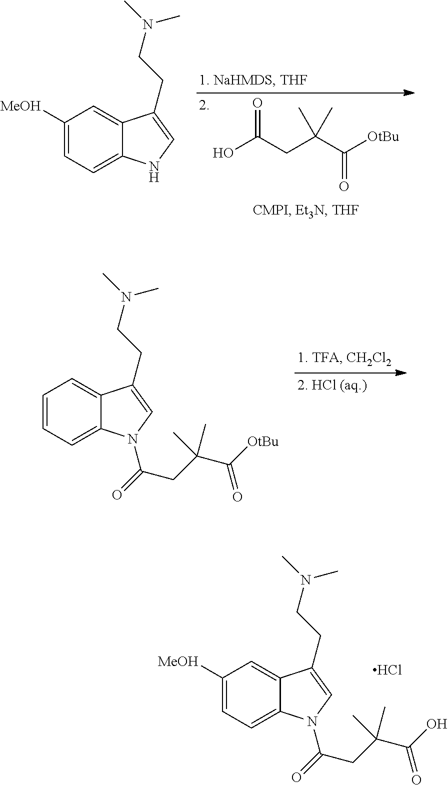

NaHMDS, 1M in THF (0.96 mL, 0.96 mmol) was added to a stirred solution of 5-OMe-DMT (200 mg, 0.92 mmol) in anhydrous THF (5 mL) at −78° C. In a separate vessel, 4-(tert-butoxy)-3,3-dimethyl-4-oxobutanoic acid (187 mg, 0.92 mmol) and 2-chloro-1-methylpyridinium iodide (220 mg, 1.01 mmol) were dissolved in anhydrous THF (5 mL). Et₃N (204 mg, 0.28 mL, 2.02 mmol) was added and the mixture was stirred at rt for 30 min, then the 5-OMe-DMT solution was added and the mixture was stirred at rt for 16 h. The solvent was removed under vacuum and the residue was purified by column chromatography on silica gel, eluting with a gradient of MeOH in DCM to afford an oil. TLC: R_{f}=0.68 (DCM-MeOH, 8:2 v/v); LC-MS (+ve mode): m/z=403.20 [M+H]⁺.

The above material was dissolved in DCM (15 mL) and TFA (2.91 g, 1.96 mL, 25.5 mmol) was added at rt. The mixture was stirred at rt for 2 h, then concentrated under vacuum and azeotroped with CHCl₃ (3×20 mL) to give an oil, which was taken up in 0.5 M HCl (2 mL) and purified by reversed-phase chromatography on silica, eluting with a gradient of acetonitrile in 0.02% hydrochloric acid to afford Compound 577 (86 mg, 24% over 2 steps) as a solid. TLC: R_{f}=0.26 (DCM-MeOH, 8:2 v/v); LC-MS (+ve mode): m/z=347.15 [M+H]⁺; ¹H NMR (300 MHz, CD₃OD) δ 8.27 (d, J=9.0 Hz, 1H, ArH), 7.66 (s, 1H, ArH), 7.12 (d, J=2.4 Hz, 1H, ArH), 6.95 (dd, J=9.0, 2.7 Hz, 1H, ArH), 3.90 (s, 3H, OCH₃), 3.51 (m, 2H, CH₂), 3.30 (s, 2H, CH₂) 3.21 (m, 2H, CH₂), 3.02 (s, 6H, 2×CH₃), 1.44 (s, 6H, 2×CH₃); ¹³C NMR (75.5 MHz, CD₃OD) δ 180.7, 170.2, 157.5, 131.4, 131.4, 124.6, 118.1, 117.0, 114.3, 102.4, 57.5, 56.1, 45.7, 43.4, 41.0, 26.1, 21.2.

### Example 2: Pharmacokinetics of Selected Compounds Following a Single Intravenous or Oral Administration in Rats

A pharmacokinetic (PK) study was performed in three male Sprague-Dawley (SD) rats following intravenous (IV) or oral (PO) administration of dimethyltryptamine (DMT), 5-methoxydimethyltryptamine (5-OMe-DMT), Compound 19 or Compound 20 at 1 mg/kg (IV) or 10 mg/kg (PO).

### In Vivo Methods.

### Rat Strain.

Sprague-Dawley rats were supplied by Charles River (Margate UK) and were specific pathogen free. Male rats weighed between 175-225 g on receipt and were allowed to acclimatize for 5-7 days.

### Animal Housing.

Rats were group housed in sterilised individual ventilated cages that exposed the animals at all times to HEPA filtered sterile air. Animals had free access to food and water (sterile) and sterile aspen chip bedding (changed at least once weekly). The room temperature was maintained at 22° C.+/−1° C., with a relative humidity of 60% and maximum background noise of 56 dB. Rats were exposed to 12-hour light/dark cycles.

### Treatment.

Each test compound and control (DMT or 5-OMe-DMT) were diluted with 10% v/v DMSO, 40% v/v PEG-400, 50% v/v water. The test compound or the control (DMT or 5-OMe-DMT) were administered in a dose volume of 2 mL/kg for intravenous administration (IV) and 5 mL/kg for oral administration (PO).

### Single IV/PO Dose Pharmacokinetics Study in Rats.

Each test compound was administered as a single IV bolus (via a lateral tail-vein) or a single oral gavage in cohorts of 3 rats per administration route. Following dose administrations, a 100 μL whole blood sample (EDTA) was collected via the tail-vein at time-points described in TABLE 8. The blood sample was centrifuged to separate plasma. Approximately 40 μL of the separated plasma was dispensed per time-point, per rat, in a 96 well plate and frozen until analysis. Bioanalysis was carried out on the separated plasma samples.

TABLE 8
Sample collection points for single IV and oral dose pharmacokinetics study.

| | | | | Dose | Blood sample collection (post | No. of |
|---|---|---|---|---|---|---|
| Group | Prodrug | Drug | Route | (mg/kg) | dose) | rats |
| 1 | Cpd 20 | DMT | IV | 1 | 5 min, 15 min, 30 min, 1 h, 2 h, | 3 |
| | | | | | 4 h, 7 h, 24 h | |
| 2 | Cpd 20 | DMT | PO | 10 | 15 min, 30 min, 45 min, 1 h, 2 h, | 3 |
| | | | | | 4 h, 7 h, 24 h | |
| 3 | Cpd 19 | 5-OMe—DMT | IV | 1 | 5 min, 15 min, 30 min, 1 h, 2 h, | 3 |
| | | | | | 4 h, 7 h, 24 h | |
| 4 | Cpd 19 | 5- | PO | 10 | 15 min, 30 min, 45 min, 1 h, 2 h, | 3 |
| | | OMe_DMT | | | 4 h, 7 h, 24 h | |

### Bioanalysis Methods.

### DMT Stock Preparation.

2.4 mL of DMSO was pipetted into an amber vial containing 2.4 mg salt-free DMT. The contents were mixed by vortex to provide a ˜1000 μg/mL standard solution in DMSO.

### 5-OMe-DMT Stock Preparation.

2.5 mL of DMSO was pipetted into amber vial containing 2.5 mg salt-free 5-OMe-DMT. The contents were mixed by vortex to provide a ˜1000 μg/mL standard solution in DM D).

### Preparation of Calibration and Quality Control Standards.

Separate calibration curve and QC standards were prepared from individual standard to minimise the chance of MRM crosstalk during analysis. The dilutions were performed as detailed in TABLE 9 and TABLE 10. Spiking volumes were 3 μL per 30 μL plasma.

TABLE 9
Preparation of 1 to 5000 ng/mL Cal and QC working solution.

| | | | |
|---|---|---|---|
| | | Starting | Starting |
| Working | Solution | Solution | Solution |
| Solution | Prepared | Conc. | Volume |
| ID | From | (μg/mL) | (μL) |

Preparation of Calibrator Working Solutions

| | | | |
|---|---|---|---|
| DMSO | — | — | — |
| WS1 | DMSO | 1000 | 50 |
| WS2 | DMSO | 1000 | 25 |
| WS3 | DMSO | 1000 | 10 |
| WS4 | WS1 | 50 | 100 |
| WS5 | WS2 | 25 | 100 |
| WS6 | WS3 | 10 | 100 |
| WS7 | WS4 | 5 | 100 |
| WS8 | WS5 | 2.5 | 100 |
| WS9 | WS6 | 1 | 100 |
| WS10 | WS7 | 0.5 | 100 |
| WS11 | WS8 | 0.25 | 100 |
| WS12 | WS9 | 0.1 | 100 |

Preparation of QC Working Solutions

| | | | |
|---|---|---|---|
| DMSO | — | — | — |
| QC-WS1 | DMSO | 1000 | 40 |
| QC-WS2 | QC-WS1 | 40 | 100 |
| QC-WS3 | QC-WS2 | 4 | 100 |
| QC-WS4 | QC-WS3 | 0.4 | 100 |

TABLE 10
Preparation of 1 to 5000 ng/mL Cal
and QC working solution (cont.).
Preparation of Calibrator Working Solutions

| | | Working | | | |
|---|---|---|---|---|---|
| Working | 50/50 | Solution | Calibrant | | |
| Solution | MeOH/H2O | Conc. | Conc. | Calibrant ID | |
| ID | Volume (μL) | (μg/mL) | (ng/mL) | (for sample list) | |
| DMSO | — | 1000 | — | — | |
| WS1 | 950 | 50 | 5000 | Cal 12 5000 | ng/mL |
| WS2 | 975 | 25 | 2500 | Cal 11 2500 | ng/mL |
| WS3 | 990 | 10 | 1000 | Cal 10 1000 | ng/mL |
| WS4 | 900 | 5 | 500 | Cal 9 500 | ng/mL |
| WS5 | 900 | 2.5 | 250 | Cal 8 250 | ng/mL |
| WS6 | 900 | 1 | 100 | Cal 7 100 | ng/mL |
| WS7 | 900 | 0.5 | 50 | Cal 6 50 | ng/mL |
| WS8 | 900 | 0.25 | 25 | Cal 5 25 | ng/mL |
| WS9 | 900 | 0.1 | 10 | Cal 4 10 | ng/mL |
| WS10 | 900 | 0.05 | 5 | Cal 3 5 | ng/mL |
| WS11 | 900 | 0.025 | 2.5 | Cal 2 2.5 | ng/mL |
| WS12 | 900 | 0.01 | 1 | Cal 1 1 | ng/mL |

Preparation of QC Working Solutions

| | | Working | | | |
|---|---|---|---|---|---|
| Working | 50/50 | Solution | QC | | |
| Solution | MeOH/H2O | Conc. | Conc. | QC ID | |
| ID | Volume (μL) | (μg/mL) | (ng/mL) | (for sample list) | |
| DMSO | — | 1000 | — | — | |
| QC-WS1 | 960 | 40 | 4000 | QC 4 4000 | ng/mL |
| QC-WS2 | 900 | 4 | 400 | QC 3 400 | ng/mL |
| QC-WS3 | 900 | 0.4 | 40 | QC 2 40 | ng/mL |
| QC-WS4 | 900 | 0.04 | 4 | QC 1 4 | ng/mL |

All samples were diluted to volume with 50:50 methanol/water (v/v) in individual 1.5 mL Eppendorf tubes and mixed by vortexing.

The control matrix was rat plasma (male Sprague Dawley, EDTA). Calibration and quality control (QC) standards were prepared by spiking control matrix with working solutions containing DMT or 5OMe-DMT.

### Dose Formulation Samples.

Dose formulation samples were diluted in two steps with 50:50 (v/v) methanol/water to an appropriate concentration, then diluted 10:90 (v/v) with control matrix to match to the calibration standard in plasma.

### Sample Extraction Procedure.

Calibration and QC standards, incurred samples, blank matrix and dose formulation samples were extracted by protein precipitation, via the addition of a bespoke acetonitrile (CH₃CN)-based Internal Standard (IS) solution, containing compounds including Metoprolol and Rosuvastatin, both of which were monitored for during analysis. Following centrifugation, a 40 μL aliquot of supernatant was diluted by the addition of 80 μL water. The prepared sample extracts were analysed by LC-MS/MS.

### Example Bioanalytical Method and Assay Procedure.

1 According to the plate layout, aliquot to wells in 0.8 mL 96-well plate (Abgene). 30 μL for Calibration, QC standards, blanks and dose formulation check.
2 Prepare Calibration and QC standards according to the assay information. Dilute dose formulation according to the assay information. Aliquot incurred samples according to the plate layout & assay information.
3 Add 90 μL of CH₃CN internal standard and vortex mix for 5 minutes at 850 rpm
4 Centrifuge at nominally 4000 rpm for 10 minutes
6 Transfer 40 μL of supernatant into a new 0.8 mL Abgene plate.
6 Add 80 μL of water to all transferred supernatant.
7 Vortex mix for 30 seconds at 1400 rpm
8 Analyse immediately by LC-MS/MS or store at +4° C. until analysis.

The analysis was performed using the following solvent system and gradient described in TABLE 11.

TABLE 11

| Instrument Name | Agilent ™ 1290 Infinity Binary HPLC Pump | | | |
|---|---|---|---|---|
| | Column Oven | | | |
| | Agilent ™ 1290 Infinity HPLC dual needle | | | |
| | injection autosampler | | | |
| Column | Kinetex ™ XB-C18, 2.6 μm, 50 × 2.1 mm | | | |
| Column Temperature | 50° C. | | | |
| Autosampler | 10° C. | | | |
| Temperature | | | | |
| Mobile Phase | Eluent A: 2.5 mmol/L ammonium formate (aq) + | | | |
| | 0.1% formic acid (v/v) | | | |
| | Eluent B: Methanol | | | |
| | Time | Flow Rate | % Mobile | % Mobile |
| | (min) | (μl/min) | Phase A | Phase B |
| Gradient Profile | 0 | 800 | 98 | 2 |
| | 0.1 | 800 | 98 | 2 |
| | 1 | 800 | 5 | 95 |
| | 1.5 | 800 | 5 | 95 |
| | 1.55 | 800 | 98 | 2 |
| | 1.8 | 800 | 98 | 2 |

| | |
|---|---|
| Flow | 0.8 mL/min |
| Stop time | 1.8 minutes |
| Injection Volume | 2 μL |

Mass spectrometer parameters for detection of DMT and 5OMe-DMT in blood plasma are provided in TABLE 12.

TABLE 12
Instrument: ABSciex 6500 QTrap using an ESI source in positive ion mode.

| | Precursor | Product | Dwell time | | | CXP | Ionisation |
|---|---|---|---|---|---|---|---|
| Compound | ion (m/z) | ion (m/z) | (ms) | DP (V) | CE (V) | (V) | Mode |
| DMT | 189.1 | 58.1 | 10.0 | 25.0 | 16.3 | 6.8 | +ve |
| DMT | 189.1 | 144.3 | 10.0 | 25.0 | 24.3 | 15.3 | +ve |
| OMe—DMT | 219.1 | 58.0 | 10.0 | 21.4 | 16.9 | 13.2 | +ve |
| OMe—DMT | 219.1 | 159.2 | 10.0 | 21.4 | 36.4 | 18.6 | +ve |

### Example 2-1: In Vivo Pharmacokinetic Analysis of DMT

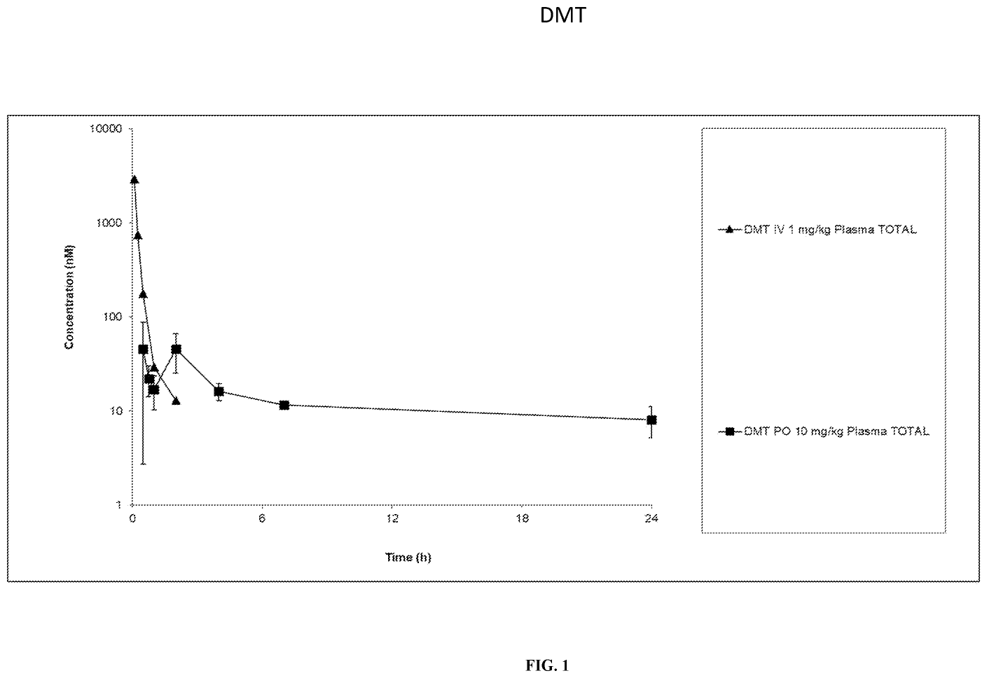

The pharmacokinetic properties of DMT after IV (1 mg/kg) and oral administration (10 mg/kg) in a rat model were assessed. The PK parameters of DMT are summarized in Table 2-1. The mean concentration-time profiles of DMT following oral dosing of DMT to Male SD rats (1 mg/kg for IV dosing, and 10 mg/kg for oral dosing) are shown in FIG. 1.

TABLE 2-1
PK Parameters of DMT

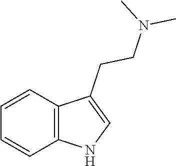

| | DMT IV | | DMT PO | | DMT IV 1 | | DMT PO 10 | |
|---|---|---|---|---|---|---|---|---|
| | 1 mg/kg | | 10 mg/kg | | mg/kg Plasma | | mg/kg Plasma | |
| | Plasma | | Plasma | | (t = 0 to 1 hr) | | (t = 0 to 1 hr) | |
| | Mean/ | | Mean/ | | Mean/ | SD | Mean/ | |
| PK Parameter | Median | SD | Median | SD | Median | | Median | SD |
| Dose (mg/kg) | 1.00 | — | 10.0 | — | 1.00 | — | 10.0 | — |
| C0/Cmax (ng/mL) | 2001 | — | 13.1 | 3.93 | 2001 | — | 8.87 | 7.63 |
| C0/Cmax (nM) | 10629 | — | 69.4 | 20.9 | 10629 | — | 47.1 | 40.5 |
| Clast (ng/mL) | 7.12 | — | 1.52 | 0.556 | 5.51 | — | 3.18 | 1.25 |
| tlast (h) | 1.50 | — | 24.0 | — | 1.00 | — | 1.02 | — |
| tmax (h) | — | — | 2.00 | — | — | — | 2.00 | — |
| t1/2 (h) | 0.146 | — | 21.1 | 6.22 | 0.140 | — | — | — |
| MRT (h) | 0.152 | — | — | — | 0.132 | — | — | — |
| Vdss (L/kg) | 0.774 | — | — | — | 0.645 | — | — | — |
| CL/CL_F | 85.8 | — | 1639 | 503 | 87.1 | — | — | — |
| (mL/min/kg) | | | | | | | | |
| AUCinf (ng.hr/mL) | 216 | — | — | — | 214 | — | — | — |
| AUCinf (nM.hr) | 1145 | — | — | — | 1136 | — | — | — |
| AUC0-t (ng.hr/mL) | 214 | — | 59.1 | 3.65 | 213 | — | 4.51 | 2.86 |
| AUC0-t (nM.hr) | 1136 | — | 314 | 19.4 | 1129 | — | 23.9 | 15.2 |
| Fraction Absorbed | — | — | — | — | — | — | — | — |
| Bioavailability (%) | — | — | — | — | — | — | — | — |
| Using AUCinf | | | | | | | | |
| Bioavailability (%) | — | — | — | — | — | — | 0.212 | 0.134 |
| Using AUC0-t | | | | | | | | |
| Number of Points | 3.00 | — | 3.33 | 0.577 | 3.50 | — | — | — |
| used for Lambda z | | | | | | | | |
| AUC % | 0.690 | — | 42.6 | 14.3 | 0.502 | — | — | — |
| Extrapolation to | | | | | | | | |
| infinity | | | | | | | | |
| AUC % Back | 66.4 | — | — | — | 67.2 | — | — | — |
| Extrapolation to | | | | | | | | |
| C0 | | | | | | | | |

### Example 2-2: In Vivo Pharmacokinetic Analysis of 5-MeO-DMT

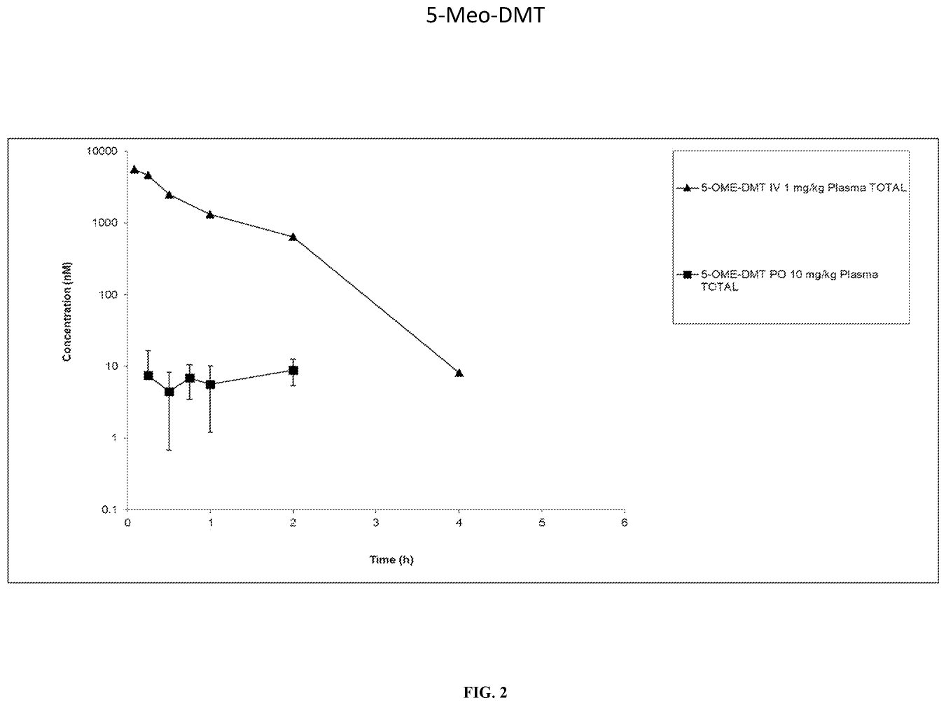

The pharmacokinetic properties of 5-MeO-DMT after IV (1 mg/kg) and oral administration (10 mg/kg) in a rat model were assessed. The PK parameters of 5-MeO-DMT are summarized in Table 2-2. The mean concentration-time profiles of DMT following oral dosing of 5-MeO-DMT to Male SD rats (1 mg/kg for IV dosing, and 10 mg/kg for oral dosing) are shown in FIG. 2.

TABLE 2-2
PK Parameters of 5-MeO-DMT

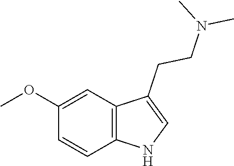

| | 5-MeO-DMT IV | | 5-MeO-DMT PO | |
|---|---|---|---|---|
| | 1 mg/kg | | 10 mg/kg | |
| | Plasma | | Plasma | |
| PK | Mean/ | | Mean/ | |
| Parameter | Median | SD | Median | SD |
| Dose (mg/kg) | 1.00 | — | 10.0 | — |
| C0/Cmax | 1889 | — | 3.03 | 1.55 |
| (ng/mL) | | | | |
| C0/Cmax | 8655 | — | 13.9 | 7.09 |
| (nM) | | | | |
| Clast (ng/mL) | 1.68 | — | 1.95 | 0.784 |
| tlast (h) | 5.50 | — | 2.00 | — |
| tmax (h) | — | — | 2.00 | — |
| t1/2 (h) | 0.510 | — | — | — |
| MRT (h) | 0.624 | — | — | — |
| Vdss (L/kg) | 0.818 | — | — | — |
| CL/CL_F | 24.6 | — | — | — |
| (mL/min/kg) | | | | |
| AUCinf | 1081 | — | — | — |
| (ng.hr/mL) | | | | |
| AUCinf | 4953 | — | — | — |
| (nM.hr) | | | | |
| AUC0-t | 1080 | — | — | — |
| (ng.hr/mL) | | | | |
| AUC0-t | 4948 | — | — | — |
| (nM.hr) | | | | |
| Number of | 6.00 | — | — | — |
| Points used for | | | | |
| Lambda z | | | | |
| AUC % | 0.204 | — | — | — |
| Extrapolation | | | | |
| to infinity | | | | |
| AUC % Back | 26.8 | — | — | — |
| Extrapolation | | | | |
| to C0 | | | | |

### Example 2-3: Pharmacokinetic Analysis of Compound 20

FIG. 3, Panel A is a chart that depicts (1) the time course of blood plasma concentrations of N,N-dimethyltryptamine (DMT) (triangular points) and Compound 20 (square points) in Sprague-Dawley rats that were intravenously (IV) administered Compound 20 at 1 mg/kg, and (2) the time course of blood plasma concentrations of N,N-dimethyltryptamine (DMT) in Sprague-Dawley rats intravenously (IV) administered 1 mg/kg DMT as a control (circle points).

FIG. 3, Panel B is a chart that depicts (1) the time course of blood plasma concentrations of N,N-dimethyltryptamine (DMT) (triangular points) and Compound 20 (square points) in Sprague-Dawley rats that were orally (PO) administered Compound 20 at 10 mg/kg, and (2) the time course of blood plasma concentrations of N,N-dimethyltryptamine (DMT) in Sprague-Dawley rats orally (PO) administered 10 mg/kg DMT as a control (circle points). TABLE 2-3 provides corresponding quantitative values for the data series represented by triangular points in FIG. 3, Panel B.

TABLE 2-3

| | |
|---|---|
| | Summary of DMT conc. determined |
| | following PO dosing of Compound 20 |
| Nominal Sampling | to male Sprague Dawley rat at 10 mg/kg |

| Timepoint (h) | Mean Concentration (nM) | SD (nM) |
|---|---|---|
| 0.25 | 782 | 223 |
| 0.50 | 1637 | 197 |
| 0.75 | 1619 | 639 |
| 1.00 | 1308 | 196 |
| 2.00 | 936 | 282 |
| 4.00 | 473 | 374 |
| 7.00 | 56.8 | 43.9 |
| 24.00 | 16.28 | 8.26 |

The pharmacokinetic properties of Compound 20 after IV or oral administration in a rat model were assessed. Compound 20: Chemical name: ethyl 3-[2-(dimethylamino)ethyl]indole-1-carboxylate; Structural class: carbamate; Mechanistic class: presumed carboxyesterases.

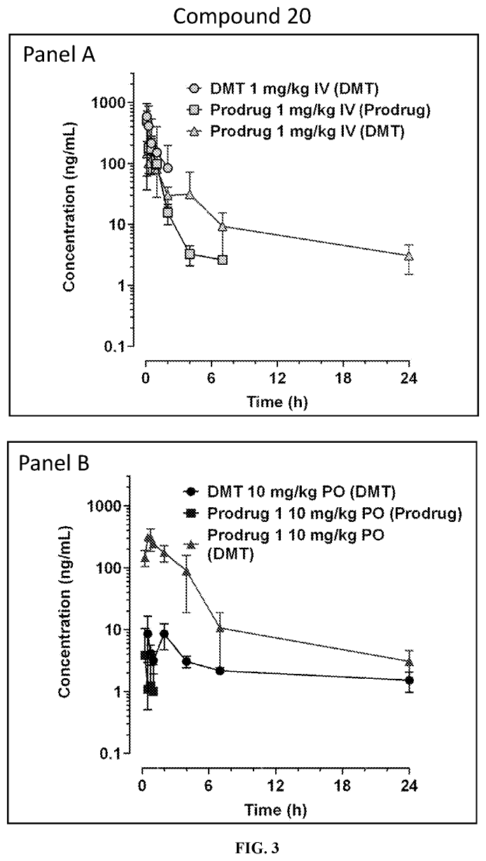

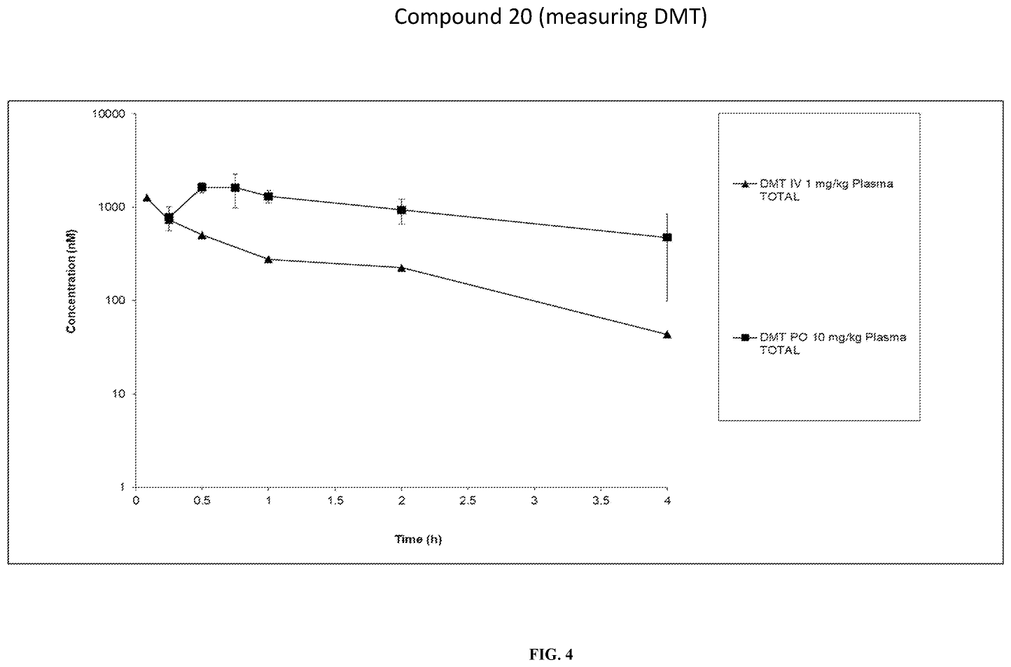

The PK parameters of Compound 20 are summarized in Table 2-3A. The mean concentration-time profiles of DMT following IV or oral dosing of Compound 20 to Male SD rats (1 mg/kg for IV dosing, 10 mg/kg for oral dosing) are shown in FIG. 4. The mean concentration-time profiles of Compound 20 following IV or oral dosing of Compound 20 to Male SD rats (1 mg/kg for IV dosing, 10 mg/kg for oral dosing) are shown in FIG. 5.

TABLE 2-3A
PK parameters of Compound 20 after IV
or Oral Administration of Compound 20

| | Compound 20 IV | | Compound 20 PO | |
|---|---|---|---|---|
| | 1 mg/kg Plasma | | 10 mg/kg Plasma | |
| PK Parameter | Mean/Median | SD | Mean/Median | SD |
| Dose (mg/kg) | 1.00 | — | 10.0 | — |
| C0/Cmax (ng/mL) | 936 | 1100 | 11.5 | — |
| C0/Cmax (nM) | 3594 | 4228 | 44.3 | — |
| Clast (ng/mL) | 3.26 | 1.20 | 3.01 | — |
| tlast (h) | 4.00 | | 1.00 | — |
| tmax (h) | — | | 0.250 | — |
| t½ (h) | 0.614 | 0.0440 | — | — |
| MRT (h) | 0.810 | 0.216 | — | — |
| Vdss (L/kg) | 3.53 | 2.05 | — | — |
| CL/CL_F | 67.9 | 27.2 | — | — |
| (mL/min/kg) | | | | |
| AUCinf (ng · hr/mL) | 282 | 138 | — | — |
| AUCinf (nM · hr) | 1084 | 530 | — | — |
| AUC0-t (ng · hr/mL) | 279 | 137 | 4.78 | — |
| AUC0-t (nM · hr) | 1072 | 526 | 18.4 | — |
| AUC % Extrapolation | 1.073 | 0.2440 | — | — |
| to infinity | | | | |
| AUC % Back | 17.5 | 11.8 | — | — |
| Extrapolation to C0 | | | | |

### Example 2-4: In Vivo Pharmacokinetic Analysis of Compound 19

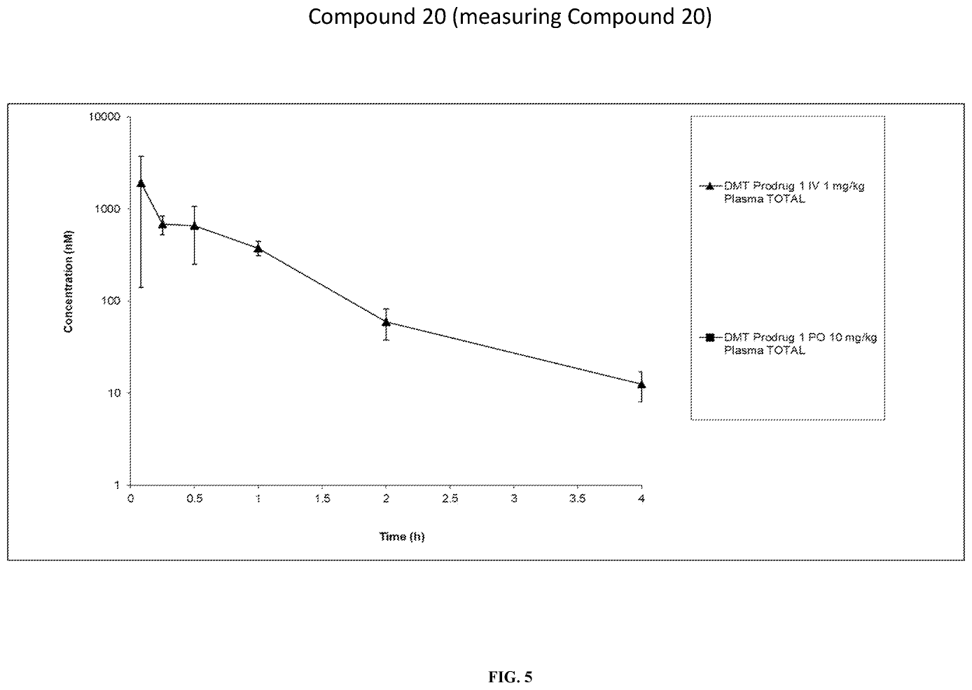

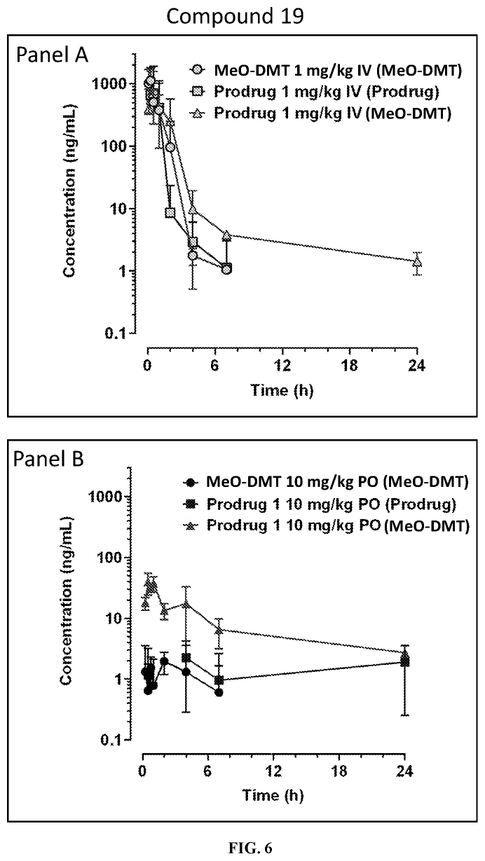

FIG. 6, Panel A is a chart that depicts (1) the time course of blood plasma concentrations of 5-methoxy-N,N-dimethyltryptamine (5-MeO-DMT) (triangular points) and Compound 19 (square points) in Sprague-Dawley rats that were intravenously (IV) administered Compound 19 at 1 mg/kg, and (2) the time course of blood plasma concentrations of 5-methoxy-N,N-dimethyltryptamine (5-MeO-DMT) in Sprague-Dawley rats intravenously (IV) administered 1 mg/kg 5-MeO-DMT as a control (circle points).

FIG. 6, Panel B is a chart that depicts (1) the time course of blood plasma concentrations of 5-methoxy-N,N-dimethyltryptamine (5-MeO-DMT) (triangular points) and Compound 19 (square points) in Sprague-Dawley rats that were orally (P0) administered Compound 19 at 10 mg/kg, and (2) the time course of blood plasma concentrations of 5-methoxy-N,N-dimethyltryptamine (5-MeO-DMT) in Sprague-Dawley rats orally (P0) administered 10 mg/kg 5-MeO-DMT as a control (circle points). TABLE 2-4 provides corresponding quantitative values for the data series represented by triangular points in FIG. 6, Panel B.

TABLE 2-4

| | Summary of 5-OMe—DMT conc. determined | |
|---|---|---|
| | following PO dosing of Compound 19 | |
| Nominal Sampling | to male Sprague Dawley rat at 10 mg/kg | |
| Timepoint (h) | Mean Concentration (nM) | SD (nM) |
| 0.25 | 81.9 | 19.7 |
| 0.50 | 181 | 69.5 |
| 0.75 | 146 | 23.9 |
| 1.00 | 173 | 48.4 |
| 2.00 | 61.5 | 18.2 |
| 4.00 | 80.1 | 70.3 |
| 7.00 | 29.9 | 15.4 |
| 24.0 | 12.5 | 4.12 |

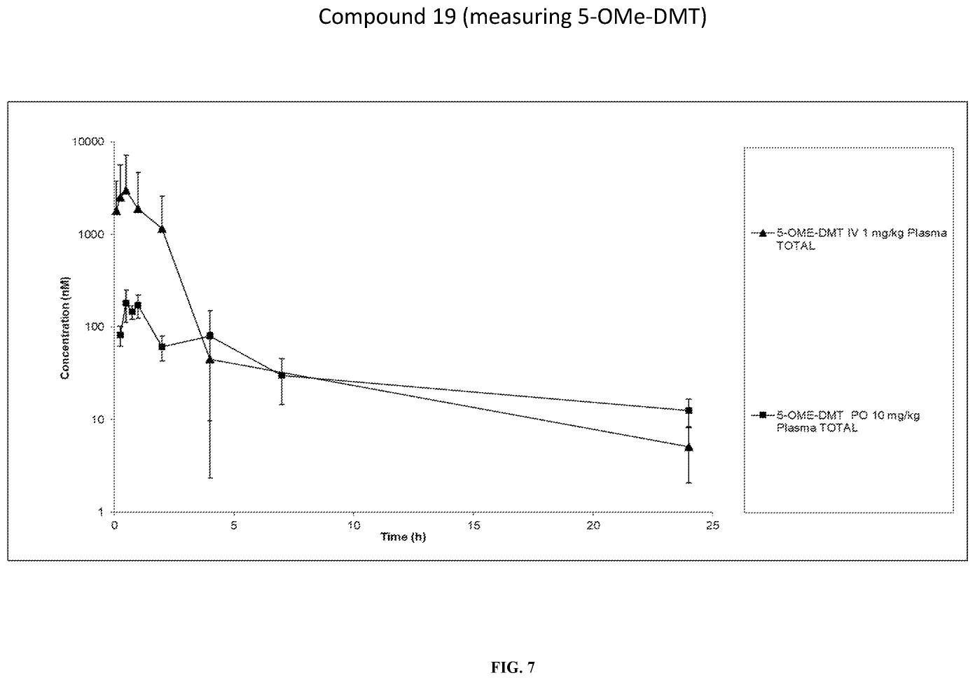

The pharmacokinetic properties of Compound 19 after IV or oral administration in a rat model were assessed. Compound 19: Chemical name: ethyl 3-[2-(dimethylamino)ethyl]-5-methoxy-indole-1-carboxylate; Structural class: carbamate; Mechanistic class: presumed carboxyesterases. 0005681 The PK parameters of Compound 19 are summarized in Table 24A. The mean concentration-time profiles of 5-methoxy-N,N-dimethyltryptamine (5-MeO-DMT) following IV or oral dosing of Compound 19 to Male SD rats (1 mg/kg for IV dosing, 10 mg/kg for oral dosing) are shown in FIG. 7.

TABLE 2-4A
PK Parameters of Compound 19 after IV
or Oral Administration of Compound 19

| | Compound 19 IV | | Compound 19 PO | |
|---|---|---|---|---|
| | 1 mg/kg Plasma | | 1 mg/kg Plasma | |
| PK Parameter | Mean/Median | SD | Mean/Median | SD |
| Dose (mg/kg) | 1.00 | — | 10.0 | — |
| C0/Cmax (ng/mL) | 53726 | — | 2.37 | 2.05 |
| C0/Cmax (nM) | 185009 | — | 8.15 | 7.06 |
| Clast (ng/mL) | 2.95 | — | 1.90 | 1.64 |
| tlast (h) | 5.50 | — | 24.0 | — |
| tmax (h) | — | — | — | — |
| t½ (h) | — | — | — | — |
| AUC0-t (ng · hr/mL) | 4172 | — | 68.8 | — |
| AUC0-t (nM · hr) | 14367 | — | 237 | — |
| Number of Points | 3.00 | — | — | — |
| used for Lambda z | | | | |
| AUC % Extrapolation | 0.104 | — | — | — |
| to infinity | | | | |
| AUC % Back | 59.7 | — | — | — |
| Extrapolation to C0 | | | | |

### Example 2-5. Diisopropylphosphonate DMT Prodrug

Chemical name: 2-(1-diisopropoxyphosphorylindol-3-yl)-N,N-dimethyl-ethanamine
Structural class: phosphonate
Mechanistic class: presumed carboxyesterases+presumed phosphatases

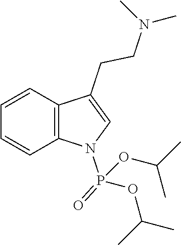 [SMILES: CC(C)OP(=O)(OC(C)C)n1cc(CCN(C)C)c2ccccc21]

FIG. 8 shows Mean Total Concentrations of DMT following P0 administration of DMT Prodrug to male Sprague Dawley rat at 10 mg/kg.

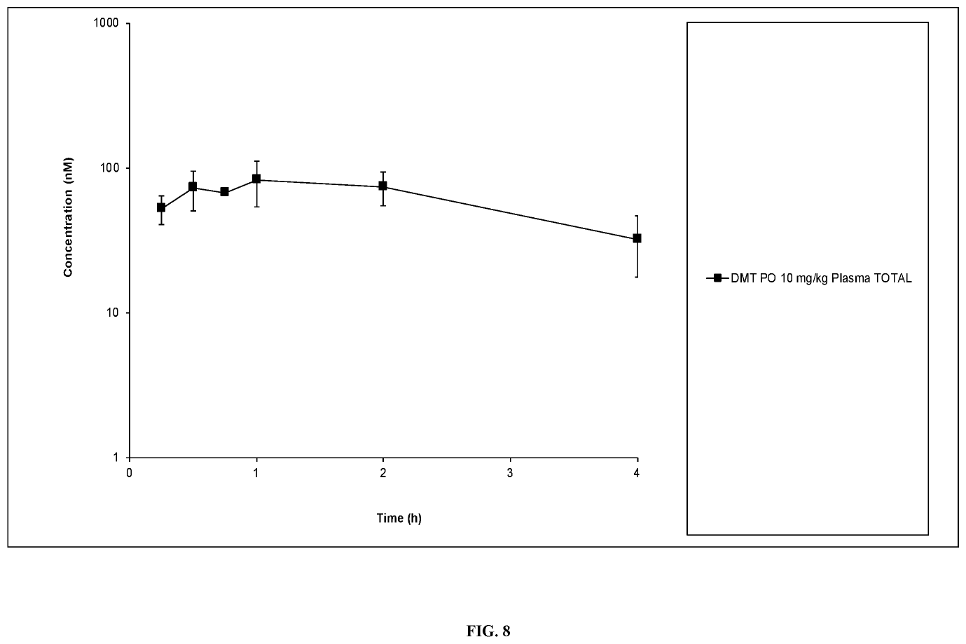

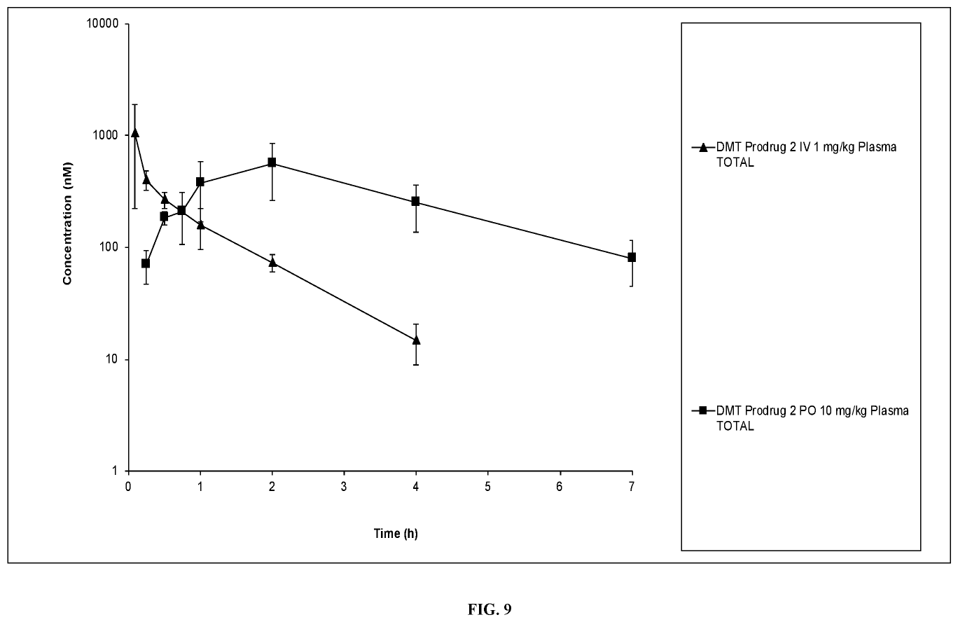

FIG. 9 shows Mean Total Concentrations of DMT Prodrug following IV, PO administration to male Sprague Dawley rat at 1.10 mg/kg.

TABLE 2-5
DMT Prodrug PK parameters

| | | | | | DMT Prodrug PO | |
|---|---|---|---|---|---|---|
| | DMT Prodrug | | DMT Prodrug PO | | 10 mg/kg Plasma | |
| | IV 1 mg/kg Plasma | | 10 mg/kg Plasma | | [Tlast = 4 h] | |
| PK | Mean/ | | Mean/ | | Mean/ | |
| Parameter | Median | SD | Median | SD | Median | SD |
| Dose (mg/kg) | 1.00 | — | 10.0 | — | 10.0 | — |
| C0/Cmax | 2239 | 3209 | 195 | 103 | 195 | 103 |
| (ng/mL) | | | | | | |
| C0/Cmax (nM) | 6355 | 9107 | 554 | 293 | 554 | 293 |
| Clast (ng/mL) | 5.22 | 2.05 | 28.2 | 12.5 | 87.8 | 39.2 |
| tlast (h) | 4.00 | — | 7.00 | — | 4.00 | — |
| tmax (h) | — | — | 2.00 | — | 2.00 | — |
| t½ (h) | 0.865 | 0.156 | — | — | — | — |
| MRT (h) | 0.869 | 0.434 | — | — | — | — |
| Vdss (L/kg) | 4.21 | 3.05 | — | — | — | — |
| CL/CL_F | 70.2 | 35.9 | — | — | — | — |
| (mL/min/kg) | | | | | | |
| AUCinf | 311 | 217 | — | — | — | — |
| (ng · hr/mL) | | | | | | |
| AUCinf (nM · hr) | 881 | 617 | — | — | — | — |
| AUC0-t | 304 | 220 | 644 | 290 | 489 | 235 |
| (ng · hr/mL) | | | | | | |
| AUC0-t (nM · hr) | 862 | 625 | 1828 | 822 | 1389 | 666 |
| Bioavailability | — | — | — | — | 16.1 | 7.72 |
| (%) Using | | | | | | |
| AUC0-t | | | | | | |
| Number of | 4 | — | — | — | — | — |
| Points used for | | | | | | |
| Lambda z | | | | | | |
| AUC % | 3.21 | 2.39 | — | — | — | — |
| Extrapolation to | | | | | | |
| infinity | | | | | | |
| AUC % Back | 36.7 | 25.8 | — | — | — | — |
| Extrapolation to | | | | | | |
| C0 | | | | | | |

### Example 2-6. Diisopropylphosphonate 5-MeO-DMT Prodrug

Chemical name: 2-(1-diisopropoxyphosphoryl-5-methoxy-indol-3-yl)-N,N-dimethyl-ethanamine
Structural class: phosphonate
Mechanistic class: presumed carboxyesterases+presumed phosphatases

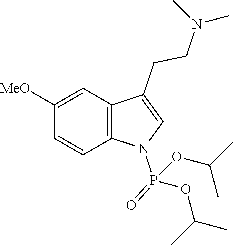 [SMILES: COc1ccc2c(c1)c(CCN(C)C)cn2P(=O)(OC(C)C)OC(C)C]

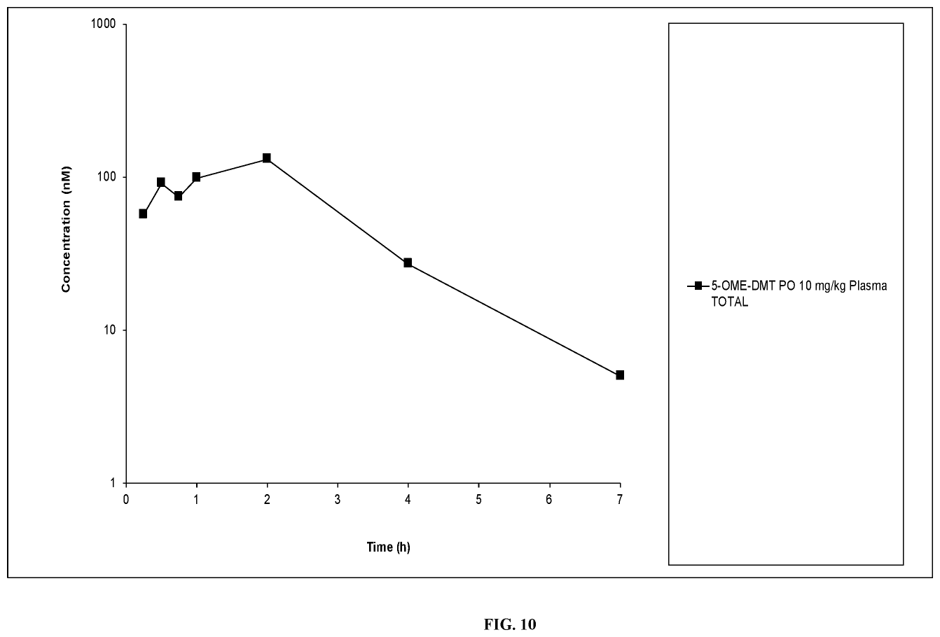

FIG. 10. Mean Total concentrations of 5-MeO-DMT following P0 administration of 5-MeO-DMT Pro-drug to male Sprague Dawley rat at 10 mg/kg.

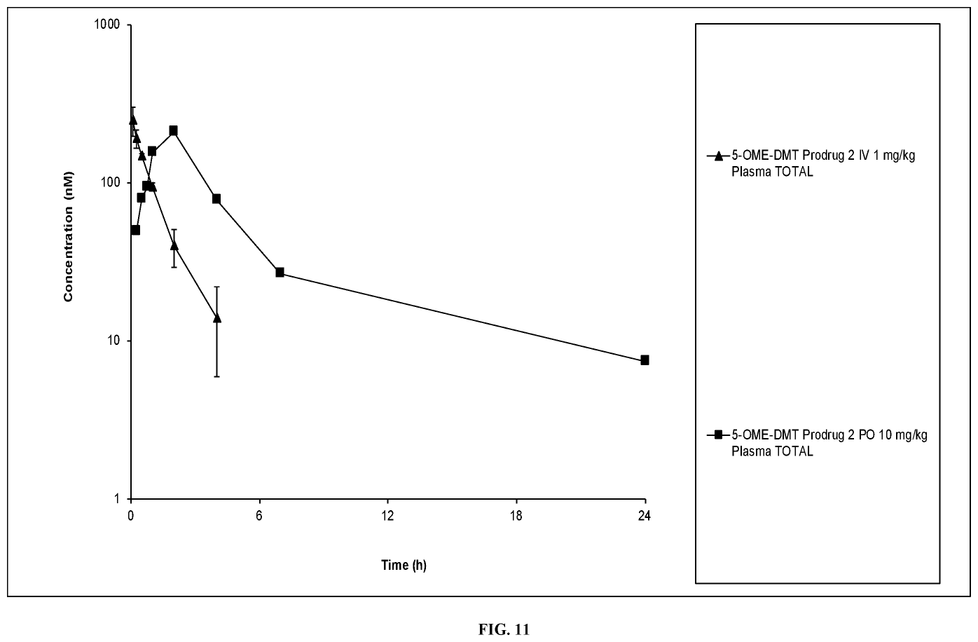

FIG. 11. Mean Total concentrations of 5-MeO-DMT Prodrug following IV, PO administration to male Sprague Dawley rat at 1.10 mg/kg.

TABLE 2-6
5-MeO—DMT Prodrug PK Parameters

| | 5-MeO—DMT | | 5-MeO—DMT |
|---|---|---|---|
| | Prodrug IV | | Prodrug PO |
| | 1 mg/kg Plasma | | 10 mg/kg Plasma |
| PK Parameter | Mean/Median | SD | Mean/Median |
| Dose (mg/kg) | 1.00 | — | 10.0 |
| C0/Cmax (ng/mL) | 112 | 37.2 | 80.4 |
| C0/Cmax (nM) | 294 | 97.2 | 210 |
| Clast (ng/mL) | 5.32 | 3.04 | 2.84 |
| tlast (h) | 4.00 | — | 24.1 |
| tmax (h) | — | — | 2.00 |
| t½ (h) | 1.01 | 0.229 | 7.00 |
| MRT (h) | 1.39 | 0.358 | — |
| Vdss (L/kg) | 12.2 | 1.32 | — |
| CL/CL_F | 150 | 22.8 | 450 |
| (mL/min/kg) | | | |
| AUCinf (ng · hr/mL) | 113 | 18.1 | 381 |
| AUCinf (nM · hr) | 295 | 47.3 | 995 |
| AUC0-t (ng · hr/mL) | 104 | 11.6 | 352 |
| AUC0-t (nM · hr) | 273 | 30.3 | 921 |
| Bioavailability (%) | — | — | 33.8 |
| Using AUCinf | | | |
| Number of Points | 5 | — | 3 |
| used for Lambda z | | | |
| AUC % Extrapolation | 7.01 | 4.51 | 7.77 |
| to infinity | | | |
| AUC % Back | 7.68 | 2.16 | — |
| Extrapolation to C0 | | | |

### Example 2-7. Isopropyl Carbamate DMT Prodrug

| Dosed Test Article: | | DMT CP-2 |
|---|---|---|
| Dose Route: | | Oral |
| Nominal Dose Concentration: | | 10 mg/Kg |
| Analytes: | Pro-Drug | DMT CP-2 |
| | Metabolite | DMT |

Chemical name: isopropyl 3-[2-(dimethylamino)ethyl]indole-1-carboxylate
Structural class: carbamate
Mechanistic class: presumed carboxyesterases

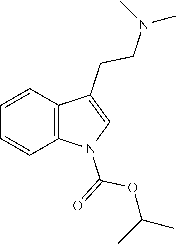 [SMILES: CC(C)OC(=O)n1cc(CCN(C)C)c2ccccc21]

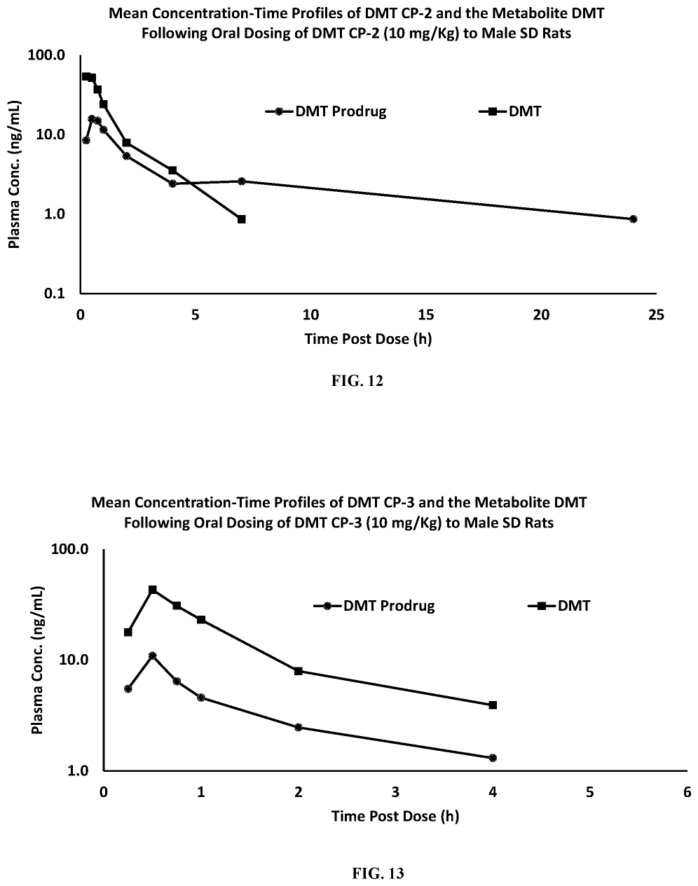

FIG. 12. Mean Concentration-Time Profiles of DMT CP-2 and Metabolite DMT Following Oral Dosing of DMT CP-2 (10 mg/Kg) to Male SD Rats

TABLE 2-7
DMT Prodrug and DMT PK Parameters
Mean Pharmacokinetic Parameters

| PK Parameter | DMT CP-2 | DMT |
|---|---|---|
| Cmax (ng/mL) | 17.4 | 58.8 |
| Tmax (h) | 0.500 | 0.500 |
| MRT (h) | 2.96 | 1.32 |
| Tlast (h) | 4.00 | 4.00 |
| AUC0-last (h * ng/mL) | 39.4 | 65.5 |
| AUC0-24 (h * ng/mL) | 60.9 | — |
| AUC0-inf (h * ng/mL) | 45.5 | 69.0 |
| T½ (h) | 4.04 | 1.21 |

* Median calculated for Tmax and Tlast.

### Example 2-8. tert-butyl Carbamate DMT Prodrug

| Dosed Test Article: | | DMT CP-3 |
|---|---|---|
| Dose Route: | | Oral |
| Nominal Dose Concentration: | | 10 mg/Kg |
| Analytes: | Pro-Drug | DMT CP-3 |
| | Metabolite | DMT |

Chemical name: tert-butyl 3-[2-(dimethylamino)ethyl]indole-1-carboxylate
Structural class: carbamate
Mechanistic class: presumed carboxyesterases

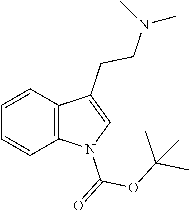 [SMILES: CN(C)CCc1cn(C(=O)OC(C)(C)C)c2ccccc12]

FIG. 13. Mean Concentration-Time Profiles of DMT CP-3 and the Metabolite DMT Following Oral Dosing of DMT CP-3 (10 mg/Kg) to Male SD Rats

TABLE 2-8
DMT Prodrug and DMT PK Parameters
Mean Pharmacokinetic Parameters

| PK Parameter | DMT Prodrug | DMT |
|---|---|---|
| Cmax (ng/mL) | 11.1 | 44.1 |
| Tmax (h) | 0.500 | 0.500 |
| MRT (h) | 1.39 | 1.28 |
| Tlast (h) | 4.00 | 4.00 |
| AUC0-last (h * ng/mL) | 13.8 | 55.0 |
| AUC0-24 (h * ng/mL) | — | — |
| AUC0-inf (h * ng/mL) | 17.5 | 64.1 |
| T½ (h) | 1.87 | 1.35 |

* Median calculated for Tmax and Tlast.

### Example 2-9. Propyl Carbamate DMT Prodrug

| Dosed Test Article: | | DMT CP-4 |
|---|---|---|
| Dose Route: | | Oral |
| Nominal Dose Concentration: | | 10 mg/Kg |
| Analytes: | Pro-Drug | DMT CP-4 |
| | Metabolite | DMT |

Chemical name: propyl 3-[2-(dimethylamino)ethyl]indole-1-carboxylate
Structural class: carbamate
Mechanistic class: presumed carboxyesterases

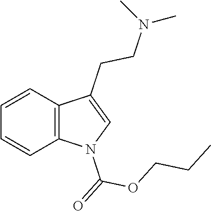 [SMILES: CCCOC(=O)n1cc(CCN(C)C)c2ccccc21]

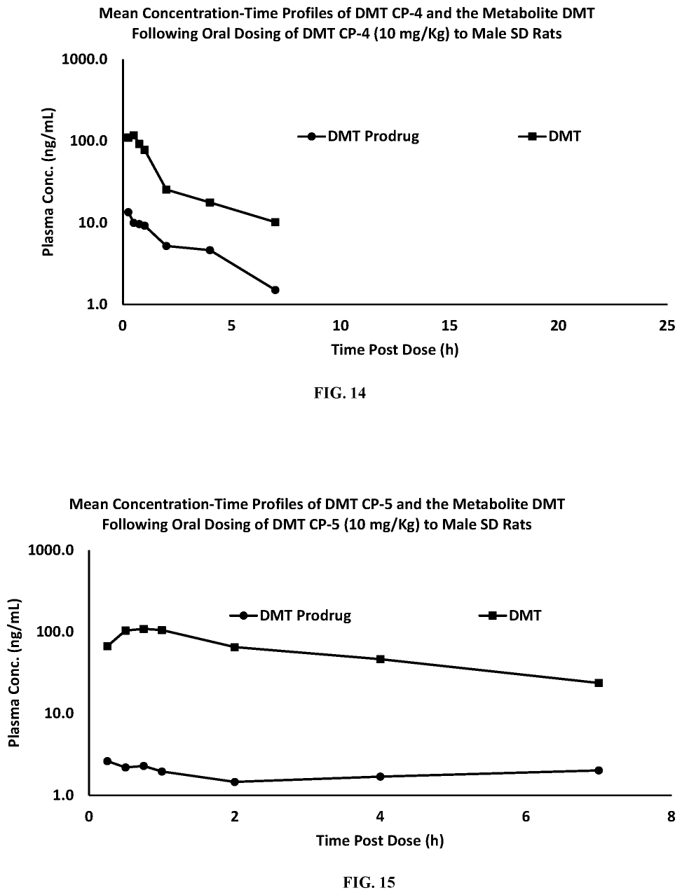

FIG. 14. Mean Concentration-Time Profiles of DMT CP-4 and the Metabolite DMT Following Oral Dosing of DMT CP-4 (10 mg/Kg) to Male SD Rats

TABLE 2-9
DMT Prodrug and DMT PK Parameters
Mean Pharmacokinetic Parameters

| PK Parameter | DMT Prodrug | DMT |
|---|---|---|
| Cmax (ng/mL) | 14.3 | 128 |
| Tmax (h) | 0.375 | 0.500 |
| MRT (h) | 1.46 | 1.95 |
| Tlast (h) | 4.50 | 7.00 |
| AUC0-last (h * ng/mL) | 28.2 | 227.0 |
| AUC0-24 (h * ng/mL) | — | — |
| AUC0-inf (h * ng/mL) | 30.9 | 306.0 |
| T½ (h) | 1.35 | 4.6 |

*Median calculated for Tmax and Tlast.

### Example 2-10. Isobutyl Carbamate DMT Prodrug

| Dosed Test Article: | | DMT CP-5 |
|---|---|---|
| Dose Route: | | Oral |
| Nominal Dose Concentration: | | 10 mg/Kg |
| Analytes: | Pro-Drug | DMT CP-5 |
| | Metabolite | DMT |

Chemical name: isobutyl 3-[2-(dimethylamino)ethyl]indole-1-carboxylate
Structural class: carbamate
Mechanistic class: presumed carboxyesterases

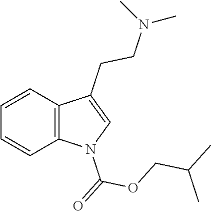 [SMILES: CC(C)COC(=O)n1cc(CCN(C)C)c2ccccc21]

FIG. 15. Mean Concentration-Time Profiles of DMT CP-5 and the Metabolite DMT Following Oral Dosing of DMT CP-5 (10 mg/Kg) to Male SD Rats

TABLE 2-10
DMT Prodrug and DMT PK Parameters
Mean Pharmacokinetic Parameters

| PK Parameter | DMT Prodrug | DMT |
|---|---|---|
| Cmax (ng/mL) | 2.92 | 120 |
| Tmax (h) | 1.000 | 0.500 |
| MRT (h) | 2.09 | 2.63 |
| Tlast (h) | 4.00 | 7.00 |
| AUC0-last (h * ng/mL) | 8.63 | 386.0 |
| AUC0-24 (h * ng/mL) | — | — |
| AUC0-inf (h * ng/mL) | 71.5 | 443.0 |
| T½ (h) | 19.10 | 1.84 |

* Median calculated for Tmax and Tlast.

### Example 2-11. Methyl amide DMT Prodrug

| Dosed Test Article: | | DMT AP-1 |
|---|---|---|
| Dose Route: | | Oral |
| Nominal Dose Concentration: | | 10 mg/Kg |
| Analytes: | Pro-Drug | DMT AP-1 |
| | Metabolite | DMT |

Chemical name: 1-[3-[2-(dimethylamino)ethyl]indol-1-yl]ethenone
Structural class: amide
Mechanistic class: presumed amidases

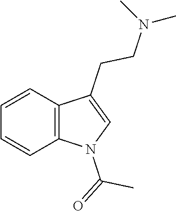 [SMILES: CC(=O)n1cc(CCN(C)C)c2ccccc21]

FIG. 16. Mean Concentration-Time Profiles of DMT AP-1 and the Metabolite DMT Following Oral Dosing of DMT AP-1 (10 mg/Kg) to Male SD Rats

TABLE 2-11
DMT Prodrug and DMT PK Parameters
Mean Pharmacokinetic Parameters

| PK Parameter | DMT Prodrug | DMT |
|---|---|---|
| Cmax (ng/mL) | 3.53 | 2.42 |
| Tmax (h) | 0.500 | 0.500 |
| MRT (h) | 0.58 | 0.695 |
| Tlast (h) | 1.00 | 1.00 |
| AUC0-last (h * ng/mL) | 2.16 | 1.9 |
| AUC0-24 (h * ng/mL) | — | — |
| AUC0-inf (h * ng/mL) | — | 3.7 |
| T½ (h) | — | 1.13 |

* Median calculated for Tmax and Tlast.

### Example 2-12. Isopropyl Carbamate 5-MeO-DMT Prodrug

| Dosed Test Article: | | 5-MeO—DMT CP-2 |
|---|---|---|
| Dose Route: | | Oral |
| Nominal Dose Concentration: | | 10 mg/Kg |
| Analytes: | Pro-Drug | 5-MeO—DMT CP-2 |
| | Metabolite | 5-MeO—DMT |

Chemical name: isopropyl 3-[2-(dimethylamino)ethyl]-5-methoxy-indole-1-carboxylate
Structural class: carbamate
Mechanistic class: presumed carboxyesterases

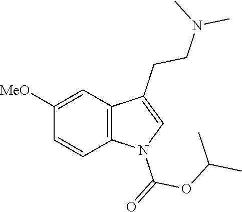 [SMILES: COc1ccc2c(c1)c(CCN(C)C)cn2C(=O)OC(C)C]

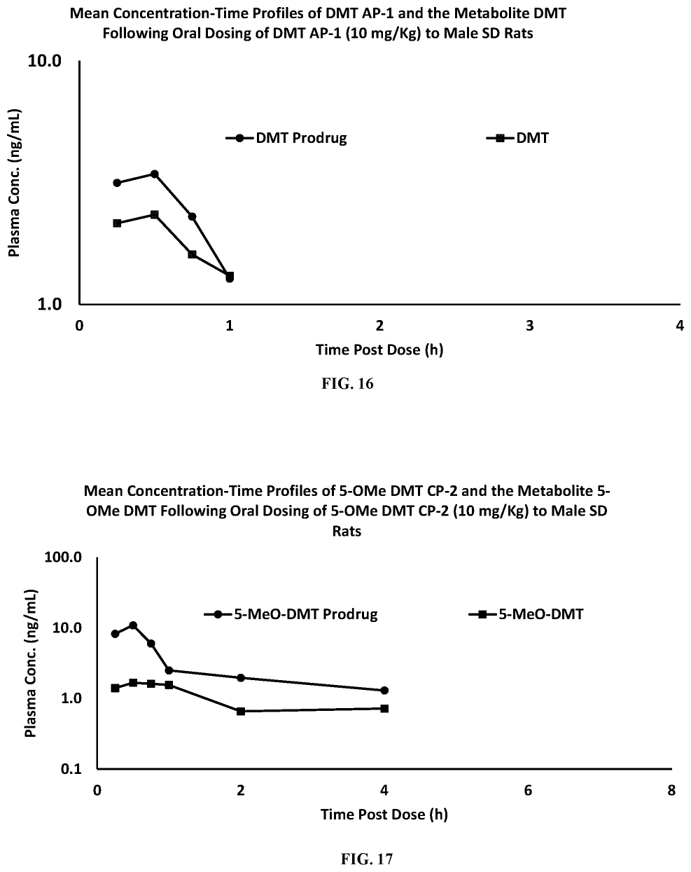

FIG. 17. Mean Concentration-Time Profiles of 5-MeO-DMT CP-2 and the Metabolite 5-MeO DMT Following Oral Dosing of 5-MeO-DMT CP-2 (10 mg/Kg) to Male SD Rats

TABLE 2-12
5-MeO—DMT Prodrug and 5-MeO—DMT PK Parameters
Mean Pharmacokinetic Parameters

| PK Parameter | 5-MeO—DMT Prodrug | 5-MeO—DMT |
|---|---|---|
| Cmax (ng/mL) | 5.7 | 1.81 |
| Tmax (h) | 0.750 | 0.500 |
| MRT (h) | 1.13 | 1.27 |
| Tlast (h) | 2.50 | 2.00 |
| AUC0-last (h * ng/mL) | 6.8 | 3.2 |
| AUC0-24 (h * ng/mL) | — | — |
| AUC0-inf (h * ng/mL) | 16.7 | 6.4 |
| T½ (h) | 1.80 | 2.35 |

* Median calculated for Tmax and Tlast.

### Example 2-13. tert-butyl Carbamate 5-MeO-DMT Prodrug

| Dosed Test Article: | | 5-MeO—DMT CP-3 |
|---|---|---|
| Dose Route: | | Oral |
| Nominal Dose Concentration: | | 10 mg/Kg |
| Analytes: | Pro-Drug | 5-MeO DMT CP-3 |
| | Metabolite | 5-MeO—DMT |

Chemical name: tert-butyl 3-[2-(dimethylamino)ethyl]-5-methoxy-indole-1-carboxylate
Structural class: carbamate
Mechanistic class: presumed carboxyesterases

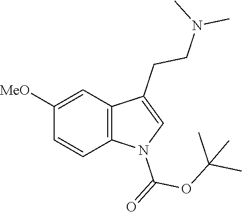 [SMILES: COc1ccc2c(c1)c(CCN(C)C)cn2C(=O)OC(C)(C)C]

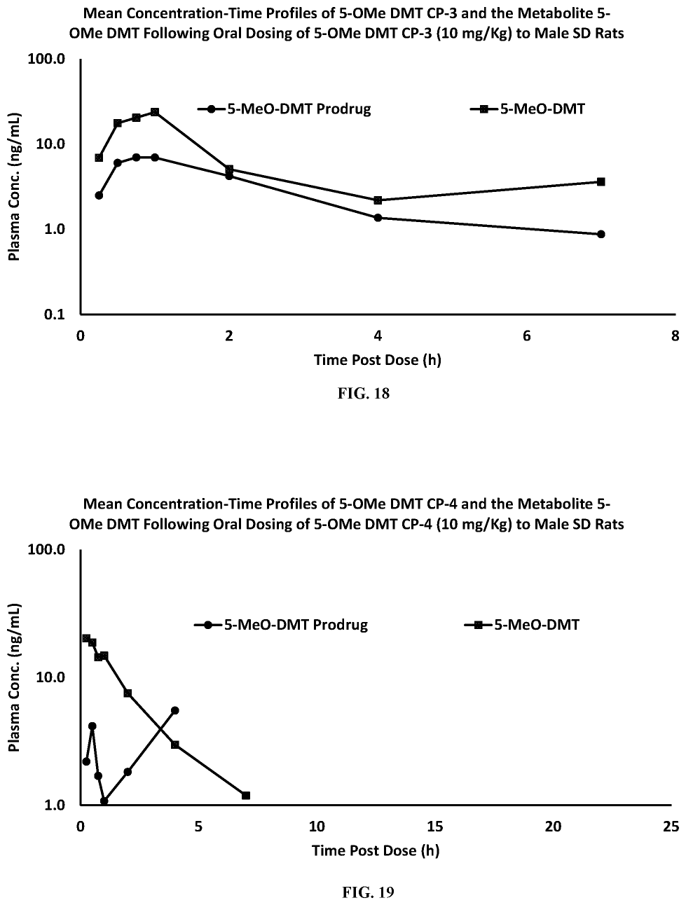

FIG. 18. Mean Concentration-Time Profiles of 5-MeO-DMT CP-3 and the Metabolite 5-MeO DMT Following Oral Dosing of 5-MeO-DMT CP-3 (10 mg/Kg) to Male SD Rats.

TABLE 2-13
5-MeO—DMT Prodrug and 5-MeO—DMT PK Parameters
Mean Pharmacokinetic Parameters

| PK Parameter | 5-MeO—DMT Prodrug | 5-MeO—DMT |
|---|---|---|
| Cmax (ng/mL) | 7.51 | 24.1 |
| Tmax (h) | 0.750 | 1.000 |
| MRT (h) | 1.84 | 1.91 |
| Tlast (h) | 4.00 | 4.00 |
| AUC0-last (h * ng/mL) | 17.3 | 38.3 |
| AUC0-24 (h * ng/mL) | — | — |
| AUC0-inf (h * ng/mL) | 25.0 | NC |
| T½ (h) | 1.58 | NC |

* Median calculated for Tmax and Tlast.

### Example 2-14. Propyl Carbamate 5-MeO-DMT Prodrug

Chemical name: propyl 3-[2-(dimethylamino)ethyl]-5-methoxy-indole-1-carboxylate
Structural class: carbamate
Mechanistic class: presumed carboxyesterase

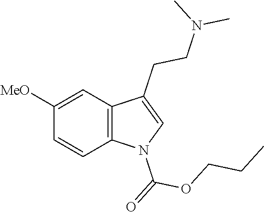 [SMILES: CCCOC(=O)n1cc(CCN(C)C)c2cc(OC)ccc21]

FIG. 19. Mean Concentration-Time Profiles of 5-MeO-DMT CP-4 and the Metabolite 5-MeO DMT Following Oral Dosing of 5-MeO-DMT CP-4 (10 mg/Kg) to Male SD Rats

TABLE 2-14
5-MeO—DMT Prodrug and 5-MeO—DMT PK Parameters
Mean Pharmacokinetic Parameters

| PK Parameter | 5-MeO—DMT Prodrug | 5-MeO—DMT |
|---|---|---|
| Cmax (ng/mL) | 4.85 | 24 |
| Tmax (h) | 2.250 | 0.500 |
| MRT (h) | 2.05 | 1.87 |
| Tlast (h) | 3.00 | 7.00 |
| AUC0-last (h * ng/mL) | 6.45 | 43.1 |
| AUC0-24 (h * ng/mL) | — | — |
| AUC0-inf (h * ng/mL) | NC | 47.2 |
| T½ (h) | NC | 2.01 |

* Median calculated for Tmax and Tlast.

### Example 2-15. Isobutyl Carbamate 5-MeO-DMT Prodrug

Chemical name: Isobutyl 3-[2-(dimethylamino)ethyl]-6-methoxy-indole-1-carboxylate
Structural class: carbamate
Mechanistic class: presumed carboxyesterases

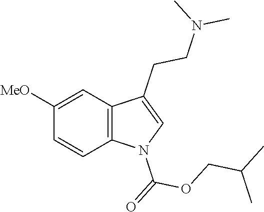 [SMILES: COc1ccc2c(c1)c(CCN(C)C)cn2C(=O)OCC(C)C]

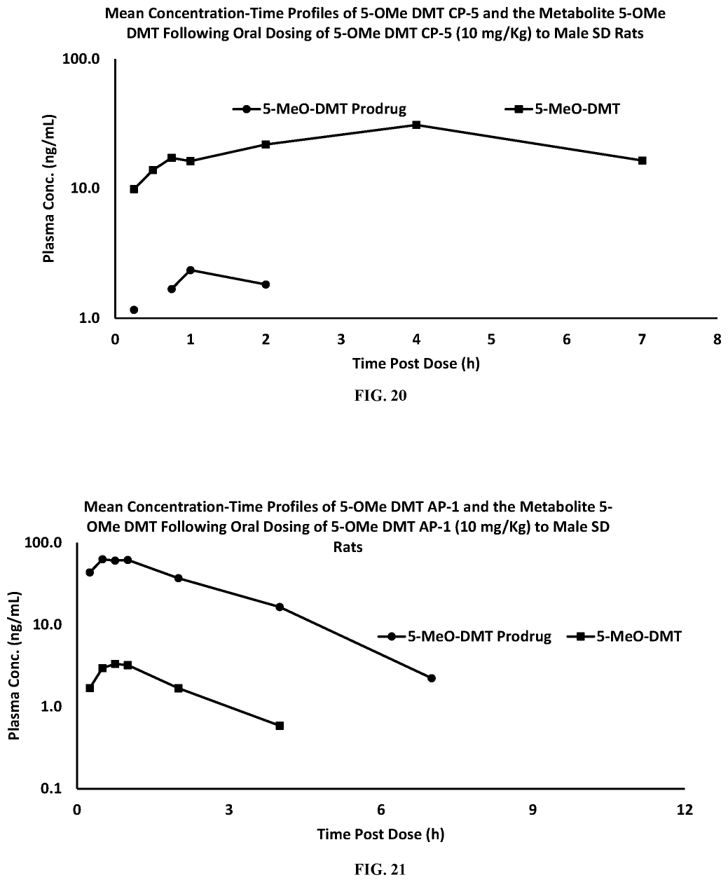

FIG. 20. Mean Concentration-Time Profiles of 5-MeO-DMT CP-5 and the Metabolite 5-MeO-DMT Following Oral Dosing of 5-MeO-DMT CP-5 (10 mg/Kg) to Male SD Rats

TABLE 2-15
5-MeO—DMT Prodrug and 5-MeO—DMT PK Parameters
Mean Pharmacokinetic Parameters

| PK Parameter | 5-MeO—DMT Prodrug | 5-MeO—DMT |
|---|---|---|
| Cmax (ng/mL) | 2.09 | 35.1 |
| Tmax (h) | 1.500 | 4.000 |
| MRT (h) | 1.07 | 3.81 |
| Tlast (h) | 1.50 | 7.00 |
| AUC0-last (h * ng/mL) | 2.11 | 155.0 |
| AUC0-24 (h * ng/mL) | — | — |
| AUC0-inf (h * ng/mL) | NC | NC |
| T½ (h) | NC | NC |

* Median calculated for Tmax and Tlast.

### Example 2-16. Methyl amide 5-MeO-DMT Prodrug

Chemical name: 1-[3-[2-(dimethylamino)ethyl]-5-methoxy-indol-1-yl]ethanone
Structural class: amide
Mechanistic class: presumed amidases

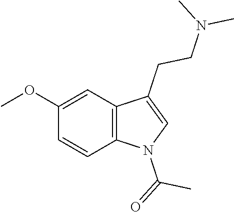

FIG. 21. Mean Concentration-Time Profiles of 5-MeO-DMT AP-1 and the Metabolite 5-MeO-DMT Following Oral Dosing of 5-MeO-DMT AP-1 (10 mg/Kg) to Male SD Rats

TABLE 2-16
5-MeO—DMT Prodrug and 5-MeO—DMT PK Parameters
Mean Pharmacokinetic Parameters

| PK Parameter | 5-MeO—DMT Prodrug | 5-MeO—DMT |
|---|---|---|
| Cmax (ng/mL) | 69 | 3.5 |
| Tmax (h) | 0.500 | 0.750 |
| MRT (h) | 2.06 | 1.55 |
| Tlast (h) | 7.00 | 4.00 |
| AUC0-last (h * ng/mL) | 180 | 7.1 |
| AUC0-24 (h * ng/mL) | — | — |
| AUC0-inf (h * ng/mL) | 192.0 | 9.1 |
| T½ (h) | 1.24 | 1.31 |

* Median calculated for Tmax and Tlast.

### Example 2-17. DMT Benzamide

| Dosed Test Article: | | DMT Non-Lipid Prodrug 4 |
|---|---|---|
| | | (DMT Benzamide) |
| Dose Route: | | Oral |
| Nominal Dose Concentration: | | 10 mg/Kg |
| Analytes: | Pro-Drug | DMT Non-Lipid Prodrug 4 |
| | Metabolite | DMT |

Chemical name: [3-[2-(dimethylamino)ethyl]indol-1-yl]-phenyl-methanone
Structural class: amide
Mechanistic class: presumed amidases

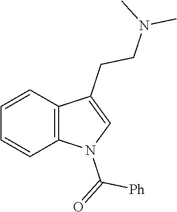 [SMILES: CN(C)CCc1cn(C(=O)c2ccccc2)c2ccccc12]

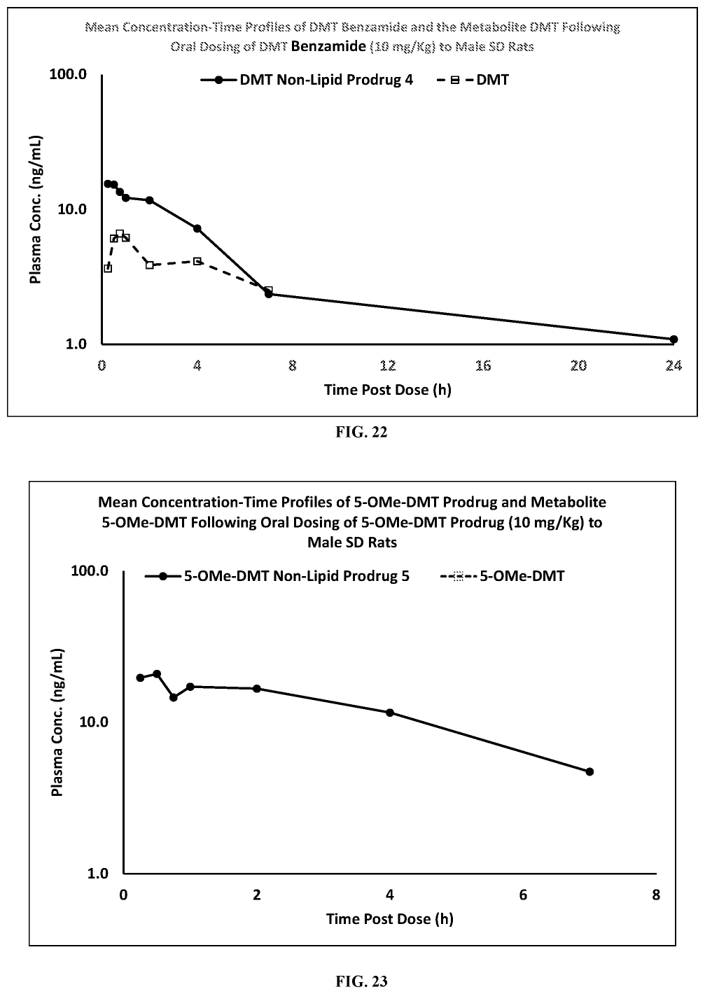

FIG. 22. Mean Concentration-Time Profiles of DMT Benzamide and the Metabolite DMT Following Oral Dosing of DMT Benzamide (10 mg/Kg) to Male SD Rats

TABLE 2-17
DMT Prodrug and DMT PK Parameters
Mean PK Parameters

| PK Parameter | DMT Prodrug | DMT |
|---|---|---|
| Cmax (ng/mL) | 17.7 | 8.62 |
| Tmax (h) | 1.00 | 1.00 |
| MRT (h) | 3.41 | 3.02 |
| Tlast (h) | 7.00 | 7.00 |
| AUC0-last (h * ng/mL) | 66.5 | 27.9 |
| AUC0-24 (h * ng/mL) | — | — |
| AUC0-inf (h * ng/mL) | 104 | 47.3 |
| T½ (h) | 10.3 | 6.36 |

* Median calculated for Tmax and Tlast.

### Example 2-18. 5-MeO-DMT succinate

| | |
|---|---|
| Dosed Test Article: | 5-MeO—DMT Non-Lipid Prodrug 5 |
| | (5-MeO—DMT succinate) |
| Dose Route: | Oral |
| Nominal Dose Concentration: | 10 mg/Kg |
| Analytes: | 5-MeO—DMT Non-Lipid Prodrug 5 |
| | 5-MeO—DMT |

Chemical name: 4-(3-(2-(dimethylamino)ethyl)-5-methoxy-1H-indol-1-yl)-4-oxobutanoic acid
Structural class: amide
Mechanistic class: Presumed pH-dependent cyclization

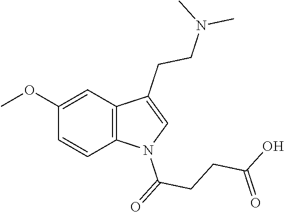 [SMILES: COc1ccc2c(c1)c(CCN(C)C)cn2C(=O)CCC(=O)O]

FIG. 23. Mean Concentration-Time Profiles of 5-MeO-DMT Prodrug and Metabolite 5-MeO-DMT Following Oral Dosing of 5-MeO-DMT Prodrug (10 mg/Kg) to Male SD Rats

TABLE 2-18
5-MeO—DMT Prodrug and 5-MeO—DMT PK parameters
Mean PK Parameters

| | 5-MeO—DMT | |
|---|---|---|
| PK Parameter | Non-Lipid Prodrug 5 | 5-MeO—DMT |
| Cmax (ng/mL) | 23.0 | NC |
| Tmax (h)* | 0.500 | NC |
| MRT (h) | 2.74 | NC |
| Tlast (h)* | 7.00 | NC |
| AUC0-last (h * ng/mL) | 85.7 | NC |
| AUC0-24 (h * ng/mL) | — | NC |
| AUC0-inf (h ng/mL) | — | NC |
| T½ (h) | — | NC |

* Median calculated for Tmax and Tlast.

### Example 2-19. 5-MeO-DMT glutarate

| Dosed Test Article: | | 5-MeO—DMT Non-Lipid Prodrug 6 |
|---|---|---|
| | | (5-MeO—DMT glutarate) |
| Dose Route: | | Oral |
| Nominal Dose Concentration: | | 10 mg/Kg |
| Analytes: | Pro-Drug | 5-MeO—DMT Non-Lipid Prodrug 6 |
| | Metabolite | 5-MeO—DMT |

Chemical name: 5-(3-(2-(dimethylamino)ethyl)-5-methoxy-TH-indol-1-yl)-5-oxopentanoic acid
Structural class: amide
Mechanistic class: Presumed pH-dependent cyclization

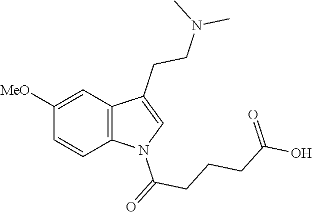 [SMILES: COc1ccc2c(c1)c(CCN(C)C)cn2C(=O)CCCC(=O)O]

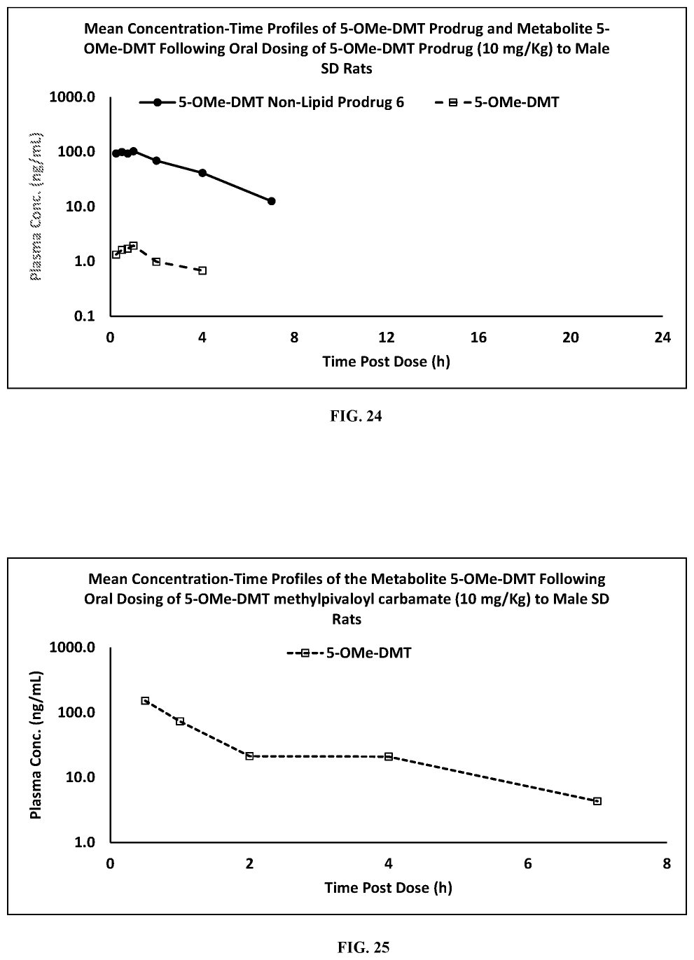

FIG. 24. Mean Concentration-Time Profiles of 5-MeO-DMT Prodrug and Metabolite 5-MeO-DMT Following Oral Dosing of 5-MeO-DMT Prodrug (10 mg/Kg) to Male SD Rats

TABLE 2-19
5-MeO—DMT Prodrug and 5-MeO—DMT PK parameters
Mean PK Parameters

| | 5-MeO—DMT | |
|---|---|---|
| PK Parameter | Non-Lipid Prodrug 6 | 5-MeO—DMT |
| Cmax (ng/mL) | 106 | 2.14 |
| Tmax (h)* | 1.00 | 0.750 |
| MRT (h) | 2.34 | 1.19 |
| Tlast (h)* | 7.00 | 2.00 |
| AUC0-last (h * ng/mL) | 362 | 3.39 |
| AUC0-24 (h * ng/mL) | — | — |
| AUC0-inf (h * ng/mL) | 399 | 7.96 |
| T½ (h) | 2.02 | 2.99 |

*Median calculated for Tmax and Tlast.

### Example 2-20. 5-MeO-DMT methylpivaloyl carbamate

| | |
|---|---|
| Dosed Test Article: | 5-MeO—DMT methylpivaloyl carbamate |
| Dose Route: | Oral |
| Nominal Dose Concentration: | 10 mg/Kg |
| Analyte: | 5-MeO—DMT |

Chemical name: (Pivaloyloxy)methyl 3-(2-(dimethylamino)ethyl)-5-methoxy-1H-indole-1-carboxylate
Structural class: carbamate
Mechanistic class: presumed carboxyesterases+chemical breakdown

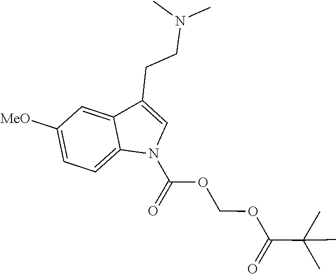 [SMILES: COc1ccc2c(c1)c(CCN(C)C)cn2C(=O)OCOC(=O)C(C)(C)C]

FIG. 25. Mean Concentration-Time Profiles of the Metabolite 5-MeO-DMT Following Oral Dosing of 5-MeO-DMT methylpivaloyl carbamate (10 mg/Kg) to Male SD Rats

TABLE 2-20
5-MeO—DMT PK parameters
Mean* PK Parameters

| PK Parameter | 5-MeO—DMT |
|---|---|
| Cmax (ng/mL) | 152 |
| Tmax (h) | 0.50 |
| MRT (h) | 1.85 |
| Tlast (h) | 7.00 |
| AUC0-last (h * ng/mL) | 222 |
| AUC0-24 (h * ng/mL) | — |
| AUC0-inf (h * ng/mL) | 242.0 |
| T½ (h) | 2.34 |

*Median calculated for Tmax and Tlast.

### Example 2-21. DMT methoxyethyl carbamate

| | |
|---|---|
| Dosed Test Article: | DMT methoxyethyl carbamate formate |
| Dose Route: | Oral |
| Nominal Dose Concentration: | 10 mg/Kg |
| Analyte: | DMT |

Chemical name: 2-methoxyethyl 3-(2-(dimethylamino)ethyl)-1H-indole-1-carboxylate formate
Structural class: carbamate
Mechanistic class: presumed carboxyesterases

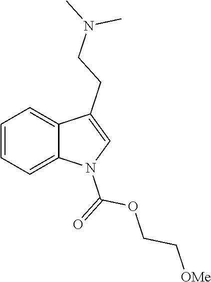 [SMILES: COCCOC(=O)n1cc(CCN(C)C)c2ccccc21]

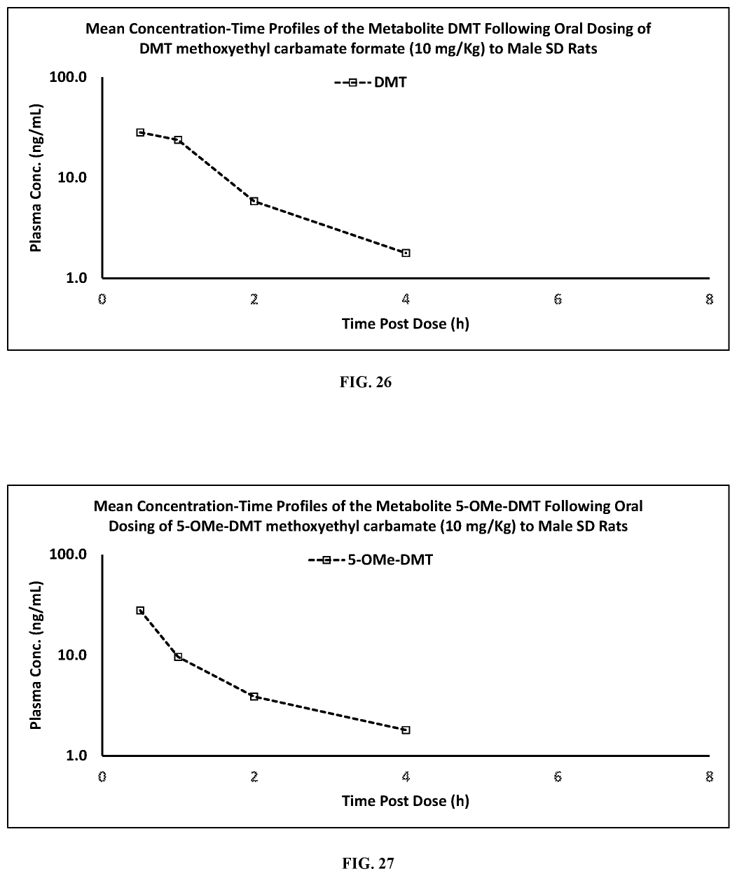

FIG. 26. Mean Concentration-Time Profiles of the Metabolite DMT Following Oral Dosing of DMT methoxyethyl carbamate formate (10 mg/Kg) to Male SD Rats

TABLE 2-21
DMT PK parameters
Mean* PK Parameters

| PK Parameter | DMT |
|---|---|
| Cmax (ng/mL) | 28.70 |
| Tmax (h) | 0.50 |
| MRT (h) | 1.16 |
| Tlast (h) | 4.00 |
| AUC0-last (h * ng/mL) | 42.4 |
| AUC0-24 (h * ng/mL) | — |
| AUC0-inf (h * ng/mL) | 42 |
| T½ (h) | 0.779 |

### Example 2-22. 5-MeO-DMT methoxyethyl carbamate formate

| | |
|---|---|
| Dosed Test Article: | 5-MeO—DMT methoxyethyl carbamate |
| Dose Route: | Oral |
| Nominal Dose Concentration: | 10 mg/Kg |
| Analyte: | 5-MeO—DMT |

Chemical name: 2-methoxyethyl 3-(2-(dimethylamino)ethyl)-5-methoxy-1H-indole-1-carboxylate
Structural class: carbamate
Mechanistic class: presumed carboxyesterases

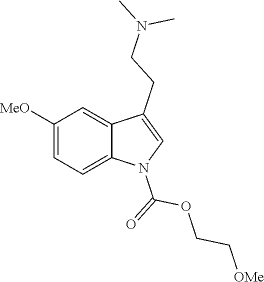 [SMILES: COCCOC(=O)n1cc(CCN(C)C)c2cc(OC)ccc21]

FIG. 27. Mean Concentration-Time Profiles of the Metabolite 5-MeO-DMT Following Oral Dosing of 5-MeO-DMT methoxyethyl carbamate (10 mg/Kg) to Male SD Rats

TABLE 2-22
5-MeO—DMT PK parameters
Mean* PK Parameters

| PK Parameter | 5-MeO—DMT |
|---|---|
| Cmax (ng/mL) | 27.9 |
| Tmax (h) | 0.5 |
| MRT (h) | 0.9 |
| Tlast (h) | 2.0 |
| AUC0-last (h * ng/mL) | 25 |
| AUC0-24 (h * ng/mL) | — |
| AUC0-inf (h * ng/mL) | 37.9 |
| T½ (h) | 1.36 |

*Median calculated for Tmax and Tlast.

### Example 2-23. DMT trimethyl Lock amide

| | |
|---|---|
| Dosed Test Article: | DMT trimethyl lock amide |
| Dose Route: | Oral |
| Nominal Dose Concentration: | 10 mg/Kg |
| Analyte: | DMT |

Chemical name: 2-(4-(3-(2-(dimethylamino)ethyl)-1H-indol-1-yl)-2-methyl-4-oxobutan-2-yl)-3,5-dimethylphenyl acetate
Structural class: amide
Mechanistic class: presumed carboxyesterases+intramolecular cyclization

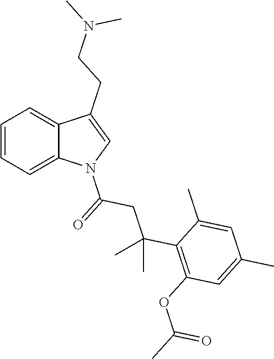 [SMILES: CC(=O)Oc1cc(C)cc(C)c1C(C)(C)CC(=O)n1cc(CCN(C)C)c2ccccc21]

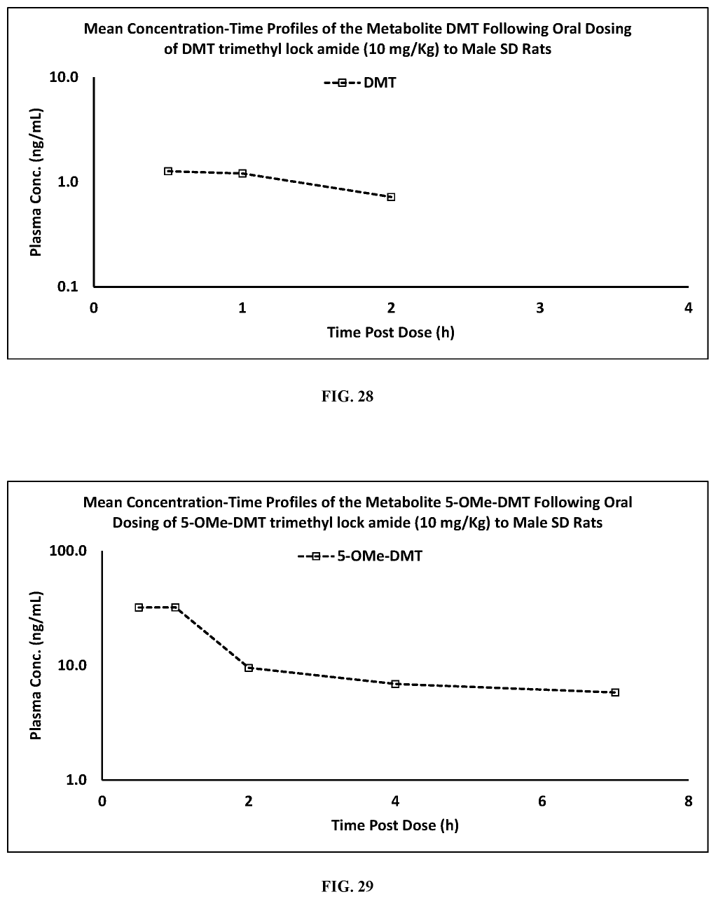

FIG. 28. Mean Concentration-Time Profiles of the Metabolite DMT Following Oral Dosing of DMT trimethyl lock amide (10 mg/Kg) to Male SD Rats

TABLE 2-23
DMT PK parameters
Mean* PK Parameters

| PK Parameter | DMT |
|---|---|
| Cmax (ng/mL) | 1.33 |
| Tmax (h) | 0.50 |
| MRT (h) | 0.80 |
| Tlast (h) | 1.00 |
| AUC0-last (h * ng/mL) | 1.22 |
| AUC0-24 (h * ng/mL) | — |
| AUC0-inf (h * ng/mL) | NC |
| T½ (h) | NC |

*Median calculated for Tmax and Tlast.

### Example 2-24. 5-MeO-DMT trimethyl Lock amide

| | |
|---|---|
| Dosed Test Article: | 5-MeO—DMT trimethyl lock amide |
| Dose Route: | Oral |
| Nominal Dose Concentration: | 10 mg/Kg |
| Analyte: | 5-MeO—DMT |

Chemical name: 2-(4-(3-(2-(dimethylamino)ethyl)-5-methoxy-1H-indol-1-yl)-2-methyl-4-oxobutan-2-yl)-3,5-dimethylphenyl
Structural class: amide
Mechanistic class: presumed carboxyesterases+intramolecular cyclization

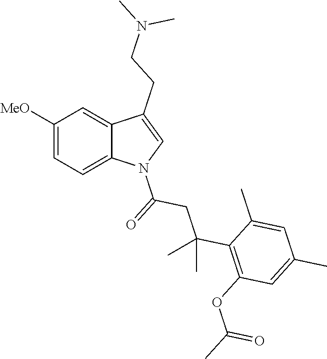 [SMILES: COc1ccc2c(c1)c(CCN(C)C)cn2C(=O)CC(C)(C)c1c(C)cc(C)cc1OC(C)=O]

FIG. 29. Mean Concentration-Time Profiles of the Metabolite 5-MeO-DMT Following Oral Dosing of 5-MeO-DMT trimethyl lock amide (10 mg/Kg) to Male SD Rats

TABLE 2-24
5-MeO—DMT PK parameters
Mean* PK Parameters

| PK Parameter | 5-MeO—DMT |
|---|---|
| Cmax (ng/mL) | 37.70 |
| Tmax (h) | 1.00 |
| MRT (h) | 2.49 |
| Tlast (h) | 7.00 |
| AUC0-last (h * ng/mL) | 80.5 |
| AUC0-24 (h * ng/mL) | — |
| AUC0-inf (h * ng/mL) | 100 |
| T½ (h) | 2.91 |

*Median calculated for Tmax and Tlast.

### Example 2-25. DMT 4-Piperidinopiperidine urea formate

| | |
|---|---|
| Dosed Test Article: | DMT 4-Piperidinopiperidine |
| | urea formate |
| Dose Route: | Oral |
| Nominal Dose Concentration: | 10 mg/Kg |
| Analyte: | DMT |

Chemical name: [1,4′-Bipiperidin]-1′-yl(3-(2-(dimethylamino)ethyl)-1H-indol-1-yl)methanone
Structural class: urea
Mechanistic class: presumed amidases

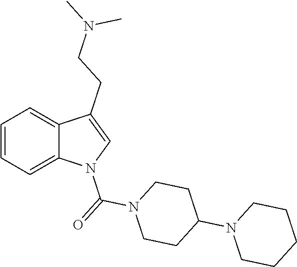 [SMILES: CN(C)CCc1cn(C(=O)N2CCC(N3CCCCC3)CC2)c2ccccc12]

DMT 4-Piperidinopiperidine urea formate
(10 mg/Kg) to Male SD Rats
Mean Plasma Concentration (ng/mL)

| Time (h) | DMT |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-26. 5-MeO-DMT 4-Piperidinopiperidine urea formate

| | |
|---|---|
| Dosed Test Article: | 5-MeO—DMT 4-Piperidinopiperidine |
| | urea formate |
| Dose Route: | Oral |
| Nominal Dose Concentration: | 10 mg/Kg |
| Analyte: | 5-MeO—DMT |

Chemical name: [1,4′-bipiperidin]-1′-yl(3-(2-(dimethylamino)ethyl)-5-methoxy-1H-indol-1-yl)methanone
Structural class: urea
Mechanistic class: presumed amidases

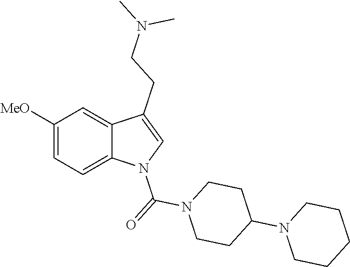 [SMILES: COc1ccc2c(c1)c(CCN(C)C)cn2C(=O)N1CCC(N2CCCCC2)CC1]

FIG. 30. Mean Concentration-Time Profiles of Metabolite 5-MeO-DMT Following Oral Dosing of 5-MeO-DMT 4-Piperidinopiperidine urea formate (10 mg/Kg) to Male SD Rats

TABLE 2-26
5-MeO—DMT PK parameters
Mean* Pharmacokinetic Parameters

| PK Parameter | 5-MeO—DMT |
|---|---|
| Cmax (ng/mL) | 1.14 |
| Tmax (h) | 0.50 |
| MRT (h) | 0.728 |
| Tlast (h) | 1.00 |
| AUC0-last (h * ng/mL) | 1.08 |
| AUC0-24 (h * ng/mL) | — |
| AUC0-inf (h * ng/mL) | NC |
| T½ (h) | NC |

*Median calculated for Tmax and Tlast.
NC: Not Calculated

### Example 2-27. 5-MeO-DMT N,N-dimethyl urea formate

| | |
|---|---|
| Dosed Test Article: | 5-MeO—DMT N,N-dimethyl urea formate |
| Dose Route: | Oral |
| Nominal Dose Concentration: | 10 mg/Kg |
| Analyte: | 5-MeO—DMT |

Chemical name: 3-(2-(dimethylamino)ethyl)-5-methoxy-N,N-dimethyl-1H-indole-1-carboxamide
Structural class: urea
Mechanistic class: presumed amidases

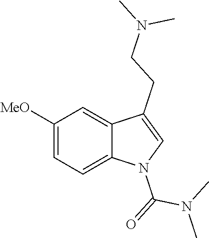 [SMILES: COc1ccc2c(c1)c(CCN(C)C)cn2C(=O)N(C)C]

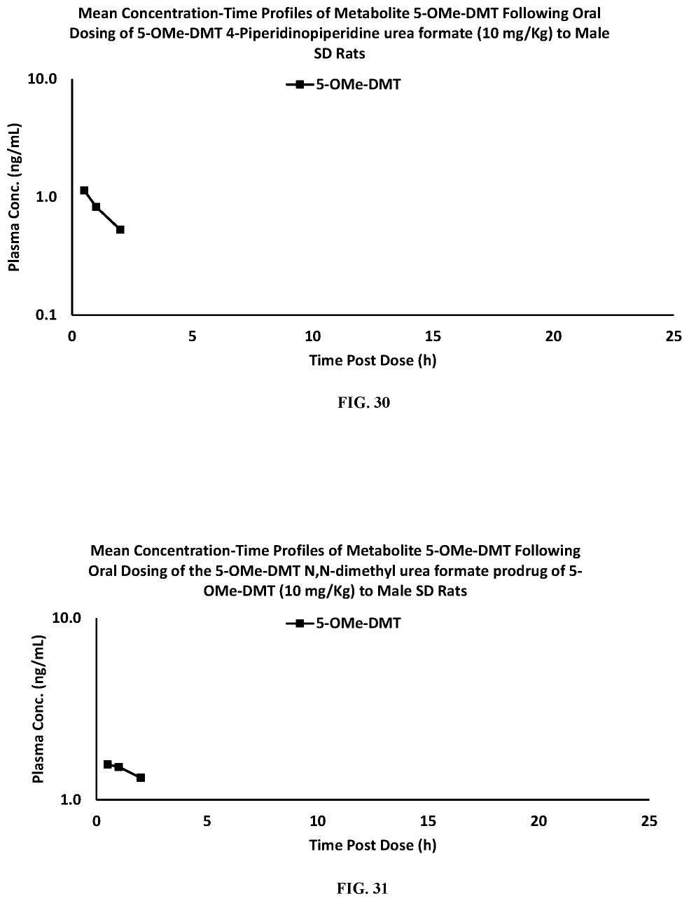

FIG. 31. Mean Concentration-Time Profiles of Metabolite 5-MeO-DMT Following Oral Dosing of the 5-MeO-DMT N,N-dimethyl urea formate prodrug of 5-MeO-DMT (10 mg/Kg) to Male SD Rats

TABLE 2-27
5-MeO-DMT PK parameters
Mean* Pharmacokinetic Parameters

| PK Parameter | 5-MeO—DMT |
|---|---|
| Cmax (ng/mL) | 1.74 |
| Tmax (h) | 1.00 |
| MRT (h) | 1.12 |
| Tlast (h) | 2.00 |
| AUC0-last (h * ng/mL) | 2.58 |
| AUC0-24 (h * ng/mL) | — |
| AUC0-inf (h * ng/mL) | NC |
| T½ (h) | NC |

*Median calculated for Tmax and Tlast.
NC: Not Calculated

### Example 2-28. DMT Lysine tri-hydrochloride

| | |
|---|---|
| Dosed Test Article: | DMT Lysine tri-hydrochloride |
| Dose Route: | Oral |
| Nominal Dose Concentration: | 10 mg/Kg |
| Analyte: | DMT |

Chemical name: (S)-di-tert-butyl (6-(3-(2-(dimethylamino)ethyl)-1H-indol-1-yl)-6-oxohexane-1,5-diyl)dicarbamate
Structural class: amide
Mechanistic class: presumed amidases

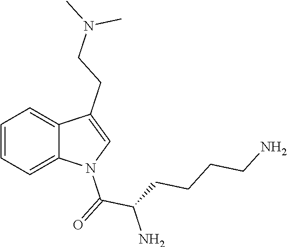 [SMILES: CN(C)CCc1cn(C(=O)[C@@H](N)CCCCN)c2ccccc12]

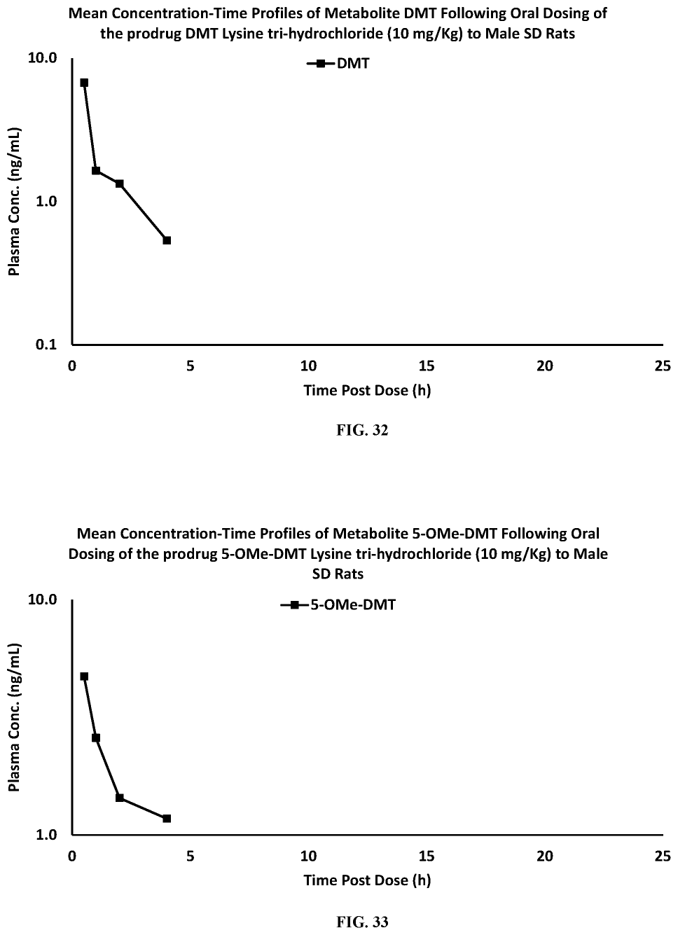

FIG. 32. Mean Concentration-Time Profiles of Metabolite DMT Following Oral Dosing of the prodrug DMT Lysine tri-hydrochloride (10 mg/Kg) to Male SD Rats

TABLE 2-28
DMT PK parameters
Mean* Pharmacokinetic Parameters

| PK Parameter | DMT |
|---|---|
| Cmax (ng/mL) | 6.76 |
| Tmax (h) | 0.50 |
| MRT (h) | 1.17 |
| Tlast (h) | 4.00 |
| AUC0-last (h * ng/mL) | 6.56 |
| AUC0-24 (h * ng/mL) | — |
| AUC0-inf (h * ng/mL) | 9.62 |
| T½ (h) | 1.71 |

*Median calculated for Tmax and Tlast.

### Example 2-29. 5-MeO-DMT Lysine tri-hydrochloride

| | |
|---|---|
| Dosed Test Article: | 5-MeO—DMT Lysine tri-hydrochloride |
| Dose Route: | Oral |
| Nominal Dose Concentration: | 10 mg/Kg |
| Analyte: | 5-MeO—DMT |

Chemical name: (S)-2,6-diamino-1-(3-(2-(dimethylamino)ethyl)-5-methoxy-1H-indol-1-yl)hexan-1-one
Structural class: amide
Mechanistic class: presumed amidases

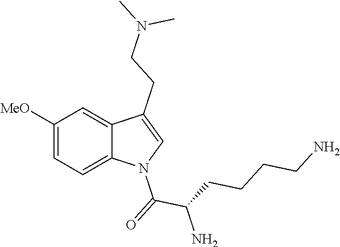 [SMILES: COc1ccc2c(c1)c(CCN(C)C)cn2C(=O)[C@@H](N)CCCCN]

FIG. 33. Mean Concentration-Time Profiles of Metabolite 5-MeO-DMT Following Oral Dosing of the prodrug 5-MeO-DMT Lysine tri-hydrochloride (10 mg/Kg) to Male SD Rats

TABLE 2-29
5-MeO—DMT PK parameters
Mean* Pharmacokinetic Parameters

| PK Parameter | 5-MeO—DMT |
|---|---|
| Cmax (ng/mL) | 4.72 |
| Tmax (h) | 0.50 |
| MRT (h) | 1.57 |
| Tlast (h) | 4.00 |
| AUC0-last (h * ng/mL) | 7.63 |
| AUC0-24 (h * ng/mL) | — |
| AUC0-inf (h * ng/mL) | 13.4 |
| T½ (h) | 3.16 |

*Median calculated for Tmax and Tlast.

### Example 2-30. Di-DMT urea (Symmetrical urea) di-formate salt

| | |
|---|---|
| Dosed Test Article: | Di-DMT urea |
| | (symmetrical urea) di-formate salt |
| Dose Route: | Oral |
| Nominal Dose Concentration: | 10 mg/Kg |
| Analyte: | DMT |

Chemical name: bis(3-(2-(dimethylamino)ethyl)-1H-indol-1-yl)methanone
Structural class: symmetrical dimer (urea)
Mechanistic class: presumed amidases

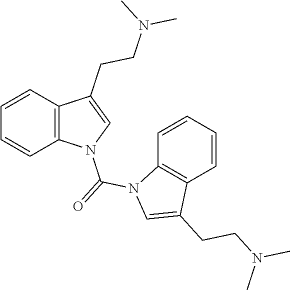 [SMILES: CN(C)CCc1cn(C(=O)n2cc(CCN(C)C)c3ccccc32)c2ccccc12]

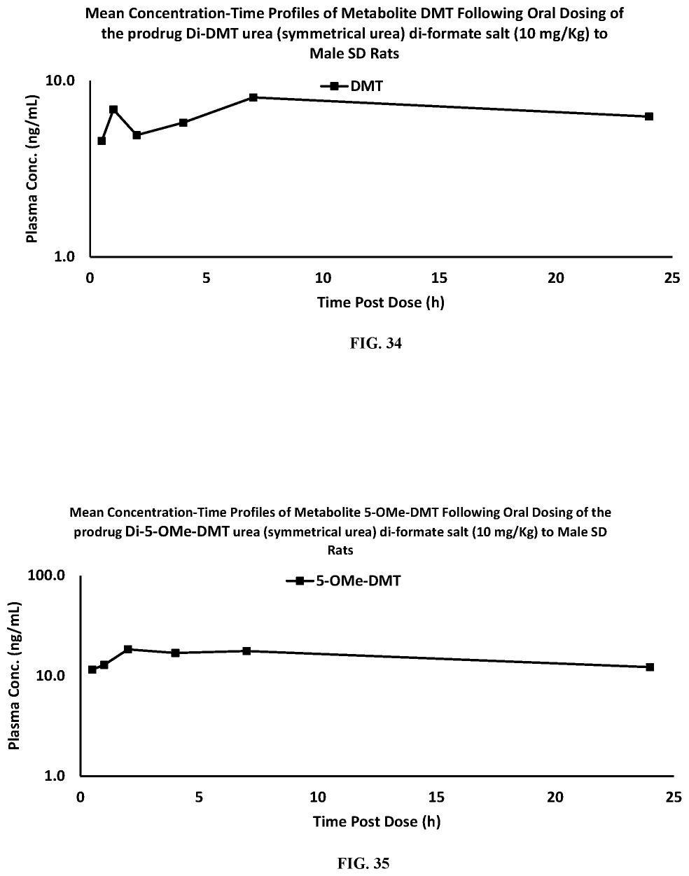

FIG. 34. Mean Concentration-Time Profiles of Metabolite DMT Following Oral Dosing of the prodrug Di-DMT urea (symmetrical urea) di-formate salt (10 mg/Kg) to Male SD Rats

TABLE 2-30
DMT PK parameters
Mean* Pharmacokinetic Parameters

| PK Parameter | DMT |
|---|---|
| Cmax (ng/mL) | 8.04 |
| Tmax (h) | 7.00 |
| MRT (h) | 11.8 |
| Tlast (h) | 24.0 |
| AUC0-last (h * ng/mL) | 163 |
| AUC0-24 (h * ng/mL) | 163 |
| AUC0-inf (h * ng/mL) | NC |
| T½ (h) | NC |

*Median calculated for Tmax and Tlast.

### Example 2-31. Di-5-MeO-DMT urea-(Symmetrical urea) di-formate salt

| | |
|---|---|
| Dosed Test Article: | Di-5-MeO—DMT urea |
| | (symmetrical urea) di-formate salt |
| Dose Route: | Oral |
| Nominal Dose Concentration: | 10 mg/Kg |
| Analyte: | 5-MeO—DMT |

Chemical name: bis(3-(2-(dimethylamino)ethyl)-5-methoxy-1H-indol-1-yl)methanone
Structural class: symmetrical dimer (urea)
Mechanistic class: presumed amidases

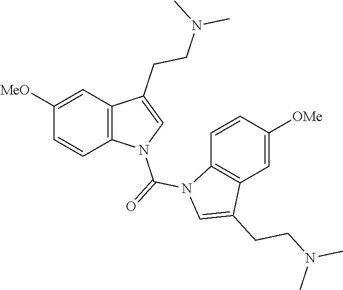 [SMILES: COc1ccc2c(c1)c(CCN(C)C)cn2C(=O)n1cc(CCN(C)C)c2cc(OC)ccc21]

FIG. 35. Mean Concentration-Time Profiles of Metabolite 5-MeO-DMT Following Oral Dosing of the prodrug Di-5-MeO-DMT urea (symmetrical urea) di-formate salt (10 mg/Kg) to Male SD Rats

TABLE 2-31
5-MeO—DMT PK parameters
Mean* Pharmacokinetic Parameters

| PK Parameter | 5-MeO—DMT |
|---|---|
| Cmax (ng/mL) | 19.1 |
| Tmax (h) | 2.00 |
| MRT (h) | 10.9 |
| Tlast (h) | 24.0 |
| AUC0-last (h * ng/mL) | 366 |
| AUC0-24 (h * ng/mL) | 366 |
| AUC0-inf (h * ng/mL) | 995 |
| T½ (h) | 33.3 |

*Median calculated for Tmax and Tlast.

### Example 32. DMT Valine di-hydrochloride

| | |
|---|---|
| Dosed Test Article: | DMT Valine di-hydrochloride |
| Dose Route: | Oral |
| Nominal Dose Concentration: | 10 mg/Kg |
| Analyte: | DMT |

Chemical name: (S)-2-amino-1-(3-(2-(dimethylamino)ethyl)-1H-indol-1-yl)-3-methylbutan-1-one
Structural class: amide
Mechanistic class: presumed amidases

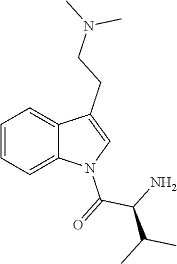 [SMILES: CC(C)[C@H](N)C(=O)n1cc(CCN(C)C)c2ccccc21]

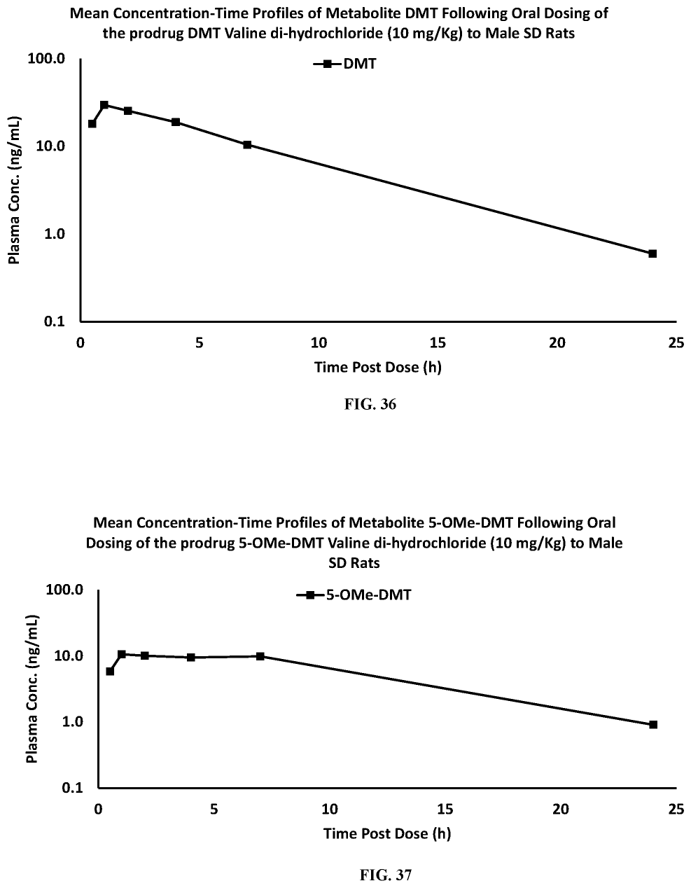

FIG. 36. Mean Concentration-Time Profiles of Metabolite DMT Following Oral Dosing of the prodrug DMT Valine di-hydrochloride (10 mg/Kg) to Male SD Rats

TABLE 2-32
DMT PK parameters
Mean* Pharmacokinetic Parameters

| PK Parameter | DMT |
|---|---|
| Cmax (ng/mL) | 31.1 |
| Tmax (h) | 1.00 |
| MRT (h) | 3.73 |
| Tlast (h) | 7.00 |
| AUC0-last (h * ng/mL) | 174 |
| AUC0-24 (h * ng/mL) | 268 |
| AUC0-inf (h * ng/mL) | 205 |
| T½ (h) | 3.69 |

*Median calculated for Tmax and Tlast.

### Example 2-33. 5-MeO-DMT Valine di-hydrochloride

| | |
|---|---|
| Dosed Test Article: | 5-MeO—DMT Valine di-hydrochloride |
| Dose Route: | Oral |
| Nominal Dose Concentration: | 10 mg/Kg |
| Analyte: | 5-MeO—DMT |

Chemical name: (S)-2-amino-1-(3-(2-(dimethylamino)ethyl)-5-methoxy-1H-indol-1-yl)-3-methylbutan-1-one
Structural class: amide
Mechanistic class: presumed amidases

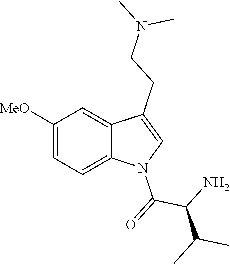 [SMILES: COc1ccc2c(c1)c(CCN(C)C)cn2C(=O)[C@@H](N)C(C)C]

FIG. 37. Mean Concentration-Time Profiles of Metabolite 5-MeO-DMT Following Oral Dosing of the prodrug 5-MeO-DMT Valine di-hydrochloride (10 mg/Kg) to Male SD Rats

TABLE 2-33
5-MeO—DMT PK parameters
Mean* Pharmacokinetic Parameters

| PK Parameter | 5-MeO—DMT |
|---|---|
| Cmax (ng/mL) | 11.1 |
| Tmax (h) | 2.00 |
| MRT (h) | 6.47 |
| Tlast (h) | 24.0 |
| AUC0-last (h * ng/mL) | 155 |
| AUC0-24 (h * ng/mL) | 155 |
| AUC0-inf (h * ng/mL) | 150 |
| T½ (h) | 5.54 |

*Median calculated for Tmax and Tlast.

### Example 2-34. 5-MeO-DMT N,N-dimethylglycine formate

| | |
|---|---|
| Dosed Test Article: | 5-MeO—DMT N,N- |
| | dimethylglycine formate |
| Dose Route: | Oral |
| Nominal Dose Concentration: | 10 mg/Kg |
| Analyte: | 5-MeO—DMT |

Chemical name: 2-(dimethylamino)-1-(3-(2-(dimethylamino)ethyl)-5-methoxy-1H-indol-1-yl)ethan-1-one
Structural class: amide
Mechanistic class: presumed amidases

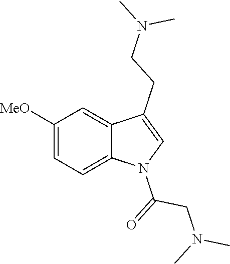 [SMILES: COc1ccc2c(c1)c(CCN(C)C)cn2C(=O)CN(C)C]

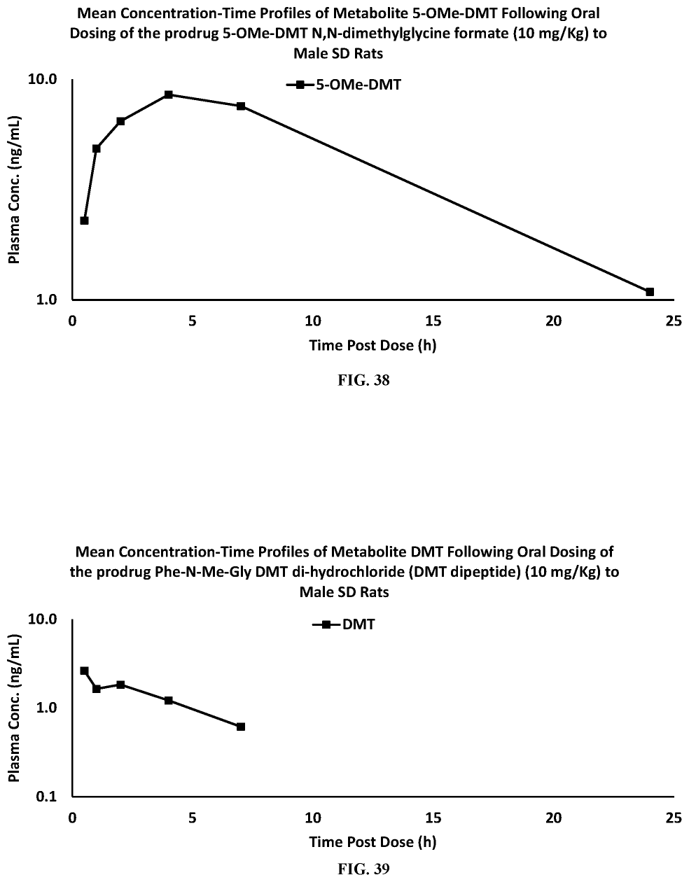

FIG. 38. Mean Concentration-Time Profiles of Metabolite 5-MeO-DMT Following Oral Dosing of the prodrug 5-MeO-DMT N,N-dimethylglycine formate (10 mg/Kg) to Male SD Rats

TABLE 2-34
5-MeO—DMT PK parameters
Mean* Pharmacokinetic Parameters

| PK Parameter | 5-MeO—DMT |
|---|---|
| Cmax (ng/mL) | 8.58 |
| Tmax (h) | 4.00 |
| MRT (h) | 7.13 |
| Tlast (h) | 24.0 |
| AUC0-last (h * ng/mL) | 121 |
| AUC0-24 (h * ng/mL) | 121 |
| AUC0-inf (h * ng/mL) | 134 |
| T½ (h) | 6.46 |

*Median calculated for Tmax and Tlast.

### Example 2-35. Phe-N-Me-Gly DMT di-hydrochloride (DMT Dipeptide)

| | |
|---|---|
| Dosed Test Article: | Phe-N—Me-Gly DMT di-hydrochloride |
| | (DMT dipeptide) |
| Dose Route: | Oral |
| Nominal Dose Concentration: | 10 mg/Kg |
| Analyte: | DMT |

Chemical name: (S)-2-amino-N-(2-(3-(2-(dimethylamino)ethyl)-1H-indol-1-yl)-2-oxoethyl)-N-methyl-3-phenylpropanamide
Structural class: amide
Mechanistic class: pH-dependent cyclization

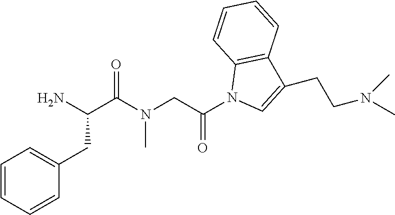 [SMILES: CN(C)CCc1cn(C(=O)CN(C)C(=O)[C@@H](N)Cc2ccccc2)c2ccccc12]

FIG. 39. Mean Concentration-Time Profiles of Metabolite DMT Following Oral Dosing of the prodrug Phe-N-Me-Gly DMT di-hydrochloride (DMT dipeptide) (10 mg/Kg) to Male SD Rats

TABLE 2-35
DMT PK parameters
Mean* Pharmacokinetic Parameters

| PK Parameter | DMT |
|---|---|
| Cmax (ng/mL) | 2.43 |
| Tmax (h) | 0.50 |
| MRT (h) | 2.30 |
| Tlast (h) | 4.00 |
| AUC0-last (h * ng/mL) | 7.17 |
| AUC0-24 (h * ng/mL) | — |
| AUC0-inf (h * ng/mL) | 12.0 |
| T½ (h) | 3.31 |

*Median calculated for Tmax and Tlast.

### Example 2-36. DMT Alanine di-hydrochloride

| | |
|---|---|
| Dosed Test Article: | DMT Alanine di-hydrochloride |
| Dose Route: | Oral |
| Nominal Dose Concentration: | 10 mg/Kg |
| Analyte: | DMT |

Chemical name: (S)-2-amino-1-(3-(2-(dimethylamino)ethyl)-1H-indol-1-yl)propan-1-one
Structural class: amide
Mechanistic class: presumed amidases

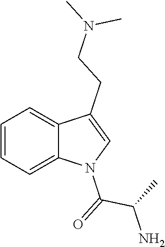 [SMILES: C[C@H](N)C(=O)n1cc(CCN(C)C)c2ccccc21]

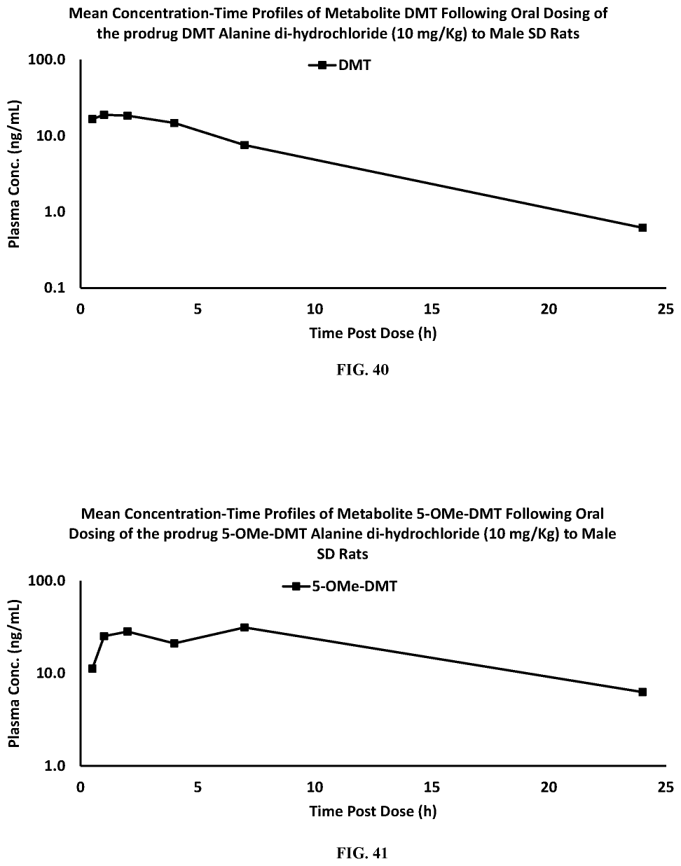

FIG. 40. Mean Concentration-Time Profiles of Metabolite DMT Following Oral Dosing of the prodrug DMT Alanine di-hydrochloride (10 mg/Kg) to Male SD Rats

TABLE 2-36
DMT PK parameters
Mean* Pharmacokinetic Parameters

| PK Parameter | DMT |
|---|---|
| Cmax (ng/mL) | 20.2 |
| Tmax (h) | 0.50 |
| MRT (h) | 3.73 |
| Tlast (h) | 7.00 |
| AUC0-last (h * ng/mL) | 126 |
| AUC0-24 (h * ng/mL) | 195 |
| AUC0-inf (h * ng/mL) | 153 |
| T½ (h) | 4.08 |

*Median calculated for Tmax and Tlast.

### Example 2-37. 5-MeO-DMT Alanine di-hydrochloride

| | |
|---|---|
| Dosed Test Article: | 5-MeO—DMT Alanine di-hydrochloride |
| Dose Route: | Oral |
| Nominal Dose Concentration: | 10 mg/Kg |
| Analyte: | 5-MeO—DMT |

Chemical name: (S)-2-amino-1-(3-(2-(dimethylamino)ethyl)-5-methoxy-1H-indol-1-yl)propan-1-one
Structural class: amide
Mechanistic class: presumed amidases

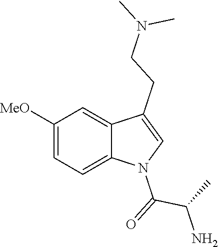 [SMILES: COc1ccc2c(c1)c(CCN(C)C)cn2C(=O)[C@H](C)N]

FIG. 41. Mean Concentration-Time Profiles of Metabolite 5-MeO-DMT Following Oral Dosing of the prodrug 5-MeO-DMT Alanine di-hydrochloride (10 mg/Kg) to Male SD Rats

TABLE 2-37
5-MeO—DMT PK parameters
Mean* Pharmacokinetic Parameters

| PK Parameter | 5-MeO—DMT |
|---|---|
| Cmax (ng/mL) | 34.7 |
| Tmax (h) | 2.00 |
| MRT (h) | 7.78 |
| Tlast (h) | 24.0 |
| AUC0-last (h * ng/mL) | 488 |
| AUC0-24 (h * ng/mL) | 488 |
| AUC0-inf (h * ng/mL) | 630 |
| T½ (h) | 10.0 |

*Median calculated for Tmax and Tlast.

### Example 2-38. DMT tetramethylphosphorodiamide

| | |
|---|---|
| Dosed Test Article: | DMT tetramethylphosphorodiamide |
| Dose Route: | Oral |
| Nominal Dose Concentration: | 10 mg/Kg |
| Analyte: | DMT |

Chemical name: 2-(1-di(dimethylamino)phosphoryl-indol-3-yl)-N,N-dimethyl-ethanamine
Structural class: phosphorodiamidate prodrug
Mechanistic class: presumed phosphatase

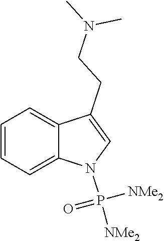 [SMILES: CN(C)CCc1cn(P(=O)(N(C)C)N(C)C)c2ccccc12]

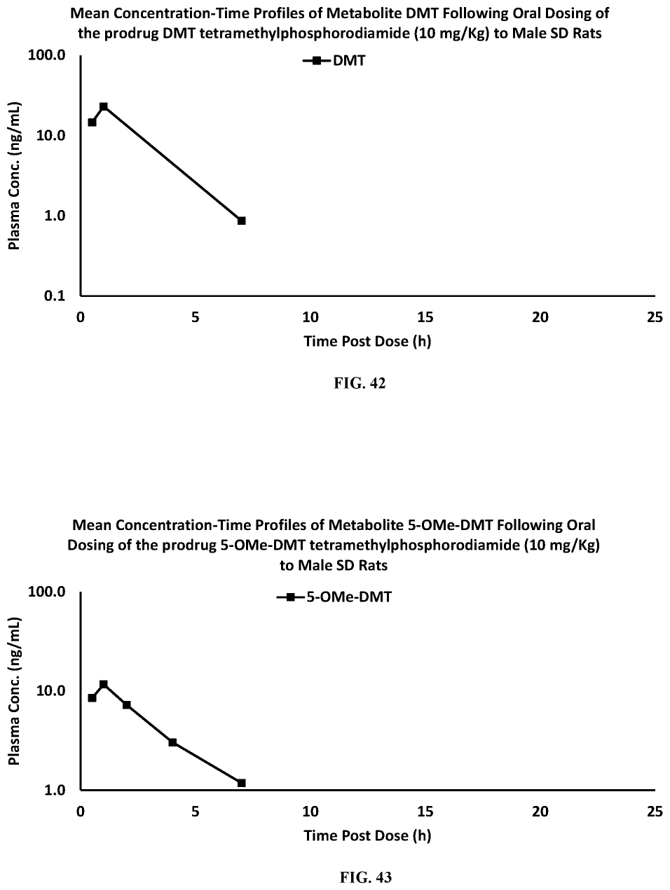

FIG. 42. Mean Concentration-Time Profiles of Metabolite DMT Following Oral Dosing of the prodrug DMT tetramethylphosphorodiamide (10 mg/Kg) to Male SD Rats

TABLE 2-38
DMT PK Parameters
Mean* Pharmacokinetic Parameters

| PK Parameter | DMT |
|---|---|
| Cmax (ng/mL) | 23.0 |
| Tmax (h) | 1.00 |
| MRT (h) | 1.14 |
| Tlast (h) | 7.00 |
| AUC0-last (h * ng/mL) | 84.7 |
| AUC0-24 (h * ng/mL) | NC |
| AUC0-inf (h * ng/mL) | NC |
| T½ (h) | NC |

*Median calculated for Tmax and Tlast.
NC: Not Calculated

### Example 2-39. 5-MeO-DMT tetramethylphosphorodiamide

| | |
|---|---|
| Dosed Test Article: | 5-MeO—DMT |
| | tetramethylphosphorodiamide |
| Dose Route: | Oral |
| Nominal Dose Concentration: | 10 mg/Kg |
| Analyte: | 5-MeO—DMT |

Chemical name: 2-(1-di(dimethylamino)phosphoryl-5-methoxy-indol-3-yl)-N,N-dimethyl-ethanamine
Structural class: phosphorodiamidate prodrug
Mechanistic class: presumed phosphatase

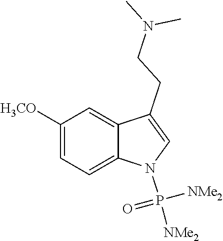 [SMILES: COc1ccc2c(c1)c(CCN(C)C)cn2P(=O)(N(C)C)N(C)C]

FIG. 43. Mean Concentration-Time Profiles of Metabolite 5-MeO-DMT Following Oral Dosing of the prodrug 5-MeO-DMT tetramethylphosphorodiamide (10 mg/Kg) to Male SD Rats

TABLE 2-39
5-MeO—DMT PK Parameters
Mean* Pharmacokinetic Parameters

| PK Parameter | 5-MeO—DMT |
|---|---|
| Cmax (ng/mL) | 11.7 |
| Tmax (h) | 1.00 |
| MRT (h) | 2.28 |
| Tlast (h) | 7.00 |
| AUC0-last (h * ng/mL) | 33.3 |
| AUC0-24 (h * ng/mL) | NC |
| AUC0-inf (h * ng/mL) | 36.6 |
| T½ (h) | 1.96 |

*Median calculated for Tmax and Tlast.
NC: Not Calculated

### Example 2-40. DMT Phenylalanine di-hydrochloride

| | |
|---|---|
| Dosed Test Article: | DMT Phenylalanine di-hydrochloride |
| Dose Route: | Oral |
| Nominal Dose Concentration: | 10 mg/Kg |
| Analyte: | DMT |

Chemical name: (S)-2-amino-1-(3-(2-(dimethylamino)ethyl)-1H-indol-1-yl)-3-phenylpropan-1-one bis-hydrochloride
Structural class: amino acid prodrug
Mechanistic class: presumed amidase

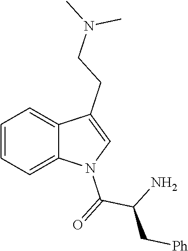 [SMILES: CN(C)CCc1cn(C(=O)[C@@H](N)Cc2ccccc2)c2ccccc12]

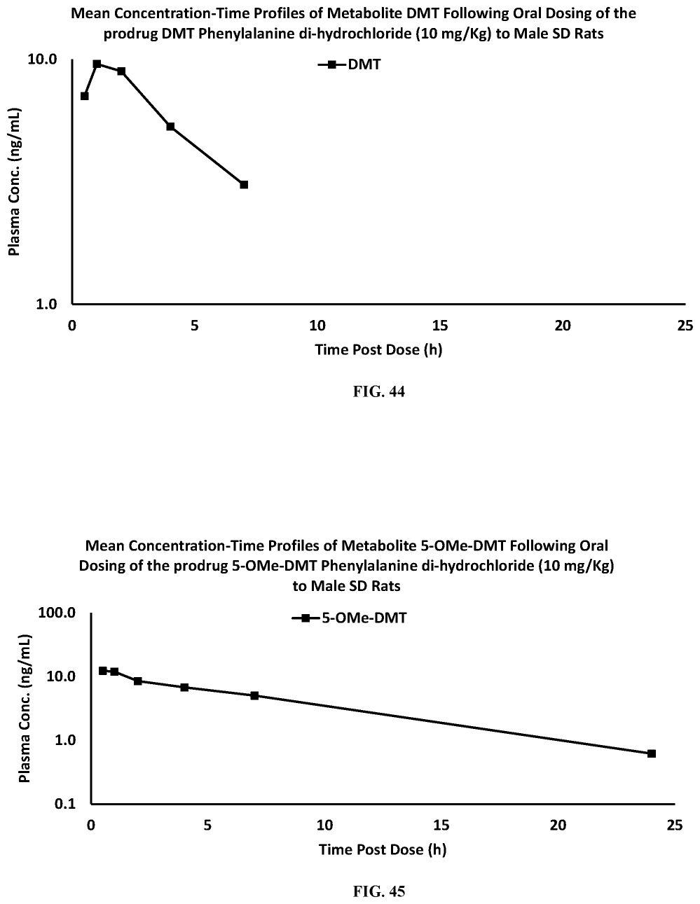

FIG. 44. Mean Concentration-Time Profiles of Metabolite DMT Following Oral Dosing of the prodrug DMT Phenylalanine di-hydrochloride (10 mg/Kg) to Male SD Rats

TABLE 2-40
DMT PK Parameters
Mean* Pharmacokinetic Parameters

| PK Parameter | DMT |
|---|---|
| Cmax (ng/mL) | 10.2 |
| Tmax (h) | 1.00 |
| MRT (h) | 2.93 |
| Tlast (h) | 7.00 |
| AUC0-last (h * ng/mL) | 42.0 |
| AUC0-24 (h * ng/mL) | NC |
| AUC0-inf (h * ng/mL) | 76.7 |
| T½ (h) | 6.38 |

*Median calculated for Tmax and Tlast.
NC: Not Calculated

### Example 2-41. 5-MeO-DMT Phenylalanine di-hydrochloride

| | |
|---|---|
| Dosed Test Article: | 5-MeO—DMT Phenylalanine |
| | di-hydrochloride |
| Dose Route: | Oral |
| Nominal Dose Concentration: | 10 mg/Kg |
| Analyte: | 5-MeO—DMT |

Chemical name: (S)-2-amino-1-(3-(2-(dimethylamino)ethyl)-5-methoxy-1H-indol-1-yl)-3-phenylpropan-1-one bis-hydrochloride
Structural class: amino acid prodrug
Mechanistic class: presumed amidase

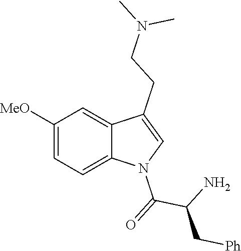 [SMILES: COc1ccc2c(c1)c(CCN(C)C)cn2C(=O)[C@@H](N)Cc1ccccc1]

FIG. 45. Mean Concentration-Time Profiles of Metabolite 5-MeO-DMT Following Oral Dosing of the prodrug 5-MeO-DMT Phenylalanine di-hydrochloride (10 mg/Kg) to Male SD Rats

TABLE 41
5-MeO—DMT PK Parameters
Mean* Pharmacokinetic Parameters

| PK Parameter | 5-MeO—DMT |
|---|---|
| Cmax (ng/mL) | 13.3 |
| Tmax (h) | 0.50 |
| MRT (h) | 4.85 |
| Tlast (h) | 24.0 |
| AUC0-last (h * ng/mL) | 86.2 |
| AUC0-24 (h * ng/mL) | 104 |
| AUC0-inf (h * ng/mL) | 99.8 |
| T½ (h) | 5.37 |

*Median calculated for Tmax and Tlast.

### Example 2-42. 5-MeO-DMT 2,2-dimethylpropyl pivalate carbamate formate

| | |
|---|---|
| Dosed Test Article: | 5-MeO—DMT 2,2-dimethylpropyl |
| | pivalate carbamate formate |
| Dose Route: | Oral |
| Nominal Dose Concentration: | 10 mg/Kg |
| Analyte: | 5-MeO—DMT |

Chemical name: 2,2-dimethyl-3-(pivaloyloxy)propyl 3-(2-(dimethylamino)ethyl)-5-methoxy-1H-indole-1-carboxylate formate
Structural class: carbamate prodrug
Mechanistic class: presumed carboxyesterases+intramolecular cyclization

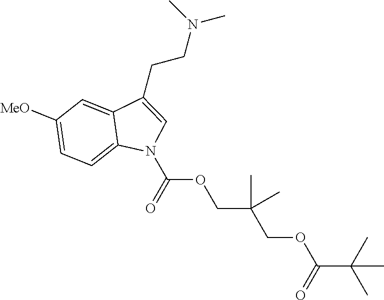 [SMILES: COc1ccc2c(c1)c(CCN(C)C)cn2C(=O)OCC(C)(C)COC(=O)C(C)(C)C]

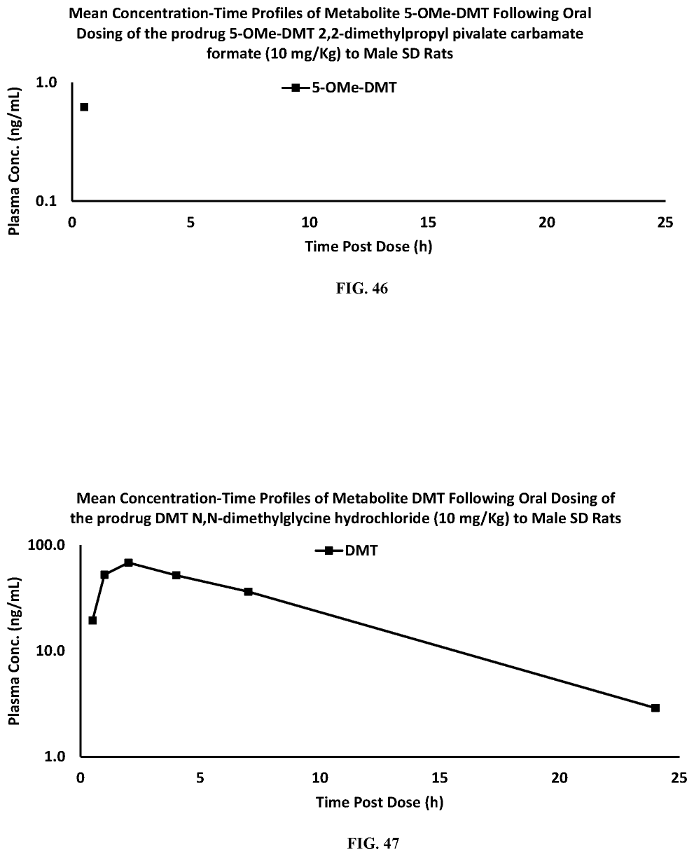

FIG. 46. Mean Concentration-Time Profiles of Metabolite 5-MeO-DMT Following Oral Dosing of the prodrug 5-MeO-DMT 2,2-dimethylpropyl pivalate carbamate formate (10 mg/Kg) to Male SD Rats

TABLE 2-42
5-MeO—DMT PK Parameters
Mean* Pharmacokinetic Parameters

| PK Parameter | 5-MeO—DMT |
|---|---|
| Cmax (ng/mL) | 0.623 |
| Tmax (h) | 0.50 |
| MRT (h) | 0.50 |
| Tlast (h) | 0.50 |
| AUC0-last (h * ng/mL) | 0.156 |
| AUC0-24 (h * ng/mL) | NC |
| AUC0-inf (h * ng/mL) | NC |
| T½ (h) | NC |

*Median calculated for Tmax and Tlast.
NC: Not Calculated

### Example 2-43. DMT N,N-dimethylglycine hydrochloride

| | |
|---|---|
| Dosed Test Article: | DMT N,N-dimethylglycine hydrochloride |
| Dose Route: | Oral |
| Nominal Dose Concentration: | 10 mg/Kg |
| Analyte: | DMT |

Chemical name: 2-(dimethylamino)-1-(3-(2-(dimethylamino)ethyl)-1H-indol-1-yl)ethan-1-one hydrochloride
Structural class: amino acid prodrug
Mechanistic class: presumed amidase

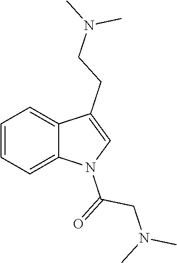 [SMILES: CN(C)CCc1cn(C(=O)CN(C)C)c2ccccc12]

FIG. 47. Mean Concentration-Time Profiles of Metabolite DMT Following Oral Dosing of the prodrug DMT N,N-dimethylglycine hydrochloride (10 mg/Kg) to Male SD Rats

TABLE 2-43
DMT PK Parameters
Mean* Pharmacokinetic Parameters

| PK Parameter | DMT |
|---|---|
| Cmax (ng/mL) | 68.4 |
| Tmax (h) | 2.00 |
| MRT (h) | 5.84 |
| Tlast (h) | 24.0 |
| AUC0-last (h * ng/mL) | 670 |
| AUC0-24 (h * ng/mL) | 670 |
| AUC0-inf (h * ng/mL) | 690 |
| T½ (h) | 4.78 |

*Median calculated for Tmax and Tlast.

### Example 2-44. DMT N,N-dimethyl urea formate

| | |
|---|---|
| Dosed Test Article: | DMT N,N-dimethyl urea formate |
| Dose Route: | Oral |
| Nominal Dose Concentration: | 10 mg/Kg |
| Analyte: | DMT |

Chemical name: 3-[2-(dimethylamino)ethyl]-N,N-dimethyl-indole-1-carboxamide
Structural class: urea prodrug
Mechanistic class: presumed amidase

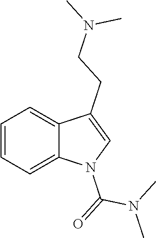 [SMILES: CN(C)CCc1cn(C(=O)N(C)C)c2ccccc12]

TABLE 2-44
Mean Concentration-Time Profiles of Metabolite DMT
Following Oral Dosing of the prodrug DMT N,N-dimethyl
urea formate (10 mg/Kg) to Male SD Rats
Mean Plasma Concentrations (ng/mL)

| Time (h) | DMT |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-45. DMT methyl pivalate

| | |
|---|---|
| Dosed Test Article: | DMT methyl pivalate |
| Dose Route: | Oral |
| Nominal Dose Concentration: | 10 mg/Kg |
| Analyte: | DMT |

Chemical name: (3-(2-(dimethylamino)ethyl)-1H-indol-1-yl)methyl pivalate
Structural class: acyloxymethyl prodrug
Mechanistic class: presumed carboxyesterase+chemical breakdown

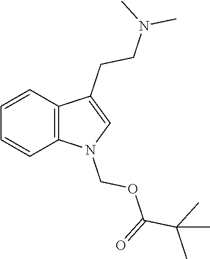 [SMILES: CN(C)CCc1cn(COC(=O)C(C)(C)C)c2ccccc12]

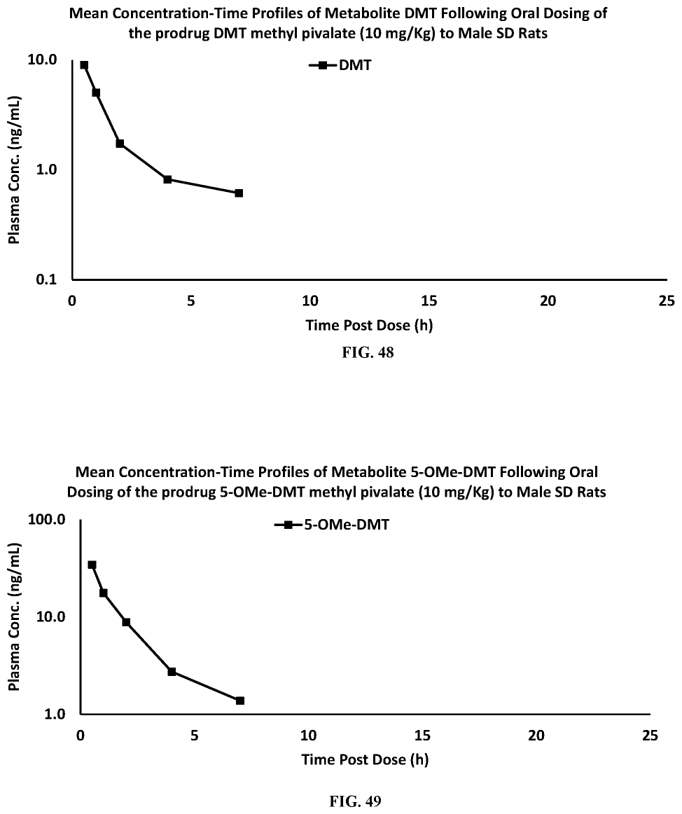

FIG. 48. Mean Concentration-Time Profiles of Metabolite DMT Following Oral Dosing of the prodrug DMT methyl pivalate (10 mg/Kg) to Male SD Rats

TABLE 2-45
DMT PK Parameters
Mean* Pharmacokinetic Parameters

| PK Parameter | DMT |
|---|---|
| Cmax (ng/mL) | 9.02 |
| Tmax (h) | 0.50 |
| MRT (h) | 1.17 |
| Tlast (h) | 4.00 |
| AUC0-last (h * ng/mL) | 10.2 |
| AUC0-24 (h * ng/mL) | NC |
| AUC0-inf (h * ng/mL) | 17.0 |
| T½ (h) | 2.04 |

*Median calculated for Tmax and Tlast.
NC: Not Calculated

### Example 2-46. 5-MeO-DMT methyl pivalate

| | |
|---|---|
| Dosed Test Article: | 5-MeO—DMT methyl pivalate |
| Dose Route: | Oral |
| Nominal Dose Concentration: | 10 mg/Kg |
| Analyte: | 5-MeO—DMT |

Chemical name: (3-(2-(Dimethylamino)ethyl)-5-methoxy-1H-indol-1-yl)methyl pivalate
Structural class: acyloxymethyl prodrug
Mechanistic class: presumed carboxyesterase+chemical breakdown

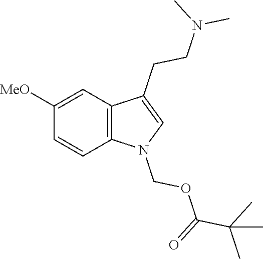 [SMILES: COc1ccc2c(c1)c(CCN(C)C)cn2COC(=O)C(C)(C)C]

FIG. 49. Mean Concentration-Time Profiles of Metabolite 5-MeO-DMT Following Oral Dosing of the prodrug 5-MeO-DMT methyl pivalate (10 mg/Kg) to Male SD Rats

TABLE 2-46
5-MeO—DMT PK Parameters
Mean* Pharmacokinetic Parameters

| PK Parameter | 5-MeO—DMT |
|---|---|
| Cmax (ng/mL) | 34.4 |
| Tmax (h) | 0.50 |
| MRT (h) | 1.41 |
| Tlast (h) | 4.00 |
| AUC0-last (h * ng/mL) | 48.8 |
| AUC0-24 (h * ng/mL) | — |
| AUC0-inf (h * ng/mL) | 53.3 |
| T½ (h) | 1.34 |

*Median calculated for Tmax and Tlast.
NC: Not Calculated

### Example 2-47. DMT-3,3-dimethylsuccinate hydrochloride

| | |
|---|---|
| Dosed Test Article: | DMT-3,3-dimethylsuccinate hydrochloride |
| Dose Route: | Oral |
| Nominal Dose Concentration: | 10 mg/Kg |
| Analyte: | DMT |

Chemical name: 4-(3-(2-(dimethylamino)ethyl)-1H-indol-1-yl)-2,2-dimethyl-4-oxobutanoic acid HCl salt
Structural class: amide prodrug
Mechanistic class: presumed pH-dependent cyclization

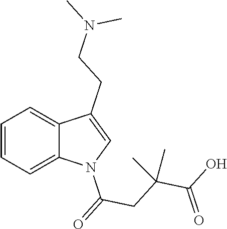 [SMILES: CN(C)CCc1cn(C(=O)CC(C)(C)C(=O)O)c2ccccc12]

TABLE 2-47
Mean Concentration-Time Profiles of Metabolite DMT
Following Oral Dosing of the prodrug DMT-3,3-dimethylsuccinate
hydrochloride (10 mg/Kg) to Male SD Rats
Mean Plasma Concentrations (ng/mL)

| Time (h) | DMT |
|---|---|
| 0.50 | BLQ |
| 1.00 | BLQ |
| 2.00 | BLQ |
| 4.00 | BLQ |
| 7.00 | BLQ |
| 24.0 | BLQ |

BLQ: Below Lower Limit of Quantification (0.5 ng/mL)

### Example 2-48. 5-MeO-DMT-3,3-dimethylsuccinate hydrochloride

| | |
|---|---|
| Dosed Test Article: | 5-MeO—DMT- |
| | 3,3-dimethylsuccinate hydrochloride |
| Dose Route: | Oral |
| Nominal Dose Concentration: | 10 mg/Kg |
| Analyte: | 5-MeO—DMT |

Chemical name: 4-(3-(2-(dimethylamino)ethyl)-5-methoxy-1H-indol-1-yl)-2,2-dimethyl-4-oxobutanoic acid HCl salt
Structural class: amide prodrug
Mechanistic class: presumed pH-dependent cyclization

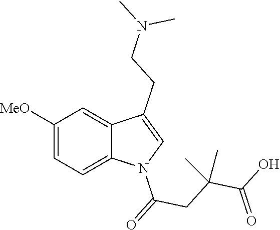 [SMILES: COc1ccc2c(c1)c(CCN(C)C)cn2C(=O)CC(C)(C)C(=O)O]

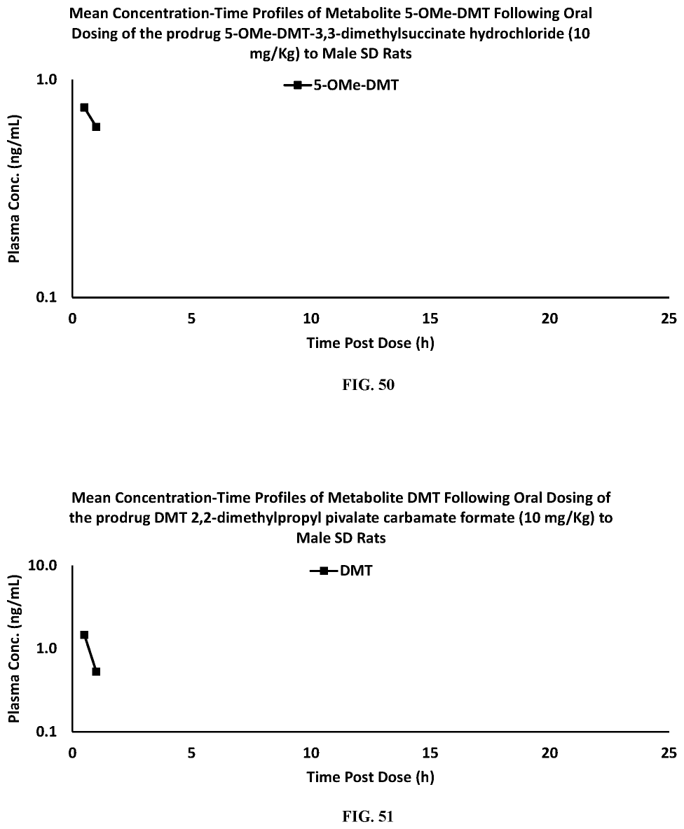

FIG. 50. Mean Concentration-Time Profiles of Metabolite 5-MeO-DMT Following Oral Dosing of the prodrug 5-MeO-DMT-3,3-dimethylsuccinate hydrochloride (10 mg/Kg) to Male SD Rats

TABLE 2-48
5-MeO—DMT PK Parameters
Mean* Pharmacokinetic Parameters

| PK Parameter | 5-MeO—DMT |
|---|---|
| Cmax (ng/mL) | 0.692 |
| Tmax (h) | 1.00 |
| MRT (h) | 0.765 |
| Tlast (h) | 1.00 |
| AUC0-last (h * ng/mL) | 0.449 |
| AUC0-24 (h * ng/mL) | NC |
| AUC0-inf (h * ng/mL) | NC |
| T½ (h) | NC |

*Median calculated for Tmax and Tlast.
NC: Not Calculated

### Example 2-49. DMT 2,2-dimethylpropyl pivalate carbamate formate

| | |
|---|---|
| Dosed Test Article: | DMT 2,2-dimethylpropyl |
| | pivalate carbamate formate |
| Dose Route: | Oral |
| Nominal Dose Concentration: | 10 mg/Kg |
| Analyte: | DMT |

Chemical name: 2,2-Dimethyl-3-(pivaloyloxy)propyl 3-(2-(dimethylamino)ethyl)-1H-indole-1-carboxylate formate
Structural class: carbamate prodrug
Mechanistic class: presumed carboxyesterase+intramolecular cyclization

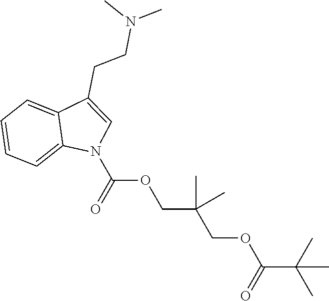 [SMILES: CN(C)CCc1cn(C(=O)OCC(C)(C)COC(=O)C(C)(C)C)c2ccccc12]

FIG. 51. Mean Concentration-Time Profiles of Metabolite DMT Following Oral Dosing of the prodrug DMT 2,2-dimethylpropyl pivalate carbamate formate (10 mg/Kg) to Male SD Rats

TABLE 2-49
DMT PK Parameters
Mean* Pharmacokinetic Parameters

| PK Parameter | DMT |
|---|---|
| Cmax (ng/mL) | 1.47 |
| Tmax (h) | 0.50 |
| MRT (h) | 0.528 |
| Tlast (h) | 0.50 |
| AUC0-last (h * ng/mL) | 0.522 |
| AUC0-24 (h * ng/mL) | NC |
| AUC0-inf (h * ng/mL) | NC |
| T½ (h) | NC |

*Median calculated for Tmax and Tlast.
NC: Not Calculated

### Example 2-50. DMT methyl alcohol

| | |
|---|---|
| Dosed Test Article: | DMT methyl alcohol |
| Dose Route: | Oral |
| Nominal Dose Concentration: | 10 mg/Kg |
| Analyte: | DMT |

Chemical name: (3-(2-(dimethylamino)ethyl)-1H-indol-1-yl)methanol
Structural class: methyleneoxy prodrug
Mechanistic class: presumed chemical breakdown

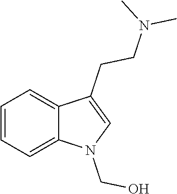 [SMILES: CN(C)CCc1cn(CO)c2ccccc12]

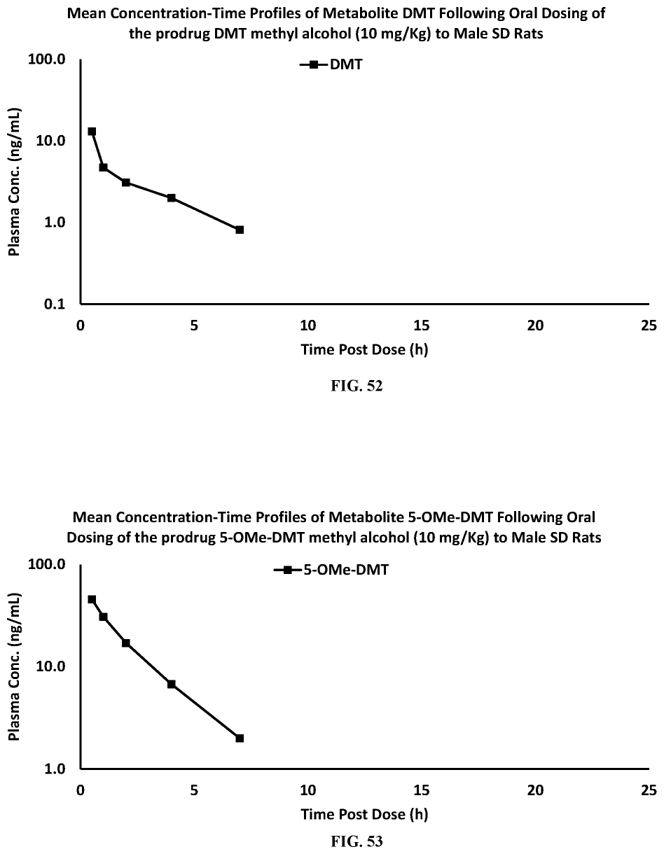

FIG. 52. Mean Concentration-Time Profiles of Metabolite DMT Following Oral Dosing of the prodrug DMT methyl alcohol (10 mg/Kg) to Male SD Rats

TABLE 2-50
DMT PK Parameters
Mean* Pharmacokinetic Parameters

| PK Parameter | DMT |
|---|---|
| Cmax (ng/mL) | 13.1 |
| Tmax (h) | 0.50 |
| MRT (h) | 1.21 |
| Tlast (h) | 2.00 |
| AUC0-last (h * ng/mL) | 14.8 |
| AUC0-24 (h * ng/mL) | NC |
| AUC0-inf (h * ng/mL) | 28.4 |
| T½ (h) | 2.52 |

*Median calculated for Tmax and Tlast.
NC: Not Calculated

### Example 2-51. 5-MeO-DMT methyl alcohol

| | |
|---|---|
| Dosed Test Article: | 5-MeO—DMT methyl alcohol |
| Dose Route: | Oral |
| Nominal Dose Concentration: | 10 mg/Kg |
| Analyte: | 5-MeO—DMT |

Chemical name: (3-(2-(Dimethylamino)ethyl)-5-methoxy-1H-indol-1-yl)methanol
Structural class: methyleneoxy prodrug
Mechanistic class: presumed chemical breakdown

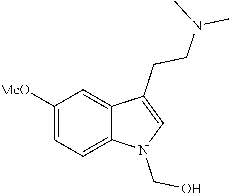 [SMILES: COc1ccc2c(c1)c(CCN(C)C)cn2CO]

FIG. 53. Mean Concentration-Time Profiles of Metabolite 5-MeO-DMT Following Oral Dosing of the prodrug 5-MeO-DMT methyl alcohol (10 mg/Kg) to Male SD Rats

TABLE 2-51
5-MeO—DMT PK Parameters
Mean* Pharmacokinetic Parameters

| PK Parameter | 5-MeO—DMT |
|---|---|
| Cmax (ng/mL) | 46.0 |
| Tmax (h) | 0.50 |
| MRT (h) | 1.89 |
| Tlast (h) | 7.00 |
| AUC0-last (h * ng/mL) | 85.8 |
| AUC0-24 (h * ng/mL) | NC |
| AUC0-inf (h * ng/mL) | 96.0 |
| T½ (h) | 1.70 |

*Median calculated for Tmax and Tlast.
NC: Not Calculated

### Example 2-52. 5-MeO-DMT carboxy-isopropyl valinate di-trifluoroacetate

| | |
|---|---|
| Dosed Test Article: | 5-MeO—DMT carboxy-isopropyl |
| | valinate di-trifluoroacetate |
| Dose Route: | Oral |
| Nominal Dose Concentration: | 10 mg/Kg |
| Analyte: | 5-MeO—DMT |

Chemical name: 1-(((S)-2-amino-3-methylbutanoyl)oxy)-2-methylpropyl 3-(2-(dimethylamino)ethyl)-5-methoxy-1H-indole-1-carboxylate di-trifluoroacetate
Structural class: acyloxymethyl carbamate prodrug
Mechanistic class: presumed carboxyesterase+chemical breakdown

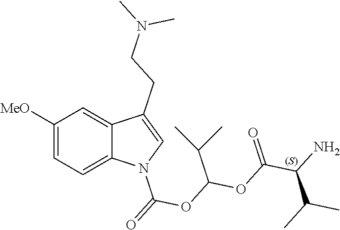

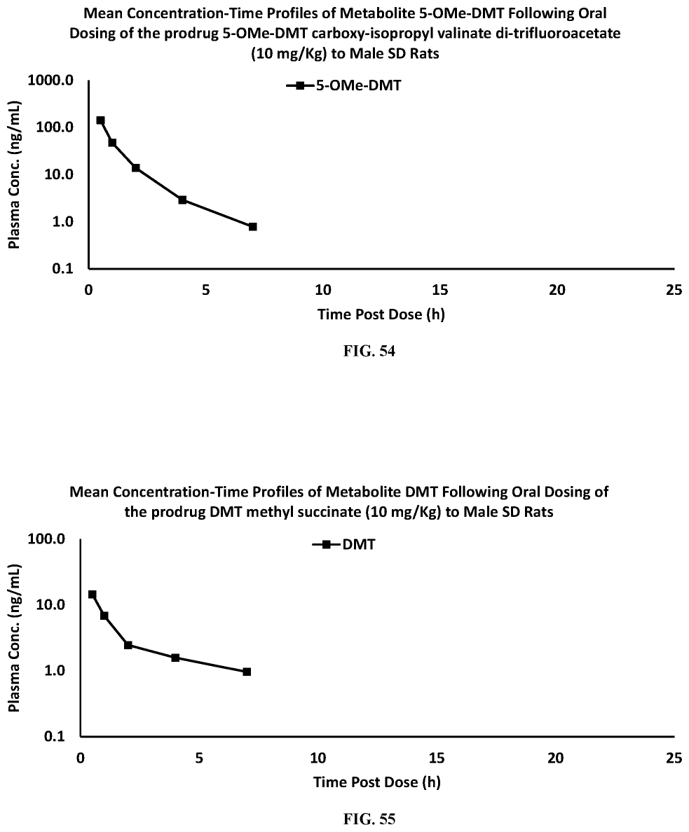

FIG. 54. Mean Concentration-Time Profiles of Metabolite 5-MeO-DMT Following Oral Dosing of the prodrug 5-MeO-DMT carboxy-isopropyl valinate di-trifluoroacetate (10 mg/Kg) to Male SD Rats

TABLE 2-52
5-MeO—DMT PK Parameters
Mean* Pharmacokinetic Parameters

| PK Parameter | 5-MeO—DMT |
|---|---|
| Cmax (ng/mL) | 143 |
| Tmax (h) | 0.50 |
| MRT (h) | 1.00 |
| Tlast (h) | 4.00 |
| AUC0-last (h * ng/mL) | 133 |
| AUC0-24 (h * ng/mL) | NC |
| AUC0-inf (h * ng/mL) | 135 |
| T½ (h) | 0.904 |

*Median calculated for Tmax and Tlast.
NC: Not Calculated

### Example 2-53. DMT methyl succinate

| | |
|---|---|
| Dosed Test Article: | DMT methyl succinate |
| Dose Route: | Oral |
| Nominal Dose Concentration: | 10 mg/Kg |
| Analyte: | DMT |

Chemical name: methyl 4-(3-(2-(dimethylamino)ethyl)-1H-indol-1-yl)-4-oxobutanoate
Structural class: amide prodrug
Mechanistic class: presumed carboxyesterase+pH-dependent cyclization

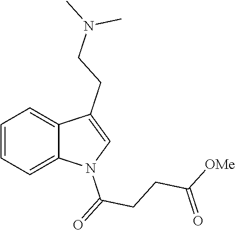 [SMILES: COC(=O)CCC(=O)n1cc(CCN(C)C)c2ccccc21]

FIG. 55. Mean Concentration-Time Profiles of Metabolite DMT Following Oral Dosing of the prodrug DMT methyl succinate (10 mg/Kg) to Male SD Rats

TABLE 2-53
DMT PK Parameters
Mean* Pharmacokinetic Parameters

| PK Parameter | DMT |
|---|---|
| Cmax (ng/mL) | 14.6 |
| Tmax (h) | 0.50 |
| MRT (h) | 1.75 |
| Tlast (h) | 7.00 |
| AUC0-last (h * ng/mL) | 20.7 |
| AUC0-24 (h * ng/mL) | NC |
| AUC0-inf (h * ng/mL) | 24.5 |
| T½ (h) | 2.60 |

*Median calculated for Tmax and Tlast.

### Example 2-54. 5-MeO-DMT methyl succinate

| | |
|---|---|
| Dosed Test Article: | 5-MeO—DMT methyl succinate |
| Dose Route: | Oral |
| Nominal Dose Concentration: | 10 mg/Kg |
| Analyte: | 5-MeO—DMT |

Chemical name: methyl 4-(3-(2-(dimethylamino)ethyl)-5-methoxy-1H-indol-1-yl)-4-oxobutanoate
Structural class: amide prodrug
Mechanistic class: presumed carboxyesterase+pH-dependent cyclization

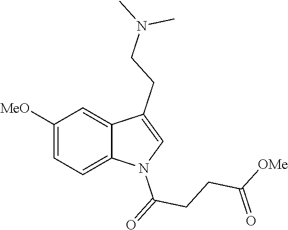 [SMILES: COC(=O)CCC(=O)n1cc(CCN(C)C)c2cc(OC)ccc21]

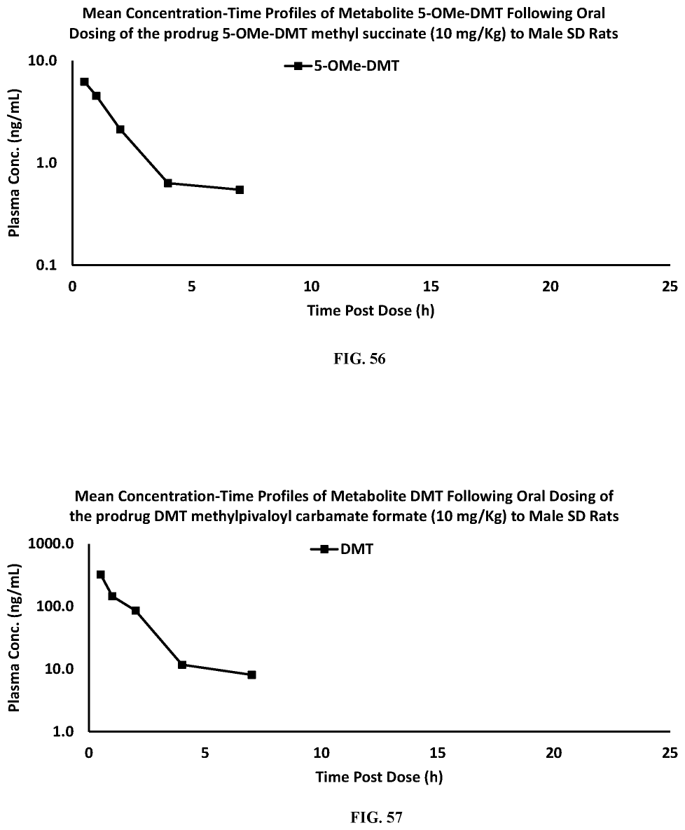

FIG. 56. Mean Concentration-Time Profiles of Metabolite 5-MeO-DMT Following Oral Dosing of the prodrug 5-MeO-DMT methyl succinate (10 mg/Kg) to Male SD Rats

TABLE 2-54
5-MeO—DMT PK Parameters
Mean* Pharmacokinetic Parameters

| PK Parameter | 5-MeO—DMT |
|---|---|
| Cmax (ng/mL) | 6.23 |
| Tmax (h) | 0.50 |
| MRT (h) | 1.37 |
| Tlast (h) | 4.00 |
| AUC0-last (h * ng/mL) | 10.4 |
| AUC0-24 (h * ng/mL) | NC |
| AUC0-inf (h * ng/mL) | 15.1 |
| T½ (h) | 1.53 |

*Median calculated for Tmax and Tlast.

### Example 2-55. DMT methylpivaloyl carbamate formate

| | |
|---|---|
| Dosed Test Article: | DMT methylpivaloyl carbamate formate |
| Dose Route: | Oral |
| Nominal Dose Concentration: | 10 mg/Kg |
| Analyte: | DMT |

Chemical name: (pivaloyloxy)methyl 3-(2-(dimethylamino)ethyl)-1H-indole-1-carboxylate diformate
Structural class: carbamate prodrug
Mechanistic class: presumed carboxyesterase+chemical breakdown

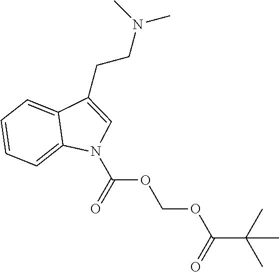 [SMILES: CN(C)CCc1cn(C(=O)OCOC(=O)C(C)(C)C)c2ccccc12]

FIG. 57. Mean Concentration-Time Profiles of Metabolite DMT Following Oral Dosing of the prodrug DMT methylpivaloyl carbamate formate (10 mg/Kg) to Male SD Rats

TABLE 2-55
DMT PK Parameters
Mean* Pharmacokinetic Parameters

| PK Parameter | DMT |
|---|---|
| Cmax (ng/mL) | 324 |
| Tmax (h) | 0.50 |
| MRT (h) | 1.48 |
| Tlast (h) | 7.00 |
| AUC0-last (h * ng/mL) | 441 |
| AUC0-24 (h * ng/mL) | NC |
| AUC0-inf (h * ng/mL) | 462 |
| T½ (h) | 1.64 |

*Median calculated for Tmax and Tlast.

### Example 2-56. Glutarate Prodrug of DMT

| | |
|---|---|
| Dosed Test Article: | Glutarate prodrug of DMT |
| Dose Route: | Oral |
| Nominal Dose Concentration: | 10 mg/Kg |
| Analyte: | DMT |

Chemical name: 5-(3-(2-(dimethylamino)ethyl)-1H-indol-1-yl)-5-oxopentanoic acid
Structural class: amide prodrug
Mechanistic class: presumed pH-dependent cyclization

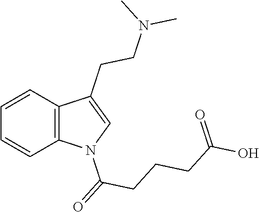 [SMILES: CN(C)CCc1cn(C(=O)CCCC(=O)O)c2ccccc12]

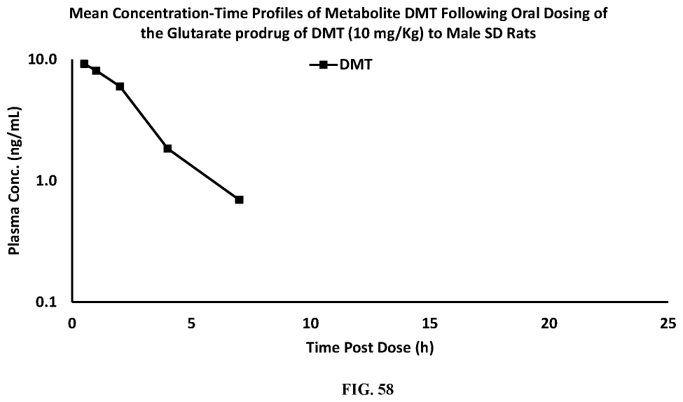

FIG. 58. Mean Concentration-Time Profiles of Metabolite DMT Following Oral Dosing of the Glutarate prodrug of DMT (10 mg/Kg) to Male SD Rats

TABLE 2-56
DMT PK Parameters
Mean* Pharmacokinetic Parameters

| PK Parameter | DMT |
|---|---|
| Cmax (ng/mL) | 9.19 |
| Tmax (h) | 0.50 |
| MRT (h) | 1.92 |
| Tlast (h) | 7.00 |
| AUC0-last (h * ng/mL) | 24.2 |
| AUC0-24 (h * ng/mL) | NC |
| AUC0-inf (h * ng/mL) | 26.2 |
| T½ (h) | 1.57 |

*Median calculated for Tmax and Tlast.

A comparison of the results from Example 2-3 through Example 2-37 reveals that various derivative forms of DMT or 5-OMe-DMT described herein have vastly different pharmacokinetic properties. Oral administration of the compounds tested in Examples 2-3 through Example 2-37 resulted in total measured bodily plasma exposure to DMT or 5-OMe-DMT spanning a range of several orders of magnitude when comparing different DMT or 5-OMe-DMT derivative compounds. These results were unexpected and not predictable based solely on structural knowledge of the compounds.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A compound of Formula (Ia), or a pharmaceutically acceptable salt thereof: 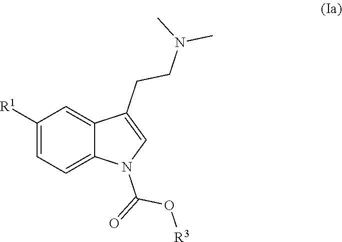
wherein:
R¹ is methoxy or hydrogen;
R³ is alkyl or cycloalkyl, each of which is unsubstituted or substituted with —N(R¹⁸)R¹⁹, heterocycloalkyl, or heteroaryl, wherein heterocycloalkyl and heteroaryl are unsubstituted or substituted with one or more alkyl; and
each of R¹⁸ and R¹⁹ is independently hydrogen or alkyl;
or R¹⁸ and R¹⁹ together with the atom to which they are attached form a heterocyclylalkyl ring that is substituted or unsubstituted with one or more alkyl.

2. The compound of claim 1, or a pharmaceutically acceptable salt thereof, wherein:
R³ is alkyl, which is unsubstituted or substituted with —N(R¹⁸)R¹⁹, heterocycloalkyl, or heteroaryl, wherein heterocycloalkyl and heteroaryl are unsubstituted or substituted with one or more alkyl.

3. The compound of claim 1, or a pharmaceutically acceptable salt thereof, wherein:
R³ is alkyl, which is unsubstituted or substituted with —N(R¹⁸)R¹⁹.

4. The compound of claim 1, or a pharmaceutically acceptable salt thereof, wherein:
R³ is methyl, ethyl, n-propyl, isopropyl, n-butyl, tert-butyl, isobutyl, n-pentyl, or 3-methyl-1-butyl.

5. The compound of claim 1, or a pharmaceutically acceptable salt thereof, wherein:
R³ is ethyl, n-propyl, isopropyl, n-butyl, tert-butyl, or isobutyl.

6. The compound of claim 1, or a pharmaceutically acceptable salt thereof, wherein:
R³ is alkyl, which is substituted with heteroaryl, which is unsubstituted or substituted with one or more alkyl.

7. The compound of claim 1, or a pharmaceutically acceptable salt thereof, wherein:
R³ is

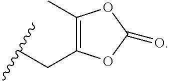 [SMILES: *Cc1oc(=O)oc1C]

8. The compound of claim 1, or a pharmaceutically acceptable salt thereof, wherein:
R¹ is hydrogen, and R³ is methyl, ethyl, n-propyl, isopropyl, n-butyl, tert-butyl, isobutyl, n-pentyl, or 3-methyl-1-butyl;
or R¹ is methoxy, and R³ is methyl, ethyl, n-propyl, isopropyl, n-butyl, tert-butyl, isobutyl, n-pentyl, or 3-methyl-1-butyl.

9. The compound of claim 1, or a pharmaceutically acceptable salt thereof, wherein:
R¹ is hydrogen, and R³ is ethyl;
or R¹ is methoxy, and R³ is ethyl.

10. The compound of claim 1, or a pharmaceutically acceptable salt thereof, wherein the compound has the following structure:

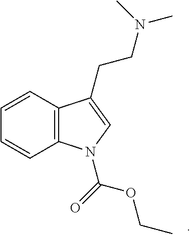 [SMILES: CCOC(=O)n1cc(CCN(C)C)c2ccccc21]

11. The compound of claim 1, or a pharmaceutically acceptable salt thereof, wherein the compound has the following structure:

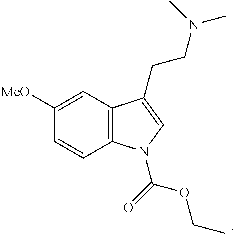 [SMILES: CCOC(=O)n1cc(CCN(C)C)c2cc(OC)ccc21]

12. The compound of claim 1, or a pharmaceutically acceptable salt thereof, wherein the compound has the following structure:

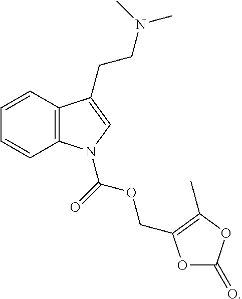 [SMILES: Cc1oc(=O)oc1COC(=O)n1cc(CCN(C)C)c2ccccc21]

13. The compound of claim 1, or a pharmaceutically acceptable salt thereof, wherein the compound has the following structure:

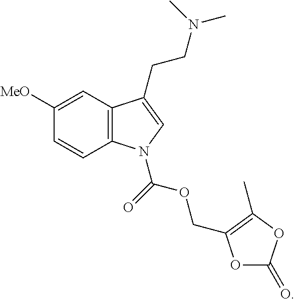 [SMILES: COc1ccc2c(c1)c(CCN(C)C)cn2C(=O)OCc1oc(=O)oc1C]

14. The compound of claim 1, or a pharmaceutically acceptable salt thereof, wherein the compound has one of the following structures:

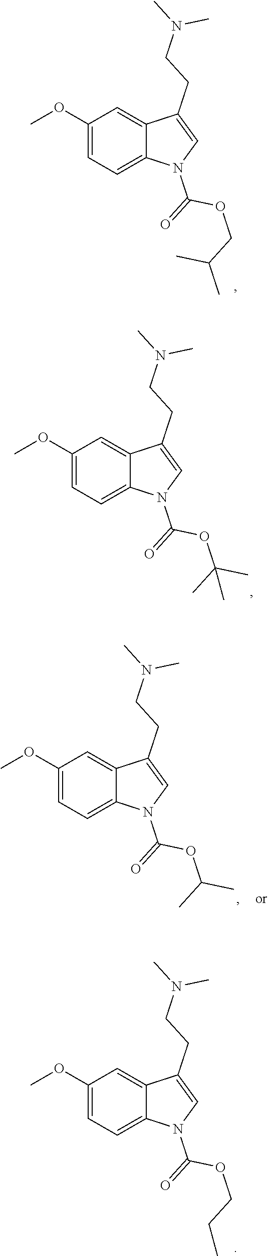

15. The compound of claim 1, or a pharmaceutically acceptable salt thereof, wherein the compound has one of the following structures:

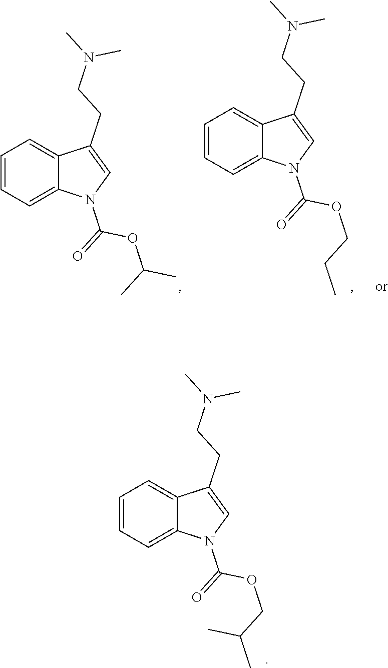 [SMILES: CC(C)OC(=O)n1cc(CCN(C)C)c2ccccc21 ; CCCOC(=O)n1cc(CCN(C)C)c2ccccc21 ; CC(C)COC(=O)n1cc(CCN(C)C)c2ccccc21]

16. The compound of claim 1, or a pharmaceutically acceptable salt thereof, wherein:
R³ is cycloalkyl, which is unsubstituted or substituted with —N(R¹⁸)R¹⁹, heterocycloalkyl, or heteroaryl, which is unsubstituted or substituted with one or more alkyl.

17. The compound of claim 16, wherein the compound has the structure of Formula (Ic), or a pharmaceutically acceptable salt thereof:

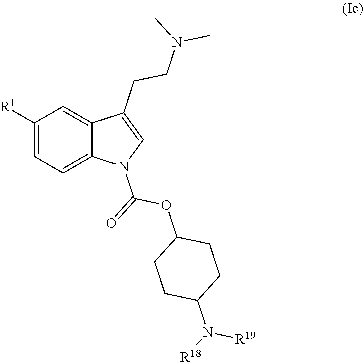

18. The compound of claim 17, or a pharmaceutically acceptable salt thereof, wherein:
each of R¹⁸ and R¹⁹ is independently hydrogen, methyl, ethyl, n-propyl, isopropyl, cyclopropyl, or tert-butyl;
or R¹⁸ and R¹⁹ together with the atom to which they are attached form a azetidine ring, a pyrrolidine ring, or a piperidine ring.

19. The compound of claim 17, or a pharmaceutically acceptable salt thereof, wherein:
each of R¹⁸ and R¹⁹ is hydrogen;
or each of R¹⁸ and R¹⁹ is alkyl;
or R¹⁸ is hydrogen, and R¹⁹ is alkyl;
or R¹⁸ and R¹⁹ together with the atom to which they are attached form an azetidine ring, a pyrrolidine ring, or a piperidine ring.

20. The compound of claim 17, or a pharmaceutically acceptable salt thereof, wherein the compound has one of the following structures:

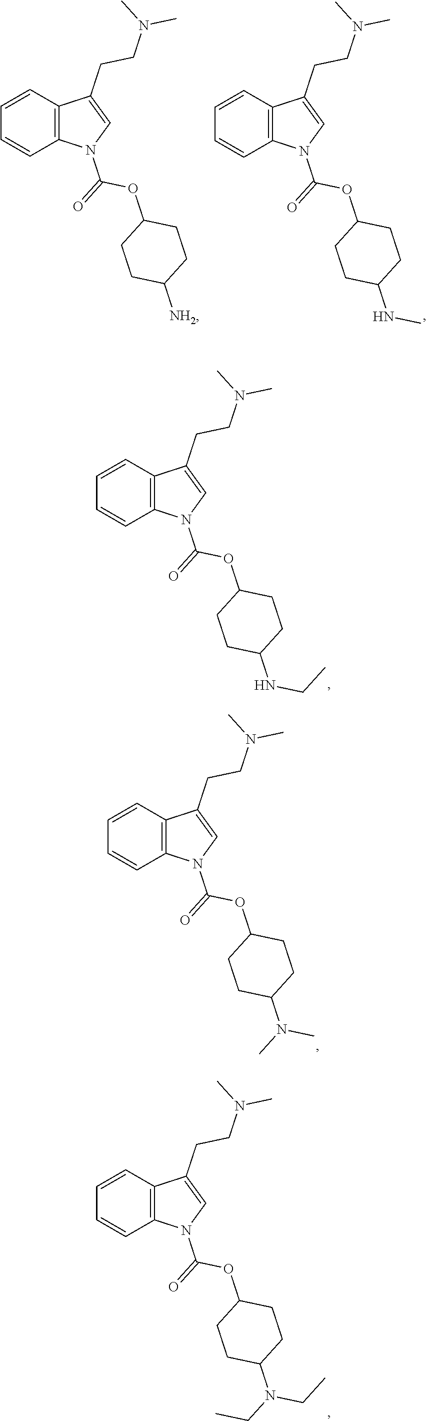

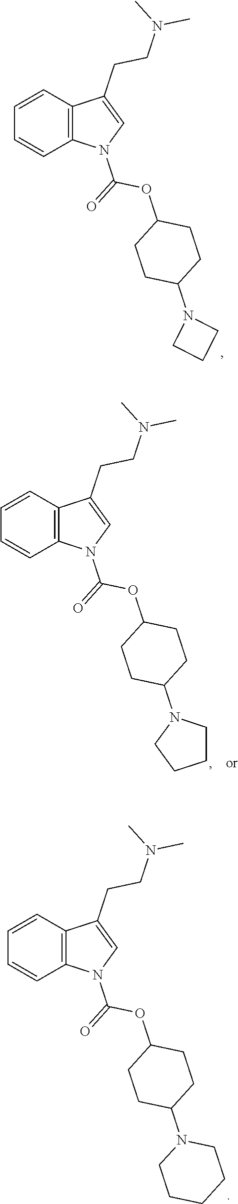 [SMILES: CN(C)CCc1cn(C(=O)OC2CCC(N3CCC3)CC2)c2ccccc12 ; CN(C)CCc1cn(C(=O)OC2CCC(N3CCCC3)CC2)c2ccccc12 ; CN(C)CCc1cn(C(=O)OC2CCC(N3CCCCC3)CC2)c2ccccc12]

21. The compound of claim 17, or a pharmaceutically acceptable salt thereof, wherein the compound has one of the following structures:

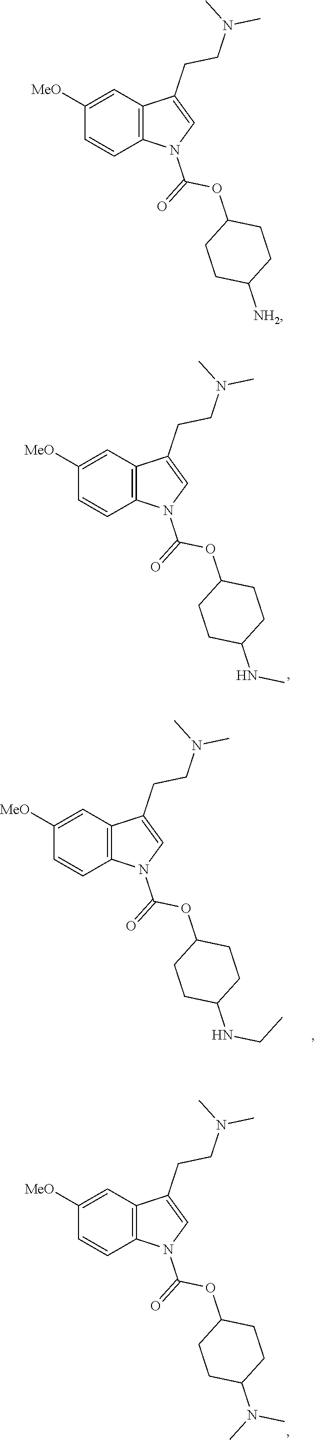 [SMILES: COc1ccc2c(c1)c(CCN(C)C)cn2C(=O)OC1CCC(N)CC1 ; CNC1CCC(OC(=O)n2cc(CCN(C)C)c3cc(OC)ccc32)CC1 ; CCNC1CCC(OC(=O)n2cc(CCN(C)C)c3cc(OC)ccc32)CC1 ; COc1ccc2c(c1)c(CCN(C)C)cn2C(=O)OC1CCC(N(C)C)CC1]

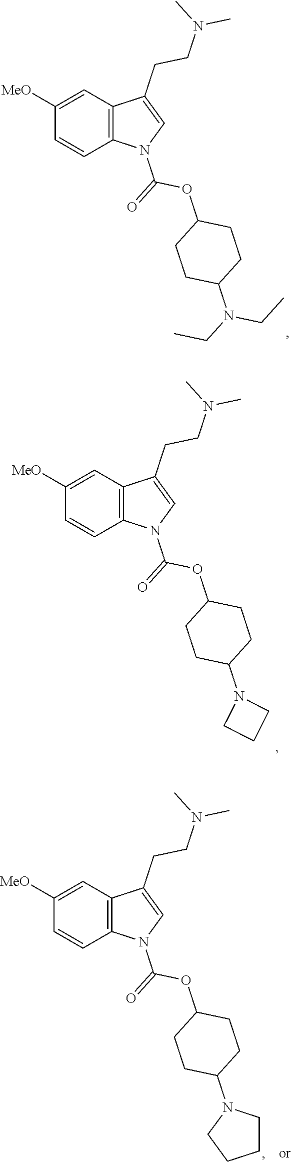

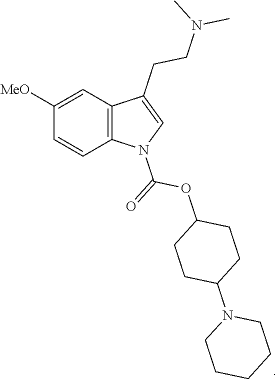 [SMILES: COc1ccc2c(c1)c(CCN(C)C)cn2C(=O)OC1CCC(N2CCCCC2)CC1]

22. A pharmaceutically composition comprising a compound according to claim 1, or a pharmaceutically acceptable salt thereof, and a pharmaceutically acceptable excipient.

23. A method of treating major depression in a human comprising administering an effective amount of a compound of claim 1, or a pharmaceutically acceptable salt thereof, to the human in need thereof, wherein the administration of the effective amount of the compound of claim 1 provides blood plasma concentrations of N,N-dimethyltryptamine (DMT) or 5-methoxy-N,N-dimethyltryptamine (5-MeO-DMT) in the human that are effective for the treatment of major depression.
